(19) United States
(12) Patent Application Publication
(10) Pub. No.: US 2011/0105389 A1
(43) Pub. Date: May 5, 2011

(54) Macrocyclic Ghrelin Receptor Antagonists and Inverse Agonists and Methods of Using the Same

(76) Inventors: Hamid R. Hoveyda, Bruxelles (BE); Eric Marsault, Quebec (CA); Helmut Thomas, Quebec (CA); Graeme Fraser, Rixensart (BE); Sylvie Beaubien, Quebec (CA); Axel Mathieu, Quebec (CA); Julien Beignet, Quebec (CA); Marc-André Bonin, Quebec (CA); Serge Phoenix, Quebec (CA); David Drutz, Chapel Hill, NC (US); Mark Peterson, Quebec (CA); Sophie Beauchemin, Quebec (CA); Martin Brassard, Quebec (CA); Martin Vezina, Quebec (CA)

(21) Appl. No.: 12/916,205

(22) Filed: Oct. 29, 2010

Related U.S. Application Data
(60) Provisional application No. 61/256,727, filed on Oct. 30, 2009.

(51) Int. Cl. A61K 38/12; C07K 5/08; C07K 5/087; A61K 38/26; A61P 3/04; A61P 3/10; A61P 25/00; A61P 25/32; A61P 9/00; A61P 1/00; A61P 3/00; A61P 1/16; A61P 5/00; A61P 25/30

(52) U.S. Cl. 514/4.8; 530/331; 514/21.1; 514/11.7; 514/6.9; 514/17.5; 514/17.7

(57) ABSTRACT

The present invention provides novel conformationally-defined macrocyclic compounds that have been demonstrated to be selective modulators of the ghrelin receptor (GRLN, growth hormone secretagogue receptor, GHS-R1a and subtypes, isoforms and/or variants thereof). Methods of synthesizing the novel compounds are also described herein. These compounds are useful as antagonists or inverse agonists of the ghrelin receptor and as medicaments for treatment and prevention of a range of medical conditions including, but not limited to, metabolic and/or endocrine disorders, obesity and obesity-associated disorders, appetite or eating disorders, addictive disorders, cardiovascular disorders, gastrointestinal disorders, genetic disorders, hyperproliferative disorders, central nervous system disorders and inflammatory disorders.

### CROSS REFERENCE TO RELATED APPLICATION

This application claims priority to U.S. Provisional Application Ser. No. 61/256,727, filed Oct. 30, 2009, the disclosure of which is incorporated herein by reference in its entirety.

### FIELD OF THE INVENTION

The present invention relates to novel conformationally-defined macrocyclic compounds that have been demonstrated to function as antagonists or inverse agonists of the ghrelin (growth hormone secretagogue) receptor (GRLN, GHS-R1a). The invention also relates to intermediates of these compounds, pharmaceutical compositions containing these compounds and methods of using the compounds. These novel macrocyclic compounds are useful as therapeutics for a range of indications including metabolic and/or endocrine disorders, obesity and obesity-associated disorders, appetite or eating disorders, addictive disorders cardiovascular disorders, gastrointestinal disorders, genetic disorders, hyperproliferative disorders, central nervous system disorders and inflammatory disorders.

### BACKGROUND OF THE INVENTION

The improved understanding of various physiological regulatory pathways enabled through the research efforts in genomics and proteomics has begun to impact the discovery of novel pharmaceutical agents. In particular, the identification of key receptors and their endogenous ligands has created new opportunities for exploitation of these receptor/ligand pairs as therapeutic targets. For example, ghrelin is a recently characterized 28-amino acid peptide hormone that has been shown to mediate a variety of important physiological functions. (Kojima, M.; Hosoda, H.; et al. Nature 1999, 402, 656-660.) A novel characteristic of the structure is the presence of an n-octanoyl group on Ser³ that appears to be relevant to ghrelin's activity. This peptide has been demonstrated to be the endogenous ligand for a previously orphan G protein-coupled receptor (GPCR), type 1 growth hormone secretatogue receptor (hGHS-R1a). (Howard, A. D.; Feighner, S. D.; Cully, D. F.; et al. Science 1996, 273, 974-977.) GHS-R1a has recently been reclassified as the ghrelin receptor (GRLN) in recognition of its endogenous ligand (Davenport, A. P.; et al. Pharmacol. Rev. 2005, 57, 541-546).

Even prior to the isolation of this receptor and its endogenous peptide ligand, a significant amount of research was devoted to finding agents that can stimulate growth hormone (GH) secretion. The proper regulation of human GH has importance not only for proper body growth, but also for a range of other critical physiological effects. GH and other GH-stimulating peptides, such as growth hormone-releasing hormone (GHRH) and growth hormone releasing factor (GRF), as well as their derivatives and analogues, are administered via injection. Therefore, to better take advantage of these positive effects, attention was focused on the development of orally active therapeutic agents that would increase GH secretion, termed GH secretagogues (GHS). Additionally, use of these agents was expected to be able to more closely mimic the pulsatile physiological release of GH.

Beginning with the identification of the growth hormone-releasing peptides (GHRP) in the late 1970's (Bowers, C. Y. Curr. Opin. Endocrinol. Diabetes 2000, 7, 168-174; Camanni, F.; Ghigo, E.; Arvat, E. Front. Neurosci. 1998, 19, 47-72; Locatelli, V.; Torsello, A. Pharmacol. Res. 1997, 36, 415-423), a host of agents have been studied for their potential to act as GHS. In addition to their stimulation of GH release and concomitant positive effects in that regard, GHS were projected to have utility in a variety of other disorders, including the treatment of wasting conditions (cachexia) as seen in HIV patients and cancer-induced anorexia, musculoskeletal frailty in the elderly, and growth hormone deficient diseases. Many efforts over the past 25 years have yielded a number of potent, orally available GHS. (Cordido, F.; Isidro, M. L.; Nemina, R.; Sangiao-Alvarellos, S. Curr. Drug Disc. Tech. 2009, 6, 34-42; Isidro, M. L.; Cordido, F. Comb. Chem. High Throughput Screen. 2006, 9, 178-180; Smith, R. G.; Sun, Y. X.; Beatancourt, L.; Asnicar, M. Best Pract. Res. Clin. Endocrinol. Metab. 2004, 18, 333-347; Fehrentz, J.-A.; Martinez, J.; Boeglin, D.; Guerlavais, V.; Deghenghi, R. IDrugs 2002, 5, 804-814; Svensson, J. Exp. Opin. Ther. Patents 2000, 10, 1071-1080; Nargund, R. P.; Patchett, A. A.; Bach, M. A.; Murphy, M. G.; Smith, R. G. J. Med. Chem. 1998, 41, 3103-3127; Ghigo, E; Arvat, E.; Camanni, F. Ann. Med. 1998, 30, 159-168.) These include small peptides, such as hexarelin (Zentaris) and ipamorelin (Novo Nordisk), as well as small molecules such as capromorelin (Pfizer), L-252,564 (Merck), MK-0677 (Merck), NN703 (tabimorelin, Novo Nordisk), G-7203 (Genentech), S-37435 (Kaken) and SM-130868 (Sumitomo). However, clinical tests with such agents have rendered disappointing results due to, among other things, lack of efficacy over prolonged treatment or undesired side effects, including irreversible inhibition of cytochrome P450 enzymes. (Zdravkovic M.; Olse, A. K.; Christiansen, T.; et al. Eur. J. Clin. Pharmacal. 2003, 58, 683-688.)

The cloning of the human receptor, which was actually enabled through the use of a synthetic GHS, and the subsequent identification of ghrelin have opened a variety of new chemical areas for investigation on both agonists and antagonists (Camino, P. A. Exp. Opin. Ther. Patents 2002, 12, 1599-1618). In particular, the ghrelin peptide has been found to have multiple other physiological functions apart from the stimulation of GH release, including regulation of food intake and appetite, promotion of weight gain, control of energy balance, and modulation of gastrointestinal (GI) motility, gastric acid secretion and glucose homeostasis. The hormone has also been linked to control of circadian rhythm and memory. Ghrelin appears to also play a role in bone metabolism and inflammatory processes. (Van der Lely, A. J.; Tschöp, M,; Heiman, M. L.; Ghigo, E. Endocrine Rev. 2004, 25, 426-457; Inui, A.; Asakawa, A.; Bowers, C. Y.; Mantovani, G.; Laviano, A.; Meguid, M. M.; Fujimiya, M. FASEB J. 2004, 18, 439-456; Diano, S. Farr, S. A.; Benoit, S. C.; et al. Nat. Neuroscience 2006, 9, 381-388; Kojima, K.; Kangawa, K. Nat. Clin. Pract. Endocrinol. Metab. 2006, 2, 80-88; Kaiya, H.; Miyazato, M.; Kangawa, K.; Peter, R. E.; Unniappan, S. Comp. Biochem. Physiol. A 2008, 149, 109-128.)

Due to these myriad physiological effects, modulation of the ghrelin receptor has come under increasing study for therapeutic indications apart from those related to the GH secretory function (Dodge, J. A.; Heiman, M. L. Ann. Rep. Med. Chem. 2003, 38, 81-88.). For example, Intl. Pat. Appl. WO 2006/009645 and WO 2006/009674 describe the use of macrocyclic compounds as ghrelin modulators for use in the treatment of gastrointestinal (GI) disorders. Similarly, WO 2006/020930 and WO 2006/023608 describe structurally distinct ghrelin agonists (growth hormone secretagogues) for use in such GI disorders. In addition, Intl. Pat. Appl. WO 2004/09124 and WO 2005/68639 describe modified virus particles derived from short peptide sequences from the N-terminus of ghrelin that can be used as vaccines for treatment of obesity. Another vaccine approach for obesity is described in WO 2004/024183.

Not surprisingly due to the role of ghrelin in the control of appetite and feeding, particular interest has also been sparked in the development of ghrelin antagonists and inverse agonists as new anti-obesity pharmaceutical agents, as indeed has modulation of a number of peptide hormones and their receptors. (Crowley, V. E. F.; Yeo, G. S. H.; O-Rahilly, S. Nat. Rev. Drug Disc. 2002, 1, 276-286; Spanswick, D.; Lee, K. Exp. Opin. Emerging Drugs 2003, 8, 217-237; Horvath, T. L.; Castañeda, T.; Tang-Christensen, M.; Pagotto, U.; Tschöp, M. H. Curr. Pharm. Design 2003, 9, 1383-1395; Higgins, S. C.; Gueorguiev, M.; Korhonits, M. Ann. Med. 2007, 39, 116-136; Carpino, P. A.; Ho, G. Exp. Opin. Ther. Pat. 2008, 18, 1253-1263; Soares, J.-B.; Roncon-Albuquerque, R., Jr.; Leite-Moreira, A. Exp. Opin. Ther. Targets 2008, 12, 1177-1189; Ukkola, O. Curr. Prot. Pept. Sci. 2009, 10, 2-7; Constantino, L.; Barlocco, D. Fut. Med. Chem., 2009, 1, 157-177; Chollet, C.; Meyer, K.; Beck-Sickinger, A. G. J. Pept. Sci. 2009, 15, 711-730.) In contrast to ghrelin agonists, with the precedence in the search for GHS, the field of research on ghrelin antagonists and inverse agonists is significantly less mature. U.S. Patent Application Publ. 2003/0211967 and WO 01/87335 address the use of ghrelin antagonists as treatments for a variety of disease states including obesity and related disorders. Similarly, WO 01/56592 and US 2001/020012 describe the use of ghrelin antagonists for the regulation of food intake. Likewise, WO 2004/004772 describes the use of GHS-R antagonists as a treatment for diabetes, obesity and appetite control. Their use for treatment of intestinal inflammation has also been described (Intl. Pat. Appl. Publ. WO 2004/084943; U.S. Pat. Appl. Publ. 2007/0025991). However, no specific examples of compounds, apart from ghrelin peptide and its analogues, for this purpose are presented in these applications. More recently, oxadiazole ghrelin antagonists have been reported which are also claimed to be effective in improving cognition, memory and other CNS disorders (WO 2005/112903). Modulation of thermoregulation, sleep, appetite, food intake, obesity and other ghrelin-mediated conditions through reduction of ghrelin expression is described in U.S. Pat. Appl. Publ. 2010/0196396.

Ghrelin antagonists and inverse agonists have also been considered for playing a role in the reduction of the incidence of the following obesity-associated conditions including diabetes, complications due to diabetes such as retinopathy, cardiovascular diseases, hypertension, dyslipidemia, osteoarthritis and certain forms of cancer. Indeed, in addition to the anti-obesity effects seen in animal studies, transgenic rats engineered without the GRLN (GHS-R1a) receptor have exhibited reduced food intake, diminished fat deposition, and decreased weight. However, the hormone's involvement in a number of physiological processes, including regulation of cardiovascular function and stress responses as well as growth hormone release, may indicate potential drawbacks to this strategy. Hence, complete lack of ghrelin may not be desirable, but suppression may be sufficient to control obesity and other metabolic disorders. It should be noted that recent studies with ghrelin knockout mice reveal that these animals do not exhibit the expected modifications in size and food intake among other physiological characteristics. (Sun, Y.; Ahmed, S.; Smith, R. G. Mol. Cell Biol. 2003, 23, 7973-7981; Wortley, K. E.; Anderson, K. D.; Garcia, K.; et al. Proc. Natl. Acad. Sci. USA 2004, 101, 8227-8232.)

Ghrelin plays a key role in the regulation of insulin release and glycemia and hence modulators of the ghrelin receptor have application to the treatment of diabetes and metabolic syndrome. (Yada, T.; Dezaki, K. Sone, H.; et al. Curr. Diab. Rep. 2008, 4, 18-23; Pulkkinen, L.; Ukkola, O.; Kolehmainen, M.; Uusitupa, M. Int. J. Pept. 2010, doi: 10.1155/2010/248948.) Ghrelin reduces glucose. stimulated insulin secretion, decreases insulin sensitivity, increases resting/fasting blood glucose levels, shifts energy metabolism from fat to glucose, and indirectly antagonizes insulin dependent CNS regulation of food intake and glucose homeostasis. (Sun, Y.; Asnicar, M.; Smith, R. G. Neuroendocrinol. 2007, 86, 215-228; Dezaki, K.; Sone, H.; Yada, T. Pharmacol. Ther. 2008, 118, 239-249; Tong, J.; Prigeon, R. L.; Davis, H. W.; et al. Diabetes 2010, 59, 2145-2151.). Ghrelin antagonists and/or inverse agonists hence would have beneficial effects for the treatment or prevention of diabetes and related conditions, such as metabolic syndrome.

Recently, BIM-28163 has been reported to function as an antagonist at the GRLN (GHS-R1a) receptor and inhibit receptor activation by native ghrelin. However, this same molecule is a full agonist with respect to stimulating weight gain and food intake. This and related peptidic ghrelin analogues effectively separate the GH-modulating activity of ghrelin from the effects of the peptide on weight gain and appetite. (Halem, H. A.; Taylor, J. E.; Dong, J. Z.; et al. Eur. J. Endocrinol. 2004, 151, S71-S75.) Analogously, the macrocyclic ghrelin agonists described in WO 2006/009645 and WO 2006/009674 report the separation of the GI effects from the GH-release effects in animal models.

In addition to the ghrelin receptor itself, another component of the ghrelin biological pathway, the enzyme ghrelin-O-acyltransferase (GOAT), has been suggested as an anti-obesity target. (Romero, A.; Kirchner, H.; Heppner, K.; et al. Eur. J. Endocrinol. 2010, 163, 1-8; Intl. Pat. Appl. Publ. WO 2008/079705; Gutierrez, J. A.; Solenberg, P. J.; Perkins, D. R.; et al. Proc. Natl. Acad. Sci. 2008, 105, 6320-6325.) GOAT is responsible for the post-translational modification that incorporates the n-octanoyl moiety on Ser³ of ghrelin. As mentioned previously, this acylated form is the active species in vivo. Pentapeptide (Yang, J.; Zhao, T. J.; Goldstein, J. L.; et al. Proc. Natl. Acad. Sci. 2008, 105, 10750-10755), small molecule (BK1114, U.S. Pat. Appl. Publ. 2010/0086955) and bisubstrate (Intl. Pat. Appl. Publ. WO 2010/039461) inhibitors of GOAT have been reported, but this approach is still not yet proven in humans.

Prader-Willi syndrome, the most common form of human syndromic obesity, is characterized paradoxically by GH deficiency and high ghrelin levels that are not decreased after feeding. (Cummings, D. E.; Clement, K.; Purnell, J. Q.; et al. Nat. Med. 2002, 8, 643-644.) Antagonists of the ghrelin receptor would have a role in treating this syndrome as well.

Non-alcoholic fatty liver disease (NAFLD) is a spectrum of pathological conditions characterized by the formation of significant lipid deposits in liver hepatocytes. NAFLD is the most common liver problem in industrialized Western countries, affecting 20-40% of the general population. In patients with type II diabetes, prevalence of NAFLD may be as high as 70% and in obese individuals NAFLD prevalence is 58-74%. NAFLD can progress to non-alcoholic steatohepatitis (NASH), which increases the potential for development of liver cirrhosis. (Angulo, P. New Engl. J. Med. 2002, 346, 1221-1231; Perlernuter, G.; Bigorgne, A.; Cassard-Doulcier, A.-M.; Naveau, S, Nat. Clin. Pract. Endocrinol. Metab. 2007, 3, 458-469; Younossi, Z. M. Aliment. Pharmacol. Ther. 2008, 28, 2-12; Ali, R.; Cusi, K. Ann. Med. 2009, 41, 265-278; Malaguarnera, M.; Di Rosa, M.; Nicoletti, F.; Malaguarnera, L. J. Mol. Med. 2009, 87, 679-695.)

NAFLD can occur with or without inflammation of the liver or liver cell injury or damage, and without a history of excessive alcohol ingestion. It has been suggested that NAFLD represents the hepatic manifestation of metabolic syndrome, but may also predict the development of metabolic syndrome. Although NAFLD has been found in patients without risk factors, individuals with conditions such as diabetes, obesity, hypertension and hypertriglyceridemia are at greatest risk of developing the condition. An inextricable relationship exists between central obesity, steatosis and insulin resistance. Adipokines and ghrelin have been implicated in the pathogenesis of nonalcoholic fatty liver disease through their metabolic and/or anti-inflammatory activity. Emerging data shows a relationship between NAFLD, ghrelin and adipokines. Ghrelin was elevated in patients with NAFLD, primarily those with normal body weight. Peripheral ghrelin induces lipid accumulation in specific abdominal depots, liver and skeletal muscle without affecting superficial subcutaneous white adipose tissue. These effects may be augmented by suppression of spontaneous growth hormone (GH) secretion. In addition, peripheral ghrelin and des-acyl ghrelin induce adipogenesis in hone marrow. Peripheral ghrelin defends accumulated fat in abdominal locations associated with the development of metabolic syndrome (Wells, T. Prog. Lipid Res. 2009, doi:10.1016/j.plipres.2009.04.002). Studies have shown that ghrelin may influence adipocyte metabolism and stimulate adipogenesis. (Depoortere, I. Regul. Pept. 2009, 156, 13-23.). Ghrelin antagonists would therefore be useful in the treatment or prevention of NAFLD and NASH.

Similarly, such agents may have potential for diabetic hyperphagia. Hyperphagia and altered fuel metabolism result from uncontrolled diabetes mellitus in humans. This has been suggested to occur through a combination of elevated ghrelin levels and decreased leptin through the NPY/AGRP pathway. Although levels of ghrelin are essentially the same in healthy and diabetic subjects, the different levels of ghrelin in diabetic hyperphagia could make it difficult to remain on diet therapies and an antagonist could be useful in assisting control. (Ishii, S.; Kamegai, J.; Tamura, H.; Shimizu, T.; Sugihara, H.; Oikawa, S. Endocrinology 2002, 143, 4934-4937; Sindelar, D. K., Mystkowski, P., Marsh, D. J., Palmiter, R. D.; Schwartz, M. W Diabetes 2002, 51, 778-783.)

Ghrelin levels are elevated in cirrhosis and with complications from chronic liver disease, although unlike levels of insulin-like growth factor-1 (IGF-1), they do not correlate to liver function. (Tacke, F.; Brabant, G.; Kruck, E.; Horn, R.; et al. J. Hepatology 2003, 38, 447-454.) Ghrelin antagonists could be useful in controlling these liver diseases. Further, ghrelin and its receptor are overexpressed in numerous cancers. Antagonists would have potential application to treatment of cancer. Intl. Pat. Appl. Publ. WO 02/90387 has described the use of interventionist strategies targeting GHS-R1a as an approach to treatment of cancers of the reproductive system.

For metabolic disorders such as obesity, it has been speculated that due to the critical nature of the food intake process for the survival of the organism, a single agent with a single target may not be sufficient for long term weight control since alternative or redundant pathways can be used to circumvent the affected pathway. Hence, the best therapeutic strategy may be to simultaneously apply multiple agents that target different pathways involved in the feeding/appetite control process (see for example Intl. Pat. Appl. Publ. WO 2006/052608). Indeed, some successful weight-loss therapeutics have been combinations of drugs.

Recently, antagonism of ghrelin has been demonstrated to reduce alcohol consumption. (Kaur, S.; Ryabinin, A. E. Alcohol. Clin. Exp. Res. 2010, 34, 1525-1534.) This is consistent with studies that have shown altered plasma ghrelin levels in alcoholic patients (Wurst, F. M.; Graf, I.; Ehrenthal, H. D.; et al. Alcohol. Clin. Evp. Res. 2007, 31, 2006-2020; Badaoui, A.; De Saeger, C.; Duchemin, J.; Gihousse, D.; de Timary, P.; Starkel, P. Eur. J. Clin. Invest. 2008, 38, 397-403) and reduced alcohol intake in ghrelin knockout mice (Jerlhag, E.; Egecioglu, E.; Landgren, S.; et al. Proc. Natl. Acad. Sci. USA 2009, 106, 11318-11323). Relatedly, reduction of food intake in mice with a disrupted gene or treated with a ghrelin antagonist suggests ghrelin involvement in the incentive and reward system associated with food. (Egecioglu, E.; Jerlhag, E.; Salome, N.; et al. Addict. Biol. 2010, 15, 304-311; Perello, M.; Sakata, I.; Birnbaum, S.; et al. Biol. Psychiatry 2010, 67, 880-886.) Further, dopamine release upon the presence of rewarding food was absent in ghrelin knockout mice. In addition, the ghrelin signaling system appears to be required for a reward from drugs of abuse. (Jerlhag, E.; Egecioglu, E.; Dickson, S. L.; Engel, J. A. Psychopharmacol. 2010, 211, 415-422) Amphetamine- or cocaine-induced stimulation and dopamine release were reduced upon treatment with a ghrelin antagonist. Ghrelin antagonists therefore would have utility for treatment of alcohol-related disorders (Leggio, L. Drug News Perspect, 2010, 23, 157-166.) and other addictive disorders, such as drug dependence (Intl. Pat. Appl. Publ. WO 2009/020419). Despite the potential therapeutic uses for ghrelin antagonists, only a limited number of small molecule ghrelin antagonists have yet been reported in the patent or scientific literature including diaminopyrimidines, tetralin carboxamides, isoxazole carboxamides, β-carbolines, oxadiazoles, pyrazoles, benzofuranylindolones and benzenesulfonamides. (U.S. Pat. Appl. Publ. US 2005/0171131; US 2005/0171132; Intl. Pat. Appl. Publ. WO 2005/030734; WO 2005/112903; WO 2005/48916; WO 2008/008286; WO 2010/092288; WO 2010/092289; Zhao, H.; Xin, Z.; Liu, G.; et al. J. Med. Chem. 2004, 47, 6655-6657; Xin, Z.; Zhao, H.; Serby, M. D.; et al. Bioorg. Med. Chem. Lett. 2005, 15, 1201-1204; Zhao, H.; Xin, Z.; Patel, J. R.; et al. Bioorg. Med. Chem. Lett. 2005, 15, 1825-1828; Liu, B.; Liu, G.; Xin, Z.; et al. Bioorg. Med. Chem. Lett. 2004, 14, 5223-5226; Pasternak, A,; Goble, S. D.; deJesus, R. K.; et al. Bioorg. Med. Chem. Lett. 2009, 19, 6237-6240). WO 2005/114180 describes a number of individual compounds containing heteroaryl core structures, such as isoazoles, 1,2,4-oxadiazoles and 1,2,4-triazoles, as “functional ghrelin antagonists” and their uses as therapeutic agents for the treatment of obesity and diabetes. Other heterocyclic structures, some of which displayed antagonist activity, are reported in WO 2005/035498; WO 2005/097788 and US 2005/0187237.

The remaining known ghrelin antagonists are primarily peptidic in nature (WO 2004/09616, WO 02/08250, WO 03/04518, US 2002/0187938, Pinilla, L.; Barreiro, M. L.; Tena-Sempere, M.; Aguilar E. Neuroendocrinology 2003, 77, 83-90) although antagonists based on nucleic acids have also been disclosed (WO 2004/013274; WO 2005/49828; Helmling, S.; Maasch, C.; Eulberg, D.; et al. Proc. Natl. Acad. Sci. USA 2004, 101, 13174-13179; Shearman, L. P.; Wang, S. P.; Helmling, S.; et al. Endocrinology 2006, 147, 1517-1526). The compounds of the present invention are structurally distinct from all of these previously reported ghrelin antagonist structures. The 14-amino acid compound, vapreotide, a small somatostatin mimetic, was demonstrated to be a ghrelin antagonist. (Deghenghi R, Papotti M, Ghigo E, Muccioli G, Locatelli V. Endocrine 2001, 14, 29-33.) The binding activity of analogues of the cyclic neuropeptide cortistatin to the growth hormone secretatogue receptor has been disclosed (WO 03/004518). These compounds exhibit an IC₅₀ of 24-33 nM. In particular, one of these analogues, EP-01492 (cortistatin 8) has been advanced into preclinical studies for the treatment of obesity as a ghrelin antagonist. (Deghenghi R, Broglio F, Papotti M, et al. Endocrine 2003, 22, 13:18; Sibilia, V.; Muccioli, G.; Deghenghi, R.; et al. J. Neuroendocrinol. 2006, 18, 122-128.)

A limited series of peptides as ghrelin antagonists containing the very specific short octanoylated sequence known to be critical for binding to GHS-R1a has been reported (U.S. Pat. Appl. No. 2002/0187938; Intl. Pat. Appl. No. WO 02/08250). Action of [D-Lys³]-GHRP-6 has been described as a ghrelin antagonist. (Pinilla, L.; Barreiro, M. L.; Tena-Sempere, M.; Aguilar E. Neuroendocrinology 2003, 77, 83-90) More recently, the substance P peptide derivative, L-756,867 (EP-80317, [D-Arg¹,D-Phe⁵,D-Trp^{7,9},Leu¹¹]-substance P), a weak ghrelin antagonist, was demonstrated to be a potent inverse agonist (K_{d/i}=45 nM) to open another potential approach to the treatment of obesity targeting the ghrelin receptor. (Holst, B.; Schwartz, T. W. Trends Pharmacol. Sci. 2004, 25, 113-117; Hoist, B.; Cygankiewicz, A.; Jensen, T. H.; Ankersen, M.; Schwartz, T. W. Mol. Endocrinol. 2003, 17, 2201-2210; Cheng, K.; Wei, I.; Chaung, L.-Y.; et al. J. Endocrinol. 1997, 152, 155-158.) However, the use of this particular agent likely would be limited due to its poor selectivity since it also interacts at the neurokinin-1 and bombesin receptors.

The use of inverse agonists has been suggested to even be of more relevant use for the control of appetite due to the high constitutive activity of the ghrelin receptor. (Hoist, B.; Holliday, N. D.; Bach, A.; Elling, C. E.; Cox, H. M.; Schwartz, T. W. J. Biol. Chem. 2004, 279, 53806-53817.) However, only the L-756,867 peptide and a single pyrrole compound, TM27810, (WO 2004/056869) have been reported to date as inverse agonists.

In fact, it has been argued that it is actually beneficial to have compounds that act as both ghrelin receptor antagonists and inverse agonists in order to best control feeding (Hoist, B. Schwartz, T. J. Clin. Invest. 2006, 116, 637-641). The recent observation that humans possessing a mutation in the ghrelin receptor that impairs constitutive activity are of short stature illustrates the importance of the constitutive activity to the normal in vivo function of this receptor. (Pantel, J.; Legendre, M. Cabrol, S.; et al. J. Clin. Invest. 2006, 116, 760-768.) As shown in the Examples, some compounds of the present invention act as both ghrelin receptor antagonists and inverse agonists.

Although a limited series of macrocyclic peptidomimetics has been previously described as antagonists and inverse agonists of the ghrelin receptor and their uses for the treatment of a variety of disorders summarized (Intl. Pat. Appl. Publ. Nos. WO 2006/046977; 2006/137974), the compounds of the present invention are shown to possess unexpected and more favorable pharmacological properties.

Accordingly, with so few examples of ghrelin antagonists or inverse agonists suitable for pharmacological intervention, there is a need for additional compounds that modulate the ghrelin receptor and suppress ghrelin release.

### SUMMARY OF THE INVENTION

The present invention provides novel conformationally-defined macrocyclic compounds that can function as antagonists or inverse agonists of the ghrelin (growth hormone secretagogue) receptor (GRLN, GHS-R1a).

According to aspects of the present invention, the present invention relates to compounds according to formula (I):

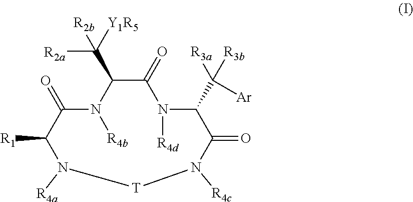

or a pharmaceutically acceptable salt thereof, wherein:

T is selected from

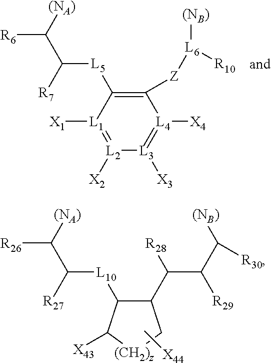

wherein (N_{A}) indicates the site of bonding of to NR₄ₐ of formula (I) and (N_{B}) indicates the site of bonding to NR_{4c} of formula (I);

R₁ is selected from the group consisting of —(CH₂)ₛCH₃, —CH(CH₃)(CH₂)ₜCH₃, —(CH₂)ᵤCH(CH₃)₂, —C(CH₃)₃, —CH₂—C(CH₃)₃, —CHR₁₇OR₁₈,

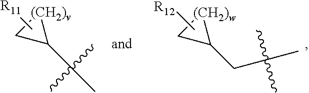

wherein s is 0, 1, 2, 3 or 4; t is 1, 2 or 3; u is 0, 1 or 2; v is 1, 2, 3 or 4; w is 1, 2, 3 or 4; and R₁₁ and R₁₂ are optionally present and, when present, are independently selected from the group consisting of C₁-C₄ alkyl, hydroxyl and alkoxy; R₁₇ is hydrogen or methyl; and R₁₈ is selected from the group consisting of hydrogen, C₁-C₄ alkyl and acyl;

R₂ₐ is selected from the group consisting of —CH₃, —CH₂CH₃, —CH(CH₃)₂, —CF₃, —CF₂H and —CH₂F;

R_{2b} is selected from the group consisting of —H and —CH₃;

R₃ₐ is selected from the group consisting of hydrogen, C₁-C₄ alkyl, hydroxyl and alkoxy;

R_{3b} is selected from the group consisting of hydrogen and C₁-C₄ alkyl;

R₄ₐ, R_{4b}, R_{4c} and R_{4d} are independently selected from the group consisting of hydrogen and C₁-C₄ alkyl;

R₅, when Y₁ is O or NR₁₆, is selected from the group consisting of hydrogen, C₁-C₄ alkyl and acyl; or, when Y₁ is C(═O), is selected from the group consisting of hydroxyl, alkoxy and amine;

R₆ is selected from the group consisting of hydrogen, C₁-C₄ alkyl, oxo and trifluoromethyl;

R₇ is selected from the group consisting of hydrogen, C₁-C₄ alkyl, hydroxyl, alkoxy and trifluoromethyl; or R₇ and X₁ together form a five or six-membered ring;

R₁₀ is selected from the group consisting of hydrogen, C₁-C₄ alkyl, 1,1,1-trifluoroethyl, hydroxyl and alkoxy, with the provisos that when L₆ is CH, R₁₀ is also selected from trifluoromethyl, and when L₆ is N, R₁₀ is also selected from sulfonyl; or R₁₀ and R₈ₐ together form a five- or six-membered ring;

R₂₆, R₂₈ and R₂₉ are independently selected from the group consisting of hydrogen, C₁-C₄ alkyl, hydroxyl, alkoxy and trifluoromethyl; or R₂₈ and R₂₉ together form a three-membered ring;

R₂₇ is selected from the group consisting of hydrogen, C₁-C₄ alkyl, hydroxyl, alkoxy and trifluoromethyl; or R₂₇ and X₄₃ together form a five or six-membered ring

R₃₀ is selected from the group consisting of hydrogen, C₁-C₄ alkyl, hydroxyl, alkoxy and trifluoromethyl;

Ar is selected from the group consisting of:

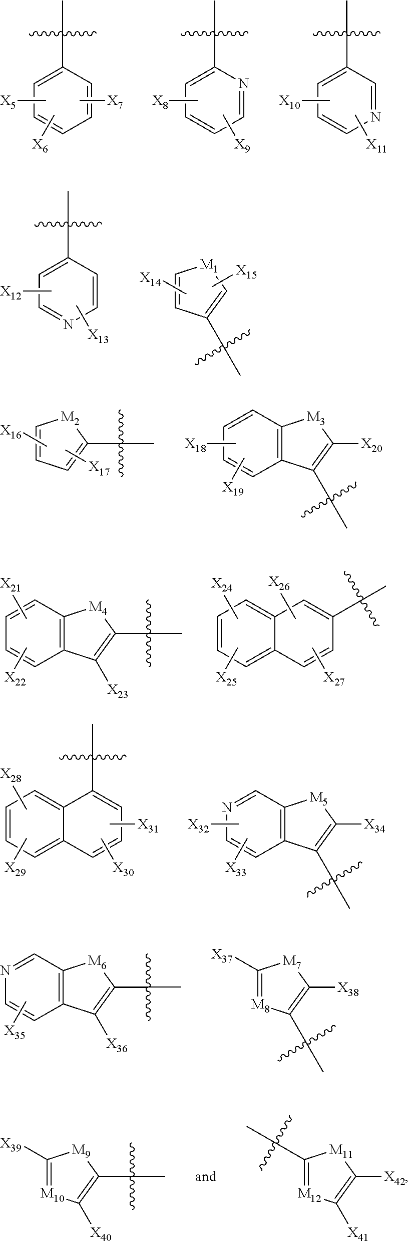

wherein M₁, M₂, M₃, M₄, M₅, M₆, M₇, M₉ and M₁₁ are independently selected from the group consisting of O, S and NR₁₃, wherein R₁₃ is selected from the group consisting of hydrogen, C₁-C₄ alkyl, formyl, acyl and sulfonyl; M₈, M₁₀ and M₁₂ are independently selected from the group consisting of N and CR₁₄, wherein R₁₄ is selected from the group consisting of hydrogen and C₁-C₄ alkyl; X₅, X₆, X₇, X₁₈, X₁₉, X₂₁, X₂₂, X₂₄, X₂₅, X₂₆, X₂₇, X₂₈, X₂₉, X₃₀ and X₃₁ are independently selected from the group consisting of hydrogen, halogen, trifluoromethyl and C₁-C₄ alkyl; and X₈, X₉, X₁₀, X₁₁, X₁₂, X₁₃, X₁₄, X₁₅, X₁₆, X₁₇, X₂₀, X₂₃, X₃₂, X₃₃, X₃₄, X₃₅, X₃₆, X₃₇, X₃₈, X₃₉, X₄₀, X₄₁ and X₄₂ are independently selected from the group consisting of hydrogen, hydroxyl, alkoxy, amino, halogen, cyano, trifluoromethyl and C₁-C₄ alkyl;

L₁, L₂, L₃, L₄ and L₆ are independently selected from the group consisting of CH and N;

L₅ is selected from the group consisting of CR₁₅ₐR_{15b}, O and NR_{15c}, wherein R₁₅ₐ and R_{15b} are independently selected from hydrogen, C₁-C₄ alkyl, hydroxyl and alkoxy; and R_{15c} is selected from the group consisting of hydrogen, C₁-C₄ alkyl, acyl and sulfonyl;

L₁₀ is selected from the group consisting of CR₃₅ₐR_{35b}, O and OC(═O)O, wherein R₃₅ₐ and R_{35b} are independently selected from hydrogen, C₁-C₄ alkyl, hydroxyl and alkoxy;

X₁ is selected from the group consisting of hydrogen, halogen, trifluoromethyl and C₁-C₄ alkyl; or X₁ and R₇ together form a five or six-membered ring;

X₂, X₃ and X₄ are independently selected from the group consisting of hydrogen, halogen, trifluoromethyl and C₁-C₄ alkyl;

X₄₃ and X₄₄ are optionally present and, when present, are independently selected from the group consisting of C₁-C₄ alkyl, hydroxyl, alkoxy and trifluoromethyl; or X₄₃ and R₂₇ together form a five or six-membered ring; and

Y₁ is selected from the group consisting of C(═O), O and NR₁₆, wherein R₁₆ is selected from the group consisting of hydrogen; C₁-C₄ alkyl, acyl and sulfonyl;

z is 0, 1, 2 or 3; and

Z is selected from the group consisting of (Ar)-CHR₈ₐCHR₉ₐ-(L₆), (Ar)-CR_{8b}═CR_{9b}-(L₆) and -(Ar)-C≡C-(L₆), wherein (Ar) indicates the site of bonding to the phenyl ring and (L₆) the site of bonding to L₆, R₈ₐ and R₉ₐ are independently selected from the group consisting of hydrogen, C₁-C₄ alkyl, hydroxyl, alkoxy, oxo and trifluoromethyl; R_{8b} and R_{9b} are independently selected from the group consisting of hydrogen, C₁-C₄ alkyl, fluoro, hydroxyl, alkoxy and trifluoromethyl; or R₈ₐ and R₉ₐ together form a three-membered ring; or R₈ₐ and R₁₀ together form a five- or six-membered ring; or R₈ₐ and X₄ together form a five- or six-membered ring; or R₉ₐ and X₄ together form a five- or six-membered ring; or R_{8b} and X₄ together form a five- or six-membered ring; or R_{9b} and X₄ together form a five- or six-membered ring.

Specific embodiments of the present invention provide for compounds of formula (I) with the structure:

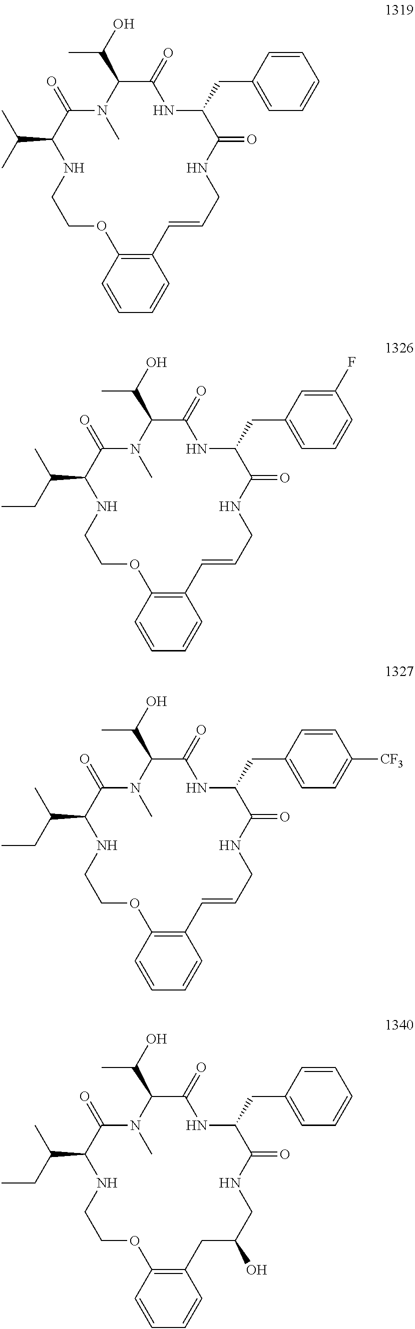

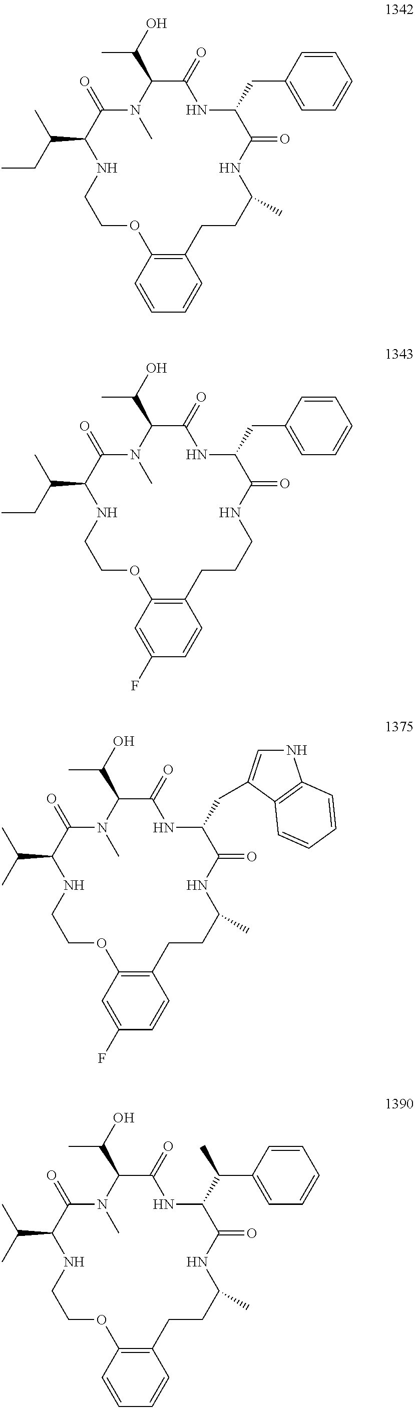

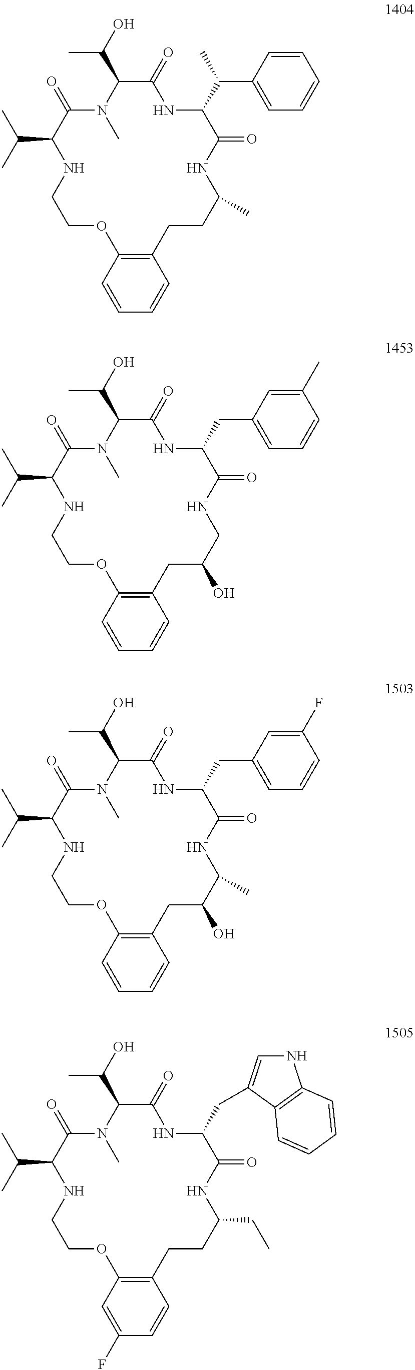

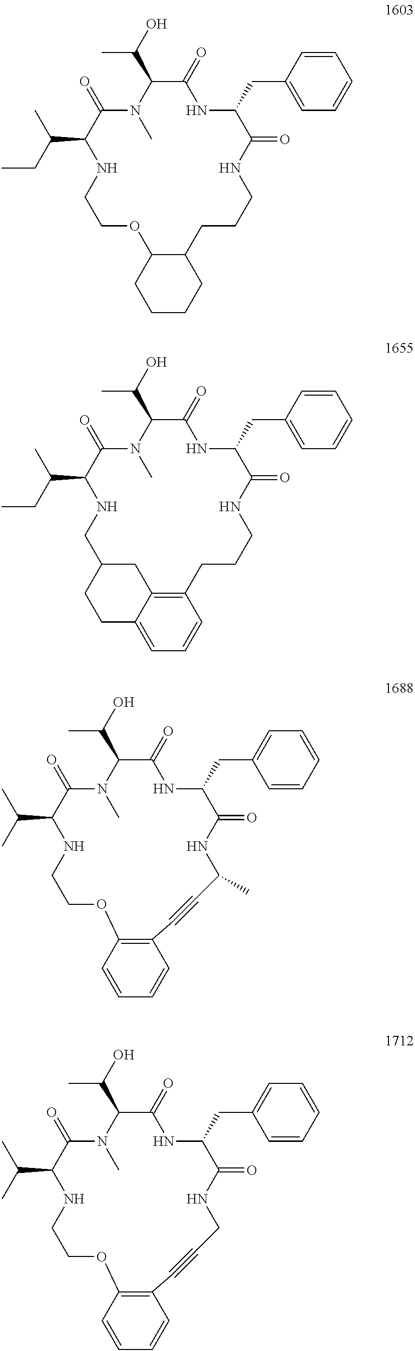

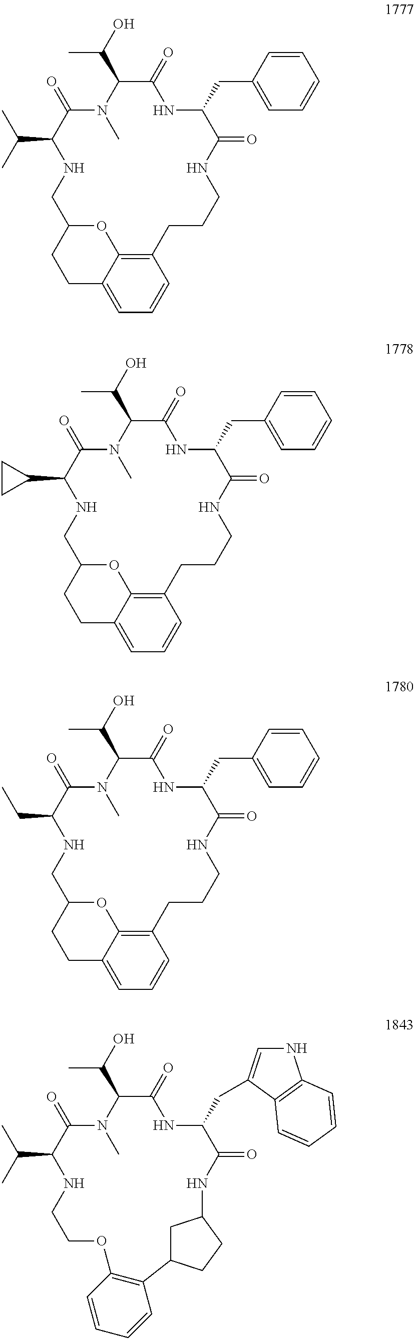

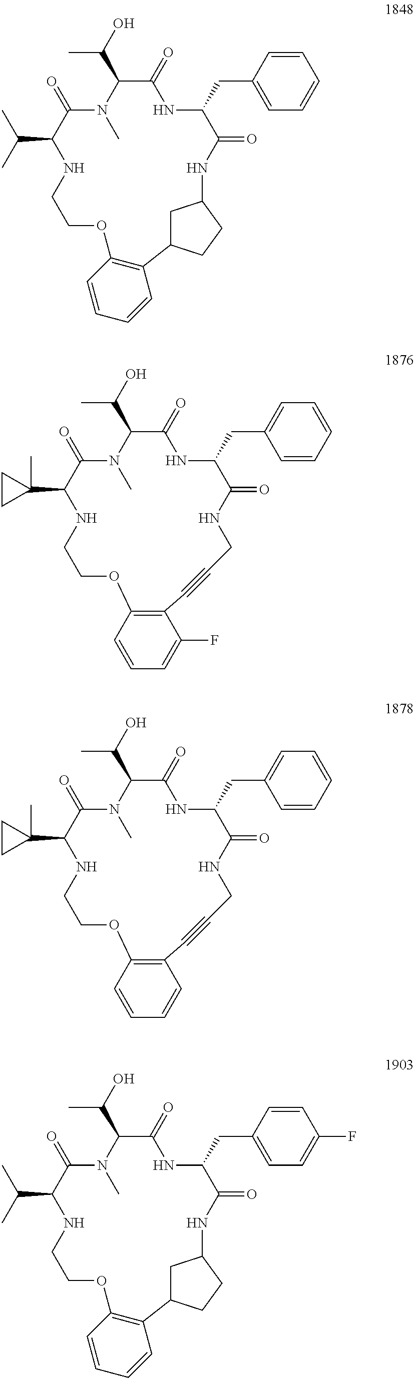

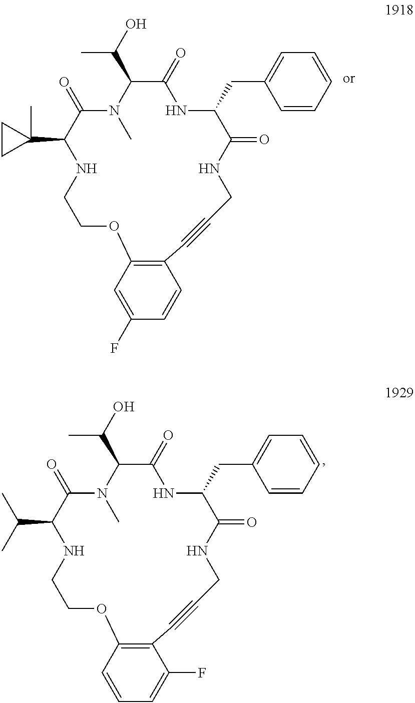

or a pharmaceutically acceptable salt thereof.

Further aspects of the present invention provide pharmaceutical compositions comprising: (a) a compound of the present invention; and (b) a pharmaceutically acceptable carrier, excipient or diluent.

In other aspects of the present invention, pharmaceutical compositions are provided comprising (a) a compound of the present invention; (b) one or more additional therapeutic agents; and (c) a pharmaceutically acceptable carrier, excipient or diluent.

For specific embodiments, the additional therapeutic agent is selected from the group comprising a GLP-1 agonist, a DPP-IV inhibitor, an amylin agonist, a PPAR-α agonist, a PPAR-γ agonist, a PPAR-α/γ dual agonist, a GDIR or GPR119 agonist, a PTP-1B inhibitor, a peptide YY agonist, an 11β-hydroxysteroid dehydrogenase (11β-HSD)-1 inhibitor, a sodium-dependent renal glucose transporter type 2 (SGLT-2) inhibitor, a glucagon antagonist, a glucokinase activator, an α-glucosidase inhibitor, a glucocorticoid antagonist, a glycogen synthase kinase 3β (GSK-3β) inhibitor, a glycogen phosphorylase inhibitor, an AMP-activated protein kinase (AMPK) activator, a fructose-1,6-biphosphatase inhibitor, a sulfonyl urea receptor antagonist, a retinoid X receptor activator, a 5-HT₁ₐ agonist, a 5-HT_{2c} agonist, a 5-HT₆ antagonist, a cannabioid antagonist or inverse agonist, a melanin concentrating hormone-1 (MCH-1) antagonist, a melanocortin-4 (MC4) agonist, a leptin agonist, a retinoic acid receptor agonist, a stearoyl-CoA desaturase-1 (SCD-1) inhibitor, a neuropeptide Y Y2 receptor agonist, a neuropeptide Y Y4 receptor agonist, a neuropeptide Y Y5 receptor antagonist, a neuronal nicotinic receptor α₄β₂ agonist a diacylglycerol acyltransferase 1 (DGAT-1) inhibitor, a thyroid receptor agonist, a lipase inhibitor, a fatty acid synthase inhibitor, a glycerol-3-phosphate acyltransferase inhibitor, a CPT-1 stimulant, an α_{1A}-adrenergic receptor agonist, an α_{2A}-adrenergic receptor agonist, a β₃-adrenergic receptor agonist, a histamine H3 receptor antagonist, a cholecystokinin A receptor agonist and a GABA-A agonist.

Additional aspects of the present invention provide kits comprising one or more containers containing pharmaceutical dosage units comprising an effective amount of one or more compounds of the present invention packaged with optional instructions for the use thereof.

In further aspects, the present invention provides methods of modulating GRLN receptor activity in a mammal comprising administering an effective GRLN receptor activity modulating amount of a compound of the present invention. According to some aspects of the present invention, the compound is a ghrelin receptor antagonist or a GRLN receptor antagonist. In yet another aspect, the compound is a ghrelin receptor inverse agonist or a GRLN receptor inverse agonist. According to another aspect of the present invention, the compound is both a ghrelin receptor antagonist and a ghrelin receptor inverse agonist or a GRLN receptor antagonist and a GRLN receptor inverse agonist.

Aspects of the present invention further relate to methods of preventing and/or treating disorders such as metabolic and/or endocrine disorders, obesity and obesity-associated disorders, appetite or eating disorders, addictive disorders, cardiovascular disorders, genetic disorders, hyperproliferative disorders, central nervous system disorders and inflammatory disorders.

In particular embodiments, the metabolic disorder is obesity, diabetes, metabolic syndrome, non-alcoholic fatty acid liver disease (NAFLD), non-alcoholic steatohepatitis (NASH) or steatosis.

In another specific embodiment, the appetite or eating disorder is Prader-Willi syndrome or hyperphagia.

In still other specific embodiments, the addictive disorder is alcohol dependendence, drug dependence or chemical dependence.

Further aspects of the present invention relate to methods of making the compounds of formula 1.

The present invention also relates to compounds of formula I useful for the preparation of a medicament for prevention and/or treatment of the disorders described herein.

Provided in a further embodiment is a macrocyclic compound selected from the group consisting of

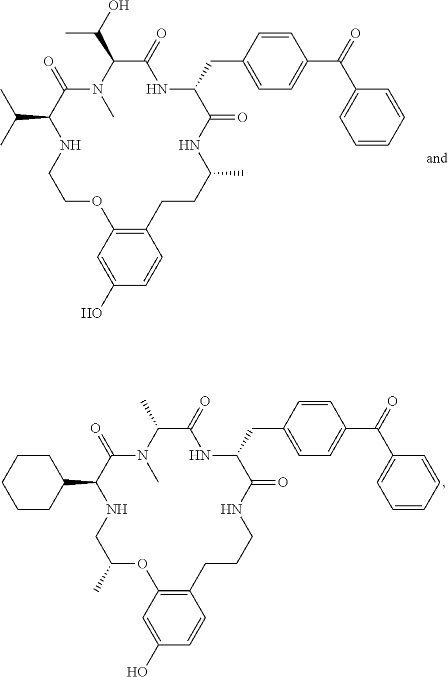

or a pharmaceutically acceptable salt thereof.

The foregoing and other aspects of the present invention are explained in greater detail in the specification set forth below.

### BRIEF DESCRIPTION OF THE DRAWINGS

FIG. 1 shows a chemical synthesis scheme for an exemplary compound of the present invention, compound 1319.

FIG. 2 shows a chemical synthesis scheme for an exemplary compound of the present invention, compound 1350.

FIG. 3 shows a chemical synthesis scheme for an exemplary compound of the present invention, compound 1636.

FIG. 4 shows a chemical synthesis scheme for an exemplary compound of the present invention, compound 1383.

FIG. 5 shows a chemical synthesis scheme for an exemplary compound of the present invention, compound 1390.

FIG. 6 shows a chemical synthesis scheme for an exemplary compound of the present invention, compound 1401.

FIG. 7 shows a chemical synthesis scheme for an exemplary compound of the present invention, compound 1300.

FIG. 8 shows a chemical synthesis scheme for an exemplary compound of the present invention, compound 1505.

FIG. 9 shows a graph presenting results of a study to assess the in vivo activity of an exemplary compound of the present invention, compound 1505, specifically the effect on body weight in the Zucker fatty rat model.

FIG. 10 shows a graph presenting results of a study to assess the in vivo activity of an exemplary compound of the present invention, compound 1505, specifically the effect on cumulative food consumption in the Zucker fatty rat model.

FIG. 11 shows a graph presenting results of a study to assess the in vivo activity of an exemplary compound of the present invention, compound 1712, specifically the effect on acute cumulative food consumption in the ob/ob mouse model.

FIG. 12 shows a graph presenting results of a study to assess the in vivo activity of an exemplary compound of the present invention, compound 1848, specifically the effect on cumulative food consumption in the ob/ob mouse model.

FIG. 13 shows a series of graphs presenting results of a study to assess the in vivo activity of an exemplary compound of the present invention, compound 1848, specifically the effect on selected metabolism parameters.

### DETAILED DESCRIPTION

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. Additionally, as used herein, the term “and/or” includes any and all combinations of one or more of the associated listed items and may be abbreviated as “/”.

Unless otherwise defined, all technical and scientific terms used herein have the same meaning as commonly understood by one of ordinary skill in the art to which this invention belongs.

All publications, U.S. patent applications, U.S. patents and other references cited herein are incorporated by reference in their entireties.

The term “alkyl” refers to straight or branched chain saturated or partially unsaturated hydrocarbon groups having from 1 to 20 carbon atoms, and in some instances, 1 to 8 carbon atoms. The term “lower alkyl” refers to alkyl groups containing 1 to 6 carbon atoms. Examples of alkyl groups include, but are not limited to, methyl, ethyl, isopropyl, tert-butyl, 3-hexenyl, and 2-butynyl. By “unsaturated” is meant the presence of 1, 2 or 3 double or triple bonds, or a combination of the two. Such alkyl groups may also be optionally substituted as described below.

When a subscript is used with reference to an alkyl or other hydrocarbon group defined herein, the subscript refers to the number of carbon atoms that the group may contain. For example, C₂-C₄ alkyl indicates an alkyl group that contains 2, 3 or 4 carbon atoms.

The term “cycloalkyl” refers to saturated or partially unsaturated cyclic hydrocarbon groups having from 3 to 15 carbon atoms in the ring, and in some instances, 3 to 7, and to alkyl groups containing said cyclic hydrocarbon groups. Examples of cycloalkyl groups include, but are not limited to, cyclopropyl, cyclopropylmethyl, cyclopentyl, 2-(cyclohexyl)ethyl, cycloheptyl, and cyclohexenyl. Cycloalkyl as defined herein also includes groups with multiple carbon rings, each of which may be saturated or partially unsaturated, for example decalinyl, [2.2.1]-bicycloheptanyl or adamantanyl. All such cycloalkyl groups may also be optionally substituted as described below.

The term “aromatic” refers to an unsaturated cyclic hydrocarbon group having a conjugated pi electron system that contains 4n+2 electrons where n is an integer greater than or equal to 1. Aromatic molecules are typically stable and are depicted as a planar ring of atoms with resonance structures that consist of alternating double and single bonds, for example benzene or naphthalene.

The term “aryl” refers to an aromatic group in a single or fused carbocyclic ring system having from 6 to 15 ring atoms, and in some instances, 6 to 10, and to alkyl groups containing said aromatic groups. Examples of aryl groups include, but are not limited to, phenyl, 1-naphthyl, 2-naphthyl and benzyl. Aryl as defined herein also includes groups with multiple aryl rings which may be fused, as in naphthyl and anthracenyl, or unfused, as in biphenyl and terphenyl. Aryl also refers to bicyclic or tricyclic carbon rings, where one of the rings is aromatic and the others of which may be saturated, partially unsaturated or aromatic, for example, indanyl or tetrahydronaphthyl (tetralinyl). All such aryl groups may also be optionally substituted as described below.

The term “heterocycle” or “heterocyclic” refers to saturated or partially unsaturated monocyclic, bicyclic or tricyclic groups having from 3 to 15 atoms, and in some instances, 3 to 7, with at least one heteroatom in at least one of the rings, said heteroatom being selected from O, S or N. Each ring of the heterocyclic group can contain one or two O atoms, one or two S atoms, one to four N atoms, provided that the total number of heteroatoms in each ring is four or leis and each ring contains at least one carbon atom. The fused rings completing the bicyclic or tricyclic heterocyclic groups may contain only carbon atoms and may be saturated or partially unsaturated. The N and S atoms may optionally be oxidized and the N atoms may optionally be quaternized. Heterocyclic also refers to alkyl groups containing said monocyclic, bicyclic or tricyclic heterocyclic groups. Examples of heterocyclic rings include, but are not limited to, 2- or 3-piperidinyl, 2- or 3-piperazinyl, 2- or 3-morpholinyl. All such heterocyclic groups may also be optionally substituted as described below

The term “heteroaryl” refers to an aromatic group in a single or fused ring system having from 5 to 15 ring atoms, and in some instances, 5 to 10, which have at least one heteroatom in at least one of the rings, said heteroatom being selected from O, S or N. Each ring of the heteroaryl group can contain one or two O atoms, one or two S atoms, one to four N atoms, provided that the total number of heteroatoms in each ring is four or less and each ring contains at least one carbon atom. The fused rings completing the bicyclic or tricyclic groups may contain only carbon atoms and may be saturated, partially unsaturated or aromatic. In structures where the lone pair of electrons of a nitrogen atom is not involved in completing the aromatic pi electron system, the N atoms may optionally be quaternized or oxidized to the N-oxide. Heteroaryl also refers to alkyl groups containing said cyclic groups. Examples of monocyclic heteroaryl groups include, but are not limited to pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, and triazinyl. Examples of bicyclic heteroaryl groups include, but are not limited to indolyl, benzothiazolyl, benzoxazolyl, benzothienyl, quinolinyl, tetrahydroisoquinolinyl, isoquinolinyl, benzimidazolyl, benzopyranyl, indolizinyl, benzofuranyl, isobenzofuranyl, chromonyl, coumarinyl, benzopyranyl, cinnolinyl, quinoxalinyl, indazolyl, purinyl, pyrrolopyridinyl, furopyridinyl, thienopyridinyl, dihydroisoindolyl, and tetrahydroquinolinyl. Examples of tricyclic heteroaryl groups include, but are not limited to carbazolyl, benzindolyl, phenanthrollinyl, acridinyl, phenanthridinyl, and xanthenyl. All such heteroaryl groups may also be optionally substituted as described below.

The term “hydroxyl” refers to the group —OH.

The term “alkoxy” refers to the group —ORₐ, wherein Rₐ is alkyl, cycloalkyl or heterocyclic. Examples include, but are not limited to methoxy, ethoxy, tert-butoxy, cyclohexyloxy and tetrahydropyranyloxy.

The term “aryloxy” refers to the group —OR_{b} wherein R_{b} is aryl or heteroaryl.

Examples include, but are not limited to phenoxy, benzyloxy and 2-naphthyloxy.

The term “acyl” refers to the group —C(═O)—R_{c}, wherein R_{c} is alkyl, cycloalkyl, heterocyclic, aryl or heteroaryl. Examples include, but are not limited to, acetyl, benzoyl and furoyl.

The term “amino acyl” indicates an acyl group that is derived from an amino acid.

The term “amino” refers to an —NR_{d}Rₑ group wherein R_{d} and Rₑ are independently selected from the group consisting of hydrogen, alkyl, cycloalkyl, heterocyclic, aryl and heteroaryl. Alternatively, R_{d} and Rₑ together form a heterocyclic ring of 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “amido” refers to the group —C(═O)—NR_{f}R_{g} wherein R_{f} and R_{g} are independently selected from the group consisting of hydrogen, alkyl, cycloalkyl, heterocyclic, aryl and heteroaryl. Alternatively, R_{f} and R_{g} together form a heterocyclic ring of 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “amidino” refers to the group —C(═NRₕ)NRᵢRⱼ wherein Rₕ is selected from the group consisting of hydrogen, alkyl, cycloalkyl, heterocyclic, aryl and heteroaryl; and Rᵢ and Rⱼ are independently selected from the group consisting of hydrogen, alkyl, cycloalkyl, heterocyclic, aryl and heteroaryl. Alternatively, Rᵢ and Rⱼ together form a heterocyclic ring of 3 to 8 members, optionally substituted with unsubstituted alkyl, unsubstituted cycloalkyl, unsubstituted heterocyclic, unsubstituted aryl, unsubstitutecl heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “carboxy” refers to the group —CO₂H.

The term “carboxyalkyl” refers to the group —CO₂Rₖ, wherein Rₖ is alkyl, cycloalkyl or heterocyclic.

The term “carboxyaryl” refers to the group —CO₂Rₘ, wherein Rₘ is aryl or heteroaryl.

The term “cyano” refers to the group —CN.

The term “formyl” refers to the group —C(═O)H, also denoted —CHO.

The term “halo,” “halogen” or “halide” refers to fluoro, fluorine or fluoride, chloro, chlorine or chloride, bromo, bromine or bromide, and iodo, iodine or iodide, respectively.

The term “oxo” refers to the bivalent group ═O, which is substituted in place of two hydrogen atoms on the same carbon to form a carbonyl group.

The term “mercapto” refers to the group —SRₙ wherein Rₙ is hydrogen, alkyl, cycloalkyl, heterocyclic, aryl or heteroaryl.

The term “nitro” refers to the group —NO₂.

The term “trifluoromethyl” refers to the group —CF₃.

The term “sulfinyl” refers to the group —S(═O)Rₚ wherein Rₚ is alkyl, cycloalkyl, heterocyclic, aryl or heteroaryl.

The term “sulfonyl” refers to the group —S(═O)₂—R_{q1} wherein R_{q1} is alkyl, cycloalkyl, heterocyclic, aryl or heteroaryl.

The term “aminosulfonyl” refers to the group —NR_{q2}—S(═O)₂—R_{q3} wherein R_{q2} is hydrogen, alkyl, cycloalkyl, heterocyclic, aryl or heteroaryl; and Rₒ is alkyl, cycloalkyl, heterocyclic, aryl or heteroaryl.

The term “sulfonamido” refers to the group —S(═O)₂—NRᵣRₛ wherein Rᵣ and Rₛ are independently selected from the group consisting of hydrogen, alkyl, cycloalkyl, heterocyclic, aryl or heteroaryl. Alternatively, Rᵣ and Rₛ together form a heterocyclic ring of 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “carbamoyl” refers to a group of the formula —N(Rₜ)—C(═O)—ORᵤ wherein Rₜ is selected from hydrogen, alkyl, cycloalkyl, heterocyclic, aryl or heteroaryl; and Rᵤ is selected from alkyl, cycloalkyl, heterocylic, aryl or heteroaryl.

The term “guanidino” refers to a group of the formula —N(Rᵥ)—C(═NR_{w})—NRₓR_{y} wherein Rᵥ, R_{w}, Rₓ and R_{y} are independently selected from hydrogen, alkyl, cycloalkyl, heterocyclic, aryl or heteroaryl. Alternatively, Rₓ and R_{y} together form a heterocyclic ring or 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “ureido” refers to a group of the formula —N(R_{z})—C(═O)—NRₐₐR_{bb} wherein R_{z}, Rₐₐ and R_{bb} are independently selected from hydrogen, alkyl, cycloalkyl, heterocyclic, aryl or heteroaryl. Alternatively, Rₐₐ and R_{bb} together with the nitrogen atom to which they are each bonded form a heterocyclic ring of 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “optionally substituted” is intended to expressly indicate that the specified group is unsubstituted or substituted by one or more suitable substituents, unless the optional substituents are expressly specified, in which case the term indicates that the group is unsubstituted or substituted with the specified substituents. As defined above, various groups may be unsubstituted or substituted (i.e., they are optionally substituted) unless indicated otherwise herein (e.g., by indicating that the specified group is unsubstituted).

The term “substituted” when used with the terms alkyl, cycloalkyl, heterocyclic, aryl and heteroaryl refers to an alkyl, cycloalkyl, heterocyclic, aryl or heteroaryl group having one or more of the hydrogen atoms of the group replaced by substituents independently selected from unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, halo, oxo, mercapto, sulfinyl, sulfonyl, sulfonamido, amidino, carbamoyl, guanidino, ureido and groups of the formulas —NR_{cc}C(═O)R_{dd}, —NRₑₑC(═NR_{ff})R_{gg}, —OC(═O)NRₕₕRᵢᵢ, —OC(═O)Rⱼⱼ, —OC(═O)ORₖₖ, —NRₘₘSO₂Rₙₙ, or —NRₚₚSO₂NR_{qq}Rᵣᵣ, wherein R_{cc}, R_{dd}, Rₑₑ, R_{ff}, R_{gg}, Rₕₕ, Rᵢᵢ, Rⱼⱼ, Rₘₘ, Rₚₚ, R_{qq} and Rᵣᵣ are independently selected from hydrogen, unsubstituted alkyl, unsubstituted cycloalkyl, unsubstituted heterocyclic, unsubstituted aryl or unsubstituted heteroaryl; and wherein Rₖₖ and Rₙₙ are independently selected from unsubstituted alkyl, unsubstituted cycloalkyl, unsubstituted heterocyclic, unsubstituted aryl or unsubstituted heteroaryl. Alternatively, R_{gg} and Rₕₕ, Rⱼⱼ and Rₖₖ or Rₚₚ and R_{qq} together with the nitrogen atom to which they are each bonded form a heterocyclic ring of 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N. In addition, the term “substituted” for aryl and heteroaryl groups includes as an option having one of the hydrogen atoms of the group replaced by cyano, nitro or trifluoromethyl.

A substitution is made provided that any atom's normal valency is not exceeded and that the substitution results in a stable compound. Generally, when a substituted form of a group is present, such substituted group may not be further substituted or, if substituted, the substituent comprises only a limited number of substituted groups, for example 1, 2, 3 or 4 such substituents.

When any variable occurs more than one time in any constituent or in any formula herein, its definition on each occurrence is independent of its definition at every other occurrence. Also, combinations of substituents and/or variables are permissible only if such combinations result in stable compounds.

A “stable compound” or “stable structure” is meant to mean a compound that is sufficiently robust to survive isolation to a useful degree of purity and formulation into an efficacious therapeutic agent.

The term “amino acid” refers to the common natural (genetically encoded) or synthetic amino acids and common derivatives thereof, known to those skilled in the art. When applied to amino acids, “standard” or “proteinogenic” refers to the genetically encoded 20 amino acids in their natural configuration. Similarly, when applied to amino acids, “unnatural” or “unusual” refers to the wide selection of non-natural, rare or synthetic amino acids such as those described by Hunt, S. in Chemistry and Biochemistry of the Amino Acids, Barrett, G. C., Ed., Chapman and Hall: New York, 1985.

The term “residue” with reference to an amino acid or amino acid derivative refers to a group of the formula:

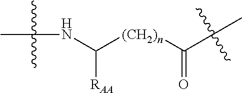

wherein R_{AA} is an amino acid side chain, and n=0, 1 or 2 in this instance.

The term “fragment” with respect to a dipeptide, tripeptide or higher order peptide derivative indicates a group that contains two, three or more, respectively, amino acid residues.

The term “amino acid side chain” refers to any side chain from a standard or unnatural amino acid, and is denoted R_{AA}. For example, the side chain of alanine is methyl, the side chain of valine is isopropyl and the side chain of tryptophan is 3-indolylmethyl.

The term “agonist” refers to a compound that duplicates at least some of the effect of the endogenous ligand of a protein, receptor, enzyme or the like.

The term “antagonist” refers to a compound that inhibits at least some of the effect of the endogenous ligand of a protein, receptor, enzyme or the like.

The term “inverse agonist” refers to a compound that decreases, at least to some degree, the baseline functional activity of a protein, receptor, enzyme or the like, such as the constitutive signaling activity of a G protein-coupled receptor or variant thereof. An inverse agonist can also be an antagonist.

The term “baseline functional activity” refers to the activity of a protein, receptor, enzyme or the like, including constitutive signaling activity, in the absence of the endogenous ligand.

The term “growth hormone secretagogue” (GHS) refers to any exogenously administered compound or agent that directly or indirectly stimulates or increases the endogenous release of growth hormone, growth hormone-releasing hormone, or somatostatin in an animal, in particular, a human. A GHS may be peptidic or non-peptidic in nature, with an agent that can be administered orally preferred. In addition, an agent that induces a pulsatile response is preferred.

The term “modulator” refers to a compound that imparts an effect on a biological or chemical process or mechanism. For example, a modulator may increase, facilitate, upregulate, activate, inhibit, decrease, block, prevent, delay, desensitize, deactivate, down regulate, or the like, a biological or chemical process or mechanism. Accordingly, a modulator can be an “agonist,” an “antagonist,” or an “inverse agonist.” Exemplary biological processes or mechanisms affected by a modulator include, but are not limited to, receptor binding and hormone release or secretion. Exemplary chemical processes or mechanisms affected by a modulator include, but are not limited to, catalysis and hydrolysis.

The term “variant” when applied to a receptor is meant to include dimers, trimers, tetramers, pentamers and other biological complexes containing multiple components. These components can be the same or different.

The term “peptide” refers to a chemical compound comprised of two or more amino acids covalently bonded together.

The term “peptidomimetic” refers to a chemical compound designed to mimic a peptide, but which contains structural differences through the addition or replacement of one of more functional groups of the peptide in order to modulate its activity or other properties, such as solubility, metabolic stability, oral bioavailability, lipophilicity, permeability, etc. This can include replacement of the peptide bond, side chain modifications, truncations, additions of functional groups, etc. When the chemical structure is not derived from the peptide, but mimics its activity, it is often referred to as a “non-peptide peptidomimetic.”

The term “peptide bond” refers to the amide [—C(═O)—NH—] functionality with which individual amino acids are typically covalently bonded to each other in a peptide.

The term “protecting group” refers to any chemical compound that may be used to prevent a potentially reactive functional group, such as an amine, a hydroxyl or a carboxyl, on a molecule from undergoing a chemical reaction while chemical change occurs elsewhere in the molecule. A number of such protecting groups are known to those skilled in the art and examples can be found in “Protective Groups in Organic Synthesis,” Theodora W. Greene and Peter G. Wuts, editors, John Wiley & Sons, New York, 3^{rd} edition, 1999 [ISBN 0471160199]. Examples of amino protecting groups include, but are not limited to, phthalimido, trichloroacetyl, benzyloxycarbonyl, tert-butoxycarbonyl, and adamantyloxy-carbonyl. Preferred amino protecting groups are carbamate amino protecting groups, which are defined as an amino protecting group that when bound to an amino group forms a carbamate. Preferred amino carbamate protecting groups are all ylox ylcarbonyl (Alloc or Aloe), benzyloxycarbonyl (Cbz), 9-fluorenylmethoxycarbonyl (Fmoc), tert-butoxycarbonyl (Boc) and α,α-dimethyl-3,5-dimethoxybenzyloxycarbonyl (Ddz). For a recent discussion of newer nitrogen protecting groups: Theodoridis, G. Tetrahedron 2000, 56, 2339-2358. Examples of hydroxyl protecting groups include, but are not limited to, acetyl, tert-butyldimethylsilyl (TBDMS), trityl (Trt), tert-butyl, and tetrahydropyranyl (THP). Examples of carboxyl protecting groups include, but are not limited to methyl ester, teri-butyl ester, benzyl ester, trimethylsilylethyl ester, and 2,2,2-trichloroethyl ester.

The term “solid phase chemistry” refers to the conduct of chemical reactions where one component of the reaction is covalently bonded to a polymeric material (solid support as defined below). Reaction methods for performing chemistry on solid phase have become more widely known and established outside the traditional fields of peptide and oligonucleotide chemistry.

The term “solid support,” “solid phase” or “resin” refers to a mechanically and chemically stable polymeric matrix utilized to conduct solid phase chemistry. This is denoted by “Resin,” “P-” or the following symbol:

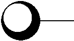

Examples of appropriate polymer materials include, but are not limited to, polystyrene, polyethylene, polyethylene glycol, polyethylene glycol grafted or covalently bonded to polystyrene (also termed PEG-polystyrene, TentaGelml, Rapp, W.; Zhang, L.; Bayer, E. In Innovations and Persepctives in Solid Phase Synthesis. Peptides, Polypeptides and Oligonucleotides; Epton, R., Ed.; SPCC Ltd.: Birmingham, UK; p 205), polyacrylate (CLEAR™), polyacrylamide, polyurethane, PEGA [polyethyleneglycol poly(N,N-dimethylacrylamide) co-polymer, Meldal, M. Tetrahedron Len. 1992, 33, 3077 3080], cellulose, etc. These materials can optionally contain additional chemical agents to form cross-linked bonds to mechanically stabilize the structure, for example polystyrene cross-linked with divinylbenezene (DVB, usually 0.1-5%, or 0.5-2%). This solid support can include as non-limiting examples aminomethyl polystyrene, hydroxymethyl polystyrene, benzhydrylamine polystyrene (BHA), methylbenzhydrylamine (MBHA) polystyrene, and other polymeric backbones containing free chemical functional groups, most typically, —NH, or —OH, for further derivatization or reaction. The term is also meant to include “Ultraresins” with a high proportion (“loading”) of these functional groups such as those prepared from polyethyleneimines and cross-linking molecules (Barth, M.; Rademann, J. J. Comb. Chem. 2004, 6, 340-349). At the conclusion of the synthesis, resins are typically discarded, although they have been shown to be able to be reused such as in Frechet, J. M. J.; Haque, K. E. Tetrahedron Lett. 1975, 16, 3055.

In general, the materials used as resins are insoluble polymers, but certain polymers have differential solubility depending on solvent and can also be employed for solid phase chemistry. For example, polyethylene glycol can be utilized in this manner since it is soluble in many organic solvents in which chemical reactions can be conducted, but it is insoluble in others, such as diethyl ether. Hence, reactions can be conducted homogeneously in solution, then the product on the polymer precipitated through the addition of diethyl ether and processed as a solid. This has been termed “liquid-phase” chemistry.

The term “linker” when used in reference to solid phase chemistry refers to a chemical group that is bonded covalently to a solid support and is attached between the support and the substrate typically in order to permit the release (cleavage) of the substrate from the solid support. However, it can also be used to impart stability to the bond to the solid support or merely as a spacer element. Many solid supports are available commercially with linkers already attached.

Abbreviations used for amino acids and designation of peptides follow the rules of the IUPAC-IUB Commission of Biochemical Nomenclature in J. Biol. Chem. 1972, 247, 977-983. This document has been updated: Biochem. J., 1984, 219, 345-373; Eur. J. Biochem., 1984, 138, 9-37; 1985, 152, 1; Int. J. Pept. Prot. Res., 1984, 24, following p 84; J. Biol. Chem., 1985, 260, 14-42; Pure Appl. Chem., 1984, 56, 595-624; Amino Acids and Peptides, 1985, 16, 387-410; and in Biochemical Nomenclature and Related Documents, 2nd edition, Portland Press, 1992, pp 39-67. Extensions to the rules were published in the JCBN/NC-IUB Newsletter 1985, 1986, 1989; see Biochemical Nomenclature and Related Documents, 2nd edition, Portland Press, 1992, pp 68-69.

The term “effective amount” or “effective” is intended to designate a dose that causes a relief of symptoms of a disease or disorder as noted through clinical testing and evaluation, patient observation, and the like, and/or a dose that causes a detectable change in biological or chemical activity as detected by one skilled in the art for the relevant mechanism or process. As is generally understood in the art, the dosage will vary depending on the administration routes, symptoms and body weight of the patient but also depending upon the compound being administered.

Administration of two or more compounds “in combination” means that the two compounds are administered closely enough in time that the presence of one alters the biological effects of the other. The two compounds can be administered simultaneously (concurrently) or sequentially. Simultaneous administration can be carried out by mixing the compounds prior to administration, or by administering the compounds at the same point in time but at different anatomic sites or using different routes of administration. The phrases “concurrent administration”, “administration in combination”, “simultaneous administration” or “administered simultaneously” as used herein, means that the compounds are administered at the same point in time or immediately following one another. In the latter case, the two compounds are administered at times sufficiently close that the results observed are indistinguishable from those achieved when the compounds are administered at the same point in time.

The term “pharmaceutically active metabolite” is intended to mean a pharmacologically active product produced through metabolism in the body of a specified compound.

The term “solvate” is intended to mean a pharmaceutically acceptable solvate form of a specified compound that retains the biological effectiveness of such compound. Examples of solvates, without limitation, include compounds of the invention in combination with water, isopropanol, ethanol, methanol, DMSO, ethyl acetate, acetic acid, or ethanolamine.

The macrocyclic compounds of the invention have been shown to possess ghrelin modulating activity, and in particular embodiments, as antagonists or inverse agonists. A series of macrocyclic peptidomimetics recently has been described as modulators of the ghrelin receptor and their uses for the treatment and prevention of a range of medical conditions including metabolic and/or endocrine disorders, gastrointestinal disorders, cardiovascular disorders, obesity and obesity-associated disorders, central nervous system disorders, genetic disorders, hyperproliferative disorders and inflammatory disorders outlined (U.S. Pat. Nos. 7,452,862, 7,476,653 and 7,491,695; Intl. Pat. Appl. Publ. Nos. WO 2006/009645, WO 2006/009674, WO 2006/046977, WO 2006/137974 and WO 2008/130464; U.S. Pat. Appl. Publ. Nos. 2006/025566, 2007/021331, 2008/051383 and 2008/194672). One of these compounds, TZP-101, a ghrelin agonist, has entered the clinic as a treatment for gastrointestinal dysmotility diorders. (Lasseter, K. C.; Shaughnessy, L.; Cummings, D.; et al. J. Clin. Pharmacol. 2008, 48, 193-202). The compounds of the present invention differ in structural composition and chiral configuration when compared to these agonists.

Although binding potency and target affinity are factors in drug discovery and development, also important for development of viable pharmaceutical agents are optimization of pharmacokinetic (PK) and/or pharmacodynamic (PD) parameters. A focus area for research in the pharmaceutical industry has been to better understand the underlying factors which determine the suitability of molecules in this manner, often colloquially termed its “drug-likeness.” (Lipinski, C. A.; Lombardo, F.; Dominy, B. W.; Feeney, P. J. Adv. Drug Delivery Rev. 1997, 23, 3-25; Muegge, I. Med. Res. Rev. 2003, 23, 302-321; Veber, D. F.; Johnson, S. R.; Cheng, H.-Y.; Smith, B. R.; Ward, K. W.; Kopple, K. D. J. Med. Chem. 2002, 45, 2615-2623.) For example, molecular weight, log P, membrane permeability, the number of hydrogen bond donors and acceptors, total polar surface area (TPSA), and the number of rotatable bonds have all been correlated with compounds that have been successful in drug development. Additionally, experimental measurements of plasma protein binding, interaction with cytochrome P450 enzymes, and pharmacokinetic parameters are employed in the pharmaceutical industry to select and advance new drug candidates.

However, these parameters have not been widely explored or reported within the macrocyclic structural class. This creates tremendous challenges in drug development for these molecules. The macrocyclic compounds of the present invention have been found to possess such desirable pharmacological characteristics, while maintaining sufficient binding affinity and/or selectivity for the ghrelin receptor, as illustrated in the Examples. These combined characteristics are superior to the macrocyclic ghrelin antagonist compounds previously described and make them more suitable for development as pharmaceutical agents, particularly for use as orally administered agents or for chronic uses.

### 1. Compounds

Novel macrocyclic compounds of the present invention include those of formula (I):

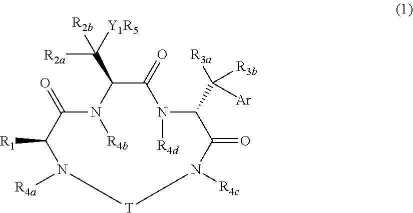

or a pharmaceutically acceptable salt thereof, wherein the component T is selected from

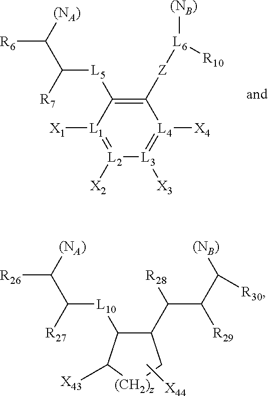

wherein (N_{A}) indicates the site of bonding of to NR₄ₐ of formula (1) and (N_{B}) indicates the site of bonding to NR_{4c} of formula (I);

In specific embodiments, the compound can have any of the structures defined in Table 1. These structures are based upon the structural formula (A):

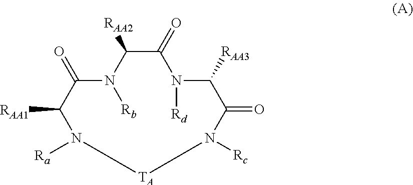

TABLE 1
Representative Compounds of the Invention

| Compound | R_{AA1} | R_{AA2} | R_{AA3} | T_{A} |
|---|---|---|---|---|
| 1300 | 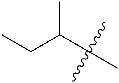 | 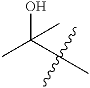 | 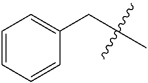 | T8 |
| 1301 | 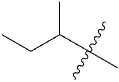 | 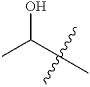 | 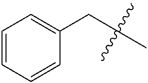 | T33a |
| 1302 | 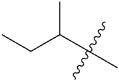 | 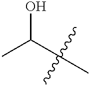 | 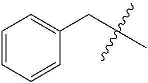 | T125b |
| 1304 | 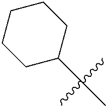 | 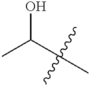 | 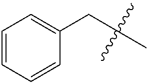 | T8 |
| 1305 | 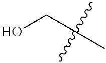 | 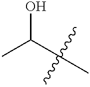 | 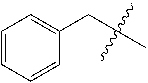 | T8 |
| 1311 | 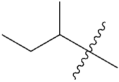 | 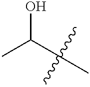 | 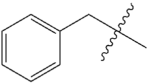 | T11 |
| 1313 | 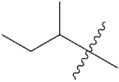 | 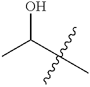 | 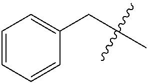 | T165a |
| 1314 | 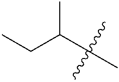 | 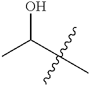 | 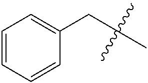 | T165b |
| 1315 | 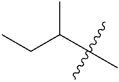 | 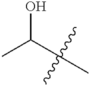 | 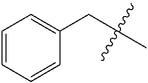 | T156a |
| 1316 | 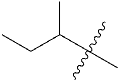 | 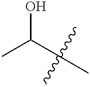 | 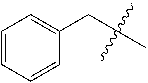 | T156b |
| 1317 | 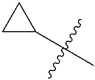 | 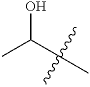 | 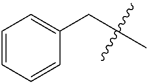 | T156b |
| 1318 | 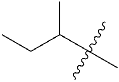 | 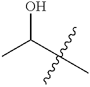 | 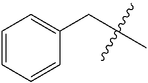 | T8 |
| 1319 | 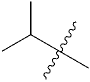 | 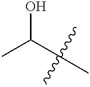 | 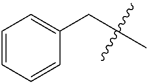 | T8 |
| 1320 | 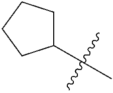 | 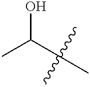 | 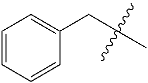 | T8 |
| 1323 | 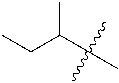 | 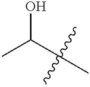 | 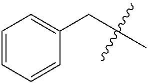 | T8 |
| 1324 | 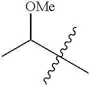 | 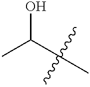 | 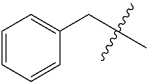 | T8 |
| 1325 | 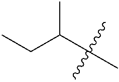 | 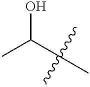 | 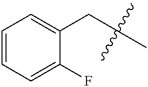 | T8 |
| 1326 | 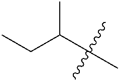 | 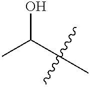 | 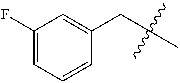 | T8 |
| 1327 | 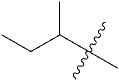 | 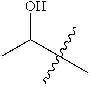 | 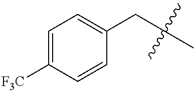 | T8 |
| 1328 | 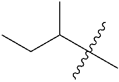 | 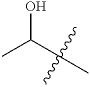 | 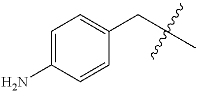 | T8 |
| 1329 | 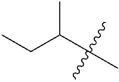 | 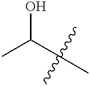 | 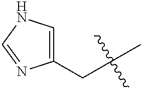 | T8 |
| 1330 | 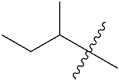 | 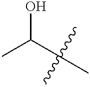 | 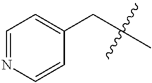 | T8 |
| 1331 | 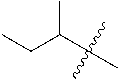 | 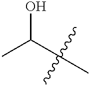 | 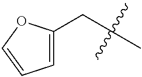 | T8 |
| 1332 | 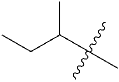 | 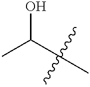 | 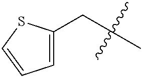 | T8 |
| 1333 | 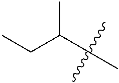 | 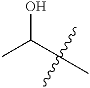 | 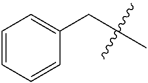 | T154 |
| 1334 | 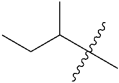 | 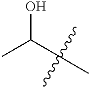 | 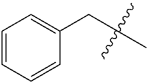 | T67 |
| 1335 | 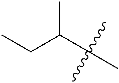 | 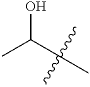 | 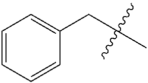 | T106 |
| 1336 | 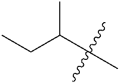 | 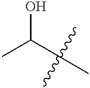 | 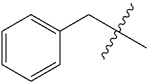 | T113a |
| 1337 | 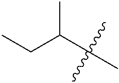 | 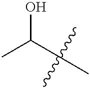 | 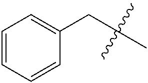 | T113b |
| 1338 | 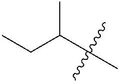 | 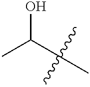 | 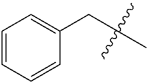 | T40 |
| 1339 | 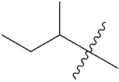 | 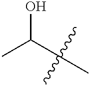 | 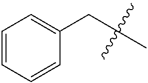 | T59a |
| 1340 | 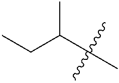 | 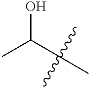 | 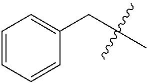 | T59b |
| 1341 | 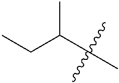 | 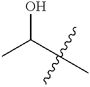 | 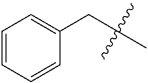 | T160 |
| 1342 | 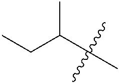 | 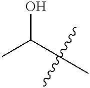 | 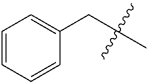 | T125a |
| 1343 | 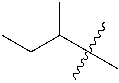 | 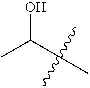 | 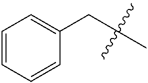 | T69 |
| 1344 | 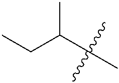 | 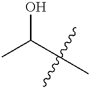 | 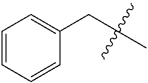 | T129b |
| 1345 | 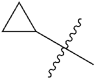 | 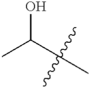 | 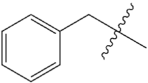 | T125b |
| 1346 | 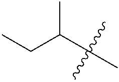 | 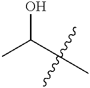 | 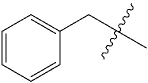 | T158 |
| 1347 | 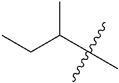 | 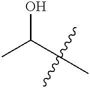 | 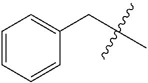 | T38a |
| 1348 | 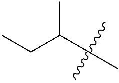 | 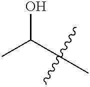 | 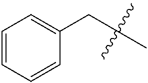 | T38b |
| 1349 | 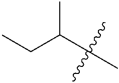 | 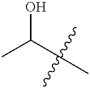 | 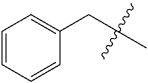 | T151 |
| 1350 | 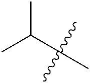 | 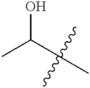 | 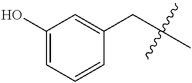 | T8 |
| 1351 | 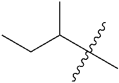 | 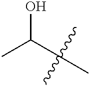 | 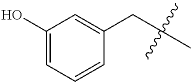 | T8 |
| 1352 | 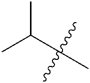 | 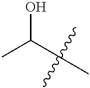 | 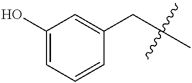 | T125a |
| 1353 | 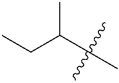 | 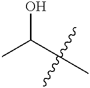 | 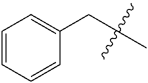 | T8 |
| 1354 | 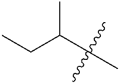 | 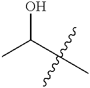 | 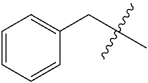 | T9 |
| 1355 | 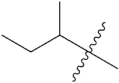 | 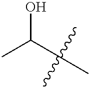 | 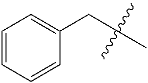 | T8 |
| 1356 | 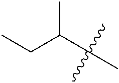 | 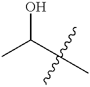 | 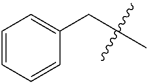 | T9 |
| 1357 | 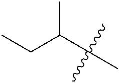 | 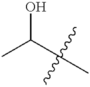 | 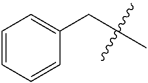 | T167 |
| 1358 | 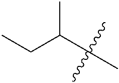 | 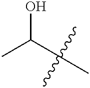 | 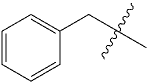 | T125a |
| 1359 | 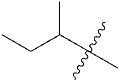 | 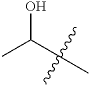 | 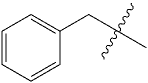 | T59b |
| 1360 | 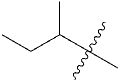 | 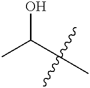 | 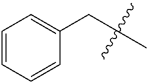 | T69 |
| 1361 | 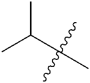 | 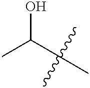 | 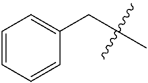 | T125a |
| 1362 | 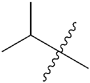 | 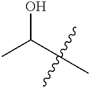 | 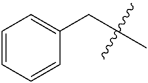 | T59b |
| 1363 | 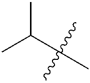 | 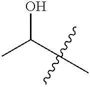 | 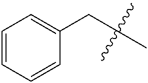 | T69 |
| 1364 | 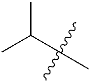 | 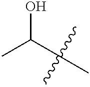 | 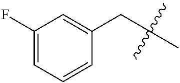 | T125a |
| 1365 | 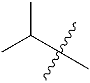 | 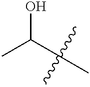 | 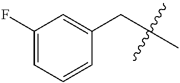 | T59b |
| 1366 | 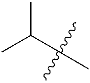 | 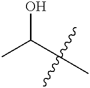 | 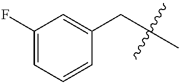 | T69 |
| 1367 | 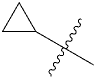 | 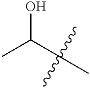 | 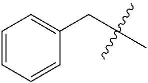 | T125a |
| 1368 | 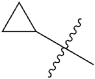 | 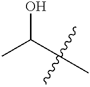 | 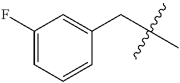 | T125a |
| 1369 | 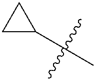 | 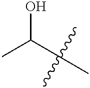 | 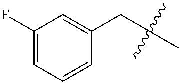 | T128a |
| 1370 | 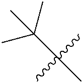 | 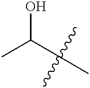 | 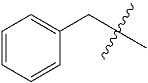 | T125a |
| 1371 | 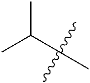 | 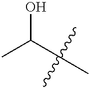 | 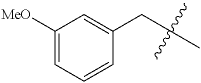 | T125a |
| 1372 | 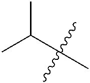 | 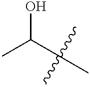 | 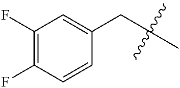 | T125a |
| 1373 | 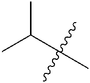 | 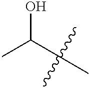 | 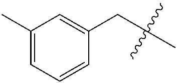 | T125a |
| 1374 | 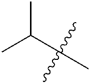 | 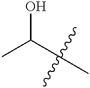 | 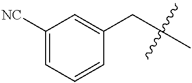 | T125a |
| 1375 | 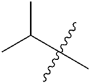 | 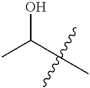 | 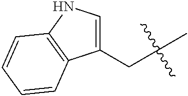 | T125a |
| 1376 | 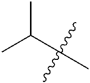 | 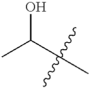 | 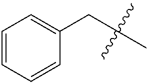 | T86 |
| 1377 | 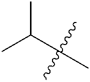 | 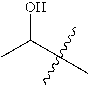 | 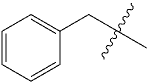 | T70 |
| 1378 | 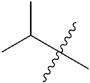 | 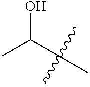 | 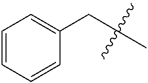 | T87 |
| 1379 | 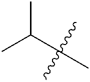 | 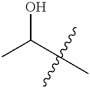 | 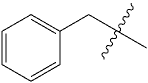 | T162a |
| 1380 | 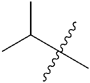 | 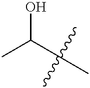 | 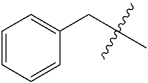 | T163a |
| 1381 | 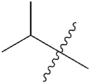 | 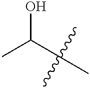 | 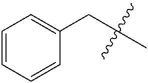 | T164a |
| 1382 | 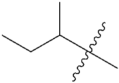 | 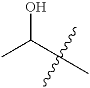 | 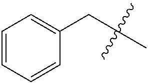 | T166 |
| 1383 | 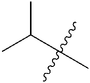 | 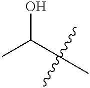 | 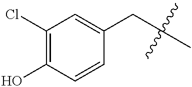 | T125a |
| 1384 | 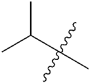 | 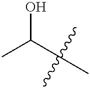 | 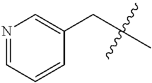 | T125a |
| 1385 | 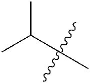 | 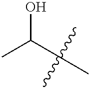 | 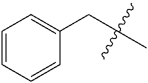 | T11 |
| 1387 | 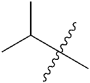 | 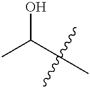 | 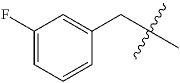 | T125a |
| 1388 | 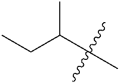 | 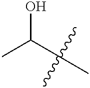 | 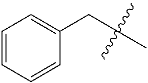 | T166 |
| 1389 | 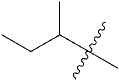 | 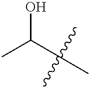 | 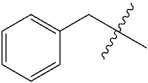 | T167 |
| 1390 | 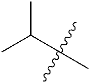 | 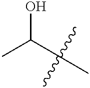 | 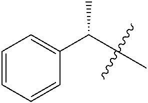 | T125a |
| 1391 | 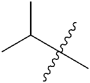 | 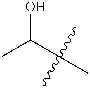 | 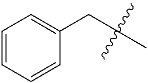 | T129a |
| 1392 | 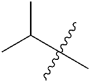 | 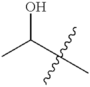 | 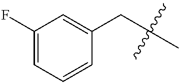 | T129a |
| 1393 | 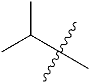 | 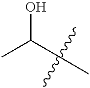 | 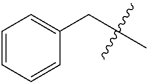 | T161a |
| 1394 | 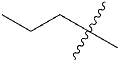 | 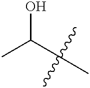 | 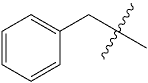 | T125a |
| 1395 | 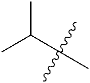 | 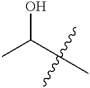 | 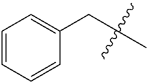 | T208a |
| 1396 | 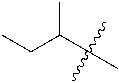 | 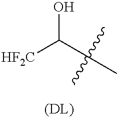 | 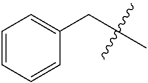 | T8 |
| 1397 | 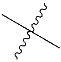 | 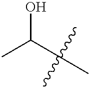 | 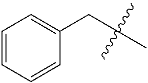 | T125a |
| 1398 | 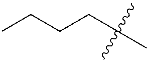 | 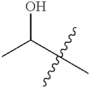 | 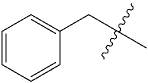 | T125a |
| 1399 | 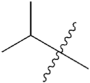 | 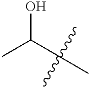 | 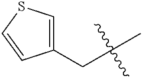 | T125a |
| 1400 | 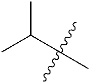 | 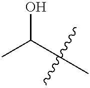 | 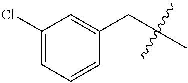 | T125a |
| 1401 | 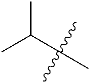 | 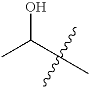 | 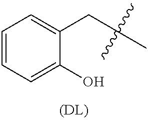 | T125a |
| 1402 | 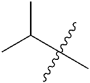 | 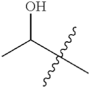 | 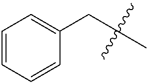 | T151b |
| 1403 | 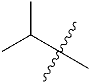 | 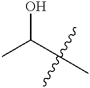 | 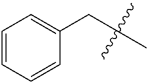 | T151a |
| 1404 | 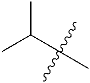 | 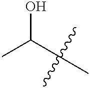 | 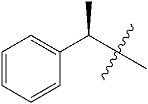 | T125a |
| 1405 | 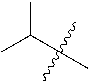 | 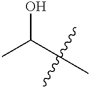 | 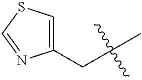 | T125a |
| 1406 | 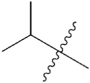 | 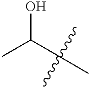 | 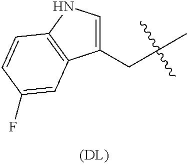 | T125a |
| 1407 | 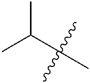 | 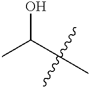 | 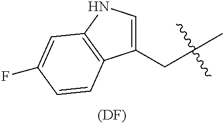 | T125a |
| 1408 | 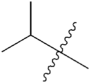 | 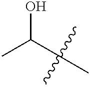 | 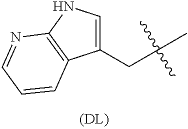 | T125a |
| 1409 | 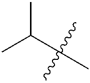 | 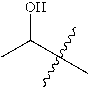 | 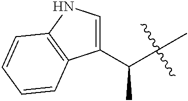 | T125a |
| 1411 | 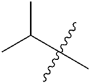 | 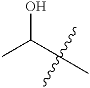 | 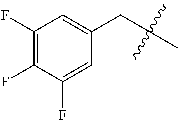 | T125a |
| 1412 | 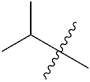 | 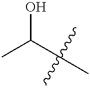 | 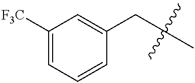 | T125a |
| 1413 | 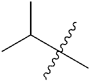 | 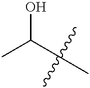 | 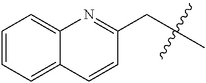 | T125a |
| 1414 | 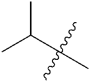 | 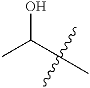 | 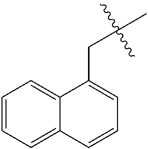 | T125a |
| 1415 | 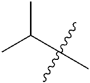 | 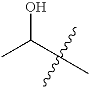 | 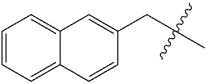 | T125a |
| 1416 | 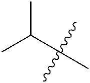 | 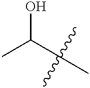 | 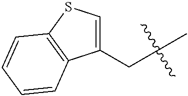 | T125a |
| 1417 | 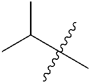 | 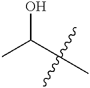 | 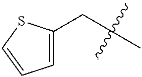 | T125a |
| 1418 | 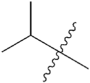 | 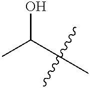 | 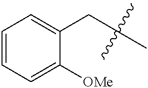 | T125a |
| 1419 | 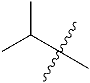 | 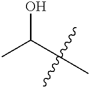 | 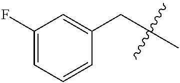 | T8 |
| 1420 | 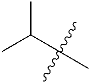 | 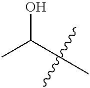 | 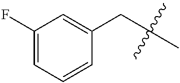 | T163a |
| 1421 | 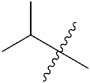 | 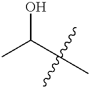 | 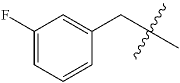 | T164a |
| 1422 | 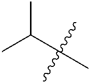 | 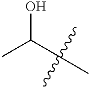 | 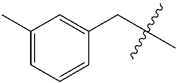 | T8 |
| 1423 | 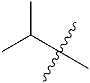 | 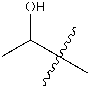 | 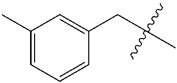 | T163a |
| 1424 | 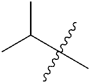 | 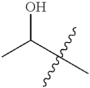 | 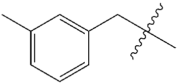 | T164a |
| 1425 | 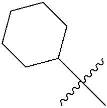 | 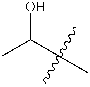 | 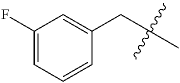 | T125a |
| 1426 | 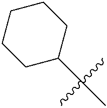 | 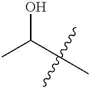 | 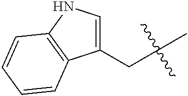 | T125a |
| 1427 | 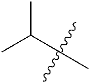 | 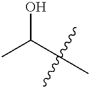 | 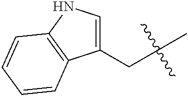 | T8 |
| 1428 | 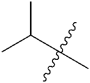 | 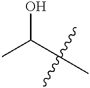 | 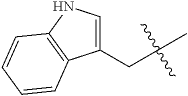 | T163a |
| 1429 | 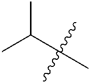 | 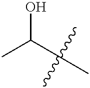 | 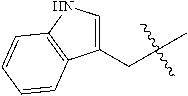 | T164a |
| 1430 | 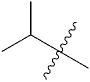 | 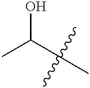 | 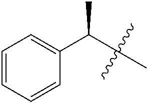 | T8 |
| 1431 | 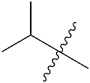 | 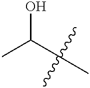 | 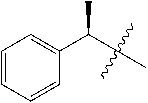 | T164a |
| 1432 | 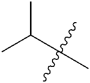 | 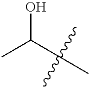 | 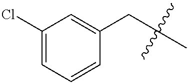 | T8 |
| 1433 | 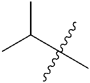 | 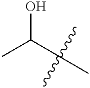 | 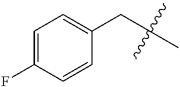 | T8 |
| 1434 | 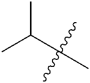 | 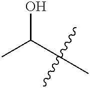 | 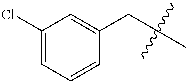 | T163a |
| 1435 | 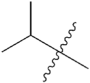 | 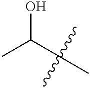 | 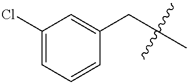 | T164a |
| 1436 | 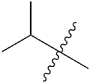 | 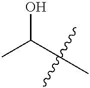 | 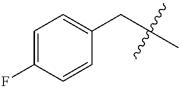 | T163a |
| 1437 | 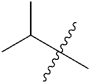 | 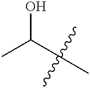 | 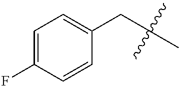 | T164a |
| 1438 | 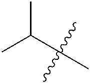 | 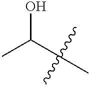 | 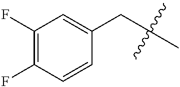 | T8 |
| 1439 | 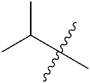 | 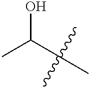 | 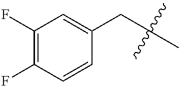 | T163a |
| 1440 | 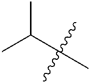 | 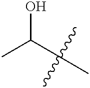 | 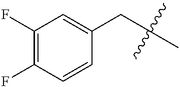 | T164a |
| 1441 | 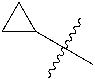 | 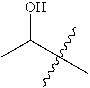 | 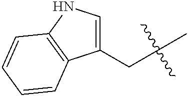 | T8 |
| 1442 | 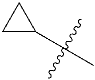 | 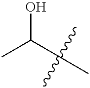 | 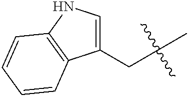 | T163a |
| 1443 | 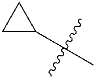 | 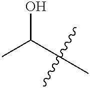 | 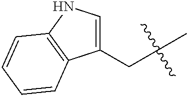 | T164a |
| 1444 | 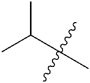 | 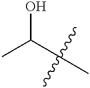 | 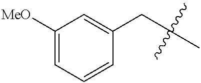 | T163a |
| 1445 | 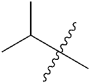 | 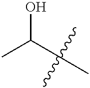 | 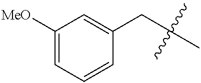 | T8 |
| 1446 | 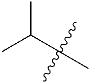 | 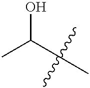 | 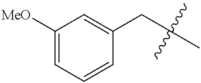 | T164a |
| 1447 | 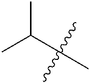 | 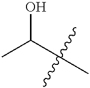 | 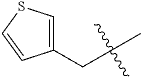 | T8 |
| 1448 | 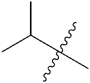 | 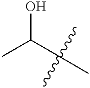 | 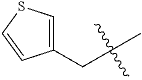 | T163a |
| 1449 | 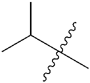 | 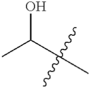 | 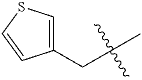 | T164a |
| 1450 | 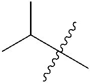 | 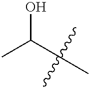 | 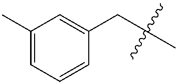 | T69 |
| 1451 | 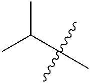 | 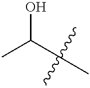 | 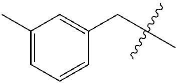 | T129a |
| 1453 | 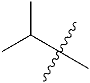 | 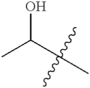 | 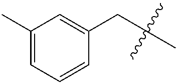 | T59b |
| 1454 | 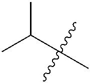 | 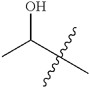 | 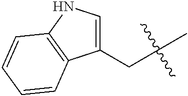 | T69 |
| 1455 | 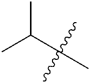 | 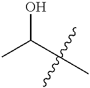 | 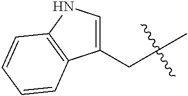 | T129a |
| 1456 | 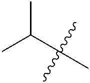 | 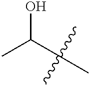 | 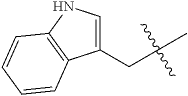 | T59b |
| 1457 | 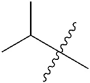 | 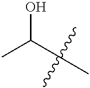 | 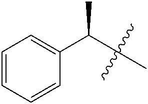 | T69 |
| 1458 | 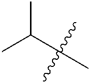 | 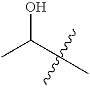 | 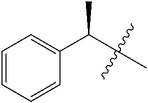 | T129a |
| 1459 | 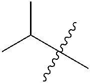 | 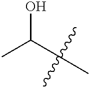 | 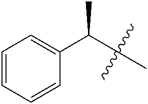 | T59b |
| 1460 | 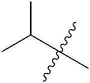 | 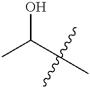 | 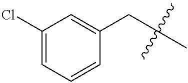 | T69 |
| 1461 | 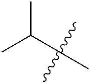 | 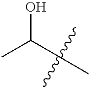 | 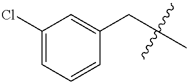 | T129a |
| 1462 | 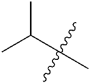 | 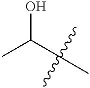 | 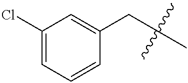 | T59b |
| 1463 | 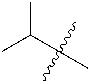 | 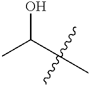 | 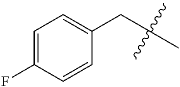 | T69 |
| 1464 | 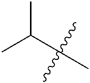 | 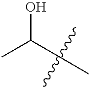 | 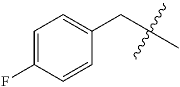 | T129a |
| 1465 | 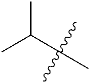 | 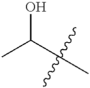 | 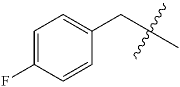 | T59b |
| 1466 | 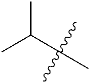 | 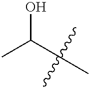 | 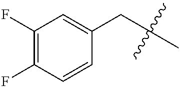 | T69 |
| 1467 | 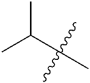 | 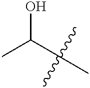 | 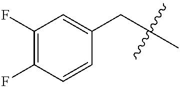 | T129a |
| 1468 | 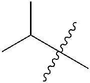 | 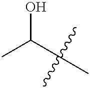 | 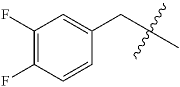 | T59b |
| 1469 | 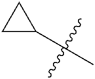 | 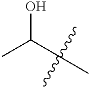 | 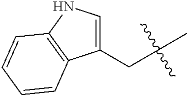 | T69 |
| 1470 | 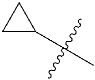 | 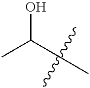 | 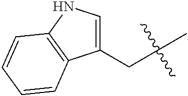 | T129a |
| 1471 | 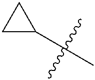 | 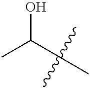 | 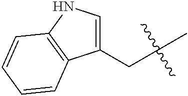 | T59b |
| 1472 | 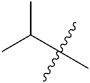 | 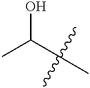 | 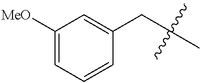 | T69 |
| 1473 | 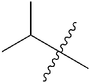 | 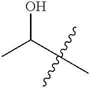 | 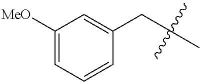 | T129a |
| 1474 | 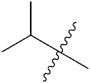 | 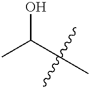 | 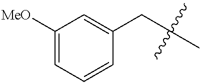 | T59b |
| 1475 | 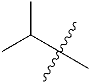 | 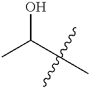 | 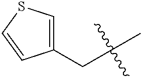 | T69 |
| 1476 | 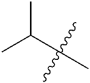 | 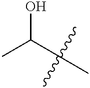 | 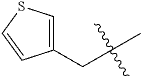 | T129a |
| 1477 | 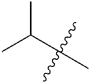 | 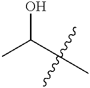 | 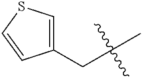 | T59b |
| 1478 | 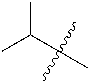 | 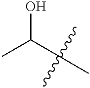 | 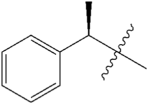 | T163a |
| 1479 | 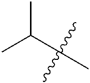 | 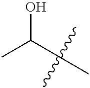 | 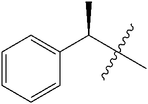 | T125a |
| 1480 | 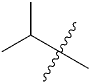 | 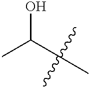 | 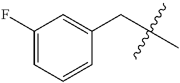 | T161a |
| 1481 | 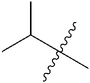 | 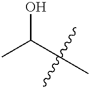 | 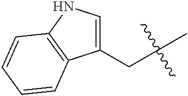 | T161a |
| 1482 | 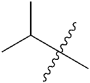 | 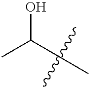 | 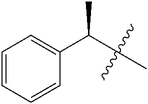 | T161a |
| 1483 | 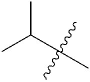 | 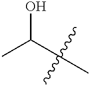 | 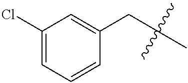 | T161a |
| 1484 | 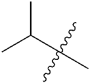 | 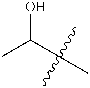 | 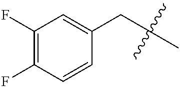 | T161a |
| 1485 | 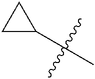 | 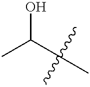 | 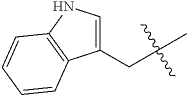 | T161a |
| 1486 | 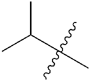 | 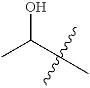 | 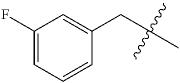 | T135 |
| 1487 | 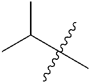 | 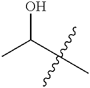 | 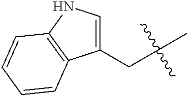 | T135 |
| 1488 | 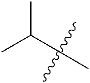 | 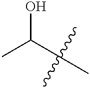 | 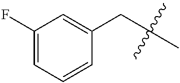 | T136 |
| 1489 | 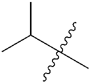 | 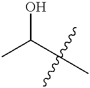 | 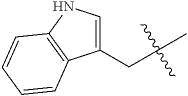 | T136 |
| 1490 | 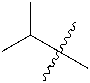 | 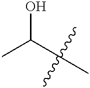 | 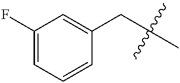 | T137 |
| 1491 | 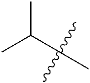 | 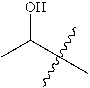 | 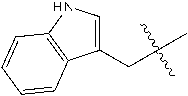 | T137 |
| 1492 | 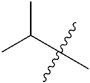 | 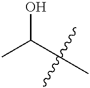 | 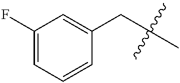 | T138 |
| 1493 | 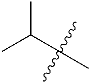 | 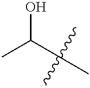 | 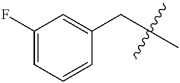 | T139 |
| 1494 | 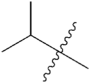 | 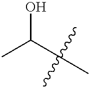 | 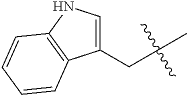 | T138 |
| 1495 | 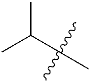 | 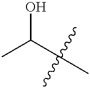 | 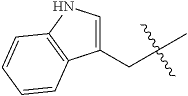 | T139 |
| 1496 | 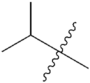 | 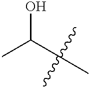 | 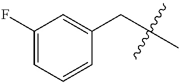 | T140a |
| 1497 | 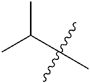 | 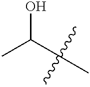 | 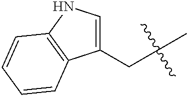 | T140a |
| 1498 | 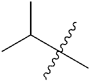 | 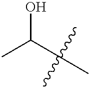 | 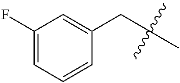 | T143 |
| 1499 | 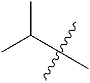 | 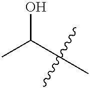 | 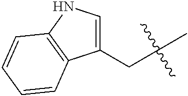 | T143 |
| 1500 | 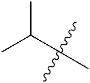 | 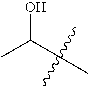 | 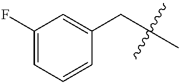 | T144b |
| 1501 | 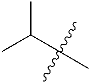 | 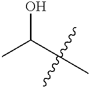 | 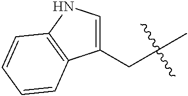 | T127a |
| 1502 | 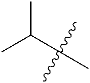 | 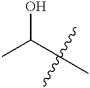 | 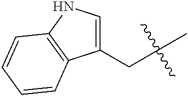 | T144b |
| 1503 | 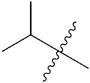 | 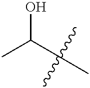 | 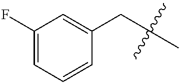 | T148c |
| 1504 | 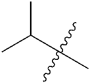 | 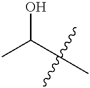 | 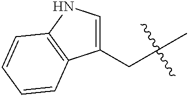 | T148c |
| 1505 | 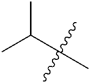 | 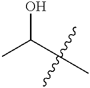 | 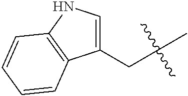 | T134a |
| 1506 | 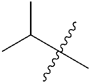 | 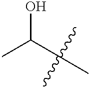 | 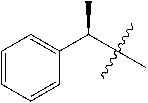 | T134a |
| 1507 | 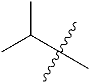 | 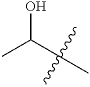 | 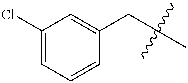 | T134a |
| 1508 | 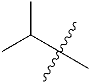 | 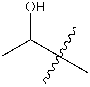 | 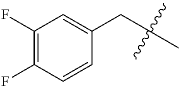 | T134a |
| 1509 | 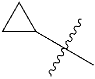 | 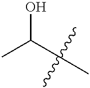 | 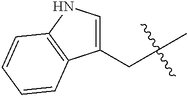 | T134a |
| 1510 | 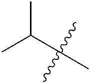 | 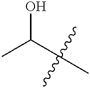 | 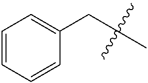 | T134a |
| 1511 | 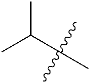 | 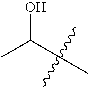 | 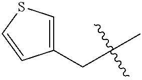 | T134a |
| 1512 | 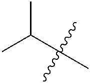 | 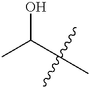 | 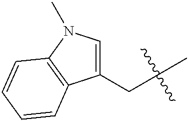 | T125a |
| 1513 | 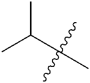 | 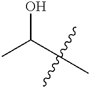 | 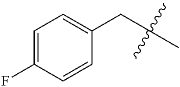 | T161a |
| 1514 | 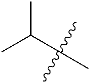 | 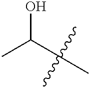 | 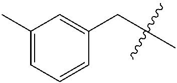 | T161a |
| 1515 | 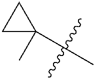 | 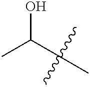 | 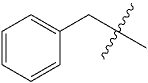 | T125a |
| 1516 | 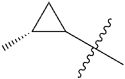 | 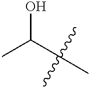 | 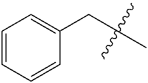 | T125a |
| 1517 | 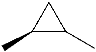 | 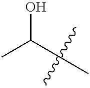 | 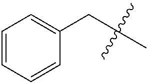 | T125a |
| 1518 | 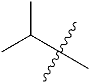 | 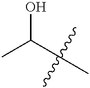 | 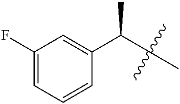 | T125a |
| 1519 | 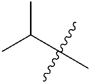 | 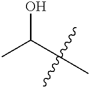 | 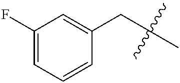 | T77 |
| 1520 | 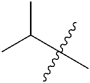 | 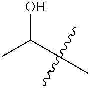 | 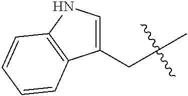 | T77 |
| 1521 | 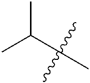 | 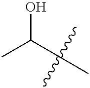 | 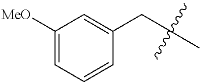 | T161a |
| 1522 | 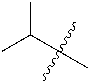 | 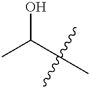 | 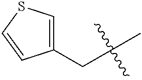 | T161a |
| 1523 | 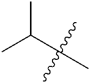 | 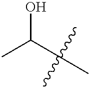 | 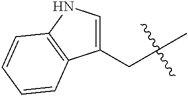 | T146b |
| 1524 | 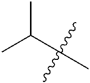 | 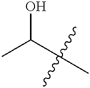 | 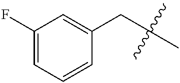 | T147 |
| 1525 | 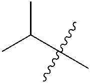 | 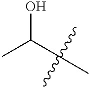 | 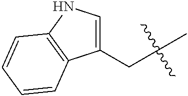 | T147 |
| 1526 | 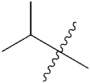 | 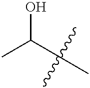 | 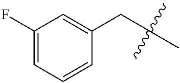 | T127a |
| 1527 | 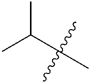 | 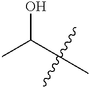 | 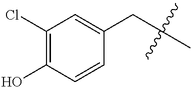 | T161a |
| 1528 | 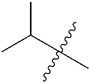 | 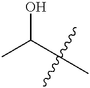 | 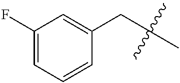 | T134a |
| 1529 | 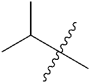 | 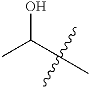 | 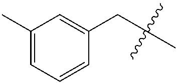 | T134a |
| 1530 | 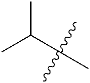 | 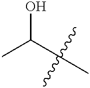 | 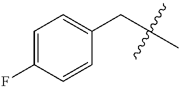 | T134a |
| 1531 | 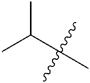 | 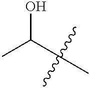 | 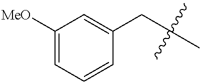 | T134a |
| 1532 | 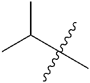 | 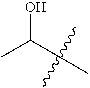 | 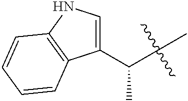 | T125a |
| 1533 | 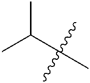 | 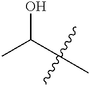 | 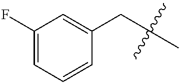 | T141 |
| 1534 | 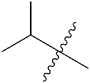 | 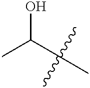 | 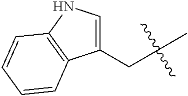 | T141 |
| 1535 | 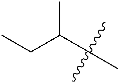 | 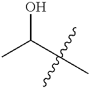 | 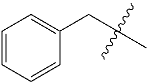 | T154 |
| 1551 | 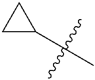 | 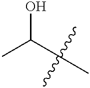 | 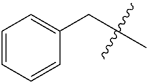 | T165a |
| 1552 | 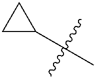 | 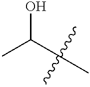 | 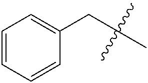 | T165b |
| 1553 | 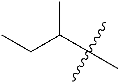 | 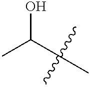 | 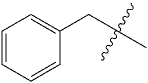 | T105 |
| 1554 | 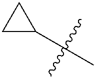 | 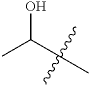 | 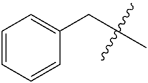 | T105 |
| 1555 | 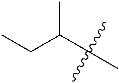 | 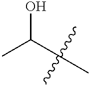 | 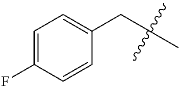 | T66 |
| 1556 | 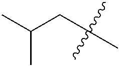 | 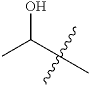 | 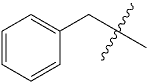 | T8 |
| 1558 | 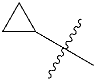 | 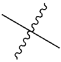 | 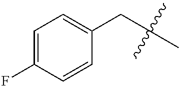 | T105 |
| 1559 | 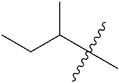 | 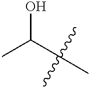 | 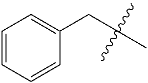 | T106 |
| 1560 | 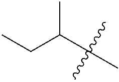 | 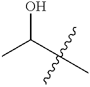 | 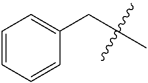 | T113b |
| 1565 | 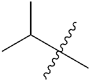 | 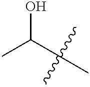 | 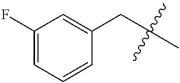 | T142 |
| 1566 | 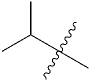 | 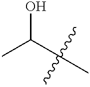 | 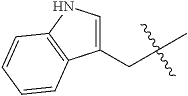 | T142 |
| 1601 | 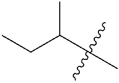 | 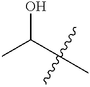 | 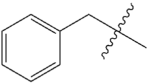 | T104 |
| 1602 | 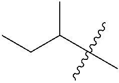 | 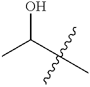 | 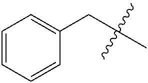 | T104a |
| 1603 | 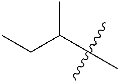 | 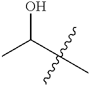 | 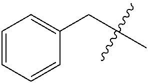 | T104b |
| 1604 | 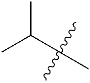 | 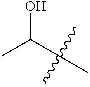 | 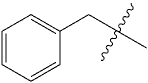 | T104b |
| 1605 | 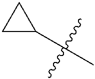 | 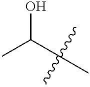 | 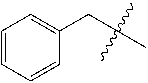 | T104b |
| 1606 | 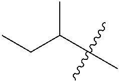 | 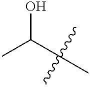 | 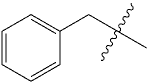 | T168b |
| 1607 | 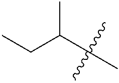 | 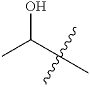 | 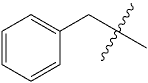 | T168b |
| 1608 | 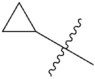 | 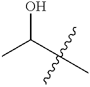 | 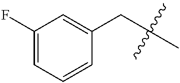 | T168b |
| 1609 | 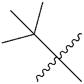 | 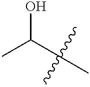 | 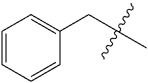 | T168b |
| 1610 | 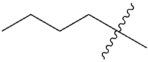 | 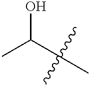 | 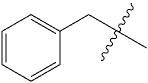 | T168b |
| 1611 | 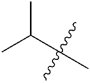 | 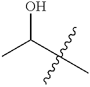 | 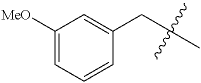 | T168b |
| 1612 | 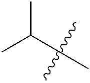 | 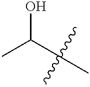 | 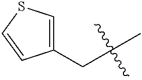 | T168b |
| 1613 | 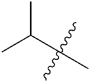 | 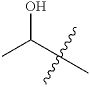 | 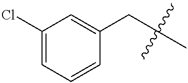 | T168b |
| 1614 | 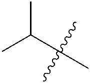 | 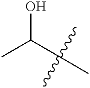 | 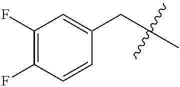 | T168b |
| 1615 | 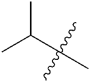 | 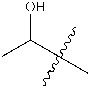 | 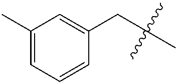 | T168b |
| 1616 | 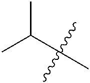 | 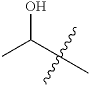 | 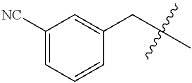 | T168b |
| 1617 | 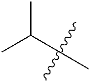 | 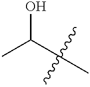 | 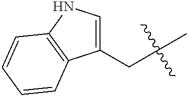 | T168b |
| 1618 | 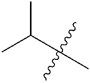 | 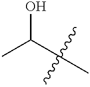 | 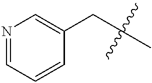 | T168b |
| 1619 | 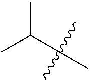 | 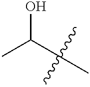 | 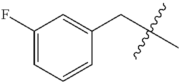 | T104b |
| 1620 | 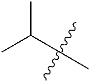 | 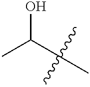 | 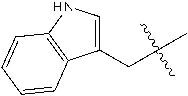 | T104b |
| 1621 | 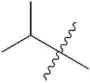 | 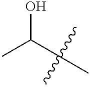 | 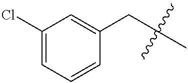 | T104b |
| 1622 | 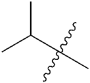 | 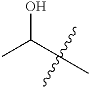 | 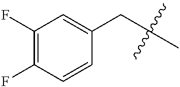 | T104b |
| 1623 | 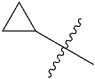 | 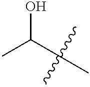 | 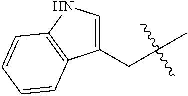 | T104b |
| 1624 | 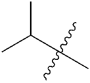 | 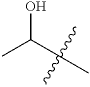 | 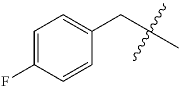 | T104b |
| 1625 | 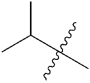 | 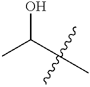 | 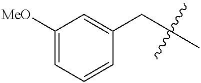 | T104b |
| 1626 | 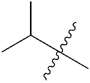 | 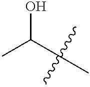 | 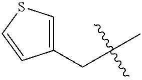 | T104b |
| 1627 | 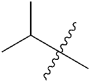 | 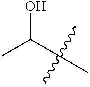 | 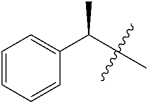 | T104b |
| 1628 | 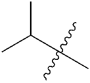 | 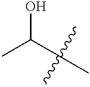 | 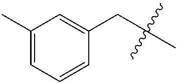 | T104b |
| 1629 | 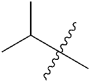 | 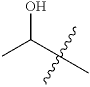 | 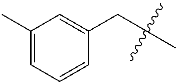 | T104b |
| 1630 | 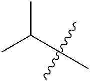 | 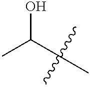 | 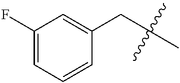 | T149b |
| 1631 | 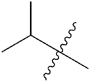 | 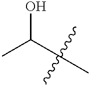 | 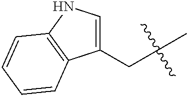 | T149b |
| 1632 | 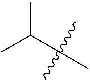 | 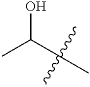 | 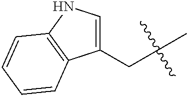 | T150b |
| 1633 | 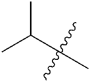 | 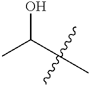 | 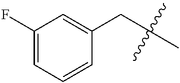 | T150b |
| 1634 | 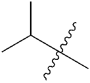 | 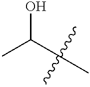 | 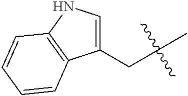 | T150a |
| 1635 | 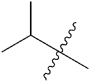 | 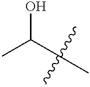 | 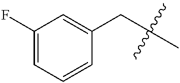 | T150a |
| 1636 | 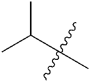 | 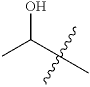 | 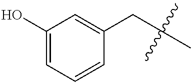 | T104 |
| 1655 | 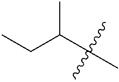 | 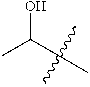 | 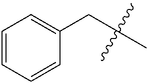 | T153 |
| 1688 | 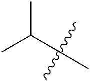 | 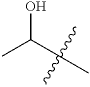 | 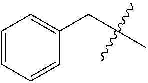 | T127a |
| 1689 | 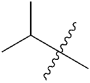 | 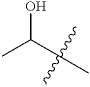 | 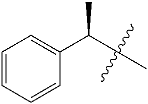 | T135 |
| 1690 | 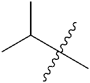 | 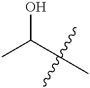 | 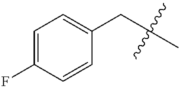 | T135 |
| 1691 | 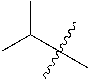 | 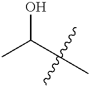 | 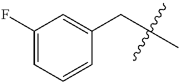 | T65 |
| 1692 | 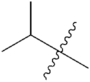 | 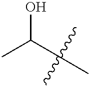 | 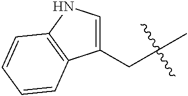 | T65 |
| 1693 | 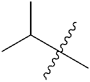 | 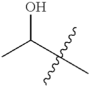 | 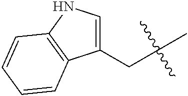 | T187 |
| 1694 | 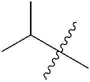 | 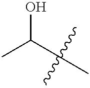 | 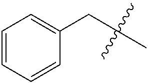 | T172a |
| 1695 | 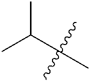 | 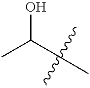 | 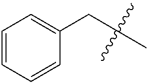 | T173a |
| 1696 | 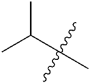 | 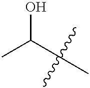 | 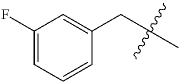 | T172a |
| 1697 | 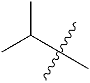 | 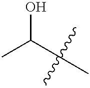 | 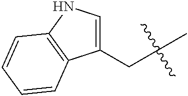 | T172a |
| 1698 | 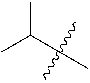 | 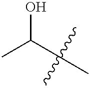 | 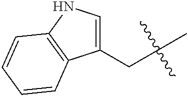 | T173a |
| 1699 | 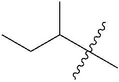 | 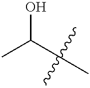 | 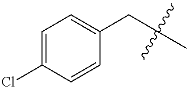 | T9 |
| 1700 | 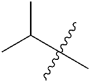 | 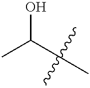 | 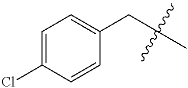 | T127a |
| 1701 | 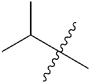 | 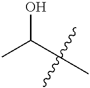 | 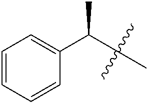 | T127a |
| 1702 | 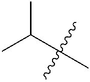 | 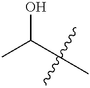 | 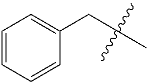 | T135 |
| 1703 | 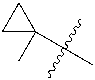 | 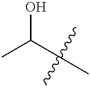 | 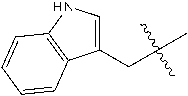 | T134a |
| 1704 | 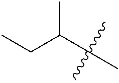 | 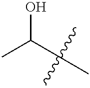 | 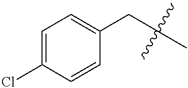 | T65 |
| 1705 | 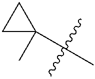 | 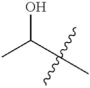 | 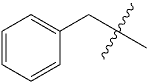 | T181a |
| 1706 | 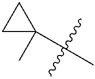 | 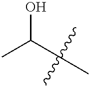 | 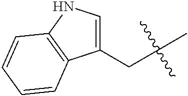 | T181a |
| 1707 | 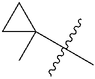 | 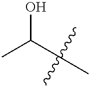 | 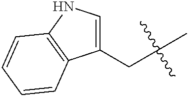 | T180a |
| 1708 | 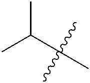 | 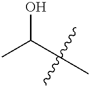 | 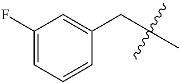 | T173a |
| 1709 | 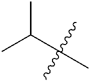 | 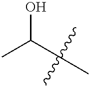 | 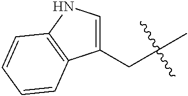 | T188a |
| 1710 | 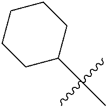 | 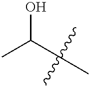 | 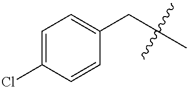 | T8 |
| 1711 | 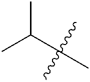 | 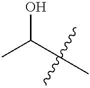 | 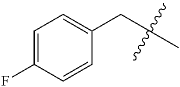 | T127a |
| 1712 | 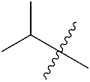 | 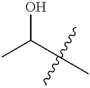 | 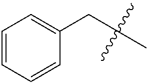 | T65 |
| 1713 | 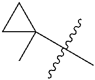 | 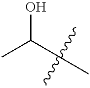 | 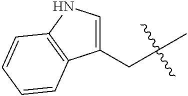 | T127a |
| 1714 | 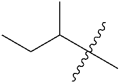 | 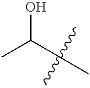 | 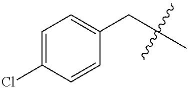 | T149b |
| 1715 | 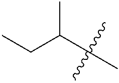 | 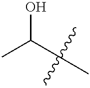 | 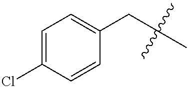 | T104b |
| 1718 | 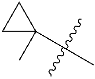 | 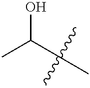 | 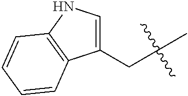 | T182a |
| 1719 | 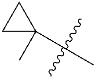 | 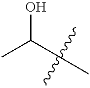 | 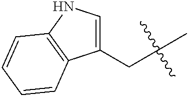 | T179a |
| 1720 | 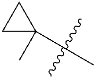 | 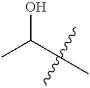 | 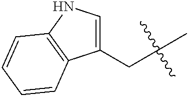 | T178a |
| 1721 | 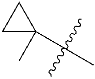 | 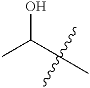 | 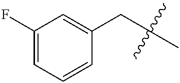 | T181a |
| 1722 | 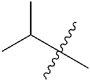 | 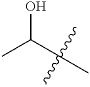 | 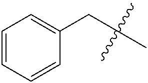 | T185a |
| 1723 | 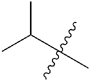 | 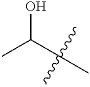 | 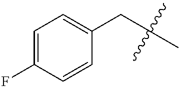 | T185a |
| 1724 | 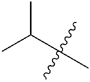 | 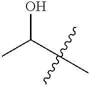 | 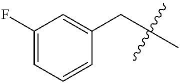 | T185a |
| 1725 | 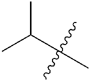 | 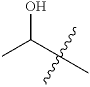 | 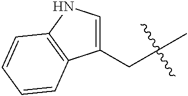 | T185a |
| 1726 | 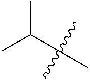 | 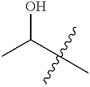 | 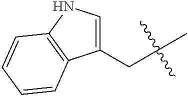 | T184a |
| 1727 | 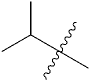 | 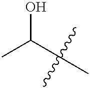 | 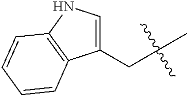 | T171a |
| 1728 | 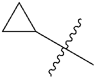 | 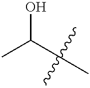 | 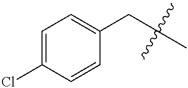 | T8 |
| 1729 | 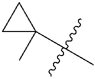 | 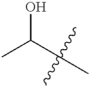 | 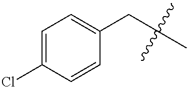 | T8 |
| 1730 | 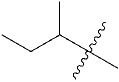 | 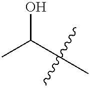 | 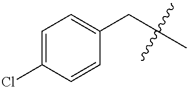 | T8 |
| 1731 | 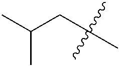 | 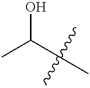 | 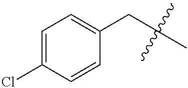 | T8 |
| 1732 | 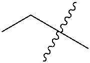 | 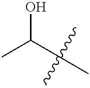 | 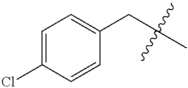 | T8 |
| 1733 | 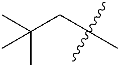 | 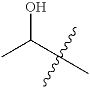 | 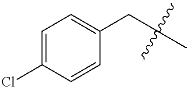 | T8 |
| 1735 | 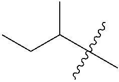 | 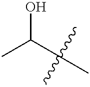 | 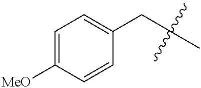 | T8 |
| 1736 | 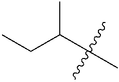 | 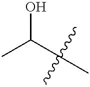 | 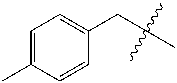 | T8 |
| 1737 | 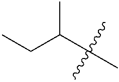 | 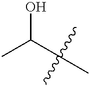 | 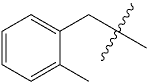 | T8 |
| 1738 | 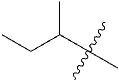 | 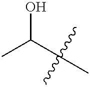 | 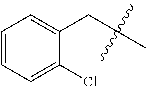 | T8 |
| 1739 | 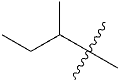 | 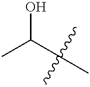 | 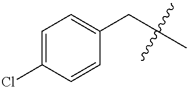 | T135 |
| 1740 | 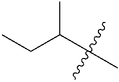 | 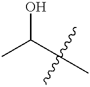 | 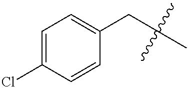 | T136 |
| 1741 | 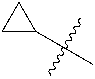 | 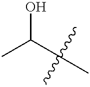 | 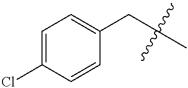 | T128b |
| 1742 | 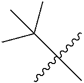 | 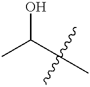 | 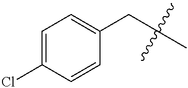 | T125a |
| 1743 | 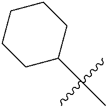 | 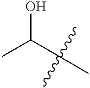 | 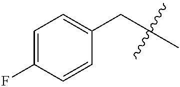 | T125a |
| 1744 | 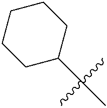 | 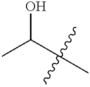 | 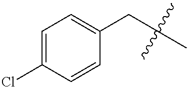 | T125a |
| 1745 | 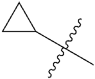 | 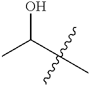 | 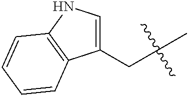 | T125a |
| 1746 | 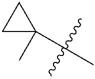 | 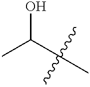 | 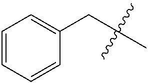 | T134a |
| 1747 | 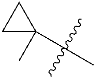 | 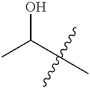 | 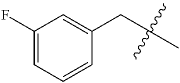 | T134a |
| 1751 | 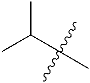 | 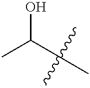 | 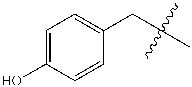 | T134a |
| 1752 | 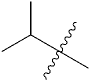 | 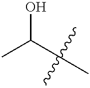 | 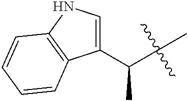 | T134a |
| 1753 | 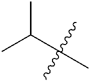 | 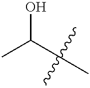 | 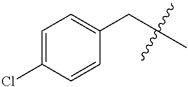 | T134a |
| 1754 | 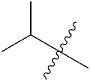 | 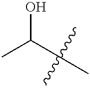 | 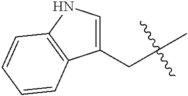 | T177a |
| 1755 | 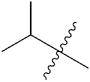 | 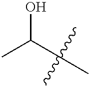 | 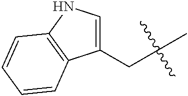 | T186a |
| 1756 | 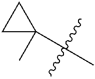 | 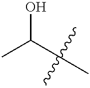 | 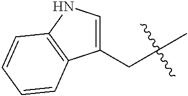 | T183a |
| 1757 | 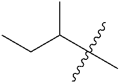 | 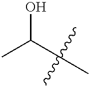 | 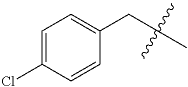 | T154 |
| 1758 | 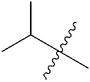 | 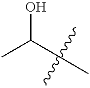 | 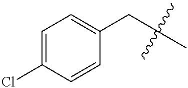 | T129a |
| 1759 | 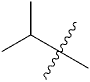 | 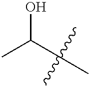 | 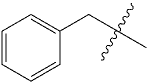 | T186a |
| 1760 | 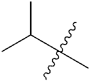 | 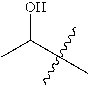 | 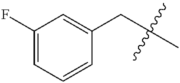 | T186a |
| 1761 | 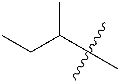 | 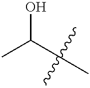 | 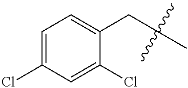 | T8 |
| 1762 | 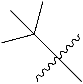 | 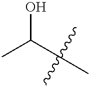 | 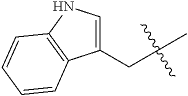 | T125a |
| 1763 | 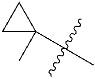 | 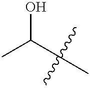 | 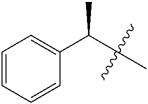 | T134a |
| 1764 | 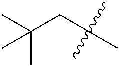 | 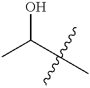 | 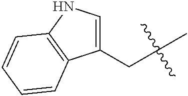 | T134a |
| 1768 | 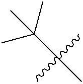 | 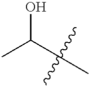 | 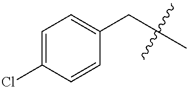 | T8 |
| 1769 | 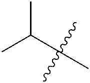 | 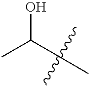 | 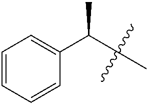 | T137 |
| 1770 | 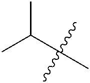 | 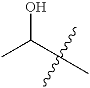 | 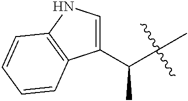 | T137 |
| 1771 | 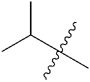 | 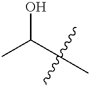 | 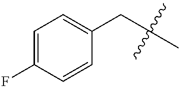 | T137 |
| 1772 | 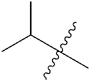 | 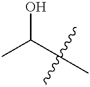 | 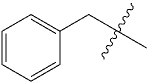 | T137 |
| 1773 | 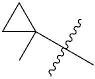 | 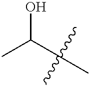 | 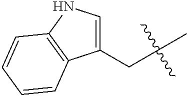 | T137 |
| 1774 | 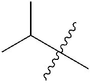 | 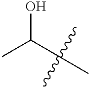 | 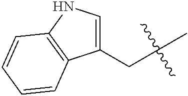 | T175 |
| 1775 | 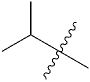 | 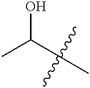 | 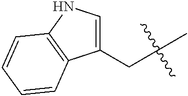 | T176 |
| 1776 | 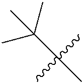 | 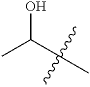 | 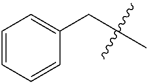 | T153 |
| 1777 | 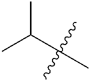 | 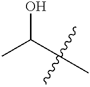 | 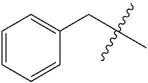 | T153 |
| 1778 | 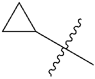 | 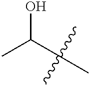 | 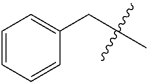 | T153 |
| 1779 | 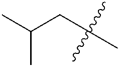 | 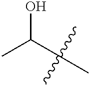 | 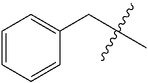 | T153 |
| 1780 | 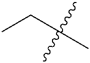 | 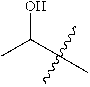 | 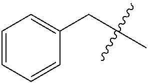 | T153 |
| 1781 | 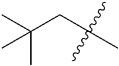 | 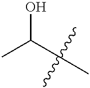 | 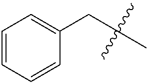 | T153 |
| 1782 | 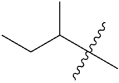 | 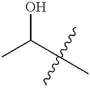 | 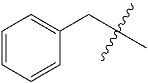 | T153 |
| 1784 | 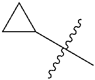 | 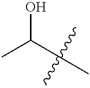 | 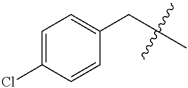 | T125b |
| 1785 | 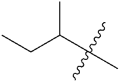 | 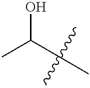 | 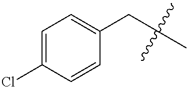 | T125b |
| 1786 | 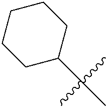 | 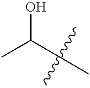 | 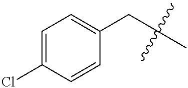 | T125b |
| 1787 | 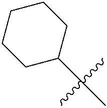 | 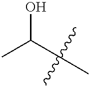 | 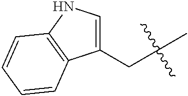 | T125b |
| 1789 | 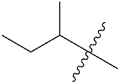 | 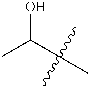 | 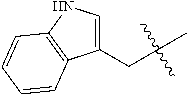 | T153 |
| 1790 | 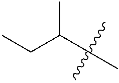 | 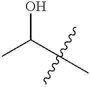 | 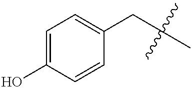 | T153 |
| 1791 | 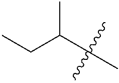 | 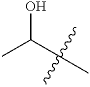 | 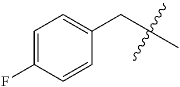 | T153 |
| 1792 | 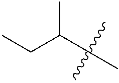 | 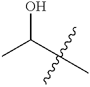 | 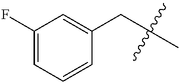 | T153 |
| 1794 | 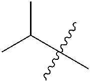 | 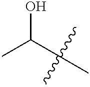 | 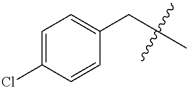 | T8 |
| 1795 | 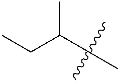 | 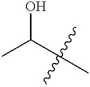 | 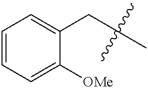 | T8 |
| 1796 | 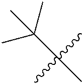 | 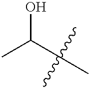 | 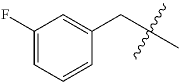 | T125a |
| 1797 | 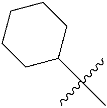 | 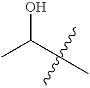 | 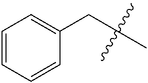 | T153 |
| 1798 | 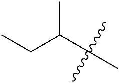 | 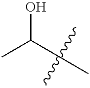 | 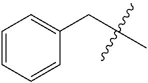 | T153 |
| 1799 | 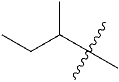 | 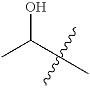 | 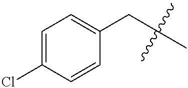 | T137 |
| 1800 | 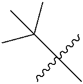 | 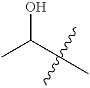 | 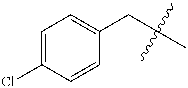 | T125b |
| 1801 | 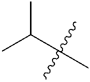 | 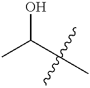 | 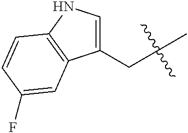 | T125a |
| 1802 | 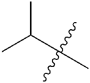 | 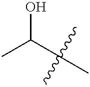 | 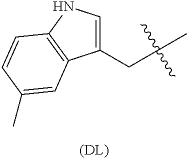 | T125a |
| 1803 | 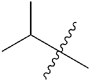 | 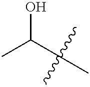 | 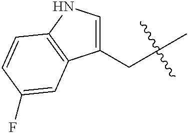 | T134a |
| 1805 | 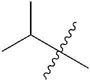 | 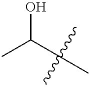 | 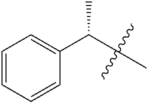 | T134a |
| 1806 | 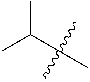 | 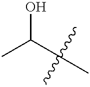 | 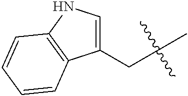 | T189a |
| 1808 | 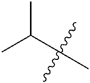 | 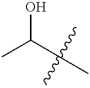 | 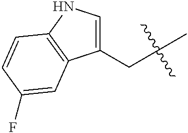 | T161a |
| 1809 | 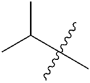 | 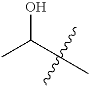 | 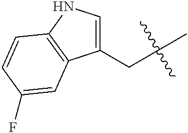 | T127a |
| 1810 | 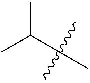 | 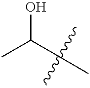 | 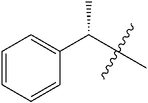 | T127a |
| 1811 | 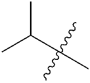 | 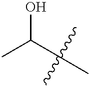 | 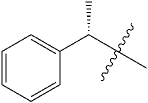 | T137 |
| 1812 | 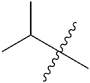 | 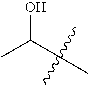 | 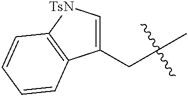 | T134a |
| 1813 | 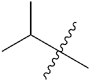 | 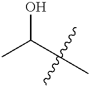 | 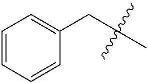 | T189a |
| 1814 | 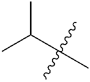 | 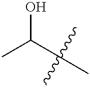 | 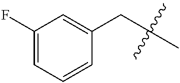 | T189a |
| 1815 | 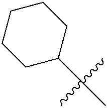 | 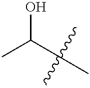 | 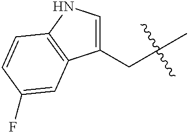 | T125a |
| 1824 | 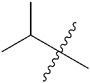 | 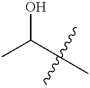 | 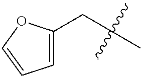 | T134a |
| 1825 | 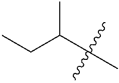 | 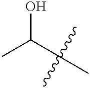 | 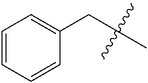 | T153a |
| 1826 | 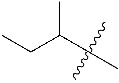 | 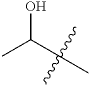 | 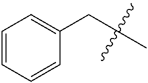 | T153b |
| 1827 | 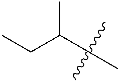 | 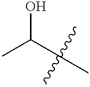 | 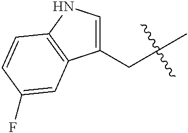 | T8 |
| 1829 | 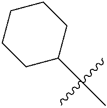 | 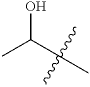 | 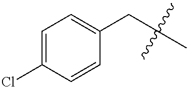 | T8 |
| 1830 | 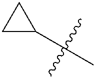 | 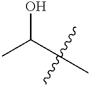 | 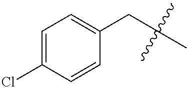 | T8 |
| 1831 | 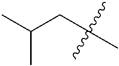 | 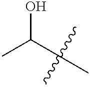 | 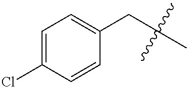 | T8 |
| 1832 | 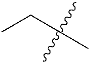 | 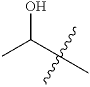 | 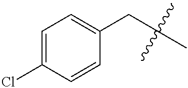 | T8 |
| 1834 | 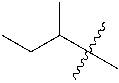 | 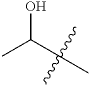 | 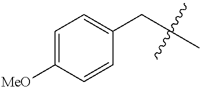 | T8 |
| 1835 | 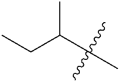 | 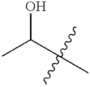 | 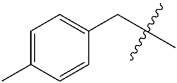 | T8 |
| 1836 | 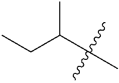 | 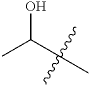 | 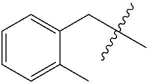 | T8 |
| 1837 | 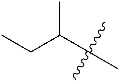 | 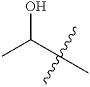 | 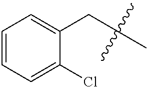 | T8 |
| 1838 | 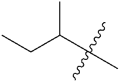 | 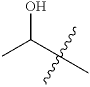 | 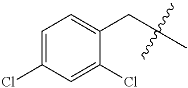 | T8 |
| 1839 | 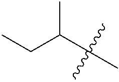 | 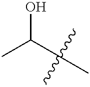 | 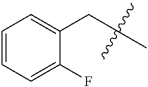 | T153 |
| 1840 | 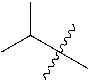 | 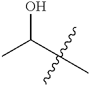 | 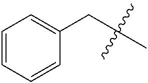 | T222 |
| 1841 | 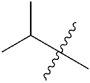 | 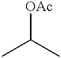 | 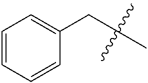 | T8 |
| 1842 | 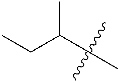 | 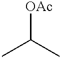 | 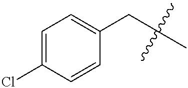 | T8 |
| 1843 | 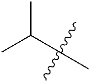 | 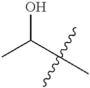 | 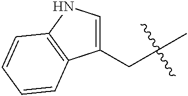 | T193 |
| 1844 | 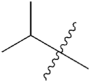 | 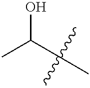 | 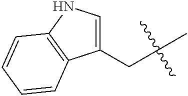 | T193 |
| 1846 | 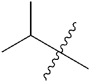 | 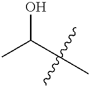 | 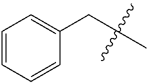 | T210a |
| 1847 | 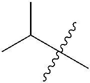 | 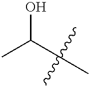 | 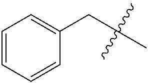 | T211a |
| 1848 | 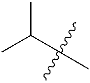 | 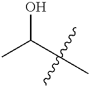 | 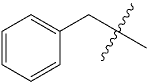 | T193 |
| 1849 | 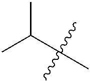 | 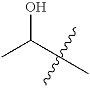 | 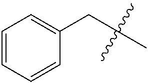 | T193 |
| 1851 | 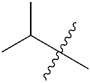 | 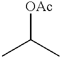 | 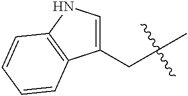 | T134a |
| 1852 | 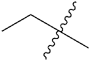 | 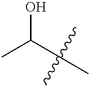 | 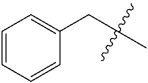 | T181a |
| 1853 | 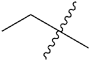 | 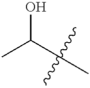 | 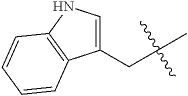 | T134a |
| 1854 | 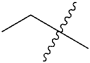 | 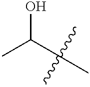 | 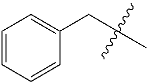 | T134a |
| 1855 | 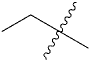 | 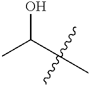 | 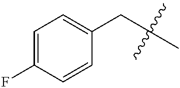 | T134a |
| 1856 | 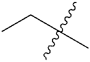 | 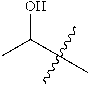 | 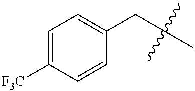 | T134a |
| 1857 | 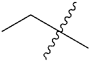 | 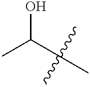 | 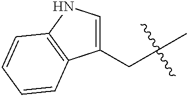 | T181a |
| 1858 | 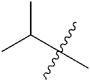 | 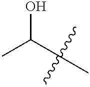 | 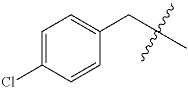 | T153 |
| 1859 | 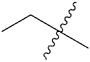 | 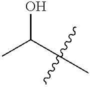 | 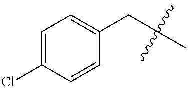 | T153 |
| 1860 | 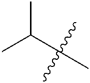 | 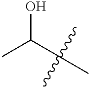 | 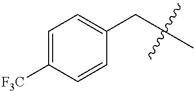 | T153 |
| 1861 | 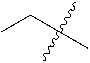 | 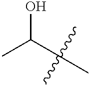 | 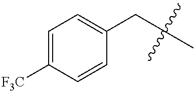 | T153 |
| 1862 | 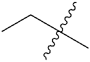 | 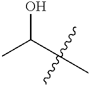 | 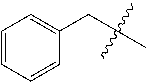 | T179a |
| 1863 | 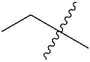 | 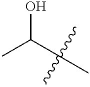 | 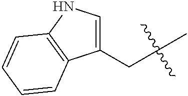 | T179a |
| 1864 | 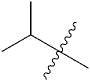 | 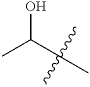 | 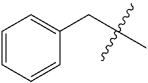 | T212a |
| 1866 | 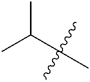 | 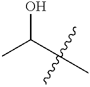 | 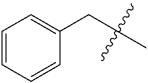 | T213a |
| 1867 | 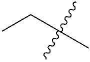 | 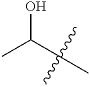 | 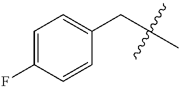 | T134a |
| 1869 | 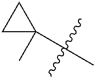 | 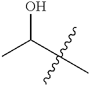 | 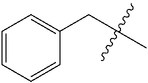 | T179a |
| 1870 | 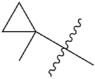 | 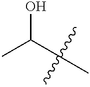 | 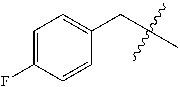 | T179a |
| 1871 | 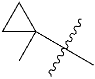 | 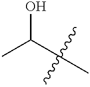 | 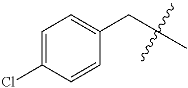 | T179a |
| 1872 | 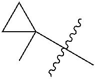 | 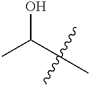 | 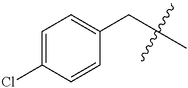 | T129a |
| 1875 | 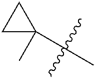 | 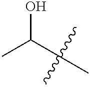 | 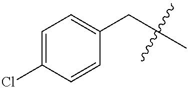 | T134a |
| 1876 | 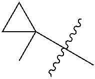 | 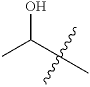 | 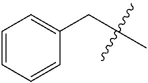 | T176 |
| 1878 | 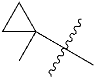 | 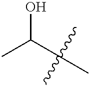 | 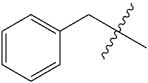 | T65 |
| 1879 | 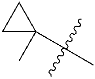 | 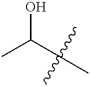 | 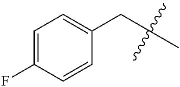 | T65 |
| 1880 | 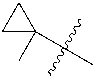 | 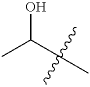 | 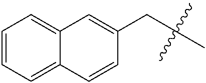 | T77 |
| 1881 | 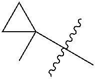 | 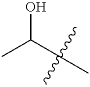 | 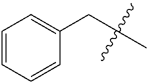 | T153 |
| 1882 | 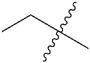 | 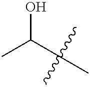 | 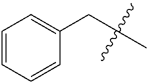 | T214a |
| 1883 | 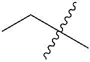 | 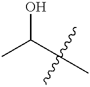 | 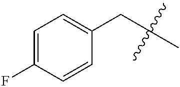 | T214a |
| 1884 | 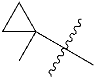 | 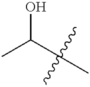 | 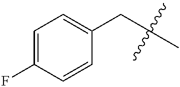 | T176 |
| 1885 | 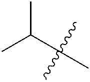 | 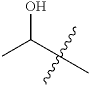 | 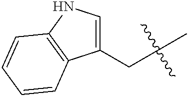 | T215 |
| 1888 | 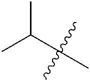 | 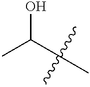 | 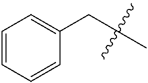 | T217a |
| 1889 | 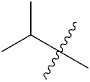 | 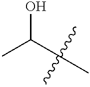 | 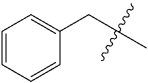 | T220a |
| 1890 | 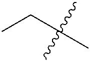 | 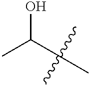 | 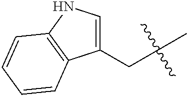 | T217a |
| 1891 | 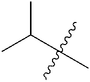 | 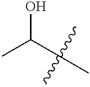 | 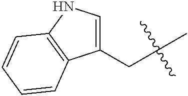 | T217a |
| 1892 | 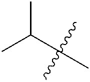 | 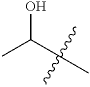 | 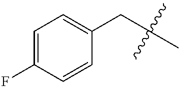 | T217a |
| 1893 | 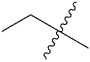 | 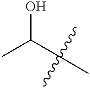 | 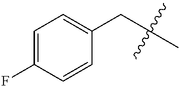 | T220a |
| 1894 | 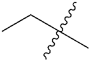 | 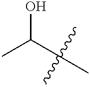 | 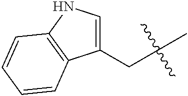 | T220a |
| 1895 | 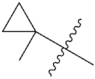 | 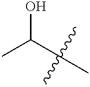 | 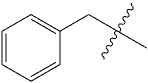 | T220a |
| 1896 | 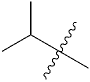 | 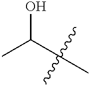 | 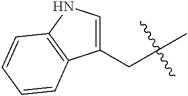 | T220a |
| 1897 | 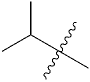 | 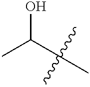 | 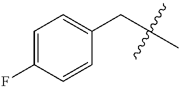 | T220a |
| 1898 | 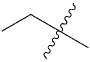 | 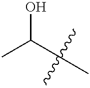 | 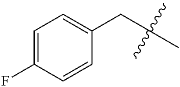 | T193 |
| 1899 | 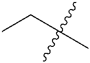 | 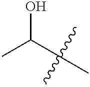 | 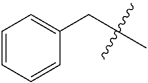 | T193 |
| 1900 | 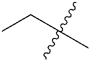 | 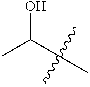 | 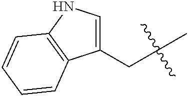 | T193 |
| 1901 | 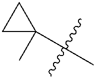 | 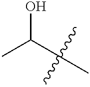 | 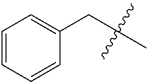 | T193 |
| 1902 | 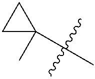 | 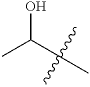 | 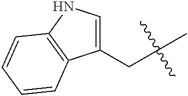 | T193 |
| 1903 | 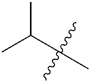 | 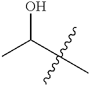 | 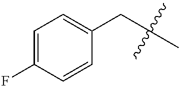 | T193 |
| 1904 | 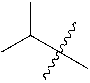 | 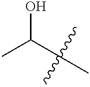 | 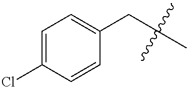 | T193 |
| 1905 | 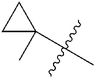 | 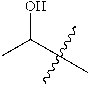 | 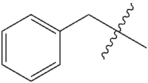 | T216a |
| 1906 | 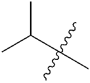 | 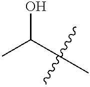 | 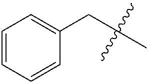 | T219a |
| 1907 | 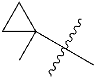 | 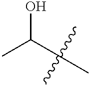 | 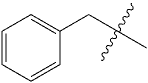 | T219a |
| 1909 | 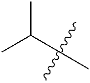 | 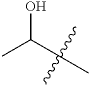 | 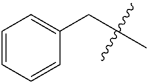 | T216a |
| 1911 | 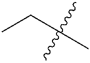 | 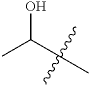 | 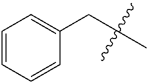 | T217a |
| 1912 | 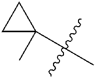 | 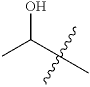 | 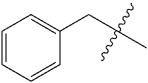 | T217a |
| 1913 | 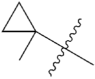 | 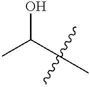 | 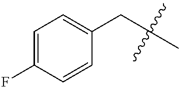 | T134a |
| 1914 | 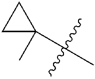 | 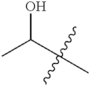 | 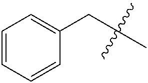 | T218a |
| 1916 | 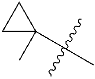 | 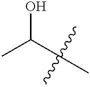 | 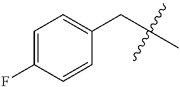 | T129a |
| 1918 | 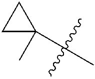 | 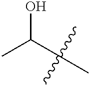 | 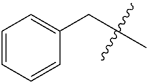 | T187 |
| 1919 | 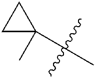 | 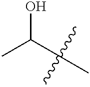 | 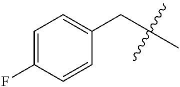 | T187 |
| 1921 | 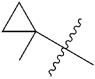 | 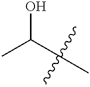 | 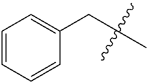 | T215 |
| 1922 | 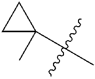 | 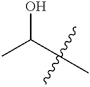 | 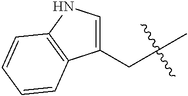 | T216a |
| 1925 | 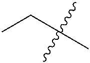 | 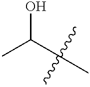 | 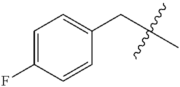 | T217a |
| 1927 | 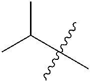 | 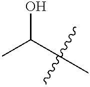 | 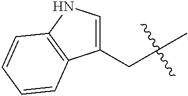 | T218a |
| 1928 | 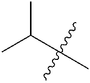 | 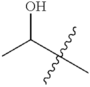 | 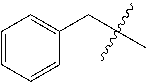 | T218a |
| 1929 | 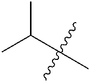 | 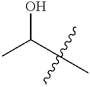 | 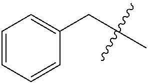 | T176 |
| 1930 | 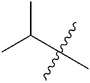 | 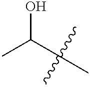 | 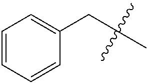 | T193 |

For the compounds in Table 1, Rₐ═H, R_{b}=Me, R_{c}═H, R_{d}═H for all compounds except the following: Rₐ=Me, compounds 1323, 1355, 1356; R_{b}═H, compounds 1353-1357, 1382; R_{c}=Me, compounds 1357, 1382, 1388, 1389; R_{d}=Me, compounds 1353, 1354.
The T_{A} elements of Table 1 are as follows:

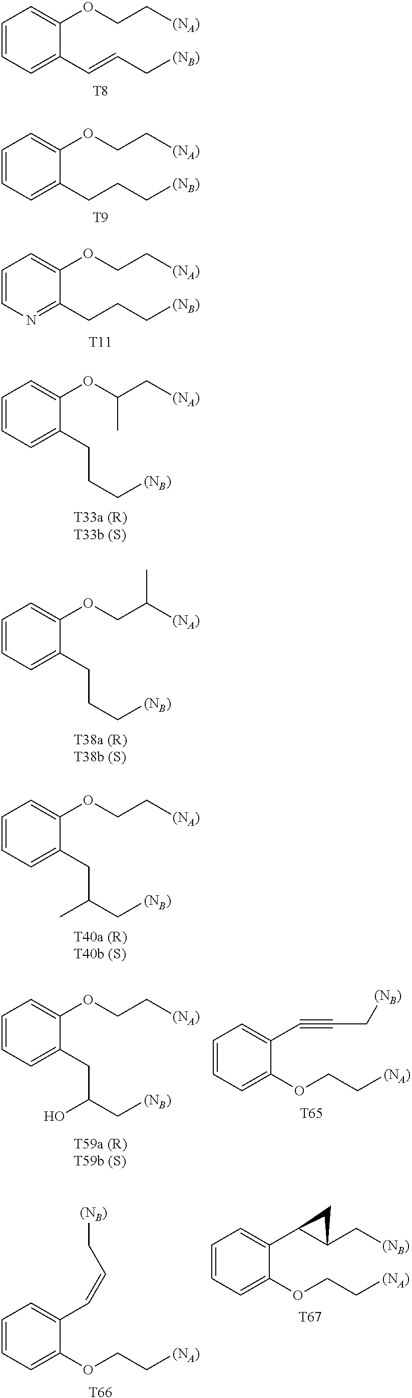

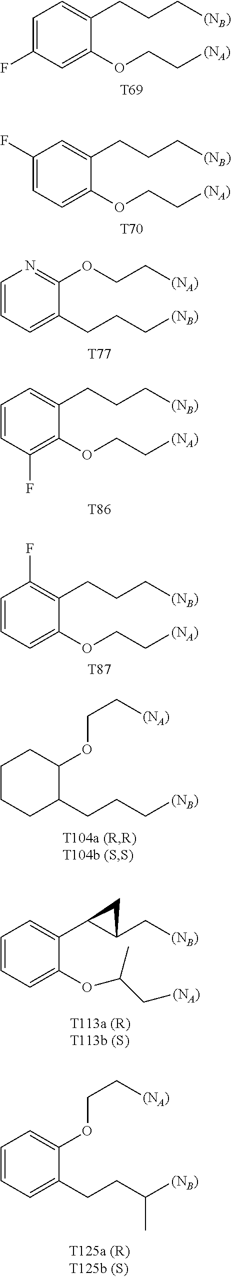

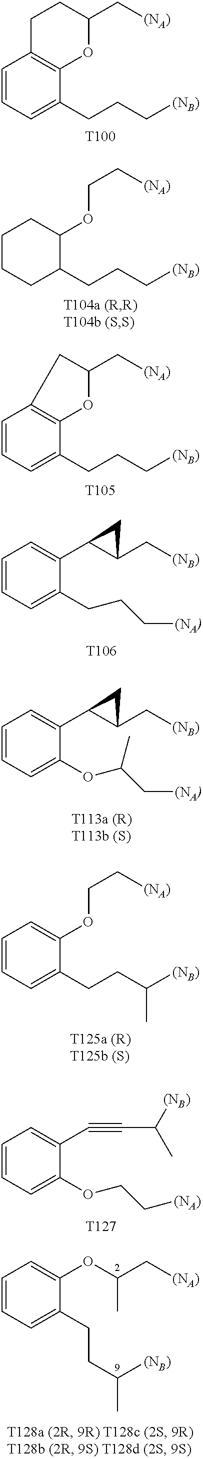

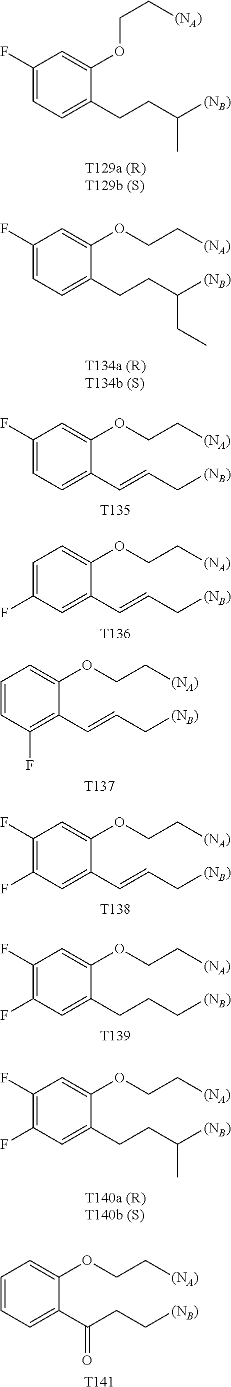

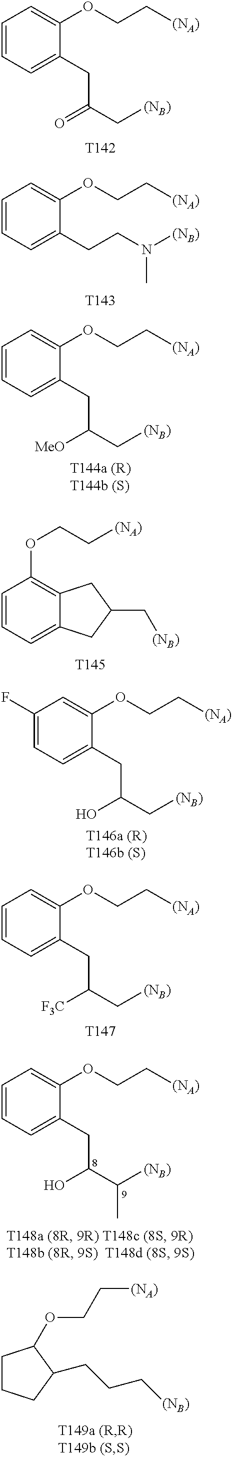

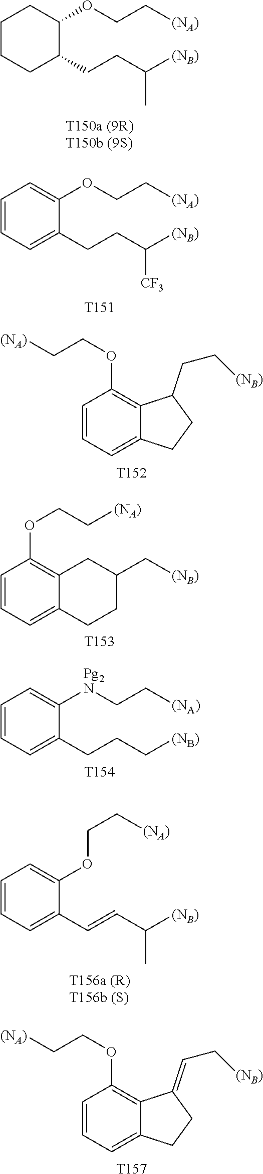

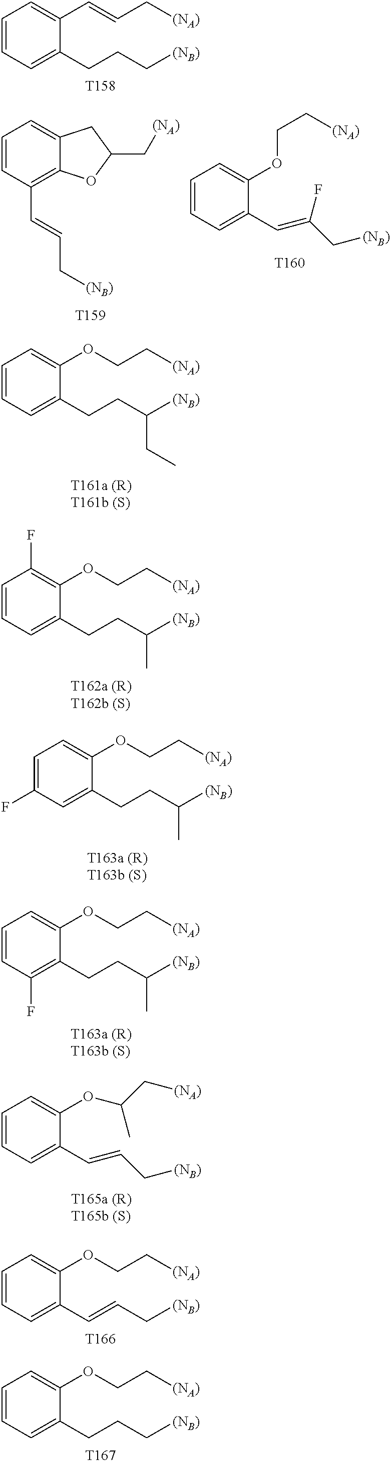

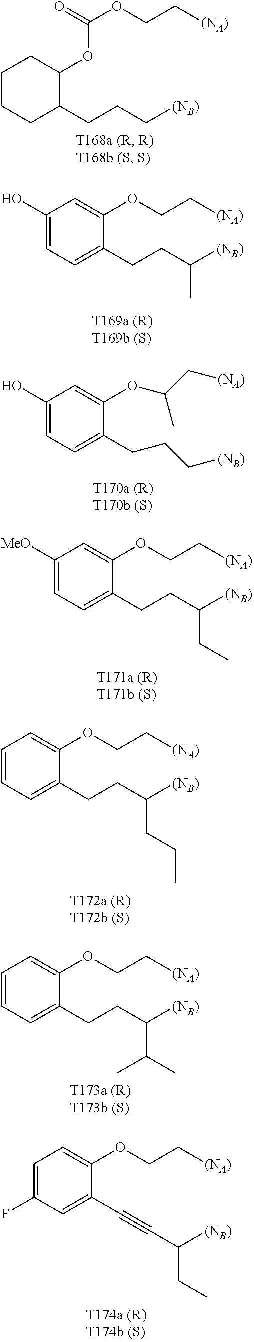

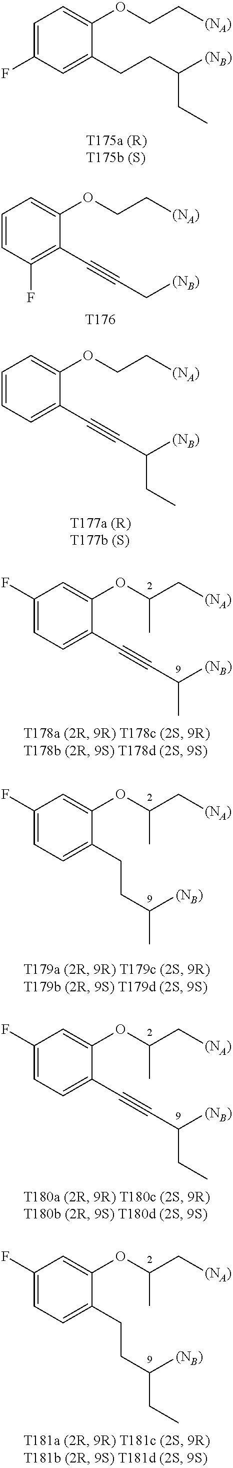

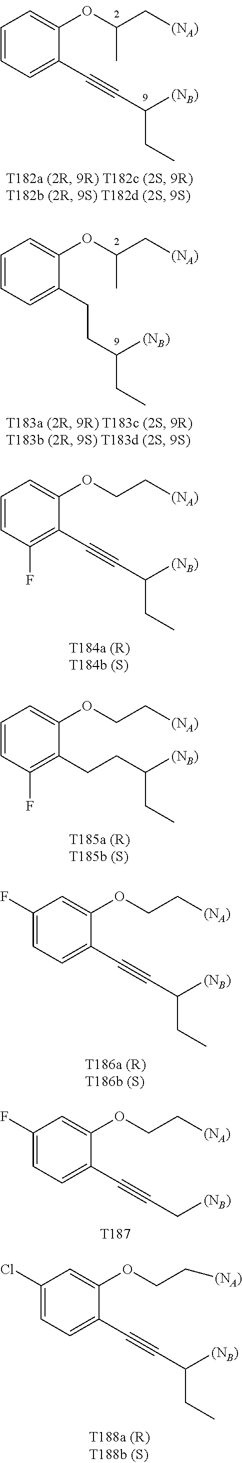

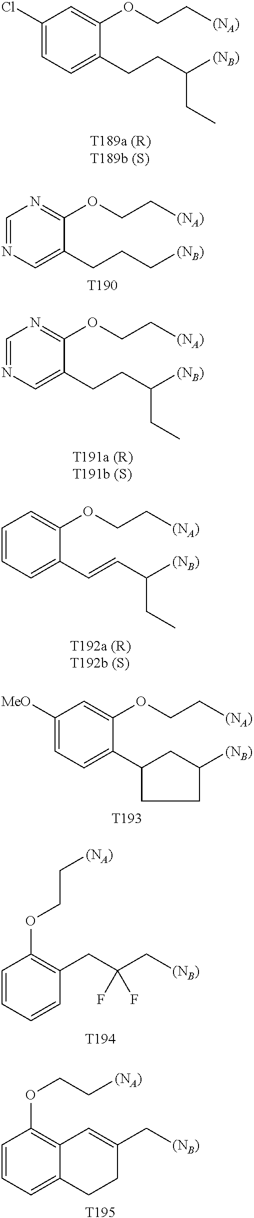

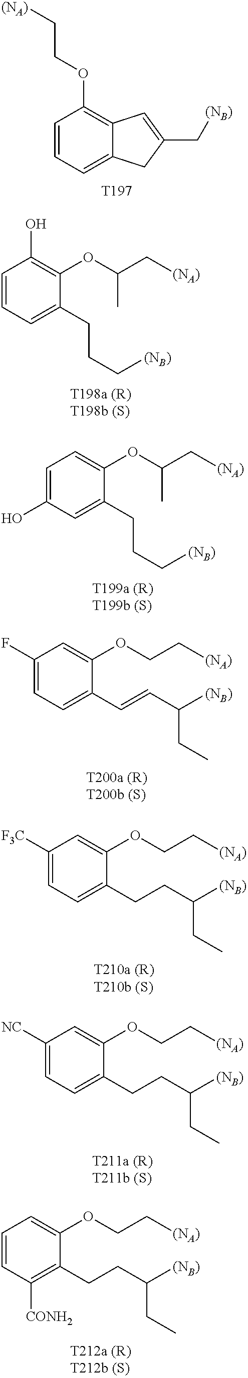

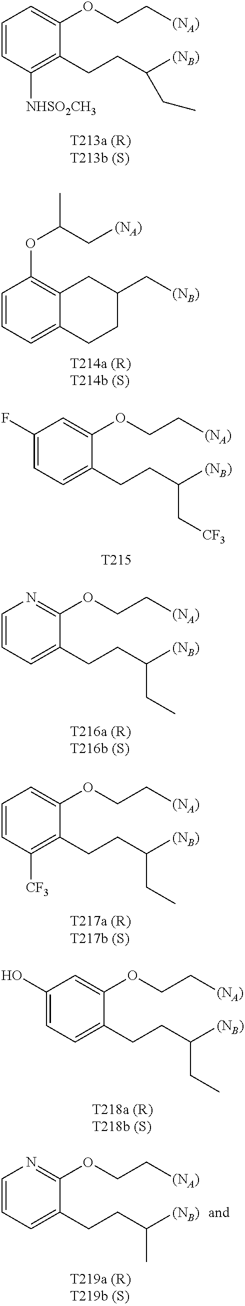

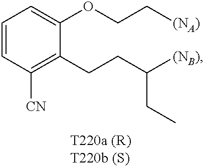

wherein (N_{A}) indicates the site of bonding to NRₐ of formula (A), (N_{B}) indicates the site of bonding to NR_{c} of formula (A) and Pg is a nitrogen protecting group.

The present invention includes isolated compounds. An isolated compound refers to a compound that, in some embodiments, comprises at least 10%, at least 25%, at least 50% or at least 70% of the compounds of a mixture. In some embodiments, the compound, pharmaceutically acceptable salt thereof or pharmaceutical composition containing the compound exhibits a statistically significant binding and/or antagonist activity and or inverse agonist activity when tested in biological assays at the human ghrelin receptor.

In the case of compounds, salts, or solvates that are solids, it is understood by those skilled in the art that the inventive compounds, salts, and solvates may exist in different crystal or polymorphic forms, all of which are intended to be within the scope of the present invention and specified formulas.

The compounds of formula (I) herein disclosed have asymmetric centers. The inventive compounds may exist as single stereoisomers, racemates, and/or mixtures of enantiomers and/or diastereomers. All such single stereoisomers, racemates, and mixtures thereof are intended to be within the scope of the present invention. However, the inventive compounds are used in optically pure form. The terms “S” and “R” configuration as used herein are as defined by the IUPAC 1974 Recommendations for Section E, Fundamentals of Stereochemistry (Pure Appl. Chem. 1976, 45, 13-30.).

Unless otherwise depicted to be a specific orientation, the present invention accounts for all stereoisomeric forms. The compounds may be prepared as a single stereoisomer or a mixture of stereoisomers. The non-racemic forms may be obtained by either synthesis or resolution. The compounds may, for example, be resolved into the component enantiomers by standard techniques, for example formation of diastereomeric pairs via salt formation. The compounds also may be resolved by covalently bonding to a chiral moiety. The diastereomers can then be resolved by chromatographic separation and/or crystallographic separation. In the case of a chiral auxiliary moiety, it can then be removed. As an alternative, the compounds can be resolved through the use of chiral chromatography. Enzymatic methods of resolution could also be used in certain cases.

As generally understood by those skilled in the art, an “optically pure” compound is one that contains only a single enantiomer. As used herein, the term “optically active” is intended to mean a compound comprising at least a sufficient excess of one enantiomer over the other such that the mixture rotates plane polarized light. The enantiomeric excess (e.e.) indicates the excess of one enantiomer over the other. Optically active compounds have the ability to rotate the plane of polarized light. In describing an optically active compound, the prefixes D and L or R and S are used to denote the absolute configuration of the molecule about its chiral center(s). The prefixes “d” and “l” or (+) and (−) are used to denote the optical rotation of the compound (i.e., the direction in which a plane of polarized light is rotated by the optically active compound). The “l” or (−) prefix indicates that the compound is levorotatory (i.e., rotates the plane of polarized light to the left or counterclockwise) while the “d” or (+) prefix means that the compound is dextrarotatory (i.e., rotates the plane of polarized light to the right or clockwise). The sign of optical rotation, (−) and (+), is not related to the absolute configuration of the molecule, R and S.

A compound of the invention having the desired pharmacological properties will be optically active and is comprised of at least 90% (80% e.e.), at least 95% (90% e.e.), at least 97.5% (95% e.e.) or at least 99% (98% e.e.) of a single isomer.

Likewise, many geometric isomers of double bonds and the like can also be present in the compounds disclosed herein, and all such stable isomers are included within the present invention unless otherwise specified. Also included in the invention are tautomers and rotamers of formula I.

The use of the following symbols at the right refers to substitution of one or more hydrogen atoms of the indicated ring with the defined substituent R.

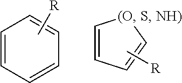

The use of the following symbol indicates a single bond or an optional double bond:

[symbol]

Embodiments of the present invention further provide intermediate compounds formed through the synthetic methods described herein to provide the compounds of formula (I). The intermediate may possess utility as a therapeutic agent and/or reagent for further synthesis methods and reactions.

### 2. Synthetic Methods

The compounds of formula (I) can be synthesized using traditional solution synthesis techniques or solid phase chemistry methods. In either, the construction involves four phases: first, synthesis of the building blocks comprising recognition elements for the biological target receptor, plus one tether moiety, primarily for control and definition of conformation. These building blocks are assembled together, typically in a sequential fashion, in a second phase employing standard chemical transformations. The precursors from the assembly are then cyclized in the third stage to provide the macrocyclic structures. Finally, the post-cyclization processing fourth stage involving removal of protecting groups and optional purification provides the desired final compounds. Synthetic methods for this general type of macrocyclic structure are described in Intl. Pat. Appls. WO 01/25257, WO 2004/111077, WO 2005/012331, WO 2005/012332, WO 2006/009645, WO 2006/009674, WO 2008/033328, WO 2008/130464 and U.S. Prov. Pat. Appl. 61/254,434 including purification procedures described in WO 2004/111077 and WO 2005/012331. Solution phase synthesis routes, including methods amenable to larger scale manufacture, were described in U.S. Patent Appl. Publ, Nos. 2006/025566 and US 2007/0021331.

In some embodiments of the present invention, the macrocyclic compounds of formula (I) may be synthesized using solid phase chemistry on a soluble or insoluble polymer matrix as previously defined. For solid phase chemistry, a preliminary stage involving the attachment of the first building block, also termed “loading,” to the resin must be performed. The resin utilized for the present invention preferentially has attached to it a linker moiety, L. These linkers are attached to an appropriate free chemical functionality, usually an alcohol or amine, although others are also possible, on the base resin through standard reaction methods known in the art, such as any of the large number of reaction conditions developed for the formation of ester or amide bonds. Some linker moieties for the present invention are designed to allow for simultaneous cleavage from the resin with formation of the macrocycle in a process generally termed “cyclization-release.” (van Maarseveen, J. H. Comb. Chem. High Throughput Screen. 1998, 1, 185-214; James, I. W. Tetrahedron. 1999, 55, 4855-4946; Eggenweiler, H.-M. Drug Discovery Today 1998, 3, 552-560; Backes, B. J.; Ellman, J. A. Curr. Opin. Chem. Biol. 1997, 1, 86-93. Of particular utility in this regard for compounds of the invention is the 3-thiopropionic acid linker. Hojo, H.; Aimoto, S. Bull. Chem. Soc. Jpn. 1991, 64, 111-117; Zhang, L.; Tam, J. J. Am. Chem. Soc. 1999, 121, 3311-3320.)

Such a process typically provides material of higher purity as only cyclic products are released from the solid support and minimal contamination with the linear precursor occurs as would happen in solution phase. After sequential assembly of all the building blocks and tether into the linear precursor using known or standard reaction chemistry for the formation of ester or amide bonds, base-mediated intramolecular attack on the carbonyl attached to this linker by an appropriate nucleophilic functionality that is part of the tether building block results in formation of the amide or ester bond that completes the cyclic structure as shown (Scheme 1). An analogous methodology adapted to solution phase can also be applied as would likely be preferable for larger scale applications.

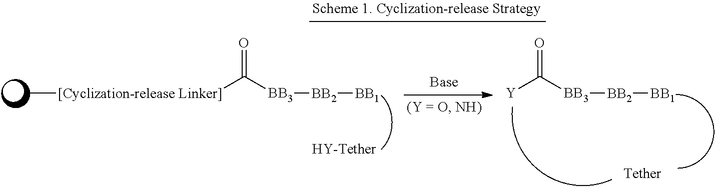

Although this description accurately represents the pathway for one of the methods of the present invention, the thioester strategy, another method of the present invention, that of ring-closing metathesis (RCM), proceeds through a modified route where the tether component is actually assembled during the cyclization step. However, in the RCM methodology as well, assembly of the building blocks proceeds sequentially, followed by cyclization (and release from the resin if solid phase). An additional post-cyclization processing step is required to remove particular byproducts of the RCM reaction, but the remaining subsequent processing is done in the same manner as for the thioester or analogous base-mediated cyclization strategy.

Moreover, it will be understood that steps including the methods provided herein may be performed independently or at least two steps may be combined. Additionally, steps including the methods provided herein, when performed independently or combined, may be performed at the same temperature or at different temperatures without departing from the teachings of the present invention.

Accordingly, the present invention provides methods of manufacturing the compounds of the present invention comprising (a) assembling building block structures, (b) chemically transforming the building block structures, (c) cyclizing the building block structures including a tether component, (d) removing protecting groups from the building block structures, and (e) optionally purifying the product obtained from step (d). In some embodiments, assembly of the building block structures may be sequential. In further embodiments, the synthesis methods are carried out using traditional solution synthesis techniques or solid phase chemistry techniques.

### A. General Synthetic Information

Reagents and solvents were of reagent quality or better and were used as obtained from commercial suppliers, including Sigma-Aldrich (Milwaukee, Wis., USA), Lancaster (part of Alfa Aesar, a Johnson Matthey Company, Ward Hill, Mass.), Acros Organics (Geel, Belgium), Alfa Aesar (part of Johnson Matthey Company, Ward Hill, Mass.), Fisher Chemical (part of Thermo Fisher, Fairlawn, N.J.), TCI America (Portland, Oreg.), Digital Specialty Chemicals (Toronto, ON, Canada), unless otherwise noted. DMF, DCM, DME and THF used are of DriSolv® (EM Science, E. Merck) or synthesis grade quality except for (i) deprotection, (ii) resin capping reactions and (iii) washing. NMP used for the amino acid (AA) coupling reactions is of analytical grade. DMF was adequately degassed by placing under vacuum for a minimum of 30 min prior to use. Analytical TLC was performed on pre-coated plates of silica gel 60F254 (0.25 mm thickness) containing a fluorescent indicator.

The term “concentrated/evaporated/removed under reduced pressure/vacuum” indicates evaporation utilizing a rotary evaporator under either water aspirator pressure or the stronger vacuum provided by a mechanical oil vacuum pump as appropriate for the solvent being removed. “Dry pack” indicates chromatography on silica gel that has not been pre-treated with solvent, generally applied on larger scales for purifications where a large difference in R_{f} exists between the desired product and any impurities. “Flash chromatography” refers to the method described as such in the literature (Still, W. C.; Kahn, M.; Mitra, A. J. Org. Chem. 1978, 43, 2923-2925) and is applied to chromatography on silica gel (230-400 mesh, EM Science) used to remove impurities some of which may be close in R_{f} to the desired material. Methods specific for solid phase chemistry are detailed separately.

### B. General Methods for Solid Phase Chemistry

These methods can be equally well applied for the synthesis of single compounds or small numbers of compounds, as well as for the synthesis of libraries of compounds of the present invention.

For solid phase chemistry, the solvent choice is important not just to solubilize reactants as in solution chemistry, but also to swell the resin. Certain solvents interact differently with the polymer matrix depending on its nature and can affect this swelling property. As an example, polystyrene (with DVB cross-links) swells best in nonpolar solvents such as DCM and toluene, while shrinking when exposed to polar solvents like alcohols. In contrast, other resins such as PEG-grafted ones like TentaGel, maintain their swelling even in polar solvents. For the reactions of the present invention, appropriate choices can be made by one skilled in the art. In general, polystyrene-DVB resins are employed with DMF and DCM common solvents. The volume of the reaction solvent required is generally 1-1.5 mL per 100 mg resin. When the term “appropriate amount of solvent” is used in the synthesis methods, it refers to this quantity. The recommended quantity of solvent roughly amounts to a 0.2 M solution of building blocks (linkers, amino acids, hydroxy acids; and tethers, used at 5 eq relative to the initial loading of the resin). Reaction stoichiometry was determined based upon the “loading” (represents the number of active functional sites, given as mmol/g) of the starting resin.

The reaction can be conducted in any appropriate vessel, for example round bottom flask, solid phase reaction vessel equipped with a fritted filter and stopcock, or Teflon-capped jar. The vessel size should be such that there is adequate space for the solvent, and that there is sufficient room for the resin to be effectively agitated taking into account that certain resins can swell significantly when treated with organic solvents. The solvent/resin mixture should fill about 60% of the vessel. Take note that all agitations for solid phase chemistry are best conducted with an orbital shaker (for example Form a Scientific, model 430, 160-180 rpm), except for those where scale makes use of gentle mechanical stirring more suitable, to ensure adequate mixing which is generally accepted to be important for a successful reaction.

The volume of solvent used for the resin wash is a minimum of the same volume as used for the reaction, although more is generally used to ensure complete removal of excess reagents and other soluble residual by-products. Each of the resin washes specified in the Examples should be performed for a duration of at least 5 min with agitation (unless otherwise specified) in the order listed. The number of washings is denoted by “nx” together with the solvent or solution, where n is an integer. In the case of mixed solvent washing systems, both are listed together and denoted solvent 1/solvent 2. The ratio of the solvent mixtures DCM/MeOH and THF/MeOH used in the washing steps is (3:1) in all cases. Other mixed solvents are as listed. After washing, drying in the “standard manner” means that the resin is dried first in air (1 h), and subsequently under vacuum (oil pump usually) until full dryness is attained (minimum 30 min, to 0/N).

### C. Amino Acids

Amino acids, Boc- and Fmoc-protected amino acids and side chain protected derivatives, including those of N-methyl and unnatural amino acids, were obtained from commercial suppliers [for example Advanced ChemTech (Louisville, Ky., USA), Anaspec (San Jose, Calif., USA), Astatech (Princeton, N.J., USA), Bachem (Bubendorf, Switzerland), Chemlmpex (Wood Dale, Ill., USA), Novabiochem (subsidiary of Merck KGaA, Darmstadt, Germany), PepTech (Burlington, Mass., USA), Synthetech (Albany, Oreg., USA)] or synthesized through standard methodologies known to those in the art. Ddz-amino acids were either obtained commercially from Orpegen (Heidelberg, Germany) or Advanced ChemTech (Louisville, Ky., USA) or synthesized using standard methods utilizing Ddz-OPh or Ddz-N₃. (Birr, C.; Lochinger, W.; Stahnke, G.; Lang, P. Justus Liebigs Ann. Chem. 1972, 763, 162-172.) Bts-amino acids were synthesized by known methods. (Vedejs, E.; Lin, S.; Klapara, A.; Wang, J. J. Am. Chem. Soc. 1996, 118, 9796-9797; WO 01/25257, WO 2004/111077) N-Alkyl amino acids, in particular N-methyl amino acids, are commercially available from multiple vendors (Bachem, Novabiochem, Advanced ChemTech, ChemImpex). In addition, N-alkyl amino acid derivatives were accessed via literature methods. (Hansen, D. W., Jr.; Pilipauskas, D. J. Org. Chem. 1985, 50, 945-950.) An improved synthesis of Fmoc-N-MeSer and Fmoc-N-MeThr has been reported. (Bahekar, R. H.; Jadav, P. A.; Patel, D. N.; Prajapati, V. M.; Gupta, A. A. Jain, M. R.; Patel, P. R. Tetrahedron. Lett. 2007, 48, 5003-5005.) alto-Threonine and β-hydroxyvaline can be synthesized by known procedures (Shao, H.; Goodman, M. J. Org. Chem. 1996, 61, 2582; Blaskovich, M. A.; Evindar, G.; Rose, N. G. W.; Wilkinson, S.; Luo, Y.; Lajoie, G., J. Org. Chem. 1998, 63, 3631; Dettwiler; J. E. Lubell, W. D. J. Org. Chem. 2003, 68, 177-179.) Chiral isomers of β-methylphenylalanines and β-methyltyrosines can be accessed using literature methods. (Dharanipragada, R.; Van Hulle, K.; Bannister, A.; Bear, S.; Kennedy, L.; Hruby, V. J. Tetrahedron 1992, 48, 4733-4748; Nicolas, E.; Russell, K. C.; Knollenberg, J.; Hruby, V. J. J. Org. Chem. 1993, 59, 7565-7571.) Similarly, chiral isomers of 4,4,4-trifluorothreonine with suitable protecting groups can be prepared by the enantioselective synthetic methods described in the literature. (Xiao, N.; Jinag, Z.-H.; Yu, Y. B. Biopolymers (Pept. Sci.) 2007, 88, 781-796.) Incorporation of the alto-isomer of L-threonine (2S,3S) could also be accomplished from the syn-L-isomer (2S,3R) based upon a similar transformation used in the synthesis of the natural product ustiloxin D (Wandless, T. J.; et al. J. Am. Chem. Soc. 2003, 115, 6864:6865.)

### D. Tethers

Certain tethers were obtained from the methods previously described in Intl. Pat. Appl. WO 01/25257, WO 2004/111077, WO 2005/012331, WO 2006/009645, WO 2006/009674 and U.S. Prov. Pat. Appl. 61/254,434.

Exemplary tethers (T) for the compounds of the invention include, but are not limited to, the following:

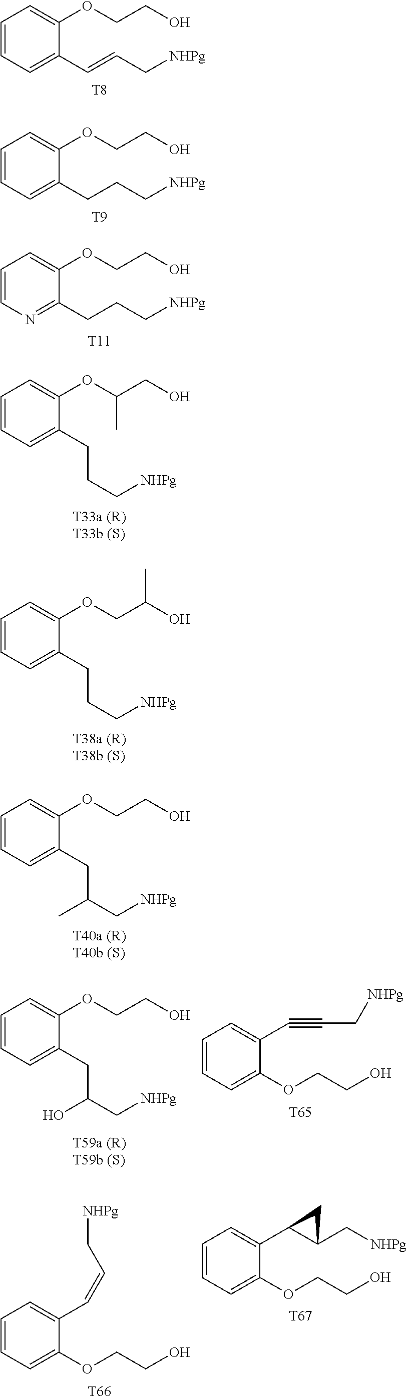

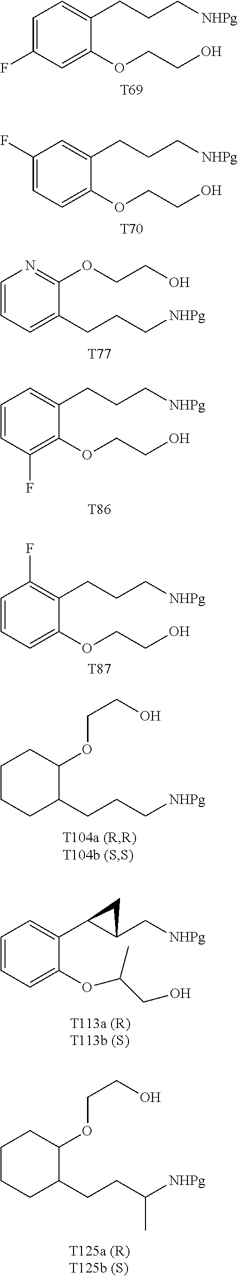

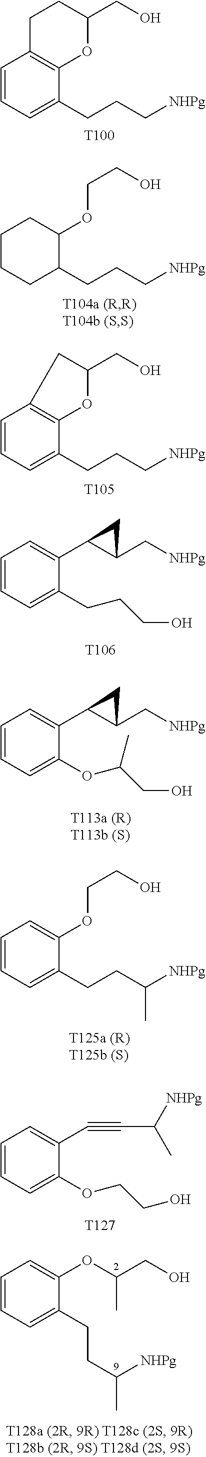

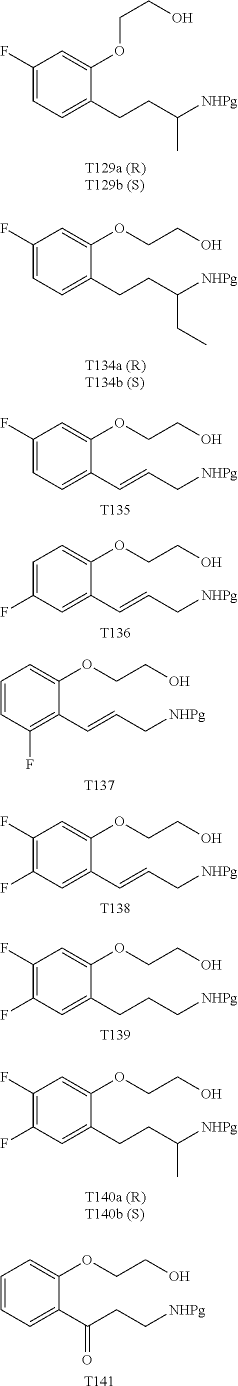

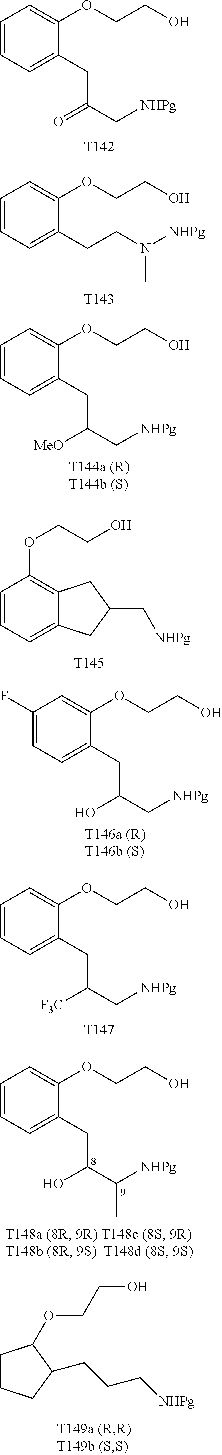

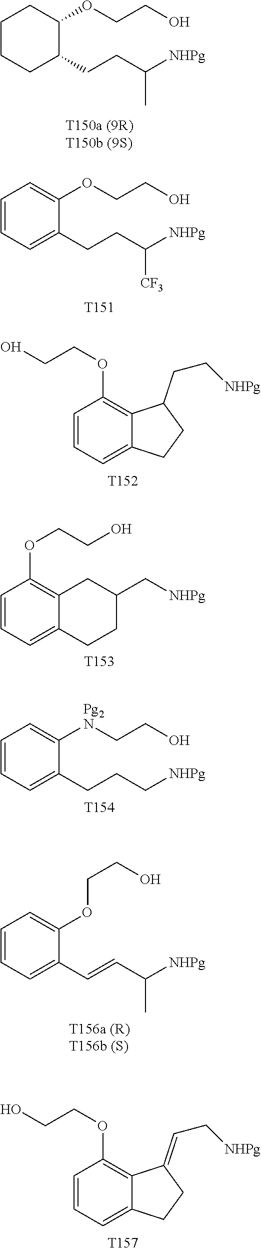

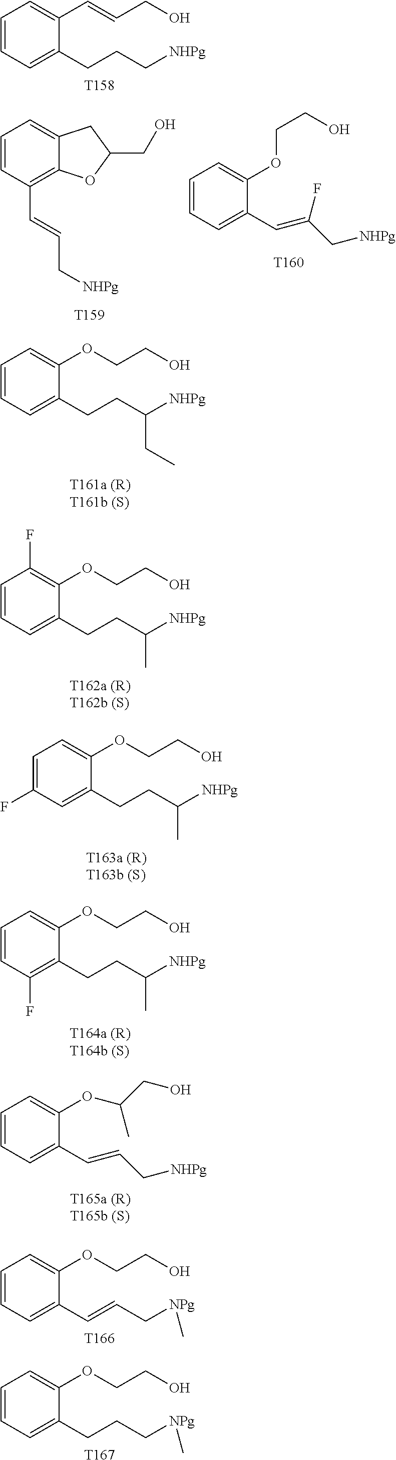

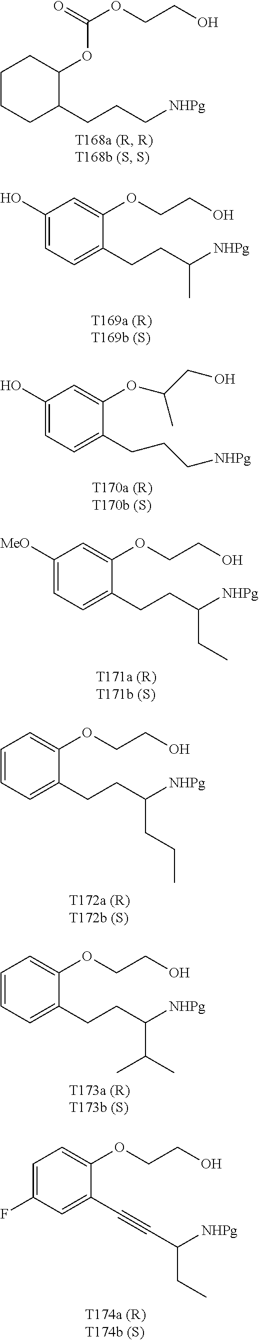

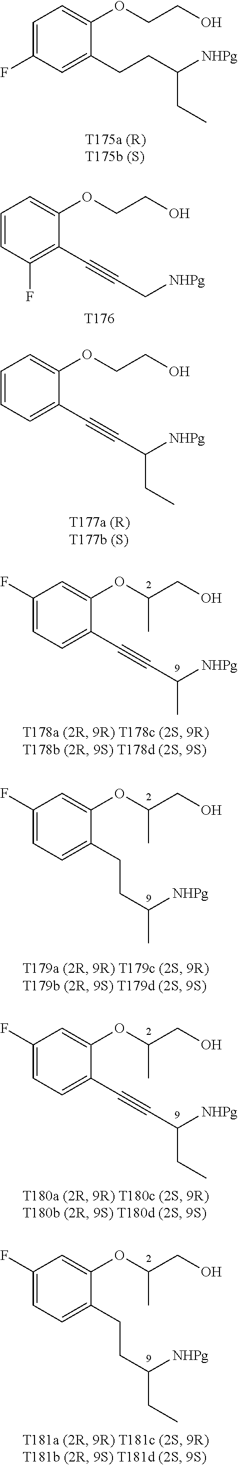

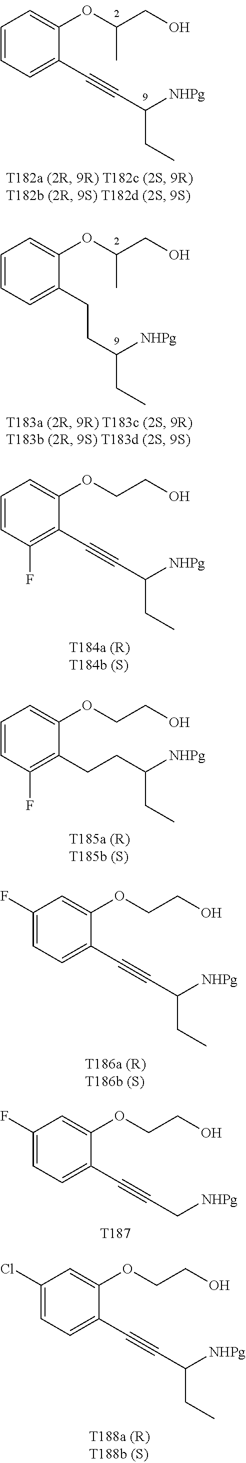

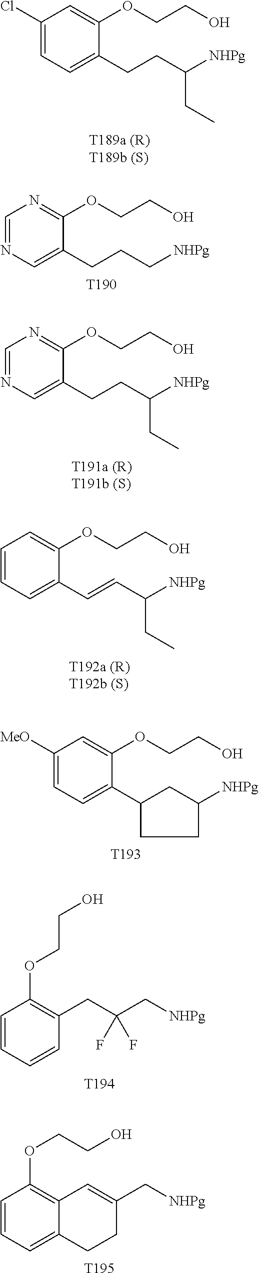

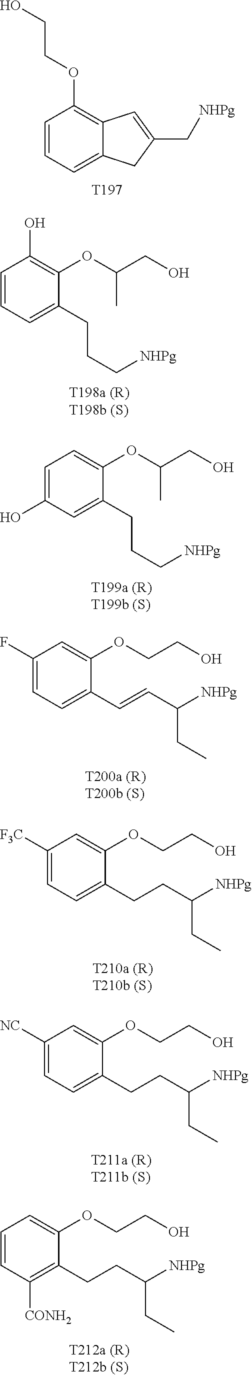

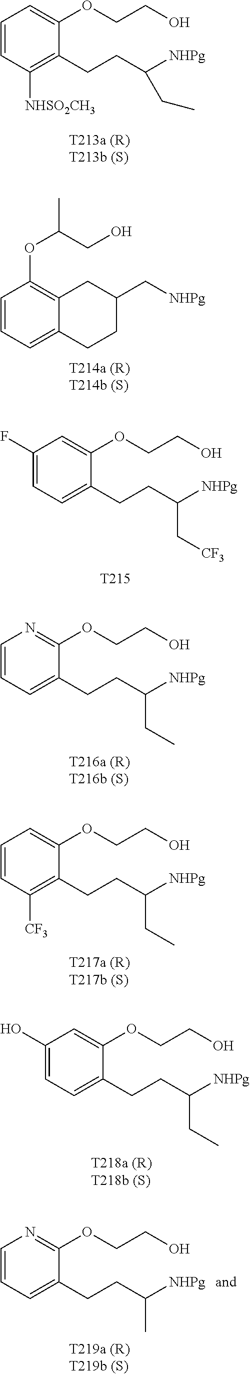

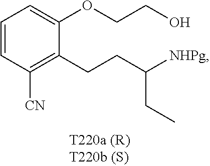

wherein Pg and Pg₂ are nitrogen protecting groups, such as, but not limited to, Boc, Fmoc, Cbz, Ddz and Alloc.

For representative syntheses of the new tether moieties disclosed herein, the routes presented in the Examples are employed. Although the routes described typically illustrate a specific protection strategy, other suitable protecting groups known in the art can also be employed.

### E. Solid Phase and Solution Phase Techniques

Specific solid phase techniques, including mixed solid-solution phase procedures, for the synthesis of the macrocyclic compounds of the invention have been described in Intl. Pat. Publ. WO 01/25257, WO 2004/111077, WO 2005/012331, WO 2005/012332, WO 2006/009645, WO 2006/009674, WO 2008/033328, WO 2008/130464 and U.S. Prov. Pat. Appl. 61/254,434 including purification procedures described in WO 2004/111077 and WO 2005/012331. Solution phase synthesis routes, including methods amenable to larger scale manufacture, were described in U.S. Patent Appl. Publ. Nos. 2006/025566 and US 2007/0021331.

### 3. Analytical Methods

Specific analytical techniques for the characterization of the macrocyclic compounds of the invention have been described in WO 01/25257, WO 2004/111077, WO 2005/012331 and WO 2005/012332.

¹H and ¹³C NMR spectra were recorded on a Varian Mercury 300 MHz spectrometer (Varian, Inc., Palo Alto, Calif.) and are referenced internally with respect to the residual proton signals of the solvent unless otherwise noted. ¹H NMR data are presented, using the standard abbreviations, as follows: chemical shift (δ) in ppm (multiplicity, integration, coupling constant(s)). The following abbreviations are used for denoting signal multiplicity: s=singlet, d=doublet, t=triplet, q=quartet, quint=quintet, b or br=broad, and m=multiplet. Information about the conformation of the molecules in solution can be determined utilizing appropriate two-dimensional NMR techniques known to those skilled in the art. (Martin, G. E.; Zektzer, A. S. Two-Dimensional NMR Methods for Establishing Molecular Connectivity: A Chemist's Guide to Experiment Selection, Performance, and Interpretation, John Wiley & Sons: New York, 1988, ISBN 0471187070.)

HPLC analyses were performed on a Waters Alliance® system 2695 running at 1 mL/min using an Xterra® MS C18 column (or comparable) 4.6×50 mm (3.5 μm) and the indicated gradient method. A Waters 996 PDA provided UV data for purity assessment (Waters Corporation, Milford, Mass.). For certain analyses, an LCPackings (Dionex Corporation, Sunnyvale, Calif.) splitter (50:40:10) allowed the flow to be separated in three parts. The first part (50%) was diverted to a mass spectrometer (Micromass® Platform II MS equipped with an APCI probe) for identity confirmation. The second part (40%) went to an evaporative light scattering detector (ELSD, Polymer Laboratories, now part of Varian, Inc.; Palo Alto, Calif., PLELS1000™) for purity assessment and the last portion (10%) went to a chemiluminescence nitrogen detector (CLND, Antek® Model 8060, Antek Instruments, Houston, Tex., part of Roper Industries, Inc., Duluth, Ga.) for quantitation and purity assessment. Each detector could also be used separately depending on the nature of the analysis required. Data was captured and processed utilizing the most recent version of the Waters Millennium® software package.

Representative standard HPLC conditions used for the analysis of compounds of the invention are presented below:

Typical Chromatographic Conditions

| Column: | XTerra RP18, 3.5 μm, 4.6 × 100 mm | |
|---|---|---|
| | (or equivalent) | |
| Detection (PDA): | 220-320 | nm |
| Column Temperature: | 35 ± 10° | C. |
| Injection Volume: | 10 | μL |
| Flow Rate: | 1 | mL/min |
| Run Time: | 20.0 | min |
| Data Acquisition Time: | 17.0 | min |

| | |
|---|---|
| Mobile Phase A: | Methanol (or Acetonitrile) |
| Mobile Phase B: | Water |
| Mobile Phase C: | 10% TFA in Water |

Gradient A4

| Time (min) | % A | % B | % C |
|---|---|---|---|
| 0.00 | 5.0 | 85.0 | 10.0 |
| 5.00 | 65.0 | 25.0 | 10.0 |
| 9.00 | 65.0 | 25.0 | 10.0 |
| 14.00 | 90.0 | 0.0 | 10.0 |
| 17.00 | 90.0 | 0.0 | 10.0 |
| 17.50 | 5.0 | 85.0 | 10.0 |
| 20.00 | 5.0 | 85.0 | 10.0 |

Gradient B4

| Time (min) | % A | % B | % C |
|---|---|---|---|
| 0.00 | 5.0 | 85.0 | 10.0 |
| 6.00 | 50.0 | 40.0 | 10.0 |
| 9.00 | 50.0 | 40.0 | 10.0 |
| 14.00 | 90.0 | 0.0 | 10.0 |
| 17.00 | 90.0 | 0.0 | 10.0 |
| 17.50 | 5.0 | 85.0 | 10.0 |
| 20.00 | 5.0 | 85.0 | 10.0 |

Preparative HPLC purifications were performed on final deprotected macrocycles using the Waters FractionLynx system, on an XTerra MS C18 column (or comparable) 19×100 mm (5 μm). The injections were done using an At-Column-Dilution configuration with a Waters 2767 injector/collector and a Waters 515 pump running at 2 mL/min. The mass spectrometer, HPLC, and mass-directed fraction collection are controlled via MassLynx software version 3.5 with FractionLynx. Fractions (13×125 mm tubes) shown by MS analysis to contain the product were evaporated under reduced pressure, most typically on a centrifugal evaporator system (Genevac HT-4, ThermoSavant Discovery, SpeedVac or comparable) or, alternatively, lyophilized. Compounds were then thoroughly analyzed by LC-MS-UV-ELSD-CLND analysis for identity confirmation, purity and quantity assessment.

Automated medium pressure chromatographic purifications were performed on an Isco CombiFlash 16× system with disposable silica or C18 cartridges that permitted up to sixteen (16) samples to be run simultaneously. MS spectra were recorded on a Waters Micromass Platform II or ZQ system. FIRMS spectra were recorded with a VG Micromass ZAB-ZF spectrometer. Chemical and biological information were stored and analyzed utilizing the Activityl)ase database software (IDBS, Guildford, Surrey, UK).

Analytical data for representative compounds of the invention are summarized in Table 2.

TABLE 2
Analytical Data for Representative Compounds of the Invention

| | Molecular | | |
|---|---|---|---|
| Compound | Formula | Molecular Weight | MS [(M + H)⁺] |
| 1300 | C32H44N4O5 | 564.7 | 565 |
| 1301 | C32H46N4O5 | 566.7 | 567 |
| 1302 | C32H46N4O5 | 566.7 | 567 |
| 1304 | C33H44N4O5 | 576.7 | 577 |
| 1305 | C28H36N4O6 | 524.6 | 525 |
| 1311 | C30H43N5O5 | 553.7 | 554 |
| 1313 | C32H44N4O5 | 564.7 | 565 |
| 1314 | C32H44N4O5 | 564.7 | 565 |
| 1315 | C32H44N4O5 | 564.7 | 565 |
| 1316 | C32H44N4O5 | 564.7 | 565 |
| 1317 | C31H40N4O5 | 548.7 | 549 |
| 1318 | C31H42N4O5 | 550.7 | 551 |
| 1319 | C30H40N4O5 | 536.7 | 537 |
| 1320 | C32H42N4O5 | 562.7 | 563 |
| 1323 | C32H44N4O5 | 564.7 | 565 |
| 1324 | C30H40N4O6 | 552.7 | 553 |
| 1325 | C31H41N4O5F | 568.7 | 569 |
| 1326 | C31H41N4O5F | 568.7 | 569 |
| 1327 | C32H41N4O5F3 | 618.7 | 619 |
| 1328 | C31H43N5O5 | 565.7 | 566 |
| 1329 | C28H40N6O5 | 540.7 | 541 |
| 1330 | C30H41N5O5 | 551.7 | 552 |
| 1331 | C29H40N4O6 | 540.7 | 541 |
| 1332 | C29H40N4O5S | 556.7 | 557 |
| 1333 | C31H43N5O4 | 549.7 | 550 |
| 1334 | C32H44N4O5 | 564.7 | 565 |
| 1335 | C33H46N4O4 | 562.7 | 563 |
| 1336 | C33H46N4O5 | 578.7 | 579 |
| 1337 | C33H46N4O5 | 578.7 | 579 |
| 1338 | C32H46N4O5 | 566.7 | 567 |
| 1339 | C31H44N4O6 | 568.7 | 569 |
| 1340 | C31H44N4O6 | 568.7 | 569 |
| 1341 | C31H41N4O5F | 568.7 | 569 |
| 1342 | C32H46N4O5 | 566.7 | 567 |
| 1343 | C31H43N4O5F | 570.7 | 571 |
| 1344 | C32H45N4O5F | 584.7 | 585 |
| 1345 | C31H42N4O5 | 550.7 | 551 |
| 1346 | C32H44N4O4 | 548.7 | 549 |
| 1347 | C32H46N4O5 | 566.7 | 567 |
| 1348 | C32H46N4O5 | 566.7 | 567 |
| 1349 | C32H43N4O5F3 | 620.7 | 621 |
| 1350 | C30H40N4O6 | 552.7 | 553 |
| 1351 | C31H42N4O6 | 566.7 | 567 |
| 1352 | C31H44N4O6 | 568.7 | 569 |
| 1353 | C31H42N4O5 | 550.7 | 551 |
| 1354 | C31H44N4O5 | 552.7 | 553 |
| 1355 | C31H42N4O5 | 550.7 | 551 |
| 1356 | C31H44N4O5 | 552.7 | 553 |
| 1357 | C31H44N4O5 | 552.7 | 553 |
| 1358 | C32H46N4O5 | 566.7 | 567 |
| 1359 | C31H44N4O6 | 568.7 | 569 |
| 1360 | C31H43N4O5F | 570.7 | 571 |
| 1361 | C31H44N4O5 | 552.7 | 553 |
| 1362 | C30H42N4O6 | 554.7 | 555 |
| 1363 | C30H41N4O5F | 556.7 | 557 |
| 1364 | C31H43N4O5F | 570.7 | 571 |
| 1365 | C30H41N4O6F | 572.7 | 573 |
| 1366 | C30H40N4O5F2 | 574.7 | 575 |
| 1367 | C31H42N4O5 | 550.7 | 551 |
| 1368 | C31H41N4O5F | 568.7 | 569 |
| 1369 | C32H43N4O5F | 582.7 | 583 |
| 1370 | C32H46N4O5 | 566.7 | 567 |
| 1371 | C32H46N4O6 | 582.7 | 583 |
| 1372 | C31H42N4O5F2 | 588.7 | 589 |
| 1373 | C32H46N4O5 | 566.7 | 567 |
| 1374 | C32H43N5O5 | 577.7 | 578 |
| 1375 | C33H45N5O5 | 591.7 | 592 |
| 1376 | C30H41N4O5F | 556.7 | 557 |
| 1377 | C30H41N4O5F | 556.7 | 557 |
| 1378 | C30H41N4O5F | 556.7 | 557 |
| 1379 | C31H43N4O5F | 570.7 | 571 |
| 1380 | C31H43N4O5F | 570.7 | 571 |
| 1381 | C31H43N4O5F | 570.7 | 571 |
| 1382 | C31H42N4O5 | 550.7 | 551 |
| 1383 | C31H43N4O6C1 | 603.1 | 603 |
| 1384 | C30H43N5O5 | 553.7 | 554 |
| 1385 | C29H41N5O5 | 539.7 | 540 |
| 1387 | C31H43N4O6F | 586.7 | 587 |
| 1388 | C32H44N4O5 | 564.7 | 565 |
| 1389 | C32H46N4O5 | 566.7 | 567 |
| 1390 | C32H46N4O5 | 566.7 | 567 |
| 1391 | C31H43N4O5F | 570.7 | 571 |
| 1392 | C31H42N4O5F2 | 588.7 | 589 |
| 1393 | C32H46N4O5 | 566.7 | 567 |
| 1394 | C31H44N4O5 | 552.7 | 553 |
| 1395 | C30H43N5O5 | 553.7 | 554 |
| 1396 | C31H40N4O5F2 | 586.7 | 587 |
| 1397 | C29H40N4O5 | 524.7 | 525 |
| 1398 | C32H46N4O5 | 566.7 | 567 |
| 1399 | C29H42N4O5S | 558.7 | 559 |
| 1400 | C31H43N4O5Cl | 587.1 | 587 |
| 1401 | C31H44N4O6 | 568.7 | 569 |
| 1402 | C31H41N4O5F3 | 606.7 | 607 |
| 1403 | C31H41N4O5F3 | 606.7 | 607 |
| 1404 | C32H46N4O5 | 566.7 | 567 |
| 1405 | C28H41N5O5S | 559.7 | 560 |
| 1406 | C33H44N5O5F | 609.7 | 610 |
| 1407 | C33H44N5O5F | 609.7 | 610 |
| 1408 | C32H44N6O5 | 592.7 | 593 |
| 1409 | C34H47N5O5 | 605.8 | 606 |
| 1411 | C31H41N4O5F3 | 606.7 | 607 |
| 1412 | C32H43N4O5F3 | 620.7 | 621 |
| 1413 | C34H45N5O5 | 603.8 | 604 |
| 1414 | C35H46N4O5 | 602.8 | 603 |
| 1415 | C35H46N4O5 | 602.8 | 603 |
| 1416 | C33H44N4O5S | 608.8 | 609 |
| 1417 | C29H42N4O5S | 558.7 | 559 |
| 1418 | C32H46N4O6 | 582.7 | 583 |
| 1419 | C30H39N4O5F | 554.7 | 555 |
| 1420 | C31H42N4O5F2 | 588.7 | 589 |
| 1421 | C31H42N4O5F2 | 588.7 | 589 |
| 1422 | C311142N4O5 | 550.7 | 551 |
| 1423 | C32H45N4O5F | 584.7 | 585 |
| 1424 | C32H45N4O5F | 584.7 | 585 |
| 1425 | C34H47N4O5F | 610.8 | 611 |
| 1426 | C36H49N5O5 | 631.8 | 632 |
| 1427 | C32H41N5O5 | 575.7 | 576 |
| 1428 | C33H44N5O5F | 609.7 | 610 |
| 1429 | C33H44N5O5F | 609.7 | 610 |
| 1430 | C31H42N4O5 | 550.7 | 551 |
| 1431 | C32H45N4O5F | 584.7 | 585 |
| 1432 | C30H39N4O5Cl | 571.1 | 571 |
| 1433 | C30H47N4O5Cl | 579.2 | 579 |
| 1434 | C31H42N4O5FCl | 605.1 | 605 |
| 1435 | C31H42N4O5FCl | 605.1 | 605 |
| 1436 | C31H42N4O5F2 | 588.7 | 589 |
| 1437 | C31H42N4O5F2 | 588.7 | 589 |
| 1438 | C30H38N4O5F2 | 572.6 | 573 |
| 1439 | C31H41N4O5F3 | 606.7 | 607 |
| 1440 | C31H41N4O5F3 | 606.7 | 607 |
| 1441 | C32H39N5O5 | 573.7 | 574 |
| 1442 | C33H42N5O5F | 607.7 | 608 |
| 1443 | C33H42N5O5F | 607.7 | 608 |
| 1444 | C32H45N4O6F | 600.7 | 601 |
| 1445 | C31H42N4O6 | 566.7 | 567 |
| 1446 | C32H45N4O6F | 600.7 | 601 |
| 1447 | C28H38N4O5S | 542.7 | 543 |
| 1448 | C29H41N4O5FS | 576.7 | 577 |
| 1449 | C29H41N4O5FS | 576.7 | 577 |
| 1450 | C31H43N4O5F | 570.7 | 571 |
| 1451 | C32H45N4O5F | 584.7 | 585 |
| 1453 | C31H44N4O6 | 568.7 | 569 |
| 1454 | C32H42N5O5F | 595.7 | 596 |
| 1455 | C33H44N5O5F | 609.7 | 610 |
| 1456 | C32H43N5O6 | 593.7 | 594 |
| 1457 | C31H43N4O5F | 570.7 | 571 |
| 1458 | C32H45N4O5F | 584.7 | 585 |
| 1459 | C31H44N4O6 | 568.7 | 569 |
| 1460 | C30H4ON4O5FCl | 591.1 | 591 |
| 1461 | C31H42N4O5FCl | 605.1 | 605 |
| 1462 | C30H41N4O6Cl | 589.1 | 590 |
| 1463 | C30H40N4O5F2 | 574.7 | 575 |
| 1464 | C31H42N4O5F2 | 588.7 | 589 |
| 1465 | C30H41N4O6F | 572.7 | 573 |
| 1466 | C30H39N4O5F3 | 592.6 | 593 |
| 1467 | C31H41N4O5F3 | 606.7 | 607 |
| 1468 | C30H40N4O6F2 | 590.7 | 591 |
| 1469 | C32H40N5O5F | 593.7 | 594 |
| 1470 | C33H42N5O5F | 607.7 | 608 |
| 1471 | C32H41N5O6 | 591.7 | 592 |
| 1472 | C31H43N4O6F | 586.7 | 587 |
| 1473 | C32H45N4O6F | 600.7 | 601 |
| 1474 | C31H44N4O7 | 584.7 | 585 |
| 1475 | C28H39N4O5FS | 562.7 | 563 |
| 1476 | C29H41N4O5FS | 576.7 | 577 |
| 1477 | C28H40N4O6S | 560.7 | 561 |
| 1478 | C32H45N4O5F | 584.7 | 585 |
| 1479 | C33H48N4O5 | 580.8 | 581 |
| 1480 | C32H45N4O5F | 584.7 | 585 |
| 1481 | C34H47N5O5 | 605.8 | 606 |
| 1482 | C33H48N4O5 | 580.8 | 581 |
| 1483 | C32H45N4O5Cl | 601.2 | 601 |
| 1484 | C32H44N4O5F2 | 602.7 | 603 |
| 1485 | C34H45N5O5 | 603.8 | 604 |
| 1486 | C30H38N4O5F2 | 572.6 | 573 |
| 1487 | C32H40N5O5F | 593.7 | 594 |
| 1488 | C30H38N4O5F2 | 572.6 | 573 |
| 1489 | C32H40N5O5F | 593.7 | 594 |
| 1490 | C30H38N4O5F2 | 572.6 | 573 |
| 1491 | C32H40N5O5F | 593.7 | 594 |
| 1492 | C30H37N4O5F3 | 590.6 | 591 |
| 1493 | C30H39N4O5F3 | 592.6 | 593 |
| 1494 | C32H39N5O5F2 | 611.7 | 612 |
| 1495 | C32H41N5O5F2 | 613.7 | 614 |
| 1496 | C31H41N4O5F3 | 606.7 | 607 |
| 1497 | C33H43N5O5F2 | 627.7 | 628 |
| 1498 | C30H42N5O5F | 571.7 | 572 |
| 1499 | C32H44N6O5 | 592.7 | 593 |
| 1500 | C31H43N4O6F | 586.7 | 587 |
| 1501 | C33H41N5O5 | 587.7 | 588 |
| 1502 | C33H45N5O6 | 607.7 | 608 |
| 1503 | C31H43N4O6F | 586.7 | 587 |
| 1504 | C33H45N5O6 | 607.7 | 608 |
| 1505 | C34H46N5O5F | 623.8 | 624 |
| 1506 | C33H47N4O5F | 598.7 | 599 |
| 1507 | C32H44N4O5FCl | 619.2 | 619 |
| 1508 | C32H43N4O5F3 | 620.7 | 621 |
| 1509 | C34H44N5O5F | 621.7 | 622 |
| 1510 | C32H45N4O5F | 584.7 | 585 |
| 1511 | C30H43N4O5FS | 590.8 | 591 |
| 1512 | C34H47N5O5 | 605.8 | 606 |
| 1513 | C32H45N4O5F | 584.7 | 585 |
| 1514 | C33H48N4O5 | 580.8 | 581 |
| 1515 | C32H44N4O5 | 564.7 | 565 |
| 1516 | C32H44N4O5 | 564.7 | 565 |
| 1517 | C32H44N4O5 | 564.7 | 565 |
| 1518 | C32H45N4O5F | 584.7 | 585 |
| 1519 | C29H40N5O5F | 557.7 | 558 |
| 1520 | C31H42N6O5 | 578.7 | 579 |
| 1521 | C33H48N4O6 | 596.8 | 597 |
| 1522 | C30H44N4O5S | 572.8 | 573 |
| 1523 | C32H42N5O6F | 611.7 | 612 |
| 1524 | C31H40N4O5F4 | 624.7 | 625 |
| 1525 | C33H42N5O5F3 | 645.7 | 646 |
| 1526 | C31H39N4O5F | 566.7 | 567 |
| 1527 | C32H45N4O6Cl | 617.2 | 617 |
| 1528 | C32H44N4O5F2 | 602.7 | 603 |
| 1529 | C33H47N4O5F | 598.7 | 599 |
| 1530 | C32H44N4O5F2 | 602.7 | 603 |
| 1531 | C33H47N4O6F | 614.7 | 615 |
| 1532 | C34H47N5O5 | 605.8 | 606 |
| 1533 | C30H39N4O6F | 570.7 | 571 |
| 1534 | C32H41N5O6 | 591.7 | 592 |
| 1535 | C31H45N5O4 | 551.7 | 552 |
| 1551 | C31H40N4O5 | 548.7 | 549 |
| 1552 | C31H40N4O5 | 548.7 | 549 |
| 1553 | C32H42N4O5 | 562.7 | 563 |
| 1554 | C31H40N4O5 | 548.7 | 549 |
| 1555 | C31H41FN4O5 | 568.7 | 569 |
| 1556 | C31H42N4O5 | 550.7 | 551 |
| 1558 | C30H37N4O4F | 536.6 | 537 |
| 1559 | C33H46N4O4 | 562.7 | 563 |
| 1560 | C33H46N4O5 | 578.7 | 579 |
| 1565 | C30H39N4O6F | 570.7 | 571 |
| 1566 | C32H41N5O6 | 591.7 | 592 |
| 1601 | C31H50N4O5 | 558.8 | 559 |
| 1602 | C31H50N4O5 | 558.8 | 559 |
| 1603 | C31H50N4O5 | 558.8 | 559 |
| 1604 | C30H48N4O5 | 544.7 | 545 |
| 1605 | C30H46N4O5 | 542.7 | 543 |
| 1606 | C32H50N4O7 | 602.8 | 603 |
| 1607 | C32H50N4O7 | 602.8 | 603 |
| 1608 | C31H45N4O7F | 604.7 | 605 |
| 1609 | C32H50N4O7 | 602.8 | 603 |
| 1610 | C32H50N4O7 | 602.8 | 603 |
| 1611 | C32H50N4O8 | 618.8 | 619 |
| 1612 | C29H46N4O7S | 594.8 | 595 |
| 1613 | C31H47N4O7Cl | 623.2 | 623 |
| 1614 | C31H46N4O7F2 | 624.7 | 625 |
| 1615 | C32H50N4O7 | 602.8 | 603 |
| 1616 | C32H47N5O7 | 613.7 | 614 |
| 1617 | C33H49N5O7 | 627.8 | 628 |
| 1618 | C30H47N5O7 | 589.7 | 590 |
| 1619 | C30H47N4O5F | 562.7 | 563 |
| 1620 | C32H49N5O5 | 583.8 | 584 |
| 1621 | C30H47N4O5Cl | 579.2 | 579 |
| 1622 | C30H46N4O5F2 | 580.7 | 581 |
| 1623 | C32H47N5O5 | 581.7 | 582 |
| 1624 | C30H47N4O5F | 562.7 | 563 |
| 1625 | C31H50N4O6 | 574.8 | 575 |
| 1626 | C28H46N4O5S | 550.8 | 551 |
| 1627 | C31H50N4O5 | 558.8 | 559 |
| 1628 | C31H50N4O5 | 558.8 | 559 |
| 1630 | C29H45N4O5F | 548.7 | 549 |
| 1631 | C31H47N5O5 | 569.7 | 570 |
| 1632 | C33H51N5O5 | 597.8 | 598 |
| 1633 | C31H49N4O5F | 576.7 | 577 |
| 1634 | C33H51N5O5 | 597.8 | 598 |
| 1635 | C31H49N4O5F | 576.7 | 577 |
| 1636 | C30H48N4O6 | 560.7 | 561 |
| 1655 | C30H48N4O6 | 560.7 | 561 |
| 1688 | C31H40N4O5 | 548.7 | 549 |
| 1689 | C31H41N4O5F | 568.7 | 569 |
| 1690 | C30H38N4O5F2 | 572.6 | 573 |
| 1691 | C30H37N4O5F | 552.6 | 553 |
| 1692 | C32H39N5O5 | 573.7 | 574 |
| 1693 | C32H38N5O5F | 591.7 | 592 |
| 1694 | C33H48N4O5 | 580.8 | 581 |
| 1695 | C33H48N4O5 | 580.8 | 581 |
| 1696 | C33H47N4O5F | 598.7 | 599 |
| 1697 | C35H49N5O5 | 619.8 | 620 |
| 1698 | C35H49N5O5 | 619.8 | 620 |
| 1699 | C31H43N4O5Cl | 587.1 | 587 |
| 1700 | C31H39N4O5Cl | 583.1 | 583 |
| 1701 | C32H42N4O5 | 562.7 | 563 |
| 1702 | C30H39N4O5F | 554.7 | 555 |
| 1703 | C35H46N5O5F | 635.8 | 636 |
| 1704 | C31H39N4O5Cl | 583.1 | 583 |
| 1705 | C34H47N4O5F | 610.8 | 611 |
| 1706 | C36H48N5O5F | 649.8 | 650 |
| 1707 | C36H44N5O5F | 645.8 | 646 |
| 1708 | C33H47N4O5F | 598.7 | 599 |
| 1709 | C34H42N5O5Cl | 636.2 | 636 |
| 1710 | C33H43N4O5Cl | 611.2 | 611 |
| 1711 | C31H39N4O5F | 566.7 | 567 |
| 1712 | C30H38N4O5 | 534.6 | 535 |
| 1713 | C34H41N5O5 | 599.7 | 600 |
| 1714 | C30H47N4O5Cl | 579.2 | 579 |
| 1715 | C31H49N4O5Cl | 593.2 | 593 |
| 1718 | C36H45N5O5 | 627.8 | 628 |
| 1719 | C35H46N5O5F | 635.8 | 636 |
| 1720 | C35H42N5O5F | 631.7 | 632 |
| 1721 | C34H46N4O5F2 | 628.7 | 629 |
| 1722 | C32H45N4O5F | 584.7 | 585 |
| 1723 | C32H44N4O5F2 | 602.7 | 603 |
| 1724 | C32H44N4O5F2 | 602.7 | 603 |
| 1725 | C34H46N5O5F | 623.8 | 624 |
| 1726 | C34H42N5O5F | 619.7 | 620 |
| 1727 | C35H49N5O6 | 635.8 | 636 |
| 1728 | C30H37N4O5Cl | 569.1 | 569 |
| 1729 | C31H39N4O5Cl | 583.1 | 583 |
| 1730 | C31H41N4O5Cl | 585.1 | 585 |
| 1731 | C31H41N4O5Cl | 585.1 | 585 |
| 1732 | C29H37N4O5Cl | 557.1 | 557 |
| 1733 | C32H43N4O5Cl | 599.2 | 599 |
| 1735 | C32H44N4O6 | 580.7 | 581 |
| 1736 | C32H44N4O5 | 564.7 | 565 |
| 1737 | C32H44N4O5 | 564.7 | 565 |
| 1738 | C31H41N4O5Cl | 585.1 | 585 |
| 1739 | C31H40N4O5FCl | 603.1 | 603 |
| 1740 | C31H40N4O5FCl | 603.1 | 603 |
| 1741 | C32H43N4O5Cl | 599.2 | 599 |
| 1742 | C32H45N4O5Cl | 601.2 | 601 |
| 1743 | C34H47N4O5F | 610.8 | 611 |
| 1744 | C34H47N4O5Cl | 627.2 | 627 |
| 1745 | C33H43N5O5 | 589.7 | 590 |
| 1746 | C33H45N4O5F | 596.7 | 597 |
| 1747 | C33H44N4O5F2 | 614.7 | 615 |
| 1751 | C32H45N4O6F | 600.7 | 601 |
| 1752 | C35H48N5O5F | 637.8 | 638 |
| 1753 | C32H44N4O5FCl | 619.2 | 619 |
| 1754 | C34H43N5O5 | 601.7 | 602 |
| 1755 | C34H42N5O5F | 619.7 | 620 |
| 1756 | C36H49N5O5 | 631.8 | 632 |
| 1757 | C31H44N5O4Cl | 586.2 | 586 |
| 1758 | C31H42N4O5FCl | 605.1 | 605 |
| 1759 | C32H41N4O5F | 580.7 | 581 |
| 1760 | C32H40N4O5F2 | 598.7 | 599 |
| 1761 | C31H40N4O5Cl2 | 619.6 | 619 |
| 1762 | C34H47N5O5 | 605.8 | 606 |
| 1763 | C34H47N4O5F | 610.8 | 611 |
| 1764 | C36H50N5O5F | 651.8 | 652 |
| 1768 | C31H41N4O5Cl | 585.1 | 585 |
| 1769 | C31H41N4O5F | 568.7 | 569 |
| 1770 | C33H42N5O5F | 607.7 | 608 |
| 1771 | C30H38N4O5F2 | 572.6 | 573 |
| 1772 | C30H39N4O5F | 554.7 | 555 |
| 1773 | C33H40N5O5F | 605.7 | 606 |
| 1774 | C34H46N5O5F | 623.8 | 624 |
| 1775 | C32H38N5O5F | 591.7 | 592 |
| 1776 | C33H46N4O5 | 578.7 | 579 |
| 1777 | C32H44N4O5 | 564.7 | 565 |
| 1778 | C32H42N4O5 | 562.7 | 563 |
| 1779 | C33H46N4O5 | 578.7 | 579 |
| 1780 | C31H42N4O5 | 550.7 | 551 |
| 1781 | C31H39N4O6Cl | 599.1 | 599 |
| 1782 | C33H44N4O6 | 592.7 | 593 |
| 1784 | C31H41N4O5Cl | 585.1 | 585 |
| 1785 | C32H45N4O5Cl | 601.2 | 601 |
| 1786 | C34H47N4O5Cl | 627.2 | 627 |
| 1787 | C36H49N5O5 | 631.8 | 632 |
| 1789 | C35H47N5O5 | 617.8 | 618 |
| 1790 | C33H46N4O6 | 594.7 | 595 |
| 1791 | C33H45N4O5F | 596.7 | 597 |
| 1792 | C33H45N4O5F | 596.7 | 597 |
| 1794 | C30H39N4O5Cl | 571.1 | 571 |
| 1795 | C32H44N4O6 | 580.7 | 581 |
| 1796 | C32H45N4O5F | 584.7 | 585 |
| 1797 | C35H48N4O5 | 604.8 | 605 |
| 1798 | C33H46N4O5 | 578.7 | 579 |
| 1799 | C31H40N4O5FCl | 603.1 | 603 |
| 1800 | C32H45N4O5Cl | 601.2 | 601 |
| 1801 | C33H44N5O5F | 609.7 | 610 |
| 1802 | C34H47N5O5 | 605.8 | 606 |
| 1803 | C34H45N5O5F2 | 641.7 | 642 |
| 1805 | C33H47N4O5F | 598.7 | 599 |
| 1806 | C34H46N5O5Cl | 640.2 | 640 |
| 1808 | C34H46N5O5F | 623.8 | 624 |
| 1809 | C33H40N5O5F | 605.7 | 606 |
| 1810 | C32H42N4O5 | 562.7 | 563 |
| 1811 | C31H41N4O5F | 568.7 | 569 |
| 1812 | C41H52N5O7FS | 777.9 | 778 |
| 1813 | C32H45N4O5Cl | 601.2 | 601 |
| 1814 | C32H44N4O5FCl | 619.2 | 619 |
| 1815 | C36H48N5O5F | 649.8 | 650 |
| 1824 | C30H43N4O6F | 574.7 | 575 |
| 1825 | C33H46N4O5 | 578.7 | 579 |
| 1826 | C33H46N4O5 | 578.7 | 579 |
| 1827 | C33H42N5O5F | 607.7 | 608 |
| 1829 | C33H43N4O5Cl | 611.2 | 611 |
| 1830 | C30H37N4O5Cl | 569.1 | 569 |
| 1831 | C31H41N4O5Cl | 585.1 | 585 |
| 1832 | C29H37N4O5Cl | 557.1 | 557 |
| 1834 | C32H44N4O6 | 580.7 | 581 |
| 1835 | C32H44N4O5 | 564.7 | 565 |
| 1836 | C32H44N4O5 | 564.7 | 565 |
| 1837 | C31H41N4O5Cl | 585.1 | 585 |
| 1838 | C31H40N4O5Cl2 | 619.6 | 619 |
| 1839 | C33H45N4O5F | 596.7 | 597 |
| 1840 | C32H46N4O5 | 566.7 | 567 |
| 1841 | C32H42N4O6 | 578.7 | 579 |
| 1842 | C33H43N4O6Cl | 627.2 | 627 |
| 1843 | C34H45N5O5 | 603.8 | 604 |
| 1844 | C34H45N5O5 | 603.8 | 604 |
| 1846 | C33H45N4O5F3 | 634.7 | 635 |
| 1847 | C31H45N5O5 | 567.7 | 568 |
| 1848 | C32H44N4O5 | 564.7 | 565 |
| 1849 | C32H44N4O5 | 564.7 | 565 |
| 1851 | C36H48N5O6F | 665.8 | 666 |
| 1852 | C32H45N4O5F | 584.7 | 585 |
| 1853 | C33H44N5O5F | 609.7 | 610 |
| 1854 | C31H43N4O5F | 570.7 | 571 |
| 1855 | C31H42N4O5F2 | 588.7 | 589 |
| 1856 | C32H42N4O5F4 | 638.7 | 639 |
| 1857 | C34H46N5O5F | 623.8 | 624 |
| 1858 | C32H43N4O5Cl | 599.2 | 599 |
| 1859 | C31H41N4O5Cl | 585.1 | 585 |
| 1860 | C33H43N4O5F3 | 632.7 | 633 |
| 1861 | C32H41N4O5F3 | 618.7 | 619 |
| 1862 | C31H43N4O5F | 570.7 | 571 |
| 1863 | C33H44N5O5F | 609.7 | 610 |
| 1864 | C33H47N5O6 | 609.8 | 610 |
| 1866 | C33H49N5O7S | 659.8 | 660 |
| 1867 | C33H44N4O5F4 | 652.7 | 653 |
| 1869 | C33H45N4O5F | 596.7 | 597 |
| 1870 | C33H44N4O5F2 | 614.7 | 615 |
| 1871 | C33H44N4O5FCl | 631.2 | 631 |
| 1872 | C32H42N4O5FCl | 617.2 | 617 |
| 1875 | C33H44N4O5FCl | 631.2 | 631 |
| 1876 | C31H37N4O5F | 564.6 | 565 |
| 1878 | C31H38N4O5 | 546.7 | 547 |
| 1879 | C31H37N4O5F | 564.6 | 565 |
| 1880 | C34H43N5O5 | 601.7 | 602 |
| 1881 | C33H44N4O5 | 576.7 | 577 |
| 1882 | C32H44N4O5 | 564.7 | 565 |
| 1883 | C32H43N4O5F | 582.7 | 583 |
| 1884 | C31H36N4O5F2 | 582.6 | 583 |
| 1885 | C34H43N5O5F4 | 677.7 | 678 |
| 1888 | C33H45N4O5F3 | 634.7 | 635 |
| 1889 | C33H45N5O5 | 591.7 | 592 |
| 1890 | C34H44N5O5F3 | 659.7 | 660 |
| 1891 | C35H46N5O5F3 | 673.8 | 674 |
| 1892 | C33H44N4O5F4 | 652.7 | 653 |
| 1893 | C32H42N5O5F | 595.7 | 596 |
| 1894 | C34H44N6O5 | 616.8 | 617 |
| 1895 | C34H45N5O5 | 603.8 | 604 |
| 1896 | C35H46N6O5 | 630.8 | 631 |
| 1897 | C33H44N5O5F | 609.7 | 610 |
| 1898 | C31H41N4O5F | 568.7 | 569 |
| 1899 | C31H42N4O5 | 550.7 | 551 |
| 1900 | C33H43N5O5 | 589.7 | 590 |
| 1901 | C33H44N4O5 | 576.7 | 577 |
| 1902 | C35H45N5O5 | 615.8 | 616 |
| 1903 | C32H43N4O5F | 582.7 | 583 |
| 1904 | C32H43N4O5Cl | 599.2 | 599 |
| 1905 | C32H45N5O5 | 579.7 | 580 |
| 1906 | C30H43N5O5 | 553.7 | 554 |
| 1907 | C31H43N5O5 | 565.7 | 566 |
| 1909 | C33H45N5O5 | 591.7 | 592 |
| 1911 | C32H43N4O5F3 | 620.7 | 621 |
| 1912 | C34H45N4O5F3 | 646.7 | 647 |
| 1913 | C33H44N4O5F2 | 614.7 | 615 |
| 1914 | C33H46N4O6 | 594.7 | 595 |
| 1916 | C32H42N4O5F2 | 600.7 | 601 |
| 1918 | C31H37N4O5F | 564.6 | 565 |
| 1919 | C31H36N4O5F2 | 582.6 | 583 |
| 1921 | C33H42N4O5F4 | 650.7 | 651 |
| 1922 | C34H46N6O5 | 618.8 | 619 |
| 1925 | C32H42N4O5F4 | 638.7 | 639 |
| 1927 | C34H47N5O6 | 621.8 | 622 |
| 1928 | C32H46N4O6 | 582.7 | 583 |
| 1929 | C30H37N4O5F | 552.6 | 553 |
| 1930 | C32H44N4O5 | 564.7 | 565 |

Notes
1. Molecular formulas and molecular weights are calculated automatically from the structure via ActivityBase software (ID Business Solutions, Ltd., Guildford, Surrey, UK).
2. M + H obtained from LC-MS analysis using standard methods.
3. All analyses conducted on material after preparative purification.

### 4. Biological Methods

The compounds of the present invention were evaluated for their ability to interact at the human ghrelin receptor utilizing a competitive radioligand binding assay, fluorescence assay, Aequorin functional assay or IP3 inverse agonist assay as described in the procedures below. Such methods can be conducted, if so desired, in a high throughput manner to permit the simultaneous evaluation of many compounds.

Specific assay methods for the human (GHS-R1a), swine and rat GHS-receptors (U.S. Pat. No. 6,242,199, Intl. Pat. Appl. Nos. WO 97/21730 and 97/22004), as well as the canine GHS-receptor (U.S. Pat. No. 6,645,726), and their use in generally identifying agonists and antagonists thereof are known.

Functional ghrelin antagonists can be identified utilizing the methods described in WO 2005/114180, while inverse agonists of the receptor can be assayed using the methods of WO 2004/056869.

Appropriate methods for determining the functional activity of compounds of the present invention that interact at the human ghrelin receptor are also described in the Examples below.

The in vivo efficacy of compounds of the present invention can be illustrated, for example, using animal models of obesity such as those described in the literature. (WO 2004/056869; Nakazato, M.; Murakami, N.; Date, Y.; et al. Nature 2001, 409, 194-198; Murakami, N.; Hayashida, T.; Kuroiwa, T.; et al. J. Endocrinol. 2002, 174, 283-288; Asakawa, A.; Inui, A.; Kaga, T.; et al. Gut 2003, 52, 947-952; Sun, Y.; Ahmed, S.; Smith, R. G. Mol. Cell. Biol. 2003, 23, 7973-7981; Wortley, K. E.; Anderson, K. D.; Garcia, K.; et al. Proc. Natl. Acad. Sci. USA 2004, 101, 8227-8232; Halem, H. A.; Taylor, J. E.; Dong, J. Z.; Shen, Y.; Datta, R.; Abizaid, A.; Diano, S.; Horvath, T.; Zizzari, P.; Bluet-Pajot, M.-T.; Epelbaum, J.; Culler, M. D. Eur. J. Endocrinol. 2004, 151, S71-S75; Helmling, S.; Maasch, C.; Eulberg, D.; et al. Proc. Natl. Acad. Sci USA 2004; 101, 13174-13179; Shearman, L. P.; Wang, S. P.; Helmling, S.; et al. Endocrinology 2006, 147, 1517-1526; Reuter, T. Y. Drug Disc. Today: Dis. Models 2007, 4, 3-8; Shafrir, E.; Ziv, E. Am. J. Physiol. 2009, 296, E1450-E1452.) Similarly, numerous animal models are available for studying the effects of these compounds in diabetes. (Nandi, A. et al. Physiol. Rev. 2004, 84, 623-647; Freude, S.; Schubert, M. Drug Disc. Today: Dis. Models 2007, 4, 9-16; Muniyappa, R.; Lee, S. Chen, H.; Quon, M. J. Am. J. Physiol. 2008, 294, E15-E26.)

### B1. Competitive Radioligand Binding Assay (Ghrelin Receptor)

The competitive binding assay at the human ghrelin receptor (GRLN, growth hormone secretagogue receptor, hGHS-R1a) was carried out analogously to assays described in the literature. (Bednarek M A et al. J. Med. Chem. 2000, 43, 4370-4376; Palucki, B. L. et al. Bioorg. Med. Chem. Lett. 2002, 11, 1955-1957.)

### Materials

Membranes (GHS-R/HEK 293) were prepared from HEK-293 cells stably transfected with the human ghrelin receptor (hGHS-R1a). These membranes were provided by PerkinElmer BioSignal (#RBHGHSM, lot#1887) and utilized at a quantity of 0.71 μg/assay point.
1. [¹²⁵I]-Ghrelin (PerkinElmer, #NEX-388); final concentration: 0.0070-0.0085 nM
2. Ghrelin (Bachem, #H-4864); final concentration: 1 μM
3. Multiscreen Harvest plates-GF/C (Millipore, #MAHFC1H60)
4. Deep-well polypropylene titer plate (Beckman Coulter, #267006)
5. TopSeal-A (PerkinElmer, #6005185)
6. Bottom seal (Millipore, #MATAH0P00)
7. MicroScint-0 (PerkinElmer, #6013611)
8. Binding Buffer: 25 mM Hepes (pH 7.4), 1 mM CaCl₂, 5 mM MgCl₂. 2.5 mM EDTA, 0.4% BSA

### Assay Volumes

Competition experiments were performed in a 300 μl filtration assay format.
1. 220 μL of membranes diluted in binding buffer
2. 40 μL of compound diluted in binding buffer
3. 40 μL of radioligand ([¹²⁵I]-Ghrelin) diluted in binding buffer
   Typical final test concentrations (N=1) for compounds of the present invention: 10, 1, 0.5, 0.2, 0.1, 0.05, 0.02, 0.01, 0.005, 0.002, 0.001 μM.

### Compound Handling

Compounds were provided frozen on dry ice at a stock concentration of 10 mM diluted in 100% DMSO and stored at −80° C. until the day of testing. On the test day, compounds were allowed to thaw at rt overnight and then diluted in assay buffer according to the desired test concentrations. Under these conditions, the maximal final DMSO concentration in the assay was 0.1%.

### Assay Protocol

In deep-well plates, 220 μL of diluted cell membranes (final concentration: 0.71 μg/well) were combined with 40 μL of either binding buffer (total binding, N=5), 1 μM ghrelin (non-specific binding, N=3) or the appropriate concentration of test compound (N=2 for each test concentration). The reaction was initiated by addition of 40 μL of [¹²⁵I]-ghrelin (final conc. 0.0070-0.0085 nM) to each well. Plates were sealed with TopSeal-A, vortexed gently and incubated at rt for 30 min. The reaction was arrested by filtering samples through Multiscreen Harvest plates (pre-soaked in 0.5% polyethyleneimine) using a Tomtec Harvester, washed 9 times with 500 μL of cold 50 mM Tris-HCl (pH 7.4, 4° C.), and then plates were air-dried in a fumehood for 30 min. A bottom seal was applied to the plates prior to the addition of 25 μL of MicroScint-0 to each well. Plates were than sealed with TopSeal-A and counted for 30 sec per well on a TopCount Microplate Scintillation and Luminescence Counter (PerkinElmer) using a count delay of 60 sec. Results were expressed as counts per minute (cpm).

Data were analyzed by GraphPad Prism (GraphPad Software, San Diego, Calif.) using a variable slope non-linear regression analysis. Kᵢ values were calculated using a K_{d} value of 0.01 nM for [¹²⁵I]-ghrelin (previously determined during membrane characterization). Dₘₐₓ values were calculated using the following formula:

$${D}_{max} = 1 - \frac{\begin{matrix}test concentration with maximal displacement - \\ non \text{-} specific binding\end{matrix}}{total binding - non \text{-} specific binding} \times 100$$

where total and non-specific binding represent the cpm obtained in the absence or presence of 1 μM ghrelin, respectively.

Results for the examination of representative compounds of the present invention using this method are presented in the Examples.

### B2. Fluorescence Functional Assay (Ghrelin Receptor)

### Equipment

1. ImageTrak Epi-Fluorescence system (Perkin-Elmer)
2. MultiDrop TiterTek
3. CO₂ incubators: 5% CO₂, humidified, 37° C.

### Materials

1. Hanks' BSS without phenol red (Life Technologies)
2. Hepes buffer
3. Probenecid (Sigma)
4. FLIPR Calcium-3 Assay Kit (Molecular Devices #R-8091)
5. Falcon cell culture 96-well black/clear bottom plates
6. 0.05% trypsin-EDTA
7. Cells: HEK293 cells expressing GHS-R1a receptor (Perkin-Elmer BioSignal) were grown in DMEM (Dulbecco's Modified Eagles Medium) with 10% FBS, 1% sodium pyruvate, 1% NEAA and 400 μg/mL geneticin
8. Ghrelin (reference agonist; Bachem, #H-4864)
9. [D-Lys³]-GHRP-6 (reference antagonist, Phoenix #031-22)
10. Assay buffer: HBSS-20 mM Hepes containing 2.5 mM probenecid and 0.1% BSA (bovine serum albumin); pH 7.4

### Compound Handling

Stock solutions of compounds (10 mM in 100% DMSO) were provided frozen on dry ice and stored at −80° C. prior to use. From the stock solution, mother solutions were made at a concentration of 100 μM by 100-fold dilution in 26% DMSO. Assay plates were then prepared by appropriate dilution in assay buffer.

Typical Final Test Concentrations (N=10) for Test Compounds (agonist):

### 1, 0.3, 0.1, 0.03, 0.01, 0.003, 0.001, 0.0003, 0.0001, 0.00003 μM.

Typical Final Test Concentrations (N=10) for Test Compounds (antagonist):

### 10, 3, 1, 0.3, 0.1, 0.03, 0.01, 0.003, 0.001, 0.0003 μM.

### Cell Preparation

Cells were maintained in culture as indicated above. The cells were harvested at a confluency of 70-90% the day before the experiment. Growth medium was removed and the cells rinsed briefly with PBS without Ca⁺² and Mg⁺². 0.05% Trypsin was added and the plates incubated at 37° C. for 5 min to detach the cells. DMEM medium supplemented with 10% FBS was added to inactivate the trypsin and determine the cell concentration. The inoculum was adjusted to a final concentration of 200 cells/μL and dispensed at 200 μL per well into a 96-well block plate. The plates were, incubated at 37° C. overnight. The cellular confluence must be between 70-95% on the day of the experiment.

### Assay Protocol

The plates were removed from the incubator and the media removed by inversion of the plates. Calcium-3 dye, 50 μL, was loaded and then incubated for 1 h at 37° C. The plate was again inverted and then 25 μL of assay buffer added. The plates were then transferred to the ImageTrak system for analysis. For agonist testing, after reading for ten (10) sec, 25 μL of 2× test compound or control was injected into the assay plate. Fluorescence was monitored for an additional 50 sec. A reading was taken every two (2) seconds for a total of 30 readings per assay point.

For antagonist testing, after reading for ten (10) sec, 12.5 μL of 3× test compound or control was injected into the assay plate and allowed to react for three (3) min. At that time, 4 nM ghrelin (corresponds to EC₈₀) was injected and fluorescence was monitored for an additional 60 sec. A reading was taken every two (2) seconds for a total of 125 readings per data point.

### Analysis and Expression of Results

For agonists, values obtained for each assay point reflect Max-Min of fluorescence readings where Max represents the maximal value of the 30 readings taken and Min represents the minimum value observed before injection of the compound from the first five readings. Concentration response curves were analyzed using GraphPad Prism (GraphPad Software, San Diego, Calif.) by non-linear regression analysis (sigmoidal dose-response). EC₅₀ values are calculated using GraphPad.

Eₘₐₓ values were calculated using the following formula:

$${E}_{max} = \frac{ \begin{matrix}\begin{matrix}counts at the concentration of \\ compound with maximum response -\end{matrix} \\ Basal\end{matrix}}{Ago ( {E}_{max} ) - Basal} \times 100$$

where Basal and Ago(Eₘₐₓ) represent the average counts obtained in the absence or presence of 1 μM ghrelin; respectively.

For antagonists, values obtained for each assay point reflect Max-Min of fluorescence readings where Max represents the maximal value obtained after injection of ghrelin at EC₈₀ and Min represents the minimum value observed before injection of the compound from the first five readings. Concentration response curves were analyzed using GraphPad Prism (GraphPad Software, San Diego, Calif.) by non-linear regression analysis (sigmoidal dose-response). IC₅₀ values are calculated using GraphPad.

Iₘₐₓ values were calculated using the following formula:

$${I}_{max} = \frac{\begin{matrix}\begin{matrix}counts at concentration of compound \\ with maximum response -\end{matrix} \\ Ago ( {EC}_{80} )\end{matrix}}{Basal - Ago ( {EC}_{80} )} \times 100$$

where Basal and Ago(EC₈₀) represent the average counts obtained in the absence or presence of 5 nM ghrelin at the second addition step, respectively.

### B3. Aequorin Functional Assay (Ghrelin Receptor)

The functional activity of compounds of the invention found to bind to the GRLN (GHS-R1a) receptor can be determined using the method described below. (LePoul, E.; et al. J. Biomol. Screen. 2002, 7, 57-65; Bednarek, M. A.; et al. J. Med. Chem. 2000, 43, 4370-4376; Palucki, B. L.; et al. Bioorg. Med. Chem. Lett. 2001, 11, 1955-1957.).

### Materials

Membranes were prepared using AequoScreen™ (Perkin-Elmer, Waltham, Mass.) cell lines expressing the human ghrelin receptor (cell line ES-410-A; receptor accession #60179). This cell line is constructed by transfection of the human ghrelin receptor into CHO-K1 cells co-expressing G_{α16} and the mitochondrially targeted Aequorin (Ref #ES-WT-A5).
1. Ghrelin (reference agonist; Bachem, #H-4864)
2. Assay buffer: DMEM (Dulbecco's Modified Eagles Medium) containing 0.1% BSA (bovine serum albumin; pH 7.0.
3. Coelenterazine (Molecular Probes, Leiden, The Netherlands)
   Typical final concentrations for test compounds, which are tested in duplicate:
   0.1, 0.3, 1, 3, 10, 30, 100, 300, 1000, 3000 nM

### Compound Handling

Stock solutions of compounds (10 mM in 100% DMSO) were typically provided frozen on dry ice and stored at −20° C. prior to use. From the stock solution, mother solutions were made at a concentration of 1 mM by dilution to a final concentration of 30% DMSO. Assay plates were then prepared by appropriate dilution in DMEM medium containing 0.1% BSA. Under these conditions, the maximal final DMSO concentration in the assay was <0.6%.

### Cell Preparation

AequoScreen™ cells were collected from culture plates with Ca²⁺ and Mg²⁺-free phosphate buffered saline (PBS) supplemented with 5 mM EDTA, pelleted for 2 minutes at 1000×g, re-suspended in DMEM—Ham's F12 containing 0.1% BSA at a density of 5×10⁶ cells/ml and incubated at room temperature for at least 4 h in the presence of 5 μM coelenterazine. After loading, cells were diluted with assay buffer to a concentration of 5×10⁵ cells/ml.

### Assay Protocol

For agonist testing, 50 μl of the cell suspension were mixed with 50 μl of the appropriate concentration of test compound or ghrelin (reference agonist) in 96-well plates (duplicate samples). Ghrelin (reference agonist) is tested at several concentrations concurrently with the test compounds in order to validate the experiment. The emission of light resulting from receptor activation in response to ghrelin or test compounds was recorded using the Hamamatsu Functional Drug Screening System 6000 reader (Hamamatsu Photonics K. K., Japan).

For antagonist testing, an approximate EC₈₀ concentration of ghrelin (i.e. 3.7 nM; 100 μL) was injected onto 100 μL of the cell suspension containing the test compounds (duplicate samples) after approximately 15 min incubation after the end of agonist testing and the consequent emission of light resulting from receptor activation was measured as described in the paragraph above. [D-Lys³]-GHRP-6 was used a s a reference antagonist.

To standardize the emission of recorded light (determination of the “100% signal”) across plates and across different experiments, some of the wells contained 100 μM digitonin, a saturating concentration of ATP (20 μM) and a concentration of ghrelin equivalent to the EC₅₀ obtained during test validation. Plates also contained the reference agonist and/or antagonist at a concentration equivalent to the EC₈₀ obtained during the test validation.

### Analysis and Expression of Results

Results are expressed as Relative Light Units (RLU). Concentration response curves were analyzed using GraphPad Prism (GraphPad Software, San Diego, Calif.) by non-linear regression analysis (sigmoidal dose-response) based on the equation E=Eₘₐₓ/(1+EC₅₀/C)n where E is the measured RLU value at a given agonist concentration (C), Eₘₐₓ is the maximal response, EC₅₀ is the concentration producing 50% stimulation and n is the slope index. For agonist testing, results for each concentration of test compound were expressed as percent activation relative to the signal induced by ghrelin at a concentration equal to the EC₈₀ (i.e. 3.7 nM). EC₅₀, Hill slope and % Eₘₐₓ values are reported.

For antagonist testing, results for each concentration of test compound were expressed as percent inhibition relative to the signal induced by ghrelin at a concentration equal to the EC₈₀. Results for representative compounds of the invention are presented in the Examples.

### B4. Ghrelin Receptor Inverse Agonist Assay

The inverse agonist activity at the ghrelin receptor for compounds of the invention can be determined using the methods described in Intl. Pat. Appl. Publ. No. WO 2004/056869 and Hoist, B.; Cygankiewicz, A.; Halkjaer, T.; Ankersen, A.; Schwartz, T. W. Mol. Endocrinol. 2003, 17, 2201-2210. As an alternative, a phosphatidyl inositol hydrolysis assay as reported in the literature (Jensen, A. A., et al. J. Biol. Chem. 2000, 275, 29547-29555) can be utilized to assess the inverse agonist activity of compounds of the invention. In addition, the functional receptor assay termed Receptor Sepection and Amplification Technology (R-SAT), as described in U.S. Pat. Nos. 5,707,798; 5,912,132; 5,955,281 and International Pat. Appl. Publ. No. WO 2007/079239, can be used to evaluate these compounds.

In addition, the following method can be utilized to assay for inverse agonist activity. (Thomsen, W.; et al. Curr. Opin. Biotechnol. 2005, 16, 655-665; Tozawa-Takahashi F; et al., 11th SBS Annual Conference. September 2005, Geneva; Trinquet, E.; Fink, M.; Bazin, H.; et al. Anal. Biochem. 2006, 358, 126-135; Bergsdorf, C.; Kropp-Goerkis, C.; Kaehler, I.; Ketscher, L.; Boerner, U.; Parczyk, K.; Bader, B. Assay Drug Dev. Technol. 2008, 6, 39-53.)

### Cell Stimulation:

1. Remove culture medium from the plate by inversion.
2. Add 70 μl of compound/well.
3. Incubate 30 min at 37° C.
4. Stop the reaction by adding 15 μl of lysis buffer/well.
5. Add 15 μl of d2/well.
6. Add 15 μl of Anti-IP1 cryptate/well.
7. Incubate 1 h at room temp on an orbital shaker at 100 RPM.
8. Read the fluorescence in a plate reader (Tecan GeniosPro or similar)

The above sequence was performed using the HTRF IP-one kit (CisBio cat#62P1APEC). For the simultaneous assay of multiple test compounds, 96-well plates can be utilized in this assay (white plate with flat-bottom well, Falcon #353296). These were seeded overnight with 100 000 of HEK-GHSR1 stable cells/well.
Wells A1 and A2 of each plate are used as negative control (wells without d2).
   Compounds are typically tested in replicate at the following concentrations:

### 0, 1 nM, 10 nM, 30 nM, 100 nM, 300 nM, 1 μM, 10 μM.

### Compound Dilution:

Compounds are stored at 10 mM in 100% DMSO.

1^{st} dilution 1/10 in 100% DMSO (1 mM final concentration).

2^{nd} dilution 1/10 in H₂O (0.1 mM final concentration).

Other dilutions are performed in a 96-well plate in stimulation buffer.

The results for representative compounds of the invention are provided in the Examples.

### B5. Plasma Protein Binding

The pharmacokinetic and pharmacodynamic properties of drugs are largely a function of the reversible binding of drugs to plasma or serum proteins such as albumin and α₁-acid glycoprotein. In general, only unbound drug is available for diffusion or transport across cell membranes, and for interaction at the pharmacological target. On the other hand, drugs with low plasma protein binding generally have large volumes of distribution and rapid clearance since only unbound drug is available for glomerular filtration and, in some cases, hepatic clearance. Thus, the extent of plasma protein binding can influence efficacy, distribution and elimination. The ideal range for plasma protein binding is in the range of 87-98% for most drug products.

Protein binding studies were performed using human plasma. Briefly, 96-well microplates were used to incubate various concentrations of the test article for 60 min at 37° C. A concentration of 10 μM was a typical selection to be employed in this study. Bound and unbound fractions are separated by equilibrium dialysis, where the concentration remaining in the unbound fraction is quantified by LC-MS or LC-MS-MS analysis. Drugs with known plasma protein binding values such as quinine (˜35%), warfarin (˜98%) and naproxen (˜99.7%) were used as reference controls.

Results for representative compounds of the invention are summarized in the Table 3.

TABLE 3
Human Plasma Protein Binding for
Representative Compounds of the Invention

| Compound | Binding (%) |
|---|---|
| 1453 | 75.7 |
| 1503 | 77.9 |
| 1505 | 96.4 |
| 1688 | 90.9 |
| 1692 | 98.2 |
| 1700 | 99.1 |
| 1703 | 99.5 |
| 1707 | 99.6 |
| 1711 | 97.4 |
| 1712 | 97.6 |
| 1720 | 99.3 |
| 1726 | 99.8 |
| 1751 | 97.4 |
| 1754 | 99.4 |
| 1755 | 99.3 |
| 1777 | 95.8 |
| 1778 | 92.4 |
| 1780 | 93.9 |
| 1843 | 92.1 |
| 1848 | 79.3 |
| 1876 | 95 |
| 1878 | 87.3 |
| 1903 | 84.1 |

### B6. Assay for Cytochrome P450 Inhibition

Cytochrome P450 enzymes are implicated in the phase I metabolism of drugs. The majority of drug-drug interactions are metabolism-based and, moreover, these interactions typically involve inhibition of cytochrome P450s. Six CYP450 enzymes (CYP1A2, CYP2C8, CYP2C9, CYP2C19, CYP2D6 and CYP3A4) appear to be commonly responsible for the metabolism of most drugs and the associated drug-drug interactions. Assays to determine the binding of compounds of the invention to the various metabolically important isoforms of cytochrome P450 metabolizing enzymes are commercially available, for example NoAb BioDiscoveries (Mississaugua, ON, Canada) and Absorption Systems (Exton, Pa., USA). As well, a number of appropriate methods have been described or reviewed in the literature. (White, R. E. Ann. Rev. Pharmacol. Toxicol. 2000, 40, 133-157; Li, A. P. Drug. Disc. Today 2001, 6, 357-366; Turpeinen, M.; Korhonen, L. E. Tolonen, A.; et al. Eur. J. Pharm. Sci. 2006, 29, 130-138.)

The key aspects of the experimental method were as follows:
1. Assay was performed on microsomes (Supersomes®, BD Gentest, Becton-Dickinson) prepared from insect cells expressing individual human CYP-450 subtypes, specifically:
   CYP subtypes: 1A2, 2A6, 2B6, 2C8, 2C9, 2C19, 2D6, 2E1, 3A4
   Two substrates are typically tested for CYP-3A4 as this enzyme exhibits complex inhibition kinetics
2. Assays monitored, via fluorescence detection, the formation of a fluorescent metabolite following incubation of the microsomes with a specific CYP substrate.
3. Compounds of the present invention were tested in duplicate samples at eight test concentrations using 3-fold serial dilutions (concentration range of 0.0457 to 100 μM).
4. For each CYP-450 enzyme, a specific inhibitor was tested in duplicate at eight concentrations as a positive control.
5. The concentration of the inhibitor or test compound that inhibited metabolite formation by 50% (IC₅₀) was calculated by non-linear regression analysis of the % inhibition vs. log concentration (M) curve.

Results for representative compounds of the invention are summarized in Tables 4a and 4b below.

TABLE 4a
Cytochrome P450 Binding for
Representative Compounds of the Invention

| Compound | IC₅₀ CYP 3A4^{a} (μM) | IC₅₀ CYP 2D6^{b} (μM) |
|---|---|---|
| 1453 | 13.4 | 9.21 |
| 1503 | 14.3 | 55.8 |
| 1505 | 0.7 | 2.1 |
| 1688 | 8.5 | 20.2 |
| 1777 | 6 | 11.8 |
| 1778 | 7.7 | 21.1 |
| 1780 | 6 | 35.7 |
| 1843 | 6.5 | 7.7 |
| 1848 | 8 | 14.1 |
| 1876 | 8.5 | 23.1 |
| 1878 | 11.6 | 45.3 |
| 1903 | 9 | 8 |
| 1918 | 16.3 | 8.1 |
| 1929 | — | 25.7 |

^{a}Nifedipine used as substrate (midazolam was also employed)
^{b}Dextromethorphan used as substrate

No binding was obtained to the other CYP subtypes tested up to the highest concentration tested (100 μM).

TABLE 4b
Cytochrome P450 Binding for
Representative Compounds of the Invention

| Compound | IC₅₀ CYP 3A4^{a} (μM) | IC₅₀ CYP 2D6^{b} (μM) |
|---|---|---|
| 1318 | 3.9 | >5 |
| 1319 | 8.0 | 19.1 |
| 1324 | >5 | >5 |
| 1325 | >3.1 | >5 |
| 1326 | 2.2 | >5 |
| 1327 | >17.7 | >25 |
| 1340 | 17.2 | 13.3 |
| 1350 | 5.7 | 7.9 |
| 1358 | 1.6 | >20 |
| 1375 | 8.8 | >20 |
| 1390 | 6.9 | >20 |
| 1399 | 2.3 | >20 |
| 1413 | 1.0 | 14.7 |
| 1418 | 0.9 | 14.5 |
| 1428 | 0.8 | 9.1 |
| 1429 | 0.7 | >20 |
| 1432 | 1.2 | 5.2 |
| 1433 | 2.6 | 3.1 |
| 1453 | 3.7 | 9.2 |
| 1479 | 1.5 | >20 |
| 1490 | 1.4 | 6.3 |
| 1501 | 1.5 | >20 |
| 1504 | 1.4 | 12.7 |
| 1515 | 1.1 | >8 |
| 1526 | 1.4 | >20 |
| 1601 | 2.6 | >5 |
| 1619 | 0.6 | >20 |
| 1693 | 2.2 | — |
| 1712 | 5.8 | — |
| 1720 | 1.6 | — |
| 1729 | 1.9 | — |
| 1730 | 1.6 | — |
| 1732 | 2.9 | — |
| 1919 | 11.5 | — |

^{a}Mmidazolam used as substrate (nifedipine was also employed)
^{b}Dextromethorphan used as substrate
— indicates not tested with this subtype

### B7. Determination of Caco-2 Permeability

The Caco-2 cell line, derived from a human colorectal carcinoma, has become an established in vitro model for the prediction of drug absorption across the human intestine. (Sun, D.; Yu, L. X.; Hussain, M. A.; Wall, D. A.; Smith, R. L.; Amidon, G. L. Curr. Opin. Drug Discov. Devel. 2004, 7, 75-85; Bergstrom, C. A. Basic Clin. Pharmacol. Toxicol. 2005, 96, 156-61; Balimane, P. V.; Han, Y. H.; Chong, S. AAPS J. 2006, 8, E1-13; Shah, P.; Jogani, V.; Bagchi, T.; Misra, A. Biotechnol. Prog. 2006, 22, 186-198.) When cultured on semi-permeable membranes, Caco-2 cells differentiate into a highly functionalized epithelial barrier with remarkable morphological and biochemical similarity to the small intestinal columnar epithelium. Fully differentiated cell monolayers can be used to assess the membrane transport properties of novel compounds. In addition, the apparent permeability coefficients (Pₐₚₚ) obtained from Caco-2 cell transport studies have been shown to reasonably correlate with human intestinal absorption.

Assays to determine the permeability of compounds of the invention utilizing Caco-2 cells are commercially available, for example NoAb BioDiscoveries (Mississaugua, ON, Canada) and Absorption Systems (Exton, Pa., USA).

Alternatively, parallel artificial membrane permeability assays (PAMPA) can be utilized to assess intestinal permeability. (Avdeef, A. Expert Opin. Drug. Metab. Toxicol. 2005, 1, 325-342.)

### Method

Permeability across the Caco-2 cell layer was determined by growing the cells on a membrane placed between two (donor and acceptor) chambers. Drug candidates are typically added to the apical (A) side of the cell layer and their appearance in the basolateral (B) side is measured over incubation time. Permeability in this direction represents intestinal absorption. Permeability may also be determined from the basolateral to the apical side of the Caco-2 cell. A higher apical to basolateral Pₐₚₚ, compared to the basolateral to apical Pₐₚₚ, is indicative of carrier-mediated transport. P-gp mediated transport is suggested when a higher basolateral to apical Pₐₚₚ is observed relative to the apical to basolateral Pₐₚₚ.

Permeability (10 μM) for compounds of the invention in the apical to basolateral and basolateral to apical direction were tested in duplicate. Samples will be collected from the donor and acceptor chambers at the beginning (0 min) and following 60 min of incubation at 37° C. and stored frozen at −70° C. until bioanalysis. Samples for each test compound generated from the Caco-2 permeability assay were further analyzed by LC-MS-MS. The permeability of [³H]-mannitol and [³H]-propranolol were determined in parallel as controls.

The permeability coefficient (Pₐₚₚ) of each compound and radiolabeled standard was determined using the following equation:

$${P}_{app} = \frac{d Q}{d T} \times 1 / {C}_{i} \times 1 \text{/} A$$

where dQ/dT represents the permeability rate, Cᵢ denotes the initial concentration in the donor compartment, and A represents the surface area of the filter. Cᵢ is determined from the mean concentration of duplicate samples taken prior to addition to the donor compartment. Permeability rates were calculated by plotting the cumulative amount of compound measured in the acceptor compartment over time and determining the slope of the line by linear regression analysis. The duplicate and mean apical to basolateral and basolateral to apical Pₐₚₚ's were reported for each compound and standard.

To further ascertain the involvement of Pgp, use of an inhibitor of Pgp, for example cyclosporine A, can be utilized in this evaluation and the results with and without inhibitor compared. Results for representative compounds of the invention are summarized in Table 5.

TABLE 5
Caco-2 Permeability of Representative Compounds of the Invention

| | Without P-gp | | | With P-gp | | |
|---|---|---|---|---|---|---|
| | inhibitor | | | inhibitor^{b} | | |
| | A to B | | Efflux ratio | A to B | | Efflux ratio |
| | Mean Pₐₚₚ | | Papp B2A/Pₐₚₚ | Mean Pₐₚₚ | | Pₐₚₚ B2A/Pₐₚₚ |
| Compound | (×10⁶ cm/s) | B to A | A2B | (×10⁶ cm/s) | B to A | A2B |
| 1503 | 0.11 | 12 | 109 | 0.581 | 4.96 | 8.53 |
| 1505 | 0.091^{a} | 26.7^{a} | 299^{a} | 3.00^{a} | 16.3^{a} | 5.69^{a} |
| 1688 | 0.131 | 41.8 | 318 | 4.86 | 13.4 | 2.75 |
| 1777 | 0.274 | 53.5 | 195 | 5.02 | 9.94 | 1.98 |
| 1778 | 0.193 | 32.7 | 169 | 2.15 | 16.4 | 7.6 |
| 1780 | 0.099 | 29.5 | 297 | 1.99 | 13.1 | 6.59 |
| 1843 | 0.142 | 13.4 | 95 | 0.727 | 9.78 | 13.5 |
| 1848 | 0.266 | 64.2 | 241 | 11.3 | 24.9 | 2.21 |
| 1876 | 0.097 | 28 | 288 | 1.65 | 14.1 | 8.52 |
| 1878 | 0.144 | 21.7 | 151 | 1.34 | 8.66 | 6.45 |
| 1903 | 0.291 | 58.9 | 203 | 11.9 | 28.5 | 2.39 |
| 1918 | 0.112 | 42.6 | 380 | 8.32 | 18.4 | 2.21 |
| 1929 | 0.171 | 36.9 | 216 | 3.33 | 18.4 | 5.54 |

^{a}Average of three experiments
^{b}Cyclosporin A

### B8. Metabolic Stability in Human Liver Microsomes

The liver is the primary site for phase I (oxidation) and phase II (glucuronidation) enzymatic activity responsible for xenobiotic metabolism. Human liver microsomes are used as in vitro screen of metabolic activity for candidate drugs. Similar studies can be run with microsomes from other species, such as those used for in vivo studies, to determine any significant species differences in the stability profile. The aim of this study was to measure the broad-spectrum metabolic stability of representative compounds of the invention. The key aspects of the experimental design are summarized below:
Human liver microsomes (mixed pool of 15 male and female donors) were purchased from In Vitro Technologies (Baltimore, Md.).
   Microsomes characterized for phase I (Cyp2A6, 2D6, 2E1, 1A2, 2C19, 3A4, 4A) and phase II (glucuronidation) enzymatic activity.
Assays are performed using a final concentration of 0.8 mg/mL of microsomes in 100 mM potassium phosphate buffer (1.5 mM NADPH, 8 mM MgCl₂, pH 7.4, 37° C.).
Compounds are tested in duplicate samples at a single concentration of 5 μM (0.05% DMSO).
Test articles are incubated with the microsomes at 37° C. Samples are collected at 0; 15 and 30 min.
Test compounds and propranolol (positive control) samples are analyzed in comparison to an internal standard by LC/MS/MS.
Metabolic half-life is determined by non-linear regression analysis of the metabolic degradation curve obtained by the % compound remaining at time=0, 15 and 30 min.
   Results obtained for representative compounds of the invention are presented in Table 6.

TABLE 6
Metabolic Stability of Representative Compounds
of the Invention in Human Liver Microsomes

| | HLM |
|---|---|
| Compound | (μL/min/mg protein) |
| 1319 | 26.5 |
| 1371 | 30.5 |
| 1372 | 60.8 |
| 1373 | 31 |
| 1374 | 35.8 |
| 1375 | 58.4 |
| 1376 | 32.2 |
| 1377 | 65.5 |
| 1378 | 42.9 |
| 1390 | 53 |
| 1391 | 16.6 |
| 1392 | 23.6 |
| 1393 | 46.6 |
| 1400 | 54.2 |
| 1412 | 35.4 |
| 1418 | 32.2 |
| 1432 | 25.1 |
| 1451 | 10.4 |
| 1458 | 9.8 |
| 1473 | 14.2 |
| 1479 | 15.7 |
| 1482 | 34.6 |
| 1486 | 8.7 |
| 1492 | 14.6 |
| 1501 | 23.6 |
| 1503 | 20.9 |
| 1505 | 51.5 |
| 1506 | 7.5 |
| 1512 | 24.7 |
| 1515 | 54.5 |
| 1526 | 13.6 |
| 1528 | 35.5 |
| 1529 | 13.8 |
| 1565 | 7.8 |
| 1619 | 69.3 |
| 1630 | 38.7 |
| 1688 | 41.4 |
| 1690 | 21.8 |
| 1691 | 53.7 |
| 1692 | 121 |
| 1693 | 83.8 |
| 1699 | 85.2 |
| 1700 | 32.8 |
| 1701 | 40.4 |
| 1702 | 14.1 |
| 1703 | 44.8 |
| 1704 | 33.5 |
| 1707 | 27.3 |
| 1712 | 58.2 |
| 1713 | 48.8 |
| 1718 | 43.6 |
| 1719 | 23.4 |
| 1720 | 23.2 |
| 1723 | 64.3 |
| 1725 | 66.5 |
| 1726 | 41.5 |
| 1729 | 54.8 |
| 1730 | 61.9 |
| 1732 | 52.2 |
| 1737 | 83.9 |
| 1738 | 53.2 |
| 1739 | 26.1 |
| 1740 | 28.3 |
| 1742 | 157.4 |
| 1745 | 117.0 |
| 1746 | 38.6 |
| 1751 | 109.6 |
| 1752 | 14.3 |
| 1754 | 43.7 |
| 1755 | 47.8 |
| 1758 | 90.4 |
| 1759 | 40.6 |
| 1760 | 34.8 |
| 1761 | 77.0 |
| 1762 | 73.4 |
| 1763 | 15.6 |
| 1777 | 39.6 |
| 1778 | 58.1 |
| 1780 | 25.3 |
| 1843 | 33.7 |
| 1848 | 60.7 |
| 1876 | 30.9 |
| 1878 | 34.7 |
| 1903 | 47.9 |
| 1918 | 14.3 |

### B9. Pharmacokinetic Analysis

The pharmacokinetic (PK) behavior of compounds of the invention and their pharmaceutical compositions can be ascertained by methods well known to those skilled in the art and can be used to investigate the pharmacokinetic parameters (elimination half-life, total plasma clearance, etc.) for intravenous, subcutaneous and oral administration of these substances. (Wilkinson, G. R. “Pharmacokinetics: The Dynamics of Drug Absorption, Distribution, and Elimination” in Goodman & Gilman's The Pharmacological Basis of Therapeutics, Tenth Edition, Hardman, J. G.; Limbird, L. E., Eds., McGraw Hill, Columbus, Ohio, 2001, Chapter 1.) See also U.S. Pat. Nos. 7,476,653; 7,491,695; Intl. Pat. Appl. WO 2008/033328 and U.S. Patent Appl. Publ. 2008/0194672. As an example, compound 1505 has the PK profile below.

| Compound | t_{1/2}(min) | Cl (mL/min/kg) | Oral F(%) |
|---|---|---|---|
| 1505 | 64 | 23 | 18 |

The determination of PK parameters for additional representative compounds of the invention is presented in the Examples.

### B10. Ex-vivo Potency Evaluation on the Rat Stomach Fundus

This method is employed to provide an additional evaluation of the potency of compounds of the invention as ghrelin antagonists by treatment of rat stomach fundus strips in an organ bath ex vivo in the presence or absence of electrical field stimulation (EFS). Ghrelin peptide is used to simulate the activity of the tissue and then the ability of varying concentrations of the test compound investigated.

### Method

Fundus strips (approximately 0.4×1 cm) were cut from the stomach of adult male Wistar rats parallel to the circular muscle fibers. They were placed between two platinum ring electrodes, 1 cm apart (Radnoti, ADlnstruments, USA) in 10 ml tissue baths containing Krebs solution bubbled with 5% CO₂ in O₂ and maintained at 37° C. Tissues were suspended under 1.5 g resting tension. Changes of tension were measured isometrically with force transducers and recorded with a PowerLab 8/30 data acquisition system (ADlnstruments, USA). Tissues were allowed to equilibrate for 60 min during which time bath solutions were changed every 15 min.

EFS was achieved by applying 0.5 ms pulses, 5 Hz frequency, at a maximally effective voltage of 70 V. EFS was applied for 30 sec at 3 min intervals for a 30 min initial period. This initial period was separated by a 5 mM interval with wash out of the bath solution. Then, a second period of stimulation was started. After obtaining consistent EFS-evoked contractions (after three or four 30 sec stimulations), the effects of ghrelin as a positive control, ghrelin with test compounds at various concentrations (for example 0.01-10 μM), L-NAME (300 μM, as control) or their respective vehicles, applied non-cumulatively, on responses to EFS were studied over a 30 min period. Responses to the agents were measured and expressed as % of the mean of three or four pre-drug responses to EFS. All compounds were dissolved at 1 mM in distilled water or MeOH, as stock solutions.

### Results

IC₅₀ values for the inhibition of ghrelin-induced contractility by representative compounds of the invention are presented in Table 7.

TABLE 7
Inhibition of Rat Fundus Contractility by
Representative Compounds of the Invention

| Compound | IC₅₀ (nM) |
|---|---|
| 1315 | 75 |
| 1319 | 72 |
| 1325 | 29 |
| 1364 | 200 |
| 1391 | 65 |
| 1392 | 4 |
| 1400 | 360 |
| 1453 | 2900 |
| 1503 | 650 |
| 1505 | 12.5 |
| 1688 | 0.1 |
| 1712 | 3.4 |
| 1777 | 7.8 |
| 1778 | 12 |
| 1780 | 12.1 |
| 1843 | 2.3 |
| 1848 | 15 |
| 1876 | 60 |
| 1878 | 30 |
| 1903 | 1.6 |
| 1918 | 26 |
| 1929 | 2 |

### B11. Effects of 14-Day Administration of Representative Compounds of the Invention on Glucose Homeostasis and Metabolism in Wistar Rats

### Objective

The objective of the study was to determine the effects of representative compounds of the invention on body weight, food and water consumption, glucose homeostasis and tolerance as well as serum lipids, plasma insulin and selected metabolic parameters in the liver, adipose tissue and skeletal muscle in male Wistar rats, when administered subcutaneously or orally for 14 d.

### Test Protocol

On experimental day −7 animals were stratified according to body weight into an appropriate number of groups of 6 animals each (main study animals). Test compounds were administered as solutions either subcutaneously or orally. The dose volume was 2 or 3 mL/kg. Timing of dosing was done to ensure maximal exposure during the dark phase, particularly at the beginning of the dark phase when feeding is more intense.

| | | | Total | | | |
|---|---|---|---|---|---|---|
| | | | daily | | Dosage | |
| | | Dose | dose | Dose | Volume | |
| Group | Test | (mg/ | (mg/ | Concentration | (mL/kg/ | No of |
| No. | Article | kg) | kg) | (mg/mL) | day) | Animals |
| 1 | Vehicle | 0 | 0 | 0 | 2 | 6 |
| | Control | | | | | |
| | (s.c.) | | | | | |
| 2 | Test cmpd | 40 | 40 | 20 | 2 | 6 |
| | 1 (s.c.) | | | | | |
| 3 | Test cmpd | 40 | 80 | 13.3 | 3 (b.i.d.) | 6 |
| | 2 (s.c.) | | | | | |
| 4 | Test cmpd | 50 | 100 | 25 | 2 (b.i.d.) | 6 |
| | 3 (p.o.) | | | | | |
| 5 | Test cmpd | 10 | 10 | 5 | 2 | 6 |
| | 4 (p.o.) | | | | | |

Vehicle (Group 1) as well as two of the test compounds (Group 2 and Group 5) were administered once daily 1 h prior to the end of the light phase (5:00 P.M.) while other test compounds (Group 3 and Group 4) were administered twice daily at 10:00 A.M. and 5:00 P.M. Other dose levels and concentrations can be investigated similarly.

### In-life Observations

For the study animals, the data collected from study Days −7 to 16 are reported. Body weights were recorded for all animals daily starting on Day −7 prior to initiation of dosing, at the time of group assignment and throughout the study period as well as terminally prior to necropsy. Food and water intake was measured every 3 days at 8:00 A.M. starting on Day 1 prior to initiation of dosing and throughout the treatment period.

From all animals of Groups 1-5 (main study animals), blood was collected by a cardiac puncture on experimental Day 16 at 08:00 AM for the determination of plasma concentrations of glucose, as well as serum concentration of free fatty acids, triacylglycerol, and total cholesterol. One drop of blood (˜20 μL) was used for plasma glucose on Accu-Chek Aviva glucometers (Roche Diagnostics, Indianapolis, Ind.). For the other parameters, one (1) mL blood was collected in pre-cooled serum separation clotting activator tubes (Sarstedt). The blood was centrifuged at 2500 rpm (4° C., 10 min), serum transferred into non-coated tubes and stored at −80° C. until analysis.

### Blood Sampling for Oral Glucose Tolerance Test (OGTT)

The oral glucose tolerance test was carried out in all animals of Groups 1-5 around 8:00 A.M. The test was performed on half of the animals from each group on experimental day 3 and on the other half of the animals from each group on experimental day 4. The same procedure was repeated on experimental days 14 and 15. Animals were subjected to an overnight fast (food removed the day before at 5:00 PM). Blood samples of approximately 250 μL each for plasma glucose and insulin measurements were collected into EDTA coated tubes (K2-EDTA microtainer tubes, Becton Dickinson) from a tail vein, at 0, 15, 30, 60, and 120 min on experimental days 3, 4, 13 and 14, after oral administration of 1.5 g/kg glucose (dextrose, Sigma Aldrich, 450 mg/ml dosing solution). The glucose solution was administered by oral gavage via a stainless steel feeding needle (18×2″, Popper @ Sons, cat. # 20068-642, VWR). While glucose concentrations were determined from a drop of blood of this sample (Accu-Chek Aviva glucometers, Roche Diagnostics), the remainder was centrifuged at 4000 rpm for 10 min. at 4° C., and the resulting plasma transferred into non-coated tubes and stored at −80° C. for insulin determination.

### Analytical Methods

Plasma insulin was measured in duplicate for each data point and animal with an HTRF insulin detection kit (Cat. No. 62INSPEB, CisBio, USA). Plasma glucose was measured using ACCU-CHEK Aviva glucometers (Roche Diagnostics). Serum cholesterol and triglycerides was measured using standard enzyme assay kits (TGs: cat. # 11488872216, Roche Diagnostics; Chol: cat. # 11489232216, Roche Diagnostics). The measurements will be performed on a Hitachi 912 analyzer. Serum free fatty acids (FFA) was measured in duplicates using a commercially available colorimetric enzyme assay kit (HR series NEFA-HR (2) kit, WAKO Chemicals).

### Data Evaluation and Statistics

All data was entered into Excel 2003 spreadsheets and subsequently subjected to relevant statistical analyses (GraphPad Prism, GraphPad Software, San Diego, Calif.). Results are presented as mean±SD (standard deviation) unless otherwise stated. Statistical evaluation of the data is carried out using one-way analysis of variance (ANOVA) with appropriate post-hoc analysis between control and treatment groups in cases where statistical significance was established.

### B12. Suppression of Feeding Response

As another approach to determining the in vivo activity of compounds of the invention, suppression of the feeding response in fasted rats can be performed as described in the literature (Sartor, O.; et al. Endocrinology 1985, 117, 1441-1447).

### B13. Effects of Acute Administration of Representative Compounds of the Invention on Glucose Homeostasis and Metabolism in Male Zucker Fatty Rats

### Objective

The objective of this study is to determine the acute effects of test compounds on body weight change, food and water consumption and glucose homeostasis in male Zucker fatty rats 24 h post-dose and after 3 days of subcutaneous administration. The same parameters are evaluated 24 h post-dose and after 3 days of administration of test compound by the intraperitoneal route. The male Zucker fatty rat has been selected as an insulin resistance and genetically defined obesity model which is sensitive to the effect of different insulin sensitizers in acute as well as in chronic settings.

### Animals

Rats were individually housed in rodent cages with soft wood bedding on the bottom and equipped with water bottles. All individual cages were clearly labeled with a cage card indicating study number, group, animal number and dose level. Each animal was uniquely identified by an animal number. The animal number was designated the day the animals arrived at the animal facility. The animal room environment was controlled (targeted ranges: temperature 22±2° C.; relative humidity 50±10%; light/dark cycle: 12 hours light, 12 hours dark, lights on from 06:00 AM to 06:00 PM). A regular rodent diet (Charles River 5075 rodent chow, Purina Mills, Canada) was provided to the animals ad libitum, after food weighing. Municipal tap water was provided to the animals ad libitum via water bottles. Fresh tap water was provided after water bottle weighing.

An acclimation period of approximately 4 days for all groups was allowed between the receipt of animals and the start of treatment to accustom the rats to the laboratory environment. On experimental day −3, animals were stratified according to body weight into an appropriate number of groups of 4 animals each.

### Test Protocol

Test compounds were administered, as solutions, subcutaneously or intraperitoneally at the targeted doses indicated below. The dose volume was 3 mL/kg. Groups 2, 3 and 5 were dosed once daily around 7:00 a.m., while groups 1, 4, 6 and 7 were closed twice daily (b.i.d) at around 7:00 a.m. and 4:00 p.m. On Day 1 on half of the animals (Subset A) and on Day 2 on the other half (Subset B), an OGTT was performed 2 hrs post-dosing (around 9:00 a.m.). The OGTT was repeated the same way on Days 3 and 4.

| | | | Total | | | |
|---|---|---|---|---|---|---|
| | | | daily | Dose | Dosage | |
| | | Dose | dosage | Concentration | Volume | No of |
| Group No. | Test Article | (mg/kg) | (mg/kg) | (mg/mL) | (mL/kg/day) | Animals |
| 1 | Vehicle | 0 | 0 | 0 | 3 × 2 | 4 |
| (Fatty) | control (b.i.d, | | | | | |
| | s.c.) | | | | | |
| 2 | Vehicle | 0 | 0 | 0 | 3 | 4 |
| (Fatty) | control (s.c.) | | | | | |
| 3 | Test cmpd 1 | 40 | 40 | 13.3 | 3 | 4 |
| (Fatty) | in vehicle | | | | | |
| | control (s.c.) | | | | | |
| 4 | Test cmpd 2 | 40 | 80 | 13.3 | 3 × 2 | 4 |
| (Fatty | in vehicle | | | | | |
| | control (b.i.d, | | | | | |
| | s.c.) | | | | | |
| 5 | Lean control | 0 | 0 | 0 | 3 | 4 |
| (Lean) | (vehicle | | | | | |
| | treated) (s.c.) | | | | | |
| 6 | Vehicle | 0 | 0 | 0 | 3 × 2 | 4 |
| (Fatty) | control (b.i.d, | | | | | |
| | i.p.) | | | | | |
| 7 | Test cmpd 2 | 40 | 80 | 13.3 | 3 × 2 | 4 |
| (Fatty) | in vehicle | | | | | |
| | control (b.i.d, | | | | | |
| | i.p.) | | | | | |

Other dose levels and concentrations can be investigated similarly.

For the study animals, the data collected from study Days −3 to 4 were reported. Body weights were recorded for all animals on Day −3 prior to initiation of dosing, at the time of group assignment and daily throughout the study period (Day 1-4). 24 h food and water intake was measured (around 12:00 p.m.) on Day 2 and 4 (Subset A) and Day 3 and 5 (Subset B). On Day 1, animals from groups 3, 4 and 7 were sampled for blood (˜100 μl) 15 min, 30 min, 1 hr and 2 hrs post-dosing (just before the OGTT) for PK analysis. Blood was centrifuged at 4000 rpm for 10 min. at 4° C., and the resulting plasma transferred into non-coated tubes and stored at −80° C. until analysis. On Day 3, only a 2 hrs post-dosing (just before the OGTT) blood sample was taken for PK analysis.

An oral glucose tolerance test was carried out in animals of all groups on Day 1 and 2 (half of the animals) as well as on day 3 and 4 (other half of the animals). This was done 2 hrs post-dosing. Animals were subjected to an overnight fast (food removed the day before at 5:00 PM). To this effect blood samples of approximately 20 μL each for plasma glucose and 230 μL for plasma insulin measurements were collected into EDTA coated tubes (K₂-EDTA microtainer tubes, Becton Dickinson) from a tail vein, at 0 (pre-glucose), 15, 30, 60, and 120 min on experimental day 3 and 4 (blood sampling for glucose only on Day 1 and 2, no insulin) after oral administration of 1.5 g/kg glucose (dextrose, Sigma Aldrich, 450 mg/ml dosing solution). The glucose solution was administered by oral gavage via a stainless steel feeding needle (18×2″, Popper @ Sons, cat. # 20068-642, VWR). While glucose concentrations will be determined from a drop of blood (Accu-Chek Aviva glucometers, Roche Diagnostics), the remainder will be centrifuged at 4000 rpm for 10 min. at 4° C., and the resulting plasma transferred into non-coated tubes and stored at −80° C. for insulin determination. Plasma insulin was measured in duplicate for each data point and animal with an HTRF insulin detection kit (62INSPEB, CisBio, USA). Plasma glucose will be measured using ACCU-CHEK Aviva glucometers (Roche Diagnostics).

### Data Evaluation and Statistics

All data was entered into Excel 2003 spreadsheets and subsequently subjected to relevant statistical analyses (GraphPad Prism, GraphPad Software, San Diego, Calif.). Results are presented as mean±SD (standard deviation) unless otherwise stated. Statistical evaluation of the data was carried out using one-way analysis of variance (ANOVA) with appropriate post-hoc analysis between control and treatment groups in cases where statistical significance is established.

### B14. Effects of Subchronic Administration of Representative Compounds of the Invention in Male Zucker Fatty Rats

### Objective

The objective of this study is to determine the subchronic effects of test compounds on body weight change, food and water consumption, as well as glucose homeostasis and insulin levels in male Zucker fatty rats up to 7 days upon oral administration. The male Zucker fatty rat was selected as an insulin resistance and genetically defined obesity model which is sensitive to the effect of different insulin sensitizers in acute as well as in chronic settings.

### Animals

Rats were individually housed in rodent cages with soft wood bedding on the bottom and equipped with water bottles. All individual cages were clearly labeled with a cage card indicating study number, group, animal number and dose level. Each animal was uniquely identified by an animal number. The animal number was designated the day the animals arrived at the animal facility. The animal room environment was controlled (targeted ranges: temperature 22±2° C.; relative humidity 50±10%; light/dark cycle: 12 hours light, 12 hours dark, lights on from 06:00 AM to 06:00 PM). A regular rodent diet (Charles River 5075 rodent chow, Purina Mills, Canada) was provided to the animals ad libitum, after food weighing. Municipal tap water was provided to the animals ad libitum via water bottles. Fresh tap water was provided after water bottle weighing.

An acclimation period of approximately 7 days for all groups was allowed between the receipt of animals and the start of treatment to accustom the rats to the laboratory environment. On experimental day −7, animals were stratified according to body weight into an appropriate number of groups of 4 or 8 animals each.

### Test Protocol

Test compound was administered, as a solution, orally, at the doses indicated. The dose volume was 5 mL/kg/day. Groups were dosed once daily around 8:00 a.m. On Day 3, on half of the animals (Subset A) and on Day 4 on the other half (Subset B), an OGTT was performed 2 hrs post-dosing (around 10:00 a.m.). The OGTT was repeated the same way on Days 7 (Subset A) and 8 (Subset B).

| | | | Total daily | Dose | Dosage | |
|---|---|---|---|---|---|---|
| Group | | Dose | dosage | Concentration | Volume | No of |
| No. | Test Article | (mg/kg) | (mg/kg) | (mg/mL) | (mL/kg/day) | Animals |
| 1 | Vehicle control | 0 | 0 | 0 | 5 | 8 |
| (Fatty) | (p.o.) | | | | | |
| 2 | Test cmpd (10 mg/kg, | 10 | 10 | 2 | 5 | 8 |
| (Fatty) | p.o.) | | | | | |
| 3 | Test cmpd (30 mg/kg, | 30 | 30 | 6 | 5 | 8 |
| (Fatty) | p.o.) | | | | | |
| 4 | Vehicle treated | 0 | 0 | 0 | 5 | 4 |
| (Lean) | (p.o.) | | | | | |

Other dose levels and concentrations can be investigated similarly.

For the study animals, the data collected from study Days −7 to 8 are reported. Body weights were recorded for all animals on Day −7 prior to initiation of dosing, at the time of group assignment and daily throughout the study period (Day 1-8). Food and water intake was measured daily throughout the study period (Day 1-8). On Day 1 (Subset A), Day 2 (Subset B), Day 3 (Subset A), Day 4 (Subset B), Day 7 (Subset A) and Day 8 (Subset B), animals from Groups 2 and 3 were sampled for blood into EDTA coated tubes (K2-EDTA microtainer tubes, Becton Dickinson) from a tail vein (˜100 μl) 2 hrs post-dose for PK analysis. Blood was centrifuged at 4000 rpm for 10 min. at 4° C., and the resulting plasma transferred into non-coated tubes and stored at −80° C. until analysis.

An oral glucose tolerance test (OGTT) was carried out in animals of all groups on Day 3 (half of the animals) as well as on day 4 (other half of the animals). This was done 2 hrs post-dose. Animals were subjected to an overnight fast (food removed the day before at 5:00 PM). Blood samples of approximately 20 μL each for plasma glucose and 230 μL for plasma insulin measurements were collected into EDTA coated tubes (K2-EDTA microtainer tubes, Becton Dickinson) from a tail vein, at 0 (pre-glucose), 15, 30, 60, and 120 min after oral administration of 1.5 g/kg glucose (dextrose, Sigma Aldrich, 450 mg/mL dosing solution). The glucose solution was administered by oral gavage via a stainless steel feeding needle (18×2″, Popper @ Sons, cat. # 20068-642, VWR). While glucose concentrations were determined from a 20 μL drop of blood (Accu-Chek Aviva glucometers, Roche Diagnostics), the remaining 230 μL was centrifuged at 4000 rpm for 10 min. at 4° C., and the resulting plasma transferred into non-coated tubes and stored a −80° C. for insulin determination. These procedures were performed on Day 7 (Subset A) and 8 (Subset B). It is worth noting that, in order to minimize blood volume withdrawal from the animals, blood samples for insulin measurement were taken only at time 0 (pre-glucose) on Day 3 and 4 and additionally at times 15, 30, 60 and 120 min. on Day 7 and 8, as stated above.

From all animals, blood was collected by a cardiac puncture on experimental Day 7 (Subset A) and 8 (Subset B) for the determination of serum concentration of free fatty acids, triglycerides, and total cholesterol. This was performed right after the OGTT. For this, 1 mL of blood was collected in pre-cooled serum separation clotting activator tubes (Sarstedt). The blood was centrifuged at 2500 rpm (4° C., 10 min), serum transferred into non-coated tubes and stored at ˜80° C. until analysis. Serum samples (250 μL each) for triglycerides, total cholesterol and free fatty acids were analyzed using appropriate methods.

Plasma insulin was measured in duplicate for each data point and animal with an HTRF insulin detection kit (62INSPEB, CisBio, USA). Plasma glucose was measured using ACCU-CHEK Aviva glucometers (Roche Diagnostics). Serum cholesterol and triglycerides was measured using standard enzyme assay kits (TGs: cat. # 11488872216, Roche Diagnostics; Chol: cat. # 11489232216, Roche Diagnostics). The measurements were performed on a Hitachi 912 analyzer. Serum free fatty acids (FFA) were measured in duplicate using a commercially available colorimetric enzyme assay kit (HR series NEFA-HR (2) kit, WAKO Chemicals). Absorbance was obtained using a GENios Pro automated plate reader (Tecan).

### Data Evaluation and Statistics

All data was entered into Excel 2003 spread sheets and subsequently subjected to relevant statistical analyses (GraphPad Prism, GraphPad Software, San Diego, Calif.). Results are presented as mean±SD (standard deviation) unless otherwise stated. Statistical evaluation of the data was carried out using one-way analysis of variance (ANOVA) with appropriate post-hoc analysis between control and treatment groups in cases where statistical significance was established.

B15. Effects of Subchronic Administration of Compounds of the Invention in Male ob/ob Mice

### Objective

The objective of this study is to determine the subchronic effects of test compounds on body weight change, food and water consumption, as well as glucose homeostasis and insulin levels in male ob/ob mice upon oral administration for up to 7 days. The male ob/ob mouse was selected as a type 2 diabetes (T2DM) and genetically defined obesity model which is sensitive to the effect of different insulin sensitizers in acute as well as in chronic settings. More precisely, this model displays a deletion in the leptin gene.

A similar study in this model was conducted to determine the acute and subchronic effects of test compounds on body weight change, food and water consumption, glucose homeostasis, insulin and glucagon levels, as well as lipid profile and brain penetration upon oral administration to the male ob/ob mice for up to 28 days.

### Animals

Mice were individually housed in rodent cages with soft wood bedding on the bottom and equipped with water bottles. All cages were clearly labeled with a cage card indicating study number, group, animal number and dose level. Each animal was uniquely identified by an animal number marked on their tail with indelible ink. The animal number was designated the day the animals arrive at the animal facility. The animal room environment was controlled (targeted ranges: temperature 22±2° C.; relative humidity 50±10%; light/dark cycle: 12 hours light, 12 hours dark, lights on from 06:00 AM to 06:00 PM). A regular rodent diet (Charles River 5075 rodent chow, Purina Mills, Canada) was provided to the animals ad libitum. Municipal tap water was provided to the animals ad libitum via water bottles. Fresh tap water was provided after water bottle weighing. An acclimation period of approximately 7 days for all groups was allowed between the receipt of animals and the start of treatment to accustom the rats to the laboratory environment. On experimental Day −7, animals were stratified according to body weight and glycemia into an appropriate number of groups of 5 or 10 animals and two groups of 5 animals.

### Test Protocol (7 Day Study)

Test compounds were administered, as a solution, orally, at the doses indicated. The dose volume will be 5 mL/kg/day. Groups were dosed once daily around 4:00 p.m. As positive controls, rosiglitazone (Avandia®), an approved anti-diabetic drug of the thiazolidinediones family (ppar gamma agonist) which has been specifically reported to normalize glycemia in the ob/ob mouse model (Liu et al., J. Med. Chem.; 46: 2093-2103, 2003) was used. The CB1 receptor antagonist rimonabant (Accomplia®) was reported to reduce body weight and food intake in different models of Type 2 diabetes and obesity and was also employed (Rasmussen and Huskinson Behavioral Pharmacol. 2008, 19, 735-742,; Bobo, G.; et al. Hepathology 2007, 46, 122-129; Di Marzo; et al., Nature 2001, 410, 822-825).

| | | | Total | Dose | Dosage | |
|---|---|---|---|---|---|---|
| Group | | Dose | daily dose | Concentration | Volume | No of |
| No. | Test Article | (mg/kg) | (mg/kg) | (mg/mL) | (mL/kg/day) | Animals |
| 1 | Vehicle | 0 | 0 | 0 | 5 | 10 |
| (ob/ob) | control (p.o.) | | | | | |
| 2 | Test cmpd | 10 | 10 | 2 | 5 | 10 |
| (ob/ob) | (10 mg/kg, | | | | | |
| | p.o.) | | | | | |
| 3 | Test cmpd | 30 | 30 | 6 | 5 | 10 |
| (ob/ob) | (30 mg/kg, | | | | | |
| | p.o.) | | | | | |
| 4 | Test cmpd | 100 | 100 | 20 | 5 | 10 |
| (ob/ob) | (100 mg/kg, | | | | | |
| | p.o.) | | | | | |
| 5 | Rosiglitazone | 3 | 3 | 0.6 | 5 | 5 |
| (ob/ob) | (3 mg/kg, | | | | | |
| | p.o.) | | | | | |
| 6 | Rimonabant | 10 | 10 | 2 | 5 | 5 |
| (ob/ob) | (10 mg/kg, | | | | | |
| | p.o.) | | | | | |
| 7 | Vehicle | 0 | 0 | 0 | 5 | 5 |
| (Lean) | treated (p.o.) | | | | | |

Other dose levels and concentrations can be investigated similarly.

For, the study animals, the data collected from study Day −7 to Day 8 were reported. Body weights were recorded for all animals on Day −7 prior to initiation of dosing, at the time of group assignment and daily throughout the study period (Day 1-8). Food and water intake was Measured 4 hrs post-dosing, 2 hrs after the beginning of the dark cycle (around 8:00 p.m.) on Day 1 and 7 (Subset A) as well as on Day 2 and 8 (Subset B) and then daily in 24 h intervals from Day 3 through Day 8. On Day 1 (Subset A) and Day 2 (Subset B), blood was sampled from 3 animals/group from Groups 2 through 4 into EDTA coated tubes (K2-EDTA microtainer tubes, Becton Dickinson) from a tail vein (˜100 μL) 4 hrs post-dose for PK analysis. Blood was centrifuged at 4000 rpm for 10 min. at 4° C., and the resulting plasma transferred into non-coated tubes and stored at −80° C. until analysis. The same procedures were repeated on Day 7 (Subset A) and Day 8 (Subset B) 24 hrs post-dose. From all animals, a terminal blood sample was collected (approximately 5 mL total) by cardiac puncture on experimental Day 7 (Subset A) and 8 (Subset B) for the determination of plasma concentrations of glucose and insulin and serum concentrations of free fatty acids, triglycerides and total cholesterol. Blood samples for plasma insulin measurements (250 μL) were collected into EDTA coated tubes (K2-EDTA microtainer tubes, Becton Dickinson). Blood was centrifuged at 4000 rpm for 10 min. at 4° C., and the resulting plasma transferred into non-coated tubes and stored at −80° C. until analysis. Additionally, 1 mL of blood was collected in pre-cooled serum separation clotting activator tubes (Sarstedt). The blood was centrifuged at 2500 rpm (4° C., 10 min), serum transferred into non-coated tubes and stored at −80° C. until analysis. Serum samples (250 μL each) for triglycerides, total cholesterol and free fatty acids were analyzed using appropriate methods.

Animals from Groups 1-4 had their brain removed immediately after the terminal bleed for test compound brain concentration measurement. Brains were kept on ice and put at −80° C. until analysis.

Plasma insulin was measured in duplicate for each data point and animal with an HTRF insulin detection kit (62INSPEB, CisBio, USA). Plasma glucose (20 μL blood sample) was measured using ACCU-CHEK Aviva glucometers (Roche Diagnostics). Serum cholesterol and triglycerides were measured using standard enzyme assay kits (TGs: cat. # 11488872216, Roche Diagnostics; Chol: cat. # 11489232216, Roche Diagnostics) on a Hitachi 912 analyzer. Serum free fatty acids (FFA) were measured in duplicate using a commercially available colorimetric enzyme assay kit (HR series NEFA-HR (2) kit, WAKO Chemicals). Absorbance was read on a GENios Pro automated plate reader (Tecan).

### Test Protocol (15 Day Study)

Test compounds were administered, as a solution, orally, at the doses indicated. The dose volume was 5 mlJkg/day. Groups 1-4 (Subset A) were especially dosed at 9:00 a.m. on Day 1, Day 7, Day 14 and Day 15. Otherwise, these groups were dosed once daily around 3:00 p.m. from Day 2 through Day 6 and from day 8 through 13. Groups 5-8 (Subset B) were dosed once daily around 3:00 p.m. from Day 1 through Day 14 and then at 9:00 a.m. on Day 15.

| | | | Total | Dose | Dosage | |
|---|---|---|---|---|---|---|
| Group | | Dose | daily dose | Concentration | Volume | No of |
| No. | Test Article | (mg/kg) | (mg/kg) | (mg/mL) | (mL/kg/day) | Animals |
| Subset A | | | | | | |
| 1 (ob/ob) | Vehicle | 0 | 0 | 0 | 5 | 6 |
| | control (p.o.) | | | | | |
| 2 (ob/ob) | Test cmpd 1 | 10 | 10 | 2 | 5 | 6 |
| | (10 mg/kg, | | | | | |
| | p.o.) | | | | | |
| 3 (ob/ob) | Test cmpd 1 | 50 | 50 | 10 | 5 | 6 |
| | (50 mg/kg, | | | | | |
| | p.o.) | | | | | |
| 4 (Lean) | Vehicle | 0 | 0 | 0 | 5 | 6 |
| | control (p.o.) | | | | | |
| Subset B | | | | | | |
| 5 (ob/ob) | Vehicle | 0 | 0 | 0 | 5 | 6 |
| | control (p.o.) | | | | | |
| 6 (ob/ob) | Test cmpd 2 | 10 | 10 | 2 | 5 | 6 |
| | (10 mg/kg, | | | | | |
| | p.o.) | | | | | |
| 7 (ob/ob) | Test cmpd 2 | 50 | 50 | 10 | 5 | 6 |
| | (50 mg/kg, | | | | | |
| | p.o.) | | | | | |
| 8 (Lean) | Vehicle | 0 | 0 | 0 | 5 | 6 |
| | control (p.o.) | | | | | |

Other dose levels and concentrations can be investigated similarly.

For the study animals, the data collected from study Day −7 to Day 15 were reported. Body weights were recorded for all animals on Day −7 prior to initiation of dosing, at the time of group assignment and daily throughout the study period (Day 1-15). Fasting glucose levels from Groups 1-4 (subset A) were monitored on day 1, 7 and 14. Non-fasting glucose levels from Groups 5-8 (Subset B) were monitored on Day 1, 7 and 14. Food and water intake were measured acutely 20 min, 1 hr, 2 hr and 4 hr post-dose in one subset of animals (Groups 1-4, Subset A) on Day 1 as well as on Day 7 and daily in 24 h intervals from Day I through Day 14 in Subset B animals (Groups 5-8). On Day 14, in all animals from Groups 1-4 (Subset A) an oral glucose tolerance test OGTT) was performed. For this, the animals were fasted overnight. Blood samples for plasma glucose concentrations were taken at 0 (pre-glucose), 15, 30, 60 and 120 min after oral administration of 1.5 g/kg glucose (dextrose, Sigma Aldrich, 450 mg/ml dosing solution). The glucose solution was administered by oral gavage via a stainless steel feeding needle (18×2″, Popper @ Sons, cat. # 20068-642, VWR). Glucose concentrations were determined from a 20 μL drop of blood and measurements performed on an Accu-Chek Aviva glucometer (Roche Diagnostics).

On Day 15, blood was sampled from all animals of Groups 2 and 3 (Subset A) into EDTA coated tubes (K2-EDTA microtainer tubes, Becton Dickinson) from a tail vein (˜100 μl) 0, 15 min, 30 min, 1 hr, 2 hr, and 4 hr post-dose for PK analysis (n=2 mice/treatment group/time point). Blood was centrifuged at 4000 rpm for 10 min at 4° C., and the resulting plasma transferred into non-coated tubes and stored at −80° C. until analysis. From all animals of Groups 5-8 (Subset B), a terminal blood sample was collected (approximately 1 mL total) by cardiac puncture on experimental Day 15 for the determination of plasma concentrations of insulin, glucagon, free fatty acids, triglycerides, total cholesterol, LDL, HDL as well as HDL/total cholesterol ratio. Blood samples were collected into EDTA coated tubes (K2-EDTA microtainer tubes, Becton Dickinson). Blood was centrifuged at 4000 rpm for 10 min at 4° C., and the resulting plasma transferred into non-coated tubes and stored at −80° C. until analysis.

On Day 15, animals from Groups 1-3 (Subset A) as well as from Groups 6 and 7 (Subset B) had their brain removed 30 min, 1 hr, 2 hr or 4 hr post-dose for test compound brain concentration measurement (n=3 mice/treatment group/time point). Brains were kept on ice and frozen at −80° C. until analysis.

Plasma insulin and glucagon were measured for each data point and animal with an HTRF insulin detection kit (62INSPEB, CisBio, USA). Plasma glucose (20 μL blood sample) will be measured using an ACCU-CHEK Aviva glucometer (Roche Diagnostics). For clinical chemistry determinations, 35 μL of plasma was analysed on a Cholestech LDX analyzer (ManthaMed, Mississauga, ON, Canada) for triglycerides, HDL cholesterol, non-HDL cholesterol, LDL cholesterol, total cholesterol (TC) and TC/HDL ratio. Serum free fatty acids (FFA) were measured in duplicate using a commercially available colorimetric enzyme assay kit (HR series NEFA-HR (2) kit, WAKO Chemicals). Absorbance was read on a GENios Pro automated plate reader (Tecan).

### Test Protocol (28 Day Study)

Test compounds were administered, as a solution, orally, at the doses indicated. The dose volume was 5 mL/kg/day. Groups 1-4 (Subset A) were especially dosed at 9:00 a.m. on Day 1, Day 7, Day 14, Day 21 and Day 28. Otherwise, these groups were dosed once daily around 3:00 p.m. from Day 2 through Day 6, from day 8 through 13, from Day 15 through Day 20 and from Day 22 through 28. Groups 5-8 (Subset B) were dosed once daily around 3:00 p.m. from Day 1 through Day 27 and then at 9:00 a.m. on Day 28.

| | | | Total | | | |
|---|---|---|---|---|---|---|
| | | | daily | Dose | Dosage | |
| Group | | Dose | dose | Concentration | Volume | No of |
| No. | Test Article | (mg/kg) | (mg/kg) | (mg/mL) | (mL/kg/day) | Animals |
| Subset A | | | | | | |
| 1 (ob/ob) | Vehicle | 0 | 0 | 0 | 5 | 8 |
| | control (p.o.) | | | | | |
| 2 (ob/ob) | Test cmpd 1 | 15 | 15 | 3 | 5 | 8 |
| | (15 mg/kg, p.o.) | | | | | |
| 3 (ob/ob) | Test cmpd 1 | 75 | 75 | 15 | 5 | 8 |
| | (75 mg/kg, | | | | | |
| | p.o.) | | | | | |
| 4 (Lean) | Vehicle | 0 | 0 | 0 | 5 | 8 |
| | control (p.o.) | | | | | |
| Subset B | | | | | | |
| 5 (ob/ob) | Vehicle | 0 | 0 | 0 | 5 | 7 |
| | control (p.o.) | | | | | |
| 6 (ob/ob) | Test cmpd 2 | 15 | 15 | 3 | 5 | 7 |
| | (15 mg/kg, p.o.) | | | | | |
| 7 (ob/ob) | Test cmpd 2 | 75 | 75 | 15 | 5 | 7 |
| | (75 mg/kg, | | | | | |
| | p.o.) | | | | | |
| 8 (Lean) | Vehicle | 0 | 0 | 0 | 5 | 6 |
| | control (p.o.) | | | | | |

Other dose levels and concentrations can be investigated similarly.

For the study animals, the data collected from study Day −7 to Day 28 were reported. Body weights were recorded for all animals on Day −7 prior to initiation of dosing, at the time of group assignment and daily throughout the study period (Day 1-28). Fasting (16 hr fast) glucose levels from Groups 1-4 (subset A) were monitored on day 1, 7, 14, 21 and 28. Non-fasting glucose levels from Groups 5-8 (Subset B) were monitored on Day 1, 7, 14, 21 and 28. Food and water intake were measured acutely 20 min, 1 hr, 2 hr and 4 hr post-dose in one subset of animals (Groups 1-4, Subset A) on Day 1, Day 7 as well as on Day 21 and daily in 24 h intervals from Day 1 through Day 28 in Subset B animals (Groups 5-8). On Day 1 and Day 14, in all animals from Groups 1-4 (Subset A) an oral glucose tolerance test OGTT) was performed. For this, the animals were fasted overnight. Blood samples for plasma glucose concentrations were taken at 0 (pre-glucose), 15, 30, 60 and 120 min after oral administration of 1.5 g/kg glucose (dextrose, Sigma Aldrich, 450 mg/mL dosing solution). The glucose solution was administered by oral gavage via a stainless steel feeding needle (18×2″, Popper @ Sons, cat. # 20068-642, VWR). Glucose concentrations were determined from a 20 μL drop of blood and measurements performed on an Accu-Chek Aviva glucometer (Roche Diagnostics).

On Day 28/29, blood was sampled from all animals of Groups 2 and 3 (Subset A) into EDTA coated tubes (K₂-EDTA microtainer tubes, Becton Dickinson) from a tail vein (˜100 μl) 0, min, 30 min, 1 hr, 2 hr, and 4 hr post-dose for PK analysis (n=2 mice/treatment group/time point). Blood was centrifuged at 4000 rpm for 10 min at 4° C., and the resulting plasma transferred into non-coated tubes and stored at −80° C. until analysis. A terminal blood sample was collected (approximately 1 mL total) from Groups 2 and 3 (Subset A) and Groups 5-8 (Subset B) by cardiac puncture on experimental Day 28/29 for the determination of plasma concentrations of insulin, glucagon, acylated and unacylated ghrelin, growth hormone, GLP-1, IGF-1, free fatty acids, triglycerides and total cholesterol. Blood samples were collected into EDTA coated tubes (K₂-EDTA microtainer tubes, Becton Dickinson). Blood was centrifuged at 4000 rpm for 10 min at 4° C., and the resulting plasma transferred into non-coated tubes and stored at −80° C. until analysis.

On Day 28/29, animals from Groups 1-3 (Subset A) as well as from Groups 6 and 7 (Subset B) had their brains removed 30 min, 1 hr, 2 hrs or 4 hrs post-dose for test compound brain concentration measurement (n=3 mice/treatment group/time point). Brains were kept on ice and frozen at −80° C. until analysis.

On Day 28/29, all animals from Groups 1-4 (Subset A) as well as from Groups 5-8 (Subset B) had their liver removed after the terminal bleed for determination of free fatty acids, triglycerides and total cholesterol levels. Livers were kept on ice and frozen at −80° C. until analysis.

Plasma insulin and glucagon were measured for each data point and animal with an HTRF insulin detection kit (62INSPEB, CisBio, USA). Plasma glucose (20 μL blood sample) will be measured using an ACCU-CHEK Aviva glucometer (Roche Diagnostics). Plasma acylated and unacylated ghrelin as well as growth hormone were measured using enzyme immunoassay kits (A05117, A05118 and A05104, respectively, from Alpco Diagnostics, USA). Plasma IGF-1 and GLP-1 were measured using IGF-1 (mouse, rat) ELISA and GLP-1 (ac-tive 7-36) ELISA kits from Alpco Diagnostics (USA). For clinical chemistry determinations, 35 μL of plasma was analysed on a Cholestech LDX analyzer (ManthaMed, Mississauga, ON, Canada) for triglycerides and serum cholesterol. Serum free fatty acids (FFA) were measured in duplicate using a commercially available colorimetric enzyme assay kit (HR series NEFA-HR (2) kit, WAKO Chemicals). Absorbance was read on a GENios Pro automated plate reader (Tecan). Liver free fatty scids, triglycerides and total cholesterol levels were measured Using commercially available colorimetric enzyme assay kits (free fatty acid quantification kit K612-100, triglyceride quantification kit K622-100 and cholesterol/cholesteryl ester quantitation kit K603-100, Biovision, Mountain View, Calif., USA).

Data Evaluation and Statistics

All data was entered into Excel 2003 or 2007 spreadsheets and subsequently subjected to relevant statistical analyses (GraphPad Prism or GraphPad Instat, GraphPad Software, San Diego, Calif.). Results are presented as mean±SD (standard deviation) unless otherwise stated. Statistical evaluation of the data is carried out using one-way analysis of variance (ANOVA) with appropriate post-hoc analysis between control and treatment groups in cases where statistical significance was established.

B16. hERG Channel Inhibition

The product of the hERG (human ether-a-go-go) gene is an ion channel responsible for the I_{Kr} repolarizing current, where alterations to this current have been shown to elongate the cardiac action potential and promote the appearance of early after-depolarizations. Direct interactions of compounds with the hERG channel account for the majority of known cases of cardiotoxicity.

### Method

The key aspects of the experimental method are as follows:
hERG gene stably expressed in HEK293 cells
Borosilicate microelectrodes are used to record whole cell I_{Kr} currents over a predetermined pulse protocol
Control currents are recorded in the absence of inhibitor (E-4031, positive control) or test compound.
Compounds are tested at 1 and 10 μM:
   The compound is allowed to perfuse the cells for 5 min.
   Three currents are then recorded by applying the same pulse protocol as in control conditions.
A single concentration (0.5 μM) of a positive control (for example, E-4031, known inhibitor of I_{Kr}) is also tested

### Results

Compounds 1712, 1848 and 1929 showed no significant effect on hERG channel function in comparison to vehicle (0.1% DMSO) controls up to 100 μM.

### 5. Pharmaceutical Compositions

The macrocyclid compounds of the present invention or pharmacologically acceptable salts thereof according to the invention may be formulated into pharmaceutical compositions of various dosage forms. To prepare the pharmaceutical compositions of the invention, one or more compounds, including optical isomers, enantiomers, diastereomers, racemates or stereochemical mixtures thereof, or pharmaceutically acceptable salts thereof as the active ingredient is intimately mixed with appropriate carriers and additives according to techniques known to those skilled in the art of pharmaceutical formulations.

A pharmaceutically acceptable salt refers to a salt form of the compounds of the present invention in order to permit their use or formulation as pharmaceuticals and which retains the biological effectiveness of the free acids and bases of the specified compound and that is not biologically or otherwise undesirable. Examples of such salts are described in Handbook of Pharmaceutical Salts: Properties, Selection, and Use, Wermuth, C. G. and Stahl, P. H. (eds.), Wiley-Verlag Helvetica Acta, Zurich, 2002 [ISBN 3-906390-26-8]. Examples of such salts include alkali metal salts and addition salts of free acids and bases. Examples of pharmaceutically acceptable salts, without limitation, include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, xylenesulfonates, phenylacetates, phenylpropionates, phenylbutyrates, citrates, lactates, γ-hydroxybutyrates, glycollates, tartrates, methanesulfonates, ethane sulfonates, propanesulfonates, toluenesulfonates, naphthalene-1-sulfonates, naphthalene-2-sulfonates, and mandelates.

If an inventive compound is a base, a desired salt may be prepared by any suitable method known to those skilled in the art, including treatment of the free base with an inorganic acid, such as, without limitation, hydrochloric acid, hydrobromic acid, hydroiodic, carbonic acid, sulfuric acid, nitric acid, phosphoric acid, and the like, or with an organic acid, including, without limitation, formic acid, acetic acid, propionic acid, maleic acid, succinic acid, mandelic acid, fumaric acid, malonic acid, pyruvic acid, oxalic acid, stearic acid, ascorbic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid, 2-hydroxyethanesulfonic acid, benzenesulfonic acid, cyclohexylaminosulfonic acid or the like.

If an inventive compound is an acid, a desired salt may be prepared by any suitable method known to the art, including treatment of the free acid with an inorganic or organic base, such as an amine (primary, secondary, or tertiary); an alkali metal or alkaline earth metal hydroxide; or the like. Illustrative examples of suitable salts include organic salts derived from amino acids such as glycine, lysine and arginine; ammonia; primary, secondary, and tertiary amines such as ethylenediamine, N,N′-dibenzylethylenediamine, diethanolamine, choline, and procaine, and cyclic amines, such as piperidine, morpholine, and piperazine; as well as inorganic salts derived from sodium, calcium, potassium, magnesium, manganese, iron, copper, zinc, aluminum, and lithium.

The carriers and additives used for such pharmaceutical compositions can take a variety of forms depending on the anticipated mode of administration. Thus, compositions for oral administration may be, for example, solid preparations such as tablets, sugar-coated tablets, hard capsules, soft capsules, granules, powders and the like, with suitable carriers and additives being starches, sugars, binders, diluents, granulating agents, lubricants, disintegrating agents and the like. Because of their ease of use and higher patient compliance, tablets and capsules represent the most advantageous oral dosage forms for many medical conditions.

Similarly, compositions for liquid preparations include solutions, emulsions, dispersions, suspensions, syrups, elixirs, and the like with suitable carriers and additives being water, alcohols, oils, glycols, preservatives, flavoring agents, coloring agents, suspending agents, and the like. Typical preparations for parenteral administration comprise the active ingredient with a carrier such as sterile water or parenterally acceptable oil including polyethylene glycol, polyvinyl pyrrolidone, lecithin, arachis oil or sesame oil, with other additives for aiding solubility or preservation may also be included. In the case of a solution, it can be lyophilized to a powder and then reconstituted immediately prior to use. For dispersions and suspensions, appropriate carriers and additives include aqueous gums, celluloses, silicates or oils.

The pharmaceutical compositions according to embodiments of the present invention include those suitable for oral, rectal, topical, inhalation (e.g., via an aerosol) buccal (e.g., sub-lingual), vaginal, topical (i.e., both skin and mucosal surfaces, including airway surfaces), transdermal administration and parenteral (e.g., subcutaneous, intramuscular, intradermal, intraarticular, intrapleural, intraperitoneal, intrathecal, intracerebral, intracranially, intraarterial, or intravenous), although the most suitable route in any given case will depend on the nature and severity of the condition being treated and on the nature of the particular active agent which is being used.

Compositions for injection will include the active ingredient together with suitable carriers including propylene glycol-alcohol-water, isotonic water, sterile water for injection (USP), emulPhor™-alcohol-water, cremophor-EL™ or other suitable carriers known to those skilled in the art. These carriers may be used alone or in combination with other conventional solubilizing agents such as ethanol, propylene glycol, or other agents known to those skilled in the art.

Where the macrocyclic compounds of the present invention are to be applied in the form of solutions or injections, the compounds may be used by dissolving or suspending in any conventional diluent. The diluents may include, for example, physiological saline, Ringer's solution, an aqueous glucose solution, an aqueous dextrose solution, an alcohol, a fatty acid ester, glycerol, a glycol, an oil derived from plant or animal sources, a paraffin and the like. These preparations may be prepared according to any conventional method known to those skilled in the art.

Compositions for nasal administration may be formulated as aerosols, drops, powders and gels. Aerosol formulations typically comprise a solution or fine suspension of the active ingredient in a physiologically acceptable aqueous or non-aqueous solvent. Such formulations are typically presented in single or multidose quantities in a sterile form in a sealed container. The sealed container can be a cartridge or refill for use with an atomizing device. Alternatively, the sealed container may be a unitary dispensing device such as a single use nasal inhaler, pump atomizer or an aerosol dispenser fitted with a metering valve set to deliver a therapeutically effective amount, which is intended for disposal once the contents have been completely used. When the dosage form comprises an aerosol dispenser, it will contain a propellant such as a compressed gas, air as an example, or an organic propellant including a fluorochlorbhydrocarbon or fluorohydrocarbon.

Compositions suitable for buccal or sublingual administration include tablets, lozenges and pastilles, wherein the active ingredient is formulated with a carrier such as sugar and acacia, tragacanth or gelatin and glycerin.

Compositions for rectal administration include suppositories containing conventional suppository base such as cocoa butter.

Compositions suitable for transdermal administration include ointments, gels and patches.

Other compositions known to those skilled in the art can also be applied for percutaneous or subcutaneous administration, such as plasters.

Further, in preparing such pharmaceutical compositions comprising the active ingredient or ingredients in admixture with components necessary for the formulation of the compositions, other conventional pharmacologically acceptable additives may be incorporated, for example, excipients, stabilizers, antiseptics, wetting agents, emulsifying agents, lubricants, sweetening agents, coloring agents, flavoring agents, isotonicity agents, buffering agents, antioxidants and the like. As the additives, there may be mentioned, for example, starch, sucrose, fructose, lactose, glucose, dextrose, mannitol, sorbitol, precipitated calcium carbonate, crystalline cellulose, carboxymethylcellulose, dextrin, gelatin, acacia, EDTA, magnesium stearate, talc, hydroxypropylmethylcellulose, sodium metabisulfite, and the like.

In some embodiments, the composition is provided in a unit dosage form such as a tablet or capsule.

In further embodiments, the present invention provides kits including one or more containers comprising pharmaceutical dosage units comprising an effective amount of one or more compounds of the present invention.

The present invention further provides prodrugs comprising the compounds described herein. The term “prodrug” is intended to mean a compound that is converted under physiological conditions or by solvolysis or metabolically to a specified compound that is pharmaceutically active. The “prodrug” can be a compound of the present invention that has been chemically derivatized such that, (i) it retains some, all or none of the bioactivity of its parent drug compound, and (ii) it is metabolized in a subject to yield the parent drug compound. The prodrug of the present invention may also be a “partial prodrug” in that the compound has been chemically derivatized such that, (i) it retains some, all or none of the bioactivity of its parent drug compound, and (ii) it is metabolized in a subject to yield a biologically active derivative of the compound. Known techniques for derivatizing compounds to provide prodrugs can be employed. Such methods may utilize formation of a hydrolyzable coupling to the compound.

The present invention further provides that the compounds of the present invention may be administered in combination with a therapeutic agent used to prevent and/or treat metabolic and/or endocrine disorders, obesity and obesity-associated disorders, appetite or eating disorders, addictive disorders, cardiovascular disorders, gastrointestinal disorders, genetic disorders, hyperproliferative disorders and inflammatory disorders. Exemplary agents include analgesics including opioid analgesics, anesthetics, antifungals, antibiotics, antiinflammatories, including nonsteroidal anti-inflammatory agents, anthelmintics, antiemetics, antihistamines, antihypertensives, antipsychotics, antiarthritics, antitussives, antivirals, cardioactive drugs, cathartics, chemotherapeutic agents such as DNA-interactive agents, antimetabolites, tubulin-interactive agents, hormonal agents, and agents such as asparaginase or hydroxyurea, corticoids (steroids), antidepressants, depressants, diuretics, hypnotics, minerals, nutritional supplements, parasympathomimetics, hormones such as corticotrophin releasing hormone, adrenocorticotropin, growth hormone releasing hormone, growth hormone, thyrptropin-releasing hormone and thyroid stimulating hormone, sedatives, sulfonamides, stimulants, sympathomimetics, tranquilizers, vasoconstrictors, vasodilators, vitamins and xanthine derivatives.

Other therapeutic agents that can be used in combination with the compounds of the present invention include a GLP-1 agonist, a DPP-IV inhibitor, an amylin agonist, a PPAR-α agonist, a PPAR-γ agonist, a PPAR-α/γ dual agonist, a GDIR or GPR119 agonist, a PTP-1B inhibitor, a peptide YY agonist, an 11β-hydroxysteroid dehydrogenase (11β-HSD)-1 inhibitor, a sodium-dependent renal glucose transporter type 2 (SGLT-2) inhibitor, a glucagon antagonist, a glucokinase activator, an α-glucosidase inhibitor, a glucocorticoid antagonist, a glycogen synthase kinase 3β (GSK-3β) inhibitor, a glycogen phosphorylase inhibitor, an AMP-activated protein kinase (AMPK) activator, a fructose-1,6-biphosphatase inhibitor, a sulfonyl urea receptor antagonist, a retinoid X receptor activator, a 5-HT₁ₐ agonist, a 5-HT_{2c} agonist, a 5-HT₆ antagonist, a cannabioid antagonist or inverse agonist, a melanin concentrating hormone-1 (MCH-1) antagonist, a melanocortin-4 (MC4) agonist, a leptin agonist, a retinoic acid receptor agonist, a stearoyl-CoA desaturase-1 (SCD-1) inhibitor, a neuropeptide Y Y2 receptor agonist, a neuropeptide Y Y4 receptor agonist, a neuropeptide Y Y5 receptor antagonist, a neuronal nicotinic receptor α₄β₂ agonist a diacylglycerol acyltransferase 1 (DGAT-1) inhibitor, a thyroid receptor agonist, a lipase inhibitor, a fatty acid synthase inhibitor, a glycerol-3-phosphate acyltransferase inhibitor, a CPT-1 stimulant, an α_{1A}-adrenergic receptor agonist, an α_{2A}-adrenergic receptor agonist, a β₃-adrenergic receptor agonist, a histamine H3 receptor antagonist, a cholecystokinin A receptor agonit and a GABA-A agonist.

Subjects suitable to be treated according to the present invention include, but are not limited to, avian and mammalian subjects, and are preferably mammalian. Mammals of the present invention include, but are not limited to, canines, felines, bovines, caprines, equines, ovines, porcines, rodents (e.g. rats and mice), lagomorphs, primates, humans, and the like, and mammals in utero. Any mammalian subject in need of being treated according to the present invention is suitable. Human subjects are preferred. Human subjects of both genders and at any stage of development (i.e., neonate, infant, juvenile, adolescent, adult) can be treated according to the present invention.

Illustrative avians according to the present invention include chickens, ducks, turkeys, geese, quail, pheasant, ratites (e.g., ostrich) and domesticated birds (e.g., parrots and canaries), and birds in ovo.

The present invention is primarily concerned with the treatment of human subjects, but the invention can also be carried out on animal subjects, particularly mammalian subjects such as mice, rats, dogs, cats, livestock and horses for veterinary purposes, and for drug screening and drug development purposes.

In therapeutic use for treatment of conditions in mammals (i.e. humans or animals) for which an antagonist or inverse agonist of the ghrelin receptor is effective, the compounds of the present invention or an appropriate pharmaceutical composition thereof may be administered in an effective amount. Since the activity of the compounds and the degree of the therapeutic effect vary, the actual dosage administered will be determined based upon generally recognized factors such as age, condition of the subject, route of delivery and body weight of the subject. The dosage will be from about 0.1 to about 100 mg/kg, administered orally 1-4 times per day. In addition, compounds may be administered by injection at approximately 0.01-20 mg/kg per dose, with administration 1-4 times per day. Treatment could continue for weeks, months or longer. Determination of optimal dosages for a particular situation is within the capabilities of those skilled in the art.

### 6. Methods of Use

The compounds of the present invention can be used for the prevention and treatment of a range of medical conditions including, but not limited to, metabolic and/or endocrine disorders, obesity and obesity-associated disorders, appetite or eating disorders, addictive disorders, cardiovascular disorders, gastrointestinal disorders, genetic disorders, hyperproliferative disorders, central nervous system disorders, inflammatory disorders and combinations thereof where the disorder may be the result of multiple underlying maladies.

Metabolic and/or endocrine disorders include, but are not limited to, obesity, diabetes, in particular, type II diabetes, metabolic syndrome, non-alcoholic fatty liver disease (NAFLD), non-alcoholic steatohepatitis (NASH) and steatosis. Obesity and obesity-associated disorders include, but are not limited to, retinopathy, hyperphagia and disorders involving regulation of food intake and appetite control in addition to obesity being characterized as a metabolic and/or endocrine disorder. Appetite or eating disorders include, but are not limited to, Prader-Willi syndrome and hyperphagia. Addictive disorders include, but are not limited to, alcohol dependence or abuse, illegal drug dependence or abuse, prescription drug dependence or abuse and chemical dependence or abuse (non-limiting examples include alcoholism, narcotic addiction, stimulant addiction, depressant addiction and nicotine addiction). Cardiovascular disorders include, but are not limited to, hypertension and dyslipidemia. Gastrointestinal disorders include, but are not limited to, irritable bowel syndrome, dyspepsia, opioid-induced bowel dysfunction and gastroparesis. Hyperproliferative disorders include, but are not limited to, tumors, cancers, and neoplastic tissue, which further include disorders such as breast cancers, osteosarcomas, angiosarcomas, fibrosarcomas and other sarcomas; leukemias, lymphomas, sinus tumors, ovarian, uretal, bladder, prostate and other genitourinary cancers, colon, esophageal and stomach cancers and other gastrointestinal cancers, lung cancers, myelomas, pancreatic cancers, liver cancers, kidney cancers, endocrine cancers, skin cancers, and brain or central and peripheral nervous (CNS) system tumors, malignant or benign, including gliomas and neuroblastomas. Central nervous system disorders include, but are not limited to, seizures, seizure disorders, epilepsy, status epilepticus, migraine headache, cortical spreading depression, headache, intracranial hypertension, central nervous system edema, neuropsychiatric disorders, neurotoxicity, head trauma, stroke, ischemia, hypoxia, anxiety, depression, Alzheimer's Disease, obesity, Parkinson's Disease, smoking cessation, additive disorders such as alcohol addiction, addiction to narcotics (such as cocaine addiction, heroin addiction, opiate addiction, etc.), anxiety and neuroprotection (e.g. reducing damage following stroke, reducing damage from neurodegenerative diseases like Alzheimer's, protecting against toxicity damage from ethanol. Inflammatory disorders include, but are not limited to, general inflammation, arthritis, for example, rheumatoid arthritis and osteoarthritis, and inflammatory bowel disease. The compounds of the present invention can further be used to prevent and/or treat cirrhosis and chronic liver disease. As used herein, “treatment” is not necessarily meant to imply cure or complete abolition of the disorder or symptoms associated therewith.

The compounds of the present invention can further be utilized for the preparation of a medicament for the treatment of a range of medical conditions including, but not limited to, metabolic and/or endocrine disorders, obesity and obesity-associated disorders, appetite or eating disorders, cardiovascular disorders, gastrointestinal disorders, genetic disorders, hyperproliferative disorders and inflammatory disorders.

Further embodiments of the present invention will now be described with reference to the following examples. It should be appreciated that these examples are for the purposes of illustrating embodiments of the present invention, and do not limit the scope of the invention.

### Example 1

### Amino Acid Building Blocks

### Example AA1

### Standard Procedure for the Synthesis of H-(3Me)Cpg-OH

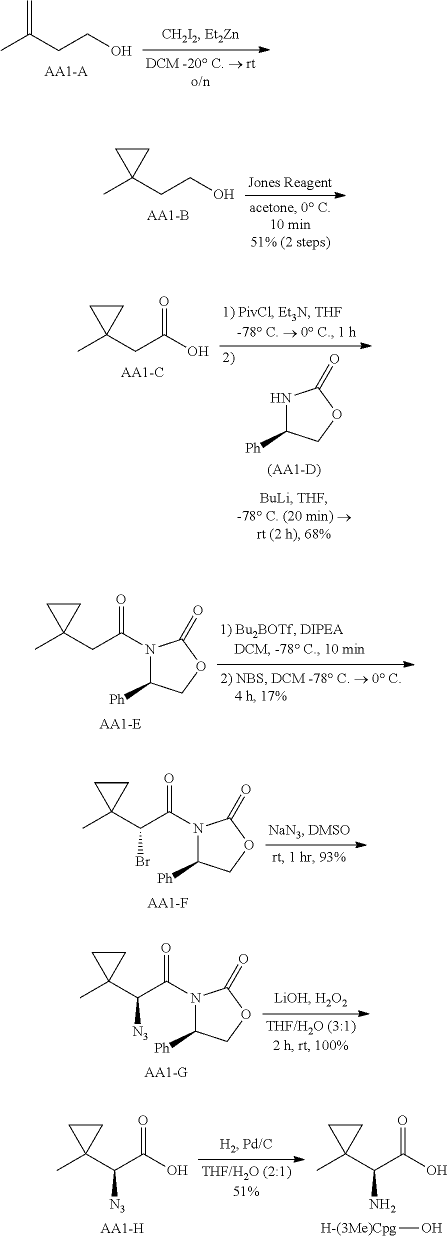

Step AA1-1: Cyclopropanation. To a solution of 3-methyl-3-buten-1-ol (AA1-A, 3.52 mL, 34.8 mmol, 1.0 eq) in DCM (350 mL) at −20° C. under an argon atmosphere, was carefully added neat diethylzinc (17.9 mL, 174 mmol, 5.0 eq) and diiodomethane (28.1 mL, 348 mmol, 10.0 eq) and the temperature quickly raised to 0° C. (CAUTION: Temperature control is very important. Diiodomethane (mp: 5-8° C.) and diethylzinc (mp: −28° C.) can freeze and stop agitation suddenly with a risk of explosion upon melting). The reaction was warmed slowly to room temperature and stirred overnight. To the mixture was added saturated NH₄Cl (aq) and the aqueous phase extracted with Et₂O (3×). The combined organic phase was washed with saturated aq. NaHCO₃ (2×), brine (1×), dried over MgSO₄, filtered, then the filtrate concentrated by a rotary evaporator under low temperature and pressure due to the low boiling point of the product to afford 2-(1-methylcyclopropyl)ethanol (AA1-B, 12.4 g, >100%, orange liquid), which was used without further purification in the next step.

Step AA1-2. Oxidation. A solution of AA1-B (34.8 mmol, 1.0 eq) in acetone (350 mL) was cooled at 0° C. Jones reagent was added until the solution remained orange in color and stirred for an additional 10 min at 0° C. Water was added and the resulting aqueous phase extracted with Et₂O (3×). Then the combined organic phase was extracted with 1M sodium carbonate (3×). The combined aqueous phase was washed with Et₂O (3×), then acidified to pH=2 with 6N HCl at 0° C. and extracted with Et₂O (3×). The combined organic phase was washed with water (1×), brine (1×), dried over MgSO₄, filtered, then the filtrate concentrated in vacuo to yield 2-(1-methylcyclopropyl)acetic acid (AA1-C, 2.03 g, 51% for 2 steps) as a colorless liquid with an obnoxious odor.

Step AA1-3. Chiral auxiliary anchoring. To AA1-C (2.03 g, 17.8 mmol, 1.0 eq) in THF (200 mL) at −78° C., was added Et₃N (2.98 mL, 21.4 mmol, 1.2 eq) and PivCl (2.41 mL, 19.6 mmol, 1.1 eq) to form a mixed anhydride. This mixture was stirred 15 min at −78° C. and 45 min at 0° C., then cooled down to −78° C. Separately, to the chiral auxiliary (AA1-D, 2.61 g, 16.0 mmol, 0.9 eq) in THF (80 mL) at −78° C., was added 1.6 M n-BuLi in hexanes (10 mL, 16.0 mmol, 0.9 eq) and this mixture stirred 20 min at −78° C. Then, via cannula, the anhydride solution was added to the mixture containing the chiral auxiliary at −78° C. and the reaction stirred 2 h at room temperature, then saturated NH₄Cl (aq) added. The aqueous phase was extracted with EtOAc (3×). The combined organic phase was washed with brine (1×), dried over MgSO₄, filtered, then the filtrate concentrated in vacuo. The residue was purified by flash column chromatography (gradient, 1:4 to 2:3, Et₂O:hexanes) to provide AA1-E (3.15 g, 68%, white solid).

Step AA1-4. Halogenation. To AA1-E (3.15 g, 12.2 mmol, 1.0 eq) in DCM (94 mL) at −78° C., was added DIPEA (2.55 mL, 19.6 mmol, 1.2 eq) and Bu₂BOTf (3.44 mL, 12.8 mmol, 1.05 eq). The reaction was stirred 10 min at −78° C., then cannulated into a suspension of NBS (2.39 g, 13.4 mmol, 1.1 eq) in DCM (42 mL) at −78° C. The resulting mixture was stirred 2 h at −78° C. and 2 hours at 0° C. To this was added 1M sodium thiosulfate and stirred for 10 min. The aqueous phase was extracted with DCM (3×). The combined organic phase was washed with brine (×1), dried over MgSO₄, filtered, then the filtrate concentrated in vacuo. The residue was immediately (to limit potential decomposition in the crude state) purified by flash column chromatography (100% DCM) to afford AA1-F (667 mg, 17%, white solid).

Step AA1-5. Azide formation. To AA1-F (667 mg, 1.97 mmol, 1.0 eq) in DMSO (20 mL) at room temperature, was added NaN₃ (642 mg, 9.87 mmol, 5.0 eq). The reaction was stirred 1 h at room temperature, then water added. The aqueous phase was extracted with E60 (3×). The combined organic phase was extracted with brine (1×), dried over MgSO₄, filtered, then the filtrate concentrated in vacuo to yield AA1-G (552 mg, 93%) as a white solid.

Step AA1-6. Auxiliary cleavage. To AA1-G (1.45 g, 4.83 mmol, 1.0 eq) in THF/H₂O (3:1, 100 mL) at room temperature, was added LiOH (608 mg, 14.5 mmol, 3.0 eq) and H₂O₂ (30%, 1.38 mL, 24.2 mmol, 5.0 eq). The reaction was stirred at room temperature for 2 h, then the THF evaporated and H₂O added. The aqueous solution was washed with DCM (3×), then acidified to pH=2 with 3N HCl. The acidic aqueous phase was extracted with Et₂O (3×). The combined organic phase was washed with 1M Na₂S₂O₃ (3×), dried over MgSO₄, filtered, then concentrated in vacuo to afford AA1-H (830 mg, 100%) as a colorless oil).

Step AA1-7. Azide reduction. To AA1-H (830 mg, 5.35 mmol, 1.0 eq) in THF/H₂O (2:1, 105 mL) at room temperature, was added 50% wet 10% Pd/C (250 mg, 20% w/w). Hydrogen gas was bubbled directly into this solution for 30 min, then stirred overnight under a hydrogen atmosphere. If reaction was incomplete as indicated by TLC, the catalyst was removed by filtration, a fresh amount of catalyst was added and treated with hydrogen gas in a Parr apparatus for 1 h at 20 psi. When the reaction was completed, it was filtered through a Celite® pad and carefully rinsed with THF/H₂O, then the filtrate evaporated in vacuo to remove THF. (Note that the product sometimes precipitates during the hydrogenation.) The resulting aqueous phase was washed with DCM (3×), then concentrated in vacuo (or alternatively lyophilized) to afford H-(3Me)Cpg-OH (355 mg, 51%) as a grayish solid.

### Example AA2

### Standard Procedure for the Synthesis of H-Anti-(3H,4Me)Cpg-OH

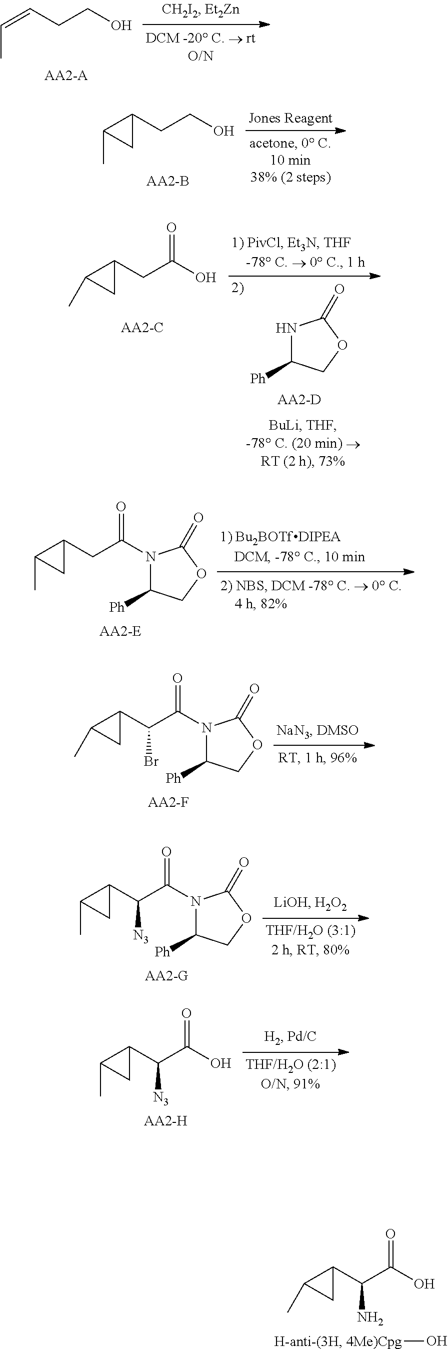

Step AA2-1: Cyclopropanation. To a solution of (Z)-pent-3-en-1-ol (AA2-A, 3.34 g, 38.9 mmol, 1.0 eq) in DCM (390 mL) at −20° C., was carefully added neat diethylzinc (20.0 mL, 194 mmol, 5.0 eq) and diiodomethane (31.4 mL, 398 mmol, 10.0 eq) and temperature quickly raised to 0° C. (CAUTION: Temperature control is very important. Diiodomethane (mp: 5-8° C.) and diethylzinc (mp: −28° C.) can freeze and stop agitation suddenly with a risk of explosion upon melting). The reaction was warmed slowly to room temperature and stirred overnight. Saturated NH₄Cl (aq) was added and the aqueous phase extracted with Et₂O (3×). The combined organic phase was washed with saturated aq. NaHCO₃ (2×), brine(1×), dried over MgSO₄, filtered, then concentrated by rotary evaporator under low temperature and pressure due to the low boiling point of the product to afford 2-(2-methylcyclopropyl)ethanol (AA2-B, 29.5 g, >100%, dark liquid), which was used as obtained in the next step.

Step AA2-2. Oxidation. A solution of AA2-B (38.9 mmol, 1.0 eq) in acetone (390 mL) was cooled to 0° C. Jones reagent was added until the solution remained orange in color, then stirred for an additional 10 min at 0° C. Water was added and the aqueous phase extracted with Et₂O (3×). The combined organic phase was extracted with 1M sodium carbonate 1M (3×). Then, the resulting combined aqueous phase was washed with Et₂O (3×), acidified to pH=2 with 6N HCl at 0° C. and extracted with Et₂O (3×). The combined organic phase was washed with water (1×), brine (1×), dried over MgSO₄, filtered, then the filtrate concentrated in vacuo to provide 2-(1-methylcyclopropyl)acetic acid (AA2-C, 1.7 g, 38% for 2 steps) as a colorless liquid with an unpleasant odor.

Step AA2-3. Chiral auxiliary anchoring. To the chiral auxiliary (AA2-D, 2.19 g, 13.4 mmol, 0.9 eq) in THF (75 mL) at −78° C., was added 1.6 M n-BuLi in hexanes (8.4 mL, 13.4 mmol, 0.9 eq) and the solution stirred 20 min at −78° C. To AA2-C (1.7 g, 14.9 mmol, 1.0 eq) in THF (166 mL) at −78° C., was added Et₃N (2.5 mL, 17.9 mmol, 1.2 eq) and PivCl (2.02 mL, 16.4 mmol, 1.1 eq) in order to form a mixed anhydride and the reaction stirred 15 min at −78° C. and 45 min at 0° C., then cooled down to −78° C. The anhydride solution was added via cannula to the auxiliary mixture at −78° C., then stirred 2 h at room temperature. Saturated NH₄Cl (aq) was added and the aqueous phase extracted with EtOAc (3×). The combined organic phase was washed with brine (1×), dried over MgSO₄, filtered, then the filtrate concentrated in vacuo. The residue was purified by flash column chromatography (gradient, 1:4 to 2:3, Et₂O/hexanes) to yield AA2-E (2.8 g, 73%) as a colorless oil.

Step AA2-4. Halogenation. To AA2-E (2.8 g, 10.8 mmol, 1.0 eq) in DCM (83 mL) at −78° C., was added D1PEA (2.25 mL, 13.0 mmol, 1.2 eq) and Bu₂BOTf (3.05 mL, 11.4 mmol, 1.05 eq), then the mixture stirred 10 min at −78° C. This solution was transferred via cannula to a suspension of NBS (2.11 g, 11.9 mmol, 1.1 eq) in DCM (37 mL) at −78° C., then stirred 2 h at −78° C. and 2 h at 0° C. 1M Sodium thiosulfate was added and the mixture stirred for 10 min. The resulting aqueous phase was washed with DCM (3×). The combined organic phase was washed with brine (1×), dried over MgSO₄, filtered, then the filtrate concentrated in vacuo. The residue was purified immediately to avoid composition in the crude state by flash column chromatography (100% DCM) to afford AA2-F (2.98 g, 82%) as an orange oil.

Step AA2-5. Azide formation. To AA2-F (2.98 g, 8.82 mmol, 1.0 eq) in DMSO (88 mL) at room temperature, was added NaN₃ (2.87 g, 44.1 mmol, 5.0 eq). The mixture was stirred 1 h at room temperature, then water added. The aqueous phase was washed with Et₂O (3×). The combined organic phase was washed with brine (1×), dried over MgSO₄, filtered, then the filtrate concentrated in vacuo to yield AA2-G (2.54 g, 96%) as an orange oil.

Step AA2-6. Chiral auxiliary cleavage. To AA2-G (2.54 g, 8.47 mmol, 1.0 eq) in THF/H₂O (3:1, 180 mL) at room temperature, was added LiOH (1.07 g, 25.4 mmol, 3.0 eq) and 30% H₂O₂ (2.42 mL, 42.4 mmol, 5.0 eq), then the reaction stirred at room temperature for 2 h. The THF was evaporated from the reaction mixture in vacuo, then H₂O added. The aqueous phase was washed with DCM (3×), acidified to pH=2 with 3N HCl. The acidic aqueous phase was washed with Et₂O (3×). The combined organic phase was washed with 1M Na₂S₂O₃ (3×), dried over MgSO₄, filtered, then the filtrate concentrated in vacuo to provide AA2-H (1.05 g, 80%) as a colorless oil.

Step AA2-7. Azide reduction. To AA2-H (1.05 g, 6.77 mmol, 1.0 eq) in THF/H₂O (2:1, 135 mL) at room temperature, was added 50% wet 10% Pd/Cl (300 mg, 20% w/w). Hydrogen gas was bubbled directly into this solution for 30 min and stirred overnight under a hydrogen atmosphere. If reaction is incomplete as indicated by TLC, the catalyst was removed by filtration, a fresh amount of catalyst was added and the reaction treated with hydrogen gas in a Parr apparatus for 1 h at 20 psi. When the reaction was completed, it was filtered through a Celite® pad and carefully rinsed with THF/H₂O, then concentrated in vacuo to remove the THF. (Note that the product sometimes precipitates during the hydrogenation.) The resulting aqueous phase was washed with DCM (3×), then concentrated in vacuo (or alternatively lyophilized) to give H-anti-(3H,4Me)Cpg-OH (794 mg, 91%) as a beige solid.

### Example AA3

### Standard Procedure for the Synthesis of H-syn-(3H,4Me)Cpg-OH

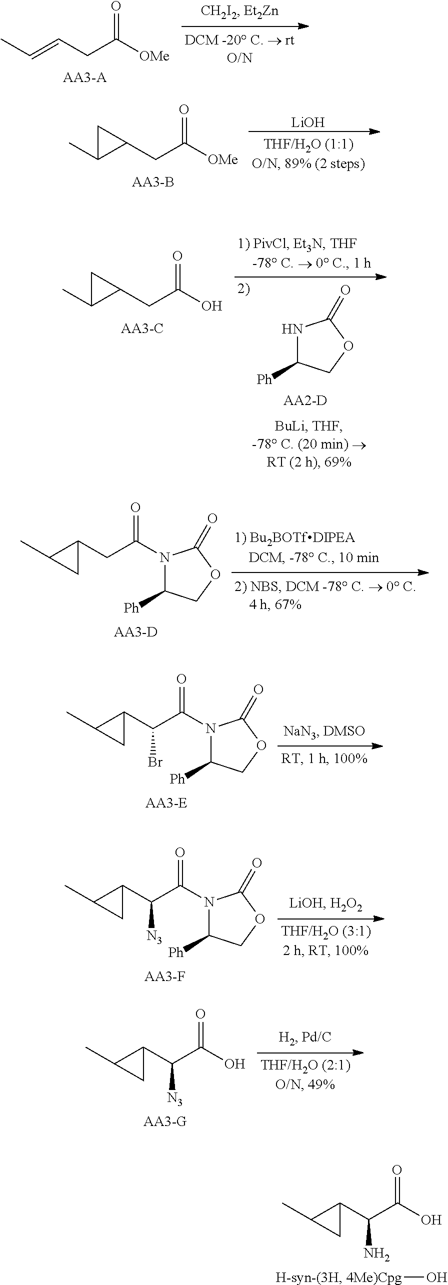

Step AA3-1: Cyclopropanation. To a solution of (E)-pent-3-en-1-ol (AA3-A, 4.77 mL, 38.9 mmol, 1.0 eq) in DCM (390 mL) at −20° C., was carefully added neat diethylzinc (20.0 mL, 194 mmol, 5.0 eq) and diiodomethane (31.4 mL, 398 mmol, 10.0 eq) and temperature quickly raised to 0° C. (CAUTION: Temperature control is very important. Diiodomethane (mp: 5-8° C.) and diethylzinc (mp: −28° C.) can freeze and stop agitation suddenly with a risk of explosion upon melting). The reaction was warmed slowly to room temperature and stirred overnight. Saturated NH₄Cl (aq) was added and the aqueous phase extracted with Et₂O (3×). The combined organic phase was washed with saturated aq. NaHCO₃ (2×), brine(1×), dried over MgSO₄, filtered, then concentrated by rotary evaporator under low temperature (bath T <15° C.) and pressure due to the low boiling point of the product to afford methyl-2-(2-methylcyclopropyl)acetate (AA3-B, 19 g, >100%, dark liquid), which was used as obtained in the next step.

Step AA3-2. Ester hydrolysis. To AA3-B (38.9 mmol, 1.0 eq) in THF/H₂O (1:1, 200 mL) was added LiOH (8.17 g, 194.5 mmol, 5.0 eq) and the reaction stirred overnight. The THF was evaporated in vacuo and the remaining aqueous phase washed with Et₂O (3×). The aqueous phase was acidified to pH 2 with 3 N HCl, then extracted with Et₂O (3×). The combined organic phase was washed with brine (1×), dried with MgSO₄, filter, then the filtrate concentrated under reduced pressure to afford 2-(2-methylcyclopropyl)acetic acid (AA3-C, 3.96 g, 89% for 2 steps) as an orange liquid with an unpleasant odor.

Step AA3-3. Chiral auxiliary anchoring. To the chiral auxiliary (AA2-D, 5.09 g, 31.2 mmol, 0.9 eq) in THF (173 mL) at −78° C., was added 1.6 M n-BuLi in hexanes (19.5 mL, 31.2 mmol, 0.9 eq) and the solution stirred 20 min at −78° C. To AA3-C (3.96 g, 34.7 mmol, 1.0 eq) in THF (386 mL) at −78° C., was added Et₃N (5.8 mL, 41.6 mmol, 1.2 eq) and PivCl (4.71 mL, 38.2 mmol, 1.1 eq) in order to form a mixed anhydride and the reaction stirred 15 min at −78° C. and 45 min at 0° C., then cooled back to −78° C. The anhydride solution was added via cannula to the auxiliary mixture at −78° C., then stirred 2 h at room temperature. Saturated NHCl (aq) was added and the aqueous phase extracted with EtOAc (3×). The combined organic phase was washed with brine (1×), dried over MgSO₄, filtered, then the filtrate concentrated in vacuo. The residue was purified by flash column chromatography (gradient, 1:4 to 2:3, Et₂O/hexanes) to yield AA3-D (6.18 g, 69%) as a white solid.

Step AA3-4. Halogenation. To AA3-D (6.18 g, 23.9 mmol, 1.0 eq) in DCM (184 mL) at −78° C., was added DIPEA (4.99 mL, 28.7 mmol, 1.2 eq) and Bu₂BOTf (6.73 mL, 25.1 mmol, 1.05 eq), then the mixture stirred 10 min at −78° C. This solution was transferred via cannula to a suspension of NBS (4.68 g, 26.3 mmol, 1.1 eq) in DCM (82 mL) at −78° C., then stirred 2 h at −78° C. and 2 h at 0° C. 1M Sodium thiosulfate was added and the mixture stirred for 10 min. The resulting aqueous phase was washed with DCM (3×). The combined organic phase was washed with brine (1×), dried over MgSO₄, filtered, then the filtrate concentrated in vacuo. The residue was purified immediately to avoid composition in the crude state by flash column chromatography (100% DCM) to afford AA3-E (5.41 g, 67%) as a yellow oil.

Step AA3-5. Azide formation. To AA3-E (2.70 g, 7.99 mmol, 1.0 eq) in DMSO (80 mL) at room temperature, was added NaN₃ (2.60 g, 40.0 mmol, 5.0 eq). The mixture was stirred 1 h at room temperature, then water added. The aqueous phase was washed with Et₂O (3×). The combined organic phase was washed with brine (1×), dried over MgSO₄, filtered, then the filtrate concentrated in vacuo to yield AA3-F (2.53 g, 100%,) as a white solid.

Step AA3-6. Chiral auxiliary cleavage. To AA3-F (2.53 g, 8.43 mmol, 1.0 eq) in THF/H₂O (3:1, 168 mL) at room temperature, was added LiOH (1.06 g, 25.3 mmol, 3.0 eq) and 30% H₂O₂ (2.66 mL, 42.1 mmol, 5.0 eq), then the reaction stirred at room temperature for 2 h. The THF was evaporated from the reaction mixture in vacuo, then H₂O added. The aqueous phase was washed with DCM (3×), acidified to pH=2 with 3N HCl. The acidic aqueous phase was washed with Et₂O (3×). The combined organic phase was washed with 1 M Na₂S₂O₃ (3×), dried over MgSO₄, filtered, then the filtrate concentrated in vacuo to provide AA3-G (1.15 g, 80%) as an orange oil.

Step AA3-7. Azide reduction. To AA3-G (1.15 g, 7.42 mmol, 1.0 eq) in THF/H₂O (2:1, 18 mL) at room temperature, was added 50% wet 10% Pd/Cl (230 mg, 20% w/w). Hydrogen gas was bubbled directly into this solution for 30 min and then stirred overnight under a hydrogen atmosphere. If reaction is incomplete as indicated by TLC, the catalyst was removed by filtration, a fresh amount of catalyst was added and the reaction treated with hydrogen gas in a Parr apparatus for 1 h at 20 psi. When the reaction was completed, it was filtered through a Celite® pad and carefully rinsed with THF/H₂O, then concentrated in vacuo to remove the THF. (Note that the product sometimes precipitates during the hydrogenation.) The resulting aqueous phase was washed with DCM (3×), then concentrated in vacuo (or alternatively lyophilized) to give, H-syn-(3H,4Me)Cpg-OH (472 mg, 49%) as a brown solid.

### Example AA4

### Standard Procedure for the Synthesis of H-β-(S)Me-Phe-OH

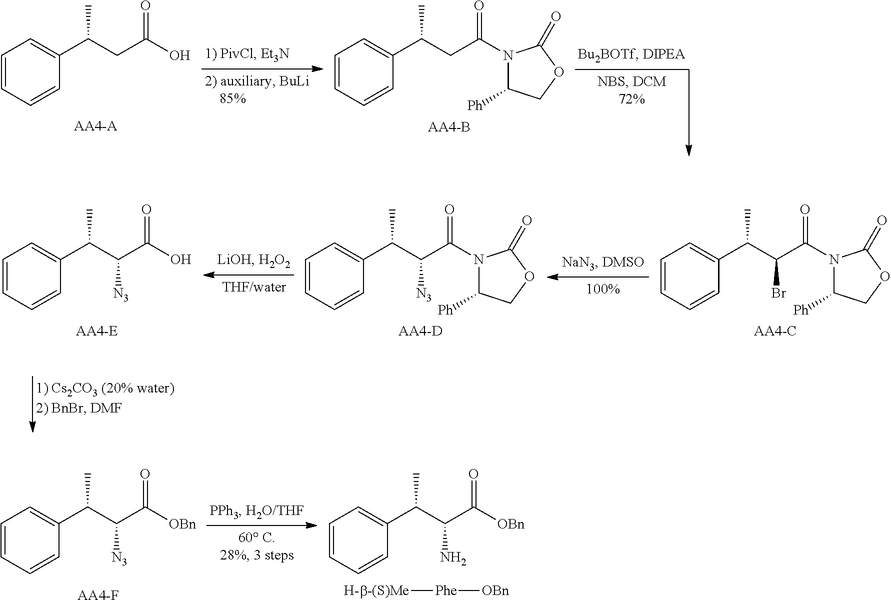

This synthesis was based on the reaction methodology described by Evans for the synthesis of chiral amino acids (Evans, D. A.; Ellman, J. A.; Dorow, R. L. Tetrahedron Lett. 1987, 28, 1123-1126). An asymmetric auxiliary was added to chiral acid AA4-A (1.83 g) using standard methodology to give AA4-B (2.9 g, 85%). Asymmetric bromination to provide AA4-C (2.6 g, 72%, plus 10-15% unreacted AA4-B) was followed by azide S_{N}2 displacement to afford AA4-D (2.3 g, 100%). Cleavage of the auxiliary provided AA4-E, then formation of the benzyl ester gave AA4-F. Reaction with triphenylphosphine to form the iminophosphorane, then hydrolysis with water converted the azide to an amine and gave 500 mg (28%, 3 steps) of the protected amino acid, H-β-(S)Me-Phe-OBn.

### Example AA5

### Standard Procedure for the Synthesis of o-Tyr Lactone (AA5-3)

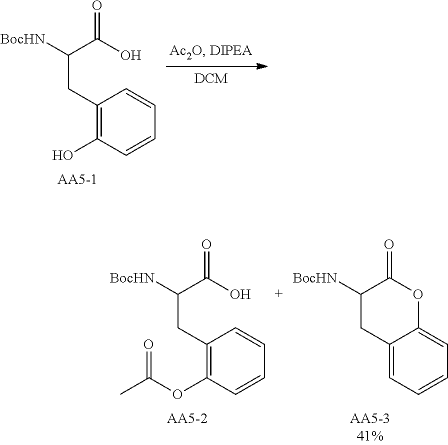

To a solution of Boc-(DL)ₒTyr-OH (AA5-1, 2.76 g, 9.82 mmol, 1.0 eq) in DCM (49 mL) was added DIPEA (3.4 mL, 19.6 mmol, 2.0 eq) followed by Ac₂O (1.02 mL, 10.8 mmol, 1.1 eq). The mixture was stirred for 3 h at RT. Solvent was evaporated in vacuo and the residue dissolved in EtOAc. This organic phase was washed with citrate buffer (1 M, pH 3.5, 3×), brine (1×), dried over MgSO₄, filtered, and the filtrate concentrated under reduced pressure. The residue was purified by flash chromatography [gradient, EtOAc/Hex (1:1) to 100% EtOAc] to give lactone AA5-3 as a white solid (1.06 g, 41%) In addition, 1.06 g of a fraction containing a mixture of AA5-1 and acetylated o-tyrosine (AA5-2) was obtained.

### Example 2

### Synthesis of Tethers

### A. Standard Procedure for the Synthesis of Tether T59

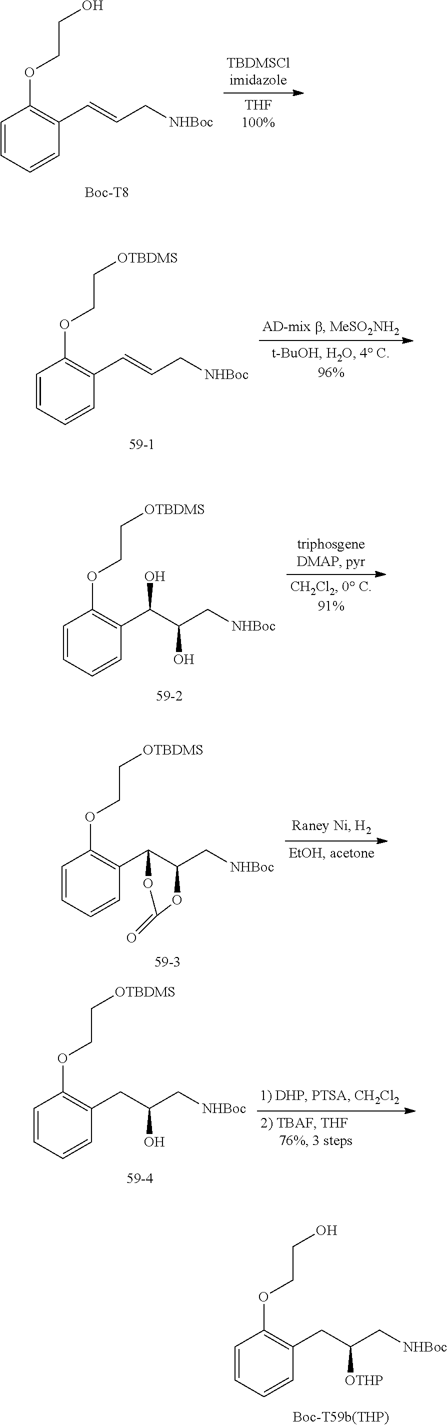

Step T59-1: To a solution of Boc-T8 (32.3 g, 110.2 mmol, 1.0 eq) in THF (500 mL) were added imidazole (15.0 g, 220.4 mmol, 2.0 eq) and TBDMSCl (21.6 g, 143.3 mmol, 1.3 eq) and the mixture stirred 2 h with monitoring by TLC. The solution was then treated with saturated aqueous NH₄Cl and the aqueous phase extracted with EtOAc. The combined organic phase was dried over MgSO₄, filtered and the filtrate concentrated under reduced pressure. The resulting residue was filtered through a silica gel pad (10% EtOAc/90% hexanes) to give 59-1 as a colorless oil (100%).

TLC: R_{f}=0.60 (30% EtOAc/70% hexanes; detection: UV, Mo/Ce).

Step T59-2: To a solution of 59-1 (20.1 g, 49.3 mmol, 1.0 eq) in a mixture of H₂O:t-BuOH (1:1, 500 mL) were added AD-mix β (60 g) and methanesulfonamide (4.7 g, 49.3 mmol, 1.0 eq) and the resulting orange mixture stirred at 4° C. for 36-48 h during which time the color changes to yellow. Once TLC indicated the reaction was complete, sodium sulfite (75 g, 12.0 eq) was added and the mixture stirred at room temperature 1 h. The mixture was extracted with EtOAc, then the combined organic phase extracted with water and brine. The organic phase was dried over MgSO₄, filtered and the filtrate concentrated under reduced pressure. The residue was purified by flash chromatography (50% EtOAc/50% hexanes) to give 59-2 as a yellow oil (96%).

TLC: R_{f}=0.41 (50% EtOAc/50% hexanes; detection: UV, KMnO₄).

Step T59-3: To a solution of 59-2 (20.9 g, 47.4 mmol, 1.0 eq) in DCM (300 mL) at 0° C. were added pyridine (15 mL) and DMAP (293 mg, 2.4 mmol, 0.05 eq). Triphosgene (14.1 g, 47.4 mmol, 1.0 eq) in DCM (50 mL) was then slowly added to this mixture. The reaction was stirred at 0° C. for 45 min at which time TLC indicated the reaction was completed. The solution was treated with saturated aqueous NH₄Cl and the organic phase separated. The aqueous phase was extracted with Et₂O and the combined organic phase extracted with saturated aqueous NH₄Cl. The organic phase was dried over MgSO₄, filtered and the filtrate concentrated under reduced pressure. The resulting residue was filtered through a silica gel pad (30% EtOAc/70% hexanes) to give 59-3 as a yellow oil (91%).

TLC: R_{f}=0.56 (50% EtOAc/50% hexanes; detection: UV, Mo/Ce).

Step T59-4: To a solution of 59-3 (20.2 g, 43.3 mmol, 1.0 eq) in a mixture of 95% EtOH:acetone (3:1, 400 mL) was added Raney Ni (50% in water, 51 mL, 433 mmol, 10.0 eq). Hydrogen was bubbled into this solution for 6 h with monitoring by TLC. When the reaction was completed, N₂ was bubbled through the mixture to remove excess hydrogen, then the mixture filtered though a Celite pad and rinsed with EtOAc. Concentration of the filtrate under reduced pressure gave 59-4 as a colorless oil sufficiently pure to be used for the next step.

TLC: R_{f}=0.29 (30% EtOAc/70% hexanes; detection: UV, Mo/Ce).

Step T59-5: To a solution of the alcohol 59-4 (17.0 g, 40.0 mmol, 1.0 eq) in CH₂Cl₂ (250 mL) were added DHP (4.4 mL, 48.0 mmol, 1.2 eq) and PTSA (380 mg, 2.0 mmol, 0.05 eq). The mixture was stirred at room temperature for 1 h. Upon completion as indicated by TLC (30% EtOAc/70% hexanes; detection: UV, Mo/Ce; R_{f}=0.51), the solution was treated with saturated aqueous NaHCO₃, then the aqueous phase extracted with CH₂Cl₂. The combined organic phase was dried over MgSO₄, filtered and the filtrate concentrated under reduced pressure. The residue was dissolved in THF (250 mL) and a 1M solution of TBAF in THF (80.0 mL, 80.0 mmol, 2.0 eq) added. The mixture was stirred at rt for 1 h. When TLC indicated the reaction was complete, the mixture was treated with brine the layers separated, and the aqueous phase extracted with EtOAc. The combined organic phase was dried over MgSO₄, filtered and the filtrate concentrated to dryness under reduced pressure. The residue was purified by flash chromatography (50% EtOAc/50% hexanes) to give Boc-T59b(THP) as a yellow oil (76%, 3 steps).

TLC: R_{f}=0.12 (30% EtOAc/70% hexanes; detection: UV, Mo/Ce);

¹³C NMR (CDCl₃, ppm): δ 19.5, 25.5, 25.6, 28.6, 30.8, 31.1, 33.5, 44.5, 61.5, 62.6, 69.9, 75.0, 96.7, 111.0, 120.9, 121.0, 128.1, 131.8, 156.9.

To obtain Boc-T59a and its THP-protected derivative, the same procedure as above was followed, but utilizing AD-mix α, with the yields for the sequence being comparable. Other suitable protecting groups in place of THP can be introduced in the last step as well.

### B. Standard Procedure for the Synthesis of Tether T104b

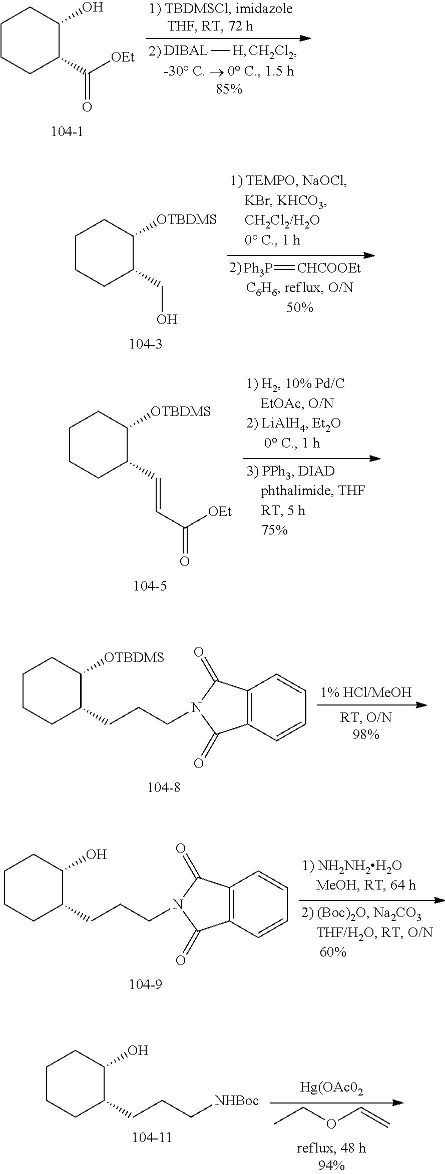

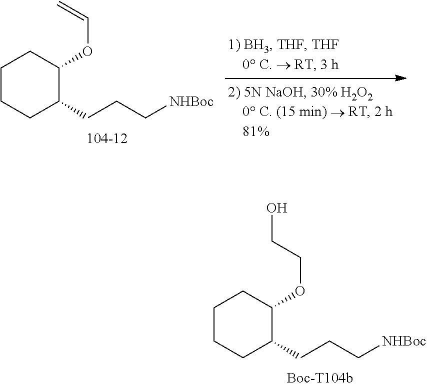

Step T104-1. To a solution of ethyl (1R,2S)-cis-2-hydroxy-cyclohexanoate 104-1 (obtained from Julich, now Codexis, product no. 15.60, 50 g, 290 mmol) in THF (500 mL) was added imidazole (29.6 g, 435 mmol) and TBDMSCl (49.8 g, 331 mmol). The reaction was stirred at RT for 72 h and then quenched with saturated NH₄Cl (aq). The mixture was extracted with Et₂O (3×). The organic phases were combined, dried over MgSO₄, filtered, and the filtrate concentrated under reduced pressure to yield the intermediate protected ester (104-2, 93 g), which was used directly in the next step.

Step T104-2. 104-2 (215 g, 0.75 mol) obtained from the previous step was dissolved in DCM (2 L) and the solution cooled to −30° C. To this solution was added DIBAL-H (1 M solution in DCM, 2250 mL, 2.25 mol) over a period of 1.5 h. The reaction mixture was stirred 1 h at 0° C. and then poured into an aqueous solution of Rochelle salts (2 M, 4 L) at 0° C. This mixture was vigorously stirred overnight at RT, then extracted with DCM (3×). The combined organic phase was washed with brine, dried over MgSO₄, filtered, and the filtrate concentrated under reduced pressure to give 155 g of 104-3 (85%).

Step T104-3. To a solution of 104-3 (196 g, 0.8 mol) in CH₂Cl₂ (2 L) at 0° C. was added TEMPO (12.5 g, 80 mmol) followed by an aqueous solution of KHCO₃ (1.6 M, 862 g) and an aqueous solution of KBr (2.7 M, 196 g). The mixture was vigorously stirred and an 11% NaOCl aqueous solution (573 mL, 1.04 mol, 1.3 eq) added over 45 min. When the addition was completed, the mixture was stirred for an additional 15 min at 0° C., then quenched with an aqueous solution of 1 M Na₂S₂O₃ (1 L). The mixture was extracted and the aqueous phase washed with CH₂Cl₂ (2×500 mL). The combined organic phase was dried over MgSO₄, filtered, and the filtrate concentrated under reduced pressure to afford the intermediate aldehyde (104-4, 190 g), which was used in the next step without further purification.

Step T104-4. 104-4 (116 g, 480 mmol) and ethyl triphenylphosphoranylidene carbonate (250 g, 720 mmol) were dissolved in benzene (2 L) and the reaction heated to reflux overnight. The mixture was cooled to RT and evaporated to 50% volume. Hexanes was added, the mixture stirred for 15 min with precipitation of the Ph₃P═O byproduct, then filtered through a pad of silica gel and rinsed with 10% EtOAc/hexanes. The filtrate was concentrated to dryness under reduced pressure to provide 104-5 (125 g, 50%).

Step T104-5. To 104-5 (200 g, 640 mmol) dissolved in EtOAc (3 L) was added 10% Pd/C (50% wet, 68 g) and H₂ bubbled into the mixture for 16 h. The mixture was filtered through a pad of Celite and the filter cake rinsed with EtOAc (1 L). The combined filtrate and washings were concentrated under reduced pressure, then the residue (104-6, 180 g) dissolved in Et₂O. The solution was cooled to 0° C., LiAlH₄ (16.3 g, 430 mmol) added portion-wise, and the mixture stirred for 1 h at 0° C. The reaction was quenched by slowly adding water (17 mL), followed by 15% NaOH aqueous solution (17 mL), and finally water (51 mL). This mixture was stirred 1 h at 0° C., then filtered. The filtrate was concentrated in vacuo to give the intermediate alcohol (104-7, 152.6 g). This alcohol was dissolved in THF (3 L) and triphenylphosphine (220.6 g, 841 mmol), phthalimide (123.7 g, 841 mmol) and DIAD (154.5 mL, 785 mmol) added. The mixture was stirred 5 h at RT, then the solvent evaporated under reduced pressure. The residue was dissolved in MTBE, stirred for 1 h at RT during which the Ph₃P═O byproduct precipitated, then filtered. The filtrate was evaporated under reduced pressure and the residue purified by flash chromatography (gradient, 5% Et₂O/hexanes to 20% Et₂O/hexanes) to give 104-8 (194 g, 75%).

Step T104-6. 104-8 (194 g, 483 mmol) was dissolved in a solution of 1% HCl/MeOH (3 L). This solution was stirred at RT overnight, then quenched with water (1.5 L). The mixture was extracted with DCM (2×1.5 L) and the combined organic fractions dried over MgSO₄, filtered, and the filtrate concentrated under reduced pressure. The residue was passed through a pad of silica gel and rinsed with 10% Et₂O/hexanes to remove the silanol byproduct, then with Et₂O until no additional compound was eluting as evidenced by TLC. The solvents were removed under reduced pressure to yield 104-9 (138.5 g, 98%) as a white solid.

Step T104-7. To a solution of 104-9 (135 g, 470 mmol) in MeOH (3 L) was added hydrazine (88 mL, 1.41 mol). This mixture was stirred at RT for 64 h, then filtered and the filter cake rinsed with EtOH (500 mL). The filtrate and washings were combined and evaporated under reduced pressure. The residue was dissolved in EtOH (1 L), filtered again, and the filter rinsed with EtOH (250 mL). The filtrate and washings were combined and evaporated to dryness under reduced pressure. The residue was redissolved with EtOH (1 L) and again evaporated to dryness in vacuo. The residue was then dissolved in DCM, filtered and the filter rinsed with DCM. The combined filtrate and washings were evaporated to dryness under reduced pressure to give the intermediate amino alcohol 104-10, which was dissolved in a 1:1 mixture of THF/water (3 L). To this mixture were added Na₂CO₃ (150 g, 1.41 mol) followed by (Boc)₂O (153.8 g, 705 mmol). The reaction was stirred overnight at RT and quenched with water. The mixture was extracted with Et₂O (3×). The combined organic phase was washed with brine, dried over MgSO₄, filtered, and the filtrate concentrated to dryness under reduced pressure. The resulting residue was purified by flash chromatography (gradient, 15% Et₂O/Hexanes to 50% Et₂O/Hexanes) to provide 104-11 (73 g, 60%) as an oil.

Step T104-8. To a solution of 104-11 (13.8 g, 53.7 mmol) in ethyl vinyl ether (500 mL) was added mercuric acetate (5.13 g, 16.1 mmol) and the solution heated at reflux for 24 h. Another 0.3 eq of mercuric acetate was then added and the solution again heated at reflux for another 24 h. The solution was cooled to RT, quenched with an aqueous saturated solution of Na₂CO₃ and extracted with Et₂O (3×). The combined organic phases were washed with brine, dried over MgSO₄, filtered, and the filtrate concentrated to dryness under reduced pressure.

The residue was purified by flash chromatography (10% Et₂O/hexane containing 2% Et₃N) to yield 104-12 as a colorless oil (8.6 g, 94%).

Step T104-9. To a solution of 104-12 (13.2 g, 46.6 mmol) in THF (400 mL) at 0° C. was slowly, over a period of 15 min, added a 1 M solution of BH₃.THF (69.9 mL, 69.9 mmol). The mixture was stirred 1 h at 0° C., then 2 h at RT. The solution was cooled to 0° C. and a 5 N solution of NaOH (90 mL) added, followed by a 30% aqueous solution of H₂O₂ (200 mL). The mixture was stirred 15 min at 0° C., then 2 h at RT. The solution was extracted with Et₂O (3×). The combined organic phase was washed with brine, dried over MgSO₄, filtered, and the filtrate concentrated to dryness under reduced pressure. The resulting residue was purified by flash chromatography (30% EtOAc/hexanes) to afford Boc-T104b (11.4 g, 81%).

HPLC/MS: Gradient A4, t_{R}=7.05 min, [M+H]⁺ 302.

The enantiomeric tether Boc-T104a can be accessed similarly using ethyl (1S,2R)-cis-2-hydroxy-cyclohexanoate 104-13.

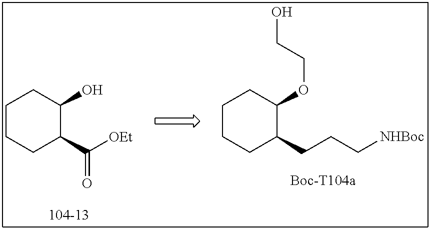

### C. Alternative Procedure for the Synthesis of Tether T104b

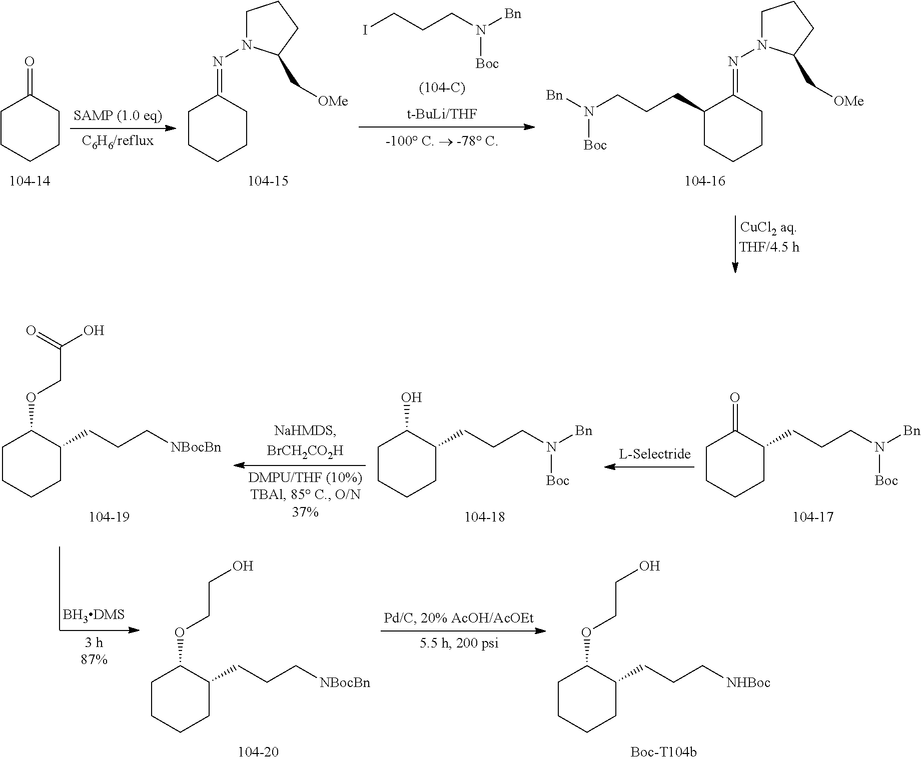

An alternative synthetic route to T104b involves as a key step the asymmetric alkylation of cyclohexanone derivatized with (S)-1-amino-2-methoxymethylpyrrolidine (SAMP) hydrazone as the chiral auxiliary (Enders, D. Alkylation of Chiral Hydrazones. In Asymmetric Synthesis; Morrison, J. D., Ed.; Academic Press: Orlando, Fla., 1984; Vol. 3, pp 275-339.) and 104-C as the electrophile. 104-16 thus obtained was subjected sequentially to hydrazone cleavage and L-Selectride reduction to give the alcohol 104-18. O-Alkylation with bromoacetic acid, borane reduction, then hydrogenolysis of the benzyl protecting group gave Boc-T104b.

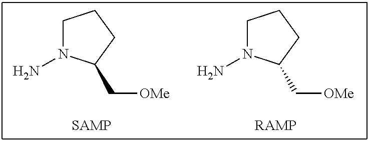

A similar sequence, but using (R)-1-amino-2-methoxymethylpyrrolidine (RAMP) hydrazone as the chiral auxiliary, was utilized to provide Boc-T104a in comparable yields.

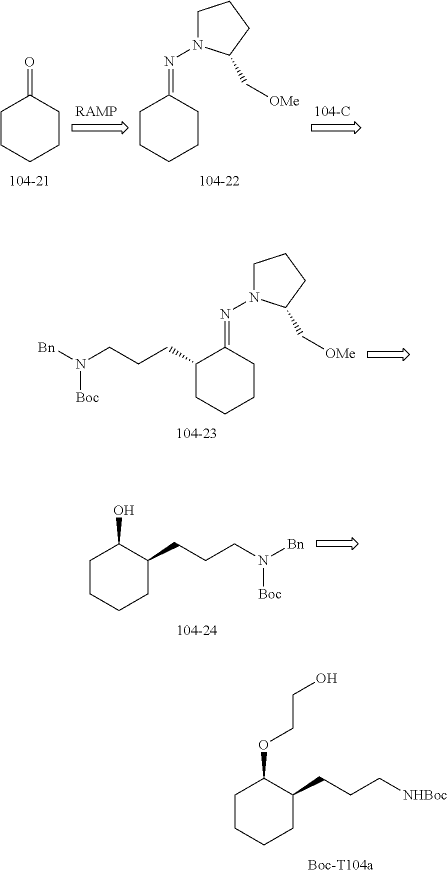

### D. Standard Procedure for the Synthesis of Tether T134

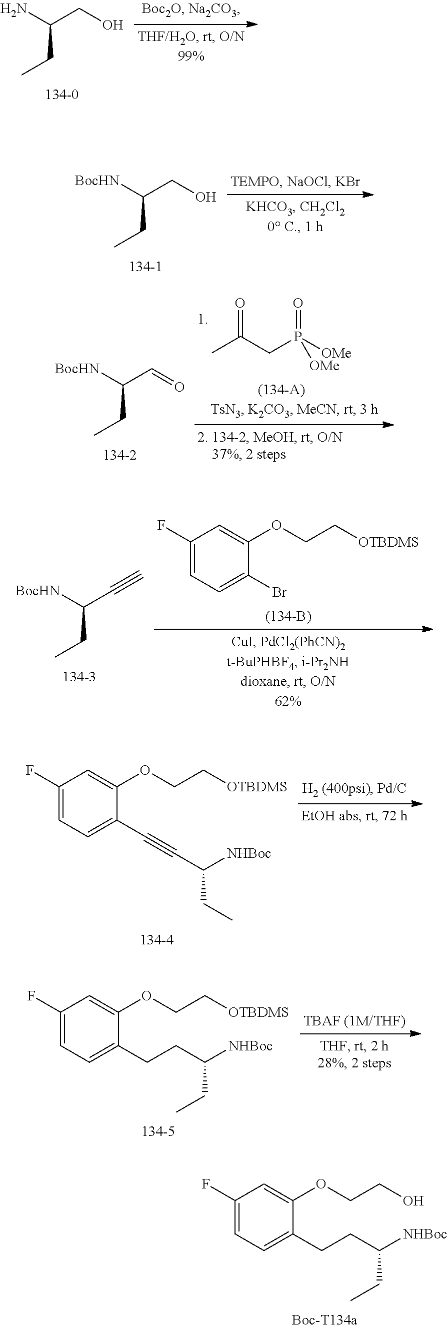

Step T134-1. To a solution of (R)-(−)-2-amino-1-butanol (134-0, 50 g, 561 mmol, 1.0 eq) in THF/water (1:1, 2.8 L) were added (Boc)₂O (129 g, 589 mmol, 1.05 eq) and Na₂CO₃ (71.3 g, 673 mmol, 1.2 eq) and the solution stirred overnight. THF was removed in vacuo and the aqueous phase was extracted with ether (3×500 mL). The combined organic phase was washed with 1M citrate buffer (200 mL) and brine (200 mL), dried with MgSO₄, filtered and concentrated under vacuum. The crude was purified on silica gel (dry pack, 50% EtOAc/Hexanes) to give 134-1 (104.9 g, 554 mmol, 99%) as a colorless oil.

Step T134-2: To a solution of 134-1 (93.8 g, 496 mmol, 1.0 eq) in CH₂Cl₂ (1.24 L) at 0° C. was added TEMPO (7.75 g, 49.6 mmol, 0.1 eq), followed by a 2.75M aqueous solution of KBr (130 g) and a 1.6M solution of KHCO₃ (570 g). NaOCl (11.5%/water, 420 mL, 645 mmol, 1.3 eq) was then added dropwise over ˜30 min with vigorous stirring. The reaction was stirred 10 min at 0° C., then a 1M solution of Na₂S₂O₃ (aq, 400 mL) added to quench excess of oxidant. The mixture was stirred 5 min at 0° C. and warmed to rt over 90 min. The phases were separated and the aqueous phase extracted with CH₂Cl₂ (2×1 L). The combined organic phase was washed with water (1 L) and brine (500 mL), dried with MgSO4, filtered, then the filtrate concentrated under vacuum to give 134-2 (95 g, 508 mmol, >100%) as an orange oil, which was used without further purification for the next step.

Step T134-3: To a solution of tosyl azide (117.3 g, 595 mmol, 1.2 eq, Org. Synth. Coll. Vol. 5, p. 179 (1973); Vol. 48, p 36 (1968)) in MeCN (7.4 L) at 0° C. was added K₂CO₃ (206 g, 1.4 9 mol, 3 eq), followed by 134-A (98.8 g, 595 mmol, 1.2 eq). The reaction was warmed to rt and stirred for 3 h. The crude 134-2 from the previous step in MeOH (1.5 L) was then added and the reaction stirred overnight. The solvents were evaporated in vacuo and water (1.5 L) and Et₂O (1 L) added to the residue. The phases were separated and the aqueous phase extracted with Et₂O (2×1 L). The combined organic phase was washed with water (200 mL) and brine (200 mL), dried with MgSO₄, filtered, then the filtrate concentrated under vacuum. The residue was triturated with pentane (5×500 mL), then the solvent from the triturations concentrated under vacuum. The resulting residue was purified by flash chromatography (gradient, 5-10% EtOAc/hexanes) to give 134-3 (33.7 g, 184 mmol, 37% for 2 steps).

Step T134-4: Into a solution of 134-3 (20.2 g, 110 mmol, 1.7 eq) and bromo-alcohol 134-B (22.6 g, 64.8 mmol, 1.0 eq) in MeCN (325 mL) was bubbled argon for 20 min. Recrystallized CuI (248 mg, 1.30 mmol, 0.02 eq), PdCl₂(PhCN)₂ (744 mg, 1.94 mmol, 0.03 eq), t-Bu₃PHBF₄ (1.22 g, 4.21 mmol, 0.065 eq) and iPr₂NH (16 mL, 110 mmol, 1.7 eq) were then added. The reaction was stirred under an argon atmosphere for 40 h at rt. The reaction was filtered through a silica gel pad and the pad rinsed with EtOAc. The volatiles were removed in vacuo and the residue purified by flash chromatography (gradient, 5-10-20% EtOAc/hexanes) to afford 134-4 (18.3 g, 40.5 mmol, 62%) as a mixture of starting bromide, alkyne and other unknown impurities.

Step T134-5: To alkyne 134-4 (18.2 g, 40.5 mmol, 1.0 eq) in absolute EtOH (300 mL) was added 10% Pd/C (50% wet, 4.29 g, 0.02 eq). The mixture was placed in a Parr reactor under a pressure of 400 psi of hydrogen for 72 h. The reaction can be monitored by HPLC. The mixture was filtered through a Celite® pad then concentrated under vacuum. The residue was dissolved in THF and 1M TBAF in THF (48 mL, 48 mmol) added. The reaction was stirred 2 h at rt then solvent evaporated in vacuo. The resulting residue was purified by flash chromatography (gradient, 10-15-20-30-40-50% acetone/hexanes) to give a mixture of the fully (134-5) and partially reduced products (7.8 g, 22.9 mmol, 57%). This mixture was then dissolved in absolute EtOH (115 mL) and 10% Pd/C (50% wet, 2 g, 0.04 eq) added. The reaction was stirred overnight under H₂ (400 psi) in a Parr reactor. The solution was filtered through a Celite pad and the filtrate evaporated under vacuum. The residue was purified by flash chromatography (gradient, 10-20% acetone/hexanes) to give T134 (5.51 g, 15.1 mmol). Note that 2-(3-fluorophenoxy)ethanol was often present as an impurity in this product. To remove this material, the impure product was dissolved in HCl/MeOH (10% w/w) and agitated 24 h, then the volatiles removed in vacuo. The residue was dissolved in water (100 mL), then washed with MTBE (4×25 mL) until TLC confirmed removal of the 2-(3-fluorophenoxy)ethanol impurity. THF (100 mL) was added followed by Na₂CO₃ to adjust the pH to 10. Excess Boc₂O was added and the solution stirred overnight. The THF was evaporated under vacuum and the aqueous phase extracted with MTBE (3×100 mL). The combined organic phase was dried with MgSO₄, filtered, then the filtrate concentrated under vacuum to obtain a residue that was purified by flash chromatography (gradient, 20-40% acetone/hexanes) to give clean Boc-T134a (3.87 g, 11.3 mmol, 28%, 2 steps) as an oil.

HPLC/MS: Gradient A4, t_{R}=7.39 min, M⁺ 341;

¹H NMR (DMSO, 300 MHz): δ 7.13-7.06 (m, 1H), 6.82 (dd, 1H, J=2.5, 11.5 Hz), 6.68-6.56 (m, 2H), 4.83 (t, 1H, J=5.5 Hz), 3.98 (t, 2H, J=5.1 Hz), 3.72 (dd, 2H, J=5.5, 10.3 Hz), 3.32-3.20 (m, 1H), 2.60-2.40 (m, 2H), 1.66-1.22 (m, 4H), 1.39 (s, 9H), 0.79 (t, 3H, J=7.4 Hz).

The enantiomeric tether T135b is constructed starting from the enantiomer of 134-0.

### E. Standard Procedure for the Synthesis of Tether T135

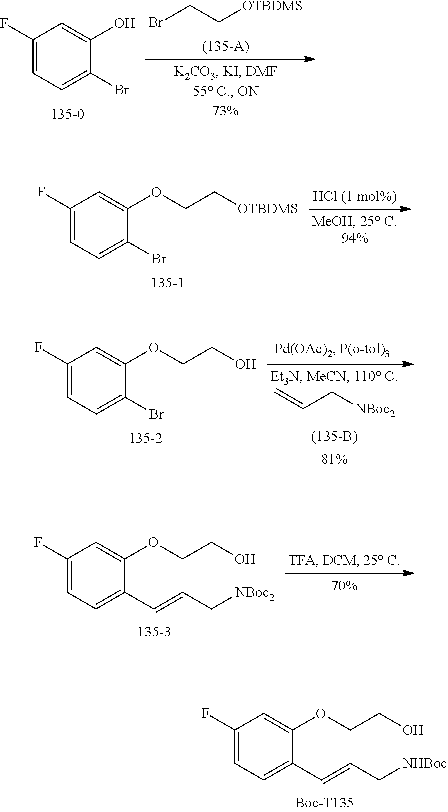

Step T135-1. To a solution of 2-bromo5-fluorophenol (135-0, 15.0 g, 78.5 mmol, 1.0 eq) and 135-A (30.2 g, 126.4 mmol, 1.6 eq) in DMF (Drisolv, 225 mL) are added potassium carbonate (13.0 g, 93.5 mmol, 1.2 eq), potassium iodide (2.5 g, 15.1 mmol, 0.19 eq). The solution was heated to 55° C. and stirred overnight under nitrogen. The solvent was concentrated to dryness under reduced pressure, then the residual oil was diluted with water (200 mL) and extracted with Et₂O (3×150mL). The organic phases are combined and washed with 1 M citrate buffer (2×), brine (1×), dried with magnesium sulfate, filtered, and the filtrate evaporated under vacuum. The crude product was purified by flash chromatography (10% EtOAc/pentane) to give 135-1 as a yellowish solid. (20.0 g, 73%)

TLC: R_{f}=0.68 (25% EtOAc/Hex; detection: UV, CMA).

Step T135-2. To a solution of 135-1 (17.0 g, 48.7 mmol, 1.0 eq) in MeOH (Drisolv, 162 mL) was added HCl (12.1 M, 25 μL, 0.486 mmol, 1 mol %) and the reaction stirred 2.5 h at rt. H₂O was then added and the aqueous layer washed with Et₂O (2×300 mL). The organic layers were combined, washed with saturated aqueous NH₄Cl (300 mL), brine (300 mL), dried over MgSO₄, filtered, and the filtrate concentrated under reduced pressure to leave an orange oil. Purification by flash chromatography (40% EtOAc/Hex) afforded 10.7 g (94%) of 135-2 as a colorless oil.

TLC: R_{f}=0.57 (30% EtOAc/Hex; detection: UV, KMnO₄);

¹H NMR (300 MHz, CDCl₃): δ 7.48 (dd, J=6.3, 8.7 Hz, 1H), 6.58-6.68 (m, 2H), 4.12 (m, 2H), 4.01 (m, 2H), 2.17 (br, 1H).

Step T135-3. In a flame dried flask, MeCN (26 mL) was introduced and degassed with multiple argon/vacuum cycles for 30 min. Then, Pd(OAc)₂ (143 mg, 0.640 mmol, 0.05 eq), P(o-tol)₃ (388 mg, 1.27 mmol, 0.10 eq), diBoc-allylamine (135-B, see procedure following, 3.6 g, 14.0 mmol, 1.1 eq), Et₃N (3.6 mL, 25.5 mmol, 2 eq) and alcohol 135-2 (3.0 g, 12.8 mmol, 1.0 eq) were added. The solution was stirred at rt, quickly degassed, then heated to reflux at 110° C. for 20 h under an argon atmosphere. The reaction mixture was allowed to cool to rt, quenched with H₂O (20 mL), and the layers separated. The aqueous layer was washed with Et₂O (2×60 mL). The organic layers were combined, washed with saturated aqueous NH₄Cl (70 mL), brine (70 mL), dried over MgSO₄, filtered, and the filtrate concentrated under vacuum to give the crude product. Purification by flash chromatography (gradient, 30% to 40% Et₂O/Hex) afforded 4.25 g (81%) of 135-3 as a pale yellow solid.

TLC: R_{f}=0.39 (30% Et₂O/Hex; detection: UV, KMnO₄);

HPLC/MS: Gradient A4, t_{R}=8.55 min, [M+Na]⁺ 434;

¹H NMR (300 MHz, CDCl₃): δ 7.35 (dd, J=6.9, 8.7 Hz, 1H), 6.79 (d, J=15.9 Hz, 1H), 6.56-6.68 (m, 2H), 6.17 (dt, J=undetermined, 15.9 Hz, 1H), 4.31 (dd, J=1.2, 6.3 Hz, 2H), 4.05-4.09 (m, 2H), 3.94-3.98 (m, 2H), 2.26 (br m, 1H), 1.51 (s, 18H).

Step T135-4. To a solution of 135-3 (4.25 g, 10.3 mmol, 1.0 eq) in DCM (Drisolv, 52 mL) under nitrogen, TFA (1.15 mL, 15.5 mmol, 2.0 eq) was added and the solution stirred at rt for 1.75 h with TLC monitoring. Additional TFA (0.5 or 1 eq) was added if reaction was incomplete. The solvent was evaporated under reduced pressure, and the resulting oil purified by flash chromatography with preadsorption on silica (gradient, 40% to 50% Et₂O/hexanes) to yield 2.2 g (70%) of Boc-T135 as a white solid. TLC: R_{f}=0.46 (40% Et₂O/Hex; detection: UV, KMnO₄); HPLC/MS: Gradient A4, t_{R}=6.63 min, [M+Na]⁺ 334;

¹H NMR (300 MHz, CDCl₃): δ 7.15-7.087 (m, 1H), 6.74-6.52 (m, 4H), 4.74 (s (br), 1H), 4.13-4.09 (m, 2H), 4.00-3.97 (m, 2H), 3.92 (t (br), J=5 Hz, 2H), 1.93 (s (br), 1H), 1.46 (s, 9H).

### F. Standard Procedure for the Synthesis of Reagent 135-B

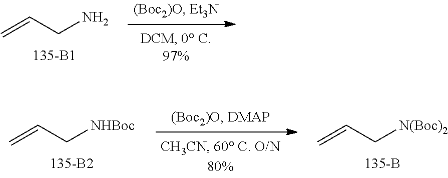

Step T135-5. (Boc)₂O (112 g, 0.531 mol) was added by portions over 2 h to a solution of allylamine (30 g, 0.526 mol) and triethylamine (95 mL, 0.684 mol) in DCM (900 mL) at 0° C., then the solution stirred O/N. The reaction mixture was washed successively with citrate buffer (pH 3.5, 3×), NaHCO₃ (2×) and brine (2×), dried over anhydrous MgSO₄, filtered, and the filtrate evaporated under vacuum to give 80.5 g (97%) of 135-B1.

TLC: R_{f}: 0.35 (30/70 EtOAc/FIex; detection: UV, KMnO₄).

Step T135-6. To a solution of 135-B1 (80.5 g, 0.513 mol) in CH₃CN (1.8 μL) were added (Boc)₂O (134.2 g, 0.615 mol) and DMAP (4.39 g, 0.036 mol). The mixture was heated 0/N at 60° C. The solvent was removed and the crude compound was purified by dry pack (10% EtOAc/Hex) to provide 135-B as a white solid (105 g, 80%).

TLC: R_{f}: 0.27 (30/70 EtOAc/Hex; detection: UV, KmnO₄);

¹H NMR (300 MHz, CDCl₃): δ 5.78-5.90 (1H, m); 5.09-5.20 (2H, m); 4.17 (2H, dt, J=5.5 and 1.5 Hz); 1.5 (9H,$).

### G. Standard Procedure for the Synthesis of Tether T136

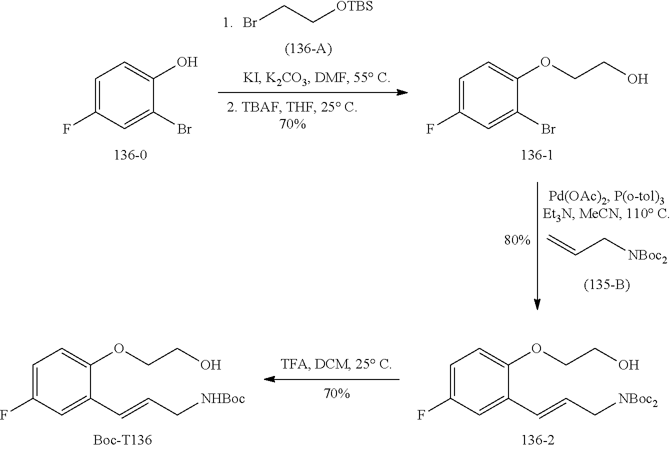

Step 136-1. To a solution of 2-bromo-4-fluorophenol (136-0, 30.0 g, 158 mmol, 1.0 eq) and protected bromoethanol (136-A, 41.4 g, 173.8 mmol, 1.1 eq) in DMF (Drisolv, 320 mL) were added potassium carbonate (28.0 g, 205.4 mmol, 1.3 eq), potassium iodide (5.24 g, 31.6 mmol, 0.2 eq) at rt. The solution was heated to 55° C. and stirred overnight under nitrogen. The mixture was allowed to cool to rt and H₂O (400 mL) added. The resulting solution was washed with Et₂O (3×300 mL). The combined organic layer was washed successively with H₂O (2×300 mL), saturated aq. NH₄Cl (300 mL), brine (300 mL), dried over MgSO₄, filtered, and the filtrate evaporated to dryness under vacuum. The crude product thus obtained was used without further purification for the next step, but could be purified by flash chromatography (10% Et₂O/Hex) to give the alkylated phenol as a colorless solid (79 mmol scale, 27.3 g, 99%).

TLC: R_{f}=0.69 (10% Et₂O/Hex; detection: UV, CMA).

Step 136-2. To a solution of crude product from Step 136-1 (55.1 g, 158 mmol, 1.0 eq) in THF (320 mL) was added TBAF (1 M solution in THF, 237 mL, 237 mmol, 1.5 eq). The reaction was stirred overnight at rt, then H₂O (300 mL) added and the layers separated. The aqueous phase was washed with EtOAc (2×300 mL). The combined organic layer was washed with saturated aq. NH₄Cl (300 mL), brine (300 mL), dried over MgSO₄, filtered, and the filtrate concentrated to dryness under reduced pressure. The crude product was purified by flash chromatography (40% EtOAc/Hex) to afford 26.0 g (70%, 2 steps) of 136-1 as a pale orange solid (in other batches, 136-1 was obtained as a colorless solid).

TLC: R_{f}=0.34 (40% EtOAc/Hex; detection: UV, KMnO₄).

Step 136-3. To a flame-dried flask, MeCN (130 mL) was introduced and degassed with multiple argon-vacuum cycles for 30 min. Then, Pd(OAc)₂ (715 mg, 3.19 mmol, 0.05 eq), P(o-tol)₃ (1.94 g, 6.38 mmol, 0.10 eq), diBoc-allylamine (135-B, 18.0 g, 70.2 mmol, 1.1 eq), Et₃N (18 mL, 127 mmol, 2 eq) and 136-1 (15.0 g, 63.8 mmol, 1.0 eq) were added. The solution was stirred at it and quickly degassed, then heated at 110° C. for 20 h under argon. The reaction mixture was allowed to cool to rt, quenched with H₂O (100 mL), the layers separated, and the aqueous layer washed with Et₂O (2×90 mL). The combined organic layers was washed with saturated aq. NH₄Cl (100 mL), brine (100 mL), dried over MgSO₄, filtered, and the filtrate concentrated to dryness under vacuum to give the crude product which was used with no further purification for the next step, but could be purified by flash chromatography (gradient, 30% to 40% Et₂O/Hex) to yield 11.6 g (80%, 35 mmol scale) of 136-2 as a pale yellow solid.

TLC: R_{f}=0.37 (30% EtOAc/Hex; detection: UV, KMnO₄).

Step 136-4. To a solution of crude 136-2 (26.2 g, 63.8 mmol, 1.0 eq) in DCM (Drisolv, 320 mL) under nitrogen, TFA (9.5 mL, 127.6 mL, 2.0 eq) was added. The solution was stirred at rt for 1.75 h with TLC monitoring. Upon completion, the solvent was evaporated under reduced pressure, and the resulting oil purified by flash chromatography with preadsorption on silica (40% Et₂O/Hex) to afford 10.2 g (51% for 2 steps) of Boc-T136. In a separate experiment, 6.1 g (70%, 28.2 mmol scale) of Boc-T136 was obtained as a pale yellow solid.

TLC: R_{f}=0.29 (40% Et₂O/Hex; detection: UV, KMnO₄);

HPLC/MS: Gradient A4, t_{R}=6.62 min, [M+Na]⁺ 334;

¹H NMR (300 MHz, CDCl₃): δ 7.08 (dd, J=3, 9 Hz, 1H), 6.89-6.76 (m, 3H), 6.17 (dt, J=6, 16 Hz, 1H), 4.81 (s (br), 1H), 4.06-4.02 (m, 2H), 3.96-3.93 (m, 2H), 3.88 (m (br), 2H), 2.71 (s (br), 1H), 1.45 (s, 9H).

### H. Standard Procedure for the Synthesis of Tether T137

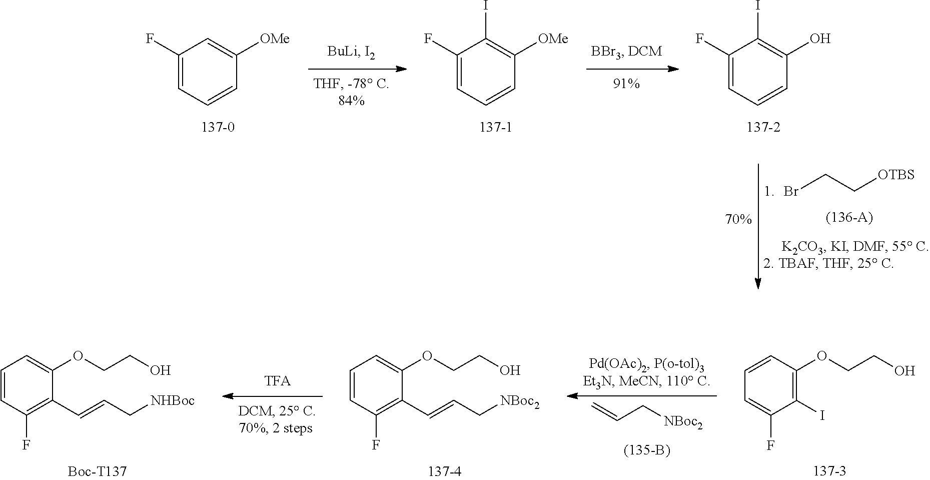

Step T137-1. To a solution of n-BuLi (1.6 M in hexane, 82.0 mL, 130.8 mmol, 1.1 eq) in THF (dry, freshly distilled from Na-benzophenone ketyl, 450 mL) was added a solution of 3-fluoroanisole (137-0, 15.0 g, 118.9 mmol, 1.0 eq) in THF (dry, 45 mL) diopwise at −78° C. under N₂ (over ˜25 min). The solution was stirred at −78° C. for 30 min. A solution of I₂ (36.1 g, 142.7 mmol. 1.2 eq) in THF (dry, 100 mL) was then added dropwise at −78° C. (addition time: 30 min, the addition funnel was rinsed with THF at the end of the addition). The solution was allowed to warm to −60° C. and stirred 45 min with TLC monitoring of the reaction progress. When reaction was complete, H₂O (100 mL) was added carefully at −60° C., followed by Na₂SO₃ (10% w/v; 100 mL), and the mixture stirred for 5 min. The aqueous phase was washed with hexane (3×). The combined organic phase was washed with NaHSO₃ (10% w/v; 2×), H₂O (2×), dried over anhydrous MgSO₄, filtered, and the filtrate concentrated under reduced pressure to afford a yellow residue. Purification by flash chromatography (10% EtOAc/Hex) gave 25.3 g (84%) of 137-1 as a colorless oil. The crude product could also be used directly for the next step of the sequence.

TLC: R_{f}=0.34 (5% EtOAc/Hex; detection: UV, Mo/Ce);

HPLC/MS: Gradient A4, t_{R}=6.64 min, M⁺ 252.

Step T137-2. To a solution of 137-1 (25.0 g, 99.2 mmol, 1.0 eq) in DCM (Drisolv, 100 mL) was added a solution of BBr₃ in DCM (1.0 M, 248 mL, 248 mmol, 2.5 eq) dropwise at −30° C. under N₂ (over ˜30 min). The solution was stirred at −30° C. for 3 h, then allowed to warm to rt overnight. The mixture was cooled to 0° C. and MeOH carefully added dropwise (gas generation), followed by addition of H₂O. The cooling bath was removed and the mixture stirred for 10 min at room temperature. The aqueous layer was separated and washed with DCM. The organic layers were combined, washed with brine (300 mL), dried over anhydrous MgSO₄, filtered, and the filtrate concentrated under reduced pressure to give a black residue. Purification by flash chromatography (20% EtOAc/Hex) affords 21.5 g (91%) of 137-2 as a brown oil. The crude oil could also be used directly for the next step of the sequence.

TLC: R_{f}=0.35 (20% EtOAc/Hex; detection: UV, KMnO₄);

HPLC: Gradient B4, t_{R}=7.02 min.

Step T137-3. To a solution of 137-2 (18.8 g, 79.07 mmol, 1.0 eq) and protected bromoethanol (136-A, 20.8 g, 87.0 mmol, 1.1 eq) in DMF (Drisolv, 320 mL) were added potassium carbonate (14.2 g, 102.8 mmol, 1.3 eq), potassium iodide (2.62 g, 15.8 mmol, 0.2 eq) at it The solution was heated to 55° C. and stirred overnight under N₂. The mixture was allowed to cool to rt and H₂O (500 mL) added. The layers were separated and the aqueous layer washed with Et₂O (3×300 mL). The organic layers were combined, washed with H₂O (2×300 mL), saturated aq. NH₄Cl (300 mL), brine (300 mL), dried over MgSO₄, filtered, and the filtrate concentrated under reduced pressure. The crude oil thus obtained was used with no further purification for the next step.

Step T137-4. To a solution of the crude oil from step T137-3 (31.0 g, 79.07 mmol, 1.0 eq) in MeOH (263 mL) was added HCl (12.1 M, 65 μL, 0.79 mmol, 0.01 eq). The reaction was stirred 2.5 h at rt, then H₂O added and the layers separated. The aqueous layer was washed with Et₂O (2×300 mL). The organic layers were combined, washed with saturated aq. NH₄Cl (300 mL), brine (300 mL), dried over MgSO₄, filtered, and the filtrate concentrated under reduced pressure to give an orange oil. Purification by flash chromatography (40% EtOAc/Hex) afforded 26.0 g (70%, 2′ steps) of 137-3 as a white solid.

TLC: R_{f}=0.38 (50% MTBE/Hex; detection: UV, CAM).

Step T137-5. Into a flame dried flask, MeCN (92 mL) was introduced and degassed with multiple argon-vacuum cycles for 30 min. Then, Pd(OAc)₂ (516 mg, 2.30 mmol, 0.05 eq), P(o-tol)₃ (1.40 g, 4.61 mmol, 0.10 eq), diBoc-allylamine (135-B, 13.0 g, 50.7 mmol, 1.1 eq), Et₃N (13.0 mL, 92.18 mmol, 2 eq) and alcohol 137-3 (13.0 g, 46.1 mmol, 1.0 eq) were added. The solution was stirred at rt and quickly degassed, then heated to 110° C. for 20 h under argon. The reaction mixture was allowed to cool to rt, quenched with H₂O (150 mL) and the layers separated. The aqueous layer was washed with Et₂O (2×90 mL). The organic layers were combined, washed with saturated aq. NH₄Cl (100 mL), brine (100 mL), dried over MgSO₄, filtered, and the filtrate concentrated under vacuum to give crude 137-4 which was used without further purification for the next step, but could be purified by flash chromatography (gradient, 30% to 40% Et₂O/Hex).

TLC: R_{f}=0.35 (30% Et₂O/Hex; detection: UV, KMnO₄);

HPLC: Gradient A4, t_{R}=8.54 min.

Step T137-6. To a solution of crude 137-4 (7.0 g, 17.0 mmol, 1.0 eq) in DCM (Drisolv, 90 mL) under nitrogen, TFA (1.90 mL, 127.6 mL, 2.0 eq) was added and the solution stirred at rt for 1.75 h with TLC monitoring. More TFA (0.5 eq) could be added if reaction was not complete. When complete, the solvent was evaporated under reduced pressure, and the resulting oil purified by flash chromatography with pre-adsorption on silica (gradient, 40% to 50% Et₂O/Hex) to afford 3.71 g (70%) of Boc-T137 as a white solid after trituration with hexanes.

TLC: R_{f}=0.30 (40% Et₂O/Hex; detection: UV, KMnO₄);

HPLC/MS: Gradient A4, t_{R}=6.71 min, [M+Na]⁺ 334;

¹H NMR (300 MHz, CDCl₃): δ 7.15-7.087 (m, 1H), 6.74-6.52 (m, 4H), 4.74 (s (br), 1H), 4.13-4.09 (m, 2H), 4.00-3.97 (m, 2H), 3.92 (t (br), J=5 Hz, 2H), 1.93 (s (br), 1H), 1.46 (s, 9H).

### I. Standard Procedure for the Synthesis of Tether T138

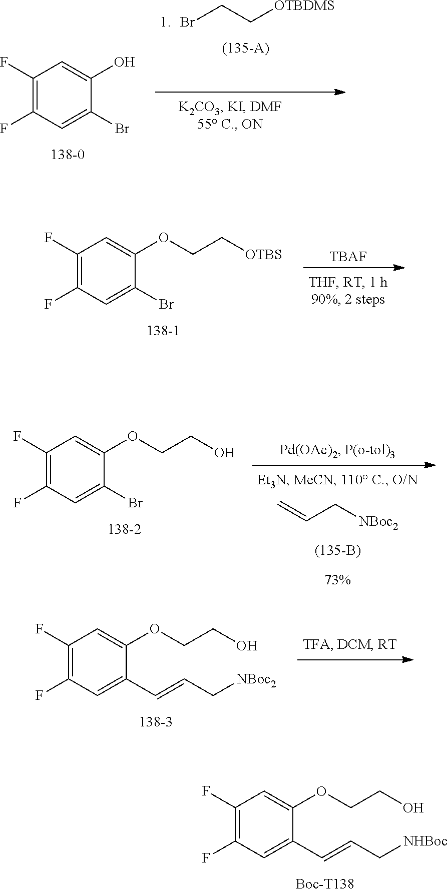

Step T138-1. To a solution of 2,3-difluoro-6-bromophenol (138-0, 25 g, 120 mmol, 1.0 eq) and 135-A (48.6 g, 239 mmol, 1.7 eq) in dry DMF (535 mL) were added potassium carbonate (19.8 g, 144 mmol, 1.2 eq) and potassium iodide (4.0 g, 24 mmol, 0.2 eq). The solution was heated to 55° C. and stirred overnight under nitrogen. The solvent was removed under reduced pressure until dryness, then the residual oil diluted with water and extracted with diethyl ether (3×). The organic phase were combined and washed with citrate buffer (2×) and with brine (1×). The organic phase was dried over anhydrous MgSO₄, filtered, and the filtrate concentrated under vacuum to give 138-1 as a brown solid (32 g), which was used without further purification for the next step.

TLC: R_{f}: 0.83 (30%/70% EtOAc/Hex); detection: UV, KMnO₄);

HPLC/MS: Gradient A4, t_{R}=13.87 min, [M+H+2]⁺ 369.

Step T138-2. To a solution of 138-1 (30.2 g, 120 mmol, 1.0 eq) in THF (600 mL), TBAF (1.0 M solution in THF, 240 mL, 240 mmol, 2.0 eq) was added. The reaction was stirred for 1 h at RT. The mixture was diluted with diethyl ether, washed with saturated aqueous ammonium chloride solution (1×) and brine (1×). The organic phase was dried over anhydrous MgSO₄, filtered, and the filtrate concentrated under vacuum. The residue was purified by flash chromatography (25% EtOAc/Hex) to provide 138-2 as a colorless oil (27.2 g, 90%, 2 steps).

TLC: R_{f}: 0.27 (30%/70% EtOAc/Hex); detection: UV, KMnO₄);

HPLC: Gradient A4, t_{R}=5.73 min.

Step T138-3. A solution of 138-2 (10.63 g, 40.0 mmol, 1.0 eq) in acetonitrile (84 mL) was degassed using the following cycle: vacuum, nitrogen, vacuum, nitrogen. To this were added palladium acetate (472 mg, 0.05 eq) and P(o-tol)₃ (1.38 g, 0.1 eq). The mixture was degassed once again, then triethylamine (11.8 mL, 79 mmol, 2.0 eq) and 135-B (11.8 g, 43 mmol, 1.1 eq) added. The solution was stirred at 110° C., O/N. Water was then added and the aqueous phase extracted with ethyl acetate (4×). The combined organic phase was washed with water and brine, dried over MgSO₄, filtered, and the filtrate concentrated under reduced pressure. The residue thus obtained was purified by flash chromatography (30% EtOAc/Hex) to yield 138-3 as a golden syrup (12.4 g, 73%).

TLC: R_{f}: 0.28 (40%/60% EtOAc/Hex); detection: UV, KMnO₄);

HPLC/MS: Gradient A4, t_{R}=9.06 min, [M+Na]⁺ 452.

Step T138-4. To a solution of 138-3 (11.53 g, 27.0 mmol, 1.0 eq) in DCM (135 mL) under nitrogen was added TFA (3.0 mL, 40 mmol, 1.5 eq). The reaction was stirred at RT until completion and then the solvent evaporated to dryness under reduced pressure. The residue was purified by flash chromatography (30% EtOAc/Hex) to give Boc-T138 as a yellow solid.

TLC: R_{f}: 0.25 (40%/60% EtOAc/Hex); detection: UV, KMnO₄);

HPLC/MS: Gradient A4, t_{R}=6.83 min, [M]⁺ 329, [2M+H]⁺ 559;

¹H NMR (CDCl₃): δ 7.2 (1H, dd, J=11.2 and 8.9 Hz); 6.77 to 6.66 (2H, m); 6.13 (1H, dt, J=15.9, 6.2 Hz); 4.71 (1H, bs); 4.06 to 4.01 (2H, m); 4.01 to 3.93 (2H, m); 3.92 to 3.85 (2H, m); 2.21 (1 h, bs); 1.46 (9H, s).

### J. Standard Procedure for the Synthesis of Tether T139

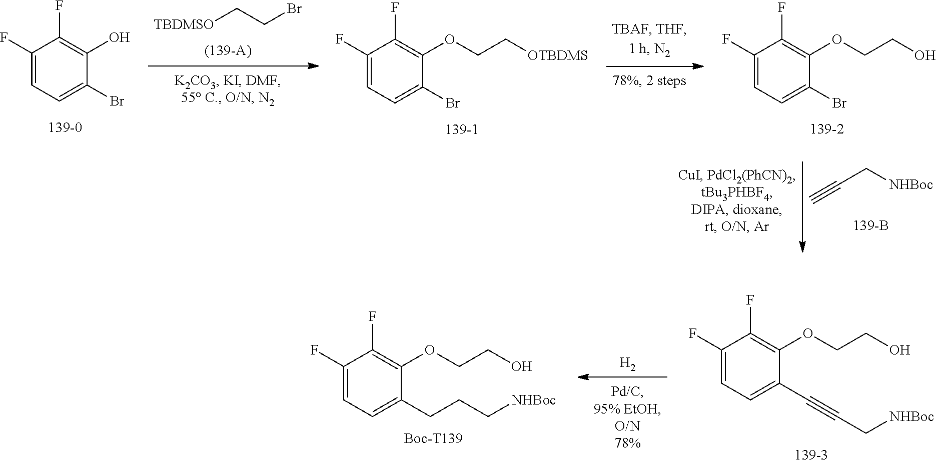

Step T139-1: To a solution of bromide 139-0 (25 g, 120 mmol, 1.0 eq) and protected bromoethanol 139-A (48.6 g, 239 mmol, 1.7 eq) in dry DMF (535 mL) were added potassium carbonate (19.8 g, 144 mmol, 1.2 eq) and potassium iodide (4.0 g, 24 mmol, 0.2 eq). The solution was heated to 55° C., then stirred overnight under nitrogen. The solvent was removed under reduced pressure, then the residual oil diluted with water and extracted with Et₂O (3×). The organic phases were combined and washed with citrate buffer (2×) and brine (1×). The organic phase was dried over anhydrous MgSO₄, filtered, then the filtrate concentrated under vacuum. The crude product 139-1 (32 g) was thus obtained as a brown solid and used without further purification for the next step.

TLC: R_{f}: 0.83 (30/70 EtOAc/Hex; detection: UV, KMnO₄);

HPLC/MS: Gradient A4, t_{R}=13.87 min, [M+2]⁺ 368.

Step T139-2: To a solution of 139-1 (30.2 g, 120 mmol, 1.0 eq) THF (600 mL), TBAF (1.0 M solution in THF, 240 mL, 240 mmol, 2.0 eq) was added. The reaction was stirred for 1 h at room temperature. The mixture was then diluted with Et₂O, washed with saturated aqueous ammonium chloride solution (2×) and brine (1×). The organic phase was dried over anhydrous MgSO₄, filtered, then the filtrate concentrated under vacuum. The crude residue was purified by flash chromatography (25% EtOAc/Hex) to give the alcohol 139-2 as a colorless oil (27.2 g, 90% 2 steps).

TLC: R_{f}: 0.27 (30/70 EtOAc/Hex; detection: UV, KMnO₄).

Step T139-3:. Into a solution of alcohol 139-2 (10 g, 40 mmol, 1.0 eq), Boc-propargylamine 139-B (10.4 g, 68 mmol, 1.7 eq) in dioxane (ACS grade, 40 mL) was bubbled argon for 15-20 min. Then, tBu₃PHBF₄ (454 mg, 0.03 eq), recrystallized copper (I) iodide (150 mg, 0.02 eq), dichlorobis(benzonitrile) palladium (II) (150 mg, 0.02 eq) and diisopropylamine (9.5 mL, 67 mmol, 1.7 eq) were added and the reaction mixture stirred at rt overnight under argon. The solution was diluted with EtOAc, filtered through a silica gel pad and washed with ethyl acetate until no more material was eluting. The filtrate was concentrated under reduced pressure, then the crude residue purified by flash chromatography (30% EtOAc/Hex to give the alkyne 139-3 as a golden syrup (8.3 g, 70%).

TLC: R_{f}: 0.28 (30/70 EtOAc/Hex; detection: UV, KMnO₄);

HPLC/MS: Gradient A4, t_{R}=6.71 min, M⁺ 327.

Step T139-4: To a solution of alkyne 139-3 (8.3 g, 25 mmol, 1.0 eq) in 95% ethanol (241 mL) under nitrogen was added palladium on carbon (5.7 g, 50% water) and then hydrogen bubbled into the mixture overnight. When the reaction was complete as indicated by ¹H NMR, nitrogen was bubbled through the mixture for 10 min to remove excess hydrogen. The solvent was filtered through a Celite pad and washed with ethyl acetate until no further material was eluting. The filtrate was concentrated under reduced pressure. The resulting crude residue was purified by flash chromatography (30% EtOAc/Hex) to give Boc-T139 as a yellowish oil (7.65 g, 90%).

TLC: R_{f}: 0.13 (25/75 EtOAc/Hex; detection: UV, ninhydrin);

HPLC/MS: Gradient A4, t_{R}=6.91 min, M⁺ 331;

¹H NMR (300 MHz, CDCl₃): δ 6.85-7.0 (m, 1H,), 6.6-6.7 (m, 1H,), 4.9-5.0 (m, 1H), 3.95-4.1 (m, 4H), 3.15-3.2 (m, 2H), 2.9-3.0 (m, 1H), 2.55-2.65 (m, 2H), 1.75-1.95 (m, 2H), 1.45 (s, 9H).

### K. Standard Procedure for the Synthesis of Tether T140

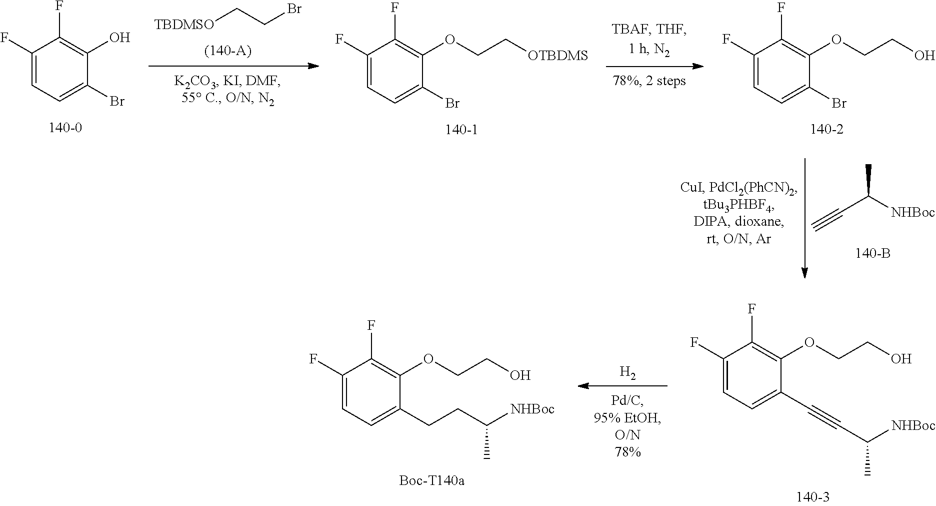

Step T140-1. To a solution of bromide 140-0 (25 g, 120 mmol, 1.0 eq) and protected bromoethanol 140-A (48.6 g, 239 mmol, 1.7 eq) in dry DMF (535 mL) were added potassium carbonate (19.8 g, 144 mmol, 1.2 eq) and potassium iodide (4.0 g, 24 mmol, 0.2 eq). The solution was heated to 55° C. and stirred overnight under nitrogen. The solvent was removed under reduced pressure until dryness, then the residual oil diluted with water and extracted with Et₂O (3×). The organic phases were combined, washed with 1M citrate buffer (2×) and brine (1×), dried over anhydrous MgSO₄, filtered, then the filtrate concentrated under vacuum. The crude product 140-1 (32 g) thus obtained was a brown solid and used without further purification for the next step.

TLC: R_{f}: 0.83 (30/70 EtOAc/Hex; detection: UV, KMnO₄);

HPLC/MS: Gradient A4, t_{R}=13.87 min, [M+2]⁺ 368.

Step T140-2. To a solution of crude protected alcohol 140-1 (30.2 g, 120 mmol, 1.0 eq) in THF (600 mL) was added TBAF (1.0 M solution in THF, 240 mL, 240 mmol, 2.0 eq). The reaction was stirred for 1 h at rt. The reaction mixture was diluted with Et₂O, washed with saturated ammonium chloride solution (2×) and brine (1×). The organic phase was dried over anhydrous MgSO₄, filtered, then the filtrate concentrated under vacuum. The crude residue was purified by flash chromatography (25% EtOAc/Hex) to give the alcohol 140-2 as a colorless oil (27.2 g, 90% for 2 steps).

TLC:R_{f}: 0.27 (30/70 EtOAc/Hex; detection: UV, KMnO₄).

Step T140-3. To a solution of alcohol 140-2 (9.5 g, 38 mmol, 1.0 eq) and 140-B (10.82 g, 64 mmol, 1.7 eq) in dioxane (ACS grade, 38 mL) was bubbled argon for 15-20 min. Then, tBu₃PHBF₄ (707 mg, 0.07 eq), recrystallized copper (I) iodide (143 mg, 0.02 eq), dichlorobis(benzonitrile) palladium (II) (431 mg, 0.03 eq) and diisopropylamine (9.5 mL, 67 mmol, 1.7 eq) were added and the reaction mixture was stirred at rt overnight under argon. The solution was diluted with EtOAc, filtered through a silica gel pad and washed with ethyl acetate until no more material was eluting. The solvent was removed under reduced pressure, then the crude product purified by flash chromatography (30% EtOAc/Hex) to give the alkyne 140-3 as a golden syrup. (6.5 g, 54%).

TLC: R_{f}: 0.28 (30/70 EtOAc/Hex; detection: UV, KMnO₄);

HPLC/MS: Gradient A4, t_{R}=7.01 min, M⁺ 341.

Step T140-4. To a solution of alkyne 140-3 (6.2 g, 18 mmol, 1.0 eq) in 95% ethanol (171 mL) under nitrogen was added palladium on carbon (4.04 g, 50% water), then hydrogen gas bubbled into it overnight. When the reaction was complete as indicated by ¹H NMR, nitrogen was bubbled through the reaction for 10 min to remove the excess hydrogen. The solvent was filtered through a Celite pad and washed with ethyl acetate until no more material was eluting. The filtrate was concentrated under reduced pressure and the crude product purified by flash chromatography (30% EtOAc/Hex) to give Boc-T140a as a yellowish oil (4.63 g, 75%).

TLC: R_{f}: 0.13 (25/75 EtOAc/Hex; detection UV, ninhydrin);

HPLC/MS: Gradient A4, t_{R}=7.81 min, M⁺ 345;

¹H NMR (300 MHz, DMSO): δ 6.8-7.0 (m, 1H,), 6.0-6.7 (m, 1H,), 4.5-4.65 (m, 1H), 3.85-4.1 (m, 4H), 3.55-3.75 (m, 1H), 3.2-3.35 (m, 1H), 2.6-2.7 (m, 1H), 2.4-2.6 (m, 1H), 1.8-2.0 (m, 1H) 1.45 (s, 9H), 1.15 (d, 3H, J=6.6 Hz).

Use of 140-C, the enantiomer of 140-B, in the same sequence can be used to provide the enantiomeric tether Boc-T140b.

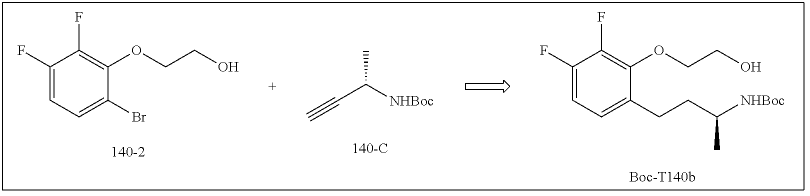

### L. Standard Procedure for the Synthesis of Tether T141

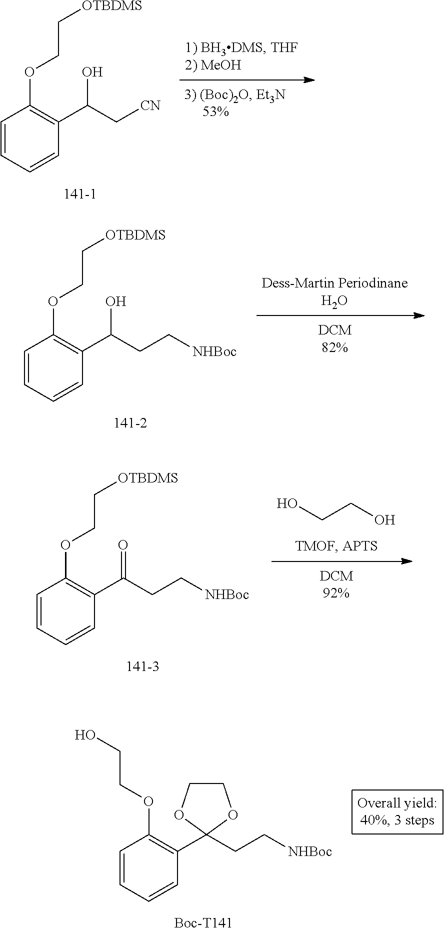

Step T141-1. To a solution of the nitrile 141-1 (6.0 g, 18.7 mmol, 1.0 eq) in THF (915 mL) was added a solution of 10 M BH₃.DMS (2.8 mL, 28.1 mmol, 1.5 eq) and the resulting mixture stirred at reflux overnight. Progress of the reaction was monitored by TLC (20% EtOAc/Hex; detection: UV, ninhydrin; the product amine was at the baseline). Once completed, the solution was cooled to 0° C. and MeOH added slowly to quench the excess BH₃. The mixture was stirred 1 h at rt, then Et₃N (3.9 mL, 28.1 mmol, 1.5 eq) and (Boc)₂O (5.1 g, 22.4 mmol, 1.2 eq) added. The resulting mixture was stirred at rt 3 d with monitoring of the reaction by TLC (20% EtOAc/Hex; detection: UV, ninhydrin; R_{f}=0.15). A saturated aqueous solution of NH₄Cl was then added slowly and the layers separated. The aqueous phase was extracted with EtOAc and the combined organic phase was dried over MgSO₄, filtered and the filtrate concentrated in vacuo. The residue was purified by flash chromatography (gradient, 20% to 40% EtOAc/Hex) to give 141-2 as yellow oil (4.8 g, 53%).

HPLC/MS: Gradient A4, t_{R}=11.86 min, [M+H]⁺ 426.

Step T141-2. To a solution of 141-2 (1.7 g, 4.00 mmol, 1.0 eq) in DCM (20 mL) were added H₂O (81 μL, 4.50 mmol, 1.125 eq) and Dess-Martin periodinane (2.1 g, 5.0 mmol, 1.25 eq). The resulting mixture was stirred at rt 25 min. Progress of the reaction was monitored by TLC (15% EtOAc/Hex; detection: UV, Mo/Ce; R_{f}=0.48.) An aqueous sodium thiosulfate solution (10%, 25 mL) was added slowly. The aqueous phase was separated and the organic phase washed with aqueous sodium thiosulfate (10%, 2×25 mL)., dried over MgSO₄, filtered, and the filtrate concentrated under reduced pressure. The residue was purified by flash chromatography (gradient, 5% to 15% EtOAc/Hex) to provide 141-3 as colorless oil (1.4 g, 82%).

HPLC/MS: Gradient A4, t_{R}=12.38 min, [M+Na]⁺ 446.

Step T141-3. To a solution of 141-3 (1.4 g, 3.30 mmol, 1.0 eq) in DCM (26 mL) were added trimethyl orthoformate (1.1 mL, 9.90 mmol, 3 eq), ethylene glycol (1.8 mL, 33.0 mmol, 10 eq) and APTS (62 mg. 0.33 mmol, 0.1 eq). The resulting mixture was stirred at rt for 20 h. Progress of the reaction was monitored by TLC (40% EtOAc/Hex; detection: UV, Mo/Ce; R_{f}=0.14.) and HPLC. A saturated aqueous solution of NaHCO₃ (30 mL) was added and the resulting aqueous phase extracted with DCM (3×30 mL). The combined organic phase was dried over MgSO₄, filtered, and the filtrate concentrated in vacuo. The residue was purified by flash chromatography (gradient, 40% to 60% EtOAc/Hex) to give the Boc-T141 as a colorless oil (1.1 g, 92%).

HPLC/MS: Gradient A4, t_{R}=6.45 min, M⁺ 353;

¹H MR (CDCl₃, ppm): δ 7.42 (dd, J=7.61, 1.76 Hz, 1H), 7.27 (dt, J=7.79, 7.76, 1.80 Hz, 1H), 7.00-6.85 (m, 2H), 4.97 (br, 1H), 4.20-3.65 (m, 9H), 3.17 (dd, J=12.04, 5.98 Hz, 2H), 2.34 (t, J=6.43, 6.43 Hz, 2H), 1.42 (s, 9H).

### M. Standard Procedure for the Synthesis of Tether T142

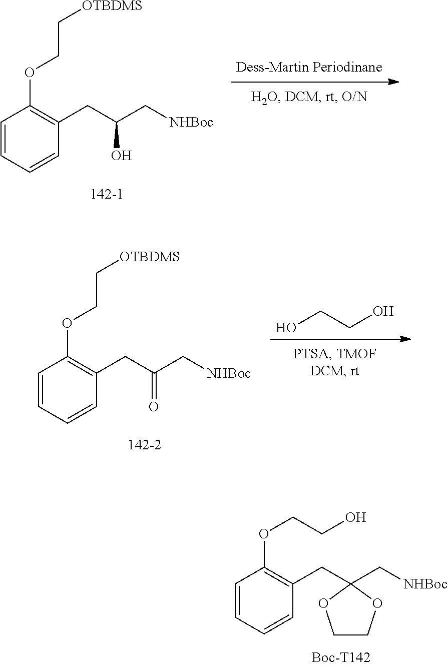

Step 142-1. To a solution of 142-1 (4.2 g, 9.9 mmol, 1.0 eq) in DCM (49.5 mL) was added H₂O (200 μL, 11.1 mmol 1.13 eq) and Dess-Martin periodinane (6.28 g, 14.8 mmol, 1.5 eq). The reaction was stirred 2 h at it. A second portion of Dess-Martin periodinane was added (1.05 g, 2.5 mmol, 0.25 eq) was added and the reaction was stirred an additional 2 h. The resulting white precipitate was removed by filtration and rinsed with DCM. The filtrate and rinses were combined and washed with an aqueous solution of 10% sodium thiosulfate, dried over MgSO₄, filtered, and the filtrate concentrated to dryness in vacuo. The residue was purified by flash chromatography (gradient, 10% to 15% to 20% EtOAc/Hex) to obtain 142-2 as a white solid (3.4 g, 82.8%).

HPLC/MS: Gradient A4, t_{R}=12.17 min, [M+Na]⁺ 446.

Step 142-2. To a solution of 142-2 (3.46 g, 8.2 mmol, 1.0 eq), trimethylorthoformate (2.7 mL, 24.5 mmol, 3.0 eq) and ethylene glycol (4.8 mL, 81.8 mmol, 10.0 eq) in DCM (41 mL) was added PTSA (154 mg, 0.81 mmol, 0.1 eq) and the reaction stirred for 4 h at rt. An aqueous solution of NaHCO₃ (satd.) was added and the organic phase separated. The aqueous phase was extracted with DCM (2×) and the combined organic phase dried over MgSO₄, filtered, and the filtrate removed in vacuo. The residue was purified by flash chromatography (gradient, 40%, 50%, 60% 75% EtOAc/Hex) to provide Boc-T142 as a white solid (2.18 g, 75.6%).

HPLC/MS: Gradient A4, t_{R}=6.39 min, [M+H]⁺ 354;

¹H NMR (CDCl₃, ppm): δ 7.29-7.17 (2H, m), 6.93-6.84 (2H, m), 5.00 (1H, bs), 4.15-4.08 (3H, bm), 3.98-3.85 (5H, m), 3.64 (1H, bs), 3.28 (1H, bd), 3.10 (2H, m), 1.45 (9H, s).

### N. Standard Procedure for the Synthesis of Tether T143

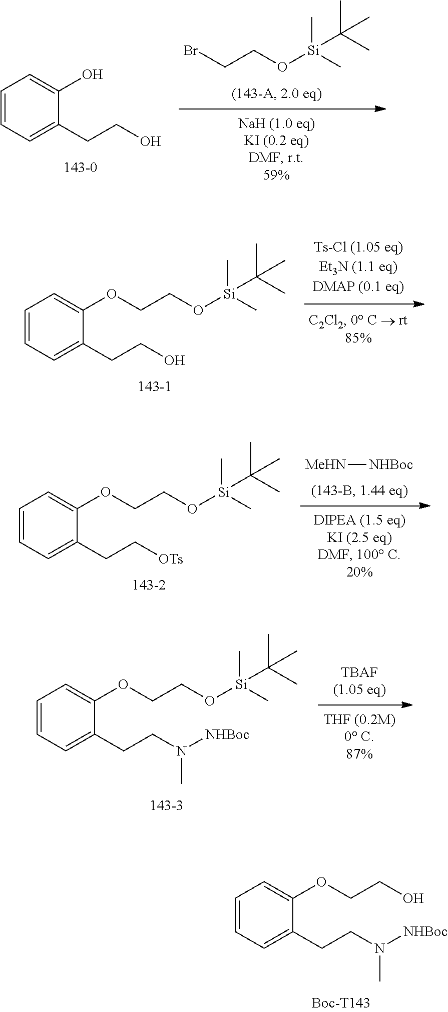

Step T143-1. NaH (60% in mineral oil, 2.32 g, 58 mmol, 1.0 eq) was added portion-wise to a well-stirred solution of 2-hydroxyphenethyl alcohol (143-0, Aldrich, 8.0 g, 58 mmol, 1.0 eq) in DMF (200 mL) at 0° C. under a nitrogen atmosphere. Stirring was continued for 10 min at 0° C., then the bromoalkane (143-A, 20.8 g. 87 mmol, 1.5 eq) added, followed by KI (1.9 g, 11.6 mmol, 0.2 eq), and the reaction stirred overnight allowing it to warm gradually to rt. HPLC can be used to monitor disappearance of the alcohol starting material. The solution was concentrated in vacuo (vacuum pump, bath T ca. 50° C.), then EtOAc (300 mL) added. The organic phase was washed with saturated aqueous NaHCO₃ (2×100 mL), water (1×100 mL), brine (1×100 mL), then dried (MgSO₄), filtered and the filtrate concentrated under reduced pressure. The resulting liquid residue was purified by flash chromatography (20% EtOAc/Hex) to yield 10.2 g (59%) of 143-1 as a slightly yellow liquid. This reaction was also performed from 863 μL of alcohol to afford 1.70 g of product (83%). The alkylation was also performed with K₂CO₃ as a base and heating at 70° C. to give 143-B1 in 57% yield.

TLC: R_{f}=0.29 (20% EtOAc/Hex; detection: UV, KMnO₄);

HPLC/MS: Gradient A4, t_{R}=9.50 min, [M+H]⁺ 297.

Step T143-2. Tosyl chloride (7.61 g, 39.9 mmol, 1.05 eq) was added portion-wise to a stirred solution of 143-1 (11.3 g, 38.0 mmol, 1.0 eq), DMAP (464 mg, 3.8 mmol, 0.1 eq) and triethylamine (5.81 mL, 41.8 mmol, 1.1 eq) in dichloromethane (127 mL) at 0° C. under a nitrogen atmosphere. Stirring was continued for 2 h at 0° C. (during which some salts precipitated), then 1 h at rt. When TLC monitoring indicated that all 143-1 was exhausted, 100 mL of dichloromethane were added and the solution washed with saturated aqueous NaHCO3 (2×100 mL), water (1×100 mL), brine (1×100 mL), then dried (MgSO₄), filtered and the filtrate concentrated under reduced pressure. The liquid residue was purified by flash chromatography (20% EtOAc/Hex) to afford 14.6 g (85%) of 143-2 as a yellow syrup. This reaction was also performed from 100 mg of alcohol to provide 138 mg of product (91%).

TLC: R_{f}=0.35 (20% EtOAc/Hex; detection: UV, KMnO₄);

¹H-NMR (CDCl₃, 300 MHz): δ 0.06 (6H, s), 0.89 (9H, s), 2.42 (3H, s), 2.97 (3H, t, J=7.0), 3.85-3.95 (4H, stack), 4.12 (2H, t, J=7.0), 6.75-6.87 (2H, m), 7.04-7.09 (1H, m), 7.14-7.25 (3H, m), 7.63-7.69 (2H, m).

Step T143-3. 143-B (see synthesis following, 6.82 g, 46.7 mmol, 1.44 eq) was added in one portion to a solution of 143-2 (14.6 g, 32.4 mmol, 1.0 eq), KI (13.5 g, 81 mmol, 2.5 eq) and diisopropylethylamine (8.46 mL, 48.6 mmol, 1.5 eq) in DMF (65 mL). The resulting suspension was stirred in an Ace Tube (Ace Glass, Inc., 150 mL capacity) at rt for 30 min under vacuum to degas DMF. The screw cap (Teflon coating) was replaced and the reaction heated to 100° C. overnight with stirring (upon heating, the suspension becomes a solution), after which HPLC indicated disappearance of the tosylate. The solution was cooled (some salts precipitated at it) and saturated aqueous NaHCO₃ added (300 mL). This was extracted with EtOAc (3×100 mL) and the combined organic layer washed with brine (50 mL), dried (MgSO₄), filtered and the filtrate concentrated in vacuo (vacuum pump to remove residual DMF). Purification by flash chromatography (20% EtOAc/Hex) afforded 2.70 g (20%) of 143-3 as a yellow oil. This reaction was also performed from 138 mg of 143-2 to give 89 mg of product (68%).

TLC: R_{f}=0.35 (20% EtOAc/Hex; detection: UV, KMnO₄);

HPLC/MS: Gradient A4, t_{R}=8.09, 11.05 min (possible rotamers), [M+H]⁺ 425;

¹H NMR (CDCl₃, 300 MHz): δ 0.10 (6H, s), 0.91 (9H, s), 1.46 (9H, s), 2.17 (2H, s), 2.60 (2H, s), 2.85 (3H, s), 3.98-4.05 (4H, stack), 5.60-5.75 (1H, br s), 6.80-6.90 (2H, m), 7.13-7.19 (2H, m).

Step T143-4. TBAF (1M in THF, 7.0 mL, 7.0 mmol, 1.1 eq) was added dropwise to a stirred solution of 143-3 (2.70 g, 6.36 mmol, 1.0 eq) in THF (32 mL) at 0° C. Stirring was continued for 2 h at 0° C. at which time TLC indicated no remaining starting material. The solution was concentrated in vacuo (bath T, rt) and the resulting yellow oil purified by flash chromatography (gradient, 10%, 50%, 70% EtOAc/Hex) to yield 143-4 as a slightly yellow oil that solidifies upon refrigeration (1.72 g, 87%). This reaction was also performed from 89 mg of 143-3 to afford 61 mg of product (94%).

TLC: R_{f}=0.10 (20% EtOAc/Hex; detection: UV, KMnO₄);

HPLC/MS: Gradient A4, t_{R}=5.72 min, [M+H]⁺ 311;

¹H-NMR (CDCl₃, 300 MHz): δ 1.47 (9H, s), 2.63 (3H, br s), 2.80-2.95 (4H, stack), 3.09-3.25 (1H, br s), 3.95-4.03 (2H, br s), 4.64-4.10 (2H, m), 5.75-5.79 (1H, br s), 6.81-6.92 (2H, m), 7.12-7.21 (2H, m).

### O. Standard Procedure for the Synthesis of Reagent 143-B

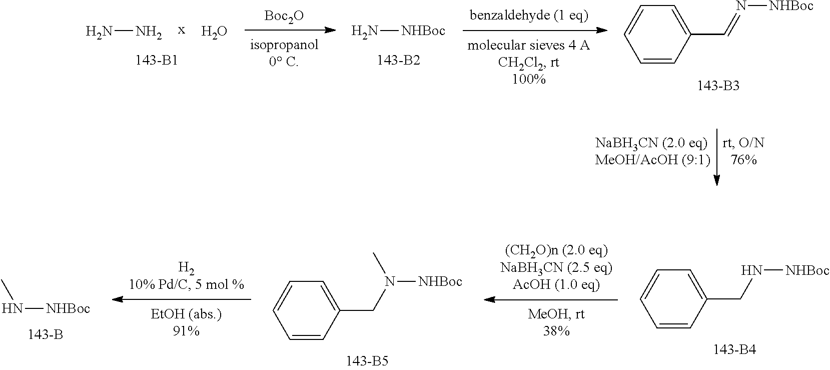

Step T143-5. Polyhydrated hydrazine (143-B1, Aldrich, contains an unknown amount of water; 47 g, approximately 734 mmol, 1.0 eq) was stirred in isopropanol (188 mL) at 0° C. for 15 min. Boc₂O (80 g, 367 mmol, 0.5 eq) in isopropanol (94 mL) was then added dropwise to the first solution at 0° C. The solution turned cloudy upon addition of this second solution and gas evolution was observed. This was stirred 20 min at 0° C., then concentrated in vacuo (bath T, 45° C.); the solution became clear upon heating. Dichloromethane (200 mL) was added to the residue and the solution dried over MgSO₄, filtered, and the filtrate concentrated in vacuo to provide 46.7 g of 143-B2 as a colorless syrup that solidified upon storage in the refrigerator. This was typically pure enough (TLC, ¹H NMR) to use in the next step. Flash chromatography (MeOH/dichloromethane) could also be performed to provide highly pure samples.

¹H-NMR (CDCl₃, 300 MHz): δ 1.41 (9H, s), 3.69 (2H, br s), 5.80 (1H, br s).

Step T143-6. Benzaldehyde (35.7 mL, 353 mmol, 1.0 eq) was added dropwise to a stirred suspension of 143-B2 (46.7 g, 353 mmol, 1.0 eq) and powdered 4 Å molecular sieves (Aldrich-activated, used as received, 9.3 g, 20% by weight) in dichloromethane (1 L) using a round-bottom flask fitted with a rubber septum. The reaction was monitored by NMR of removed aliquots and after 5 h showed completion. The sieves were removed by filtration and the filtrate concentrated in vacuo, with the product precipitating during evaporation, to afford 143-B3 as a white solid (78.1 g, quantitative) that was sufficiently pure to be used as such in the next reaction. TLC: R_{f}=0.70 (5% MeOH/CH₂Cl₂; detection: KMnO₄, UV).

Step T143-7. Sodium cyanoborohydride (44.4 g, 706 mmol, 2.0 eq) was added portion-wise to a stirred solution of 143-B3 (78.1 g, 353 mmol, 1.0 eq) in MeOH/AcOH (9/1, 1 L) at rt. The cloudy solution clears slowly upon addition of 143-B3 and was accompanied by H₂ evolution. The reaction was stirred overnight at rt (TLC and ¹H NMR showed completion). This was concentrated to dryness in vacuo (with at least one co-evaporation with toluene to remove AcOH) and the residue dissolved in saturated aqueous NaHCO₃ (900 mL). The aqueous layer was extracted with CH₂Cl₂ (3×300 mL) and the combined extracts were dried (MgSO₄), filtered, and the filtrate concentrated in vacuo to give 143-B4 as a colorless syrup (60.4 g, 76%) that was sufficiently pure by TLC and NMR to be used as such in the next step.

TLC: R_{f}=0.45 (2% MeOH/CH₂Cl₂; detection: KMnO4, UV);

¹H-NMR (CDCl₃, 300 MHz): δ 1.42 (9H, s), 3.98 (2H, s), 6.01 (1H, br s), 7.24-7.41 (5H, stack).

Step T143-8. Paraformaldehyde (27 g, 270 mmol, 2.0 eq), sodium cyanoborohydride (21 g, 337 mmol, 2.5 eq) and AcOH (7.73 mL, 135 mmol, 1.0 eq) were successively added to a stirred solution of 143-B4 (30 g, 135 mmol, 1.0 eq) in MeOH (450 mL) in a round-bottom flask fitted with a rubber septum at rt. The reaction was stirred overnight at rt at which time ¹H NMR of a removed aliquot showed a complete reaction (it was difficult to follow by TLC). This was concentrated in vacuo (bath T ca. 30° C.) to give a white gum that was dissolved in saturated aqueous NaHCO₃ (1 L). The aqueous layer was extracted with CH₂Cl₂ (3×500 mL), dried (MgSO₄), filtered, and the filtrate concentrated under reduced pressure to afford 12.1 g (38%) of 143-B5 as a white solid which was shown by NMR and TLC to be sufficiently pure to be used as such.

TLC: Rf=0.35 (2% MeOH/CH₂Cl₂; detection: KMnO₄, UV);

¹H NMR (CDCl₃, 300 MHz): δ 1.40 (9H, s), 2.61 (3H, s), 3.92 (2H, br s), 4.02 (1H, br s), 5.42 (1H, br s), 7.26-7.40 (5H, stack).

Step T143-9. Argon was bubbled thru a solution of 143-B5 (12.1 g, 51.3 mmol, 1.0 eq) in absolute ethanol (256 mL) at rt for 30 min. 10% Pd/C (2.72 g, 2.56 mmol, 0.05 eq) was then added carefully to the stirred solution and hydrogen bubbled through the mixture for 30 min. After this, a balloon of H₂ was fitted over the rubber septum-sealed round-bottom flask and the reaction stirred overnight at rt. Filtration through a pad of Celite, washing with 10% MeOH in CH₂Cl₂, followed by concentration of the filtrate in vacuo afforded 143-B (7.49 g, 91%) as a colorless oil that solidified upon standing. ¹H NMR and TLC showed that this material was pure enough to be used as obtained.

TLC: R_{f}=0.60 (2% MeOH/CH₂Cl₂; detection: KMnO₄, UV);

¹H-NMR (CDCl₃, 300 MHz): δ 1.41 (9H, s), 2.61 (3H, s), 6.01 (1H, br s).

### P. Standard Procedure for the Synthesis of Tether T144

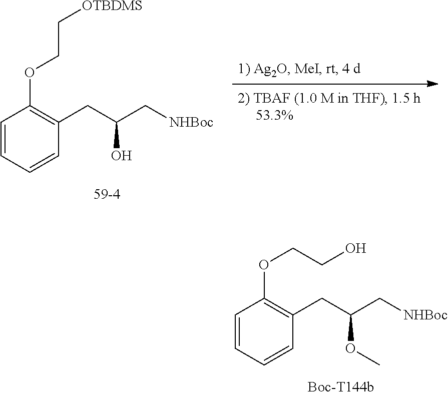

Step T144-1. To a solution of 59-4 (synthesized as described in the standard procedure for T59, 4.0 g, 9.4 mmol, 1.0 eq) in MeI (37.6 mL) was added Ag₂O (21.8 g, 94 mmol, 10 eq) and the reaction stirred 2 d at rt. The solids were removed by filtration and rinsed with MeI. To the filtrate was added a second portion of Ag₂O (21.8 g, 94 mmol, 10 eq) and the reaction stirred an additional 2 d. Monitoring of the reaction was done by TLC (3/7, EtOAc/Hex). The solution was filtered and the residue rinsed with DCM. The filtrate was concentrated in vacuo and the crude residue purified by flash chromatography (gradient, 20% to 25% EtOAc/Hex) to give the protected methyl ether intermediate (2.2 g, 53.3%). In addition, some starting material was recovered (1.6 g).

HPLC/MS: Gradient A4, t_{R}=13.54 min, [M+H]⁺ 440.

Step T144-2. To a solution of the protected methyl ether intermediate (2.2 g, 5.0 mmol, 1.0 eq) in THF (20 mL) was added a solution 1.0 M TBAF in THF (7.5 mL, 7.5 mmol, 1.5 eq) and the reaction stirred 1.5 h at it Brine was added and the aqueous phase extracted with MTBE (3×). The combined organic phase was dried over MgSO₄, filtered and the filtrate concentrated to dryness in vacuo. The residue was purified by flash chromatography (gradient, 1/1 to 3/2 EtOAc/Hex) to provide Boc-T144b (1.6 g, 100%).

HPLC/MS: Gradient A4, t_{R}=6.43 min, [M+H]⁺ 326;

¹H NMR (CDCl₃, ppm): δ 7.22-7.16 (2H, m), 6.93-6.83 (2H, m), 5.05 (1H, bs), 4.16-4.07 (3H, m), 4.00-3.98 (2H, m), 3.59 (1H, bs), 3.33 (3H, s), 3.06-2.9 (1H, m), 2.90-2.79 (2H, m), 1.44 (9H, s).

The enantiomeric tether, Boc-T144a, can be accessed from the enantiomeric precursor 59-5. As previously indicated, this compound is in turn synthesized as described for 59-4, but using AD-mix α.

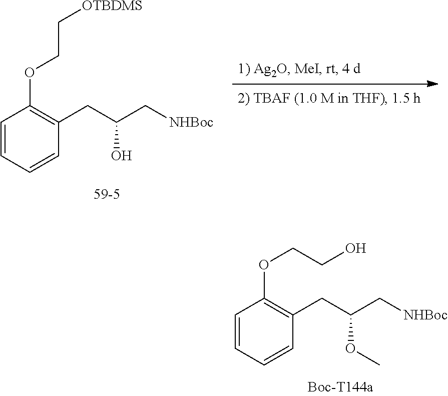

### Q. Standard Procedure for the Synthesis of Tether T145

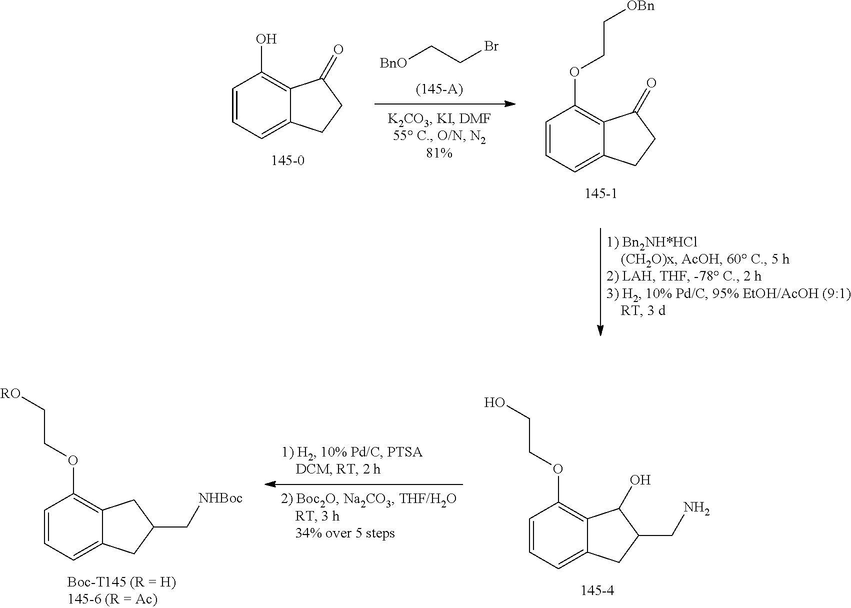

Step T145-1. To a solution of 7-hydroxyindanone (145-0, 2.0 g, 13.5 mmol, 1.0 eq) and benzyl 2-bromoethyl ether (145-A, 3.16 mL, 20.3 mmol, 1.5 eq) in DMF (Drisolv, 50 mL) were added potassium carbonate (2.33 g, 16.9 mmol, 1.25 eq) and potassium iodide (448 mg, 2.70 mmol, 0.20 eq). The solution was heated to 55° C. and stirred overnight under nitrogen. The reaction was diluted with water (200 mL) and the mixture extracted with ethyl acetate (3×50 mL). The organic phases were combined, dried with magnesium sulfate, filtered, and the filtrate evaporated to dryness under reduced pressure. The residue was purified by flash chromatography (30% EtOAc/Hex) to give 145-1 (3.08, 81%) as a white solid.

Step T145-2. Dibenzylamine (2.6 mL, 13.6 mmol, 1.25 eq) was dissolved in methanol (30 mL), then hydrochloric acid (4 M in dioxane, 5 mL, 20 mmol, 16 eq) added. The mixture was concentrated under reduced pressure to give dibenzylamine hydrochloride. This material was dissolved in acetic acid (40 mL), 145-1 (3.08 g, 10.9 mmol, 1.0 eq) and paraformaldehyde (425 mg, 14.2 mmol, 1.3 eq) added, and the mixture stirred at 60° C. for 5 h. The reaction was concentrated under reduced pressure, then DCM (50 mL) added and the mixture treated with a saturated aqueous solution of sodium bicarbonate until a pH of 9 was attained. The aqueous layer was discarded and the organic layer dried over magnesium sulfate, filtered, and the filtrate concentrated under reduced pressure. The residue was purified by flash chromatography (10% MTBE/toluene) to give 145-2 as a yellowish oil. Although this material contained dibenzylamine, it was suitable for use in the next step.

HPLC/MS: Special conditions, t_{R}=5.63 min, [M+H]⁺ 492.

Step T145-3. 145-2 (4.47 g, 9.10 mmol, 1.0 eq) was dissolved in THF (75 mL), cooled to −78° C., then treated with LAH (0.175 g, 4.55 mmol, 0.5 eq) for 2 h. At that time, a 20% aqueous solution of potassium hydroxide (50 mL) was added and the mixture extracted with ethyl acetate (3×). The combined organic phase was dried over magnesium sulfate, filtered, and the filtrate concentrated under reduced pressure to give 145-3. Since the product and the starting material are not distinguishable by TLC or HPLC analysis, MS analysis must be checked for completion of the reaction.

HPLC/MS: Special conditions, t_{R}=5.70 min, [M+H]⁺ 494.

Step T145-4. 145-3 (3.78 g) from the previous step was dissolved in a mixture of 95% ethanol and acetic acid (100 mL, 9:1). Palladium on charcoal (3.78 g, 10% w/w, 50% wet) and the mixture submitted to 1 atmosphere of hydrogen gas (atmospheric pressure). After 3 d, the mixture was filtered through Celite and the filter cake washed with acetic acid and 95% ethanol. The solvent was removed under reduced pressure with low heat (bath T≦40° C.) to obtain 145-4.

HPLC/MS: Special conditions, t_{R}=2.34 min, [M+H]⁺ 224.

Step T145-5. 145-4 as obtained from the previous step was dissolved in DCM (80 mL), palladium on charcoal (500 mg, 10% w/w, 50% wet) and p-toluene sulfonic acid (2.9 g, 15.34 mmol, 2 eq) added and the mixture submitted to 1 atmosphere of hydrogen gas (atmospheric pressure). After 2 h, the mixture was filtered through Celite and the filter cake washed with a mixture of THF and water (200 mL, 1:1). Sodium carbonate (4.3 g, 40.1 mmol, 5.3 eq) was added and the organic solvents were removed under reduced pressure to leave an aqueous solution of the amino acid 145-5. Disappearance of the starting material was determined by HPLC analysis.

HPLC/MS: Special conditions, t_{R}=2.95 min, [M+H]⁺ 208.

Step T145-6. To the aqueous solution of 145-4 were added THF (100 mL) and Boc₂O (2.5 g, 11.5 mmol, 1.5 eq). The mixture was stirred for 3 h, then diluted with a saturated aqueous ammonium chloride solution (400 mL). The aqueous phase was extracted with ethyl acetate (3×100 mL). The combined organic layer washed with brine (50 mL), dried over magnesium sulfate, filtered, and the filtrate concentrated to dryness under reduced pressure. The residue was purified by flash chromatography (40% EtOAc/hexanes) to give Boc-T145 as a colorless oil (1.03 g, 34% overall yield for 5 steps) along with the corresponding acetate of the tether alcohol (145-6, 600 mg, 17% overall yield for 5 steps).

HPLC/MS: Special conditions, t_{R}=5.57 min, [M+H]⁺ 308.

¹H NMR (CDCl₃, 300 MHz): δ 7.11 (t, 1H, J=8.0 Hz, CH aryl), 6.83 (d, 1H, J=7.0 Hz, CH aryl), 6.66 (d, 1H, d=8.0 Hz, CH aryl), 4.67 (bs, 1H, NHBoc), 4.12-4.08 (m, 2H, CH₂O), 3.98-3.93 (m, 2H, CH₂O), 3.23-3.18 (m, 1H, CHNHBoc), 3.11-2.99 (m, 2H, arylCH₂), 2.75-2.58 (m, 3H, CH₂CHCH₂), 1.45 (s, 9H, C(CH₃)₃)

### R. Standard Procedure for the Synthesis of Tether T146

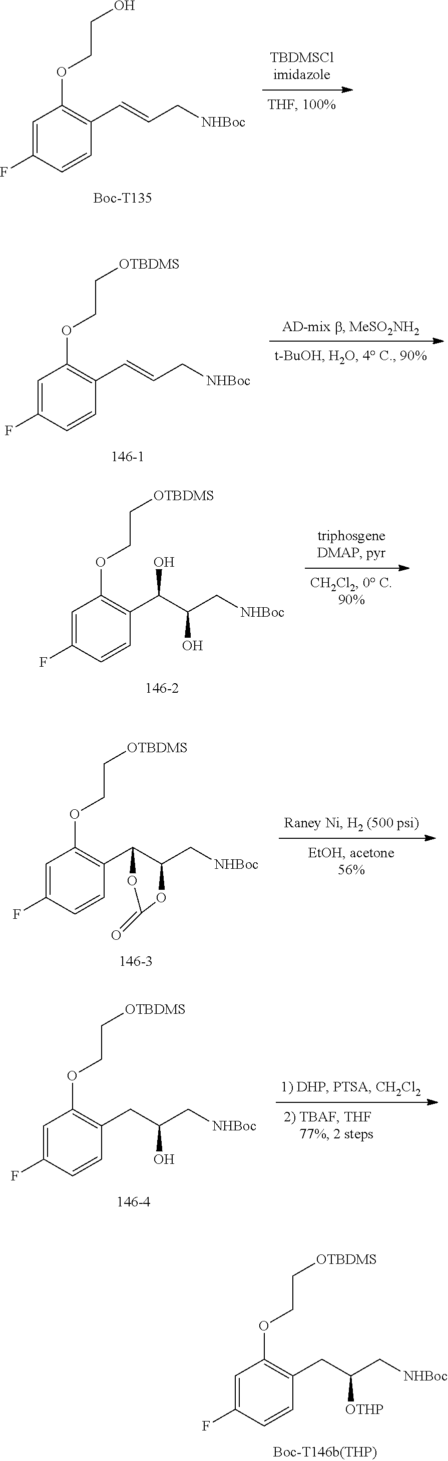

Step T146-1: To a solution of Boc-T135 (3.5 g, 11.0 mmol, 1.0 eq) in THF (50 mL) were added imidazole (1.5 g, 22.0 mmol, 2.0 eq) and TBDMSCl (2.21 g, 15.0 mmol, 1.3 eq) and the mixture stirred 2 h with monitoring by TLC. The solution was then treated with saturated aqueous NH₄Cl and the aqueous phase extracted with EtOAc (2×). The combined organic phase was dried over MgSO₄, filtered and the filtrate concentrated under reduced pressure. The resulting residue was filtered through a silica gel pad (10% EtOAc/90% hexanes) to give 146-1 as a white solid (100%).

TLC: R_{f}=0.60 (30% EtOAc/70% hexanes; detection: UV, Mo/Ce)

HPLC/MS: Gradient A4, t_{R}=13.51 min, [M]⁺ 425

Step T146-2: To a solution of 146-1 (4.46 g, 10.5 mmol, 1.0 eq) in a mixture of H₂O:t-BuOH (1:1, 104 mL) were added AD-mix 13 (12.8 g) and methanesulfonamide (998 mg, 10.5 mmol, 1.0 eq) and the resulting orange mixture stirred at 4° C. for 36-48 h during which time the color changes to yellow. Once TLC indicated the reaction was complete, sodium sulfite (15 g, 12.0 eq) was added and the mixture stirred at room temperature 1 h. The mixture was extracted with EtOAc (3×), then the combined organic phase extracted with water and brine. The organic phase was dried over MgSO₄, filtered and the filtrate concentrated under reduced pressure. The residue was purified by flash chromatography (50% EtOAc/50% hexanes) to give 146-2 as a yellow oil (96%).

TLC: R_{f}=0.41 (50% EtOAc/50% hexanes; detection: UV, KMnO₄)

HPLC/MS: Gradient A4, t_{R}=10.63 min, [M]⁺ 459, [M+Na]⁺ 482

Step T146-3: To a solution of 146-2 (4.5 g, 9.79 mmol, 1.0 eq) in DCM (62 mL) at 0° C. were added pyridine (3.1 mL) and DMAP (60 mg, 0.49 mmol, 0.05 eq). Triphosgene (2.9 g, 9.79 mmol, 1.0 eq) in DCM (10 mL) was then slowly added to this mixture. The reaction was stirred at 0° C. for 45 min at which time TLC indicated the reaction was completed. The solution was treated with saturated aqueous NH₄Cl and the organic phase separated. The aqueous phase was extracted with Et₂O (2×) and the combined organic phase extracted with saturated aqueous NH₄Cl. The organic phase was dried over MgSO₄, filtered and the filtrate concentrated under reduced pressure. The resulting residue was filtered through a silica gel pad (30% EtOAc/70% hexanes) to give 146-3 as a yellow oil (91%).

TLC: R_{f}=0.56 (50% EtOAc/50% hexanes; detection: UV, Mo/Ce)

HPLC/MS: Gradient A4, t_{R}=11.96 min, [M]⁺ 485

Step T146-4: To a solution of 146-3 (2.49 g, 4.9 mmol, 1.0 eq) in a mixture of 95% EtOH:acetone (3:1, 60 mL) was added Raney Ni (50% in water, 16 mL, 49 mmol, 10.0 eq). The reaction was stirred under 500 psi of hydrogen in a Parr hydrogenator for one week. At that time, N₂ was bubbled through the mixture to remove excess hydrogen, then the mixture filtered though a Celite pad and rinsed with EtOAc. Concentration of the filtrate under reduced pressure and flash chromatography (20% EtOAc/80% Hex) of the residue provided 146-4 as a colorless oil (1.1 g, 56%).

TLC: R_{f}=0.29 (30% EtOAc/70% hexanes; detection: UV, Mo/Ce)

HPLC/MS: Gradient A4, t_{R}=12.35 min, [M+H]⁺ 444

Step T146-5: To a solution of the alcohol 146-4 (1.1 g, 2.48 mmol, 1.0 eq) in CH₂Cl₂ (16 mL) were added DHP (272 μL, 2.97 mmol, 1.2 eq) and PTSA (24 mg, 0.124 mmol, 0.05 eq). The mixture was stirred at room temperature for 1 h with TLC monitoring (30% EtOAc/70% hexanes; detection: UV, Mo/Ce; R_{f}=0.51). Additional DHP (2×0.3 eq) was added to force the reaction to completion. At that time, the solution was treated with saturated aqueous NaHCO₃, then the aqueous phase extracted with CH₂Cl₂. The combined organic phase was dried over MgSO₄, filtered and the filtrate concentrated under reduced pressure. The crude residue was purified by flash chromatography (20% EtOAc/80% Hex) to give 1.2 g of the intermediate diprotected diol.

The residue was dissolved in THF (16 mL) and a 1 M solution of TBAF in THF (4.96 mL, 4.96 mmol, 2.0 eq) added. The mixture was stirred at rt for 1 h. When TLC indicated the reaction was complete, the mixture was treated with brine, the layers separated, and the aqueous phase extracted with EtOAc. The combined organic phase was dried over MgSO₄, filtered and the filtrate concentrated to dryness under reduced pressure. The residue was purified by flash chromatography (50% EtOAc/50% hexanes) to give Boc-T146b(THP) as a yellow oil (76%, 3 steps).

TLC: R_{f}=0.12 (30% EtOAc/70% hexanes; detection: UV, Mo/Ce)

HPLC/MS: Gradient A4, t_{R}=7.49 min, [M]⁺ 413, [M+Na]⁺ 436

To obtain Boc-T146a and its THP-protected derivative, the same procedure as above can be followed, but utilizing AD-mix α. Other suitable protecting groups in place of THP can be introduced in the last step as well.

### S. Standard Procedure for the Synthesis of Tether T147

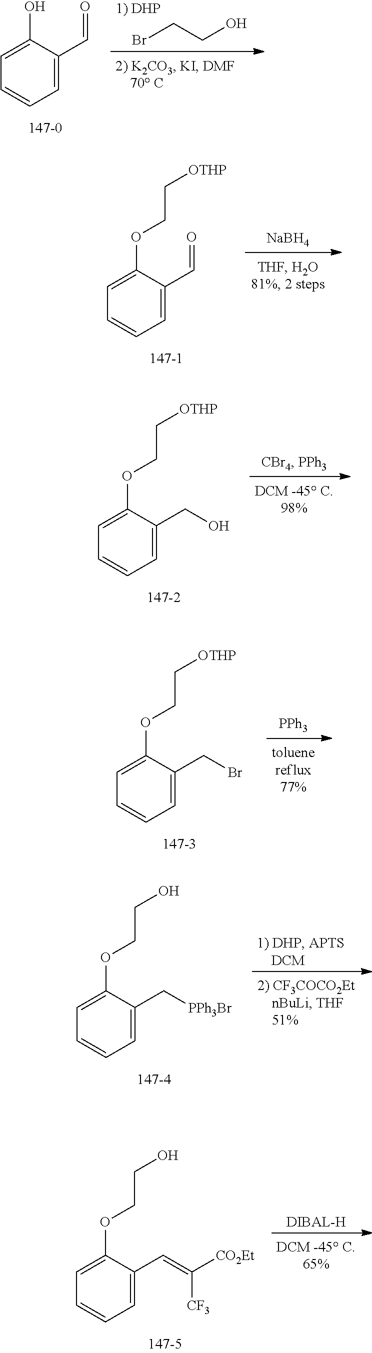

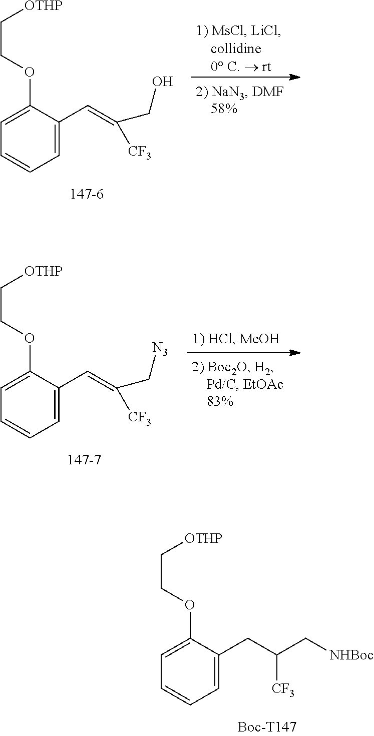

Step T147-1. Dihydropyran (13.4 mL, 146 mmol, 1.5 eq) was added dropwise at 0° C. to 2-bromoethanol (10.3 mL, 146 mmol, 1.5 eq). The mixture was stirred 30 min at 0° C. and then 2 h at rt. Salicylaldehyde (147-0, 10.2 mL, 97.0 mmol, 1.0 eq) was added to this mixture, followed by potassium carbonate (14.6 g, 106 mmol, 1.1 eq), potassium iodide (3.15 g, 19 mmol, 0.2 eq) and dry DMF (50 mL). The reaction was stirred at 70° C. overnight. The solution was cooled to rt and diluted with ethyl ether (200 mL). The inorganic salts were removed by filtration and the filtrate diluted with hexanes (200 mL). The organic layer was washed with water (3×), then concentrated to dryness under reduced pressure. Compound 147-1 thus obtained was reduced directly in the next step without further purification.

TLC: R_{f}=0.18 (MTBE/Hexanes, 1/4; detection: UV, vanillin)

HPLC/MS: Gradient A4, t_{R}=6.27 min, [M]⁺ 250, [M+Na]⁺ 273

Step T147-2. Crude compound 147-1 was dissolved in THF (200 mL) and water (200 mL) and cooled at 0° C. To this mixture, sodium borohydride (3.67 g, 97 mmol) was added and the reaction followed by TLC (20% EtOAc/Hexanes). When no more 147-1 was present, water (400 mL) was added and the mixture extracted with ethyl acetate (3×100 mL). The combined organic layer was washed with brine, dried over magnesium sulfate, filtered, and the filtrate concentrated under reduced pressure. The material obtained was purified by flash chromatography (40% EtOAc/Hexanes) to obtain 147-2 as a colorless oil (19.7 g, 81% over two steps).

TLC: R_{f}=0.08 (20% EtOAc/Hexanes; detection: UV, vanillin)

HPLC/MS: Gradient A4, t_{R}=5.79 min, [M]⁺ 252, [M+Na]⁺ 275

Step T147-3. 147-2 (17.9 g, 71 mmol, 1.0 eq) and carbon tetrabromide (23.6 g, 71 mmol, 1.0 eq) were dissolved in DCM (500 mL) and the solution cooled to −45° C. using an ethylene glycol/water/dry ice bath. Triphenylphosphine (18.6 g, 71 mmol, 1.0 eq) was added to this portion-wise, waiting for all the triphenylphosphine to dissolve before each subsequent addition. The mixture was stirred 45 min and concentrated under reduced pressure. The residue was purified by flash chromatography (MTBE/DCM, 1/19) to provide 147-3 as a yellowish oil (21.9 g, 98%).

TLC: R_{f}=0.68 (MTBE/DCM, 1/9; detection: UV, vanillin)

HPLC/MS: Gradient A4, t_{R}=7.51 min, [M+H]⁺ 315, [M+Na]⁺ 337, 339

Step T147-4. Triphenylphosphine (13.0 g, 49.4 mmol, 1.0 eq) was added to a solution of 147-3 (15.6 g, 49.4 mmol, 1.0 eq) in toluene (300 The mixture was refluxed for 4 h, then cooled to rt. The precipitated solid was removed by filtration through a fine fritted glass filter and the solid obtained dried under vacuum (oil pump) for 1 h. The phosphonium salt 147-4 was obtained as a white solid (18.7 g, 77%). Note that the THP moiety was removed in this process as evidenced by both ¹H NMR in CDCl₃ and HPLC. This had to be replaced before the next transformation as described in the next step.

HPLC/MS: Gradient A4, t_{R}=5.72 min, [M]⁺ 413

Step T147-5. APTS (8 mg, 0.02 mmol, 0.001 eq) was added to a solution of 147-4 (18.6 g, 37.6 mmol, 1.0 eq) and DHP (17.2 mL, 188 mmol, 5.0 eq) in DCM (200 mL). The mixture was stirred 1 h at rt, then the solvent removed under reduce pressure. The residue was placed under vacuum (oil pump) to obtain a foam. Dry THF (Drisolv, new bottle, 400 mL) was added and the suspension stirred at rt. BuLi (1.6 M in hexane's, 25.1 mL, 37.6 mmol, 1.0 eq) was added and the mixture stirred for 30 min. Ethyl trifluoropyruvate (5.00 mL, 37.6 mmol, 1.0 eq) was then added and the reaction stirred for 10 min. The mixture was poured into water (1.4 L) and extracted with MTBE (4×200 mL). The combined organic layer was dried over magnesium sulfate, filtered, and the filtrate concentrated under reduced pressure. The residue was purified by flash chromatography (30% EtOac/Hexanes) to yield 147-5 as a colorless oil (7.47 g, 51%).

TLC: R_{f}=0.53 (40% EtOAc/Hexanes; detection: UV, vanillin)

HPLC: Gradient A4, t_{R}=6.58 min (note that some cleavage of the THP protecting group was observed)

Step T147-6. Ester 147-5 (7.47 g, 19.3 mmol, 1.0 eq) was dissolved in DCM (Drisolv, 200 mL) and the solution cooled to −45° C. using an ethylene glycol/water/dry ice bath. DIBAL-H (1 M in DCM, 58 mL, 58 mmol, 3.0 eq) was added to the solution. The reaction was monitored by TLC (30% MTBE/Hexanes) and the temperature of the reaction allowed to increase slowly until completion of the reaction was observed. Potassium hydroxide (20% w/v aqueous, 300 mL) was added and the mixture extracted with DCM (3×100 mL). The combined organic layer was dried over magnesium sulfate, filtered, and the filtrate concentrated under reduced pressure. The crude product was purified by flash chromatography (MTBE/hexanes, 3/7) to give 147-6 as a colorless oil (4.33 g, 65%).

TLC: R_{f}=0.11 (MTBE/Hexanes, 1/4; detection: UV, vanillin)

HPLC/MS: Gradient A4, t_{R}=7.01 min, [M]⁺ 346, [M+Na]⁺ 369

Step T147-7. Lithium chloride (583 mg, 13.8 mmol, 1.1 eq) was dissolved in dry DMF (30 mL) at rt, then 147-6 (4.33 g, 12.5 mmol, 1.0 eq) and 2,4,6-collidine (1.91 mL, 14.4 mmol, 1.15 eq) were added and the mixture cooled to 0° C. Methanesulfonyl chloride (freshly distilled improves the yield, 1.12 mL, 14.4 mmol, 1.15 eq) was added and the mixture warmed to rt and stirred for 2 h. Sodium azide (4.07 g, 62.6 mmol, 5.0 eq) was added and the mixture stirred overnight. The reaction was diluted with water (400 mL) and extracted with MTBE (3×). The combined organic layer was washed with saturated sodium bicarbonate, water and brine, dried over magnesium sulfate; filtered, and the filtrate concentrated under reduced pressure. The residue was purified by flash chromatography (30% MTBE/hexanes). 147-7 was obtained as a colorless oil (2.70 g, 58%).

TLC: R_{f}=0.34 (MTBE/Hexanes, 3/7; detection: UV, vanillin)

HPLC/MS: Gradient A4, t_{R}=10.22 min, [M−N₂]⁺ 343

Step T147-8. The azide 147-7 (834 mg, 2.25 mmol, 1.0 eq) was dissolved in methanol (25 mL). Concentrated HCl (0.25 mL) was added and the reaction monitored by TLC (30% MTBE/hexanes). When the reaction was complete by TLC, the reaction was concentrated under reduced pressure, then dried under vacuum (oil pump). The deprotected material (635 mg, 98%) was dissolved in ethyl acetate (10 mL), then Boc₂O (725 mg, 3.32 mmol, 1.5 eq) and Pd/C (10% w/w, 50% wet, 65 mg) added and the mixture hydrogenated under 50 psi of hydrogen for 24 h. The reaction was filtered through Celite, washed with ethyl acetate, and the combined filtrate and washings concentrated under reduced pressure. The residue was purified by flash chromatography (40% EtOAc/hexanes). Boc-T147 was obtained as colorless oil (668 mg, 83%).

TLC: R_{f}=0.41 (MTBE/Hexanes, 2/3; detection: UV, ninhydrin)

HPLC/MS: Gradient A4, t_{R}=7.16 min, [M+Na]⁺ 386

¹H NMR (300 MHz, DMSO-d₆): δ 7.21-7.17 (m, 2H, Ar), 6.90-6.80 (m, 3H, Ar+NHBoc), 4.82 (t, 1H, J=5.4 Hz, OH), 4.00 (t, 2H, J=5.1 Hz, ArOCH₂), 3.73 (q, 2H, J=5.4 Hz, CH₂OH), 3.22-3.00 (m, 2H, CH₂NHBoc), 2.85-2.62 (m, 3H, CH₂Ar+CHCF₃), 1.35 (s, 9H, C(CH₃)₃).

### T. Standard Procedure for the Synthesis of Tether T148

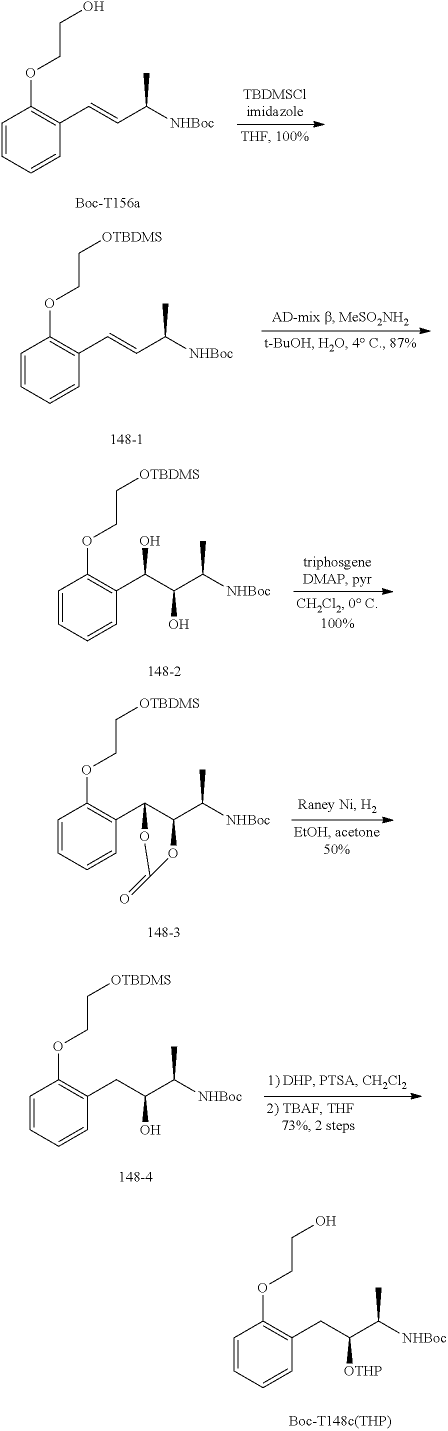

Step T148-1: To a solution of Boc-T156a (2.57 g, 8.36 mmol, 1.0 eq) in THF (42 mL) were added imidazole (1.14 g, 16.7 mmol, 2.0 eq) and TBDMSCl (1.64 g, 10.9 mmol, 1.3 eq) and the mixture stirred 2 h with monitoring by TLC. The solution was then treated with saturated aqueous NH₄Cl and the aqueous phase extracted with EtOAc (3×). The combined organic phase was dried over MgSO₄, filtered and the filtrate concentrated under reduced pressure. The resulting residue was purified by flash chromatography (15% EtOAc/85% hexanes) to give 148-1 as a colorless oil (100%).

TLC: R_{f}=0.54 (25% EtOAc/75% hexanes; detection: UV, vanillin)

HPLC/MS: Gradient A4, t_{R}=13.72 min, [M]⁺ 421, [M+Na]⁺ 444

Step T148-2: To a solution of 148-1 (2.80 g, 6.60 mmol, 1.0 eq) in a mixture of H₂O:t-BuOH (1:1, 66 mL) were added AD-mix β (8.1 g) and methanesulfonamide (632 mg, 6.60 mmol, 1.0 eq) and the resulting orange mixture stirred at 4° C. for 4 d. Once TLC indicated the reaction was complete, sodium sulfite (15.8 g, 125.4 mmol, 19.0 eq) was added and the mixture stirred at room temperature 1 h. Water was added and the mixture extracted with EtOAc (3×), then the combined organic phase extracted with water and brine. The organic phase was dried over MgSO₄, filtered and the filtrate concentrated under reduced pressure. The residue was purified by flash chromatography (gradient, 30% to 50% EtOAc/hexanes) to give 148-2 as a colorless oil (2.60 g, 87%).

TLC: R_{f}=0.32 (30% EtOAc/70% hexanes; detection: UV, vanillin)

HPLC/MS: Gradient A4, t_{R}=11.25 min, [M+H]⁺ 456

Step T148-3: To a solution of 148-2 (2.6 g, 5.7 mmol, 1.0 eq) in DCM (30 mL) at 0° C. were added pyridine (2.0 mL) and DMAP (35 mg, 0.29 mmol, 0.05 eq). Triphosgene (1.7 g, 5.7 mmol, 1.0 eq) in DCM (5 mL) was then slowly added to this mixture. The reaction was stirred at 0° C. for 1 h at which time TLC indicated the reaction was completed. The solution was treated with saturated aqueous NH₄Cl and the organic phase separated. The aqueous phase was extracted with DCM (3×). The combined organic phase was dried over MgSO₄, filtered and the filtrate concentrated under reduced pressure. The resulting residue was filtered through a silica gel pad (30% EtOAc/70% hexanes) to give 148-3 as a yellow oil (2.7 g, 100%).

TLC: R_{f}=0.53 (30% EtOAc/70% hexanes; detection: UV, vanillin)

HPLC/MS: Gradient A4, t_{R}=12.00 min, [M]⁺ 481

Step T148-4: To a solution of 148-3 (3.1 g, 6.4 mmol, 1.0 eq) in a mixture of 95% EtOH:acetone (3:1, 80 mL) was added Raney Ni (50% in water, 7.5 mL, 64.0 mmol, 10.0 eq). Hydrogen was bubbled into the solution for 2 d. At that time, N₂ was bubbled through the mixture to remove excess hydrogen, then the mixture filtered though a Celite pad and rinsed with EtOAc. Concentration of the filtrate under reduced pressure and flash chromatography (gradient 20% to 25% EtOAc/Hex) of the residue provided 148-4 as a colorless oil (1.4 g, 50%).

TLC: R_{f}=0.44 (30% EtOAc/70% hexanes; detection: UV, vanillin)

HPLC/MS: Gradient A4, t_{R}=12.69 min, [M+H]⁺ 440

Step T148-5: To a solution of the alcohol 148-4 (1.4 g, 3.2 mmol, 1.0 eq) in CH₂Cl₂ (30 mL) were added DHP (0.35 mL, 3.8 mmol, 1.2 eq) and PTSA (30 mg, 0.16 mmol, 0.05 eq). The mixture was stirred at room temperature for 2 h with TLC monitoring (30% EtOAc/70% hexanes; detection: UV, vanillin; R_{f}=0.54). At that time, the solution was treated with saturated aqueous NaHCO₃, then the aqueous phase extracted with CH₂Cl₂ (3×). The combined organic phase was dried over MgSO₄, filtered and the filtrate concentrated under reduced pressure. The residue was sufficiently pure to continue on to the next step. The residue was dissolved in THF (30 mL) and a 1 M solution of TBAF in THF (4.8 mL, 4.8 mmol, 2.0 eq) added. The mixture was stirred at it for 1 h. When TLC indicated the reaction was complete, the mixture was treated with brine, the layers separated, and the aqueous phase extracted with EtOAc (3×). The combined organic phase was dried over MgSO₄, filtered and the filtrate concentrated to dryness under reduced pressure. The residue was purified by flash chromatography (gradient, 30% to 50% EtOAc/hexanes) to give Boc-T148c(THP) as a yellow oil (73%, 2 steps).

TLC: R_{f}=0.16 (30% EtOAc/70% hexanes; detection: UV, vanillin)

HPLC/MS: Gradient A4, t_{R}=8.11 min, [M]⁺ 409, [M+Na]⁺ 432

To obtain Boc-T148a and its THP-protected derivative, the same procedure as described above can be followed, but utilizing AD-mix α. Other suitable protecting groups in place of THP can be introduced in the last step as well. Similarly, starting from T156b, and using the same procedures as above utilizing AD-mix-β and AD-mix-α, provide the diastereomeric tethers Boc-T148d and Boc-T148b, respectively. Appropriate protection of the hydroxyl moiety for these tethers, including THP, can be done using standard techniques.

### U. Standard Procedure for the Synthesis of Tether T149

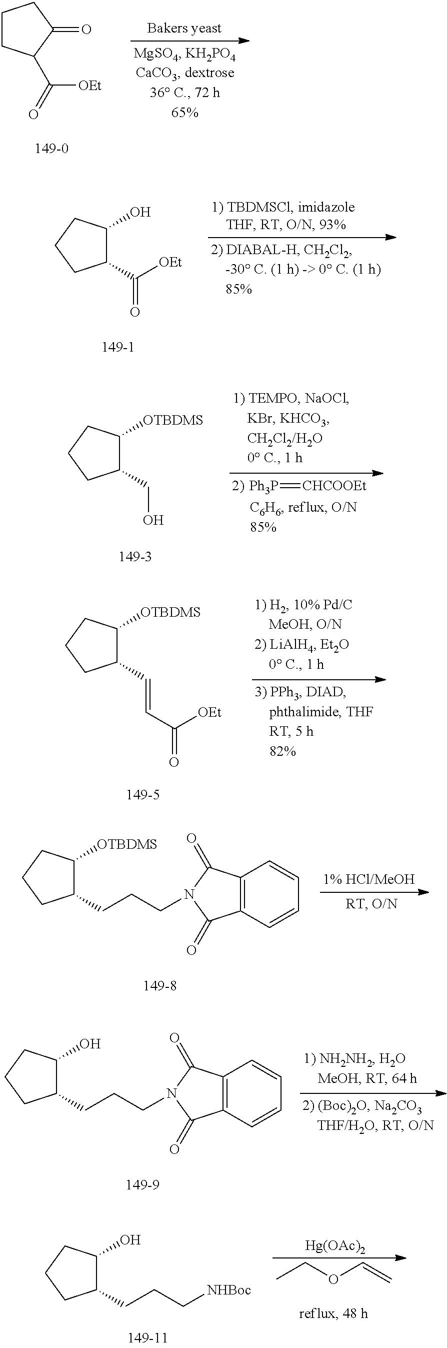

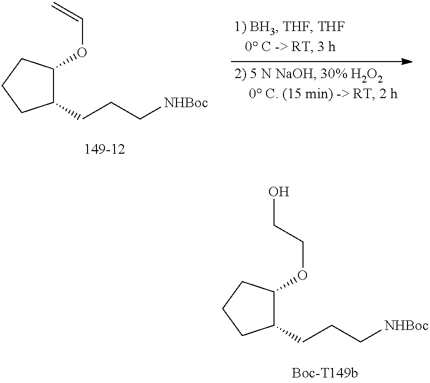

Boc-T149b was synthesized using an almost identical procedure to that already described for the corresponding cyclohexyl derivative, Boc-T104b. However, the starting chiral β-hydroxyester, T149-1, was accessed through asymmetric reduction of the β-ketoester, 149-0, using Baker's yeast as described below.

Step 149-1. (Adapted from the procedure in Crisp, G. T.; Meyer, A. G. Tetrahedron. 1995, 51, 5831-5845.) MgSO₄ (2 g), KH₂PO₄ (8 g) CaCO₁ (10 g) and dextrose (304 g) were added to water (2 L) at 36° C. Baker's yeast (24 g) was added and the mixture stirred using a mechanical stirrer due to the thickness of the solution at 36° C. for 45 min. The β-keto-ester 149-0 (20.3 g, 130 mmol) was slowly added over approximately 5 min to the mixture and the reaction stirred 72 h at 36° C. The mixture was filtered trough a Celite pad which was rinsed with water (2×300 mL). The combined filtrate and washings were extracted with Et₂O (5×500 mL) and the combined organic phase washed with brine, dried over MgSO₄, filtered, and the filtrate concentrated under reduced pressure. The residue was purified by vacuum fractional distillation (b.p 40° C., oil pump) to give 149-1 as a colorless oil (13.3 g, 65%). Compound 149-1 is also commercially available (Julich, now Codexis, product no. 31.60).

HPLC/MS: Gradient A4, t_{R}=4.11 min, [M+H]⁺ 159.

### V. Standard Procedure for the Synthesis of Tethers T150a and T150b

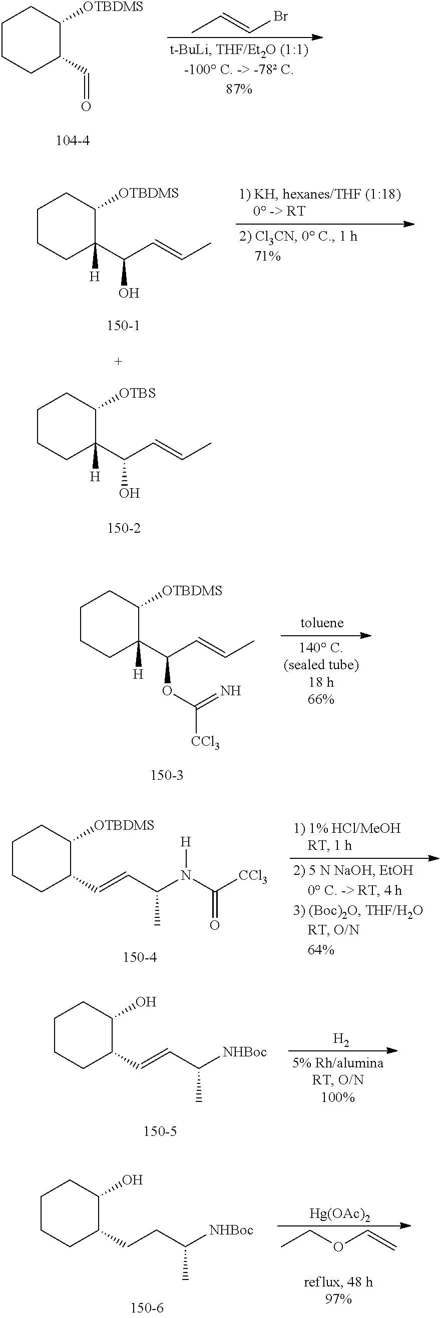

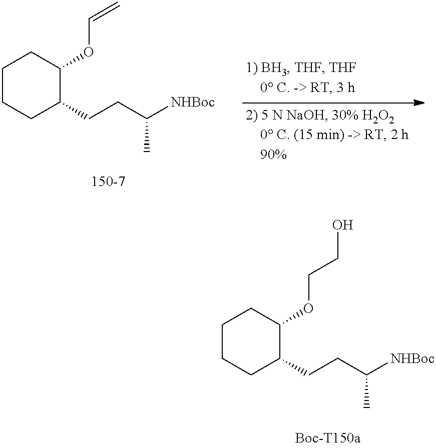

Step T150-1. To a solution of (E)-bromopropene (15 g, 124 mmol) in THF/Et₂O (1:1, 150 mL) was added a 1.7 M solution of t-BuLi in hexanes (146 mL, 248 mmol) at −100° C. under N₂. The reaction was then stirred at −78° C. for 1 h. The reaction was returned to −100° C. and a solution of 104-4 (15 g, 62 mmol) in THF/Et₂O (1:1, 100 mL) added over a period of 30 min. After the addition, the reaction was stirred 1 h at −78° C., then quenched with a saturated solution of NaHCO₃ (aq). The mixture was extracted with Et₂O (3×). The combined organic phase was washed with brine, dried over Na₂SO₄, filtered, and the filtrate concentrated under reduced pressure. The crude product was purified by flash chromatography (5% Et₂O/hexanes) to give a 1.2:1 mixture of diastereoisomers with different configurations at the free hydroxylcarbon atom, 6.95 g for the (R)-isomer, 150-1, and 8.37 g for the (S)-isomer, 150-2 (87% total yield).

Step T150-2. A suspension of KH (30% in mineral oil, 560 mg, 4.2 mmol) in hexanes (1 mL) was added to a solution of 150-1 (6.0 g, 21.1 mmol) in THF (18 mL) at 0° C. The mixture was stirred 10 min at RT, then added via cannula to a solution of trichloroacetonitrile (3.2 mL, 31.6 mmol) in THF (18 mL) at 0° C. The reaction was stirred 1 h at 0° C., then quenched with saturated solution of NaHCO₃ (aq). The mixture was extracted with Et₂O (3×), the combined organic phase was dried over Na₂SO₄, filtered, and the filtrate concentrated under reduced pressure. Purification of the residue by flash chromatography (5% Et₂O/hexanes+1% Et₃N) provided 150-3 (6.42 g, 71%) containing some minor impurities.

Step T150-3. A solution of 150-3 (6.4 g, 15 mmol) in toluene (150 mL) was heated at 140° C. in a sealed tube for 18 h. The reaction was stopped, evaporated under reduced pressure, and the residue purified by flash chromatography (5% Et₂O/hexane) to yield the 150-4 as a colorless oil (4.2 g, 66%).

Step T150-4. 150-4 (4.2 g, 9.8 mmol) was dissolved in a 1% HCl in MeOH solution (100 mL). The reaction was stirred 1 h at RT, then evaporated to dryness in vacuo. The residue was dissolved in EtOH (100 mL) and a 5 N aqueous solution of NaOH (100 mL) was added at 0° C. The mixture was stirred 4 h at RT, then the EtOH evaporated under reduced pressure. To the residual aqueous phase, THF (100 mL) was added followed by (Boc)₂O (5.36 g, 24.6 mmol). The biphasic mixture was stirred overnight at RT, then diluted with water and extracted with Et₂O (3×). The combined organic phase was washed with brine, dried over MgSO₄, filtered, and the filtrate concentrated under reduced pressure. The purification of the residue thus obtained was done by flash chromatography (gradient., 5% EtOAc/hexanes to 30% EtOAc/hexanes) to afford 150-5 as a colorless oil (1.69 g, 64%).

Step T150-5. To a solution of 150-5 (1.30 g, 4.8 mmol) in EtOH (50 mL) was added 5% Rh/alumina (490 mg). Hydrogen was bubbled through the reaction for 5 min, then the reaction stirred overnight under a hydrogen atmosphere. The reaction was filtered through a Celite pad, which was rinsed with Et₂O, and the combined filtrate and rinses evaporated to dryness under reduced pressure to give 150-6 (1.3 g, 100%).

Step T150-6. To a solution of 150-6 (1.3 g, 4.8 mmol) in ethyl vinyl ether (50 mL) was added mercuric acetate (460 mg, 1.44 mmol) and the solution heated at reflux for 24 h. At that time, another 0.3 eq of mercuric acetate was added and the solution heated at reflux for an additional 24 h. The solution was then cooled to RT, quenched with an aqueous saturated solution of Na₂CO₃, and extracted with Et₂O (3×). The combined organic phase was washed with brine, dried over MgSO₄, filtered, and the filtrate concentrated under reduced pressure. The residue was purified by flash chromatography (5% Et₂O/hexanes with 2% Et₃N) to yield 150-7 as a colorless oil (1.38 g, 97%).

Step T150-7. To a solution of 150-7 (1.35 g, 4.5 mmol) in THF (45 mL) was slowly added, over a period of 15 min at 0° C., a 1 M solution of BH₃.THF (6.9 mL, 6.9 mmol). The mixture was stirred 1 h at 0° C., then 2 h at RT. The solution was then cooled to 0° C. and a 5 N solution of NaOH (10 mL) added, followed by a 30% aqueous solution of H₂O₂ (20 mL). The reaction was stirred 15 min at 0° C., then 2 h at RT. The mixture was extracted with Et₂O (3×). The combined organic phase was washed with brine, dried over MgSO₄, filtered, and the filtrate concentrated under reduced pressure. The residue was purified by flash chromatography (20% EtOAc(hexanes) to afford Boc-T150a (1.27 g, 90%)

The other diastereomeric tether, Boc-T150b, was accessed using an identical sequence starting from 150-2.

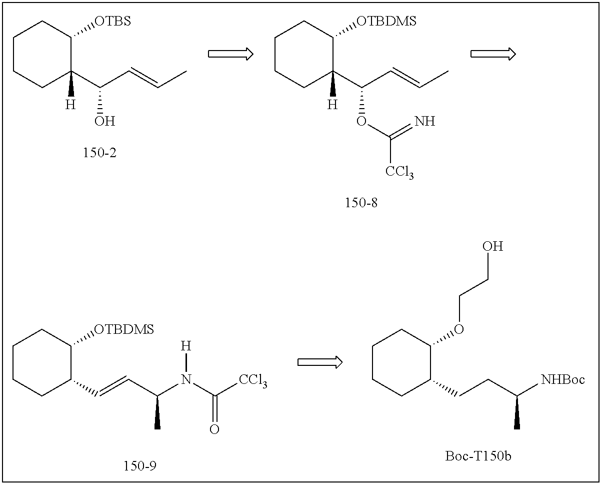

### W. Standard Procedure for the Synthesis of Tether T151

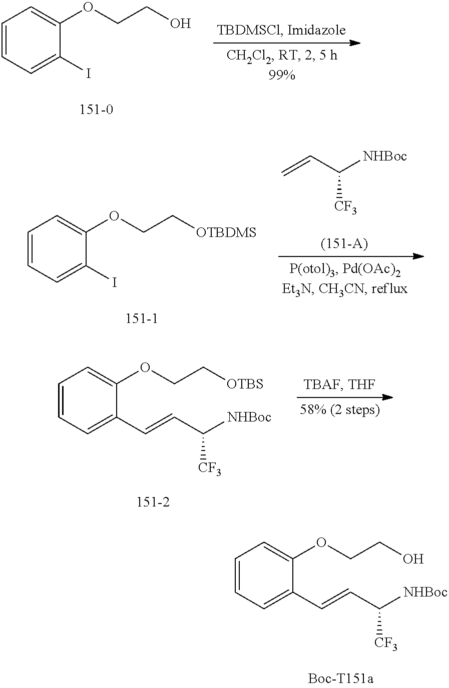

Step T151-1. To the iodophenol derivative 151-0 (5.10 g, 19.3 mmol, 1.0 eq) in dichloromethane (80 mL), was added t-butylchlorodimethylsilane (3.19 g, 21.3 mmol, 1.1 eq) and, last, imidazole (1.45 g, 21.3 mmol, 1.1 eq). The milky solution was stirred at RT for 2.5 h. A saturated aqueous ammonium chloride solution (100 mL) was added and the mixture vigorously stirred for 5 min. The phases were allowed to separate and the aqueous phase extracted with dichloromethane (2×). The organic phases were combined, washed with brine, dried over Na₂SO₄, filtered, and the filtrate concentrated under reduced pressure. The resulting yellow liquid was purified on a short silica gel column (gradient, 4% to 10% EtOAc:Hexanes) to obtain 151-1 as a colorless liquid (7.25 g, 99%).

TLC: R_{f}=0.40 (15% EtOAc:Hexanes; detection: KMnO₄)

Step T151-2. 151-1 (541 mg, 1.43 mmol, 1.0 eq), 151-A (see synthesis following, 403 mg, 1.79 mmol, 1.25 eq), tri(o-tolyl)phosphine (44 mg, 0.143 mmol, 0.1 eq) and palladium diacetate (16 mg, 0.072 mmol, 0.05 eq) were dissolved/suspended in anhydrous acetonitrile (10 mL) under dry nitrogen. Triethylamine (402 μL, 2.864 mmol, 2.0 eq) was then added. The resulting pale yellow mixture was heated at reflux. The mixture quickly darkened and became black after 3 h of heating. After 23 h, heating was stopped, the mixture cooled to RT, and the solvent evaporated to dryness under reduced pressure. The residue was dissolved in 10% EtOAc:Hexanes (8-10 mL) and filtered through a short silica pad with washing with an additional 40 mL of 10% EtOAc:Hexanes. After evaporation of the combined filtrate and washings under reduced pressure, the resulting yellow oil was further purified by flash chromatography (5% EtOAc:Hexanes) to provide 151-2 as a bright yellow oil (627 mg). The ¹H NMR and LC-MS analyses indicated that there was some 151-A in this material, which was used in the next step without further purification.

TLC: R_{f}=0.25 (5% EtOAc:Hexanes; detection: vanillin, CAM, KMnO₄).

Step T151-3. 151-2 (627 mg, 1.32 mmol, 1.0 eq) was dissolved in THF (13.2 mL). A 1 M solution of tetra-N-butylammonium fluoride in THF (1.58 mL, 1.58 mmol, 1.2 eq) was added dropwise over a period of 1 min. The solution immediately turned a deep yellow. The reaction was stirred at RT for 2 h, after which TLC (30% EtOAc:Hexanes) indicated a clean conversion. The mixture was quenched with saturated aqueous NaCl solution (25 mL) and stirred vigorously for 5 min. The phases were allowed to separate and the aqueous phase extracted with ethyl acetate (2×). The organic phases were combined, washed with brine, dried over Na₂SO₄, filtered, and the filtrate concentrated under reduced pressure. The resulting yellow oil was purified by flash chromatography (30% EtOAc:Hexanes). Only the most pure fractions were collected, as a slightly more polar impurity was hard to separate from the desired product. Boc-T151a was isolated as white crystals, 300 mg (58% over two steps).

TLC: R_{f}=0.30 (30% EtOAc:Hexanes; detection: CAM);

HPLC/MS: Gradient A4, t_{R}=7.00 min, [M+Na]⁺ 384;

Chiral HPLC analysis: 88% ee;

¹H NMR (CDCl₃): δ 7.40 (dd, 1H, J₁=7.6, J₂=1.6), 7.25 (td, 1H, J₁=8.8, J₂=1.6), 7.08 (d, 1H, J=16.0), 6.95 (t, 1H, J=7.0), 6.87 (d, 1H, J=8.2), 6.16 (dd, 1H, J=16.0, J₂=6.5), 5.17 (bs, 1H). 4.97 (bs, 1H), 4.11 (t, 2H, J=5.0), 3.99 (t, 2H, J=5.0), 2.48 (bs, 1H), 1.47 (s, 9H).

The enantiomeric tether with the (S)-configuration, Boc-T151b is accessed by the same procedure, but starting from the enantiomeric amino acid, 151-B.

### Y. Standard Procedure for the Synthesis of Reagent 151-A

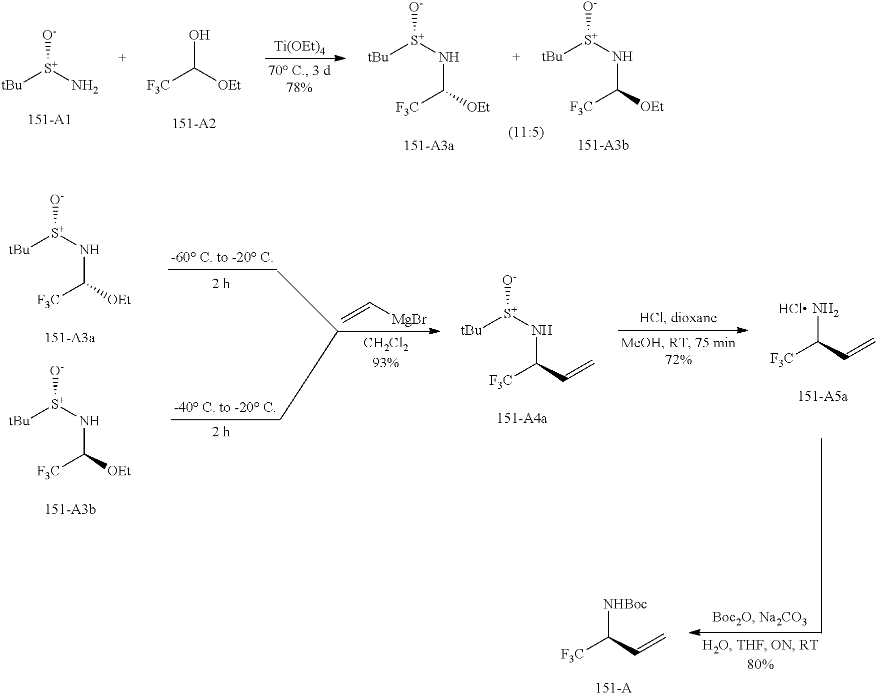

Step T151-A. (S)-(−)-2-Methyl-2-propanesulfinamide 151-A1 (1.84 g, 15.2 mmol, 1.1 eq) was mixed with trifluoroacetaldhyde ethyl hemiacetal (151-A2, 1.99 g, 13.8 mmol, 1.0 eq). Titanium tetraethoxide (4.3 mL, 20.7 mmol, 1.5 eq), was added to form a clear, thick solution which was heated at 70° C. with a reflux condenser under nitrogen for 3 d. By then, the solution had gradually become yellow. The reaction mixture was allowed to cool to RT, diluted with 100 mL of ethyl acetate, then poured into 100 mL of saturated aqueous NaCl solution under vigorous stirring. The biphasic mixture was filtered through Celite and the filter cake rinsed with ethyl acetate. The phases were allowed to separate and the aqueous phase extracted with ethyl acetate (1×). The organic phases were combined, washed with brine, dried over Na₂SO₄, filtered, and the filtrate concentrated under reduced pressure to leave a yellow oil. TLC (50% EtOAc: Hexanes) revealed that the two product diastereomers each had a significantly different R_{f} (0.2 vs. 0.4). Flash chromatography (gradient, 40% to 60% EtOAc:Hexanes) afforded 151-A3a as white powder (1.84 g, 54%) and 151-A3b as white crystals (830 mg, 24%). Both compounds appeared pure by NMR spectroscopy and TLC.
151-A3a, TLC: R_{f}=0.15 (50% EtOAc:Hexanes; detection: vanillin (blue green antispots);
151-A3b, TLC: R_{f}=0.35 (50% EtOAc:Hexanes; detection: vanillin (blue green antispots).

Step T151-B. 151-A3a (830 mg, 3.36 mmol, 1.0 eq) was dissolved in dichloromethane (26 mL) under nitrogen and the solution cooled to −60° C. A 1.0 M solution of vinylmagnesium bromide in THF (8.4 mL, 8.4 mmol, 2.5 eq) was added dropwise over a period of 10 min, after which the reaction was left to stir at −60° C. for an additional 45 min. The temperature was gradually allowed to rise to −20° C. over a period of 75 min. At that time, approximately 50 mL of an aqueous solution saturated in NH₄Cl were added to the mixture and it was stirred vigorously for 15 min while allowing to warm to RT. The phases were separated and the aqueous phase extracted with dichloromethane (3×). The organic phases were combined, washed with brine, dried over Na₂SO₄, filtered, and the filtrate concentrated under reduced pressure. The resulting yellow oil was purified by flash chromatography (50% EtOAc:Hexanes). 151-A4a was obtained as a pale yellow oil, 715 mg (93%). The ratio of diastereomers observed by ¹⁹F NMR was 19:1.

TLC: R_{f}=0.30 (50% EtOAc:Hexanes; detection: KMnO₄).

151-A3b was transformed into 151-A4a using the exact same procedure except for the temperature used for addition of the vinylmagnesium bromide (−40° C. instead of −60° C.).

Step T151-C. 151-A4a (715 mg, 3.119 mmol, 1.0 eq) was dissolved in methanol (1.5 mL). A 4 M solution of hydrogen chloride in 1,4-dioxane (1.5 mL, 6.24 mmol, 2.0 eq) was added dropwise over a period of 1 min. The solution was allowed to stir at RT for 75 minutes, after which TLC indicated a complete reaction. The solvents were evaporated under reduced pressure to yield a sticky oil. About 400 μL of methanol were added to dissolve the oil, then 15-20 mL of cold ether was added with stirring, which precipitated the hydrochloride salt. This solid was filtered under vacuum and rinsed with 5-10 mL cold ether. 151-A5a was obtained as a white powder, 361 mg (72%).

TLC: R_{f}=baseline (50% EtOAc:Hexanes; detection: KMnO₄).

Step T151-D. 151-A5a (361 mg, 2.24 mmol, 1.0 eq) was dissolved in THF (7 mL) and water (7 mL). Sodium carbonate (321 mg, 3.02 mmol, 1.1 eq) and di-t-butyl-dicarbonate (660 mg, 3.02 mmol, 1.1 eq) were successively added to the biphasic mixture. The resulting solution was stirred overnight at RT. Distilled water (˜30 mL) was added to the mixture. The phases were allowed to separate and the aqueous phase extracted with EtOAc (3×). The organic phases were combined, washed with brine, dried over Na₂SO₄, filtered, and the filtrate concentrated under reduced pressure. The resulting yellowish oil was purified by flash chromatography (30% EtOAc:Hexanes) to provide 151-A as white needles, 403 mg (80%).

TLC: R_{F}=0.55 (30% EtOAc:Hexanes; detection: KMnO₄).

¹H NMR (CDCl₃): δ 5.89-5.82 (m, 1H), 5.50-5.40 (m, 2H), 4.83 (br s, 2H), 1.46 (s, 9H).

The enantiomeric amino acid, 151-B, is accessed by the same procedure, but starting from the enantiomeric (R)-(−)-2-methyl-2-propanesulfinamide, 151-B1. This is in turn used to prepare the enantiomeric tether, T151b.

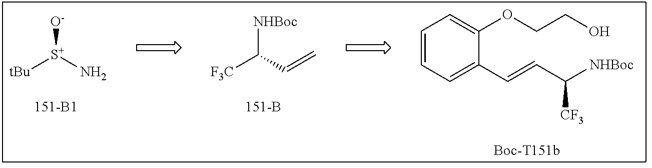

### Z. Standard Procedure for the Synthesis of Tethers T152 and T157

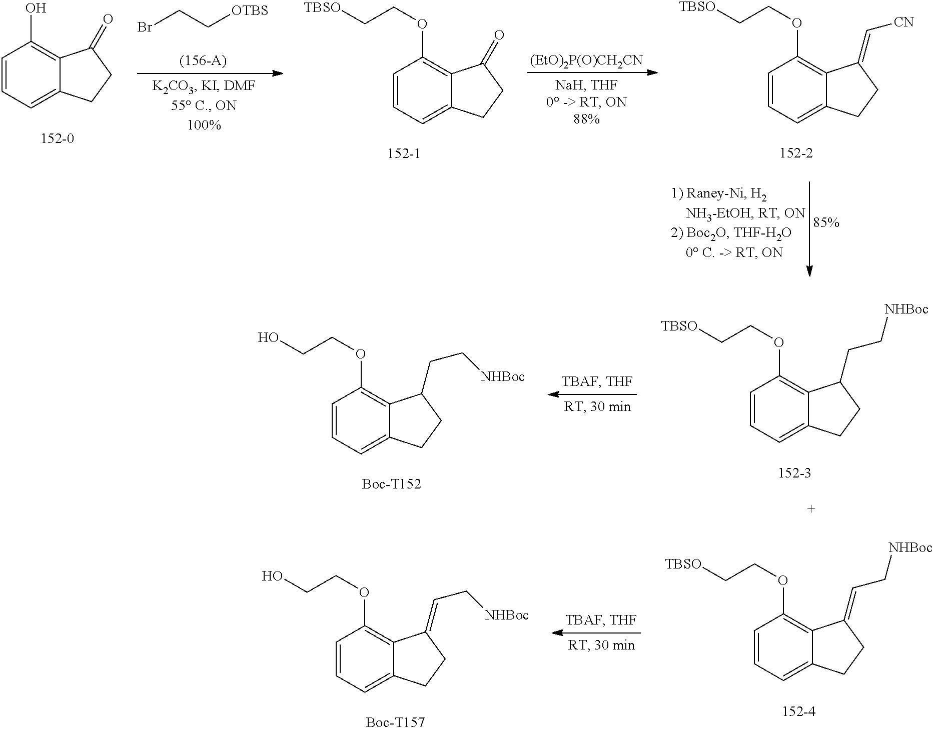

Step T152-1. To a solution of 7-hydroxy-indanone (152-0, 4.15 g, 28 mmol, 1.0 eq, Minuti, L. et. al. Tetrahedron Asymm. 2003, 14, 481-487) in DMF (dry, 85 mL) was added 156-A (synthesis described after that for T156, 10 g, 42 mmol, 1.5 eq), K₂CO₃ (4.84 g, 35 mmol, 1.25 eq) and KI (0.93 g, 5.6 mmol, 0.2 eq). The mixture was stirred at 55° C. (oil bath) overnight (˜16 h) under N₂. The reaction was monitored by TLC (Hexane/EtOAc, 4/1; detection: UV, KMnO₄). The mixture was cooled to rt, H₂O (200 mL) added, the layers separated, then the aqueous layer extracted with EtOAc (3×250 mL). The combined organic phase was washed with brine (100 mL), dried over anhydrous Na₂SO₄, filtered then the filtrate concentrated under reduced pressure and dried under vacuum (oil pump). The residue was purified by flash chromatography (Hexanes/EtOAc, 5/1) to afford 8.6 g (100%) of 152-1 as a colorless oil.

¹H NMR (CDCl₃, 300 MHz): δ 7.47 (m, 1H), 6.99 (d, J=7.6, 1H), 6.84 (d, J=8.2, 1H), 4.19 (t, J=5.8, 2H), 4.04 (t, J=5.6, 2H), 3.06 (t, J=5.6, 2H), 2.64 (m, 2H), 0.89 (s, 9H), 0.10 (s, 6H)

Step T152-2. NaH (1.18 g, 60 wt % in oil, 29.4 mmol, 1.5 eq) was washed with pentane (15 mL), the pentane removed by syringe, and THF (dry, freshly distilled from Na-benzophenone ketyl, 60 mL) added. Diethyl methylcyanophosphonate (3.7 mL, 23.5 mmol, 1.2 eq) was carefully (due to hydrogen gas evolution) added dropwise to the suspension by syringe at 0° C. under N₂. The mixture was stirred at RT for 1.0 h, cooled to 0° C., then a solution of 156-1 (6.0 g, 19.6 mmol, 1.0 eq) in THF (dry, 20 mL) added dropwise. The mixture was allowed to warm to rt, then stirred overnight with TLC monitoring. The solution was concentrated under reduced pressure to give a black residue which was dissolved in H₂O (50 mL) and saturated aq. NaHCO₃ (50 mL). This aqueous solution was extracted with EtOAc (3×150 mL). The combined organic phase was washed with brine (50 mL), dried over anhydrous Na₂SO₄, filtered, and the filtrate concentrated under reduced pressure and dried under vacuum (oil pump) to give a black liquid which was purified by flash chromatography (hexanes/EtOAc, 6/1) to afford 5.7 g (88%) of 152-2 as a white solid. From TLC and NMR analysis, it appeared that a single geometric isomer was isolated.

¹H NMR (CDCl₃, 300 MHz): δ 7.29 (t, J=7.9, 1H), 6.92 (d, J=7.6, 1H), 6.75 (d, J=8.2, 1H), 6.28, 6.27 (s, 1H), 4.15 (t, J=5.0, 2H), 4.00 (t, J=5.2, 2H), 3.08 (s, 2 H), 3.07 (s, 2H), 0.91 (s, 9H), 0.10 (s, 6H).

Step T152-3. To a solution of NH₃ in EtOH (2.0 M, 100 mL) was added 152-2 (5.7 g, 17.3 mmol, 1.0 eq) and Raney 2800 Ni (5.7 g, slurry in H₂O; 100 wt %). The mixture was stirred under H₂ (70 psi) at RT overnight (˜20 h). The mixture was passed through a pad of Celite, then washed with MeOH:Et₃N (5:1, 240 mL). The combined solution was concentrated under reduced pressure and dried under vacuum (oil pump) to give 5.77 g of a yellow oil which was submitted for the subsequent step without further purification. LC-MS indicated that double bond partly remained, ratio could not be easily determined clue to the overlap of signals.

Extension of the hydrogenation time or conduct under higher hydrogen pressure would be expected to give 152-3 almost exclusively.

Step T152-4. The yellow oil was dissolved in THF/H₂O (1/1, 120 mL) and Na₂CO₃ (2.75 g, 26 mmol, 1.5 eq) was added. The mixture was cooled to 0° C. and Boc₂O (4.54 g, 20.8 mmol, 1.2 eq) added in one portion. The reaction was stirred at 0° C. for 30 min, then RT overnight with TLC monitoring of reaction progress. The layers were separated. The aqueous phase was extracted with ether (3×120 mL). The combined organic phase was washed with brine (80 mL), dried over anhydrous Na₂SO₄, filtered, then the filtrate concentrated under reduced pressure and dried under vacuum (oil pump). The resulting residue was purified by flash chromatography (gradient, Hexanes/EtOAc, 20/1 to 15/1) to afford 2.42 g of 152-3, 1.39 g of 152-4 and 2.6 g of mixture of 152-3 and 152-4 as colorless oils [85% overall yield (152-3+152-4) for two steps].

152-3

¹H NMR (CDCl₃, 300 MHz): δ 7.10 (t, J=7.9, 1H), 6.82 (d, J=7.3, 1H), 6.66 (d, J=7.9, 1H), 4.85 (s, br, 1H), 4.00 (m, 4H), 3.50 (m, 5H), 2.21 (m, 1H), 1.87 (m, 2H), 1.65 (m, 1H), 1.44 (s, 9H), 0.91 (s, 9H), 0.09 (s, 6H)

MS: 336 (M⁺+1-Boc)

152-4

MS: 334 (M⁺+1-Boc)

Step T152-5. To a solution of 152-3 (2.42 g, 5.55 mmol, 1.0 eq) in THF (2.0 mL) was added a solution of TBAF (1.0 M in THF, 20 mL, 3.6 eq). The color of the solution changed to green-black immediately. The reaction solution was stirred at RT for 30 min with monitoring by TLC (Hexane/EtOAc, 2/1; detection: UV, CMA). Upon completion, the solution was passed through a pad of silica gel and eluted with EtOAc (100 mL). The combined organic solution was concentrated under reduced pressure and dried under vacuum (oil pump). The residue was purified by flash chromatography on (gradient, hexanes/EtOAc, 5/1 to 3/1 to 2/1) to yield 1.4 g (78%) of Boc-T152 as a colorless sticky oil.

¹H NMR (CDCl₃, 300 MHz): δ 7.11 (t, J=7.9, 1H), 6.84 (d, J=7.6, 1H), 6.66 (d, J=8.2, 1H), 4.98 (s, br, 1H), 4.08 (m, 4H), 3.35 (m, 1H), 3.18 (m, 2H), 3.00 (m, 1 H), 2.80 (m, 1H), 2.23 (m, 1H), 1.99 (m, 1H), 1.78 (m, 2H), 1.45 (s, 9H).

¹³C NMR (CDCl₃, 75 MHz): δ 155.38, 145.90, 134.24, 127.98, 117.36, 108.86, 79.34, 69.38, 61.39, 39.90, 39.57, 33.99, 31.74, 31.48, 28.43

MS: 222 (M⁺+1-Boc)

In a similar manner to that described above, Boc-T157 was obtained from 152-4.
¹H NMR (CDCl₃, 300 MHz): δ 7.13 (t, J=7.9, 1H), 6.88 (d, J=7.3, 1H), 6.70 (d, J=8.2, 1H), 6.47 (s, 1H), 4.66 (s, br, 1H), 4.17 (m, 2H), 4.02 (m, 2H), 3.88 (t, J=6.7, 2H), 2.99 (m, 2H), 2.78 (m, 2H), 2.23 (s, by, 1H), 1.46 (s, 9H)

MS: 264 (M⁺+2H⁺-t-Bu)

### AA. Standard Procedure for the Synthesis of Tether T153

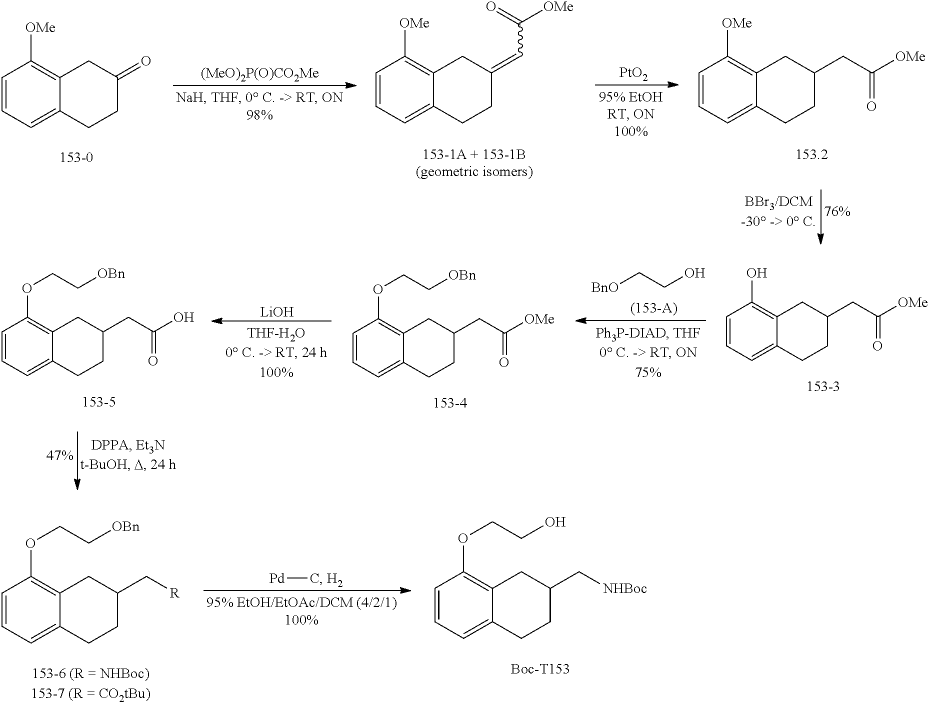

Step T153-1. As described in the literature (Uchikawa, 0. et. al. J. Med. Chem. 2002, 45, 4212-4221; Uchikawa, O. et. al. J. Med. Chem. 2002, 45, 4222-4239), NaH (3.4 g, 60 wt % in oil, 85 mmol, 1.5 eq) was washed with pentane (25 mL), the pentane removed by syringe, and THF (dry, freshly distilled from Na-benzophenone ketyl, 300 mL) then added. To this suspension, trimethylphosphonoacetate (11 mL, 68.1 mmol, 1.20 eq) was carefully (due to hydrogen evolution) added dropwise (˜30 min) by syringe at 0° C. under N₂. The mixture was stirred at RT for 1.0 h, cooled to 0° C., then 8-methoxy-2-tetralone (153-0, 9.0 g, 51 mmol, 1.0 eq) added in one portion. The mixture was allowed to warm to rt, then stirred overnight. Progress of the reaction was monitored by TLC (hexanes/EtOAc, 4/1; detection: UV, KMnO₄). The brown solution was concentrated in vacuo to give a black residue. This residue was dissolved in H₂O (150 mL) and EtOAc (200 mL). The layers were separated and the aqueous phase extracted with EtOAc (3×250 mL). The combined organic phase was washed with brine (150 mL), dried over anhydrous Na₂SO₄, filtered, then the filtrate concentrated under reduced pressure and dried under vacuum (oil pump). The resulting black residue was purified by flash chromatography (hexanes/EtOAc, 5/1) to afford 1.08 g of 153-1A and 10.52 g of 153-1B (total yield 98%) as colorless oils. The structures of 153-1A and 153-1B were deduced from the NMR spectral data.

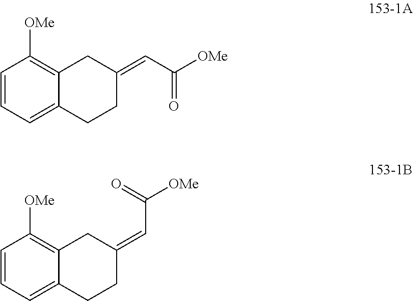

153-1A

¹H NMR (CDCl₃, 300 MHz): δ 7.13 (t, J=7.9, 1H), 6.77 (d, J=7.6, 1H), 6.72 (d, J=8.2, 1H), 5.89 (qu, J=1.5, 1H), 3.83, (s, 3H), 3.71 (s, 3H), 3.52 (s, 2H), 3.12 (m, 2H), 2.86 (t, J=7.0, 2H);

¹³C NMR (CDCl₃, 75 MHz): δ 167.05, 160.13, 156.46, 138.62, 126.53, 123.38, 120.22, 114.07, 107.61, 55.29, 50.85, 33.17, 29.87, 27.52.

153-1B:

¹H NMR (CDCl₃, 300 MHz): δ 7.08 (t, J=7.9, 1H), 6.73 (d, J=6.5, 1H), 6.71 (d, J=7.9, 1H), 3.82 (s, 3H), 3.70 (s, 3H), 3.25 (s, 2H), 2.82 (t, J=7.9, 2H), 2.32 (t, J=7.9, 2H);

¹³C NMR (CDCl₃, 75 MHz): δ 171.80, 154.53, 135.99, 132.74, 127.28, 122.81, 120.04, 119.90, 108.67, 55.44, 51.83, 42.96, 28.24, 26.74.

Step T153-2. To a solution of 153-1B (6.0 g, 25.8 mmol) in 95% EtOH (120 mL) was added PtO₂ (600 mg, 10 wt %). The mixture was stirred under a H₂ filled balloon at RT overnight (˜16 h). The solution was passed through a pad of Celite, eluted with EtOAc, and the resulting organic solution concentrated under reduced pressure and dried under vacuum (oil pump) to afford 6:05 g (100%) of 153-2 as a colorless oil. Similarly, treatment of 153-1A also afforded 153-2, which was verified by ¹H NMR and LC-MS co-injection.

¹H NMR (CDCl₃, 300 MHz): δ 7.08 (t, J=7.9, 1H), 6.71 (d, J=7.3, 1H), 6.65, J=7.9, 1H), 3.81 (s, 3H), 3.71 (s, 3H), 2.94 (m, 1H), 2.82 (m, 2H), 2.41 (m, 2H), 2.20 (m, 2H), 1.93 (m, 1H), 1.46 (m, 1H);

MS: 235 [M+H]⁺.

Step T153-3. 152-2 (7.02 g, 30 mmol, 1.0 eq) was dissolved in DCM (dry, 150 mL). The solution was cooled to −30° C. (dichloroethane-dry ice bath), then a solution of BBr₃ in DCM (1.0 M, 75 mL, 2.5 eq) added dropwise. After addition, the black solution was stirred at −30° C. for 40 min, then 0° C. for 3.0 h, always under N₂, with monitoring by TLC (hexanes/EtOAc, 4/1; detection: UV, KMnO₄). When complete, MeOH (dry, 20 mL) was added dropwise (but not slowly) to the mixture with vigorous stirring and maintaining low temperature, followed by the addition of H₂O (150 mL). The mixture was kept at 0° C. for 2-3 min. The layers were separated, and the aqueous phase extracted with DCM (3×150 mL). The combined organic phase was dried over anhydrous Na₂SO₄, filtered, then the filtrate concentrated under reduced pressure and dried under vacuum (oil pump) to give a black residue which was purified by flash chromatography (Hexanes/EtOAc, 5/1) to afford 5.01 g (76%) of 153-3 as a pale yellow solid.

¹H NMR (CDCl₃, 300 MHz): δ 6.98 (t, J=7.9, 1H), 6.68 (d, J=7.6, 1H), 6.60 (dd, J=7.9, 0.9, 1H), 3.72 (s, 3H), 2.92 (m, 1H), 2.82 (m, 2H), 2.43 (m, 2H), 2.24 (m, 2 H), 1.93 (m, 1H), 1.44 (m, 1H);

¹³C NMR (CDCl₃, 75 MHz): δ 173.50, 153.43, 138.01, 126.15, 122.40, 121.11, 111.86, 51.63, 41.03, 31.09, 29.14, 28.97, 28.72.

Step T153-4. To a solution of 153-3 (5.0 g, 22.7 mmol, 1.0 eq), benzyloxyethanol (153-A, 4.4 mL, 30.6 mmol, 1.35 eq) and triphenylphosphine (8.0 g, 30.6 mmol, 1.35 eq) in THF (dry, 120 mL) was added DIAD (6.0 mL, 30.6 mmol, 1.35 eq) dropwise using a syringe at 0° C. under N₂. The solution was stirred at 0° C. for 30 min, then allowed to warm to RT and stirred overnight. The solution was concentrated under reduced pressure and dried under vacuum (oil pump) to give a pale yellow oil which was purified by flash chromatography (hexanes/EtOAc, 5/1) to obtain 5.98 g (75%) of 153-4 as a colorless oil.

¹H NMR (CDCl₃, 300 MHz): δ 7.31 (m, 5H), 7.06 (t, J=7.9, 1H), 6.71 (d, J=7.6, 1H), 6.64 (d, J=7.9, 1H), 4.65 (s, 2H), 4.14 (m, 2H), 3.85 (m, 2H), 3.68 (s, 3H), 3.00 (m, 1H), 2.82 (m, 2H), 2.40 (m, 2H), 2.24 (m, 2H), 1.93 (m, 1H), 1.42 (m, 1 H).

Step T153-5. To a solution of 153-4 (4.98 g, 14 mmol, 1.0 eq) in THF (35 mL) was added a solution of LiOH.H₂O (2.9 g, 70 mmol, 5.0 eq) in H₂O (35 mL) at 0° C. The mixture was stirred at 0° C. for 30 min, then allowed to warm to room temperature and stirred for 24 h. THF was removed in vacuo, then an aqueous solution of HCl (20 wt %) was added dropwise to adjust the pH to 1.0. The acidified solution was extracted with EtOAc (3×80 mL). The combined organic phase was dried over anhydrous Na₂SO₄, filtered, then the filtrate concentrated under reduced pressure and dried under vacuum (oil pump). The resulting residue was dissolved in toluene (2×25 mL), concentrated again under reduced pressure and dried under vacuum (oil pump) to provide 4.8 g (100%) of 153-5 as a white solid.

¹H NMR (CDCl₃, 300 MHz): δ 7.32 (m, 5H), 7.01 (t, J=7.9, 1H), 6.67 (m, 2H), 4.62 (s, 2H), 4.11 (m, 2H), 3.83 (m, 2H), 3.01 (m, 1H), 2.79 (m, 2H), 2.36 (m, 2H), 2.13 (m, 2H), 1.95 (m, 1H), 1.40 (m, 1H);

¹³C NMR (CDCl₃, 75 MHz): δ 177.57, 158.72, 140.51, 139.55, 130.28, 129.66, 129.54, 127.94, 126.84, 123.20, 110.16, 75.07, 70.75, 69.64, 42.95, 33.42, 31.52, 31.00, 30.84.

Step T153-6. To a solution of 153-5 (4.76 g, 14 mmol, 1.0 eq) in t-BuOH (freshly distilled from Na under nitrogen, 85 mL) was added triethylamine (freshly distlled from CaH₂, 2.2 mL, 15.4 mmol, 1.1 eq) and diphenylphosphoryl azide (DPPA, 3.33 mL, 15.4 mmol, 1.1 eq) under N₂. The solution was refluxed for 24 h under N₂. After returning to rt, the solution was concentrated under reduced pressure and dried under vacuum (oil pump) to give a pale yellow solid. This yellow solid was dissolved in DCM (400 mL), washed successively with a solution of NaOH (1.0 M, 2×80 mL), H₂O (80 mL) and brine (80 mL), dried over anhydrous Na₂SO₄, filtered, then the filtrate concentrated under reduced pressure and dried under vacuum (oil pump) to give a pale yellow solid which was purified by flash chromatography (Hexanes/EtOAc, 5/1) to afford 2.7 g (47%) of 153-6 as a white solid. In addition, 1.39 g of 153-7, the t-butyl ester of 153-5, as a colorless oil, and 1.19 g of 153-8, of undetermined structure, was isolated from the chromatography.

153-6

¹H NMR (CDCl₃, 300 MHz): δ 7.31 (m, 5H), 7.05 (t, J=7.9, 1H), 6.71 (d, J=7.6, 1H), 6.64 (d, J=7.9, 1H), 4.62 (s, 2H), 4.13 (m, 2H), 3.84 (t, J=5.0, 2H), 2.99 (m, 1H), 2.82 (m, 2H), 2.27 (m, 4H), 1.93 (m, 1H), 1.46 (s, 9H), 1.43 (m, 1H);

¹³C NMR (CDCl₃, 75 MHz): δ 172.30, 156.49, 138.20, 137.86, 128.40, 127.65, 125.84, 125.22, 121.28, 107.91, 80.13, 73.35, 68.71, 67.51, 42.66, 31.37, 29.46, 29.13, 28.65, 28.14;

MS: 419 [M+Na]⁺.

153-7

¹H NMR (CDCl₃, 300 MHz): δ 7.33 (m, 5H), 7.05 (t, J=7.9, 1H), 6.71 (d, J=7.6, 1H), 6.64 (d, J=8.2, 1H), 4.65 (s, 2H), 4.15 (dt, J=2.0, 4.7, 2H), 3.85 (t, J=5.0, 2 H), 3.16 (m, 2H), 2.95 (dd, J=16.7, 5.0, 1H), 2.81 (m, 2H), 2.19 (m, 1H), 1.90 (m, 2H), 1.45 (s, 9H), 1.37 (m, 1H);

¹³C NMR (CDCl₃, 75 MHz): δ 156.52, 138.16, 138.06, 128.42, 127.66, 125.89, 124.93, 121.29, 107.99, 73.29, 68.59, 67.49, 46.28, 34.82, 29.06, 28.42, 27.41, 26.60;

MS: 312 [M+H-Boc]⁺.

153-8

¹H NMR (CDCl₃, 300 MHz): δ 7.31 (m, 5H), 7.06 (t, J=7.6, 1H), 6.71 (d, J=7.0, 1 H), 6.65 (d, J=7.9, 1H), 4.65 (s, 2H), 4.15 (m, 2H), 3.85 (t, J=5.3, 2H), 3.27 (m, 2H), 2.94 (m, 1H), 2.81 (m, 2H), 2.22 (m, 1H), 1.90 (m, 2H), 1.30 (m, 2H);

MS: 381 [M+14]⁺.

Step T153-7. To a solution of 153-6 (2.7 g, 6.56 mmol) in 95% EtOH/EtOAc/DCM (4/2/1, 70 mL) was added Pd—C (Degussa, ˜54% H₂O, 675 mg, 25 wt %). The mixture was shaken under H₂ (Parr, 60 psi) at RT for 4.0 h with the reaction monitored by TLC (hexanes/EtOAc, 2/1; detection: UV, CMA). The mixture was passed through a pad of Celite to remove the catalyst and eluted with EtOAc. The combined organic phase was concentrated under reduced pressure and dried under vacuum (oil pump) to give a pale yellow solid which was purified by flash chromatography (gradient, Hexanes/EtOAc, 1/1, then DCM/EtOAc, 1/1) to afford 2.11 g (100%) of Boc-T153 as a white solid.

¹H NMR (CDCl₃, 300 MHz): δ 7.06 (t, J=7.9, 1H), 6.73 (d, J=7.6, 1H), 6.65 (d, J=7.9, 1H), 4.73 (s, 1H), 4.08 (m, 2H), 3.97 (m, 2H), 3.20 (t, J=6.1, 2H), 2.92 (dd, J=16.7, 4.4, 1H), 2.79 (m, 2H), 2.20 (m, 2H), 1.89 (m, 2H), 1.46 (s, 9H), 1.36 (m, 1H);

¹³C NMR (CDCl₃, 75 MHz): δ 156.23, 138.18, 125.98, 124.84, 121.53, 108.03, 79.18, 69.16, 61.59, 46.21, 34.92, 29.03, 28.40, 27.31, 26.56;

MS: 222 [M+H-Boc]⁺.

### BB. Standard Procedure for the Synthesis of Tether T154

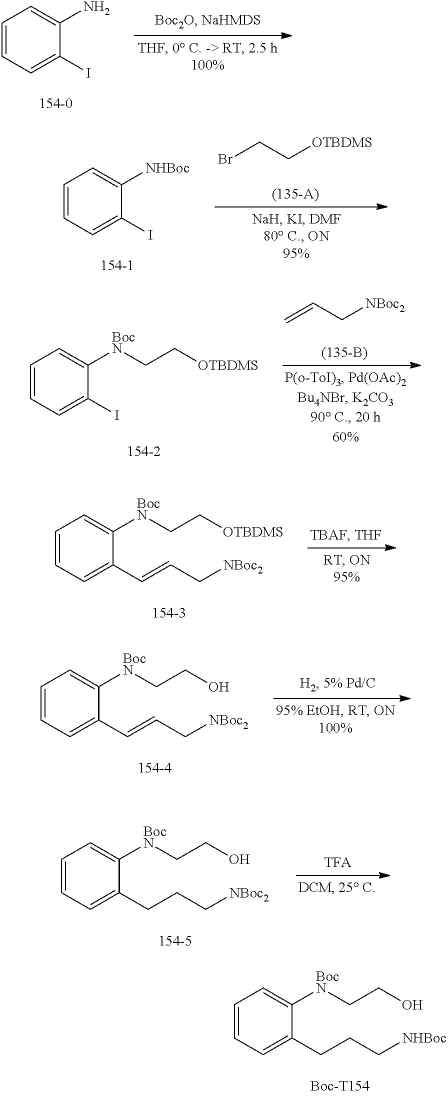

Step T154-1. To a solution of 2-iodoaniline (154-0, 13.1 g, 60.0 mmol, 1.0 eq) in THF (70 mL) at 0° C. was added a solution of NaHMDS (1 M in THF, 132 mL, 132 mmol, 2.2 eq) and the resulting mixture stirred at RT for 25 min. Boc₂O (14.5 g, 66.0 mmol, 1.1 eq) was added and the mixture stirred at RT for 2.5 h. 0.5 M HCl was added and the aqueous phase extracted with EtOAc. The combined organic phase was dried over MgSO₄, filtered, and the filtrate concentrated to dryness under reduced pressure. The resulting residue was purified by flash chromatography (7% EtOAc, 93% hexanes) to give 154-1 (19.0 g, 100%).

Step T154-2. To a solution of 154-1 (12.6 g, 39.6 mmol, 1.0 eq) in DMF (150 mL) were added NaH (60% in oil, 2.1 g, 53.5 mmol, 1.35 eq), KI (32.9 g, 198 mmol, 5.0 eq) and 135-A (12.8 g, 53.5 mmol, 1.35 eq), and the resulting mixture stirred at 80° C. overnight. The mixture was allowed to cool to RT and water added. The aqueous phase was extracted with MTBE and the combined organic phase was extracted with brine. The organic phase was dried over MgSO₄, filtered, and the filtrate concentrated to dryness under reduced pressure to give 154-2 as a white solid (18 g, 95%).

Step T154-3. To a solution of 154-2 (17.3 g, 36.0 mmol, 1.0 eq) in DMF (100 mL) were added 135-B (13.9 g, 54.0 mmol, 1.5 eq), P(o-Tol)₃ (1.1 g, 3.6 mmol, 0.1 eq), K₂CO₃ (9.9 g, 72.0 mmol, 2.0 eq) and Bu₄NBr (1.16 g, 3.6 mmol, 0.1 eq), and the resulting mixture degassed with Ar. Pd(OAc)₂ (0.8 g, 3.6 mmol, 0.1 eq) was added and the mixture again degassed with Ar. The resulting mixture was stirred at 90° C. for 20 h. Water was added and the aqueous phase extracted with ether. The combined organic phase was extracted with brine, dried over MgSO₄, filtered, and the filtrate concentrated to dryness under reduced pressure. The residue was purified by flash chromatography (11% EtOAc, 89% hexanes) to give the compound 154-3 (13.0 g, 60%) plus some recovered starting material (7.8 g).

Step T154-4. To a solution of 154-3 (11.9 g, 19.6 mmol, 1.0 eq) in THF (60 mL) was added a solution of TBAF (1 M in THF, 39.2 mL, 39.2 mmol, 2.0 eq) and the resulting mixture stirred at RT overnight. Water was added and the aqueous phase extracted with EtOAc. The combined organic phase was extracted with brine, dried over MgSO₄, filtered, and the filtrate concentrated to dryness under reduced pressure. The residue was purified by flash chromatography (40% EtOAc, 60% hexanes) to give 154-4 as a solid (9.2 g, 95%).

HPLC/MS: Gradient A4, t_{R}=9.81 min, [M]⁺ 492, [M+Na]⁺ 515

Step T154-5. To a solution of 154-4 (3.3 g, 6.7 mmol, 1.0 eq) in 95% EtOH (20 mL) was added 5% Pd/C (300 mg). Hydrogen was bubbled through the mixture, which was then stirred under a hydrogen atmosphere overnight. Nitrogen was bubbled through the mixture to remove excess hydrogen, then the mixture filtered through a Celite pad and the filter rinsed with EtOAc. The combined filtrate was concentrated under reduced pressure to give 154-5 in quantitative yield.

HPLC/MS: Gradient A4, t_{R}=10.41 min, [M]⁺ 494, [M+Na]⁺ 517

Step T154-6. To synthesize Boc-T154, one of the Boc groups is selectively removed from 154-5 using the procedure as described for T135 (Step 135-4), T136 (Step 136-4) and T137 (137-6) by treatment of 154-5 with TFA in DCM at RT with monitoring by TLC to ensure no loss of the other Boc groups.

### CC. Standard Procedure for the Synthesis of Tether T156

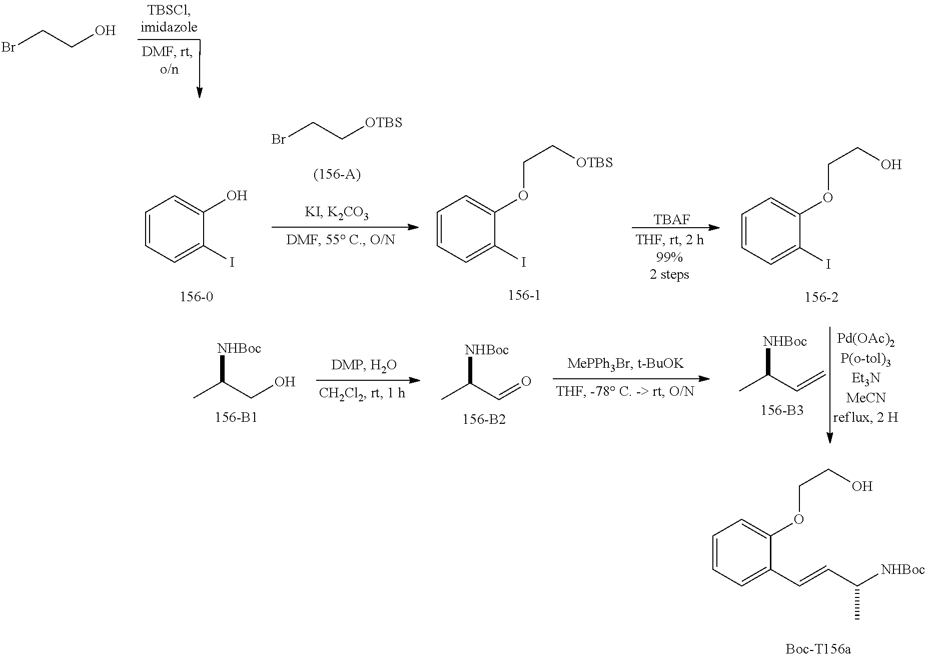

Step T156-1. To a solution of 2-bromoethanol (50 g, 400 mmol, 1 eq) and imidazole (54.5 g, 800 mmol, 2 eq) in THF (1600 mL) was added TBDMSCl (63.3 g, 420 mmol, 1.05 eq) and the solution reaction became milky. After overnight agitation, Et₂O was added (1600 mL) and the mixture washed with a saturated aqueous solution of NH₄Cl (2×250 mL) and brine (250 mL). The organic phase was dried with MgSO₄, filtered, and the filtrate evaporated under reduced pressure to give 156-A (97 g, 405 mmol, >100%) as an oil. When imidazole was seen remaining in this material, it can be removed by dissolution in Et₂O, washing with 1 M citrate buffer, then evaporation of the organic under reduced pressure. Alternatively, 156-A was available commercially (Aldrich cat. no. 428426).

Step T156-2. A solution of 2-iodophenol (156-0, 7.66 g, 34.8 mmol, 1.0 eq) in DMF (115 mL) was degassed under high vacuum for 10 min. Nitrogen was introduced into the flask and 156-A (10 g, 41.8 mmol, 1.2 eq), KI (1.16 g, 6.96 mmol, 0.2 eq) and K₂CO₃ (6.01 g, 43.5 mmol, 1.25 eq) were added. The mixture was stirred at 55° C. overnight under nitrogen. Solvent was removed under vacuum (oil pump), water (150 mL) added and the aqueous phase extracted with Et₂O (3×150 mL). The combined organic phase was washed with 1 M Na₂CO₃ (50 mL) and brine (200 mL), dried with MgSO₄, filtered, and the filtrate concentrated under reduced pressure to give 156-1 which was sufficiently pure to be used directly for the next step.

Step T156-3. To a solution of 156-1 (from previous reaction) in THF (350 mL) was added TBAF (1 M solution in THF, 63 mL, 63 mmol, 1.5 eq). The reaction was stirred for 2 h. Et₂O (600 mL) was added and the organic phase washed with a saturated solution of aq. NH₄Cl (2×100 mL) and brine (100 mL), dried with MgSO₄, filtered, and the filtrate concentrated under reduced pressure. The residue was purified by flash chromatography (40% EtOAc/hexanes) to afford 9.1 g (99%, 2 steps) of 156-2.

Step T156-4. A solution of 156-2 (4.55 g, 17.2 mmol, 1.0 eq) and 156-B3 (3.24 g, 18.9 mmol, 1.1 eq) in MeCN (110 mL) was degassed with argon for 45 min. To the degassed solution was added Et₃N (4.8 mL, 34.4 mmol, 2.0 eq), P(o-tol)₃ (524 mg, 1.72 mmol, 0.1 eq) and Pd(OAc)₂ (193 mg, 0.86 mmol, 0.05 eq). The reaction was heated to reflux with agitation for 2 h under argon. After cooling to rt, the solvent was removed in vacuo and the residue dissolved in CH₂Cl₂ (100° mL) and water (100 mL). The phases were separated and the aqueous phase extracted with CH₂Cl₂ (2×100 mL). The organic phase was dried with MgSO₄, filtered, and the filtrate evaporated under reduced pressure. The residue was purified using flash chromatography (30% EtOAc/hexanes) to give Boc-T156a (2.98 g, 9.7 mmol, 56%) as a brown solid. Note that without N-protection, this compound exhibits some instability.

HPLC/MS: Gradient A4, t_{R}=6.77 min, [M+Na]⁺ 330

The enantiomeric tether, Boc-T156b, is accessed by the same procedure, but starting from the enantiomeric amino alcohol (R)-(−)-2-amino-1-propanol, 156-C1.

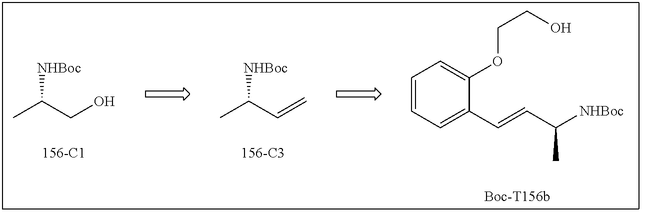

### DD. Standard Procedure for the Synthesis of Reagent 156-B3

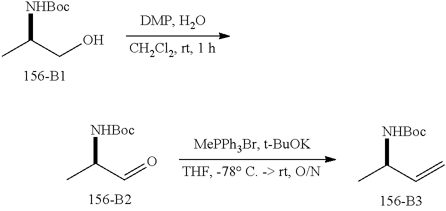

Step T156-5. To a solution of 156-B1 (7.01 g, 40 mmol, 1.0 eq) in CH₂Cl₂ (180 mL) was added DMP (23.8 g, 56 mmol, 1.4 eq). CH₂Cl₂ (containing 0.1% H₂O, 820 mL, 45 mmol, 1.125 eq) was then added over 30 min. The solvent was evaporated to dryness in vacuo and the residue dissolved in ether (500 mL) and a mixture of an saturated aqueous solution of NaHCO₃ and a solution of 10% Na₂S₂O₃ (1:1) (400 mL). This mixture was agitated for 1 h, the phases separated, and the organic phase washed with water (100 mL) and brine (500 mL). The organic phase was dried with MgSO₄, filtered, and the filtrate evaporated under reduced pressure to provide 156-B2 (6.2 g) that was used directly for next step.

Step T156-6. To a solution of MePPh₃Br (31.4 g, 88 mmol, 2.2 eq) in THF (250 mL) was added t-BuOK (8.98 g, 80 mmol, 2.0 eq). The solution was agitated 90 min, cooled to −78° C. and 156-B2 in THF (150 mL) added by cannula. The ice bath was removed and the reaction agitated at RT overnight. A saturated aq. solution of NH₄Cl (100 mL) was added to dissolve the precipitated salts, the mixture agitated 5 min, and the phases separated. The aqueous phase was extracted with ether (2×200 mL). The combined organic phase was washed with brine (50 mL), dried with MgSO₄, filtered, and the filtrate evaporated under reduced pressure to obtain a residue that was purified by flash chromatography (10% EtOAc/hexanes) to yield 156-B3 (70%, 2 steps) as a white solid. The enantiomeric aminoalkene, Boc-156C-3, is accessed by the same procedure, but starting from the enantiomeric amino alcohol (R)-(−)-2-amino-1-propanol, 156-C1.

### EE. Standard Procedure for the Synthesis of Tether T158

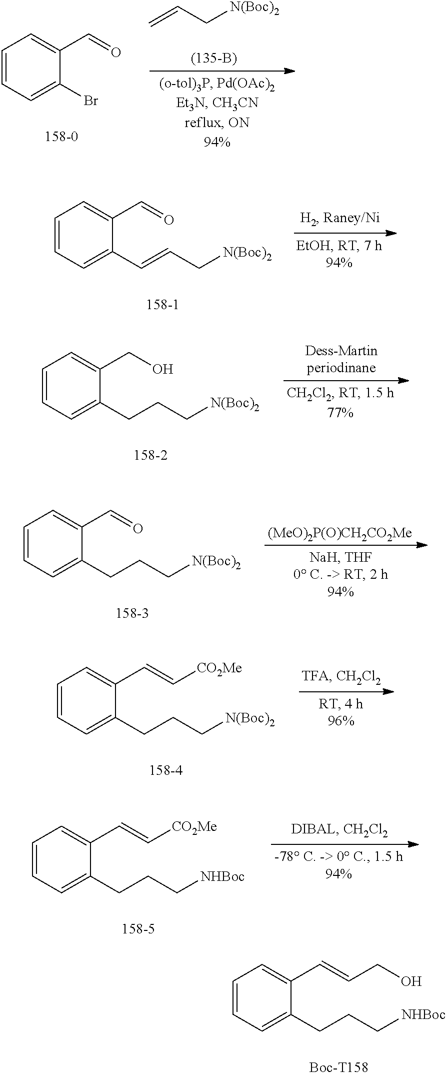

Step T158-1. To a solution of 2-bromobenzaldehyde (158-0, 9.6 g, 51.9 mmol, 1.0 eq) in CH₃CN (300 mL) were added 135-B (14.7 g, 57.1 mmol, 1.1 eq), (o-tol)₃P (1.6 g, 5.2 mmol, 0.1 eq), Pd(OAc)₂ (584 mg, 2.6 mmol, 0.05 eq) and Et₃N (14.6 mL, 103.8 mmol, 2.0 eq). The resulting mixture was stirred at reflux overnight. The mixture was cooled to RT and the solvent evaporated under reduced pressure. Water was added and the aqueous phase extracted with CH₂Cl₂. The organic phase was extracted with brine (2×). The combined organic phase was dried over MgSO₄, filtered, and the filtrate concentrated to dryness under reduced pressure. The residue was purified by flash chromatography (15% EtOAc, 85% hexanes) to afford the 158-1 as a yellow oily semi-solid (17.5 g, 94%).

TLC: R_{f}=0.49 (30% EtOAc, 70% hexanes; detection: UV, Mo/Ce).

Step T158-2. To a solution of 158-1 (9.3 g, 25.8 mmol, 1.0 eq.) in EtOH (200 mL) was added a suspension of Raney/Ni in water (3 mL) and hydrogen was bubbled into the heterogeneous mixture. The reaction was stirred under a hydrogen atmosphere for 7 h. Nitrogen was then bubbled through the reaction solution to remove excess hydrogen and the mixture filtered through a silica gel pad. The silica was rinsed with 50% EtOAc/Hex and the combined filtrate and washings evaporated under reduced pressure. 158-2 was obtained as a yellow oil (8.8 g, 94%).

TLC: R_{f}=0.29 (30% EtOAc, 70% hexanes; detection: UV, Mo/Ce).

Step T158-3. To a solution of 158-2 (8.8 g, 24.1 mmol, 1.0 eq.) in CH₂Cl₂ (200 mL) was added Dess-Martin periodinane (14.3 g, 33.7 mmol, 1.4 eq). The resulting mixture was stirred at RT for 1.5 h. Aqueous saturated NaHCO₃ solution was added and the aqueous phase extracted with CH₂Cl₂. The combined organic phase was dried over MgSO₄, filtered, and the filtrate concentrated under reduced pressure. The resulting residue was purified by flash chromatography (20% EtOAc, 80% hexanes) to provide 158-3 as a white solid (6.8 g, 77%).

TLC: R_{f}=0.43 (30% EtOAc, 70% hexanes; detection: UV, Mo/Ce).

Step T158-4. To a suspension of NaH (60% in oil, 1.12 g, 28.1 mmol, 1.5 eq) at 0° C. in THF (150 mL) was slowly added the phosphonate (4.1 mL, 28.1 mmol, 1.5 eq). Caution, hydrogen was generated from this reaction. The mixture was stirred 15 min, then 158-3 (6.8 g, 18.7 mmol, 1.0 eq) in THF (50 mL) added. The resulting mixture was stirred at RT for 2 h. Aqueous saturated NH₄Cl solution was added and the aqueous phase extracted with EtOAc. The combined organic phase was dried over MgSO₄, filtered, and the filtrate concentrated under reduced pressure. The residue was purified by flash chromatography (20% EtOAc, 80% hexanes) to yield 158-4 as a pale yellow oil (7.3 g, 94%).

TLC: R_{f}=0.42 (20% EtOAc, 80% hexanes; detection: UV, Mo/Ce).

Step T158-5. To a solution of 158-4 (7.3 g, 17.4 mmol, 1.0 eq.) in CH₂Cl₂ (200 mL) was added TFA (1.9 mL, 26.1 mmol, 1.5 eq). The resulting mixture was stirred at RT for 4 h. Aqueous saturated NaHCO₃ solution was added and the aqueous phase extracted with CH₂Cl₂. The combined organic phase was dried over MgSO₄, filtered, and the filtrate concentrated under reduced pressure. The residue was purified by flash chromatography (30% EtOAc, 70% hexanes) to give 158-5 as a pale yellow oil (5.4 g, 96%).

TLC: R_{f}=0.40 (30% EtOAc, 70% hexanes; detection: UV, Mo/Ce).

Step T158-6. To a solution of a solution of 158-5 (5.4 g, 16.9 mmol, 1.0 eq) at −78° C. in CH₂Cl₂ (100 mL) was added DIBAL (1 M in CH₂Cl₂, 42.3 mL, 42.3 mmol, 2.5 eq). The resulting mixture was stirred at −78° C. for 30 min, then at 0° C. for 1 h. If the reaction was not complete as indicated by TLC, 1 eq additional of DIBAL was added. A 1 M solution of Rochelle salts was added and the mixture stirred 1 h. The aqueous phase was extracted with CH₂Cl₂ until TLC indicated no additional material was being extracted. The combined organic phase was dried over MgSO₄, filtered, and the filtrate concentrated under reduced pressure. The residue was purified by flash chromatography (60% EtOAc, 40% hexanes) to provide Boc-T158 as a colorless oil (4.6 g, 94%).

TLC: R_{f}=0.17 (50% EtOAc, 50% hexanes; detection: UV, Mo/Ce); HPLC/MS: Gradient A4, t_{R}=6.83 min, [M]⁺ 291, [M+Na]⁺ 314.

### FF. Standard Procedure for the Synthesis of Tether T159

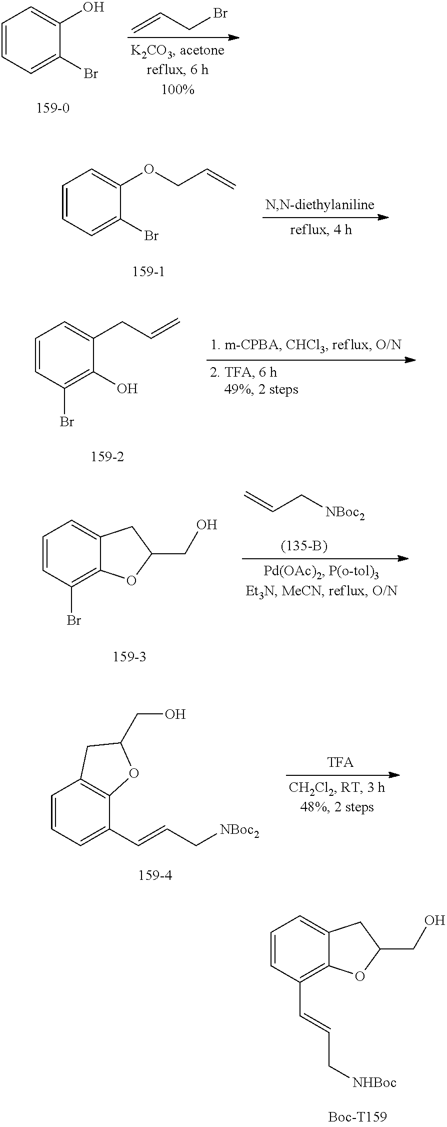

Step T159-1. To a solution of 2-bromophenol (159-0, 45 g, 260 mmol, 1.0 eq) in acetone (1.3 L) was added anhydrous potassium carbonate (71.9 g, 520 mmol, 2.0 eq) and allyl bromide (34.6 g, 24.2 mL, 286 mmol, 1.1 eq). The suspension was stirred at reflux under argon for 6 h. The reaction was cooled to RT, then the solvent removed under vacuum, cold water (500 mL) added and the aqueous phase extracted with ether (3×500 mL). The combined organic phase was washed with water (200 mL) and brine (100 mL), dried with magnesium sulfate, filtered, and the filtrate concentrated under vacuum to give 159-1 as an oil (55.6 g, 213 mmol, 100%) that was used in the next step without further purification.

TLC: R_{f}=0.32 (25% CH₂Cl₂/hexanes).

Step T159-2. A solution of 159-1 (51.0 g, 239 mmol, 1.0 equiv) in N,N-diethylaniline (36 mL, 1:1 v/v) was stirred at reflux for 4 h. The reaction could be followed by ¹H NMR. The solution was allowed to cool to RT and dilute HCl added (300 mL). The aqueous phase was extracted with ether (3×300 mL). The combined organic phase was dried with magnesium sulfate, filtered, and the filtrate concentrated under vacuum. The residue was dissolved in ether (500 mL) and extracted with 1 N NaOH (4×250 mL). The aqueous phase was acidified to pH 2-3 with 6 N HCl, then extracted with ether (3×250 mL). The combined organic phase was dried with magnesium sulfate, filtered, and the filtrate concentrated under vacuum to provide 159-2 as an oil (46 g), contaminated with some diethylaniline, that was used as obtained in the next step.

Step T159-3. To a solution of 159-2 (46 g) in CHCl₃ (2.4 L) was added m-CPBA (80.5 g, 359 mmol, 1.5 eq) and TFA (1.8 mL, 24 mmol, 0.1 eq). The reaction was stirred at reflux overnight. TFA (1.8 mL) was added and reaction stirred for 3 h. Another portion of TFA (14.4 mL) was added and reaction stirred an additional 3 h. The reaction was cooled to RT, then washed with a saturated solution of sodium bicarbonate (2×500 mL) and brine (500 mL). The organic phase was dried with magnesium sulfate, filtered, and the filtrate concentrated under vacuum to give an orange solid that was purified by flash chromatography (gradient, 20%-30%-40% EtOAc/hexanes). Two product-containing fractions were obtained. The first (20 g) was repurified by flash chromatography with the same conditions as above to afford 12.0 g (52.4 mmol, 21.9%, 2 steps) of 159-3. The second (14.9 g, 65.0 mmol, 27.2%, 2 steps) contained pure 159-3.

Step T159-4. To a solution of 159-3 (2.67 g, 11.6 mmol, 1.0 eq), 135-B (3.29 g, 12.8 mmol, 1.1 eq) and Et₃N (3.2 mL, 23.2 mmol, 2.0 eq) in MeCN (preferably degassed, 72.5 mL) was added P(o-tol)₃ (706 mg, 2.32 mmol, 0.2 eq) and Pd(OAc)₂ (260 mg, 1.16 mmol, 0.1 eq). The mixture was stirred at reflux overnight under argon. The solution was concentrated under vacuum, water (250 mL) and CH₂Cl₂ (250 mL) added and the phases separated. The aqueous phase was extracted with CH₂Cl₂ (2×250 mL). The combined organic phase was dried with magnesium sulfate, filtered, and the filtrate concentrated under vacuum to give an oil which was purified by flash chromatography (30% EtOAc/hexanes) to afford 5 g (>100%) of a 2:1 mixture of the product (159-4) and starting material (159-3).

Step T159-5. To a solution of 159-4 (5 g, 12.3 mmol) in CH₂Cl₂ (60 mL) was added TFA (1.1 mL, 15 mmol, 1.22 eq) The mixture was stirred at RT for 3 h, Ether (250 mL) was then added and the organic phase washed with a saturated solution of sodium bicarbonate (50 mL) and brine (50 mL). The organic phase was dried with magnesium sulfate, filtered, and the filtrate concentrated under vacuum to give a yellow residue which was purified by flash chromatography (gradient, 30%-40%-50% EtOAc/hexanes) to afford 1.69 g (48%, 2 steps) of Boc-T159 as a yellow oil.

TLC: R_{f}=0.35 (50% EtOAc/hexanes)

### GG. Standard Procedure for the Synthesis of Tether T160

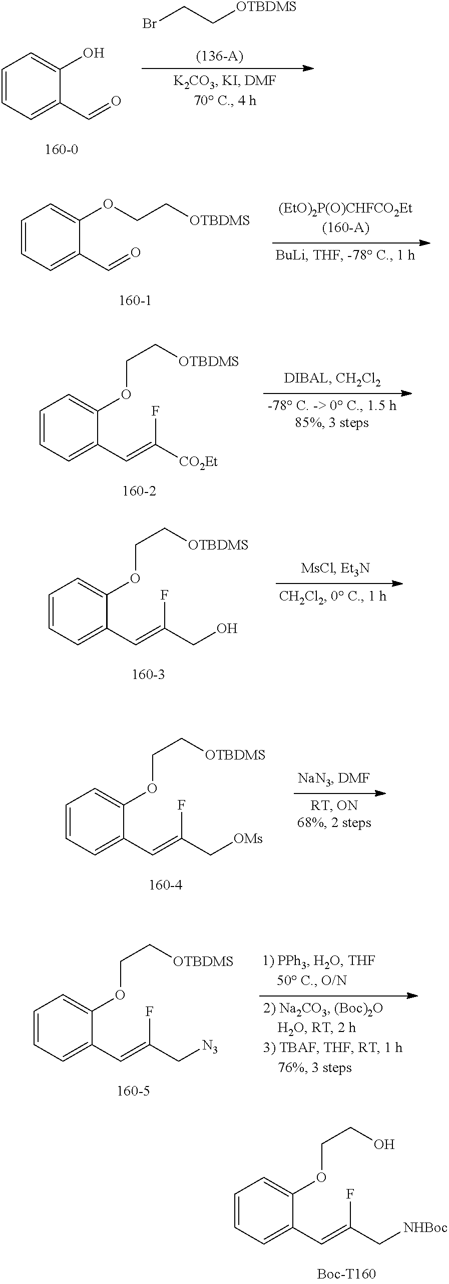

Step T160-1. To a solution of 2-hydroxybenzaldehyde (160-0, 1.2 g, 4.8 mmol, 1.0 eq) in DMF (20 mL) were added potassium carbonate (1.5 g, 10.8 mmol, 1.1 eq), potassium iodide (332 mg, 2.0 mmol, 0.2 eq) and 136-A (4.2 mL, 19.6 mmol, 2.0 eq). The resulting mixture was stirred at 70° C. for 4 h. The solution was cooled to RT and brine added. The aqueous phase was extracted with ether and the combined organic phase was extracted with brine (2×). The organic phase was dried over MgSO₄, filtered, and the filtrate concentrated under reduced pressure. The residue was purified by flash chromatography (15% EtOAc, 85% hexanes) to give 160-1 (3.0 g, >100%, contains trace of 136-A as detected by ¹H NMR).

TLC: R_{f}=0.55 (20% EtOAc, 80% hexanes; detection: UV, Mo/Ce).

Step T160-2. To a solution of phosphonate 160-A (3.6 g, 15.0 mmol, 1.5 eq, Alagappan Thenappan and Donald J. Burton J. Org. Chem. 1990, 4639) at −78° C. in THF (150 mL) was added a solution of n-BuLi (2 M in pentane, 7.5 mL, 15.0 mmol, 1.5 eq). The resulting mixture was maintained at −78° C. for 10 min, then 160-1 (2.8 g, 10.0 mmol, 1.0 eq) in THF (50 mL) added and the resulting mixture stirred at −78° C. for 45 min. Saturated aqueous NH₄Cl was added and the aqueous phase extracted with EtOAc. The combined organic phase was dried over MgSO₄, filtered, and the filtrate concentrated under reduced pressure. The residue was purified by flash chromatography (10% EtOAc, 90% hexanes) to provide 160-2 (3.9 g, 105%, contains a trace of 136-A as detected by ¹H NMR).

TLC: R_{f}=0.58 (20% EtOAc, 80% hexanes; detection: UV, Mo/Ce).

Step T160-3. To a solution of ester 160-2 (3.7 g, 10.0 mmol, 1.0 eq) at −78° C. in CH₂Cl₂ (100 mL) was added a solution of DIBAL (1 M in CH₂Cl₂, 25.0 mL, 25.0 mmol, 2.5 eq, amount critical as loss of TBDMS protection was observed with greater excess of DIBAL). The resulting mixture was stirred at −78° C. for 30 min, then at 0° C. for 1 h. Acetone and Na₂SO₄.10 H₂O were added and the resulting mixture stirred at RT for 2 h. The precipitate was filtered and rinsed with EtOAc and CH₂Cl₂. The solvents were evaporated under reduced pressure and the residue purified by flash chromatography (30% EtOAc, 70% hexanes) to yield 160-3 (2.8 g, 85%, 3 steps).

TLC: R_{f}=0.46 (30% EtOAc, 70% hexanes; detection: UV, Mo/Ce).

Step T160-4. To a solution of 160-3 (2.6 g, 8.0 mmol, 1.0 eq) at 0° C. in CH₂Cl₂ (50 mL) were added Et₃N (5.6 mL, 40.0 mmol, 5.0 eq) and MSCl (1.2 mL, 16.0 mmol, 2.0 eq). The resulting mixture was stirred at 0° C. for 1 h. Water was added and the aqueous phase extracted with CH₂Cl₂. The combined organic phase was dried over MgSO₄, filtered, and the filtrate concentrated under reduced pressure to give the crude mesylate 160-4 (contains trace of MsCl) that was used as obtained this for the next step. TLC: R_{f}=0.24 (20% EtOAc, 80% hexanes; detection: UV, Mo/Ce).

Step T160-5. To a solution of 160-4 (3.2 g, 8.0 mmol, 1.0 eq) in DMF (30 mL) was added NaN₃ (2.6 g, 40.0 mmol, 5.0 eq). The resulting mixture was stirred at RT for 2 h. Water was added and the aqueous phase extracted with ether. The combined organic phase was extracted with brine and the organic phase dried over MgSO₄, filtered, and the filtrate concentrated under reduced pressure to give the crude azide 160-5 (1.9 g, 68%, 2 steps) that was sufficiently pure to be used as obtained for the next step.

TLC: R_{f}=0.68 (20% EtOAc, 80% hexanes; detection: UV, Mo/Ce).

Step T160-6. To a solution of 160-5 (1.9 g, 5.4 mmol, 1.0 eq) in THF (50 mL) were added PPh₃ (2.1 g, 8.1 mmol, 1.5 eq) and water (5 mL). The resulting mixture was heated at 50° C. overnight. (TLC: R_{f}=baseline (20% EtOAc, 80% hexanes; detection: UV, Mo/Ce). The solution was cooled to RT, then water (50 mL), Na₂CO₃ (572 mg, 5.4 mmol, 1.0 eq) and (Boc)₂O (1.2 g, 5.4 mmol, 1.0 eq) added. The resulting mixture was stirred at RT for 2 h. (TLC: R_{f}=0.36 (20% EtOAc, 80% hexanes; detection: UV, Mo/Ce). Water was added and the aqueous phase extracted with EtOAc. The combined organic phase was dried over MgSO₄, filtered, and the filtrate concentrated to dryness under reduced pressure. To the residue in THF (30 mL) was added a solution of TBAF (1 Min THF, 8.1 mL, 8.1 mmol, 1.5 eq). The resulting mixture was stirred at RT for 1 h. Water was added and the aqueous phase extracted with EtOAc. The combined organic phase was dried over MgSO₄, filtered, and the filtrate concentrated under reduced pressure. The residue was purified, by flash chromatography (60% EtOAc, 40% hexanes) to give Boc-T160 (1.3 g, 76%, 3 steps).

TLC: R_{f}=0.10 (20% EtOAc, 80% hexanes; detection: UV, Mo/Ce);

HPLC/MS: Gradient A4, t_{R}=6.51 min, [M]⁺ 311, [M+Na]⁺ 334.

### HH. Standard Procedure for the Synthesis of Tethers T161 and T177

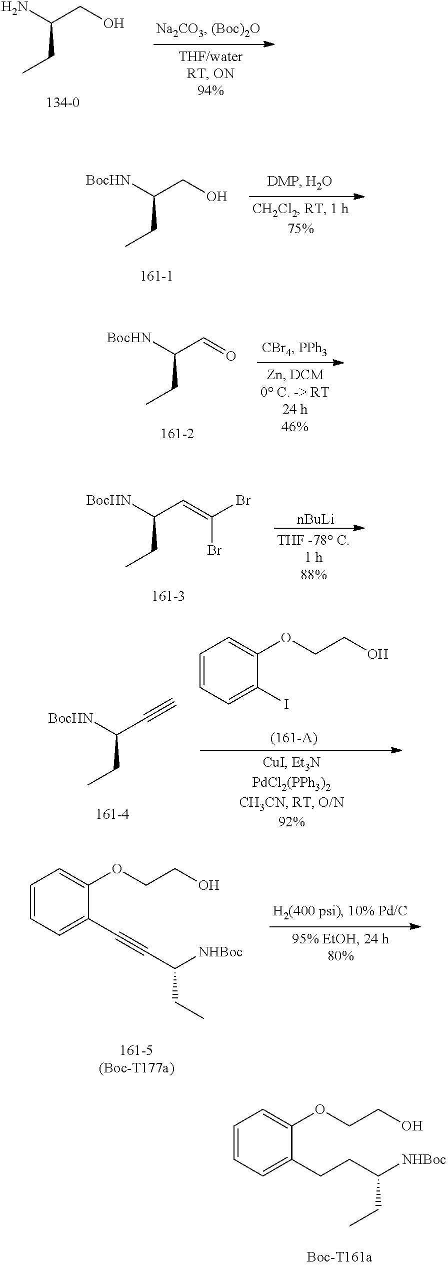

Step T161-1. To a solution of 134-0 [(R)-(−)-2-amino-1-butanol, 5 g, 56 mmol, 1.0 eq] in THF/water (1/1) were added (Boc)₂O (12.9 g, 59 mmol, 1.05 eq) and sodium carbonate (7.12 g, 67 mmol, 1.2 eq). The solution was stirred at RT overnight. The solvent was removed under reduced pressure, the residue dissolved in ether and a citrate buffer solution added. The aqueous phase was extracted with ether (3×). The combined organic phase was dried with MgSO₄, filtered, and the filtrate concentrated under reduced pressure. The residue was purified by passing through a pad of silica gel (50% EtOAc/Hex) to afford 10 g (94%) of 161-1 as a colored oil.

Step T161-2, (Based on the procedure in Meyer, S. D. and S. L. Schreiber J. Org. Chem. 1994, 59, 7549-7552.) To a solution of 161-1 (7.55 g, 40 mmol, 1.0 eq) in DCM (230 mL) was added Dess-Martin periodinane (DMP, 24 g, 56 mmol, 1.4 eq). H₂O (1.5 mL, 1.4 eq) was added with a dropping funnel to this solution over 0.5 h with vigorous stirring. After 0.5 h, Et₂O was added, the solution filtered, and the filtrate concentrated under reduced pressure. The residue was dissolved in Et₂O and the solution washed successively with saturated NaHCO₃/10% sodium thiosulfate (1:1), water and brine. Extra wash with bicarbonate-thiosulfate are sometimes needed to remove the acetic acid formed by the DMP reagent. The combined aqueous phase was back extracted with Et₂O (1×) and the combined organic phase was dried with MgSO₄, filtered, and the filtrate concentrated under reduced pressure. The residue was purified through a pad of silica gel (20% EtOAc/Hex) to give 5.4 g (75%) of 161-2 as a white solid that was gently azeotroped with toluene (3×, bath temp=30° C., oil pump) and was used immediately in the next step.

TLC: R_{f}=0.3 (hexanes/EtOAc, 1/4; detection: KMnO₄, UV).

Step T161-3. To Zn powder [activated by the following sequence: wash successively with 0.5 N HCl (3×), H₂O (3×), MeOH (3×), Et₂O (3×) and dried under vacuum (oil pump), 3.8 g, 53 mmol, 2.0 eq] and CBr₄ (19.2 g, 53 mmol, 2.0 eq) in DCM (173 mL) at 0° C. was added PPh₃ (15.2 g, 58 mmol, 2.0 eq) in three portions over 5 min, with an exothermic reaction observed. The solution was stirred at RT for 24 h The solution turned from yellow to a pink suspension. Freshly prepared aldehyde 161-2 (5.0 g, 26 mmol, 1.0 eq) was added in DCM (30 mL). The solution turns to a dark violet over the next 24 h. The solution was concentrated under reduced pressure, then purified by flash chromatography (hexanes/EtOAc, 10/1) to provide 161-3 (4.1 g, 46%) as a white solid.

TLC: R_{f}=0.67 (EtOAc/Hexanes, 3/7; detection: KMnO₄).

Step T161-4. To a solution of 161-3 (2.0 g, 5.83 mmol, 1.0 eq) in THF (distilled from Na-benzophenone ketyl, 95 mL) at −78° C. was added dropwise a freshly titrated solution of n-BuLi in hexanes (1.8 M, 10.5 mL, 17.5 mmol, 3.0 eq). The solution was stirred at −78° C. for 1.0 h. A solution of 0.01 N NaOH (100 mL) was added and the mixture warmed to RT. The aqueous phase was extracted with Et₂O (2×120 mL). The combined organic phase was washed with brine (2×300 mL), dried over MgSO₄, filtered, and concentrated under reduced pressure, then purified by flash chromatography (hexanes/EtOAc, 4/1) to give 880 mg (88%) of 161-4 as a white solid.

TLC: R_{f}=0.57 (Et₂O/Hexanes, 2/3; detection: KMnO₄).

Step T161-5. To a solution of 161-4 (880 mg, 4.81 mmol, 1.0 eq) and 161-A (see procedure for Chz-T33a, 1.65 g, 6.25 mmol, 1.3 eq) in CH₃CN (38 mL) was bubbled argon for 20 min. Et₃N (freshly distilled from CaH₂, 2.4 mL, 224 mmol, 3.6 eq) was added and argon was bubbled for 10 min. Recrystallized CuI (28 mg, 0.144 mmol, 0.03 eq) and PdCl₂(PPh₃)₂ (102 mg, 0.144 mmol, 0.03 eq) were then added to the solution. The reaction was stirred under an argon atmosphere overnight at RT. The volatiles were removed under reduced pressure and the residue purified by flash chromatography (DCM/EtOAc, 4/1) to afford 1.4 g (92%) of 161-5 as an orange solid. Note that care must be taken to remove all unreacted 161-A as it can prove very difficult to separate later.

TLC: R_{f}=0.13 (Et₂O/Hexanes, 1/4: detection: KMnO₄).

Step T161-6. To 161-5 (1.4 g, 4.39 mmol, 1.0 eq) was added 10% Pd/C (210 mg, 15% by weight) and 95% EtOH (128 mL). The mixture was placed in a Parr hydrogenator under a pressure of 400 psi of hydrogen for 24 h. The reaction was filtered through a Celite pad, then the filtrate concentrated under reduced pressure to yield 1.12 g (80%) of Boc-T161 as a colorless oil. Similarly, 29.7 g of Boc-T161a was synthesized using this procedure in 16% overall yield from 50.0 g of 134-0.

¹H NMR (CDCl₃, 300 MHz): δ 7.18-7.10 (m, 2H), 6.90-6.82 (m, 2H), 4.58-4.46 (m, 2H), 4.2-3.8 (m, 4H), 3.5 (m, 1H), 2.85-2.7 (m, 1H), 2.65-4.45 (m, 114), 1.8-1.2 (m, 4H), 1.44 (s, 9H), 0.8 (t, 3H, J=7 Hz);.

HPLC/MS (Gradient A4): t_{R}: 7.3 min, [M]⁺ 323.

The enantiomeric tethers, Boc-T161b and Boc-T177b, are accessed by the same procedure, but starting from the amino alcohol (S)-(−)-2-amino-1-butanol, 161-6, enantiomeric to 134-0.

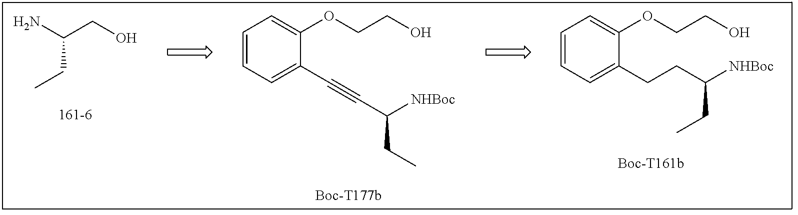

### II. Standard Procedure for the Synthesis of Tether T162

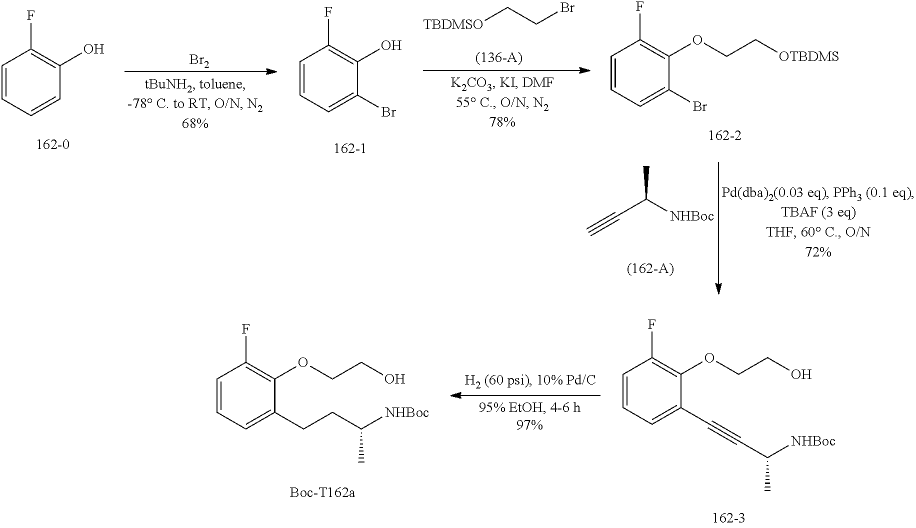

Step T162-1. To a solution of t-butylamine (43.6 g, 62.9 mL, 600 mmol, 3.0 eq) in dry toluene (170 mL) was added Br₂ (35.1 g, 11.3 mL, 220 mmol, 1.1 eq) dropwise at −30° C. (˜10 min) under N₂. The mixture was cooled to −78° C., and a solution of 2-fluorophenol (162-0, 22.5 g, 200 mmol, 1.0 eq) in DCM (110 mL) was added dropwise under N₂ (˜30 min). The mixture was warmed to RT gradually and stirred overnight. The reaction was diluted with diethyl ether and the organic phase washed with 1.0 M HCl (2×) and brine (1×). The organic phase was dried over anhydrous MgSO₄, filtered, and the filtrate evaporated under reduced pressure. The residue was purified by flash chromatography (10% EtOAc/Hex) to give the 162-1 as a brown solid (26 g, 68%).

TLC: R_{f}: 0.45 (EtOAc/Hex, 25/75; detection: UV, KMnO₄).

Step T162-2. To a solution of 162-1 (26.0 g, 136 mmol, 1.0 eq) and 136-A (52.1 g, 218 mmol, 1.6 eq) in dry DMF (500 mL) are added potassium carbonate (22.6 g, 163.2 mmol, 1.2 cq) and potassium iodide (4.5 g, 27.2 mmol, 0.2 eq). The solution was heated and stirred at 55° C. overnight under nitrogen. The mixture was diluted with water (500 mL) and diethyl ether (500 mL), and the aqueous phase extracted with Et₂O (2×300 mL). The organic phases are combined and washed with citrate buffer (400 mL) and brine (2×300 mL). The organic phase was dried over anhydrous MgSO₄, filtered, and the filtrate evaporated under reduced pressure. The yellowish oil residue was purified by flash chromatography (5% ethyl acetate/hexanes) to give 162-2 as a colorless oil (37.0 g, 78%).

TLC: R_{f}: 0.77 (EtOAc/Hex, 25/75; detection: UV, KMnO₄).

Step T162-3. A solution of 162-2 (1.05 g, 3.0 mmol, 1.0 eq), 162-A (1.02 g, 6.0 mmol, 2.0 eq), PPh₃ (79 mg, 0.3 mmol, 0.1 eq) and TBAF (1 M in THF, 9 mL, 9.0 mmol, 3.0 eq) in THF (10 mL) was degassed and refilled with argon twice. Pd₂(dba)₃ (137 mg, 0.15 mmol, 0.05 eq) was then added, the mixture degassed and refilled with argon, and the reaction stirred at 60° C. overnight under argon. The solvents were evaporated under reduced pressure and the mixture diluted with EtOAc, filtered through a silica gel pad and washed with ethyl acetate until there was no more material eluting as indicated by TLC. The solvent was removed under reduced pressure until dryness, then the residue purified by flash chromatography (40% EtOAc/Hex, repeated 2×) to yield 162-3 as an orange oil (700 mg, 72%).

TLC: R_{f}: 0.56 (EtOAc/DCM, 20/80; detection: UV, ninhydrin);

HPLC/MS (Gradient A4): t_{R}: 6.66 min, [M]⁺ 323.

Step T162-4. To a solution of 162-3 (700 mg, 2.2 mmol, 1.0 eq) in 95% ethanol (30 mL) under nitrogen was added palladium on carbon (10% by weight, 50% water, 200 mg, 30% weight eq), then treated with hydrogen gas maintained at 60 psi for 4-6 h. The reaction was filtered through a Celite pad and washed with ethanol until no additional material was eluting. The combined filtrate and washings was evaporated under reduced pressure until dryness. The residue was purified by flash chromatography (20% EtOAc/DCM) to give the Boc-T162a as a yellowish oil (690 mg, 97%).

TLC: R_{f}=0.46 (20/80, EtOAc/DCM; detection: UV, ninhydrin);

HPLC/MS (Gradient A4): t_{R}: 6.92 min, [M+H]⁺ 328;

¹H NMR (CDCl₃): δ 6.90 (m, 3H, Ar), 4.69 (br, 1H, NH), 4.15 (m, 2H), 3.93 (m, 2H), 3.67 (m, 1H), 3.07 (m, 1H, OH), 2.79 (m, 1H), 2.59 (m, 1H), 1.82-1.59 (m, 2H), 1.43 (s, 9H), 1.14 (d, J=6.5 Hz, 3H).

The enantiomeric tether, Boc-T162b, is accessed by the same procedure, but starting from the enantiomeric amino alkyne, 162-B.

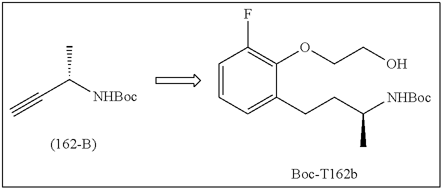

### JJ. Standard Procedure for the Synthesis of Tether T163

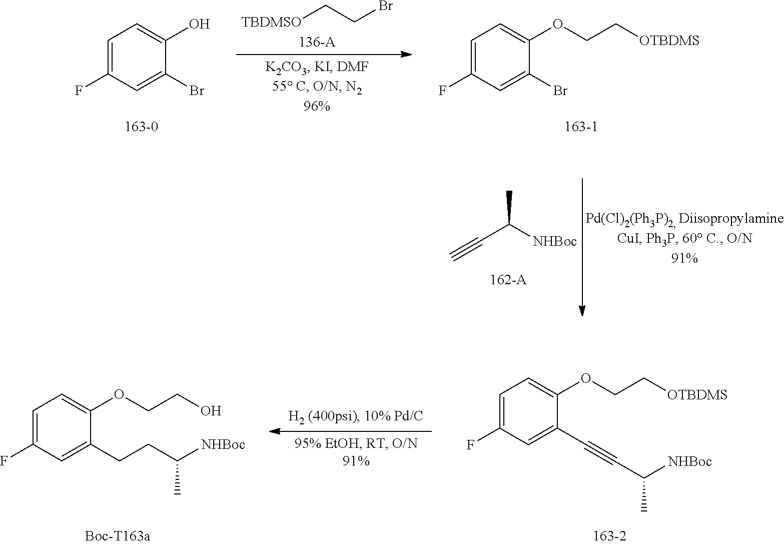

Step T163-1. To a solution of 2-bromo-4-fluorophenol (163-0, 14 g, 73 mmol, 1.0 eq) and protected 136-A (29.8 g, 125.0 mmol, 1.7 eq) in DMF (Drisolv, 230 mL) are added potassium carbonate (12.7 g, 92 mmol, 1.25 eq) and potassium iodide (2.42 g, 14.8 mmol, 0.2 eq). The reaction was heated to 55° C. and stirred overnight under nitrogen. The solvent was removed under reduced pressure until dryness, then the residual oil diluted with water (200 mL) and extracted with ether (3×150 mL). The organic phases are combined, washed with citrate buffer (2×), brine (1×), dried with magnesium sulfate, filtered, and the filtrate evaporated to dryness under reduced pressure. The residue was purified by flash chromatography (10% EtOAc/Hex) to give 163-1 as a yellowish solid (24.6 g, 96%).

TLC: R_{f}: 0.68 (EtOAc/Hex, 25/75; detection: UV, CMA);

HPLC/MS (Gradient A4): t_{R}: 13.93 min, [M+H]⁺ 349, 351.

Step T163-2. To a solution of 163-1 (3.5 g, 10 mmol, 1.0 eq), 162-A (3.0 g, 17 mmol, 1.7 eq) and triphenylphosphine (161 mg, 0.06 eq) in diisopropylamine (ACS grade, 58 mL) was bubbled argon for 15-20 min. Then, recrystallized copper (I) iodide (39 mg; 0.02 eq) and dichlorobis(triphenyphosphine) palladium (II) (210 mg, 0.03 eq) were added and the reaction mixture stirred at 60° C. overnight under argon. The solution was filtered through a silica gel pad and washed with ethyl acetate until there was additional material eluting. The solvent was removed under reduced pressure until dryness, then the residual oil purified by flash chromatography (10% EtOAc/Hex) to provide 163-2 as a yellowish oil (4.0 g, 91%).

TLC: R_{f}:0.60 (EtOAc/Hex, 25/75; detection: UV, ninhydrin);

HPLC/MS (Gradient A4): t_{R}: 13.65 min, [M]⁺ 437, [M+Na]⁺ 460.

Step T163-3. To a solution of 163-2 (4.05 g 9.41 mmol, 1.0 eq) in 95% ethanol (241 mL) under nitrogen was added palladium on carbon (434 mg, 10% by weight/50% water). (Note that more concentrated reaction conditions (>0.04 M) led to some dimer formation.) The solution was stirred under 400 psi hydrogen gas overnight. When the reaction was complete, nitrogen was bubbled through the mixture for 10 min to remove the excess hydrogen. The solvent was filtered through a Celite pad and washed with ethyl acetate until there was no additional material eluting. The combined filtrate and washings were concentrated until dryness under reduced pressure. The resulting residue was purified by flash chromatography (gradient, 30% EtOAc/Hex to 75% EtOAc/Hex) to yield Boc-T163a as a yellowish oil (2.8 g, 91%). The TBDMS group was removed during the hydrogenation.

TLC: R_{f}: 0.30 (EtOAc/Hex, 40/60; detection: UV, ninhydrin);

HPLC/MS (Gradient A4): t_{R}: 7.00 min, [M+Na]⁺ 350;

¹H NMR (CDCl₃): δ 6.84-6.75 (m, 3H), 4.6 (m, 1H), 4.01 (m, 2H), 4.0 (m, 4H), 3.65 (m, 1H), 2.7 m, 1H), 2.55 (m, 1H), 1.85 (m, 1H), 1.65 (m, 1′-1), 1.45 (s, 614), 1.15 (d, 7 Hz, 3H).

The enantiomeric tether, Boc-T163b, is accessed by the same procedure, but starting from the enantiomeric amino alkyne, 162-B.

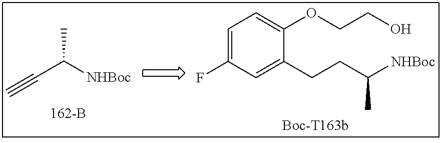

### KK. Standard Procedure for the Synthesis of Tether T164

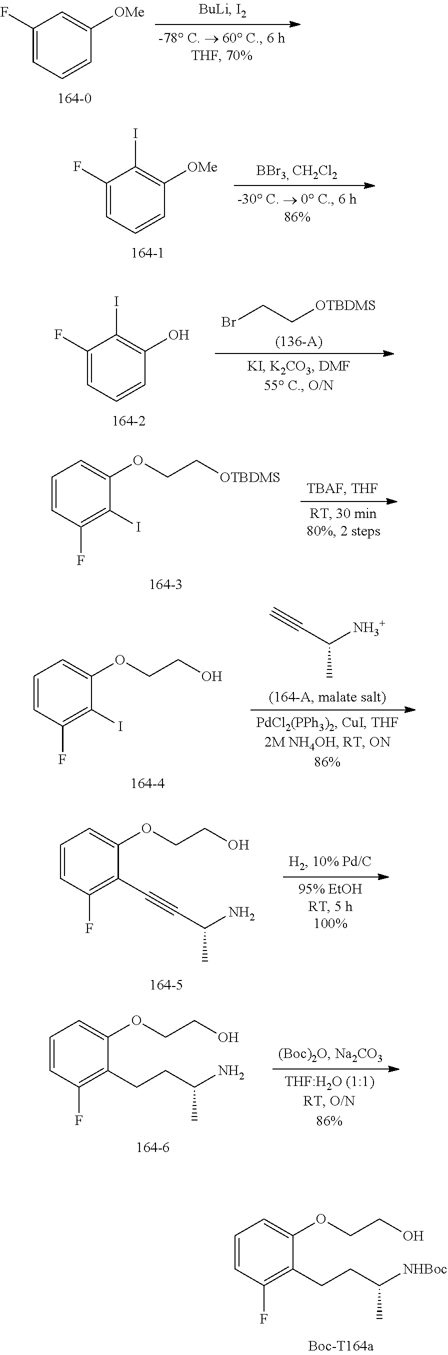

Step T164-1. To a solution of n-BuLi (36.1 mL, 1.6 M in hexanes, 57.8 mmol, 1.1 eq) in THF (dry, distilled from Na-benzophenone ketyl, 200 mL) was added a solution of 3-fluoroanisole (164-0, 6.0 mL, 52.5 mmol, 1.0 eq) in THF (dry, 20 mL) dropwise at −78° C. under N₂ (˜15 min). The reaction was stirred at −78° C. for 10 min, then a solution of I₂ (16.0 g, 63 mmol. 1.2 eq) in THF (dry, 100 mL) was added dropwise at −60-78° C. (˜30 min). The mixture was allowed to warm to −60° C. and stirred for 30 min. H₂O (50 mL) was added carefully, followed by Na₂SO₃ (10% w/v; 50 mL) and the solution stirred for 5 min. The layers were separated, the aqueous phase extracted with hexanes (3×). The combined organic phase was washed with Na₂SO₃ (10% w/v; 2×) and H₂O (2×), dried over anhydrous MgSO₄, filtered, and the filtrate concentrated under reduced pressure to leave a yellow residue, which was purified by flash chromatography (5% EtOAc/hexanes) to afford 9.3 g (70%) of 164-1 as a colorless oil.

TLC: R_{f}=0.34 (5% EtOAc/95% hexanes; detection: UV, Mo/Ce).

Step T164-2. To a solution of 164-1 (9.3 g, 36.9 mmol, 1.0 eq) in DCM (dry, 100 mL) was added a solution of BBr₃ in DCM (1.0 M, 92.3 mL, 92.3 mmol, 2.5 eq) dropwise at −30° C. under N₂ (˜30 min). The solution was allowed to warm to 0° C. over 3 h, then stirred at 0° C. for an additional 3 h. MeOH was added dropwise carefully (gas evolution), followed by the addition of H₂O. The cooling bath was removed and the mixture stirred for 10 min. The layers were separated and the aqueous phase extracted with DCM. The combined organic phase was dried over anhydrous MgSO₄, filtered, and the filtrate concentrated under reduced pressure to leave black residue, which was purified by flash chromatography (20% EtOAc/hexanes) to provide 7.5 g (86%) of 164-2 as a brown oil.

TLC: R_{f}=0.09 (5% EtOAc/95% hexanes; detection: UV, Mo/Ce).

Step T164-3. To a solution of 164-2 (7.5 g, 31.5 mmol, 1.0 eq) and 136-A (11.3 g, 47.3 mmol, 1.5 eq) in DMF (dry, 100 mL) were added K₂CO₃ (5.6 g, 41.0 mmol, 1.3 eq) and KI (1.0 g, 6.3 mmol, 0.2 eq). The mixture was stirred at 55° C. overnight. Water was added and the aqueous phase extracted with ether. The organic phase was washed with brine, dried with MgSO₄, filtered, and the filtrate concentrated under reduced pressure. The resulting residue was purified by flash chromatography (5% EtOAc/hexanes) to give 13.7 g of a mixture of the expected product 164-3 and 136-A (15% by ¹H NMR) that was used without further purification in the next step.

TLC: R_{f}=0.57 (10% EtOAc/90% hexanes; detection: UV, Mo/Ce).

Step T164-4. To a solution of 164-3 (12.8 g, 32.3 mmol, 1.0 eq) in THF (200 mL) was added a solution of TBAF (1 M in THF, 48.5 mL, 48.5 mmol, 1.5 eq) and the mixture stirred at RT for 30 min. Brine was added and the aqueous phase extracted with EtOAc. The combined organic phase was dried with MgSO₄, filtered, and the filtrate concentrated under reduced pressure. The residue was purified by flash chromatography (50% EtOAc, 50% hexanes) to yield 164-4 as a white solid (7.3 g, 80%, 2 steps).

TLC: R_{f}=0.22 (50% EtOAc/50% hexanes; detection: UV, Mo/Ce).

Step T164-5. To a solution of 164-4 (7.3 g, 1.0 eq, 25.9 mmol) in THF (52 mL) was added 164-A malate salt (5.8 g, 28.5 mmol, 1.1 eq) and the mixture degassed with Ar for 30 min. CuI (recrystallized, 248 mg, 1.3 mmol, 0.05 eq), PdCl₂(PPh₃)₂ (912 mg, 1.3 mmol, 0.05 eq) and 2 M NH₄OH in H₂O (52.0 mL, 103.6 mmol, 4.0 eq) were added and the mixture again degassed with Ar for 30 min. The reaction was stirred at RT overnight with monitoring by HPLC. The THF was evaporated and the aqueous phase acidified to pH 2 with 2 N HCl with formation of a brown insoluble gum. The aqueous phase was filtered through a small pad of Celite and rinsed with 0.01 M HCl. The aqueous phase was adjusted to pH 13-14 with basified with 6 N NaOH and extracted with EtOAc. The combined organic phase was dried with MgSO₄, filtered, and the filtrate concentrated under reduced pressure to afford 165-5 as an orange solid (5.0 g, 86%).

Step T164-6. To a solution of 164-5 (5.0 g, 22.4 mmol, 1.0 eq) in 95% EtOH (100 mL) was added wet 10% Pd/C (4.7 g, 2.24 mmol, 0.1 eq). The mixture was stirred in a Parr hydrogenator under 60 psi of H₂ for 5 h, with monitoring of the reaction by HPLC. Upon completion, nitrogen was bubbled through to remove excess hydrogen, then the mixture passed through a pad of Celite and rinsed with 95% EtOH. The combined filtrate and washings were concentrated under reduced pressure to provide 165-6 as an orange oil (5.0 g, 100%).

Step T164-7. To a solution of 165-6 (5.0 g, 22.0 mmol, 1.0 eq) in THF:H₂O (1:1, 100 mL) were added Na₂CO₃ (2.6 g, 24.2 mmol, 1.1 eq) and (Boc)₂O (5.3 g, 24.2 mmol, 1.1 eq). The mixture was stirred at RT overnight, then water added. The aqueous phase was extracted with EtOAc and the combined organic phase was dried with MgSO₄, filtered, and the filtrate concentrated under reduced pressure. The residue was purified by flash chromatography (40% EtOAc, 60% hexanes) to give Boc-T164a as a pale yellow oil (6.4 g, 86%).

TLC: R_{f}=0.47 (50% EtOAc/50% hexanes; detection: UV, Mo/Ce);

HPLC/MS (Gradient A4): t_{R}: 7.16 min, [M+Na]⁺ 350;

¹H NMR (300 MHz, CDCl₃): δ 7.10 (1H, q), 6.64 (2H, dd), 4.63 (1H broad), 413-392 (4H, m), 3.64 (1H, broad), 2.70 (2H, t), 1.80 and 1.59 (2H, 2 broad), 1.45 (9H, s), 1.15 (3H, d).

The enantiomeric tether, Boc-T164b, is accessed by the same procedure, but starting from the enantiomeric amino alkyne, 164-B.

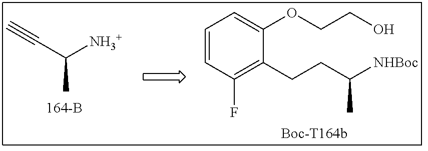

### LL. Standard Procedure for the Synthesis of Tether T165

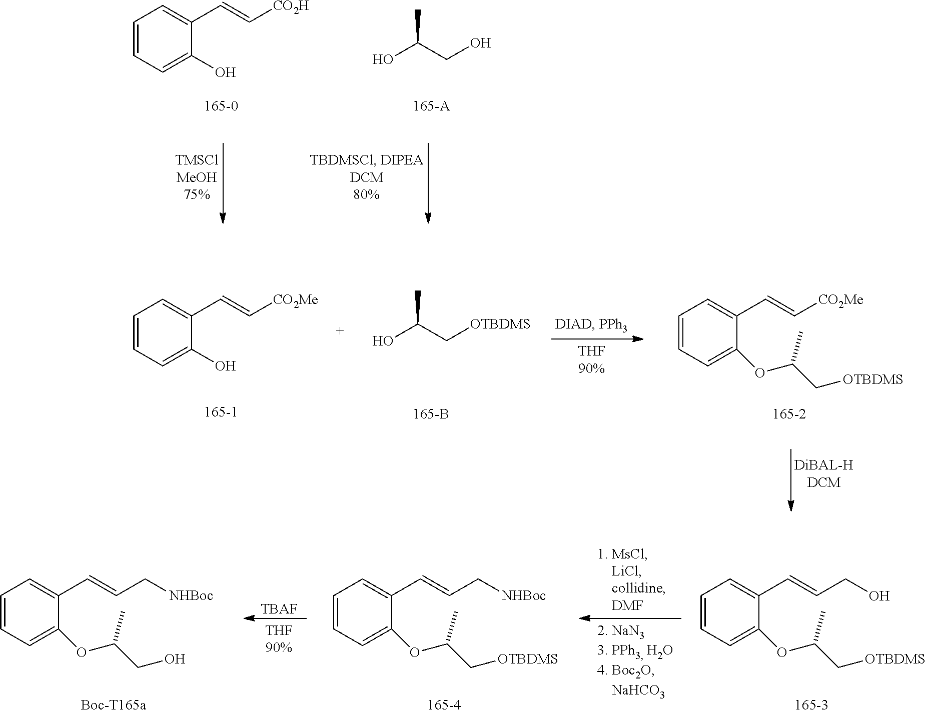

For T165a, the protected phenol 165-1 was coupled with the chiral alcohol 165-B derived from (S)-1,2-propanediol under Mitsunobu conditions to provide 165-2. Reduction of the ester to the alcohol was followed by step-wise standard transformations including conversion to the mesylate, azide displacement, reduction of the azide to the amine with triphenylphosphine, protection of the amine, and deprotection of the silyl ether to provide Boc-T165a.

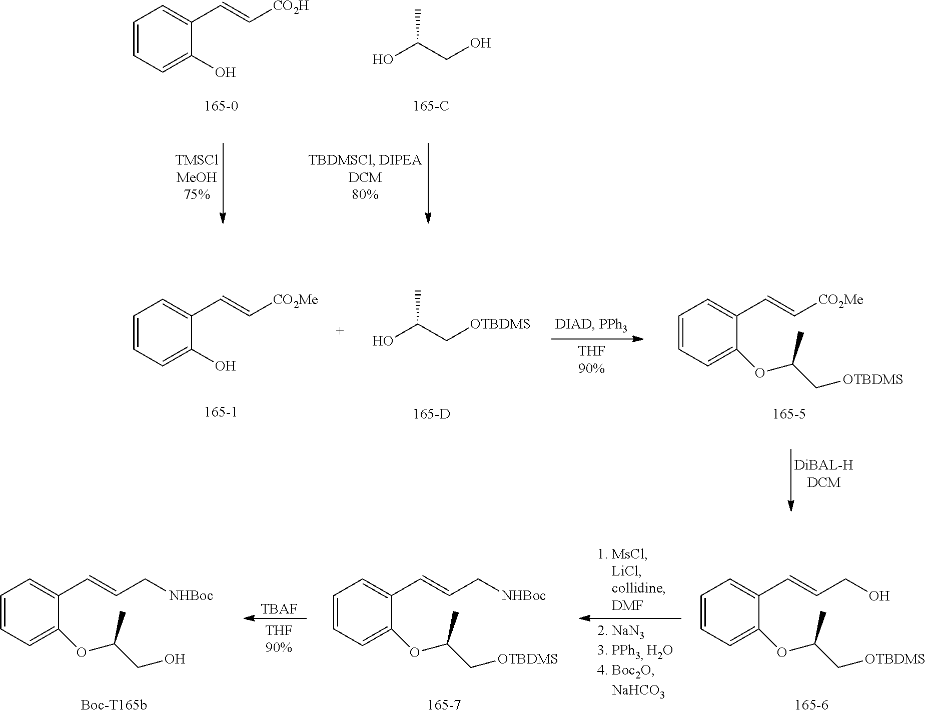

An identical sequence in equivalent yields is used to convert 165-1 to Boc-T165b except that chiral alcohol 165-D derived from (R)-1,2-propanediol was employed in the Mitsunobu reaction (to form 165-5).

### MM. Standard Procedure for the Synthesis of Tether T166

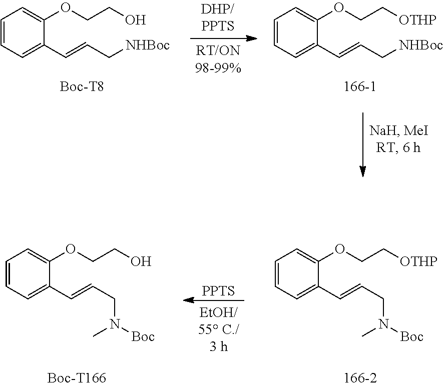

The synthesis of tether T166 was realized starting from tether T8. Protection of the alcohol as its THP ether was followed by alkylation of the carbamate nitrogen with sodium hydride as base and methyl iodide as electrophile. Acidic cleavage of the THP ether was carried out at higher temperature, but left the Boc group intact, to provide Boc-T166.

### NN. Standard Procedure for the Synthesis of Tether T167

Two alternative approaches to the synthesis of tether T167 are provided above. The first is by simple reduction of Boc-T166.

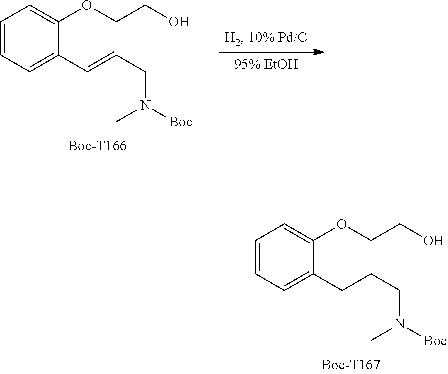

In addition, a similar sequence as described for Boc-T166 can be employed, but starting from tether T9.

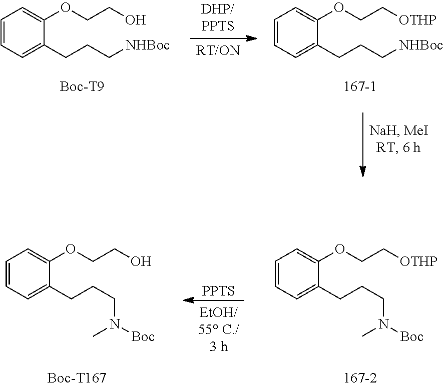

### OO. Standard Procedure for the Synthesis of Tether T168

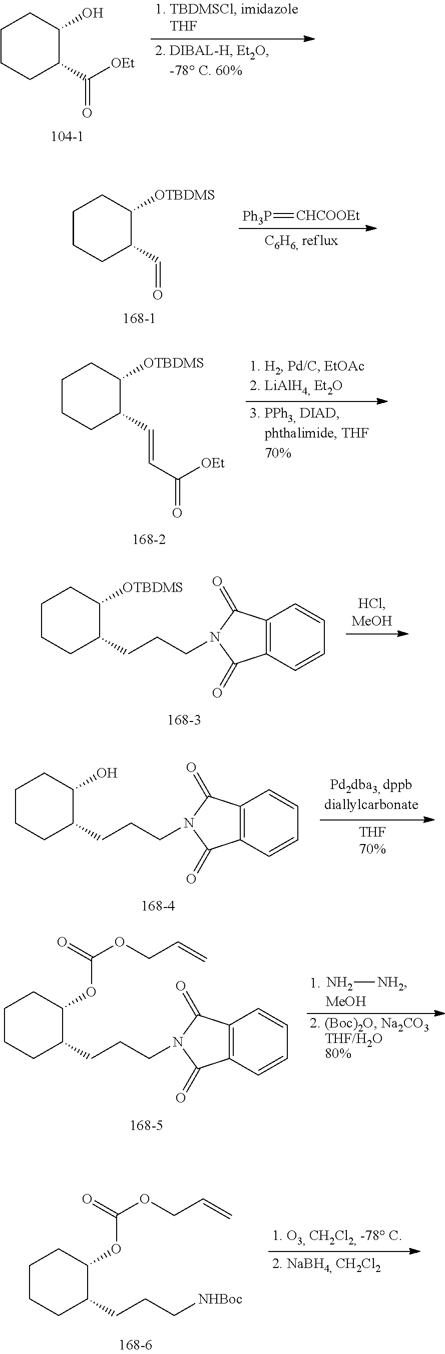

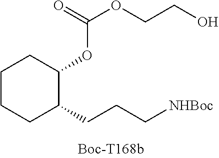

The synthesis of tether T168 was initiated from ethyl (1R,2S)-cis-2-hydroxy-cyclohexanoate 104-1 (obtained from Julich, now Codexis). Protection of the alcohol as its t-butyldimethylsilyl (TBDMS) ether was followed by controlled low temperature reduction of the ester to the corresponding aldehyde (168-1). Subsequent Wittig reaction gave the unsaturated ester 168-2. A series of transformations involving reduction of the double bond, lithium aluminum hydride reduction of the ester, and conversion of the alcohol to the corresponding phthalimido derivative via a Mitsunobu reaction produced intermediate compound 168-3 in very good yield. Deprotection of the TBDMS ether under acid conditions was followed by palladium catalyzed attachment of the allyl carbonate to afford 168-5. Cleavage of the phthalimido group with hydrazine and subsequent protection of the resulting amine as its Boc derivative provided 168-6. This intermediate was converted into Boc-T168 by ozonoloysis under reducing conditions. In addition, 168-6 could be transformed into the corresponding aldehyde, 168-7, by modification of the ozonolysis reducing conditions. 168-7 was useful in attachment of the tether by reductive amination.

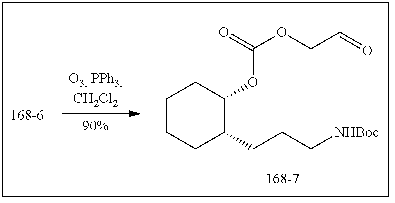

### PP. Standard Procedure for the Synthesis of Tether T169

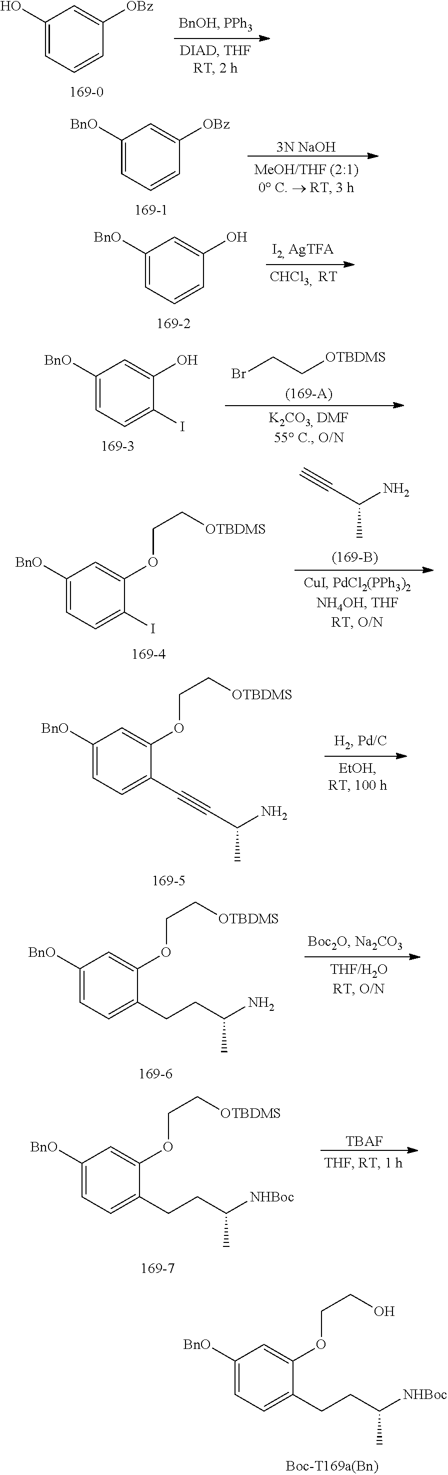

The free phenol of resorcinol monobenzoate (169-0) was protected as its benzyl ether using standard methods. Saponification of the ester gave 169-2, which was iodinated in the presence of silver trifluoroacetate to afford 169-3. Alkylation of the phenol with the protected bromide 169-A provided 169-4. In the key step, this was subjected to Pd(II) coupling with the chiral alkynyl amine 169-B yielding 169-5 possessing the entire framework of the tether. Subsequent sequential catalytic hydrogenation of the triple bond, Boc protection of the amine, and cleavage of the TBDMS ether were conducted with standard methods to leave Boc-T169a(Bn). Use of the enantiomeric amine of 169-B provided a route to the enantiomeric tether Boc-T169b(Bn).

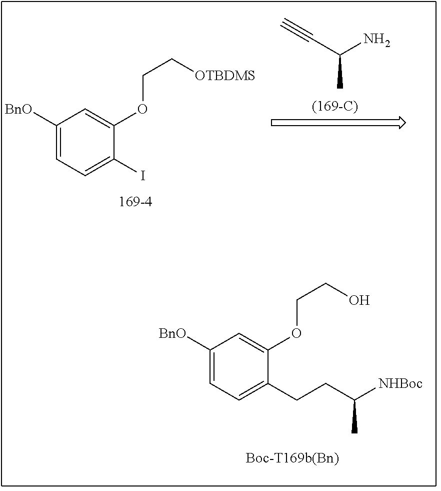

### QQ. Standard Procedure for the Synthesis of Tether T170

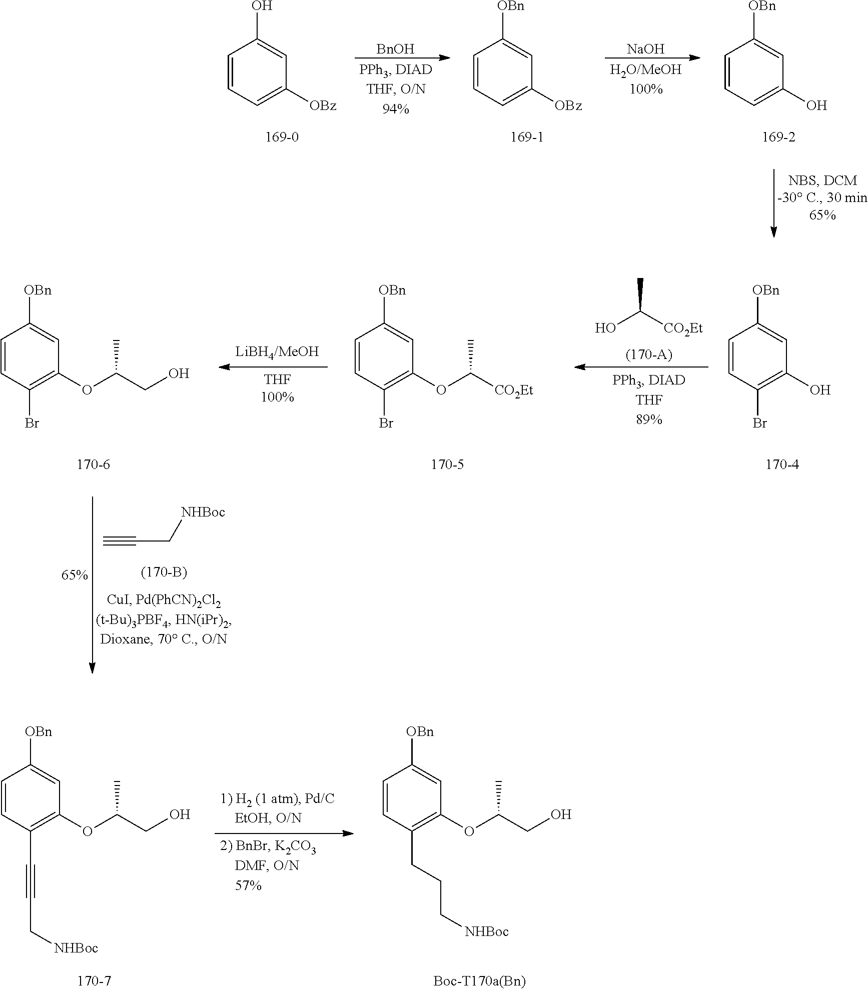

Starting from 30 g (0.14 mol) of resorcinol monobenzoate (169-0), the free phenol was protected as its benzyl ether utilizing standard methodology. Cleavage of the ester in base followed by bromination with NBS gave the 4-bromoderivative (170-4). Mitsunobu coupling with (S)-ethyl lactate (170-A) provided 170-5. The ester was reduced with lithium borohydridc and the resulting bromoalcohol (170-6) subjected to Pd(II)-mediated coupling with Boc-propargylamine (170-B). The alkyne was reduced to 170-7, with concomitant cleavage of the benzyl ether, which protection then had to be restored under standard conditions to yield the protected tether derivative. Alternatively, 170-6 could be subjected to a different Pd(II)-mediated coupling reaction with Boc-allylamine (170-C) to provide the protected tether directly. Use of (R)-ethyl lactate (or other appropriate alkyl ester of (R)-lactic acid) in this procedure provides the corresponding protected enantiomeric tether Boc-T170b(Bn).

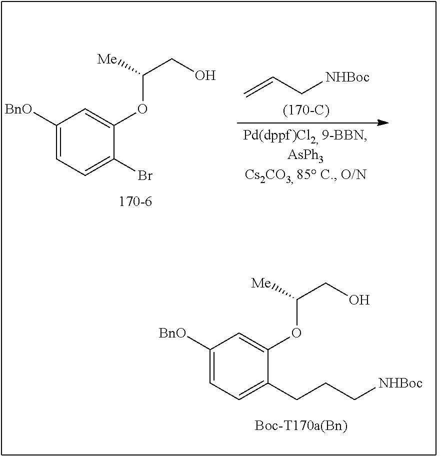

### RR. Standard Procedure for the Synthesis of Tether T171

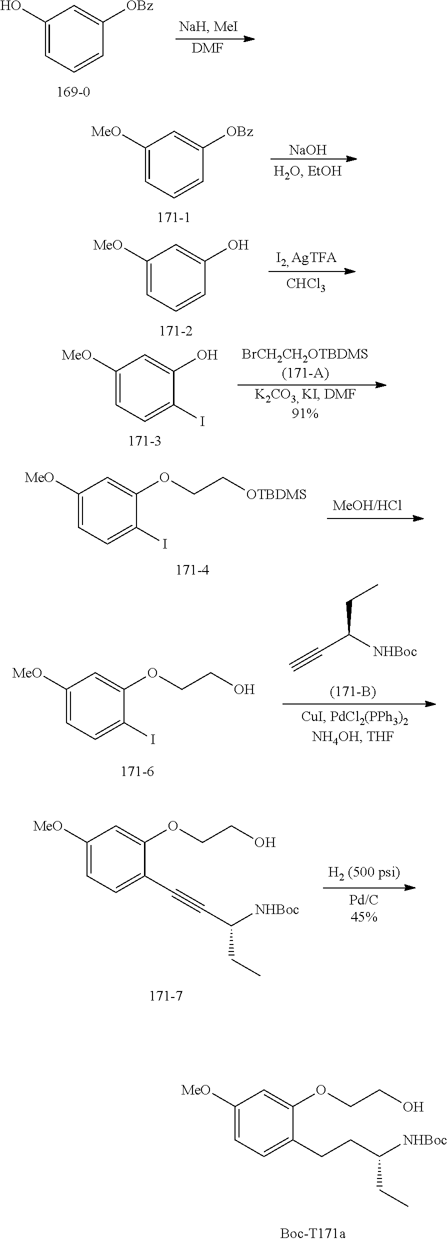

The synthesis of tether T171a proceeded as presented above starting from the monobenzoate of resorcinol (169-0). Protecting group manipulation followed by iodination gave 171-3. Alkylation with 171-A (equivalent to 134-0, see synthesis described with T161) followed by Sonogashira coupling with 171-B gave intermediate 171-7. Reduction provided Boc-T171a, The enantiomeric tether T171b, is accessed using the same sequence, but using 171-C (equivalent to alkyne derived from 161-6, see synthesis described with T161), the enantiomeric reagent of 171-B.

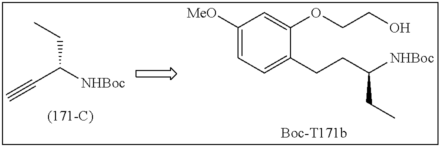

### SS. Standard Procedure for the Synthesis of Tether T172

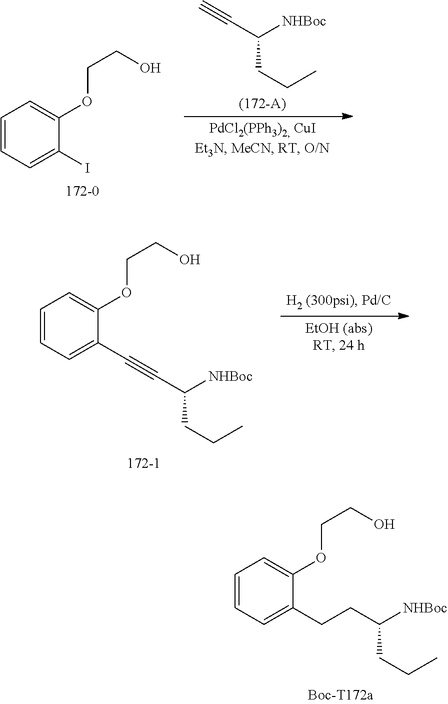

The synthesis of tether T172a proceeded starting from protected iodo-phenol 172-0 and a Pd(0)-mediated Sonagashira coupling with the protected amino alkyne, 172-A, to yield 172-1. Reduction of the alkyne provided Boc-T172a.

The chiral reagent 172-A is accessed as illustrated originating from (R)-2-amino-1-pentanol (172-2).

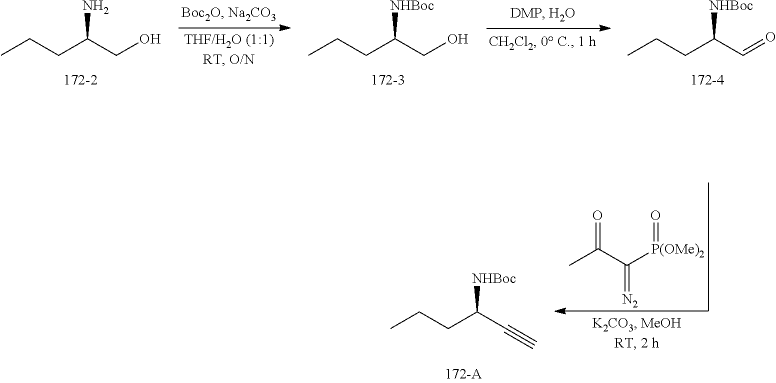

The enantiomeric tether, T172b, is constructed similarly, but using the reagent 172-B, which is synthesized as outlined for 172-A beginning from (S)-2-amino-1-pentanol.

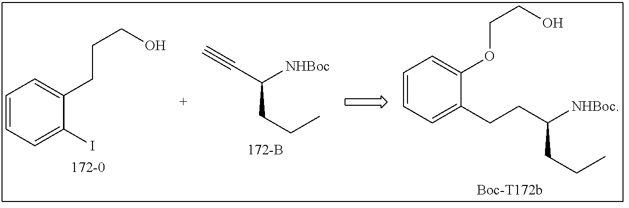

### TT. Standard Procedure for the Synthesis of Tether T173

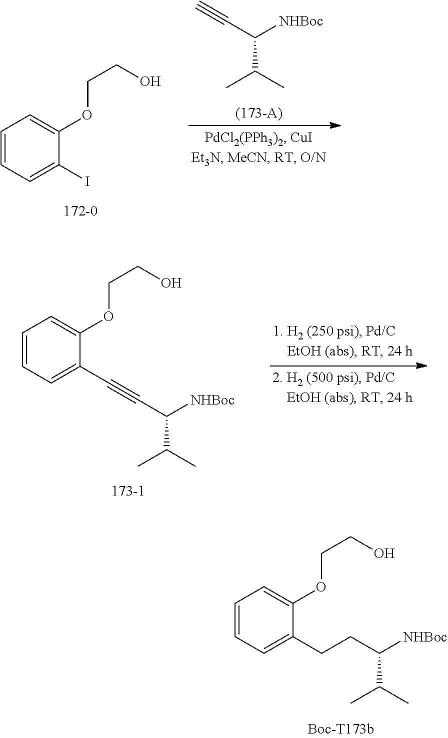

In a similar manner to that just described for T172, the preparation of tether T173b started from protected iodo-phenol 172-0 and a Pd(0)-mediated Sonagashira coupling with the protected amino alkyne, 173-A, to yield 173-1, followed by complete reduction of the alkyne yielded Boc-T173b. The 173-A reagent is accessed from the chiral amino alcohol, 173-0, as shown.

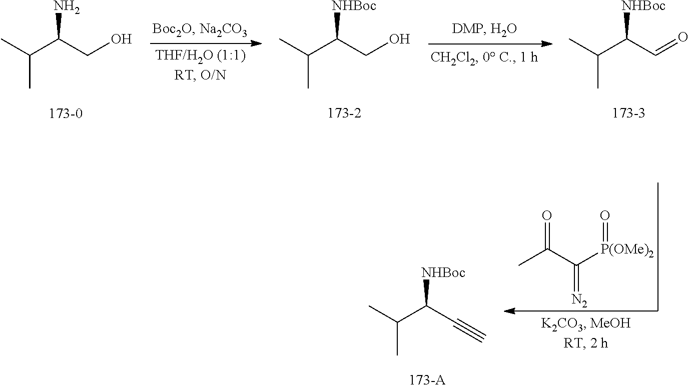

The enantiomeric tether Boc-T173a is constructed using the same process utilizing the reagent 173-B, which in turn can be synthesized from the enantiomeric amino alcohol 173-4 as described for 173-A.

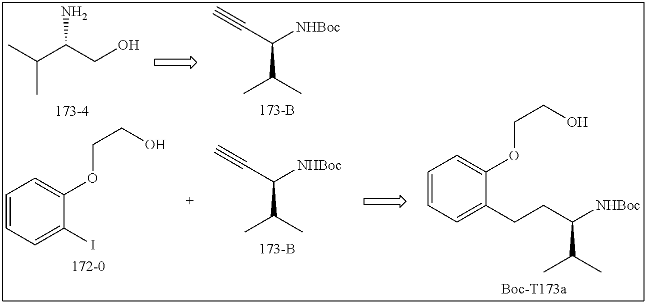

### UU. Standard Procedure for the Synthesis of Tethers T174 and T175

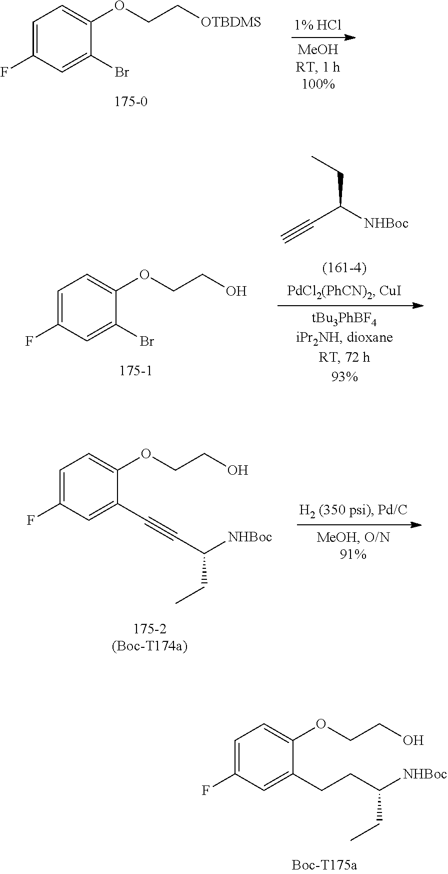

Tethers T174 and T175 are accessed from the same sequence starting from the alkylated phenol (175-0) prepared in a manner similar to the synthons already described. Deprotection followed by Sonogashira coupling with the chiral alkyne, 161-4, gave 175-2 in high yield, which is equivalent to Boc-T174a. Reduction of 175-2 then provided Boc-T175a also in excellent yield.

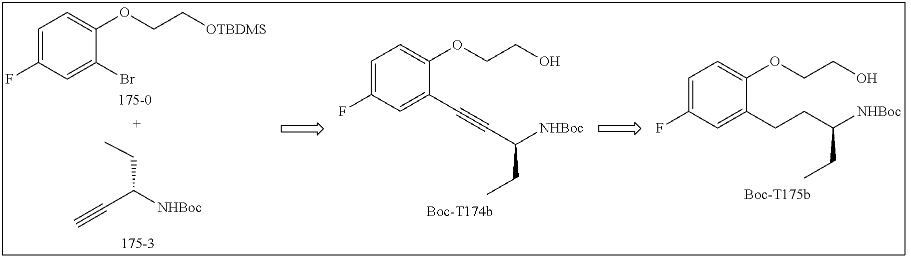

The enantiomeric tethers T174b and T175b are prepared employing an identical sequence using 175-3, the enantiomeric reagent to 161-4.

### VV. Standard Procedure for the Synthesis of Tether T176

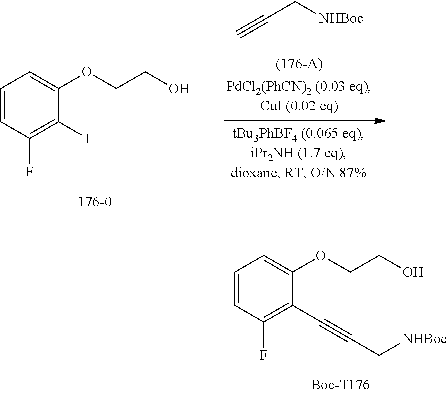

In a straightforward manner, Sonogashira coupling of the alcohol 176-0 with Boc-protected propargylamine (176-A) yielded Boc-T176. 176-0 can be accessed from the corresponding phenol by a two-step sequence involving alkylation with a protected 2-haloalcohol followed by deprotection.

### WW. Standard Procedure for the Synthesis of Tethers T178 and T179

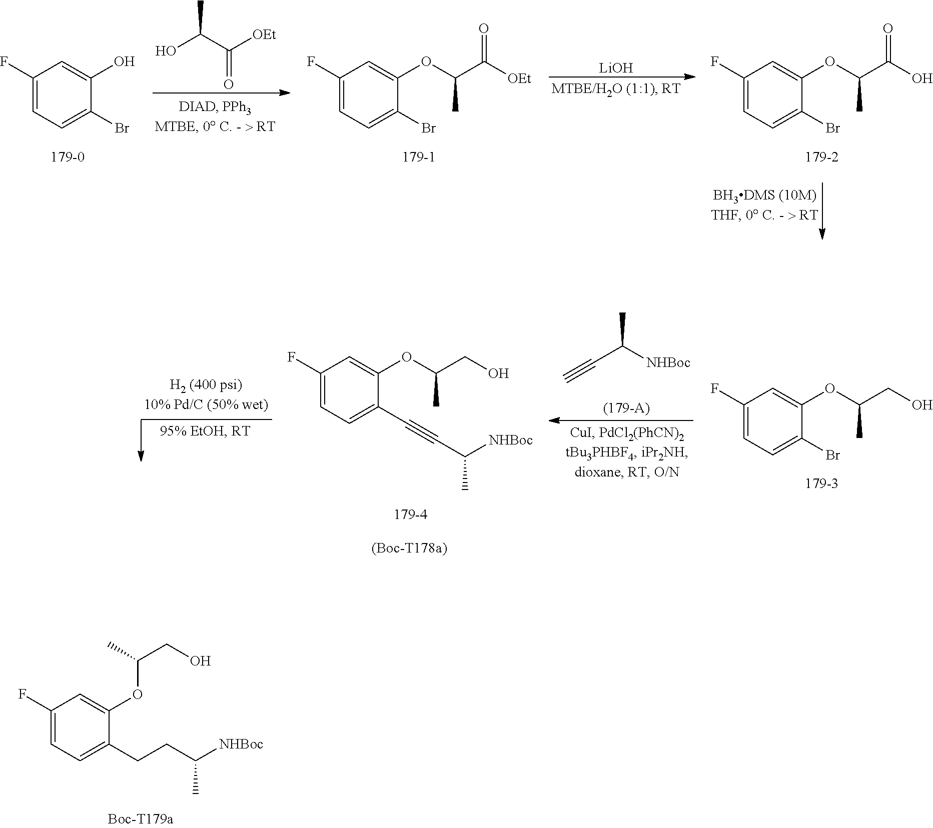

The tethers T178 and T179 both are generated from the single sequence illustrated above. Mitsunobu reaction of the halogenated phenol (179-0) with (S)-ethyl lactate gave 179-1. Hydrolysis to 179-2, followed by borane reduction provided the bromide 179-3, as the precursor to the Pd(0)-coupling reaction. Sonogashira of this intermediate using the chiral alkynylamine (179-A) gave 179-4, which is equivalent to Boc-T178a. Complete reduction of the triple bond then produced Boc-T179a.

An analogous method, but using the enantiomeric alkyne, 179-B, provides the protected tethers, Boc-T178 h and Boc-T179b. Similar methods, but utilizing (R)-ethyl lactate or other appropriate (R)-lactate ester, are used to provide the diastereomeric tethers Boc-T178c, Boc-T178d, Boc-T179c and Boc-T179d.

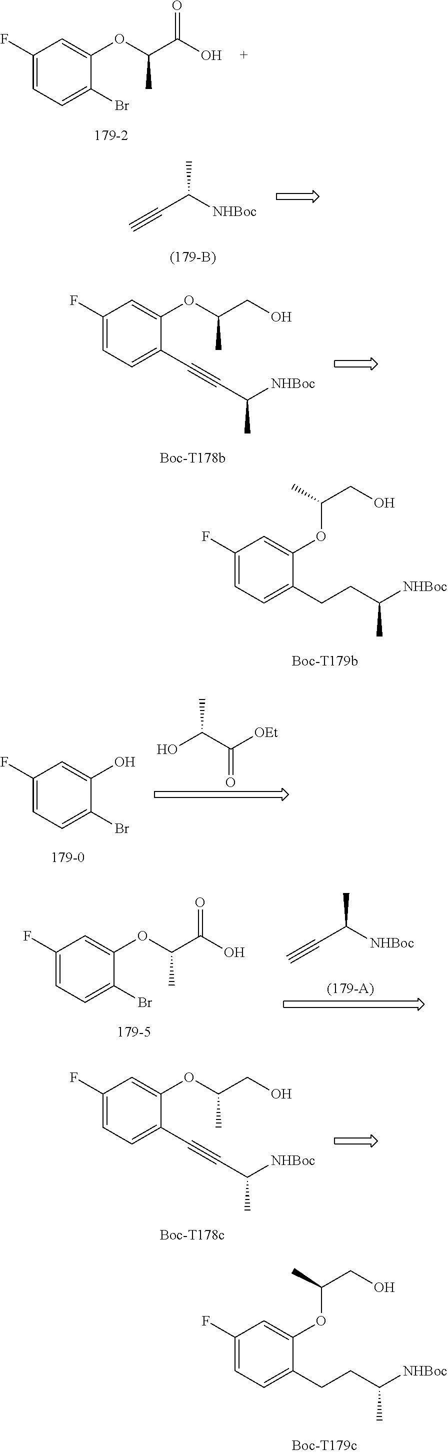

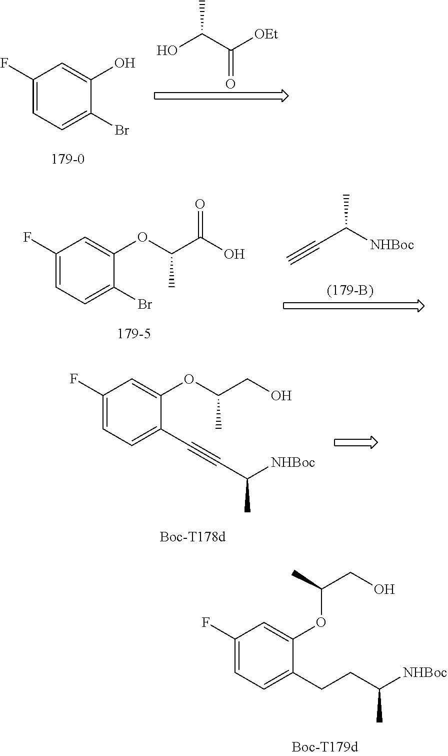

### YY. Standard Procedure for the Synthesis of Tethers T180 and T181

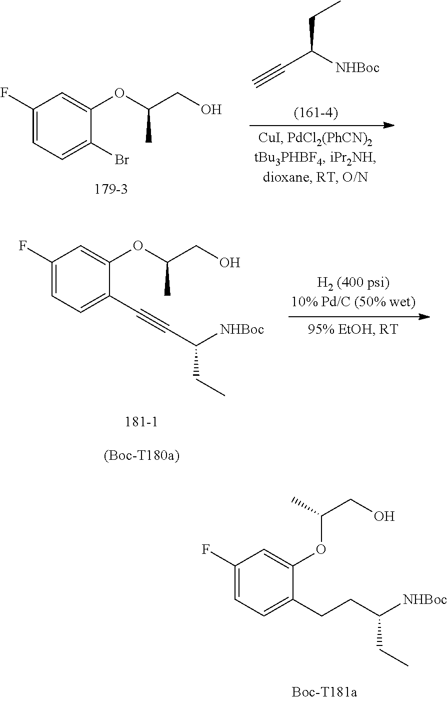

Beginning from intermediate 179-3, tethers T180 and T181 are prepared by Sonogashira coupling with the protected alkynylamine 161-4 followed by reduction of the coupled product 181-1 (equivalent to Boc-T180a) to provide Boc-T181a.

The diastereomeric tethers, Boc-T180b and Boc-T181b, are accessed by the same procedure, but using 175-3, the enantiomeric reagent to 161-4. Employing 179-6, the enantiomer of 179-3, together with 161-4 or 175-3, can be used to synthesize Boc-T180c and Boc-T181c or Boc-T180d and Boc-T181d, respectively.

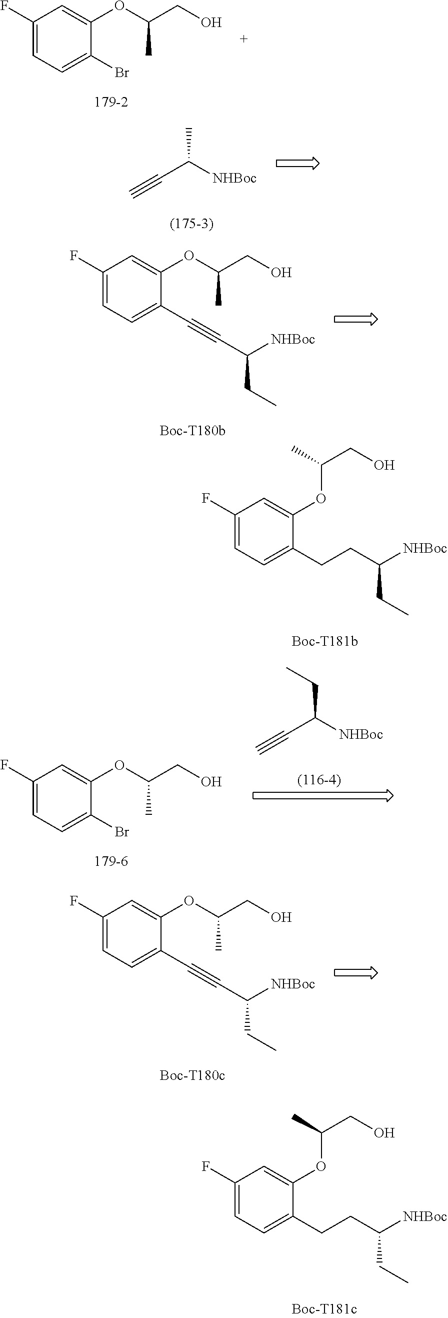

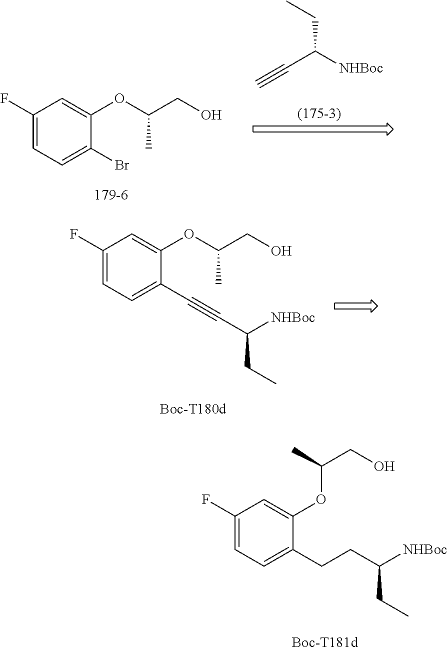

### ZZ. Standard Procedure for the Synthesis of Tethers T182 and T183

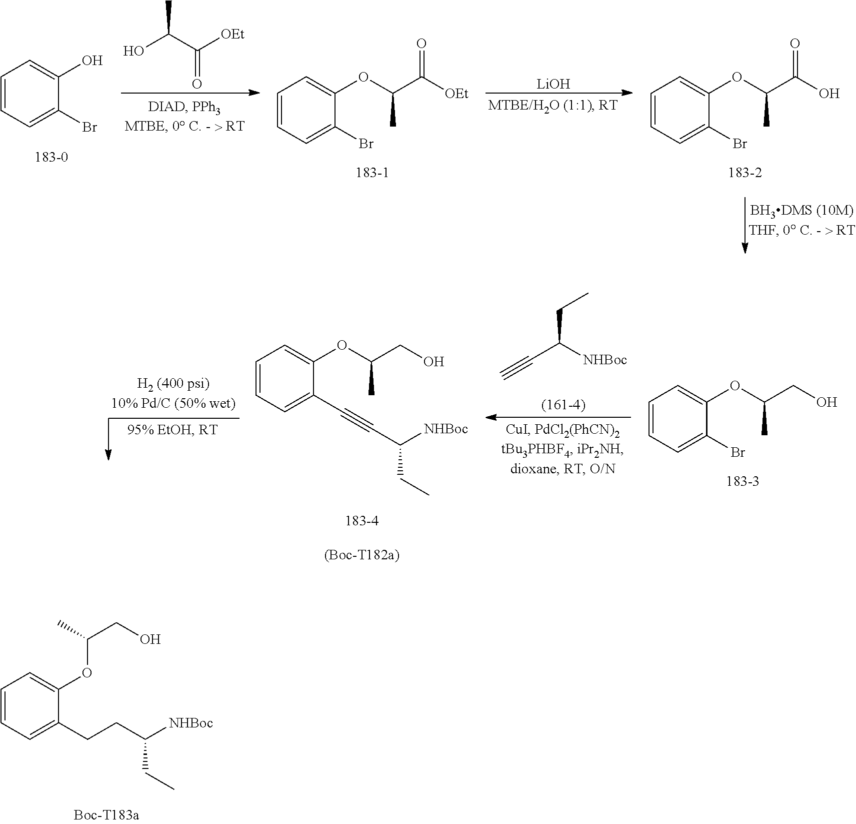

Alkylation of the bromophenol 183-0 with (S)-ethyl lactate under Mitsunohu conditions is used to synthesize 183-1. Base hydrolysis followed by borane reduction gives the intermediate alcohol 183-3. Sonogashira coupling with the alkynylamine 161-4 yields 183-4, equivalent to Boc-T182a. Complete reduction of the triple bond then provides Boc-T183a. The diastereomeric tethers, Boc-T182b and Boc-T183b, are accessed by a similar procedure, but using 175-3, the enantiomeric reagent to 161-4. Employing 183-5, the enantiomer of 183-3, together with 161-4 or 175-3, can be used to synthesize Boc-T182c and Boc-T183c or Boc-T182d and Boc-T183d, respectively. 183-5 can be prepared from (R)-ethyl lactate or another suitable ester.

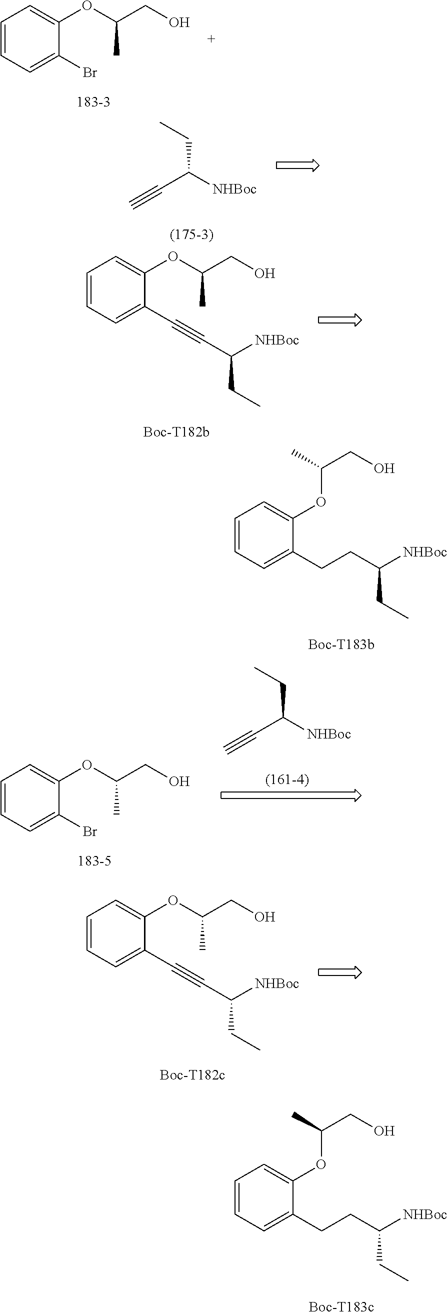

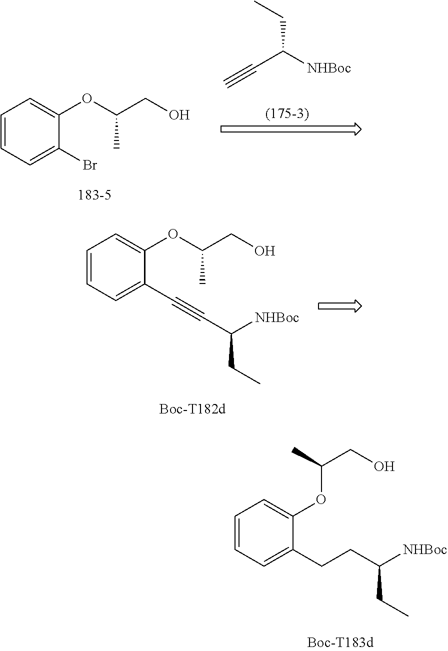

### AAA. Standard Procedure for the Synthesis of Tethers T184 and Tether T185

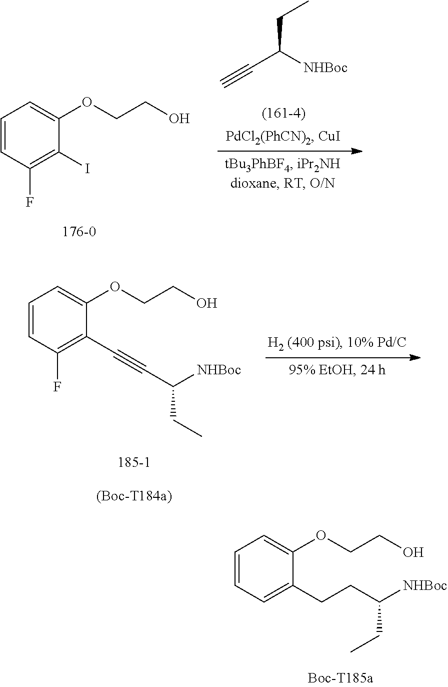

In a straightforward manner starting from intermediate 176-O, Sonogashira coupling with 161-4 gives 185-1 (equivalent to Boc-T184a). Reduction of the alkyne then provides Boc-T185a.

The enantiomeric tethers, Boc-T184b and Boc-T185b, can be accessed by the same procedures, but using 175-3, the enantiomeric reagent to 161-4.

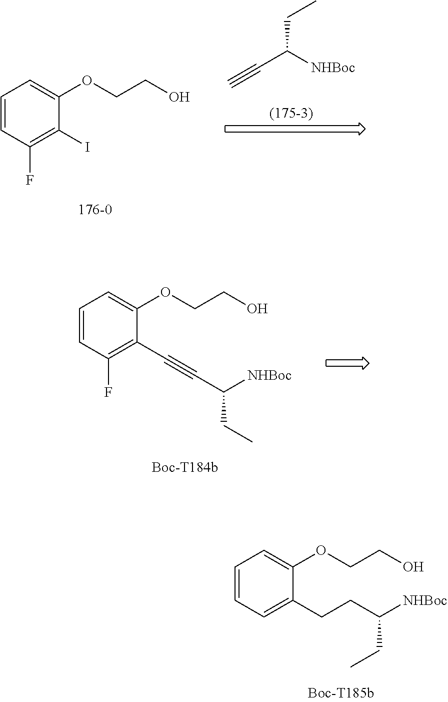

### BBB. Standard Procedure for the Synthesis of Tether T186

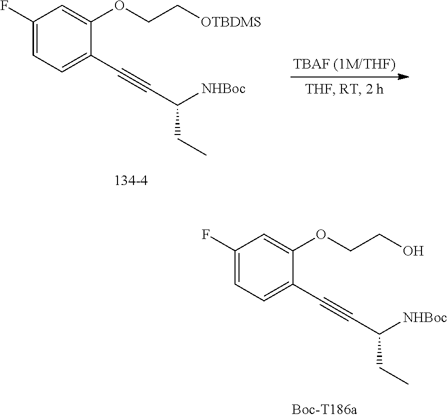

Deprotection of intermediate 134-4 under standard conditions is used to provide Boc-T186a. The enantiomer of 134-4 leads to the enantiomeric tether Boc-T186b.

### CCC. Standard Procedure for the Synthesis of Tether T187

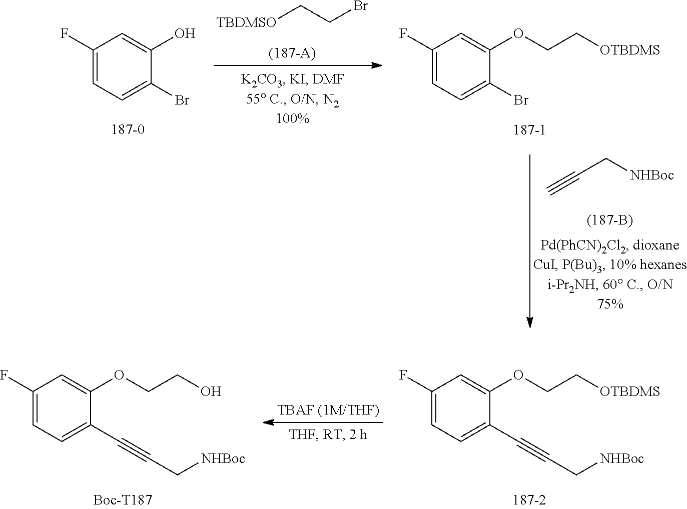

The dihalogenated phenol, 187-0, was alkylated with the protected bromo alcohol, 187-A, then subjected to Pd(0)-coupling conditions to prepare the intermediate 187-2 in very good yields. Deprotection utilizing standard methods gave Boc-T187.

### DDD. Standard Procedure for the Synthesis of Tether T188

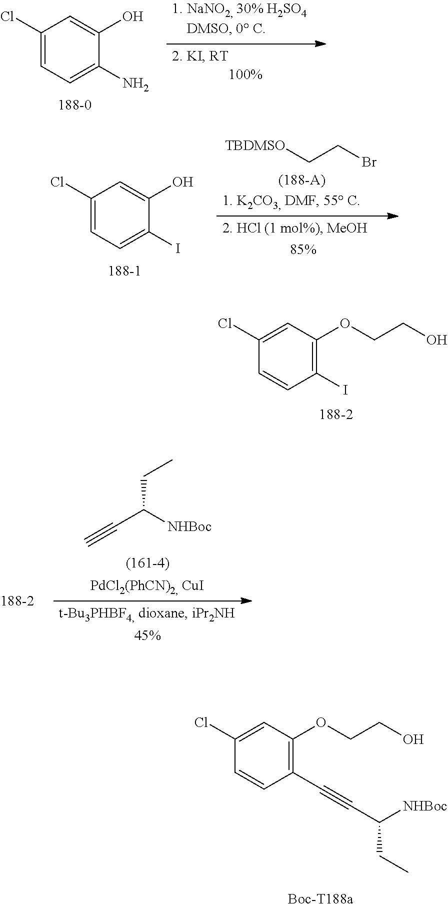

The iodophenol, 188-1, was prepared through a diazotization-displacement sequence. Alkylation with the protected bromoalcohol 188-A, followed by hydrolytic removal of the silyl ether protecting group left 188-2. Sonogashira coupling with chiral alkynylamine 161-4 prepared Boc-T188a in modest yield. An alternative, one step sequence, was also effective for providing 188-2 directly from 188-0.

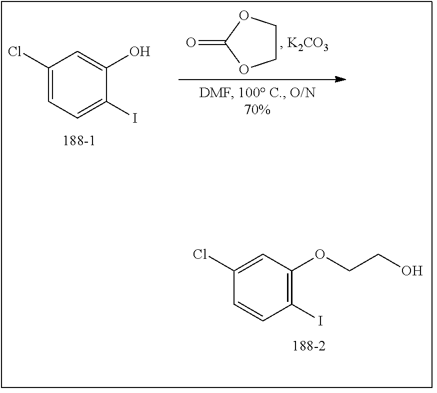

The enantiomeric tether, Boc-T188b, can be synthesized by the same procedure, but using 175-3, the enantiomeric reagent to 161-4.

### EEE. Standard Procedure for the Synthesis of Tether T189

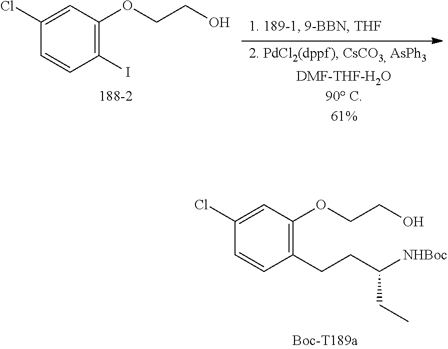

A B-Alkyl Suzuki-Miyaura coupling of intermediate iodoalcohol 188-2 with the alkene 189-1 was utilized to prepare the protected tether Boc-T189a. The reagent 189-1 was provided by partial reduction of the alkyne, 161-4.

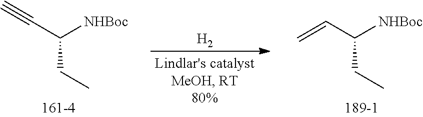

175-3, the enantiomer of 161-4, likewise can be used to provide 189-2. This, when subjected to the Pd(0)-conditions just described leads to the enantiomeric tether Boc-T189b.

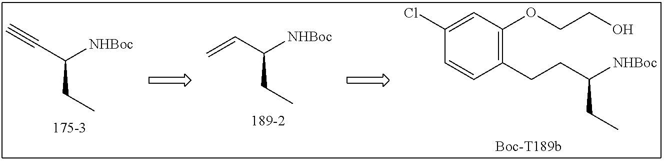

### FFF. Standard Procedure for the Synthesis of Tether T190

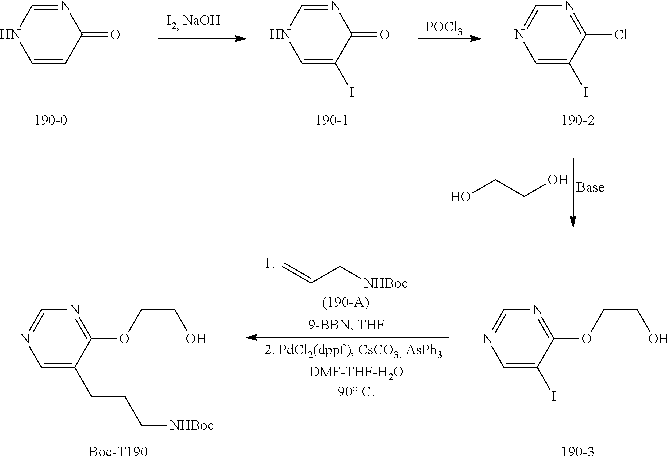

Iodination of 190-0, followed by chlorination and displacement with the alkoxide from ethylene glycol, gives 190-3. B-Alkyl Suzuki-Miyaura coupling using protected allylamine 190-A leads to Boc-T190.

### GGG. Standard Procedure for the Synthesis of Tether T191

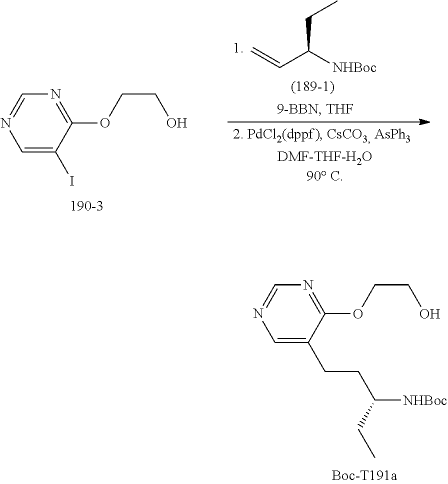

Modification of the alkene component in the process described for tether T190 is used to access tether T191. Substitution of the protected chiral unsaturated amine 189-1 in the B-alkyl Suzuki-Miyaura reaction provides Boc-T191a. Analogously, 189-2, the enantiomer of 189-1, can be used to prepare the enantiomeric tether Boc-T191b.

### HHH. Standard Procedure for the Synthesis of Tether T192

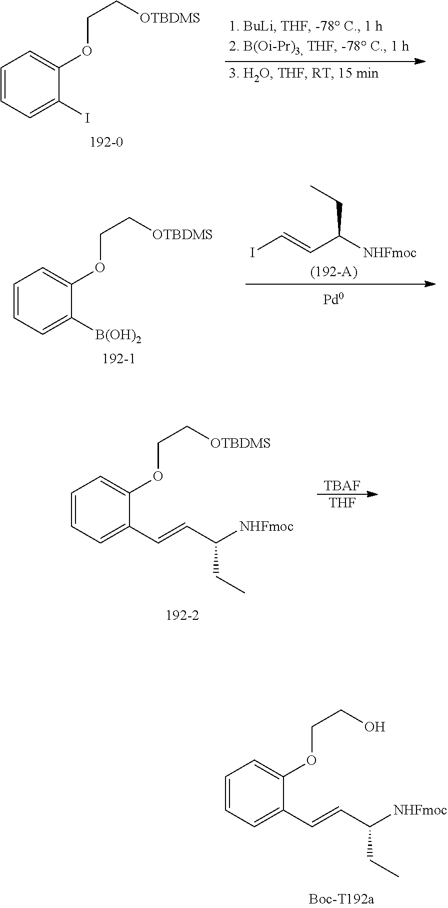

The boronic acid, 192-1, is synthesized from the iodide, 192-0, by a multi-step process involving metal-halogen exchange, treatment with triisopropylborate and hydrolysis. Suzuki coupling with the chiral iodide gives 192-2, which is then deprotected to leave Boc-T192a. The enantiomer of 192-A can be employed to provide the enantiomeric tether, T192b.

### III. Standard Procedure for the Synthesis of Tether T193

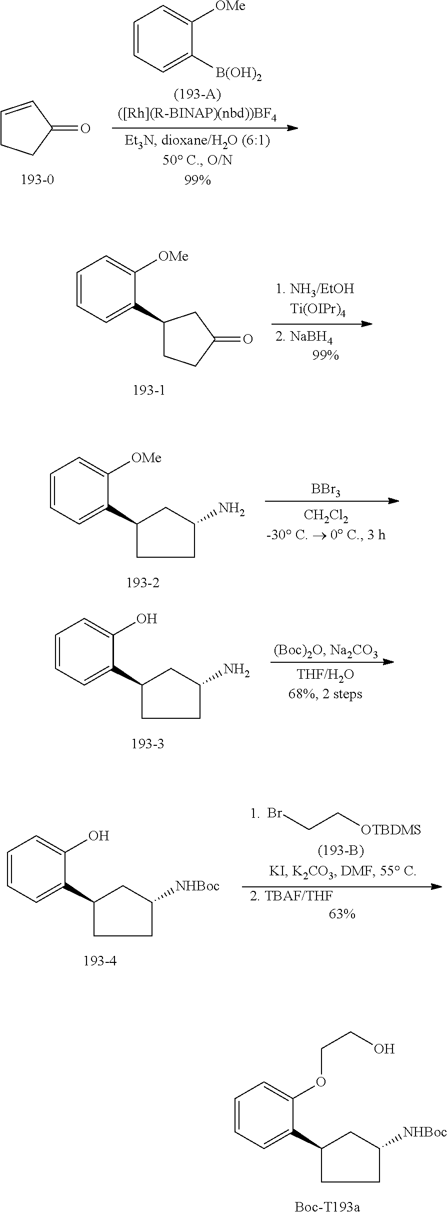

Cyclopentenone (193-0) is reacted with the boronic acid 193-A in the presence of the chiral rhodium complex indicated to provide 193-1 in good optical purity (>96% ee). Reductive amination, cleavage of the aromatic methyl ether and protection of the amine gives 193-4. Alkylation of the phenol with the protected synthon 193-B and deprotection of the silyl ether leads to Boc-T193a. Use of the S-BINAP ruthenium complex would produce 193-5, the enantiomeric cyclopentanone to 193-1, which in turn provides Boc-T193b.

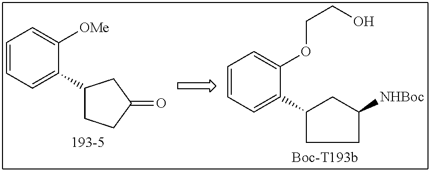

### JJJ. Standard Procedure for the Synthesis of Tether T194

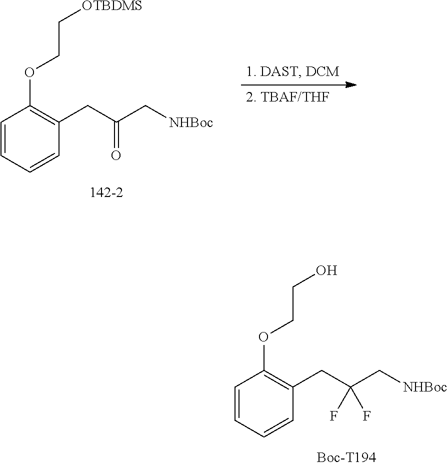

Boc-T194 is synthesized from the ketone derivative 142-2, an intermediate in the construction of T142, by treatment with DAST, followed by treatment with TBAF to ensure complete deprotection of the TBDMS ether.

### KKK. Standard Procedure for the Synthesis of Tether T195

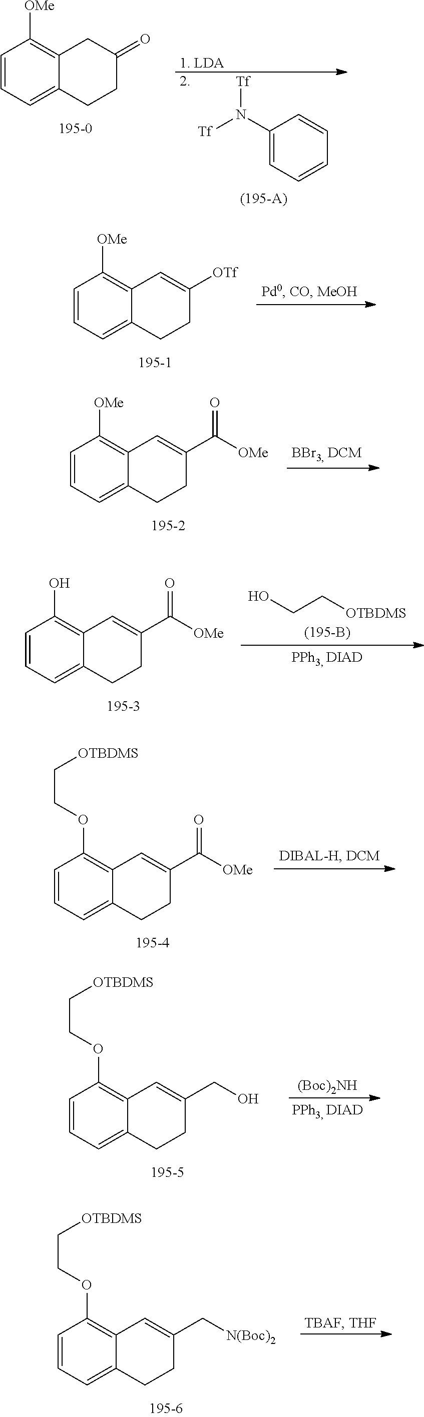

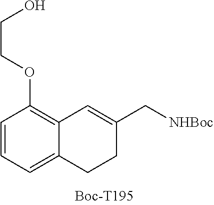

Formation of the alkenyl triflate 195-1 from 195-0 is performed in a standard manner. Palladium-catalyzed carbonylation is followed by methyl ether deprotection to give 195-3. Mitsunobu reaction of the phenol with the mono-t-butyldimethylsilylether of ethylene glycol (195-B) yields 195-4. Reduction of the ester to the alcohol leads to 195-5, which is then converted into the diprotected amine 195-6 again using a Mitsunobu process. The synthesis of Boc-T195 is completed by deprotection of the silyl protecting group with fluoride.

### LLL. Standard Procedure for the Synthesis of Tether T197

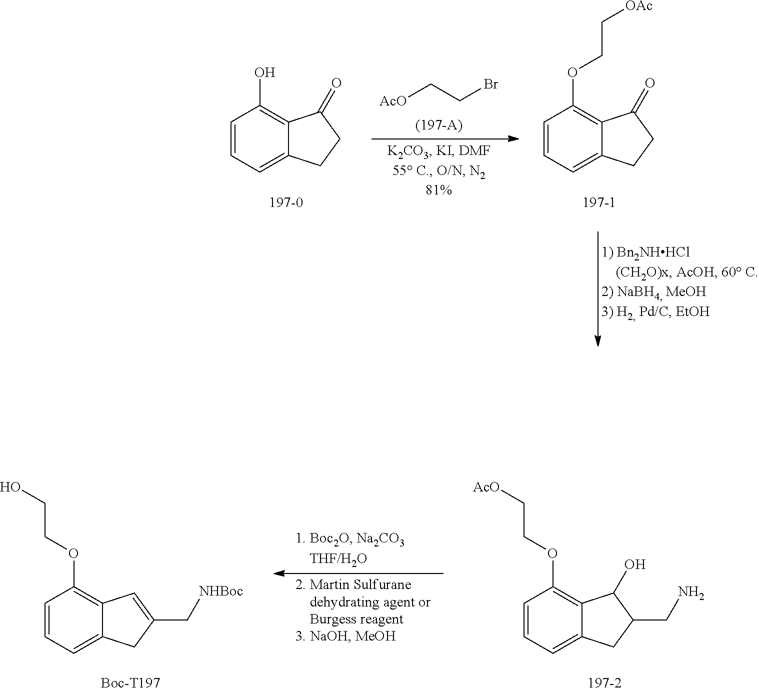

Alkylation of 197-0 proceeds well to give the ketone, 197-1. Concomitant aminomethylation and reduction of the carbonyl occurs under the reducing conditions indicated to prepare 197-2. Protection of the amine, dehydration and acetate hydrolysis results in Boc-T197.

### MMM. Standard Procedure for the Synthesis of Tether T198

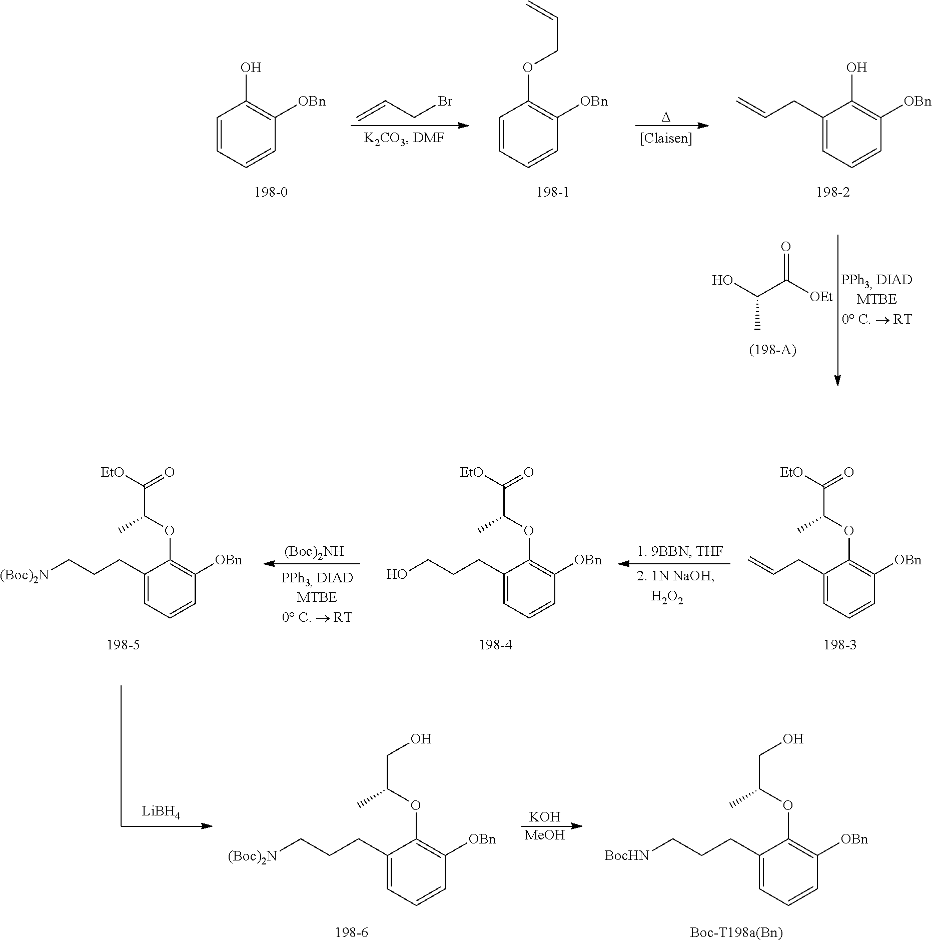

This tether is constructed beginning with protection of 2-benzyloxyphenol (198-0) as an allyl ether followed by Claisen rearrangement to provide 198-2. Mitsunobu reaction with (S)-ethyl lactate (199-A) gave 198-3. Hydroboration of the double bond and subsequent oxidation yielded 198-4. Another Mitsunobu reaction, this time with di-t-butyliminodicarboxylate gave 198-5. Reduction of the ester with lithium borohydride and base cleavage of one of the Boc groups succeeded in affording Boc-T198a(Bn). Use of (R)-ethyl lactate (or other appropriate alkyl ester of (R)-lactic acid) in this procedure provides the corresponding protected enantiomeric tether Boc-T198b(Bn).

### NNN. Standard Procedure for the Synthesis of Tether T199 Boc-(2RMe,5OH)o18r

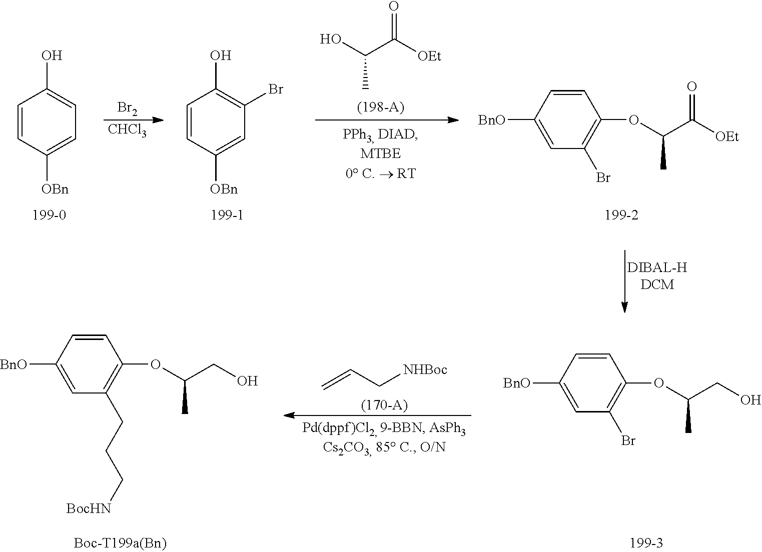

In a manner analogous to that already described for T170, this tether was constructed starting from commercially available 4-(benzyloxy)phenol (199-0). This was brominated to give the 2-bromo derivative (199-1), which was coupled to (S)-ethyl lactate (199-A) under Mitsunobu conditions to provide 199-2. The ester was reduced to the alcohol with DIBAL-H to afford 199-3. Suzuki coupling to the 9-BBN derivative of 170-A yielded the protected tether, Boc-T199a(Bn). Use of (R)-ethyl lactate (or other appropriate alkyl ester of (R)-lactic acid) in this procedure provides the corresponding protected enantiomeric tether Boc-T199b(Bn).

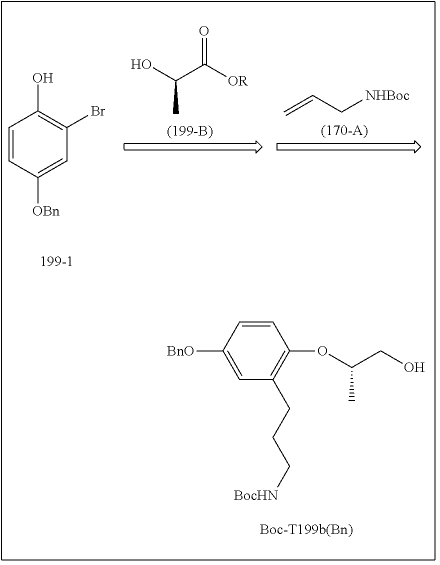

### OOO. Standard Procedure for the Synthesis of Tether T200

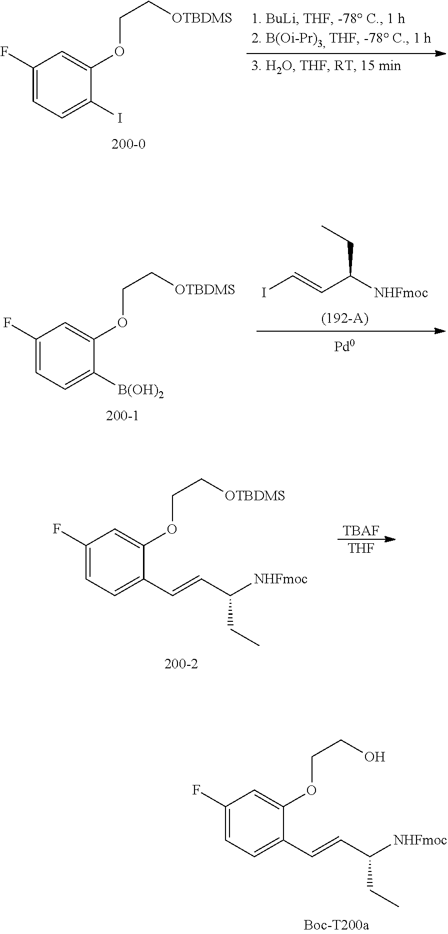

Similar to the process described for tether 192, halogen-metal exchange of the iodide 200-0, reaction with triisopropylhorate and hydrolysis leads to the boronic acid, 200-1. Suzuki coupling with the chiral alkenyl iodide 192-A and silyl deprotection yields Boc-T200a. Alternatively, the tin reagent 192-B or its enantiomer can be employed in the route to this tether.

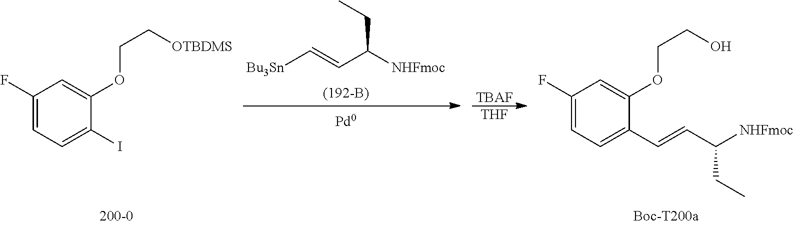

Use of 192-C, the enantiomer of 192-A, provides the enantiomeric tether, Boc-T200b.

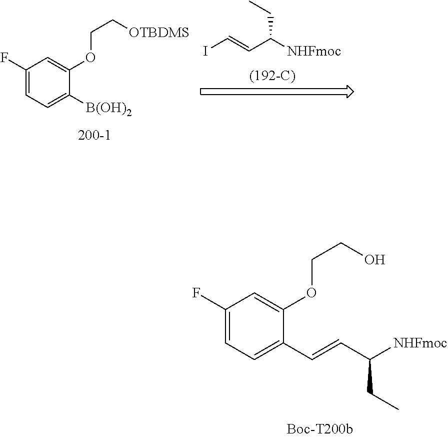

### PPP. Standard Procedure for the Synthesis of Tether T210

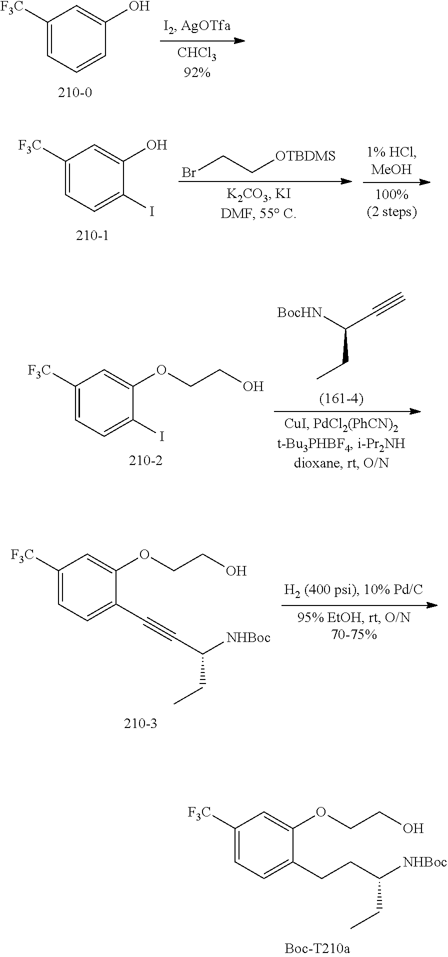

Successive transformations involving iodination of 3-trifluoromethylphenol (210-0), alkylation of the phenol and deprotection of the silyl ether gave intermediate 210-2. Sonogashira coupling with the alkyne 134-3 followed by reduction of the triple bond provided protected tether Boc-T210a. The enantiomeric tether, Boc-T210b, can be synthesized by the same procedure, but using 175-3, the enantiomeric reagent to 161-4.

### QQQ. Standard Procedure for the Synthesis of Tether T211

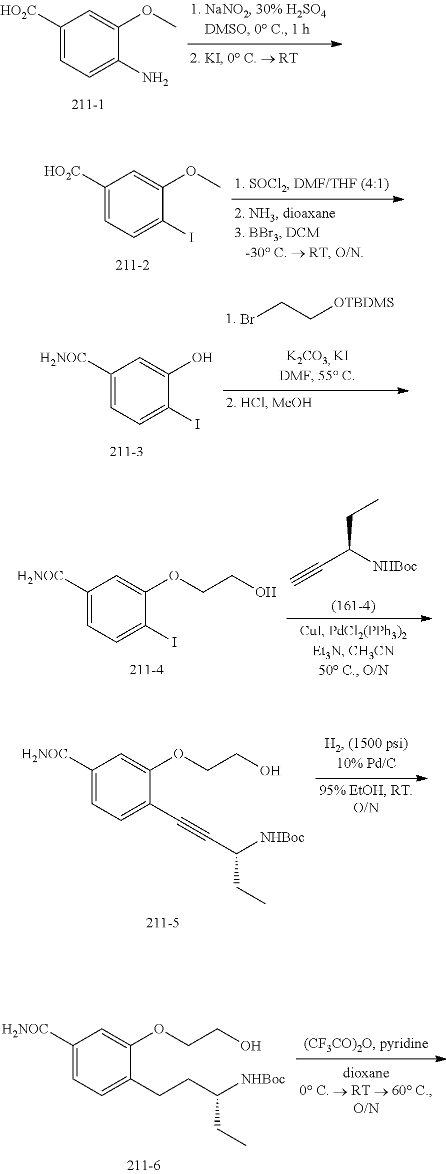

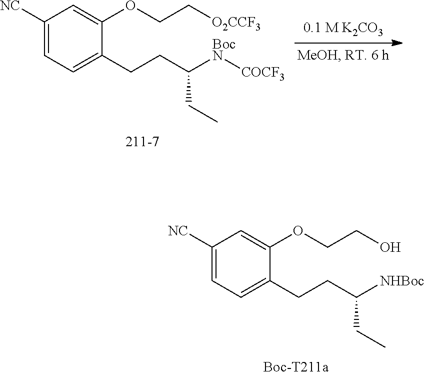

Diazotization of the aniline 211-1 and displacement with iodide gives 211-2. Conversion of the carboxylic acid into the amide under standard methods followed by cleavage of the aromatic methyl ether provides 211-3. Alkylation of the freed phenol and deprotection of the silyl ether is used to prepare the precursor for the Pd(0)-coupling, which is performed in a mariner similar to other such transformations already described. Reduction of the alkyne leads to 211-6, an intermediate which itself could be useful as a tether component. Dehydration of the amide to the nitrile, then removal of the resulting trifluoroacetyl groups yields the target tether, Boc-T211a. The enantiomeric tether, Boc-T211b, can be synthesized by the same procedure, but using 175-3, the enantiomeric reagent to 161-4.

### RRR. Standard Procedure for the Synthesis of Tether T212

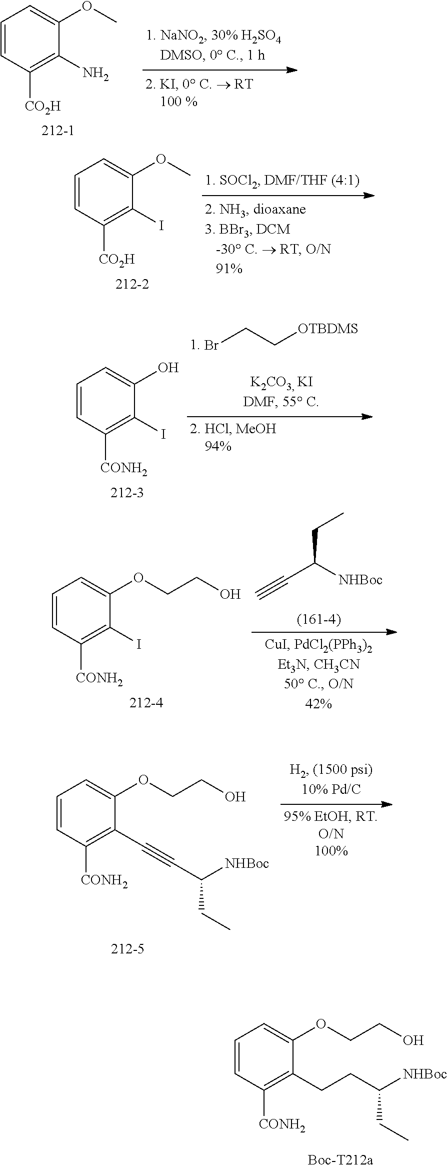

A generally high-yielding sequence starting from the amino acid 212-1 was used to prepare protected tether Boc-T212. Conversion of the amine to the iodide was accomplished through diazotization and treatment with iodide. Transformation of the acid to the amide using the intermediacy of the acyl chloride was followed by boron tribromide cleavage of the methyl ether. Alkylation of the phenol, hydrolytic removal of the silyl protecting group and Sonogashira coupling gave 212-5. Complete reduction of the triple bond then provided Boc-212a. The enantiomeric tether, Boc-T212b, can be synthesized by the same procedure, but using 175-3, the enantiomeric reagent to 161-4.

### SSS. Standard Procedure for the Synthesis of Tether T213

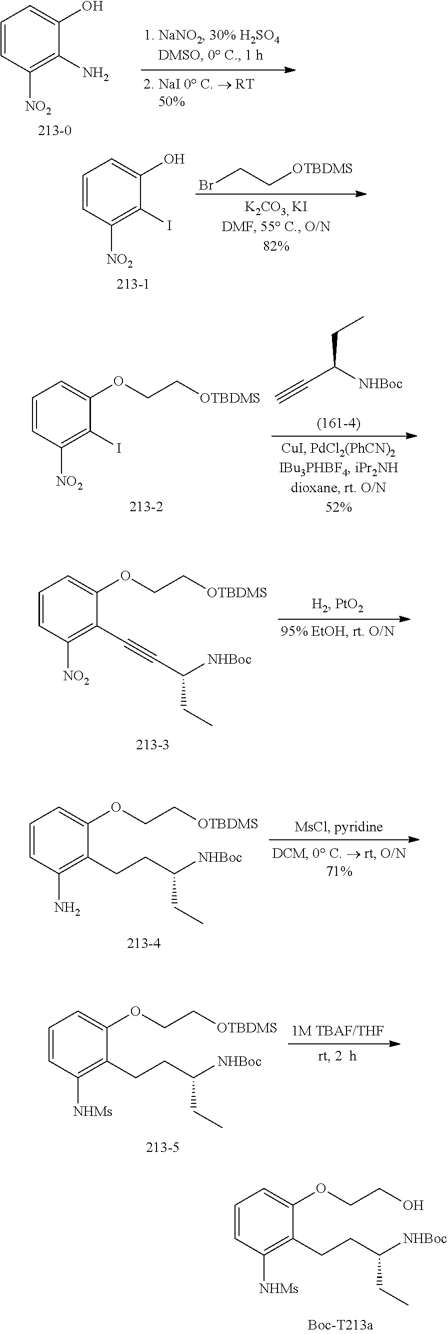

Using the approach described previously, iodide 213-1 was accessed in fair yield from the corresponding aniline, 213-0. Alkylation, Sonogashira reaction and reduction provided 213-4. This intermediate, with orthogonal protection of the aromatic amine could be used as a tether component. In this instance, the amine was converted into the methanesulfonamide under standard conditions. Deprotection of the TBDMS moiety completed the synthesis of Boc-T213a. The enantiomeric tether, Boc-T188b, can be synthesized by the same procedure, but using 175-3, the enantiomeric reagent to 161-4.

### TTT. Standard Procedure for the Synthesis of Tether T214

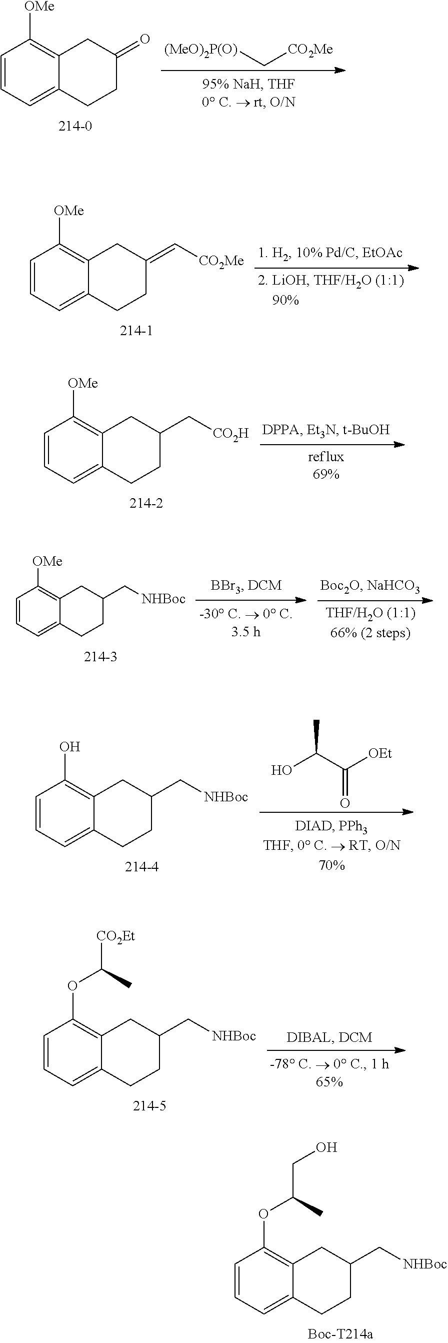

Construction of this tether was initiated by Wittig reaction of the ketone 214-0. The resulting unsaturated product was reduced, then the ester saponified to provide 214-2. Single pot Curtius rearrangement with protection of the amine yielded 214-3. Cleavage of the methyl ether resulted also in loss of the Boc group, therefore requiring reinstallation under standard conditions. (S)-Ethyl lactate was employed in the Mitsunobu reaction of the phenol, which was followed by reduction of the ester to complete the synthesis of Boc-T214a. Use of (R)-ethyl lactate, or other simple ester, in the Mitsunobu for the above procedure accessed the enantiomeric tether Boc-T214b.

### UUU. Standard Procedure for the Synthesis of Tether T215

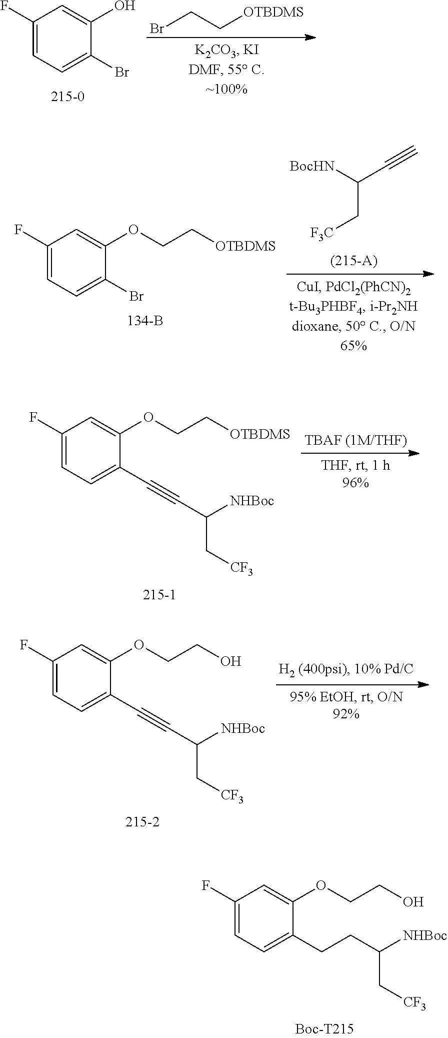

2-Bromo-5-fluorophenol was alkylated utilizing the analogous procedure as already utilized for multiple other tethers. Pd(0)-catalyzed Sonogashira coupling using the racemic alkynyl amine 215-A (synthesized as described below) led in good yields to 215-1. The most efficient process to complete the synthesis was to deprotect the silyl group followed by reduction, which gave Boc-T215.

The key reagent 215-A was prepared from the amino acid 215-0 as illustrated. Reduction of the acid to the alcohol and protection of the amine gave 215-1. Oxidation with Dess-Martin periodinane (DMP) provided the aldehyde, which was converted into the alkyne (215-A) in good yield for the overall process.

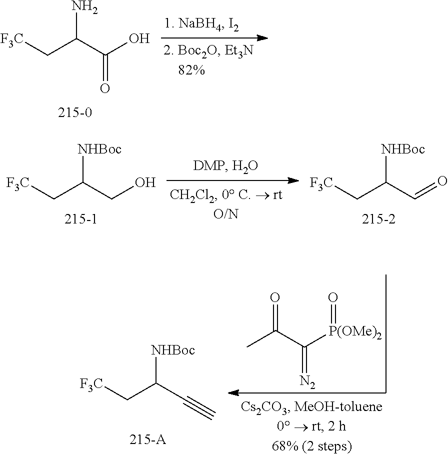

### VVV. Standard Procedure for the Synthesis of Tether T216

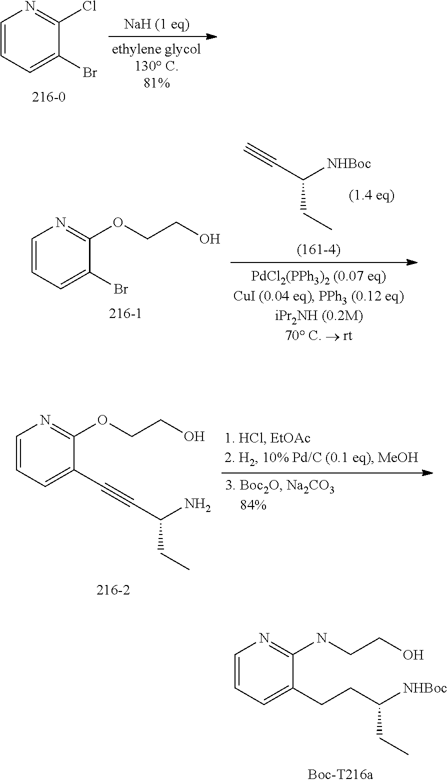

The dihalogenated pyridine 216-0 was subjected to displacement with the anion of ethylene glycol, followed by Sonogashira reaction using 161-4 as the alkyne partner and hydrogenation of the triple bond, to produce Boc-T216a. The enantiomeric tether, Boc-T216b, can be synthesized by the same procedure, but using 175-3, the enantiomeric reagent to 161-4.

### WWW. Standard Procedure for the Synthesis of Tether T217

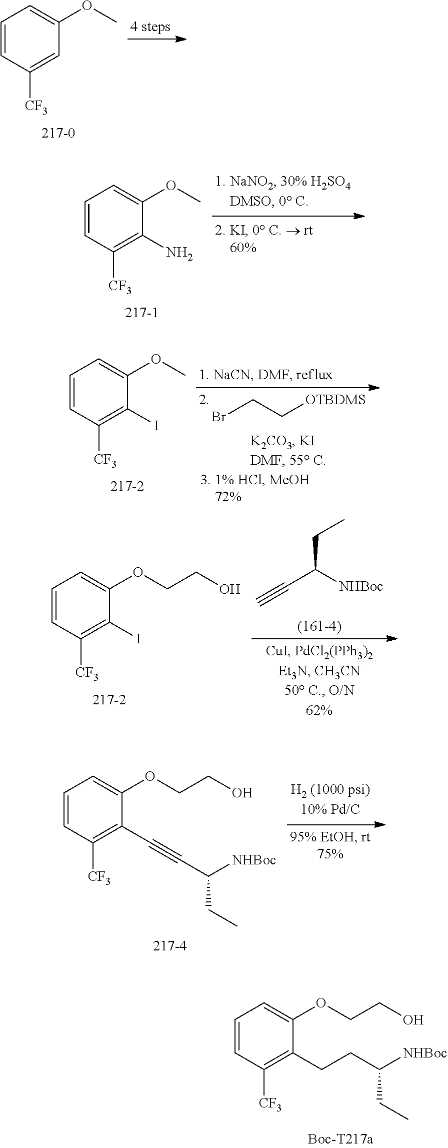

The requisite aniline 217-1 was prepared from 3-trifluoromethylanisole using the procedure described in the literature (Pews, R. G. J. Fluorine Chem. 1998, 87, 65-67). The amine to iodide transformation proceeded via the diazo compound using chemistry as has been described earlier. Nucleophilic removal of the methyl ether with cyanide freed the phenol for subsequent alkylation. Deprotection of the alcohol silyl group provided the coupling precursor 217-3. Following the Sonogashira reaction, reduction of the alkyne gave Boc-T217a. The enantiomeric tether, Boc-T217b, can be synthesized by the same procedure, but using 175-3, the enantiomeric reagent to 161-4.

### XXX. Standard Procedure for the Synthesis of Tether T218

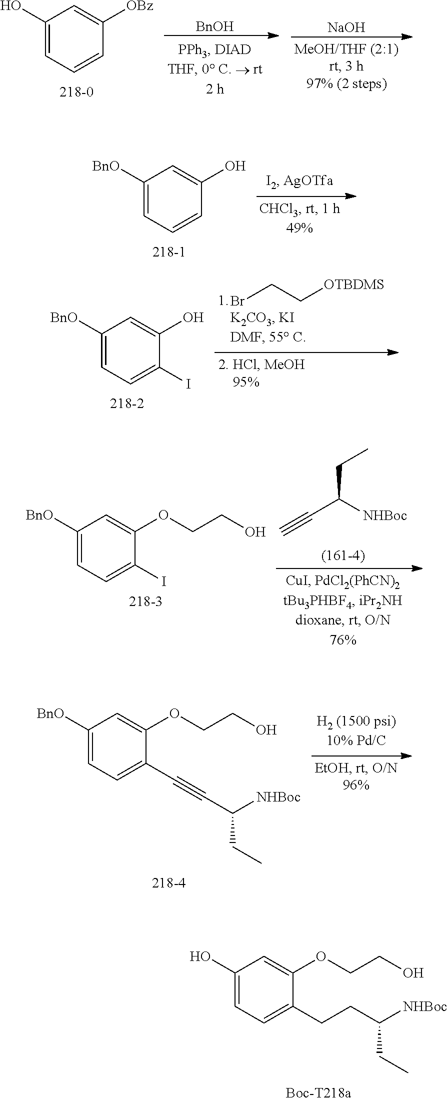

The mono-benzoate of 1,3-dihydroxybenzene, 218-0, was converted into the mono-benzylated derivative, 218-1, in high yield through a protection-deprotection sequence. Iodination in the presence of silver (I) was followed by alkylation and selective silyl ether removal led to 218-3. Coupling with the alkyne 161-4 under Sonogashira conditions was then followed by reduction to provide tether Boc-T218a in very good yield. The enantiomeric tether, Boc-T218b, can be synthesized utilizing the same procedure, but using 175-3, the enantiomeric reagent to 161-4.

### YYY. Standard Procedure for the Synthesis of Tether T219

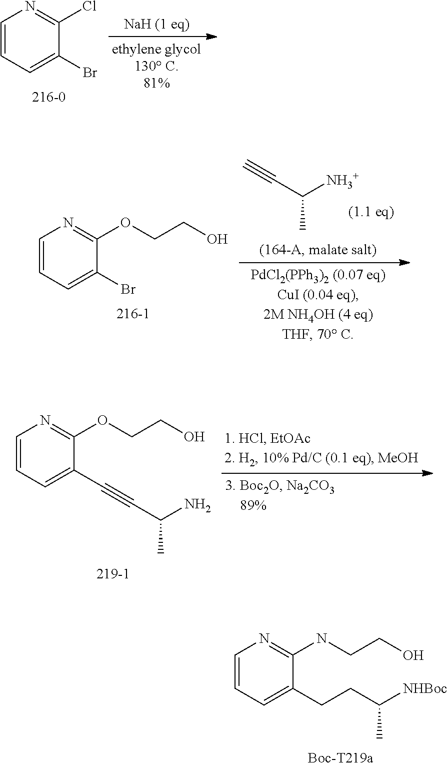

The same intermediate as described previously for T216 was employed to construct this tether as well. Sonogashira reaction of 216-1 with alkyne 164-A provided 219-1. Subsequent reduction of the triple bond and Boc-protection of the amine gave Boc-T219a. The enantiomeric tether, Boc-T219b, can be accessed by the same procedure, but starting from the enantiomeric amino alkyne, 164-B.

### ZZZ. Standard Procedure for the Synthesis of Tether T220

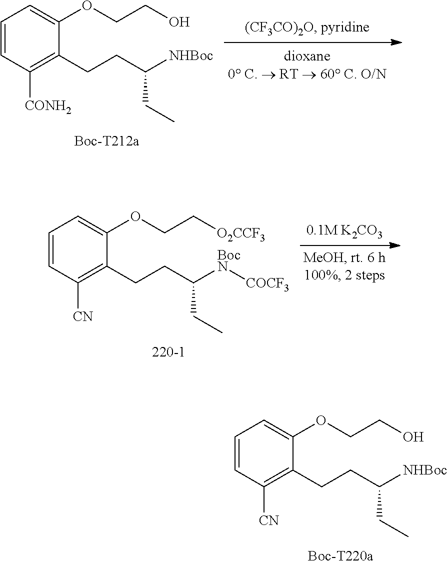

Protected tether T21.2a was utilized in the preparation of this tether as well. Dehydration of the amide to the nitrile by heating with trifluoroacetic anhydride provided 220-1. Removal of the trifluoroacteyl groups on the amine and alcohol with mild basic hydrolysis led to Boc-T220a in essentially quantitative yield. The enantiomeric tether, Boc-T220b, can be synthesized by the same procedure, but using 175-3, the enantiomeric reagent to 161-4, in the preparation of the precursor amide, Boc-T212b.

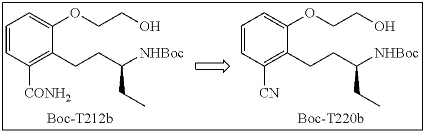

### Example 3

### Macrocyclic Compounds of the Invention

In the construction of macrocyclic compounds of the invention, the amino acids are referred to as AA₁, AA₂ and AA₃ using the same numbering as is standard for peptide sequences, that is from the N- to the C-terminus.

### Example M1

### Standard Procedure for the Synthesis of Compound 1319

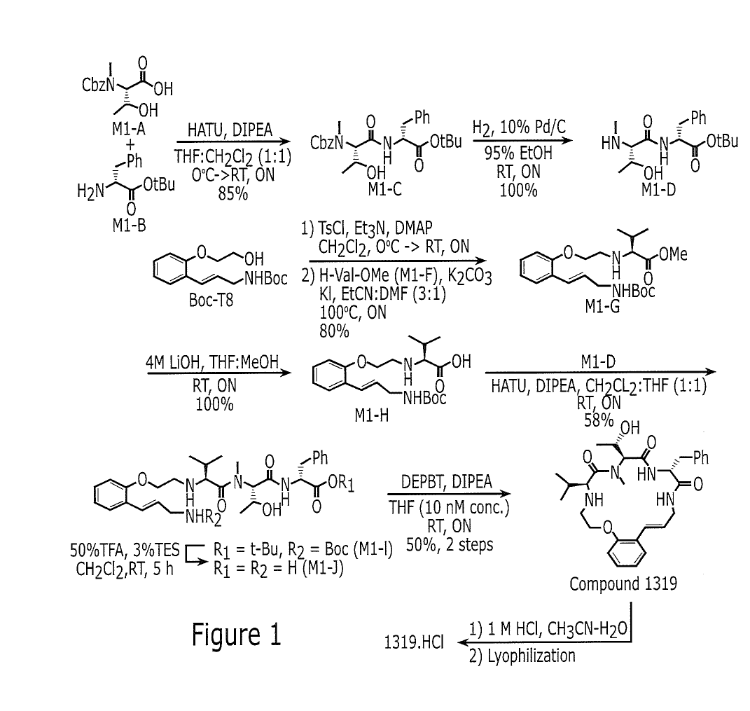

The synthesis of compound 1319 is outlined in FIG. 1.

Step M1-1: Dipeptide formation. To a solution of Cbz-NMeThr-OH (M1-A, 136 mmol, 1.0 eq) in THF/DCM (1:1, 1.15 L) was added H-(D)Phe-OtBu.HCl (M1-B, 150 mmol, 1.1 eq) and HATU (143 mmol, 1.05 eq). The mixture was cooled to 0° C. and DIPEA added. The reaction was stirred at RT for 2-3 d under nitrogen, concluding when HPLC analysis indicated complete disappearance of MI-A. The mixture was then concentrated under reduced pressure to give a yellow oil. This residue was dissolved in DCM and purified by dry pack (50% EtOAc/Hexanes) to give 54 g (85%) of dipeptide MI-C as a yellow solid.

Step M1-2. Cbz deprotection. M1-C (54 g, 115 mmol, 1.0 eq) was dissolved in 95% EtOH (1.6 L) under nitrogen. 10% Pd on C (50% wet) was added and H₂ (g) bubbled into the mixture overnight. The mixture was filtered through a Celite pad and the filtrate concentrated under reduced pressure to provide 38 g (100%) of M1-D as a yellow oil.

Step M1-3. Tosylate formation. To a solution of Boc-T8 (80 g, 0.273 mol, 1.0 eq), triethylamine (76 mL, 0.546 mol, 2.0 eq) and DMAP (6.72 g, 0.055 mol, 0.2 eq) in DCM (359 mL) under nitrogen at 0° C. was added, in 30 mL portions (every 5 min until complete), a solution of tosyl chloride (54.6 g, 0.287 mol, 1.05 eq) in DCM (910 mL). The reaction was stirred overnight at RT with monitoring of the reaction by TLC. A saturated aqueous solution of ammonium chloride was added (1 L) and extracted with DCM (2×600 mL). The organic phases were combined and washed with 0.1 N HCl (3×600 mL) and brine (600 mL). The organic phase was dried with MgSO₄, filtered, and the filtrate concentrated under reduced pressure to provide 116 g of M1-E as an orange oil that was used as obtained in the next step without any further purification.

TLC: R_{f}=0.30 (25% EtOAc/hexanes; detection: IJV, Mo/Ce);

HPLC/MS: Gradient A4, t_{R}=8.22 min, [M]⁺ 447.

Step M1-4. AA1 Alkylation. A solution of M1-E (122 g, 0.273 mol, 1.0 eq) in DMF (139 mL) was degassed under reduced pressure for 30 min. Potassium iodide (dried at 140° C. under vacuum O/N, 113.4 g, 0.683 mol, 2.5 eq), potassium carbonate (113.4 g, 0.819 mol, 3.0 eq), H-Val-OMe (M1-F, 68.7 g, 0.410 mol, 1.5 eq) and propionitrile (EtCN, 417 mL) were then added under a nitrogen atmosphere. The solution was heated at 100° C., O/N with TLC monitoring. Water was added (2.2 L) and the mixture extracted with EtOAc (3×1 L). The organic phases were combined and washed successively with citrate buffer (2×1 L), saturated aqueous solution of sodium bicarbonate (2×1 L) and brine (2×1 L). The organic phase was dried over MgSO₄, filtered, and the filtrate concentrated under reduced pressure to give a yellow oil. This residue was purified by dry pack (gradient, 15% to 25% EtOAc/Hex) to give 87 g (80%) of M1-G as an orange oil.

TLC: R_{f}=0.38 (40% EtOAc/hexanes; detection: UV, Mo/Ce).

Step M1-5. Ester cleavage. To a solution of M1-G (80.0 g, 190 mmol, 1.0 eq) in THF:MeOH (1:1, 1200 mL) was added 4 M LiOH (674 mL) and the mixture agitated (mechanical stirring) overnight. Solvents were evaporated in vacuo to leave a yellow gel. Water was added and the heterogeneous mixture was cooled to 0° C. 3 M HCl was then added to obtain a pH=3-4 and agitation (mechanic stirring) continued. Note that this pH range is important to avoid premature Boc deprotection. A white precipitate formed, which was collected by filtration, rinsed with water, then ether. The precipitate was dissolved in THF and concentrated under reduced pressure. The solid residue was azeotroped with toluene (2×) and THF (1×), then dried under vacuum (oil pump) until ¹H NMR (DMSO-d₆) indicated water remained in only a trace quantity. M1-H (82.2 g, 100%) was thus obtained as a white solid.

Step M1-6. Coupling. To a suspension of MI-H (78.8 g, 184 mmol, 1.5 eq) and M1-D (38.6 g, 115 mmol, 1.0 eq) in THF:CH₂Cl₂ (1:1, 1.5 L) was added HATU (70 g, 184 mmol, 1.5 eq) and DIPEA (120 mL, 690 mmol, 6.0 eq) slowly. Formation of a gel during this addition made the mixture very difficult to stir. The heterogeneous mixture was agitated (mechanical stirring) overnight with TLC monitoring. The solvents were evaporated in vacuo and the residue dissolved in EtOAc. The organic solution was washed successively with citrate buffer (2×), NaHCO₃ sat. aq. (2×) and NaCl sat. aq. (1×). The organic phase was dried over MgSO₄, filtered, then the filtrate concentrated under reduced pressure to leave a yellow oil. This residue was purified by dry pack (30% EtOAc/Hex) to give 68.2 g (58%) of M1-I as a beige foam.

TLC: R_{f}=0.31 (60% EtOAc/hexanes; detection: UV, Mo/Ce),

Step M1-7. Deprotection. M1-I (74.8 g, 105 mmol, 1.0 eq) was stirred in a solution of 50% TFA, 3% TIPS/CH₂Cl₂ (840 mL) 5 h. The solvents were evaporated in mow, toluene added and the mixture again evaporated in vacuo. The residue was dried under vacuum (oil pump) overnight to provide M1-J as a yellow-orange solid that was used without further purification in the next step.

Step M1-8. Macrocycle formation. To a solution of M1-J (105 mmol, 1.0 eq) in THF (10.5 L) were added DEPBT (47.1 g, 158.0 mmol, 1.3 eq) and DIPEA (110 mL, 630.0 mmol, 6.0 eq). The resulting mixture was agitated (mechanical stirring) overnight. The reaction can be monitored by HPLC. Upon completion, THF was evaporated in vacuo and 1 M Na₂CO₃ (aq) added. The aqueous phase was extracted with EtOAc (3×). Then, the combined organic phase was washed with 1 M Na₂CO₃ (aq, lx) and NaCl sat. (aq, lx), dried over MgSO₄, filtered, and the filtrate concentrated under reduced pressure to leave an orange residue. This orange residue was purified by dry Pack (gradient, 3% to 5% MeOH), then the product-containing fractions precipitated in CH₃CN to give compound 1319, 8.2 g (50%, 2 steps).

Step M1-9. HCl salt formation. Approximately 1 g of 1319 was placed in a 40 mL vial and 10 mL of acetonitrile added. To the suspension was added 2 eq of 1 M HCl (3.4 mL) and the resulting mixture diluted with water to obtain 20 mL of total solvent. A concentration of 50 mg/mL of solvents was obtained and the macrocycle was totally soluble. The solvents were frozen in liquid nitrogen for 15 min, then lyophilized for 3 d to obtain the HCl salt of 1319. Using this method, 11.1 g of 1319.HCl was obtained.

### Example M2

### Standard Procedure for the Synthesis of Compound 1346

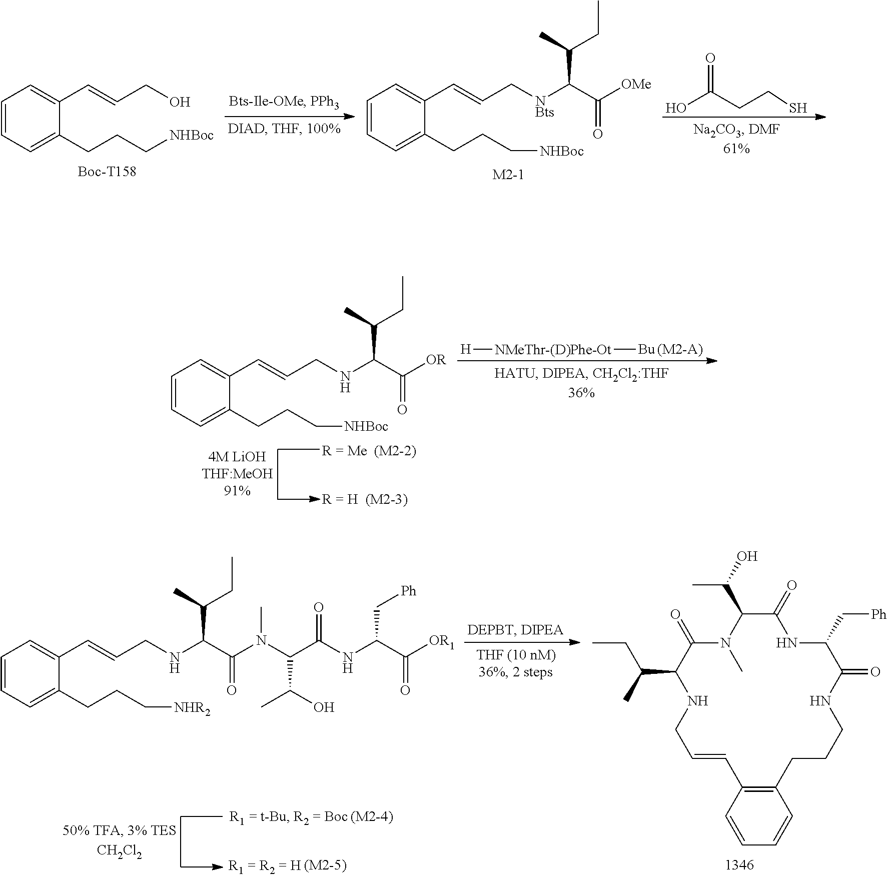

A slightly different, but still convergent, procedure than that used for compound 1319 was employed for the construction of compound 1346. The tether, Boc-T158 was attached to AA₁, Bts-Ile-OMe, using a Mitsunobu reaction to give M2-1. Removal first of the Bts group, which both activated and protected the nitrogen of AA₁, was effected using standard conditions with thiopropionic acid and base, to provide M2-2, then the ester cleaved with lithium hydroxide in THF/MeOH to prepare M2-3. The AA₂-AA₃ dipeptide, H-NMeThr-(D)Phe-Ot-Bu (M2-A), synthesized separately using standard methods, was attached to the AAr-tether component using HATU as coupling agent to afford a low yield of M2-4. The Boc and t-Bu protecting groups were simultaneously removed via the usual method to give the macrocyclization precursor, M2-5. Cyclization with DEPBT under dilute conditions (˜10 nM) gave the product, 1346, in an overall yield of 7.2%, after flash chromatographic purification. In addition, compounds M2-1, M2-2 and M2-4 were purified with flash chromatography, while M2-3 and M2-5 were used crude.

### Example M3

### Standard Procedure for the Synthesis of Compound 1350

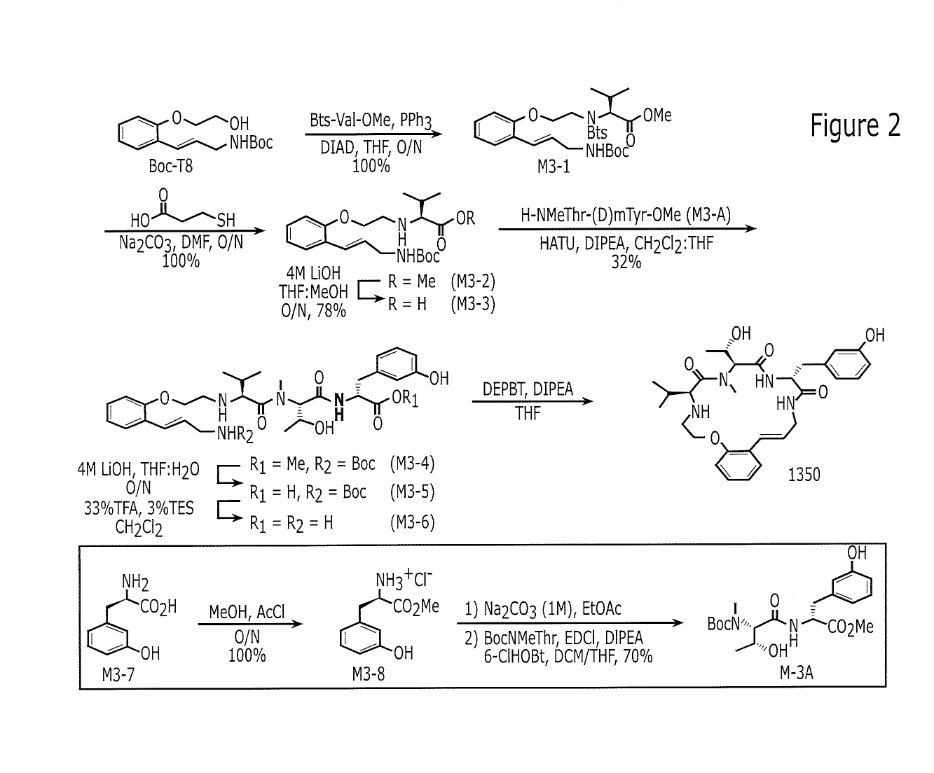

Essentially the same procedure as that used for compound 1346 was employed for the construction of compound 1350 as presented in FIG. 2. The tether, Boc-T8 was attached to AA_{I}, Bts-Val-OMe (1.0 g), using a Mitsunobu reaction to give M3-1 (1.84, 100%). Removal first of the Bts group was performed using standard conditions with thiopropionic acid and base, to provide M3-2 (1.5 g, 100%), then the ester cleaved with lithium hydroxide (or trimethyltin hydroxide) in THF/MeOH to prepare M3-3 (78%). The AA₂-AA₃ dipeptide, H-NMeThr-(D)ₘTyr-OMe (M3-A), was synthesized separately from the protected amino acids M3-7 and M3-8˜in 70% yield on a 2 g scale using standard methods as shown. M3-A was connected to the AA₁-tether component using HATU as coupling agent in DMF (or NMP) to afford a low yield of M3-4. First the methyl ester moiety and then the Boc group were removed via the usual methods to give the macrocyclization precursor, M3-6. Cyclization with DEPBT gave the product, 1350 (6.2 mg) after HPLC purification.

### Example M4

### Standard Procedure for the Synthesis of Compound 1351

The same procedure as that used above for compound 1350 (FIG. 2) was employed for the construction of compound 1351 (30.9 mg), but starting from Bts-Ile-OMe. Coupling to the M3-A dipeptide occurred in 55% yield.

### Example M5

### Standard Procedure for the Synthesis of Compound 1352

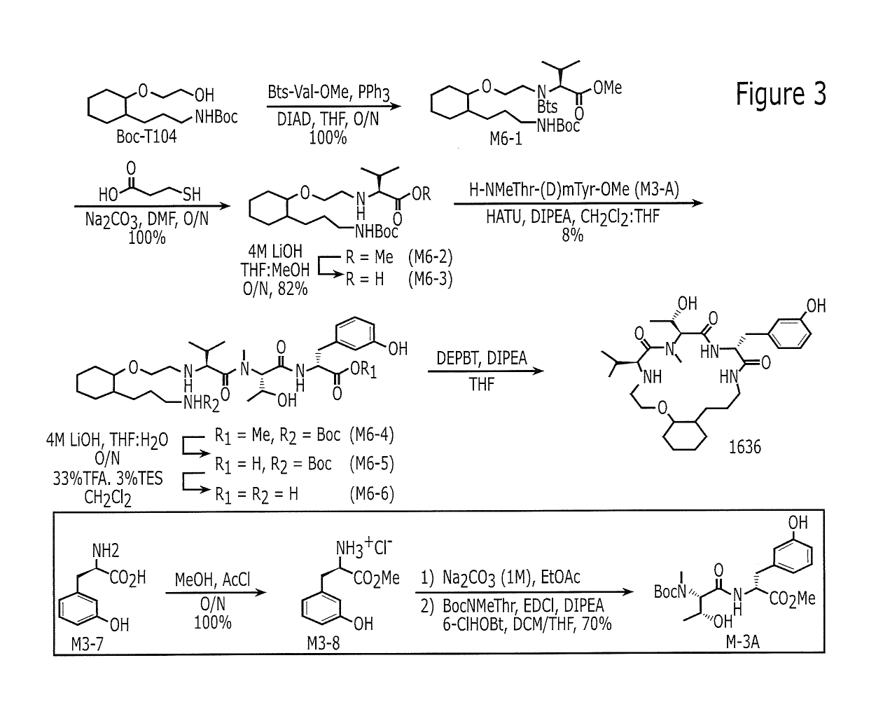

The same procedure as that used above for compound 1350 (FIG. 2) was employed for the construction of compound 1352 (5.0 mg), but starting from the tether T125a. Specific yields obtained through the sequence, starting from 1 g Bts-Val-OMe, were: Ak-tether formation (100%), Bts deprotection (89%), and ester cleavage (100%). Example M6. Standard Procedure for the Synthesis of Compound 1636. As outlined in FIG. 3, the same procedure as that used above for compound 1350 was employed for the construction of compound 1636 (0.2 mg), but starting from the tether T104. In particular, the coupling yield of the AA_{I}-tether component to the dipeptide M3-A was low (8%).

### Example M7

### Standard Procedure for the Synthesis of Compound 1383

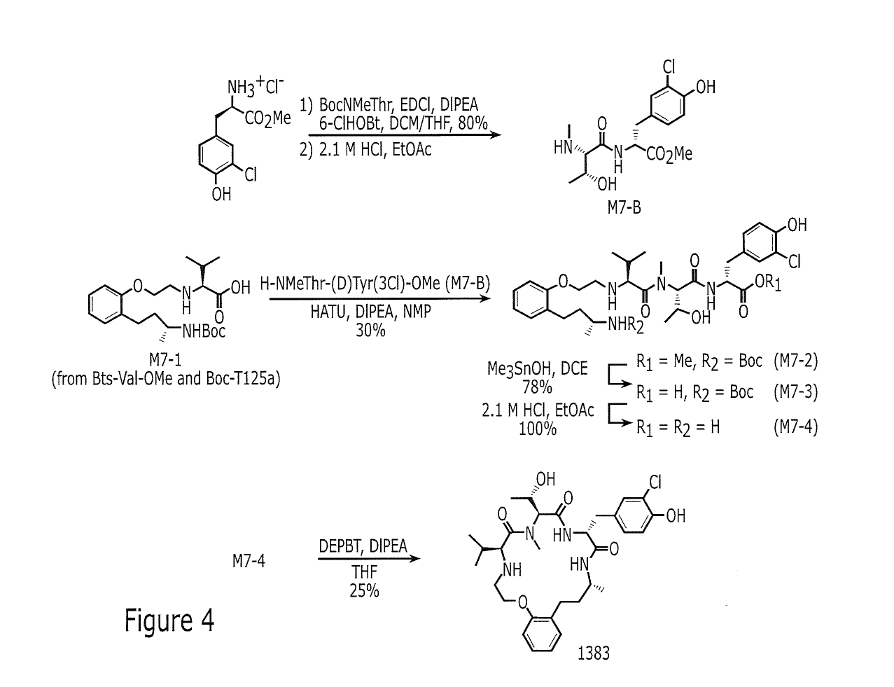

A modified reaction procedure to that already described was employed for the construction of compound 1383 and is provided in FIG. 4. M7-1 was synthesized from Bts-Val-OMe and Boc-T125a as previously described using a Mitsunobu reaction. The AA₂-AA₃ dipeptide, H-NMeThr-(D)Tyr(3-Cl)-OMe (M7-B), was synthesized separately from the protected amino acids Boc-NMeThr-OH and H-(D)Tyr(3-Cl)-OMe (M7-A) as shown in 80% yield after flash chromatography (gradient 80% to 95% EtOAc/Hex). M7-B and M7-1 were connected using HATU as coupling agent in NMP to afford a 30% yield of M7-2 after flash chromatography (gradient 80% to 95% EtOAc/Hex). Next, the methyl ester moiety was cleaved using trimethyltin hydroxide and then the Boc group was removed with HCl in EtOAc to give the macrocyclization precursor, M7-4. Cyclization with DEPBT gave the product, compound 1383 (25% yield, 4.7% overall) after flash chromatography (5% MeOH/EtOAc), then HPLC purification.

### Example M8

### Standard Procedure for the Synthesis of Compound 1390

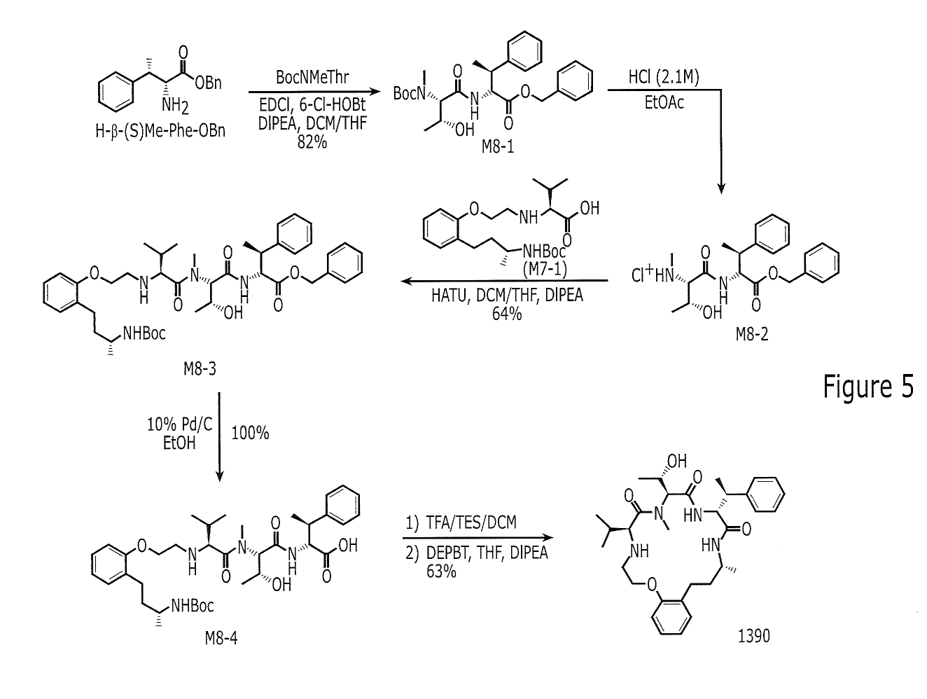

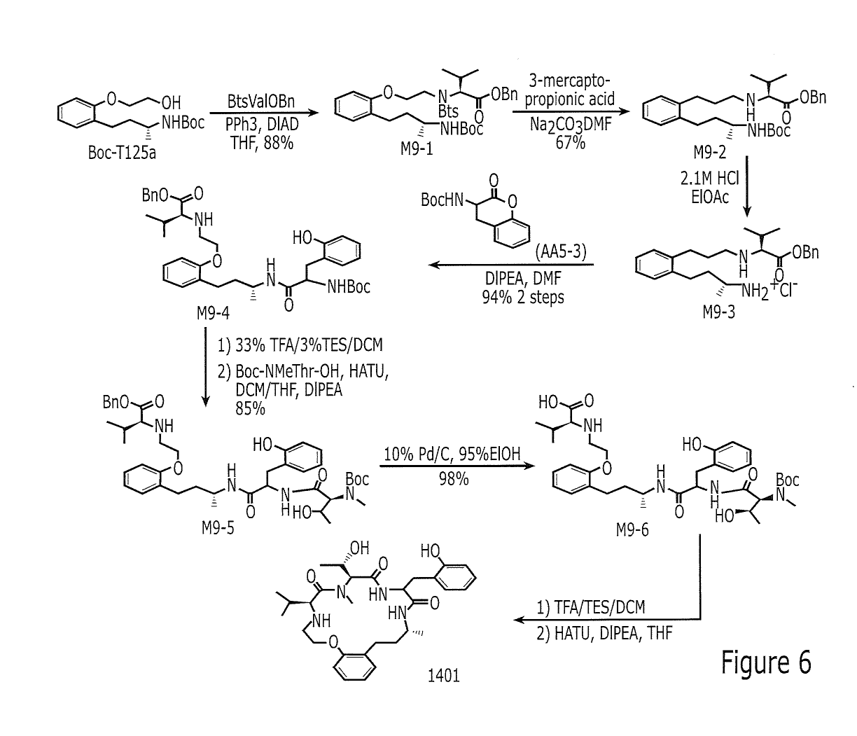

In FIG. 5 is presented the modified reaction procedure to those already described, which was employed for the construction of compound 1390. The dipeptide M8-1 was synthesized from Boc-NMeThr-OH and AA4(Bn) using standard methods. Deprotection of the Boc group with 2.1 M HCl in EtOAc gave M8-2, which was coupled to M7-1 using HATU as coupling agent in DCM/THF to afford a 64% yield of M8-3. Next, the benzyl ester moiety was cleaved using hydrogenolysis, then the Boc group was removed with TFA to give the macrocyclization precursor, M8-4. Cyclization with DEPBT gave the product, compound 1390 (135 mg, 63% yield) after HPLC purification. Example M9. Standard Procedure for the Synthesis of Compound 1401. A different reaction procedure to those already described was employed for the incorporation of the o-Tyr amino acid into the macrocyclic framework as summarized in FIG. 6. M9-1 was synthesized from Bts-Val-OMe and Boc-T125a as previously described using a Mitsunobu reaction. Deprotection of the Bts moiety from this material with 3-mercaptopropionic acid and base provided M9-2, then cleavage of the Boc group with 2.1 M HCl in EtOAc gave M9-3. This was followed by reaction with the Boc-o-Tyr lactone (AA5-3) in the presence of DIPEA as base to afford M9-4. The Boc group of M9-4 was removed and Boc-NMeThr-OH coupled to the resulting deprotected intermediate using HATU to provide M9-5 in 85% yield. Next, the benzyl ester protection was removed by hydrogenolysis to afford M9-6. Deprotection of the Boc group from M9-6, then cyclization with HATU in the presence of DIPEA base gave the product, compound 1401, after HPLC purification.

### Example M10

### Standard Procedure for the Synthesis of Compound 1300

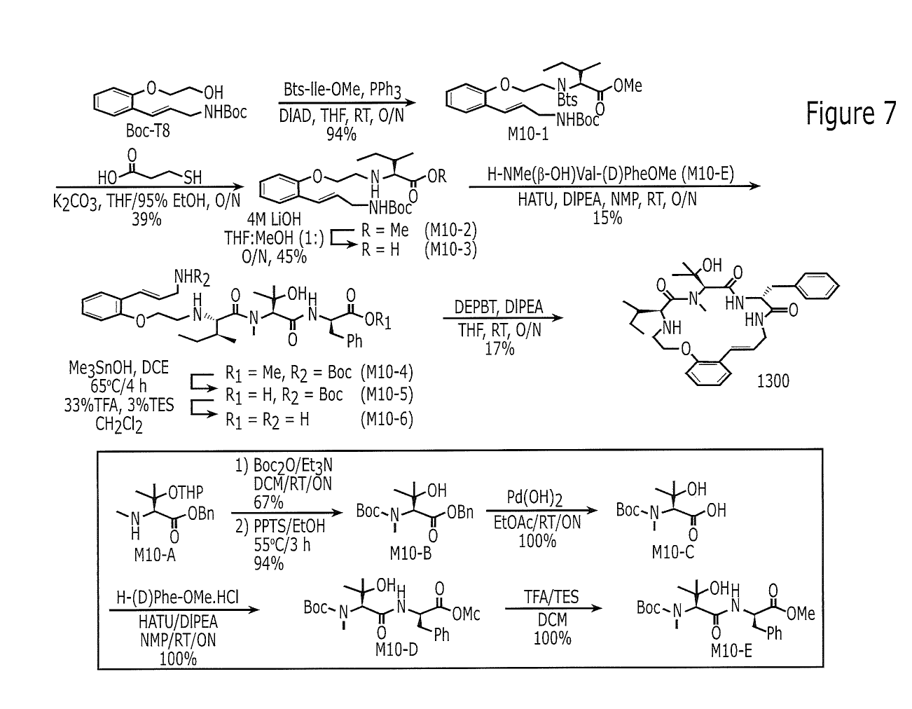

A modified reaction procedure to those already described was employed in order to incorporate the amino acid H-NMe-(β-OH)Val-OH as illustrated for the construction of compound 1300 (see WO 2006/137974) is provided in FIG. 7. M10-1 was synthesized from Bts-Ile-OMe and Boc-T8 as previously described using a Mitsunobu reaction in 94% yield after flash chromatography. Deprotection first of the Bts group, then of the methyl ester, were performed using standard methods to give M10-3. The AA₂-AA₃ dipeptide, H-NMe(β-OH)Val-(D)PheOMe (M10-E), was synthesized separately from the protected amino acids H-NMe(β-OTHP)Val-OBn (M10-A) and H-(D)Phe-OMe. Protecting group modifications to give Boc-NMe(β-OH)Val-OBn (M10-B) in 63% yield after flash chromatography. The benzyl ester protection was removed by hydrogenolysis to provide M10-C, which was connected to H-(D)Phe-OMe.HCl using HATU as coupling agent in NMP to afford a quantitative yield of M10-D after flash chromatography. M10-E was prepared from M10-D by standard cleavage of the Boc group. This derivative, M10-E, in turn, was coupled to M10-3 again using HATU in NMP with D1PEA as base, although in low yield (15%) of M10-4. Next, the methyl ester moiety was cleaved using trimethyltin hydroxide and then the Boc group was removed with TFA/TES to give the macrocyclization precursor, M10-6. Cyclization with DEPBT in dilute conditions (0.01 M) gave the product, compound 1300 (17% yield), after flash chromatographic purification.

### Example M11

### Standard Procedure for the Synthesis of Compound 1505

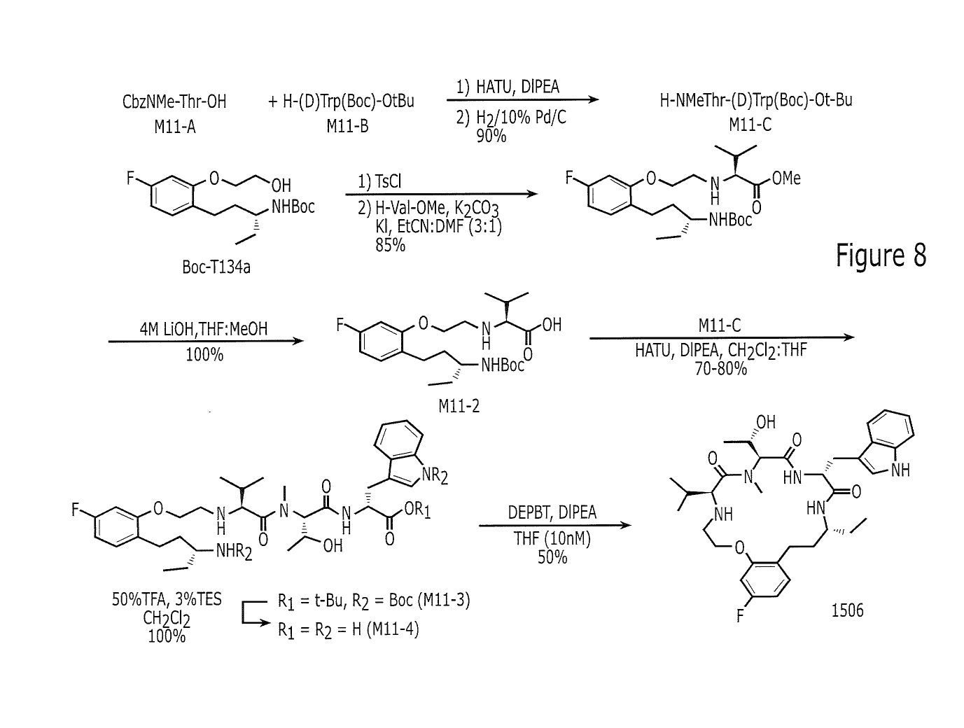

A reaction procedure essentially the same as described in Example M1 was employed to access compound 1505 as outlined in FIG. 8. The dipeptide component, M11-C, was constructed from the protected amino acid derivatives Cbz-NMeThr-OH (M11-A) and H-(D)Trp(Boc)-OtBu (M11-B). M11-A was obtained as its cyclohexylamine (CHA) salt and, therefore, had to be converted to the corresponding free base prior to use as is known to those skilled in the art. As an example, 33 g (140 mmol, 1.0 eq)) of M11-A was prepared from 50 g of the CHA salt. To this was coupled 51 g (140 mmol, 1.0 eq) of M11-B, followed by removal of the Cbz protection under standard hydrogenolysis conditions, to provide 75 g (126 mmol, 90%) of dipeptide M11-C. Separately, tether T134a was converted into the corresponding tosylate then reacted with H-Val-OMe as nucleophile in EtCN-DMF solvent to give M11-1 in 85% yield. Deprotection of the methyl ester with LiOH proceeded in quantitative yield to provide M11-2. This intermediate (105 mmol) was coupled to M11-C (75 g, 126 mmol, 1.2 eq) using HATU to afford M11-3 in 70-80% yield. Simultaneous acidic cleavage of the Boc and tBu protecting groups gave the Macrocyclization precursor M11-4 essentially quantitatively. Cyclization was effected using DEPBT/DIPEA in THF at a dilute concentration of ˜10 nM. The macrocycle 1505 was thus obtained in 50% yield (23 g, 37 mmol) after purification.

### Example 4

### Biological Results

Representative compounds of the invention were evaluated using the methods detailed in Methods B1, for binding activity to the ghrelin receptor, Methods B2 and B3, for functional activity as an antagonist at the ghrelin receptor and Method B4, for functional activity as an inverse agonist at the ghrelin receptor. Results are shown in Tables 7, 8 and 9, respectively.

TABLE 7
Ghrelin Receptor Binding Activity for
Representative Compounds of the Invention

| Compound | Kᵢ (nM) | IC₅₀ (nM) |
|---|---|---|
| 1301 | C | — |
| 1302 | A | B |
| 1304 | B | — |
| 1305 | D | — |
| 1311 | D | — |
| 1313 | B | B |
| 1314 | C | C |
| 1315 | A | A |
| 1316 | B | B |
| 1317 | A | B |
| 1318 | A | B |
| 1319 | A | B |
| 1320 | B | B |
| 1323 | B | — |
| 1324 | B | — |
| 1325 | B | B |
| 1326 | A | B |
| 1327 | A | B |
| 1328 | B | C |
| 1329 | B | C |
| 1330 | B | C |
| 1331 | B | B |
| 1332 | B | C |
| 1333 | B | B |
| 1334 | A | B |
| 1335 | C | D |
| 1336 | B | B |
| 1337 | B | B |
| 1338 | B | B |
| 1339 | C | C |
| 1340 | B | B |
| 1341 | C | D |
| 1342 | A | A |
| 1343 | A | — |
| 1344 | B | — |
| 1345 | B | C |
| 1346 | C | D |
| 1347 | C | C |
| 1348 | C | D |
| 1349 | B | — |
| 1453 | — | A |
| 1503 | | A |
| 1505 | — | A |
| 1535 | B | — |
| 1551 | B | C |
| 1552 | C | C |
| 1554 | D | D |
| 1555 | C | — |
| 1556 | B | C |
| 1558 | C | C |
| 1559 | C | C |
| 1560 | C | D |
| 1601 | A | — |
| 1655 | A | A |
| 1688 | — | A |
| 1689 | — | B |
| 1690 | — | A |
| 1691 | — | A |
| 1692 | — | A |
| 1693 | — | B |
| 1694 | — | D |
| 1695 | — | C |
| 1696 | — | D |
| 1697 | — | C |
| 1698 | — | B |
| 1699 | — | B |
| 1700 | — | A |
| 1701 | — | A |
| 1702 | — | A |
| 1703 | — | A |
| 1704 | — | B |
| 1705 | — | B |
| 1706 | — | C |
| 1707 | — | B |
| 1708 | — | C |
| 1709 | — | B |
| 1710 | — | B |
| 1711 | — | A |
| 1712 | — | A |
| 1713 | — | A |
| 1714 | — | A |
| 1715 | — | A |
| 1718 | — | A |
| 1719 | — | B |
| 1720 | — | B |
| 1721 | — | C |
| 1722 | — | B |
| 1723 | — | B |
| 1724 | — | B |
| 1725 | — | B |
| 1726 | — | B |
| 1727 | — | D |
| 1728 | — | B |
| 1729 | — | A |
| 1730 | — | A |
| 1731 | — | C |
| 1732 | — | B |
| 1733 | — | C |
| 1735 | — | B |
| 1736 | — | B |
| 1737 | — | A |
| 1738 | — | A |
| 1739 | — | A |
| 1740 | — | A |
| 1741 | — | D |
| 1742 | — | B |
| 1743 | — | B |
| 1744 | — | D |
| 1745 | — | B |
| 1746 | — | A |
| 1747 | — | B |
| 1751 | — | A |
| 1752 | — | B |
| 1753 | — | B |
| 1754 | — | A |
| 1755 | — | A |
| 1756 | — | B |
| 1757 | — | B |
| 1758 | — | A |
| 1759 | — | A |
| 1760 | — | A |
| 1761 | — | B |
| 1762 | — | B |
| 1763 | — | A |
| 1764 | — | D |
| 1768 | — | B |
| 1769 | — | B |
| 1770 | — | C |
| 1771 | — | A |
| 1772 | — | A |
| 1773 | — | B |
| 1774 | — | B |
| 1775 | — | A |
| 1776 | — | A |
| 1777 | — | A |
| 1778 | — | B |
| 1779 | — | B |
| 1780 | — | B |
| 1781 | — | D |
| 1782 | — | D |
| 1784 | — | C |
| 1785 | — | C |
| 1786 | — | C |
| 1787 | — | C |
| 1789 | — | A |
| 1790a | — | A |
| 1790b | — | C |
| 1791 | — | A |
| 1792a | — | A |
| 1792b | — | C |
| 1794 | — | A |
| 1795 | — | A |
| 1796 | — | A |
| 1797 | — | B |
| 1798 | — | A |
| 1799 | — | A |
| 1800 | — | B |
| 1801 | — | A |
| 1802 | — | A |
| 1803 | — | A |
| 1805 | — | B |
| 1806 | — | B |
| 1808 | — | A |
| 1809 | — | A |
| 1810 | — | A |
| 1811 | — | B |
| 1812 | — | B |
| 1813 | — | C |
| 1814 | — | C |
| 1815 | — | A |
| 1824 | — | B |
| 1825 | — | A |
| 1826 | — | C |
| 1827 | — | B |
| 1840 | — | D |
| 1841 | — | D |
| 1842 | — | C |
| 1843 | — | B |
| 1843 | — | B |
| 1844 | — | B |
| 1846 | — | C |
| 1847 | — | C |
| 1848a | — | A |
| 1848b | — | B |
| 1849 | — | B |
| 1851 | — | D |
| 1852 | — | D |
| 1853 | — | B |
| 1854 | — | C |
| 1855 | — | B |
| 1856 | — | B |
| 1857 | — | D |
| 1858a | — | A |
| 1858b | — | B |
| 1859 | — | B |
| 1860a | — | A |
| 1860b | — | B |
| 1861a | — | B |
| 1861b | — | C |
| 1862 | — | D |
| 1863 | — | D |
| 1864 | — | D |
| 1866 | — | D |
| 1867 | — | B |
| 1869 | — | B |
| 1870 | — | B |
| 1871 | — | B |
| 1872 | — | A |
| 1875 | — | A |
| 1876 | — | A |
| 1878 | — | A |
| 1879 | — | B |
| 1880 | — | A |
| 1883 | — | B |
| 1884 | — | A |
| 1885 | — | C |
| 1888 | — | D |
| 1889 | — | C |
| 1891 | — | C |
| 1892 | — | D |
| 1893 | — | D |
| 1894 | — | D |
| 1895 | — | C |
| 1896 | — | C |
| 1897 | — | C |
| 1898 | — | C |
| 1899 | — | B |
| 1900a | — | B |
| 1900b | — | D |
| 1901 | — | C |
| 1902a | — | B |
| 1902b | — | B |
| 1903a | — | B |
| 1903b | — | C |
| 1903c | — | C |
| 1904 | — | A |
| 1905a | — | C |
| 1905b | — | C |
| 1906 | — | B |
| 1907 | — | B |
| 1912 | — | D |
| 1913 | — | B |
| 1916 | — | A |
| 1918 | — | A |
| 1919 | — | A |
| 1921 | — | C |
| 1922a | — | A |
| 1922b | — | B |
| 1925 | — | D |
| 1927 | — | A |
| 1928 | — | B |
| 1929 | — | A |

*Activity, both Kᵢ and IC₅₀, expressed as follows: A = 1-1-0 nM, B = 10-100 nM, C = 100-500 nM; D > 500 nM

TABLE 8
Antagonist Activity of Representative
Compounds of the Invention

| | Antagonist |
|---|---|
| Compound | Activity |
| 1302 | C |
| 1304 | C |
| 1315 | C |
| 1316 | D |
| 1317 | C |
| 1318 | C |
| 1324 | D |
| 1325 | C |
| 1326 | C |
| 1332 | D |
| 1334 | C |
| 1343 | C |
| 1350 | C |
| 1351 | C |
| 1352 | C |
| 1358 | C |
| 1361 | B |
| 1363 | C |
| 1364 | B |
| 1366 | B |
| 1370 | C |
| 1371 | B |
| 1372 | A |
| 1373 | A |
| 1374 | B |
| 1375 | B |
| 1376 | C |
| 1378 | C |
| 1380 | B |
| 1381 | B |
| 1383 | B |
| 1384 | C |
| 1387 | B |
| 1390 | C |
| 1391 | A |
| 1392 | A |
| 1393 | B |
| 1394 | D |
| 1396 | C |
| 1399 | B |
| 1400 | A |
| 1401 | B |
| 1402 | B |
| 1404 | B |
| 1411 | B |
| 1413 | B |
| 1416 | A |
| 1418 | B |
| 1432 | B |
| 1436 | B |
| 1442 | C |
| 1446 | B |
| 1451 | B |
| 1453 | B |
| 1455 | B |
| 1458 | B |
| 1460 | B |
| 1464 | B |
| 1479 | B |
| 1482 | B |
| 1486 | B |
| 1490 | B |
| 1503 | B |
| 1504 | B |
| 1505 | B |
| 1512 | B |
| 1515 | B |
| 1518 | B |
| 1521 | B |
| 1526 | B |
| 1529 | B |
| 1531 | B |
| 1532 | B |
| 1601 | C |
| 1602 | C |
| 1604 | C |
| 1619 | C |
| 1625 | C |
| 1630 | B |
| 1633 | C |
| 1635 | B |
| 1655 | C |
| 1688 | A |
| 1692 | C |
| 1693 | C |
| 1699 | C |
| 1703 | B |
| 1705 | C |
| 1707 | B |
| 1713 | B |
| 1718 | B |
| 1719 | C |
| 1720 | B |
| 1726 | B |
| 1729 | B |
| 1739 | B |
| 1740 | C |
| 1746 | B |
| 1747 | B |
| 1751 | B |
| 1752 | C |
| 1753 | B |
| 1754 | B |
| 1755 | B |
| 1763 | B |
| 1773 | B |
| 1774 | B |
| 1775 | C |
| 1776 | B |
| 1777 | B |
| 1778 | B |
| 1780 | B |
| 1789 | B |
| 1790a | C |
| 1799 | C |
| 1801 | B |
| 1803 | B |
| 1804 | B |
| 1805 | C |
| 1806 | C |
| 1808 | B |
| 1809 | B |
| 1810 | B |
| 1812 | C |
| 1843 | A |
| 1848 | A |
| 1876 | A |
| 1878 | A |
| 1903 | A |
| 1918 | B |
| 1929 | B |

*Activity is expressed as follows: A <1 nM; B = 1-10 nM, C = 10-100 nM, D = 100-500 nM

TABLE 9
Inverse Agonist Activity of Representative
Compounds of the Invention

| Compound | IC₅₀ |
|---|---|
| 1338 | D |
| 1408 | B |
| 1453 | B |
| 1503 | B |
| 1505 | D |
| 1688 | C |
| 1690 | C |
| 1691 | D |
| 1692 | D |
| 1693 | D |
| 1699 | D |
| 1700 | C |
| 1701 | B |
| 1702 | C |
| 1703 | C |
| 1704 | D |
| 1705 | D |
| 1707 | D |
| 1710 | D |
| 1711 | B |
| 1712 | C |
| 1713 | C |
| 1718 | C |
| 1719 | D |
| 1720 | D |
| 1723 | D |
| 1725 | D |
| 1726 | C |
| 1729 | C |
| 1730 | D |
| 1732 | D |
| 1737 | C |
| 1738 | B |
| 1739 | D |
| 1740 | D |
| 1742 | B |
| 1743 | D |
| 1745 | D |
| 1746 | C |
| 1747 | C |
| 1751 | D |
| 1752 | D |
| 1753 | C |
| 1754 | C |
| 1755 | C |
| 1758 | B |
| 1759 | C |
| 1760 | C |
| 1761 | C |
| 1762 | D |
| 1763 | D |
| 1768 | B |
| 1769 | D |
| 1771 | D |
| 1772 | D |
| 1773 | D |
| 1774 | C |
| 1775 | D |
| 1776 | C |
| 1777 | C |
| 1778 | D |
| 1780 | C |
| 1789 | D |
| 1790a | D |
| 1791 | C |
| 1792a | C |
| 1794 | B |
| 1795 | D |
| 1796 | B |
| 1797 | D |
| 1798 | D |
| 1799 | C |
| 1801 | B |
| 1802 | B |
| 1803 | B |
| 1804 | B |
| 1805 | D |
| 1806 | D |
| 1808 | B |
| 1809 | B |
| 1810 | B |
| 1811 | D |
| 1812 | C |
| 1813 | D |
| 1814 | D |
| 1815 | D |
| 1824 | D |
| 1825 | C |
| 1827 | D |
| 1843 | B |
| 1847 | B |
| 1848a | B |
| 1848b | D |
| 1853 | D |
| 1854 | D |
| 1855 | D |
| 1858a | C |
| 1858b | D |
| 1859 | C |
| 1860a | C |
| 1862 | D |
| 1863 | D |
| 1867 | D |
| 1872 | B |
| 1875 | C |
| 1876 | C |
| 1878 | B |
| 1879 | B |
| 1884 | B |
| 1903a | B |
| 1904 | B |
| 1916 | B |
| 1918 | C |
| 1919 | B |
| 1922a | C |
| 1927 | C |
| 1928 | C |
| 1929 | C |

*Activity is expressed as follows: A = 1-10 nM; B = 10-50 nM, C: 50-100 nM, D: 100-500 nM

### Example 5

A detailed analysis of the pharmacokinetic profile of representative compounds of the invention was conducted using the procedures outlined in Method B9. Results for both intravenous and oral administration are provided in Tables 10a and 10b.

TABLE 10a
Pharmacokinetic Parameters for Representative Compounds of the
Invention

| | Compound | Compound | Compound | Compound |
|---|---|---|---|---|
| Compound | 1777 | 1848 | 1929 | 1712 |

Intravenous

| | | | | | |
|---|---|---|---|---|---|
| Dose | mg/kg | 2 | 2 | 2 | 2 |
| t_{1/2} | min | 107 ± 4 | 108 ± 51 | 170 ± 62 | 138 ± 86 |
| Cl | mL/min/kg | 6 ± 2 | 17 ± 9 | 32 ± 4 | 62 ± 10 |
| Vz | mL/kg | 882 ± 272 | 2554 ± 1467 | 7992 ± 3702 | 13237 ± 9572 |
| AUC_{inf} | ng · min/mL | 369904 ± 127112 | 155874 ± 115129 | 63809 ± 8606 | 32618 ± 5193 |

Oral

| | | | | | |
|---|---|---|---|---|---|
| Dose | mg/kg | 8 | 8 | 8 | 8 |
| Cₘₐₓ | ng/mL | 1075 ± 772 | 421 ± 16 | 628 ± 766 | 352 ± 297 |
| Tₘₐₓ | min | 15/30/15/15/15/15 | 30/30 | 15/15/30 | 15/15/15 |
| AUC_{inf} | ng · min/mL | 190433 ± 114760 | 66708 ± 12061 | 107429 ± 130596 | 20174 ± 12692 |
| F | % | 13 ± 8 | 11 ± 2 | 42 ± 51 | 15 ± 10 |

Pharmacokinetic data on additional representative compounds of the invention are provided in Table 10b. A dose level of 2 mg/mL for intravenous administration and 8 mg/mL for oral administration were typically employed.

TABLE 10b
Pharmacokinetic Data for Representative Compounds of the Invention

| Compound | t_{1/2} (min) | Cl (mL/min/kg) | % F |
|---|---|---|---|
| 1693 | 41 ± 4 | 35 ± 18 | 5 ± 2 |
| 1703 | 147 ± 52 | 9 ± 6 | 12 ± 6 |
| 1705 | 166 ± 2 | 6 ± 2 | 50 ± 14 |
| 1707 | 130 ± 26 | 8 ± 4 | nd |
| 1713 | 104 ± 25 | 30 ± 1 | 14 ± 9 |
| 1718 | 162 ± 4 | 5 ± 1 | 17 ± 6 |
| 1719 | 93 ± 11 | 44 ± 3 | nd |
| 1720 | 70 ± 15 | 33 ± 12 | nd |
| 1726 | 171 ± 16 | 9 ± 6 | 14 ± 8 |
| 1746 | 107 ± 4 | 12 ± 1 | 59 ± 31 |
| 1751 | 46 ± 1 | 32 ± 5 | 34 ± 19 |
| 1754 | 106 ± 13 | 8 ± 1 | 39 ± 44 |
| 1755 | 123 ± 28 | 12 ± 13 | 7 ± 4 |
| 1759 | 119 ± 101 | 14 ± 2 | nd |
| 1773 | 70 ± 8 | 24 ± 11 | 2 ± 1 |
| 1775 | 74 ± 27 | 18 ± 16 | 36 ± 29 |
| 1776 | 105 ± 20 | 8 ± 4 | nd |
| 1778 | 59 ± 38 | 26 ± 18 | nd |
| 1789 | 52 ± 1 | 26 ± 8 | 81 ± 55 |
| 1803 | 103 ± 13 | 10 ± 1 | nd |
| 1847 | 70 ± 42 | 19 ± 16 | 10 ± 1 |
| 1876 | 159 ± 16 | 28 ± 8 | 54 ± 19 |
| 1878 | 124 ± 19 | 35 ± 1 | nd |
| 1903a | 31 ± 13 | 17 ± 9 | nd |
| 1904 | 65 ± 25 | 34 ± 4 | nd |
| 1918 | 114 ± 53 | 14 ± 7 | nd |

nd = not determined

### Example 6

### In Vivo Evaluation in Animal Models of Metabolic Disease

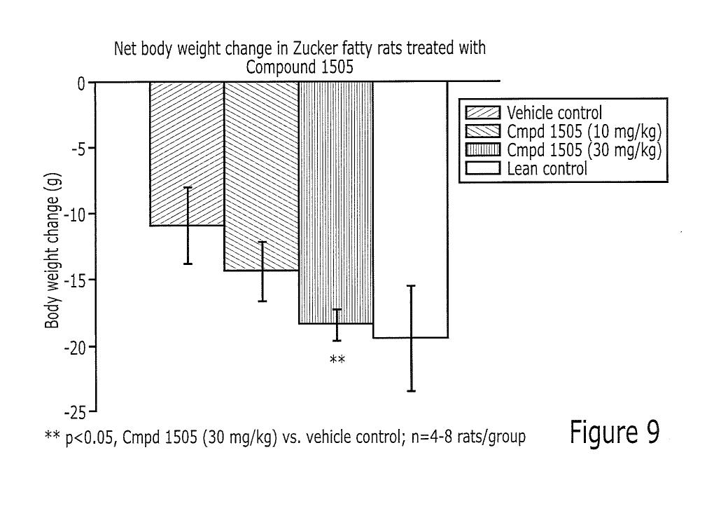

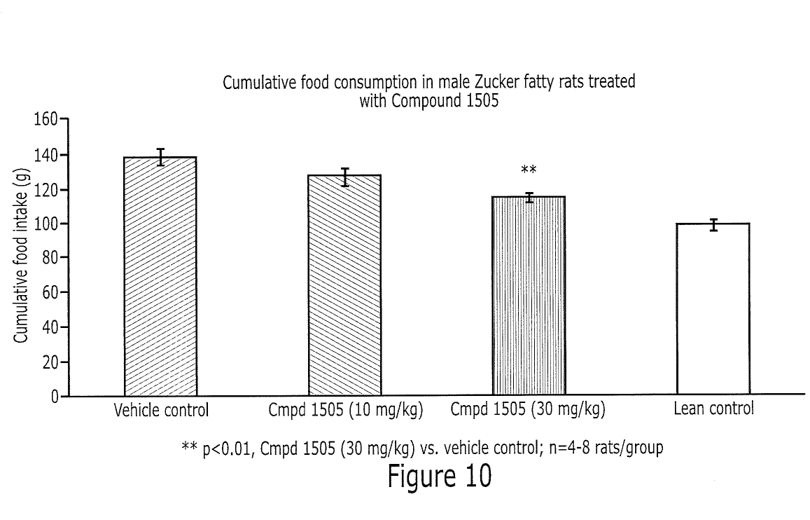

A study of the effects of compound 1505 on metabolic parameters in the Zucker fatty rat, a standard model for the study of anti-obesity or anti-diabetes treatments, using Method B14 was performed. As shown in FIG. 9, this compound at 30 mg/kg demonstrated significant reduction in net body weight over the course of the 7 day study period. Additionally, at this dose level, a significant decrease in the cumulative food consumption was also observed (FIG. 10). On a daily basis, both the 10 mg/kg and 30 mg/kg doses exhibited significant reductions when compared to vehicle controls at the 2 clay timepoint. The higher dose remained significant through the 6 day timepoint.

In addition to the effect on weight, the OGTT results with compound 1505 (30 mg/kg) showed a decrease in blood glucose versus untreated controls at both day 3 and day 7. A lowering effect on insulin levels, as indicated by the area under the curve (AUC), was also obtained in this test. The insulin sensitivity index was higher, attaining significance at the higher dose.

Lastly, other metabolic parameters, including free fatty acids and total cholesterol, were also significantly reduced in both treatment groups. PK analysis demonstrated that sufficient plasma levels of compound 1505 were achieved confirming the efficacy of the molecule upon oral administration.

### Example 7

### In vivo Evaluation in a Further Animal Model of Metabolic Disease

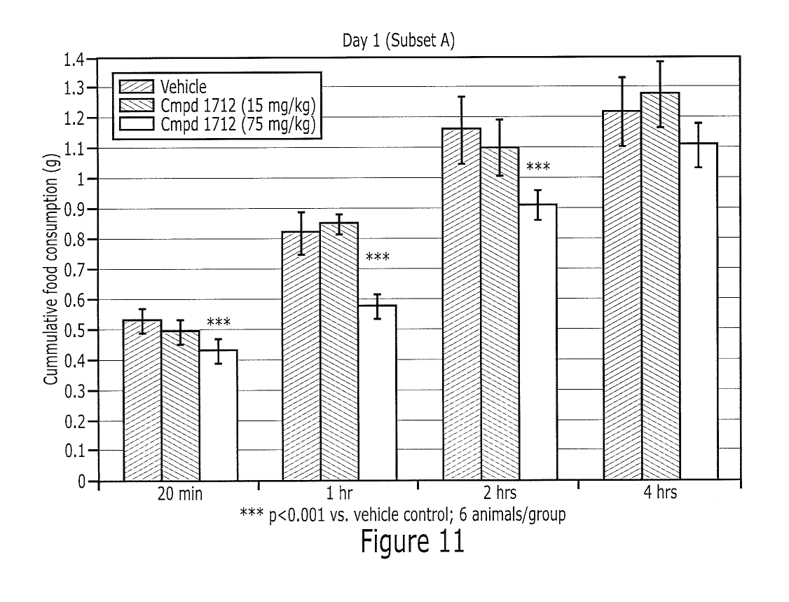

A study of the effects of compounds 1712 and 1848 on metabolic parameters in the ob/ob mouse, a standard model for the study of treatment of metabolic disorders, was conducted using Method B15. As expected in the ob/ob mouse model, the animals were obese and showed aspects of the metabolic syndrome (e.g. hyperinuslinemia, glucose intolerance, dyslipidemia). (Leiter, E. H. FASEB J. 1989, 3, 2231-2241.) As shown in FIG. 11, acute cumulative food intake over a 2 hr period, in fasted animals, was significantly reduced by treatment with compound 1712 compared to vehicle control animals.

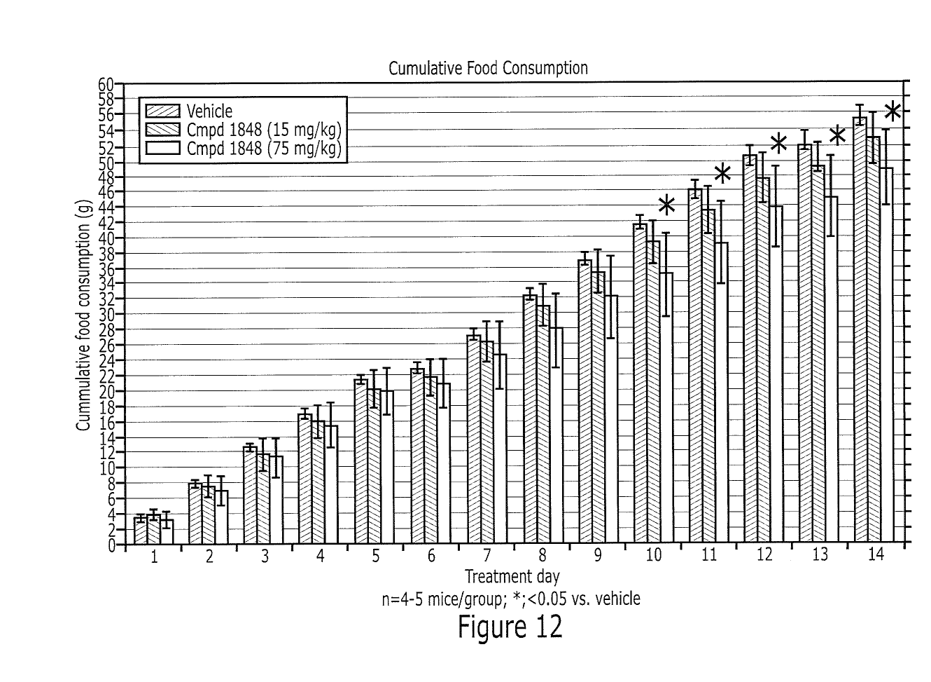

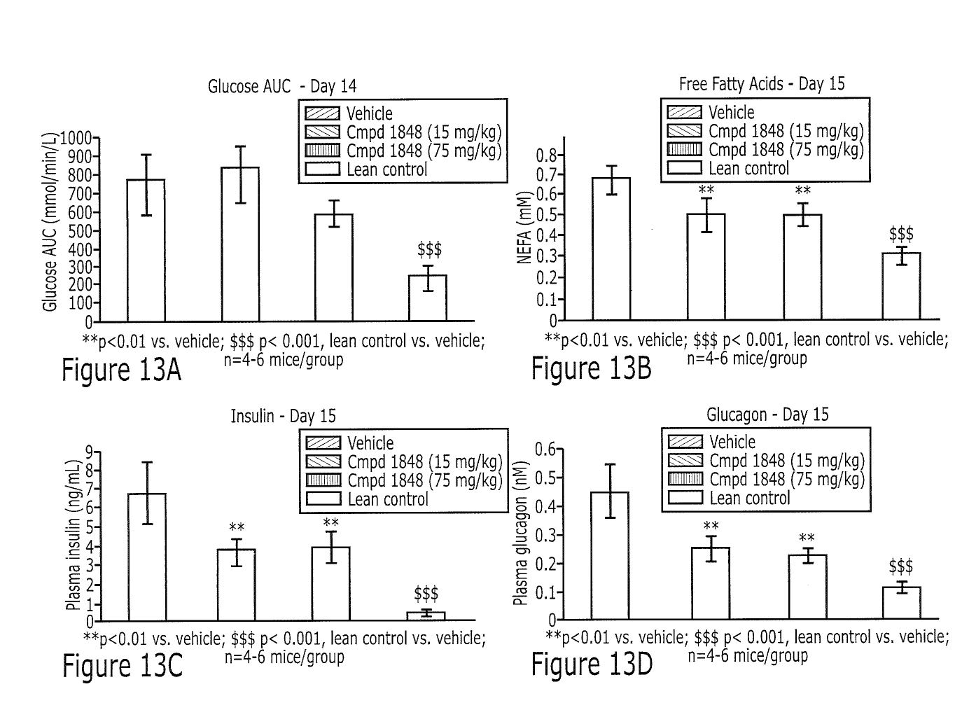

In a separate 14 d study, a significant reduction in cumulative food intake (11.9%) at a dose of 75 mg/kg was observed for the compound 1848 treated animals compared to the vehicle control (FIG. 12). In addition, a significant decrease was seen in blood glucose levels during an oral glucose tolerance test in the compound 1848 (75 mg/kg) treated mice compared to vehicle control suggesting improvement in glucose tolerance upon treatment. On other metabolic parameters, treatment with compound 1848 significantly reduced non-fasting glucose, insulin, glucagon, free fatty acids (FFAs), but not total cholesterol or triglycerides levels compared to vehicle control mice (FIG. 13). These data indicate an improvement in insulin sensitivity in compound 1848-treated ob/ob mice.

The foregoing is illustrative of the present invention and is not to be construed as limiting thereof. The invention is defined by the following claims, with equivalents of the claims to be included therein.

What is claimed is:

1. A compound of the formula (I): 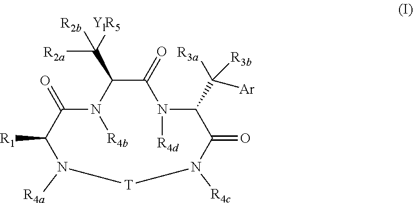
or a pharmaceutically acceptable salt thereof, wherein:
T is selected from 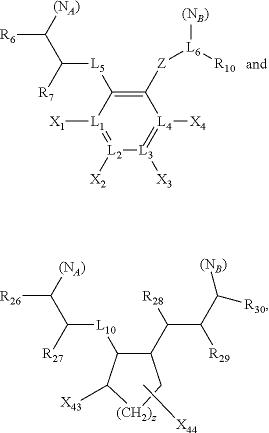
wherein (N_{A}) indicates the site of bonding of to NR₄ₐ of formula (I) and (N_{B}) indicates the site of bonding to NR_{4c} of formula (I);
R₁ is selected from the group consisting of —(CH₂)ₛCH₃, —CH(CH₃)(CH₂)ₜCH₃, —(CH₂)ᵤCH(CH₃)₂, —C(CH₃)₃, —CH₂—C(CH₃)₃, —CHR₁₇OR₁₈, 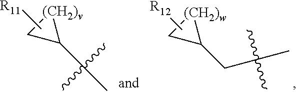
wherein s is 0, 1, 2, 3 or 4; t is 1, 2 or 3; u is 0, 1 or 2; v is 1, 2, 3 or 4; w is 1, 2, 3 or 4; and R₁₁ and R₁₂ are optionally present and, when present, are independently selected from the group consisting of C₁-C₄ alkyl, hydroxyl and alkoxy; R₁₇ is hydrogen or methyl; and R₁₈ is selected from the group consisting of hydrogen, C₁-C₄ alkyl and acyl;
R₂ₐ is selected from the group consisting of —CH₃, —CH₂CH₃, —CH(CH₃)₂, —CF₃, —CF₂H and —CH₂F;
R_{2b} is selected from the group consisting of —H and —CH₃;
R₃ₐ is selected from the group consisting of hydrogen, C₁-C₄ alkyl, hydroxyl and alkoxy;
R_{3b} is selected from the group consisting of hydrogen and C₁-C₄ alkyl;
R₄ₐ, R_{4b}, R_{4c} and R_{4d} are independently selected from the group consisting of hydrogen and C₁-C₄ alkyl;
R₅, when Y₁ is O or NR₁₆, is selected from the group consisting of hydrogen, C₁-C₄ alkyl and acyl; or, when Y₁ is C(═O), is selected from the group consisting of hydroxyl, alkoxy and amine;
R₆ is selected from the group consisting of hydrogen, C₁-C₄ alkyl, oxo and trifluoromethyl;
R₇ is selected from the group consisting of hydrogen, C₁-C₄ alkyl, hydroxyl, alkoxy and trifluoromethyl; or R₇ and X₁ together with the carbons to which they are bonded form a five or six-membered ring;
R₁₀ is selected from the group consisting of hydrogen, C₁-C₄ alkyl, 1,1,1-trifluoroethyl, hydroxyl and alkoxy, with the provisos that when L₆ is CH, R₁₀ is also selected from trifluoromethyl and when L₆ is N, R₁₀ is also selected from sulfonyl; or R₁₀ and R₈ₐ together form a five- or six-membered ring;
R₂₆, R₂₈ and R₂₉ are independently selected from the group consisting of hydrogen, C₁-C₄ alkyl, hydroxyl, alkoxy and trifluoromethyl; or R₂₈ and R₂₉ together form a three-membered ring;
R₂₇ is selected from the group consisting of hydrogen, C₁-C₄ alkyl, hydroxyl, alkoxy and trifluoromethyl; or R₂₇ and X₄₃ together with the carbons to which they are bonded form a five or six-membered ring
R₃₀ is selected from the group consisting of hydrogen, C₁-C₄ alkyl, hydroxyl, alkoxy and trifluoromethyl;
Ar is selected from the group consisting of: 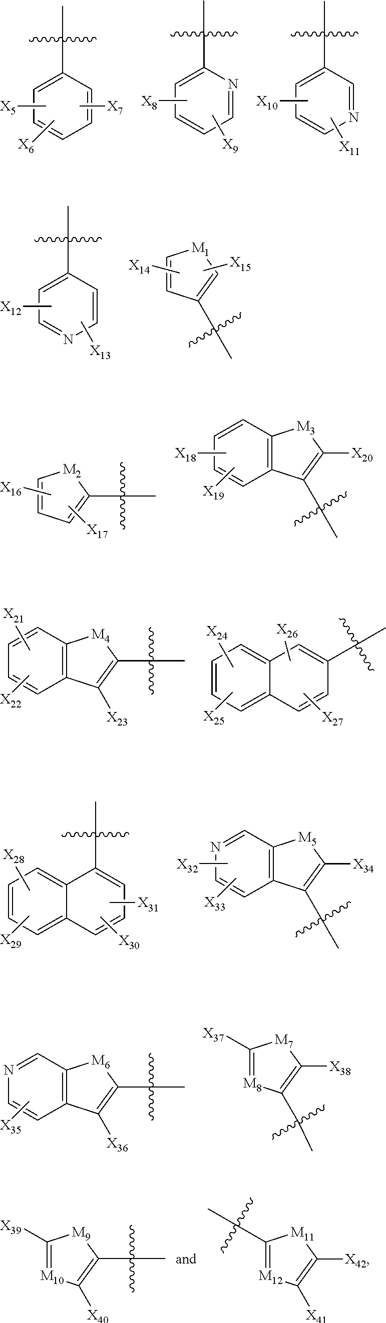
wherein M₁, M₂, M₃, M₄, M₅, M₆, M₇, M₉ and M₁₁ are independently selected from the group consisting of O, S and NR₁₃, wherein R₁₃ is selected from the group consisting of hydrogen, C₁-C₄ alkyl, formyl, acyl and sulfonyl; M₈, M₁₀ and M₁₂ are independently selected from the group consisting of N and CR₁₄, wherein R₁₄ is selected from the group consisting of hydrogen and C₁-C₄ alkyl; X₅, X₆, X₇, X₁₈, X₁₉, X₂₁, X₂₂, X₂₄, X₂₅, X₂₆, X₂₇, X₂₈, X₂₉, X₃₀ and X₃₁ are independently selected from the group consisting of hydrogen, halogen, trifluoromethyl and C₁-C₄ alkyl; and X₈, X₉, X₁₀, X₁₁, X₁₂, X₁₃, X₁₄, X₁₅, X₁₆, X₁₇, X₂₀, X₂₃, X₃₂, X₃₃, X₃₄, X₃₅, X₃₆, X₃₇, X₃₈, X₃₉, X₄₀, X₄₁ and X₄₂ are independently selected from the group consisting of hydrogen, hydroxyl, alkoxy, amino, halogen, cyano, trifluoromethyl and C₁-C₄ alkyl;
L₁, L₂, L₃, L₄ and L₆ are independently selected from the group consisting of CH and N;
L₅ is selected from the group consisting of CR₁₅ₐR_{15b}, O and NR_{15c}, wherein R₁₅ₐ and R_{15b} are independently selected from hydrogen, C₁-C₄ alkyl, hydroxyl and alkoxy; and R_{15c} is selected from the group consisting of hydrogen, C₁-C₄ alkyl, acyl and sulfonyl;
L₁₀ is selected from the group consisting of CR₃₅ₐR_{35b}, O and OC(═O)O, wherein R₃₅ₐ and R_{35b} are independently selected from hydrogen, C₁-C₄ alkyl, hydroxyl and alkoxy;
X₁ is selected from the group consisting of hydrogen, halogen, trifluoromethyl and C₁-C₄ alkyl; or X₁ and R₇ together form a five or six-membered ring;
X₂, X₃ and X₄ are independently selected from the group consisting of hydrogen, halogen, trifluoromethyl and C₁-C₄ alkyl;
X₄₃ and X₄₄ are optionally present and, when present, are independently selected from the group consisting of C₁-C₄ alkyl, hydroxyl, alkoxy and trifluoromethyl; or X₄₃ and R₂₇ together form a five or six-membered ring; and
Y₁ is selected from the group consisting of C(═O), O and NR₁₆, wherein R₁₆ is selected from the group consisting, of hydrogen, C₁-C₄ alkyl, acyl and sulfonyl;
z is 0, 1, 2 or 3; and
Z is selected from the group consisting of (Ar)-CHR₈ₐCHR₉ₐ-(L₆), (Ar)-CR_{8b}═CR_{9b}-(L₆) and -(Ar)-C≡C-(L₆), wherein (Ar) indicates the site of bonding to the phenyl ring and (L₆) the site of bonding to L₆, R₈ₐ and R₉ₐ are independently selected from the group consisting of hydrogen, C₁-C₄ alkyl, hydroxyl, alkoxy, oxo and trifluoromethyl; R_{8b} and R_{9b} are independently selected from the group consisting of hydrogen, C₁-C₄ alkyl, fluoro, hydroxyl, alkoxy and trifluoromethyl; or R₈ₐ and R₉ₐ together form a three-membered ring; or R₈ₐ and R₁₀ together form a five- or six-membered ring; or R₈ₐ and X₄ together form a five- or six-membered ring; or R₉ₐ and X₄ together form a five- or six-membered ring; or R_{8b} and X₄ together form a five- or six-membered ring; or R_{9b} and X₄ together form a five- or six-membered ring.

2. The compound of formula (I) of claim 1, wherein
R₁ is —CH(CH₃)CH₂CH₃, —CH(CH₃)₂, 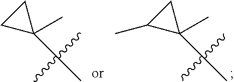
R₂ₐ and R_{2b} are each —CH₃;
R₃ₐ is hydrogen or —CH₃;
R_{2b}, R_{3b}, R_{4b}, R_{4c}, R_{4d}, R₅, R₆ and R₇ are each hydrogen;
R₉ is hydrogen or hydroxyl;
R₁₀ is —CH₃ or —CH₂CH₃;
Ar is 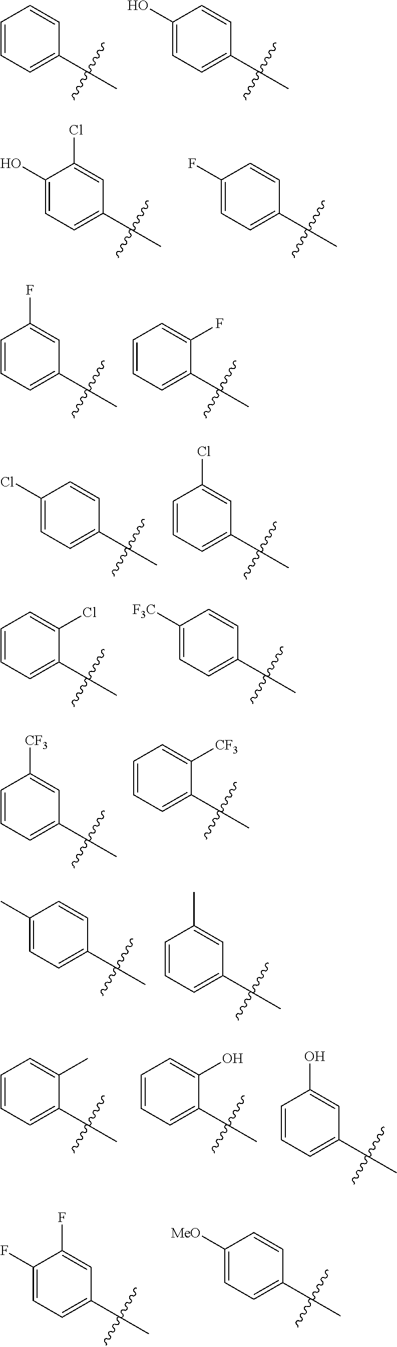 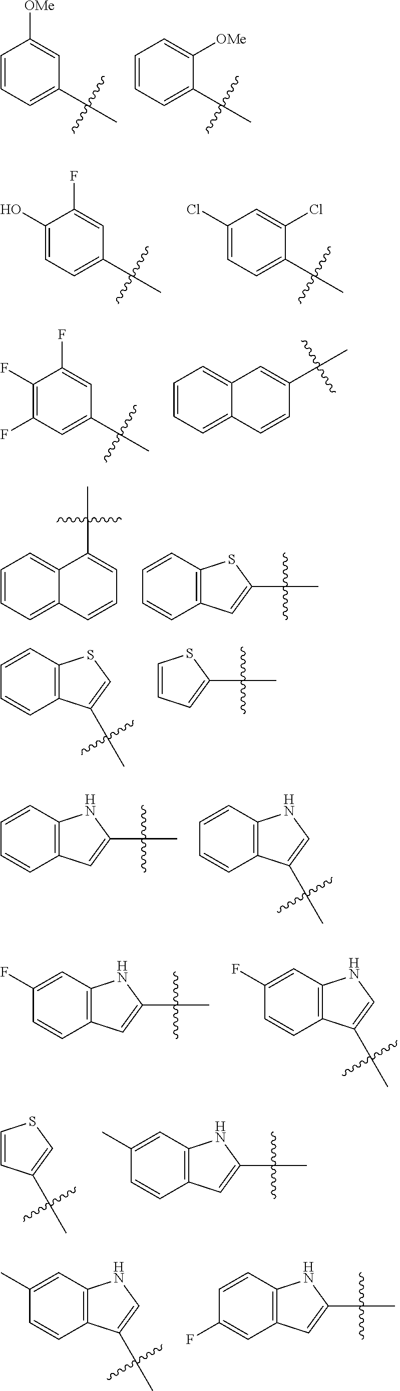 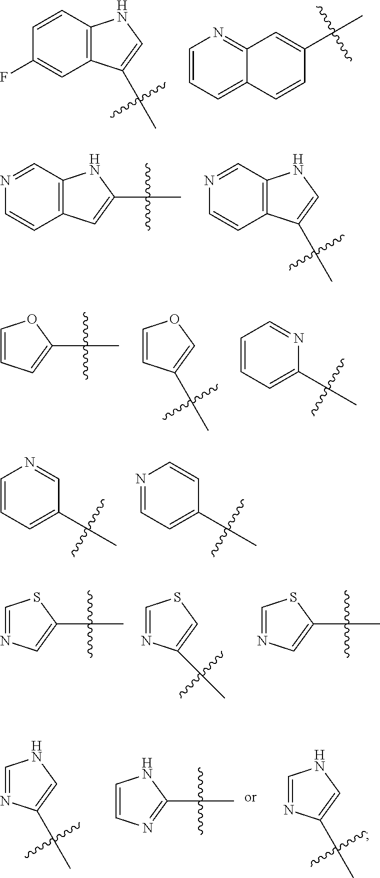
L₁, L₂, L₃, L₄, L₅ and L₆ are each CH;
X₁ is fluoro and X₂, X₃ and X₄ are hydrogen; or X₂ is fluoro and X₁, X₃ and X₄ are hydrogen; or X₃ is fluoro and X₁, X₂ and X₄ are hydrogen; or X₄ is fluoro and X₁, X₂ and X₃ are hydrogen, or X₂ and X₃ are fluoro and X₁ and X₄ are hydrogen;
Y is O; and
Z is CH₂CH₂ or C≡C;
or a pharmaceutically acceptable salt thereof.

3. The compound of formula (I) of claim 1, wherein T is selected from the group consisting of: 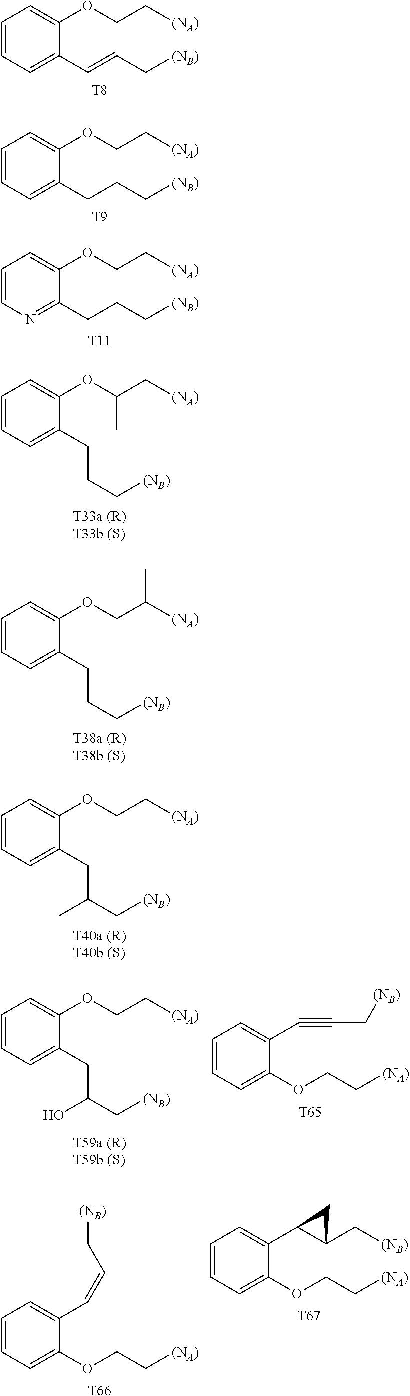 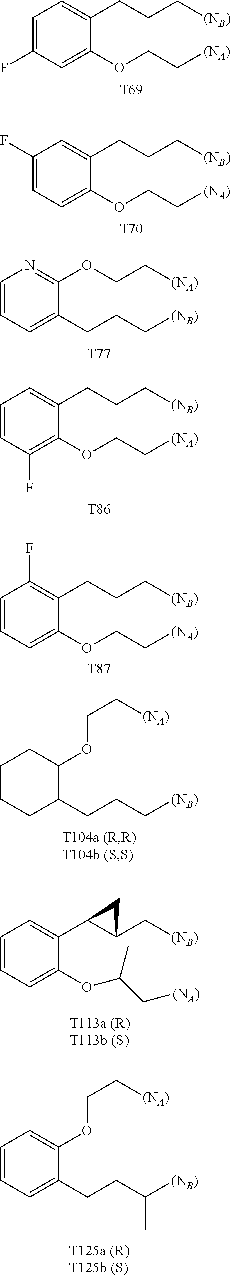 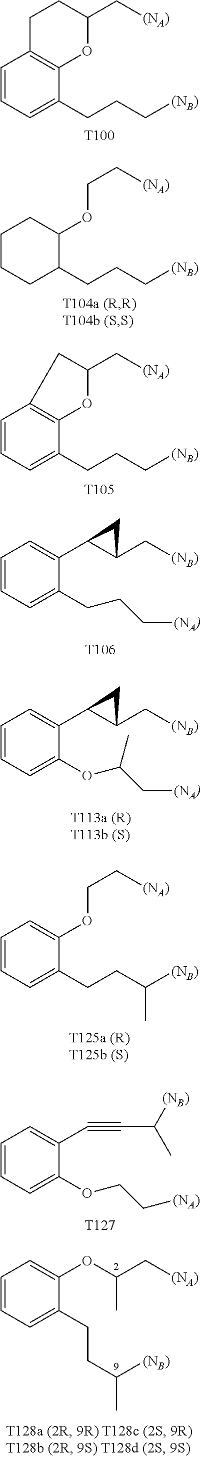 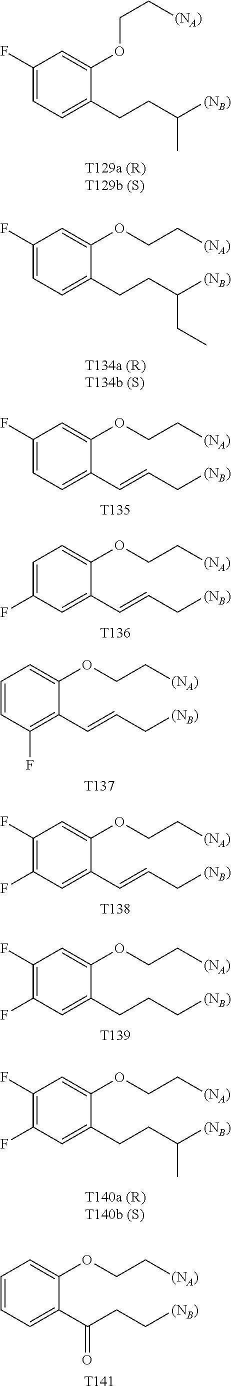 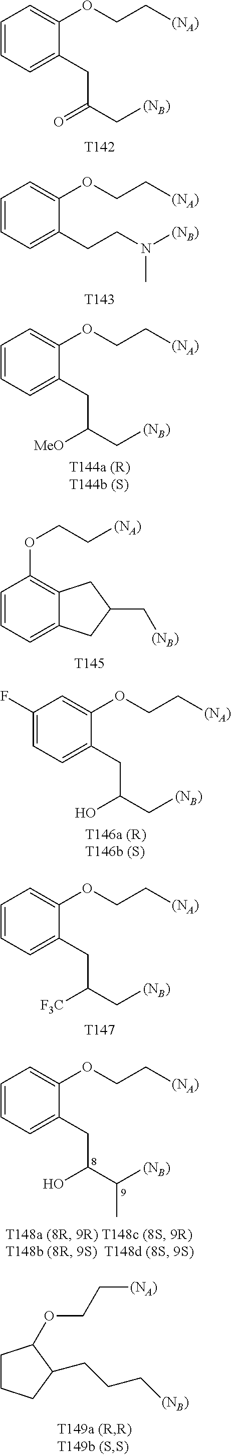 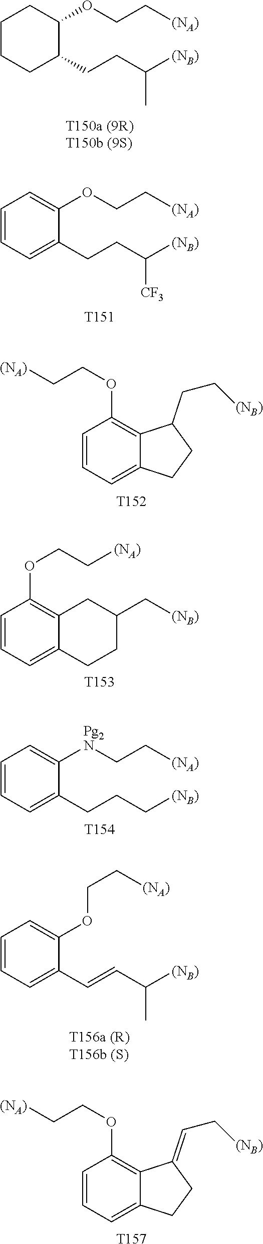 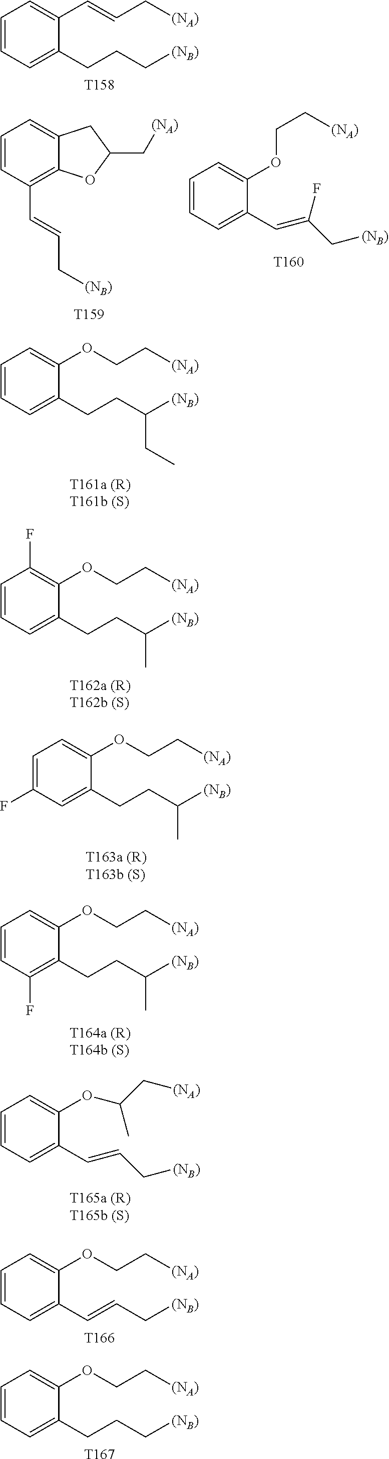 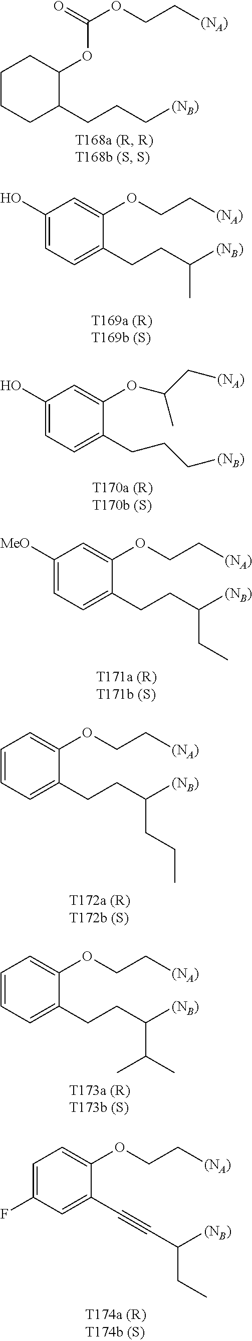 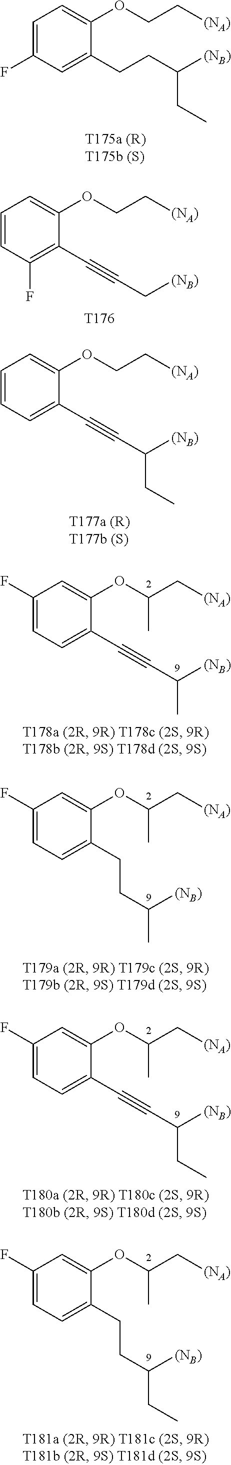 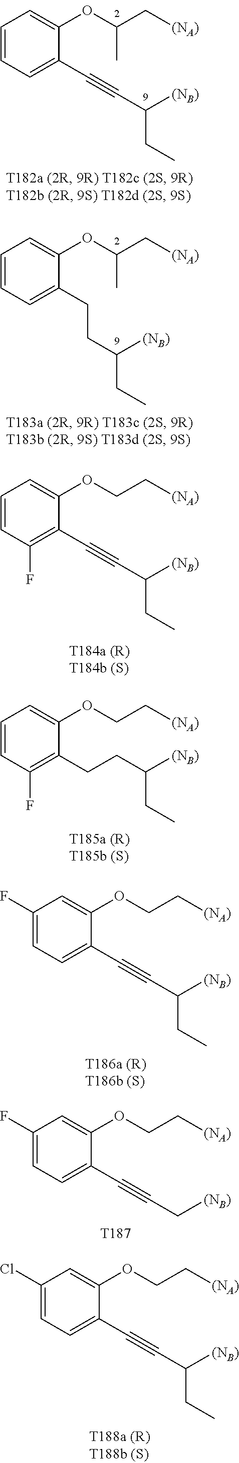 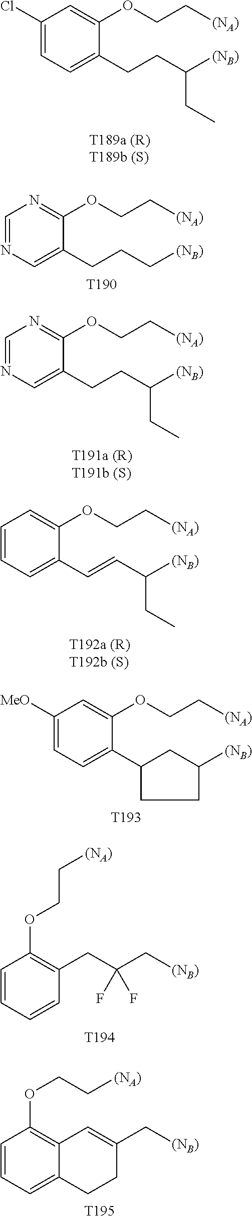 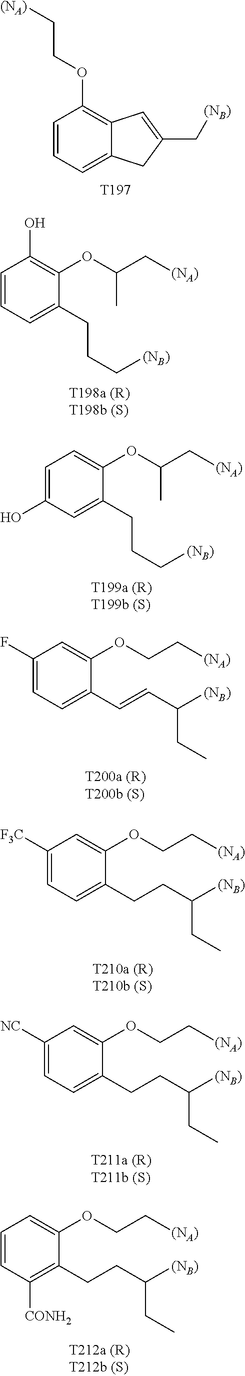 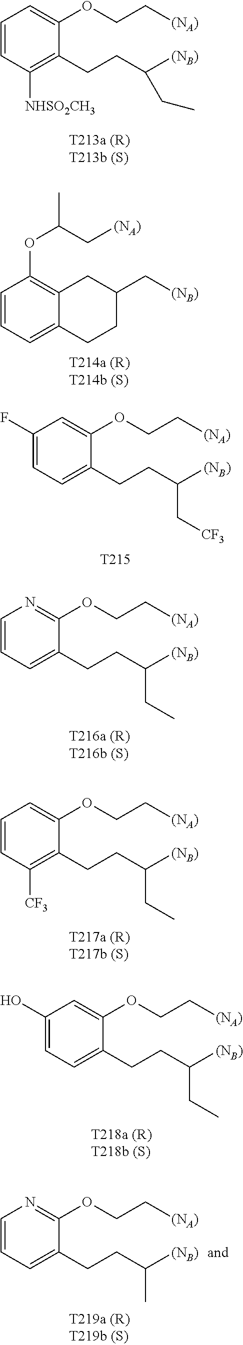 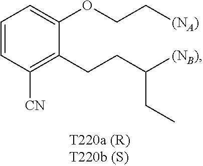
wherein (N_{A}) indicates the site of bonding of to NR₄ₐ of formula (I), (N_{B}) indicates the site of bonding to NR_{4c} of formula (I) and Pg is a nitrogen protecting group.

4. The compound of claim 1 with the following structure: 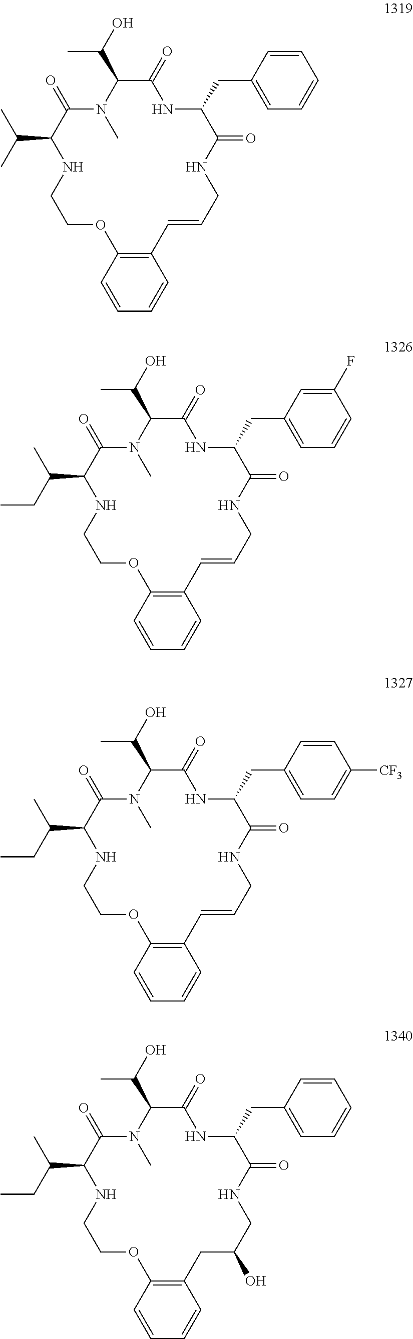 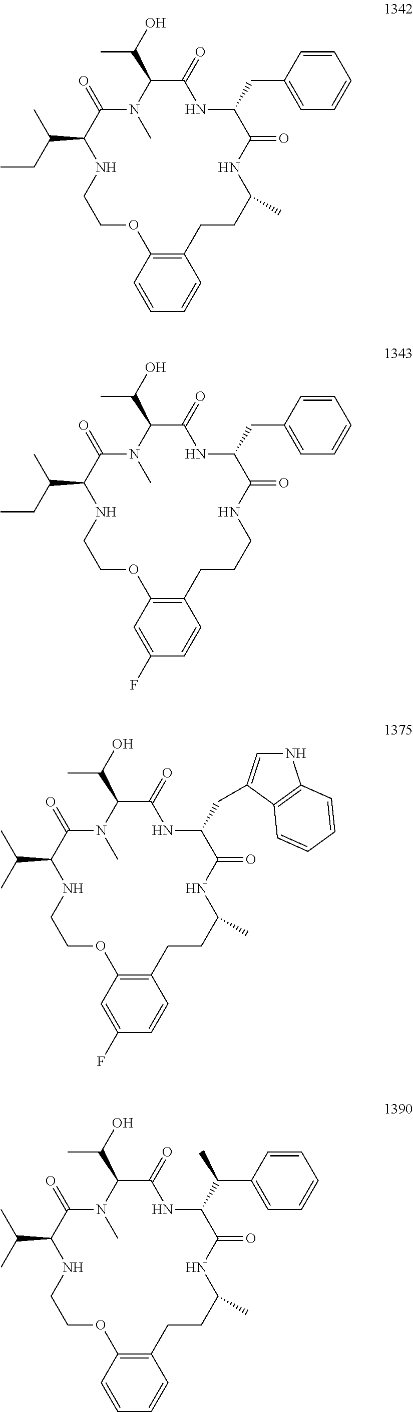 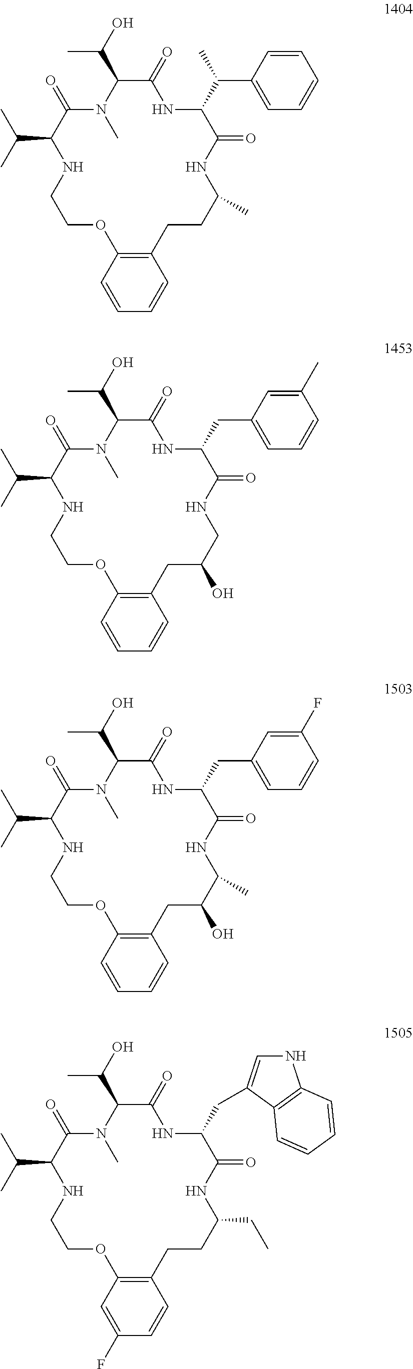 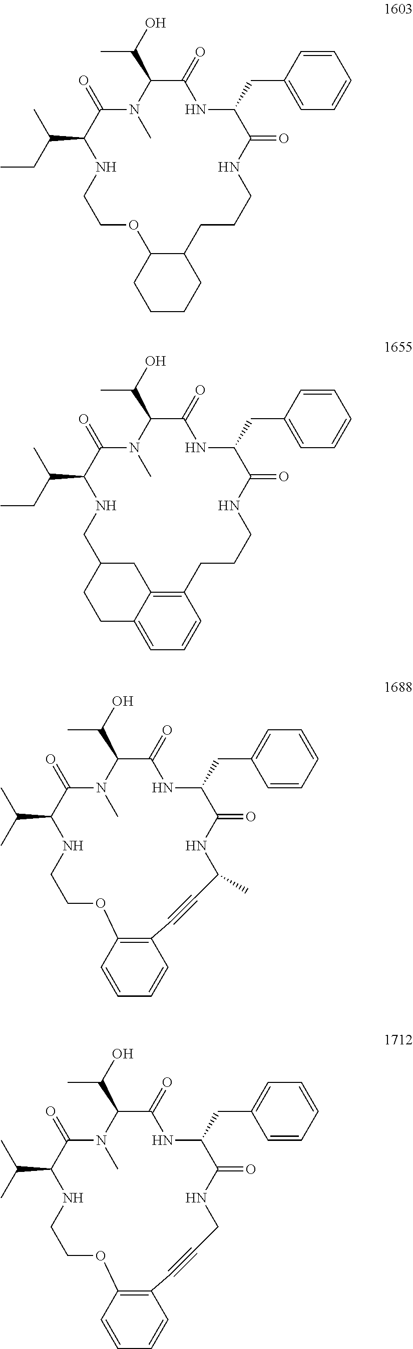 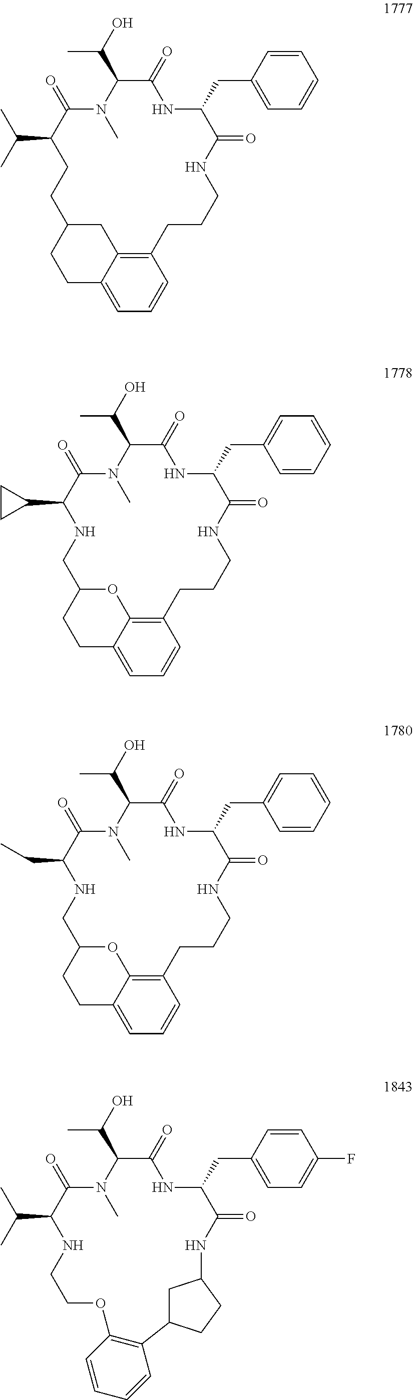 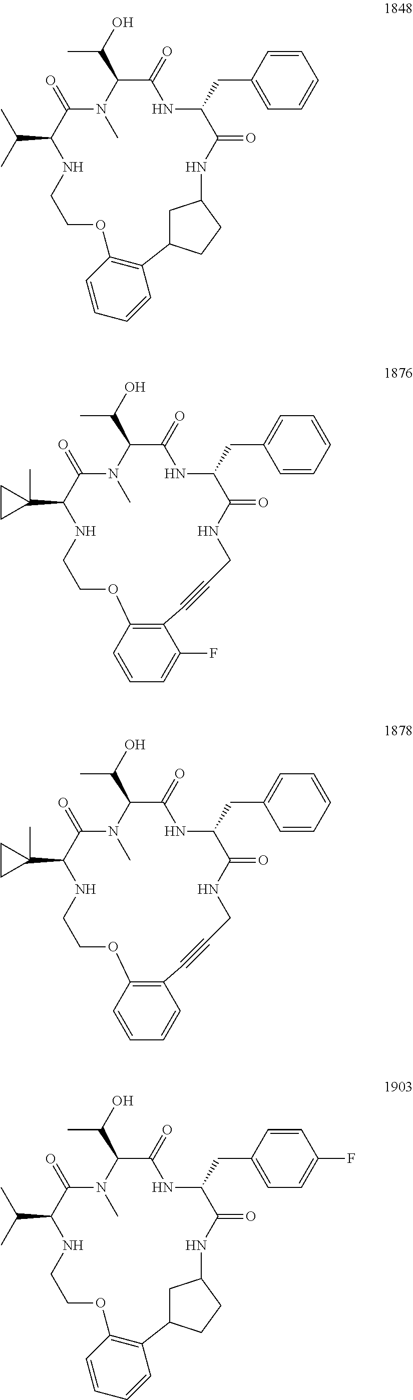 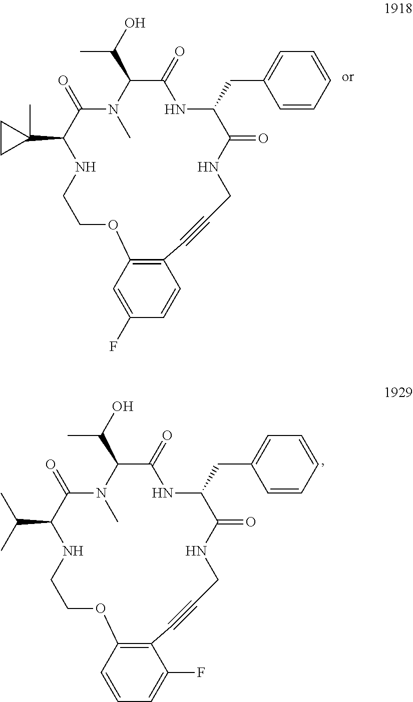
or a pharmaceutically acceptable salt thereof.

5. A pharmaceutical composition comprising:
(a) a compound of claim 1; and
(b) a pharmaceutically acceptable carrier, excipient or diluent.

6. A pharmaceutical composition comprising:
(a) a compound of claim 4; and
(b) a pharmaceutically acceptable carrier, excipient or diluent.

7. A pharmaceutical composition comprising:
(a) a compound of claim 1;
(b) one or more additional therapeutic agents and
(c) a pharmaceutically acceptable carrier, excipient or diluent.

8. The pharmaceutical composition of claim 7, wherein the additional therapeutic agent is selected from the group consisting of a GLP-1 agonist, a DPP-IV inhibitor, an amylin agonist, a PPAR-α agonist, a PPAR-γ agonist, a PPAR-α/γ dual agonist, a GDIR or GPR119 agonist, a PTP-1B inhibitor, a peptide YY agonist, an 11β-hydroxysteroid dehydrogenase (11β-HSD)-1 inhibitor, a sodium-dependent renal glucose transporter type 2 (SGLT-2) inhibitor, a glucagon antagonist, a glucokinase activator, an α-glucosidase inhibitor, a glucocorticoid antagonist, a glycogen synthase kinase 3β (GSK-3β) inhibitor, a glycogen phosphorylase inhibitor, an AMP-activated protein kinase (AMPK) activator, a fructose-1,6-biphosphatase inhibitor, a sulfonyl urea receptor antagonist, a retinoid X receptor activator, a 5-HT₁ₐ agonist, a 5-HT_{2c} agonist, a 5-HT₆ antagonist, a cannabioid antagonist or inverse agonist, a melanin concentrating hormone-1 (MCH-1) antagonist, a melanocortin-4 (MC4) agonist, a leptin agonist, a retinoic acid receptor agonist, a stearoyl-CoA desaturase-1 (SCD-1) inhibitor, a neuropeptide Y Y2 receptor agonist, a neuropeptide Y Y4 receptor agonist, a neuropeptide Y Y5 receptor antagonist, a neuronal nicotinic receptor α₄β₂ agonist a diacylglycerol acyltransferase 1 (DGAT-1) inhibitor, a thyroid receptor agonist, a lipase inhibitor, a fatty acid synthase inhibitor, a glycerol-3-phosphate acyltransferase inhibitor, a CPT-1 stimulant, an α_{1A}-adrenergic receptor agonist, an α_{2A}-adrenergic receptor agonist, a β₃-adrenergic receptor agonist, a histamine H3 receptor antagonist, a cholecystokinin A receptor agonist and a GABA-A agonist.

9. The pharmaceutical composition of claim 8 wherein the GLP-1 agonist is selected from the group consisting of GLP-1, GLP-1 (7-36) amide, exenatide (exendin-4), liraglutide (NN2211), gilatide, albiglutide (GSK-716155, albugon), taspoglutide, GLP1-I.N.T., GLP-1 DUROS, AC2592, AC2993 LAR, ADX4 (PAM), ARI-2255, ARI-2651, BRX-0585 (GLP-1-Tf), CJC-1131, CJC-1134-PC(PC-DAC™:Exendin-4), CS-872, AVE-0010 (ZP-10), BIM-51077 (R-1583), BIM-51182, DA3071, GTP-010, ITM-077, SUN E7001, TH-0318, TH-0396, TTP-854, LY-315902 and LY-307161.

10. The pharmaceutical composition of claim 8 wherein the DPP-IV inhibitor is selected from the group consisting of sitagliptin, vidagliptin, saxagliptin (BMS-477118), alogliptin (SYR322), ABT-279, ALS-20426, AR12243, AM622, ASP8497, DA 1229, DB295, E3024, FE999011, GRC-8200, KR-62436, KRP104, MP-513, PHX1149, PSN9301, SK-0403, SYR619, TA-6666, TAK 100 and VMD-700.

11. The pharmaceutical composition of claim 8 wherein the amylin agonist is selected from the group consisting of amylin, pramlintide, MBP-0250 and PX811016.

12. The pharmaceutical composition of claim 8 wherein the PPAR-γ agonist is selected from the group consisting of pioglitazone, rivoglitazone, rosiglitazone and troglitazone.

13. The pharmaceutical composition of claim 8 wherein the agonist is a PPAR-α/γ dual agonist selected from the group consisting of ragaglitazar, tesaglitazar, muraglitazar, aleglitazar, cevoglitazar, R1439, PLX204 (PPM-204).

14. The pharmaceutical composition of claim 8 wherein the PTP-1B inhibitor is selected from the group consisting of ISIS 113715 and KR61639.

15. The pharmaceutical composition of claim 8 wherein the 5-HT2c agonist is selected from the group consisting of lorcaserin, vabicaserin (SCA-136), ATHX-105, BVT933 (GW 876167), IK264, LY448100, MK-212, ORG-12962, VR1065, WAY-163909 and YM348.

16. The pharmaceutical composition of claim 8 wherein the cannabioid antagonist or inverse agonist is selected from the group consisting of rimonabant, taranabant (MK-0364), surinabant, AVE1625, AVN 342, CP-945,598, E-6776, GRC 10389, SLV-319, SR 147778, TM38837 and V24343.

17. The pharmaceutical composition of claim 8 wherein the peptide YY agonist is selected from the group consisting of peptide YY and peptide YY 3-36 (AC-162352).

18. The pharmaceutical composition of claim 8 wherein the lipase inhibitor is selected from the group consisting of orlistat and cetilistat.

19. The pharmaceutical composition of claim 8 wherein the α-glucosidase inhibitor is selected from the group consisting of acarbose, miglitol and voglibose.

20. The pharmaceutical composition of claim 8 wherein the SGLT-2 inhibitor is selected from the group consisting of dapagliflozin, remogliflozin, sergliflozin, AVE2268, GSK189075.

21. The pharmaceutical composition of claim 8 wherein the 11β-HSD-1 inhibitor is selected from the group consisting of INCB13739, BVT.3498, BVT.2733, AMG 221, PF-915275.

22. The pharmaceutical composition of claim 8 wherein the glucokinase inhibitor is selected from the group consisting of R1440/GK3, RO-28-1675, PSN010 and ARRY-403.

23. The pharmaceutical composition of claim 8 wherein the additional therapeutic agent is selected from the group consisting of metformin, sibutramine, phentermine, betahistine, methamphetamine, benzphetamine, phendimetrazine, diethylpropion, bupropion, topiramate, carbutamide, chlorpropamide, glibenclamide (glyburide), gliclazide, glimepiride, glipizide, gliquidone, mitiglinide, nateglinide, repaglinide, tolazamide, tolbutamide; and pharmaceutically acceptable salts thereof.

24. A kit comprising one or more containers comprising pharmaceutical dosage units further comprising an effective amount of one or more compounds of claim 1 or a pharmaceutically acceptable salt thereof, wherein the container is packaged with optional instructions for the use thereof.

25. A method of modulating GRLN (GHS-R1a) receptor activity in a mammal comprising administering to said mammal an effective GRLN (GHS-R1a) receptor activity modulating amount of a compound of claim 1.

26. A method of treating a metabolic and/or endocrine disorder comprising administering to a subject in need thereof an effective amount of a compound of claim 1.

27. The method of claim 26, wherein the metabolic and/or endocrine disorder is selected from the group consisting of obesity or an obesity-associated condition, diabetes, metabolic syndrome, non-alcoholic fatty acid liver disease (NAFLD), non-alcoholic steatohepatitis (NASH) and steatosis.

28. A method of treating an appetite or eating disorder comprising administering to a subject in need thereof an effective amount of a compound of claim 1.

29. The method of claim 28, wherein the appetite or eating disorder is Prader-Willi syndrome or hyperphagia.

30. The method of claim 29, wherein the hyperphagia is diabetic hyperphagia.

31. A method of treating an addictive disorder comprising administering to a subject in need thereof an effective amount of a compound of claim 1.

32. The method of claim 31, wherein the addictive disorder comprises alcohol dependence, drug dependence and/or chemical dependence.

33. A method of treating a cardiovascular disease comprising administering to a subject in need thereof an effective amount of a compound of claim 1.

34. A method of treating a gastrointestinal disorder comprising administering to a subject in need thereof an effective amount of a compound of claim 1.

35. A method of treating a genetic disorder comprising administering to a subject in need thereof an effective amount of a compound of claim 1.

36. A method of treating a hyperproliferative disorder comprising administering to a subject in need thereof an effective amount of a compound of claim 1.

37. A method of treating an inflammatory disorder comprising administering to a subject in need thereof an effective amount of a compound of claim 1.

38. A method of treating a central nervous system (CNS) disorder comprising administering to a subject in need thereof an effective amount of a compound of claim 1.

39. A macrocyclic compound selected from the group consisting of 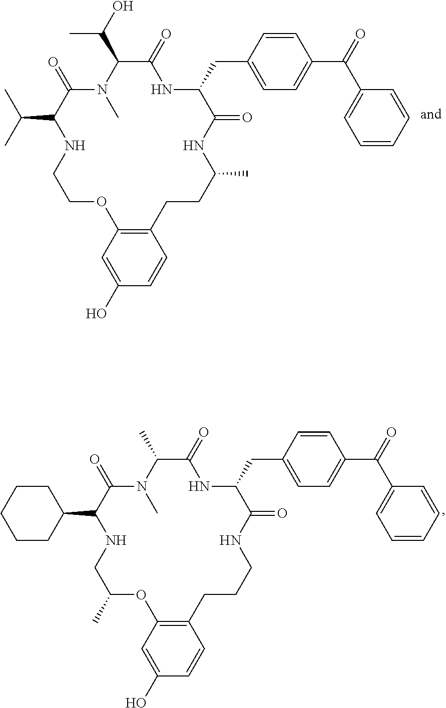
or a pharmaceutically acceptable salt thereof.
